SEQUENCE LISTING (278 sequences)

SEQ ID NO: 1 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - DNA, 2211 bp, Adeno-associated virus 9
  source 1..2211: mol_type = genomic DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 3 - protein, 735 aa, Adeno-associated virus 2 [sequence omitted]

SEQ ID NO: 4 - DNA, 2208 bp, Adeno-associated virus 2
  source 1..2208: mol_type = genomic DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 5 - protein, 724 aa, Adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 6 - DNA, 2175 bp, Adeno-associated virus 5
  source 1..2175: mol_type = genomic DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacgggctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 7 - protein, 738 aa, Adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 8 - DNA, 2217 bp, Adeno-associated virus 8
  source 1..2217: mol_type = genomic DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 9 - protein, 738 aa, Adeno-associated virus
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 10 - DNA, 2217 bp, Adeno-associated virus
  source 1..2217: mol_type = genomic DNA
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagaggccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgtgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 11 - protein, 738 aa, Adeno-associated virus
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFTKAKLASFITQYSTGQVSVEIEWELQKENSKRWNPEIQYTSNYYKSTNVDFAVNTEGTYSEPRPIGTRYLTRNL

SEQ ID NO: 12 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQTQAQAGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 13 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRENVDADKVMITNEEEIKTTNPVATESYGTVATNHQSAQAQASVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSASAQAQAGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTYRDNVDADKVMITNEEEIKTTNPVATESYGTVATNHQSAQAQAQVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVSITNEEEIKTTNPVATESYGTVATNIQSAQAQAIVGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 17 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVNITNEEEIKTTNPVATESYGVVATNHQSAQAQAQSGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 18 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAGAQAQSGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 19 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESHGVVCTNHQSAQAQAQVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 20 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMLTNEEEIKTTNPVATESYGVVATNHQSAQTQAQAGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 21 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVAITNEEEIKTTNPVATESYGVVATNMQSAQAQASVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 22 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSANAQAQAGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 23 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVVITNEEEIKTTNPVATESYGVVATNHQSAQAQAQSGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 24 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMQTNEEEIKTTNPVATESYGTVATNHQSAQAQAQVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 25 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNHQSANTQASVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 26 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVQITNEEEIKTTNPVATESYGVVATNQQSAQAQAQVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 27 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAIRGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 28 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVCITNEEEIKTTNPVATESYGVVATNHQSAQAQAQAGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 29 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTTRDNVDADKVMITNEEEIKTTNPVATESYGTVATNHQSAQAQAQVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 30 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVAINNQSAQAQAQVGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 31 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTARDNVDADKVMITNEEEIKTTNPVATESYGVVATNHQSAQAQAQSGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 32 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGNDNVDADKVMITNEEEIKTTNPVATESTGVVATNHQSAQAQASVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 33 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGKDNVDADKVMITNEEEIKTTNPVATESYGVVATNHQSAQSQAQAGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 34 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMISNEEEIKTTNPVATESYGVVATNIQSAQAQASVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 35 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNIQSAQAQASVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 36 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMISNEEEIKTTNPVATESYGVVATNHQSAQAQAQAGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 37 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVAITNEEEIKTTNPVATESYGVVATNHQSAQAQASVGALQTQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 38 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESTGVVATNHQSAQAQASVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 39 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNSDADKVMITNEEEIKTTNPVATESYGTVATNHQSAQAQAQVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 40 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNHQSHNTQAIVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 41 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADNVMITNEEEIKTTNPVATESYGVVATNLQSAQTQASVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 42 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNLQSAQAQAQVGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 43 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSATAQAQAGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 44 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESHGVVAMNHQSAQAQASVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 45 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTFRDNVDADKVMITNEEEIKTTNPVATESYGTVATNHQSAQAQAQVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 46 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEWYGVVATNHQSSQTQAQVGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 47 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGKDNVDADKVMITNEEEIKTTNPVATESYGVVATNHQSAQSQAQSGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 48 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTIRDNVDADKVMITNEEEIKTTNPVATESYGVVATNHQSAQAQAQSGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 49 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTHRDNVDADKVMITNEEEIKTTNPVATESYGTVATNHQSAQAQAQVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 50 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVQITNEEEIKTTNPVATESYGVVAINHQSAQAQAQVGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 51 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQVGALQTQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 52 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESTGVVATNHQSAQSQAAVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 53 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNNQSAQAQAQVGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 54 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSANAQAQVGALQTQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 55 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSANAQAQAGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 56 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNHQSANTQANVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 57 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGLDNVDADKVMITNEEEIKTTNPVATESYGTVATNHQSAQAQAQVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 58 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADNVMITNEEEIKTTNPVATESYGVVATNHQSANAQAQAGALQSQGVLPGMVWQNRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 59 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNHQQSQAQAQVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 60 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNHQSANTQAAVGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 61 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSSQAQAQSGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 62 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGTVATNHQSANTQAQVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 63 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTNRDNVDADKVMITNEEEIKTTNPVATESYGTVATNHQSSQTQAQVGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 64 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADLVQITNEEEIKTTNPVATESYGVVATNHQSAQAQAQAGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 65 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATEWYGTVNTNHQSAQAQAQVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 66 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNNQSAQAQAQVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 67 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVAITNEEEIKTTNPVATESYGTVATNHQQTQAQAQVGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 68 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVTITNEEEIKTTNPVATESYGVVATNHQSSNAQAQSGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 69 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVIITNEEEIKTTNPVATESYGSVATNHQSAQAQAATGWLQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 70 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGSVATNLQSAQAQAQVGYLQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 71 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGSVATNHQSAQAQPQTGWLQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 72 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATWSYGQVATNNQSAQAQAQVGALQSQGVLPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 73 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATSSYGQVATNMQSAQAQAQVGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 74 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggagttgtggccacaaaccaccagagtgcccaaactcaggcgcaggccggcgctttgcaaagccaaggagtacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 75 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagaaaacgtggatgcggacaaagtcatgataaccaacgaagaagaaattaaaactactaacccggtagcaacggagtcctatggaactgtggccacaaaccaccagagtgcccaagcacaggcgtctgttggcgctcttcaatcccaaggagtccttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 76 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggagtcgtggccacaaaccaccagagtgcctcggcacaggcgcaggcaggcgccttacaatcgcaaggagttcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 77 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaacttatagagacaacgtggatgcggacaaagtcatgataaccaacgaagaagaaattaaaactactaacccggtagcaacggagtcctatggaacggtggccacaaaccaccagagtgcccaagcacaggcgcaggtcggcgccctgcaaagccaaggagttcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 78 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcagtataaccaacgaagaagaaattaaaactactaacccggtagcaacggagtcctatggaaccgtggccacaaacattcagagtgcccaagcacaggcgatagtaggcgctctacaaagccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 79 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcaatataaccaacgaagaagaaattaaaactactaacccggtagcaacggagtcctatggagtggtggccacaaaccaccagagtgcccaagcacaggcgcagtcgggcgccttgcaatcccaaggagttcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 80 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggagttgtggccacaaaccaccagagtgccggtgcacaggcgcagagcggcgctctccaaagtcaaggagtccttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 81 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcccatggagttgtgtgcacaaaccaccagagtgcccaagcacaggcgcaggtgggcgccttgcaatctcaaggagtacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 82 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgctgaccaacgaagaagaaattaaaactactaacccggtagcaacggagtcctatggagttgtggccacaaaccaccagagtgcccaaacccaggcgcaggcgggcgccttacaatcccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 83 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcgctataaccaacgaagaagaaattaaaactactaacccggtagcaacggagtcctatggagtcgtggccacaaacatgcagagtgcccaagcacaggcgtccgttggcgcgctgcaaagccaaggagtgcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 84 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggagttgtggccacaaaccaccagagtgccaacgcacaggcgcaggcgggcgcattgcaatcacaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 85 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcgtaataaccaacgaagaagaaattaaaactactaacccggtagcaacggagtcctatggagtggtggccacaaaccaccagagtgcccaagcacaggcgcagagtggcgctctgcaatcacaaggagtccttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 86 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgcagaccaacgaagaagaaattaaaactactaacccggtagcaacggagtcctatggaactgtggccacaaaccaccagagtgcccaagcacaggcgcaggtgggcgccctacaaagtcaaggagttcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 87 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggaaccgtggccacaaaccaccagagtgccaatacgcaggcgtccgtaggcgcactgcaaagtcaaggagtacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 88 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtccagataaccaacgaagaagaaattaaaactactaacccggtagcaacggagtcctatggagtcgtggccacaaaccagcagagtgcccaagcacaggcgcaggtcggcgctcttcaaagccaaggagtacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 89 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggagtcgtggccacaaaccaccagagtgcccaagcacaggcgattaggggcgccttacaatcgcaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 90 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtctgcataaccaacgaagaagaaattaaaactactaacccggtagcaacggagtcctatggagtggtggccacaaaccaccagagtgcccaagcacaggcgcaggcgggcgctctacaaagtcaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 91 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactacaagagacaacgtggatgcggacaaagtcatgataaccaacgaagaagaaattaaaactactaacccggtagcaacggagtcctatggaacggtggccacaaaccaccagagtgcccaagcacaggcgcaggttggcgcccttcaatcacaaggagttcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 92 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggagtcgtggccattaacaatcagagtgcccaagcacaggcgcaggtcggcgcgctccaaagtcaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 93 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactgcgagagacaacgtggatgcggacaaagtcatgataaccaacgaagaagaaattaaaactactaacccggtagcaacggagtcctatggagttgtggccacaaaccaccagagtgcccaagcacaggcgcagagtggcgctctacaatcgcaaggagtccttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 94 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaaatgacaacgtggatgcggacaaagtcatgataaccaacgaagaagaaattaaaactactaacccggtagcaacggagtccaccggagttgtggccacaaaccaccagagtgcccaagcacaggcgtcagttggcgcattacaaagtcaaggagttcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 95 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaaaagacaacgtggatgcggacaaagtcatgataaccaacgaagaagaaattaaaactactaacccggtagcaacggagtcctatggagttgtggccacaaaccaccagagtgcccaaagccaggcgcaggctggcgcgctccaatctcaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 96 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataagtaacgaagaagaaattaaaactactaacccggtagcaacggagtcctatggagtcgtggccacaaacatccagagtgcccaagcacaggcgagtgtgggcgcgttgcaatcacaaggagtacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 97 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggagttgtggccacaaacattcagagtgcccaagcacaggcgtccgtcggcgcacttcaatctcaaggagttcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 98 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgatttccaacgaagaagaaattaaaactactaacccggtagcaacggagtcctatggagtagtggccacaaaccaccagagtgcccaagcacaggcgcaggcgggcgcgttgcaaagtcaaggagtgcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 99 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcgcgataaccaacgaagaagaaattaaaactactaacccggtagcaacggagtcctatggagtcgtggccacaaaccaccagagtgcccaagcacaggcgtcagtgggcgcactacaaacacaaggagtgcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 100 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtccactggagtagtggccacaaaccaccagagtgcccaagcacaggcgtccgtaggcgctttacaatcacaaggagttcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 101 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacagcgatgcggacaaagtcatgataaccaacgaagaagaaattaaaactactaacccggtagcaacggagtcctatggaacggtggccacaaaccaccagagtgcccaagcacaggcgcaggtcggcgcactgcaatctcaaggagtccttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 102 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggaactgtggccacaaaccaccagagtcacaacactcaggcgatcgtaggcgcactgcaaagccaaggagtgcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 103 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaatgtcatgataaccaacgaagaagaaattaaaactactaacccggtagcaacggagtcctatggagtggtggccacaaacttacagagtgcccaaacccaggcgtcagtaggcgcgctccaatcacaaggagtacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 104 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggaaccgtggccacaaaccttcagagtgcccaagcacaggcgcaggttggcgctctacaaagtcaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 105 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggagtggtggccacaaaccaccagagtgccacagcacaggcgcaggccggcgccttgcaatcacaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 106 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcccatggagtagtggccatgaaccaccagagtgcccaagcacaggcgagcgtaggcgcccttcaatctcaaggagtccttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 107 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaacttttagagacaacgtggatgcggacaaagtcatgataaccaacgaagaagaaattaaaactactaacccggtagcaacggagtcctatggaaccgtggccacaaaccaccagagtgcccaagcacaggcgcaggtaggcgcgctgcaaagccaaggagtacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 108 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtggtatggagtggtggccacaaaccaccagagttcccaaacacaggcgcaggttggcgctttgcaaagccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 109 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaaaagacaacgtggatgcggacaaagtcatgataaccaacgaagaagaaattaaaactactaacccggtagcaacggagtcctatggagtagtggccacaaaccaccagagtgcccaaagtcaggcgcagagcggcgccctgcaatcccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 110 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactataagagacaacgtggatgcggacaaagtcatgataaccaacgaagaagaaattaaaactactaacccggtagcaacggagtcctatggagtggtggccacaaaccaccagagtgcccaagcacaggcgcagagtggcgcgctgcaaagccaaggagtccttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 111 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactcatagagacaacgtggatgcggacaaagtcatgataaccaacgaagaagaaattaaaactactaacccggtagcaacggagtcctatggaaccgtggccacaaaccaccagagtgcccaagcacaggcgcaggtaggcgcgcttcaaagccaaggagtacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 112 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtccaaataaccaacgaagaagaaattaaaactactaacccggtagcaacggagtcctatggagtcgtggccataaaccaccagagtgcccaagcacaggcgcaggttggcgccctgcaatcacaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 113 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggagtagtggccacaaaccaccagagtgcccaagcacaggcgcaggttggcgcgttgcaaacccaaggagtacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 114 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtccactggagtcgtggccacaaaccaccagagtgcccaatcccaggcggcggtaggcgccctacaaagtcaaggagttcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 115 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggaactgtggccacaaacaatcagagtgcccaagcacaggcgcaggtaggcgcgttacaatctcaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 116 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggagtggtggccacaaaccaccagagtgccaatgcacaggcgcaggtcggcgcattgcaaactcaaggagtccttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 117 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggagtcgtggccacaaaccaccagagtgccaacgcacaggcgcaggcgggcgccctgcaatcccaaggagtgcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 118 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggaactgtggccacaaaccaccagagtgccaacacccaggcgaatgtgggcgccttgcaatcccaaggagtccttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 119 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggattagacaacgtggatgcggacaaagtcatgataaccaacgaagaagaaattaaaactactaacccggtagcaacggagtcctatggaaccgtggccacaaaccaccagagtgcccaagcacaggcgcaggtgggcgcgctgcaatcgcaaggagttcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 120 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaacgtcatgataaccaacgaagaagaaattaaaactactaacccggtagcaacggagtcctatggagtagtggccacaaaccaccagagtgccaatgcacaggcgcaggccggcgcgctccaatcccaaggagtgcttccgggtatggtttggcagaacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 121 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggaacagtggccacaaaccaccagcagtcacaagcacaggcgcaggtgggcgctcttcaatctcaaggagtgcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 122 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggaaccgtggccacaaaccaccagagtgccaatacgcaggcggcggtgggcgctctccaatcccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 123 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggagttgtggccacaaaccaccagagtagccaagcacaggcgcagagcggcgcactacaatcacaaggagttcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 124 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggaacggtggccacaaaccaccagagtgccaatacacaggcgcaggttggcgcgcttcaatctcaaggagtacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 125 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactaatagagacaacgtggatgcggacaaagtcatgataaccaacgaagaagaaattaaaactactaacccggtagcaacggagtcctatggaacagtggccacaaaccaccagagtagtcaaacacaggcgcaggtcggcgcgctacaaagtcaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 126 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggaccttgtccagataaccaacgaagaagaaattaaaactactaacccggtagcaacggagtcctatggagtggtggccacaaaccaccagagtgcccaagcacaggcgcaggcgggcgcacttcaaagccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 127 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtggtatggaactgtgaatacaaaccaccagagtgcccaagcacaggcgcaggtcggcgcgctgcaatcacaaggagtacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 128 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggagtggtggccacaaacaatcagagtgcccaagcacaggcgcaggtaggcgctctacaaagtcaaggagtccttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 129 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcgccataaccaacgaagaagaaattaaaactactaacccggtagcaacggagtcctatggaactgtggccacaaaccaccagcagacccaagcacaggcgcaggtgggcgctctgcaatcacaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 130 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcactataaccaacgaagaagaaattaaaactactaacccggtagcaacggagtcctatggagtagtggccacaaaccaccagagttcgaacgcacaggcgcagagcggcgccctccaatcacaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 131 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcattataaccaacgaagaagaaattaaaactactaacccggtagcaacggagtcctatggaagcgtggccacaaaccaccagagtgcccaagcacaggcggccaccggctggctacaatcccaaggagctcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 132 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggatcagtggccacaaaccttcagagtgcccaagcacaggcgcaggttggctacctgcaaagccaaggagtccttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 133 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggatcggtggccacaaaccaccagagtgcccaagcacagcctcagaccggctggttacaatcccaaggagcacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 134 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacgtggtcctatggacaagtggccacaaacaaccagagtgcccaagcacaggcgcaggtaggcgcactacaatctcaaggagtacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 135 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacgtcttcctatggacaagtggccacaaacatgcagagtgcccaagcacaggcgcaggttggcgccttacaatctcaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 136 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAIVGALQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 137 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQVGALQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 138 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQVGWLQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 139 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQTGALQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 140 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAIVGWLQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 141 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAITGALQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 142 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAIVGALQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 143 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQVGWLQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 144 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQVGALQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 145 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAIVGWLQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 146 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAIVGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 147 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQVGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 148 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQVGWLQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 149 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQTGWLQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 150 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAITGWLQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 151 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAIVGWLQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 152 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAIVGAVQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 153 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAIVGWLQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 154 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAIVGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 155 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQTGAVQNQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 156 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAITGWLQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 157 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQTGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 158 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWLQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 159 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAIVGAVQNQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 160 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGALQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 161 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAITGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 162 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQVGALQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 163 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAITGWVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 164 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQTGWLQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 165 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQTGWLQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 166 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQVGWVQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 167 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAITGAVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 168 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQVGWLQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 169 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAITGAVQNQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 170 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWLQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 171 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAIVGAVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 172 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAITGWLQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 173 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAITGWLQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 174 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAIVGALQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 175 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQVGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 176 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQVGAVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 177 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 178 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAIVGAVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 179 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAIVGWVQNQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 180 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQTGALQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 181 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQVGAVQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 182 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQTGALQNQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 183 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 184 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQVGAVQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 185 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQVGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 186 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAITGALQNQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 187 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWLQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 188 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWLQNQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 189 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQVGALQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 190 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQVGALQNQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 191 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAITGWVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 192 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAIVGWVQNQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 193 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQVGALQNQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 194 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQTGAVQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 195 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 196 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQTGWVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 197 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAITGAVQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 198 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQVGAVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 199 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQVGAVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 200 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAIVGWVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 201 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAITGWVQNQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 202 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 203 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAIVGWVQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 204 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGAVQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 205 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAIVGWLQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 206 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAIVGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 207 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQVGWLQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 208 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQVGWLQNQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 209 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAITGWLQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 210 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAITGWLQNQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 211 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQTGWLQNQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 212 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAITGALQNQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 213 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAIVGALQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 214 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQVGWVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 215 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAITGAVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 216 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAIVGWVQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 217 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQTGWVQNQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 218 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAIVGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 219 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAITGAVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 220 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGAVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 221 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAITGALQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 222 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGALQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 223 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAIVGALQNQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 224 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAITGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 225 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAITGALQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 226 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAIVGAVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 227 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAITGALQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 228 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGALQNQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 229 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAITGAVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 230 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQVGWVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 231 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQVGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 232 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQTGAVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 233 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAITGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 234 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 235 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGAVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 236 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGAVQNQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 237 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQVGAVQNQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 238 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQVGWVQNQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 239 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAITGAVQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 240 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAITGWVQNQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 241 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQTGAVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 242 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAIVGWVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 243 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAITGAVQNQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 244 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAITGWVQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 245 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAIVGAVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 246 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQVGWVQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 247 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAQTGWVQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 248 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAITGWVQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 249 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQVGAVQSQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 250 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQVGWLQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 251 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAITGALQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 252 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAIVGWLQNQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 253 - DNA, 798 bp, synthetic construct
  source 1..798: mol_type = other DNA
ccaacctgaaaaaaagtgatttcaggcaggtgctccaggtaattaaacattaataccccaccaaccaaccatcccttaaacccttacctcttgctcagctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcacaatgccaggcgggccatttaccgtcattgacgtcaatagggggcgtacttggcatatgatacacttgatgtactgccaagtgggcagtttaccgtaaatactccacccattgacgtcaatggaaagtccctattggcgttactattgacgtcaatgggcgggggtcgttgggcggtcagccaggcgggccatttaccgtaagttatgtaacg

SEQ ID NO: 254 - DNA, 448 bp, synthetic construct
  source 1..448: mol_type = other DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 255 - protein, 9 aa, synthetic construct
PLNGAVHLY

SEQ ID NO: 256 - protein, 7 aa, synthetic construct
IVMNSLK

SEQ ID NO: 257 - protein, 7 aa, synthetic construct
RDSPKGW

SEQ ID NO: 258 - protein, 7 aa, synthetic construct
YSTDVRM

SEQ ID NO: 259 - protein, 7 aa, synthetic construct
RESPRGL

SEQ ID NO: 260 - protein, 7 aa, synthetic construct
GNNTRSV

SEQ ID NO: 261 - protein, 7 aa, synthetic construct
GNNTRDT

SEQ ID NO: 262 - protein, 7 aa, synthetic construct
TNSTRPV

SEQ ID NO: 263 - protein, 7 aa, synthetic construct
ASSLNIA

SEQ ID NO: 264 - protein, 35 aa, synthetic construct
SLEEEWAQVECEVYGRGCPSGSLDESFYDWFERQL

SEQ ID NO: 265 - protein, 96 aa, synthetic construct
TPFYPVGYTVKQPGTCGDGVLQPGEECDDGNPDVSDGCIDCHRAYCGDGYRHQGVEDCDGSDFGYLTCETYLPGSYGDLRCTQYCSIDSTPCRYFT

SEQ ID NO: 266 - protein, 4 aa, synthetic construct
RGDY

SEQ ID NO: 267 - protein, 4 aa, synthetic construct
RGDF

SEQ ID NO: 268 - skipped

SEQ ID NO: 269 - protein, 7 aa, synthetic construct
RGDLGLS

SEQ ID NO: 270 - protein, 7 aa, synthetic construct
RGDLSTP

SEQ ID NO: 271 - protein, 10 aa, synthetic construct
SNSRGDYNSL

SEQ ID NO: 272 - protein, 10 aa, synthetic construct
ENRRGDFNNT

SEQ ID NO: 273 - protein, 8 aa, synthetic construct
SRGDYNSL

SEQ ID NO: 274 - protein, 7 aa, synthetic construct
RGDYNSL

SEQ ID NO: 275 - protein, 6 aa, synthetic construct
RGDLST

SEQ ID NO: 276 - protein, 7 aa, synthetic construct
RGDYVGL

SEQ ID NO: 277 - protein, 7 aa, synthetic construct
RGDAVGV

SEQ ID NO: 278 - protein, 4 aa, synthetic construct
GGGS

(19) United States
(12) Patent Application Publication
(10) Pub. No.: US 2026/0207784 A1
(43) Pub. Date: Jul. 23, 2026

(54) CAPSID POLYPEPTIDES AND METHODS OF USE THEREOF

(71) Applicant: DYNO THERAPEUTICS, INC., Watertown, MA (US)

(72) Inventors: Ina Chen, Sommerville, MA (US); Jerrah Holth, Sudbury, MA (US); Hanna Levitin, Cambridge, MA (US); Kathy S. Lin, Sommerville, MA (US)

(73) Assignee: DYNO THERAPEUTICS, INC., Watertown, MA (US)

(21) Appl. No.: 19/163,772

(22) PCT Filed: Mar. 8, 2024
(86) PCT No.: PCT/US2024/019023

§ 371 Date: Sep. 9, 2025

Related U.S. Application Data
(60) Provisional application No. 63/489,598, filed on Mar. 10, 2023.
(60) Provisional application No. 63/495,614, filed on Apr. 12, 2023.
(60) Provisional application No. 63/535,725, filed on Aug. 31, 2023.

(51) Int. Cl. A61K 48/00; C07K 14/005; C12N 15/86

(52) U.S. Cl. CPC A61K 48/0058; C07K 14/005; C12N 15/86; C12N 2750/14122; C12N 2750/14143; C12N 2750/14145; C12N 2750/14152

(57) ABSTRACT

The disclosure is directed in part to dependoparvovirus capsid polypeptides that can be used to deliver payloads.

### 1. CROSS-REFERENCE TO RELATED APPLICATIONS

This application claims the priority benefit of U.S. provisional application No. 63/489,598, filed Mar. 10, 2023, U.S. provisional application No. 63/495,614, filed Apr. 12, 2023, and U.S. provisional application No. 63/535,725, filed Aug. 31, 2023, the contents of which are incorporated herein in their entireties by reference thereto.

### 2. SEQUENCE LISTING

The instant application contains a Sequence Listing which has been submitted electronically in XML format and is hereby incorporated by reference in its entirety. Said XML Sequence Listing, created on Feb. 29, 2024, is named DYN-001WO_SL.xml and is 525,148 bytes in size.

### 3. BACKGROUND

Dependoparvoviruses, e.g., adeno-associated dependoparvoviruses, e.g., adeno-associated viruses (AAVs), are of interest as vectors for delivering various payloads to cells, including in human subjects.

### 4. SUMMARY

The present disclosure relates, in part, to improved dependoparvovirus capsid polypeptides, such as VP1, VP2 and/or VP3 capsid polypeptides, methods of producing a dependoparvovirus comprising capsid polypeptides, compositions for use in the same, as well as viral particles produced by the same. In certain aspects, the present disclosure relates to viral particles comprising the improved dependoparvovirus capsid polypeptides, with increased central nervous system (CNS) biodistribution and/or transduction as compared to viral particles, e.g., without the mutations in the improved dependoparvovirus capsid polypeptides. In certain aspects, the present disclosure relates to viral particles comprising the improved dependoparvovirus capsid polypeptides, with increased skeletal muscle biodistribution and/or transduction as compared to viral particles, e.g., without the mutations in the improved dependoparvovirus capsid polypeptides.

Accordingly, the present disclosure provides a capsid polypeptide described herein. In some embodiments, the capsid polypeptide comprises an amino acid sequence having at least 70%, 75%, 80%, 85%, 90%, 91%, 92%, 93%, 94%, 95%, 96%, 97%, 98%, 99% or 100% identity to a VP1 polypeptide of any one of SEQ ID NO:12 to SEQ ID NO:73 or to a VP2 or VP3 portion thereof. Typically, a capsid polypeptide of the disclosure comprises a mutation at the position corresponding to V596 (e.g., V596L) and/or N598 (e.g., N598S or N598T) as compared to a capsid polypeptide of SEQ ID NO:1, and optionally further comprises a mutation at one or more of Q579 (e.g., Q579S, S579T or Q579V), T593 (e.g., T593A, T593R, T593S or T593V), W595 (e.g., W595A or W595Y), 1601 (e.g., 1601A or 1601V) and Q592 (e.g., Q592A, Q5921, Q592N, or Q592S) as compared to a capsid polypeptide of SEQ ID NO:1. In some embodiments, the capsid polypeptide comprises a mutation at V596, N598 and Q579 as compared to a capsid polypeptide of SEQ ID NO:1. In some embodiments, the capsid polypeptide comprises a mutation at V596, N598 and T593 as compared to a capsid polypeptide of SEQ ID NO:1. In some embodiments, the capsid polypeptide comprises a mutation at V596, N598, Q579 and T593 as compared to a capsid polypeptide of SEQ ID NO:1. In further embodiments, the capsid polypeptide comprises a mutation at any of the foregoing combinations of position as compared to a capsid polypeptide of SEQ ID NO:1, together with a mutation at W595, 1601, Q592 or any combination of two or all three of W595, 1601 and Q592 as compared to a capsid polypeptide of SEQ ID NO:1. In some embodiments, the percentage sequence identity is calculated excluding any targeting peptide sequence insertion(s) in the capsid polypeptide sequence. In other embodiments, the percentage sequence identity is calculated including any targeting peptide sequence insertion(s) in the capsid polypeptide sequence. Additional exemplary capsid polypeptides are disclosed in Section 5.2 and numbered embodiments 1 to 531.

The present disclosure further provides a nucleic acid comprising a nucleotide sequence encoding a capsid polypeptide as provided for herein, e.g., a capsid polypeptide disclosed in Section 5 2 or any one of numbered embodiments 1 to 531. In some embodiments, the nucleic acid molecule comprises a nucleotide sequence of any one of SEQ ID NO:74 to SEQ ID NO:135, respectively, a fragment thereof (e.g., a fragment thereof encoding a VP2 or VP3 polypeptide), or a variant of any of the foregoing having at least 70%, 75%, 80%, 85%, 90%, 91%, 92%, 93%, 94%, 95%, 96%, 97%, 98%, 99%, or 100% sequence identity thereto. In some embodiments, the percentage sequence identity is calculated excluding any nucleotide sequence(s) encoding targeting peptide sequence insertion(s). In some embodiments, the percentage sequence identity is calculated including any nucleotide sequence(s) encoding targeting peptide sequence insertion(s). In some embodiments, the nucleic acid is a vector, e.g., a plasmid. Exemplary nucleic acids are disclosed in Section 5.2 and numbered embodiments 532 to 551.

The present disclosure further provides a dependoparvovirus particle comprising a capsid polypeptide, a capsid polypeptide disclosed in Section 5.2 or any one of numbered embodiments 1 to 531 and/or a nucleic acid described herein, e.g., a nucleic acid disclosed in Section 5.2 or any one of numbered embodiments 532 to 551 or a nucleic acid comprising a transgene/payload as disclosed in Section 5.7. In some embodiments, the dependoparvovirus is an adeno-associated dependoparvovirus (AAV). In some embodiments, the AAV is AAV9, e.g., a variant AAV9. Exemplary virus particles are disclosed in Section 5.3 and numbered embodiments 552 to 736. In some embodiments, the virus particles have one or more characteristics disclosed in Section 5.4 and numbered embodiments 570 to 736.

In some embodiments, the disclosure is directed, in part, to a cell, cell-free system, or other translation system comprising a nucleic acid or vector described herein, e.g., comprising a sequence encoding a capsid polypeptide having one or more mutations described herein, for example a capsid polypeptide disclosed in Section 5.2 or any one of numbered embodiments 1 to 531. In some embodiments, the cell, cell-free system, or other translation system comprises a dependoparvovirus particle described herein, e.g., wherein the particle comprises a nucleic acid comprising a sequence encoding a capsid polypeptide, e.g., a capsid polypeptide disclosed in Section 5.2 or any one of numbered embodiments 1 to 531 and/or a nucleic acid described herein, e.g., a nucleic acid disclosed in Section 5.2 or any one of numbered embodiments 532 to 551 or a nucleic acid comprising a transgene as disclosed in Section 5.7. Exemplary cells, cell-free and other translation systems and their use to produce dependoparvovirus particles are disclosed in Section 5.5 and in numbered embodiments 737, 738, 1807 to 1812, and 1816 to 1827.

The present disclosure further provides methods of using a dependoparvovirus disclosed herein, e.g., for delivering a payload to a cell or treating a disease or condition in a subject. The methods typically comprise contacting the cell or administering to the subject a dependoparvovirus particle described herein in an amount effective to treat the disease or condition. Exemplary methods are disclosed in Section 5.6 and numbered embodiments 748 to 1323. The dependoparvovirus particles may be in the form of a composition, e.g., a pharmaceutical composition comprising the dependoparvovirus particles and a pharmaceutically acceptable carrier or excipient, for example as described in Section 5.8 and numbered embodiment 1813. The disclosure further provides compositions disclosed herein for use in treating a disease or condition in a subject and for use in the manufacture of a medicament for use in treating a disease or condition in a subject. Exemplary compositions for use are described in numbered embodiments 1814 and 1815.

Additional features, advantages and applications of the capsid polypeptides, nucleic acids, dependoparvovirus particles of the disclosure and methods of their production and use are more particularly described below.

### 5. BRIEF DESCRIPTION OF THE DRAWINGS

FIGS. 1A-1C. Illustration of exemplary AAV serotype alignments. Amino acids that are present only in VP1 polypeptides are in normal text; amino acids that are present only in VP1 and VP2 polypeptides are in bold; amino acids that are present in VP1, VP2 and VP3 polypeptides are underlined.

FIGS. 2A-2D. Genome maps of plasmids used in Example 2. A) Wild-type AAV9 rep cap plasmid. B) VAR-B1 rep cap plasmid. C) Packaging plasmid pertaining to the heterologous nucleic acid sequence packaged in the VAR-B1 capsid: ITR-containing plasmid encoding NLS-eGFP. D) Packaging plasmid pertaining to the heterologous nucleic acid sequence packaged in the wild-type AAV9 capsid: ITR-containing plasmid encoding NLS-mCherry.

FIGS. 3A-3B. Relative transduction (A) and biodistribution (B) for VAR-B1 (relative to wild-type AAV9) from the 2-capsid NHP experiment described in Example 2.

FIGS. 4A-4B. A) Representative images of VAR-B1 and wild-type AAV9 immunofluorescence in the hippocampus and cerebellum. In the hippocampus, the number of cells with VAR-B1 GFP expression is ~25 fold those with AAV9 mCherry expression. This was also found in the CA3 layer, where VAR-B1 GFP is visible in the CA3 pyramidal neurons showing co-staining with the neuronal marker NeuN. In the cerebellum, VAR-B1 GFP is expressed in more cells than AAV9 mCherry and has highest expression in the Purkinje cell layer. B) Representative images of VAR-B1 and wild-type AAV9 immunofluorescence in the cervical spinal cord, frontal cortex, and caudate. The number of cells with VAR-B1 GFP expression in the spinal cord is 9.4-fold AAV9 mCherry. VAR-B1-expressed GFP is also detected in some neurons (co-stain with NeuN), unlike AAV9 mCherry. In the frontal cortex and caudate, the total number of cells expressing VAR-B1 GFP is increased compared to AAV9 mCherry (15.9- and 4.6-fold increase) and this increases to 25 and 37-fold for neurons specifically (co-stain with NeuN).

FIG. 5. Representative images from in vitro cell transduction experiments showing transduction of VAR-B1 and WT AAV9 in both Primary Human Neurons and the Sh-sy5y cell line. Primary human neurons were treated with virus at 50K MOI and Sh-sy5y cells with virus at 100K MOI for both VAR-B1 and WT AAV9. Images were taken from fixed samples using a 20× objective on an EVOS M5000.

FIG. 6. Neuronal transduction rates of VAR-1, VAR-2, VAR-3, VAR-18, VAR-54, and VAR-B1. Transduction rates were normalized to cell abundance and capsid abundance in the test article (TA) for the aggregated cortex and basal ganglia regions.

### 5.1. Definitions

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

A, An, The: As used herein, the singular forms “a,” “an” and “the” include plural referents unless the context clearly dictates otherwise.

About, Approximately: As used herein, the terms “about” and “approximately” shall generally mean an acceptable degree of error for the quantity measured given the nature or precision of the measurements. Exemplary degrees of error are within 15 percent (%), typically, within 10%, and more typically, within 5% of a given value or range of values. Any disclosure herein of a value preceded by the term “about” or “approximately” is also a disclosure of the value per se. For example, disclosure of “about 10 μg/ml” is a disclosure of the value “10 μg/ml.”

CNS: As used herein, “CNS” means one or more regions of the central nervous system. In embodiments, the CNS includes one or more of: brain and spinal cord.

Corresponds to: As used herein, the term “corresponds to” as used in reference to a position in a sequence, such as an amino acid or nucleic acid sequence, can be used in reference to an entire capsid polypeptide or polynucleotide sequence, such as the full-length sequence of the capsid polypeptide that comprises a VP1, VP2, and VP3 polypeptide, or a nucleic acid molecule encoding the same. In some embodiments, the term “corresponds to” can be used in reference to a region or domain of the capsid polypeptide. For example, a position that corresponds to a position in the VP1 section of the reference capsid polypeptide can correspond to the VP1 portion of the polypeptide of the variant capsid polypeptide. Thus, when aligning the two sequences to determine whether a position corresponds to another position the full-length polypeptide can be used or domains (regions) can be used to determine whether a position corresponds to a specific position. In some embodiments, the region is the VP1 polypeptide. In some embodiments, the region is the VP2 polypeptide. In some embodiments, the region is the VP3 polypeptide. In some embodiments, when the reference polypeptide is the wild-type sequence (e.g., full-length or region) of a certain serotype of AAV, the variant polypeptide can be of the same serotype with a mutation made at such corresponding position as compared to the reference sequence (e.g., full-length or region). In some embodiments, the variant capsid polypeptide is a different serotype as compared to the reference sequence.

Dependoparvovirus capsid: As used herein, the term “dependoparvovirus capsid” refers to an assembled viral capsid comprising dependoparvovirus polypeptides. In some embodiments, a dependoparvovirus capsid is a functional dependoparvovirus capsid, e.g., is fully folded and/or assembled, is competent to infect a target cell, or remains stable (e.g., folded/assembled and/or competent to infect a target cell) for at least a threshold time.

Dependoparvovirus particle: As used herein, the term “dependoparvovirus particle” refers to an assembled viral capsid comprising dependoparvovirus polypeptides and a packaged nucleic acid, e.g., comprising a payload, one or more components of a dependoparvovirus genome (e.g., a whole dependoparvovirus genome), or both. In some embodiments, a dependoparvovirus particle is a functional dependoparvovirus particle, e.g., comprises a desired payload, is fully folded and/or assembled, is competent to infect a target cell, or remains stable (e.g., folded/assembled and/or competent to infect a target cell) for at least a threshold time.

Dependoparvovirus X particle/capsid: As used herein, the term “dependoparvovirus X particle/capsid” refers to a dependoparvovirus particle/capsid comprising at least one polypeptide or polypeptide encoding nucleic acid sequence derived from a naturally occurring dependoparvovirus X species or serotype. For example, a dependoparvovirus B particle refers to a dependoparvovirus particle comprising at least one polypeptide or polypeptide encoding nucleic acid sequence derived from a naturally occurring dependoparvovirus B sequence. Derived from, as used in this context, means having at least 70, 75, 80, 85, 90, 91, 92, 93, 94, 95, 96, 97, 98, 99, or 100% identity to the sequence in question. Correspondingly, an AAVX particle/capsid, as used herein, refers to an AAV particle/capsid comprising at least one polypeptide or polypeptide encoding nucleic acid sequence derived from a naturally occurring AAV X serotype. For example, an AAV9 particle refers to an AAV particle comprising at least one polypeptide or polypeptide encoding nucleic acid sequence derived from a naturally occurring AAV9 sequence. Sometimes, a dependoparvovirus X capsid is referred to as “Wild Type” or “wt” when such capsid comprises capsid polypeptides from a specified sequence identifier associated with such dependoparvovirus X capsid. Thus, for example, the terms wild-type AAV9 capsid or wtAAV9 capsid (or simply wtAAV9) are used interchangeably and refer to a capsid that comprises capsid polypeptides of SEQ ID NO:1 (e.g., a VP1 capsid of SEQ ID NO:1 and VP2 and VP3 portions thereof).

Edit Distance: Sequences disclosed herein may be described in terms of “edit distance.” The minimum number of sequence edits, i.e., additions, substitutions, or deletions of a single amino acid (for amino acid sequence) or a single nucleotide (for nucleotide sequences), which change one sequence into another sequence is the edit distance between the two sequences. The term “edit distance” is often used interchangeably with the term “Levenshtein distance.”

Exogenous As used herein, the term “exogenous” refers to a feature, sequence, or component present in a circumstance (e.g., in a nucleic acid, polypeptide, or cell) that does not naturally occur in said circumstance. For example, a nucleic acid sequence encoding a polypeptide can comprise an exogenous codon (e.g., codon encoding for an amino acid that does not naturally occur in that position, for example in a reference sequence), such as provided for herein. Use of the term exogenous in this fashion means that the codon in question at this position does not occur naturally, e.g., is not present in AAV9, e.g., is not present in SEQ ID NO:1. In some embodiments, the codon replaces an endogenous codon. In some embodiments, the exogenous codon is inserted into the nucleic acid sequence, for example, relative to a reference sequence. A person of skill will readily understand that a sequence (e.g., a codon) can be exogenous when provided in a particular sequence (e.g., that does not naturally comprise the codon at the site in question) but may not be exogenous in a second sequence (e.g., that does naturally comprise that particular codon at the site in question).

Functional: As used herein in reference to a polypeptide component of a dependoparvovirus capsid (e.g., Cap (e.g., VP1, VP2, and/or VP3) or Rep), the term “functional” refers to a polypeptide which provides at least 50, 60, 70, 80, 90, or 100% of the activity of a naturally occurring version of that polypeptide component (e.g., when present in a host cell). For example, a functional VP1 polypeptide can stably fold and assemble into a dependoparvovirus capsid (e.g., that is competent for packaging and/or secretion). As used herein in reference to a dependoparvovirus capsid or particle, “functional” refers to a capsid or particle comprising one or more of the following production characteristics: comprises a desired payload, is fully folded and/or assembled, is competent to infect a target cell, or remains stable (e.g., folded/assembled and/or competent to infect a target cell) for at least a threshold time.

Mutation Difference: As used herein with respect to a polypeptide sequence, means a single amino acid mutation (e.g., substitution, insertion or deletion) present in a subject polypeptide sequence, relative to a reference polypeptide sequence. In various embodiments, the reference polypeptide sequence is a polypeptide of any one of SEQ ID NO:1, SEQ ID NO:3, SEQ ID NO:7, SEQ ID NO:9, SEQ ID NO:11, SEQ ID NOs:12-73 and SEQ ID NOs:136-252, or a VP2 or VP3 portion thereof. In some embodiments, the reference polypeptide is any one of SEQ ID NO:1, SEQ ID NOs:12-73 and SEQ ID NOs:136-252, or a VP2 or VP3 portion thereof. In a preferred embodiment, the reference polypeptide is a polypeptide of SEQ ID NO:1. In various embodiments, the subject polypeptide is any one of SEQ ID NO:12 to SEQ ID NO:73, or a VP2 or VP3 portion thereof.

Mutation Set: As used herein, the term “mutation set” refers to the complete set of single amino acid mutations (substitutions, deletions and/or insertions) in a variant capsid polypeptide sequence (e.g., a polypeptide sequence of any one of SEQ ID NO:12 to SEQ ID NO:73, or a VP2 or VP3 portion thereof) relative to a reference sequence (e.g., a wild-type reference sequence). In some embodiments, the reference sequence is wild-type AAV9 VP1 capsid polypeptide (SEQ ID NO:1) or a VP2 or VP3 portion thereof. In some cases, part of the mutation set (i.e., more than one single amino acid mutation) is notated collectively; however, it will be understood that even when referred to in this way, the mutation set is a collection of single amino acid mutations. For example, an insertion of amino acid 1, 2, and 3 between amino acid N at position nn and amino acid W at position ww of a reference sequence may be notated as “Nnn_3aa_Www_123,” and it will be understood that each of amino acids 1, 2 and 3 represent separate single amino acid mutations within the mutation set. The mutation sets for certain variant capsid polypeptides described herein are found, for example, in Table 13 and Table 27. In some embodiments, a variant capsid polypeptide of the disclosure comprises a mutation set not consisting solely of a mutation set that is set forth in Table 13 (e.g., a mutation set present in a capsid polypeptide of any one of SEQ ID NOs:135-252).

Nucleic Acid: As used herein, in its broadest sense, the term “nucleic acid” refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid monomer (e.g., a nucleotide and/or nucleoside); in some embodiments, “nucleic acid” refers to an oligonucleotide chain comprising individual nucleic acid monomers or a longer polynucleotide chain comprising many individual nucleic acid monomers. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid is, comprises, or consists of one or more modified, synthetic, or non-naturally occurring nucleotides. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid has a nucleotide sequence that encodes a functional gene product such as an RNA or protein. In some embodiments, a nucleic acid is partly or wholly single stranded; in some embodiments, a nucleic acid is partly or wholly double stranded.

Or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

Percent Identity: Sequences disclosed herein may be described in terms of “percent identity” (% identity). For calculating percent identity between two amino acid sequences or two nucleic acid sequences, the two sequences to be compared are aligned using the EMBOSS Needle Pairwise Sequence Alignment software tool based on the Needleman and Wunsch algorithm (Needleman & Wunsch, 1970, J. Mol. Biol. 48(3):443-53) (available at www.ebi.ac.uk/Tools/psa/emboss_needle/) using the following parameters: Matrix: BLOSUM62 (for amino acid sequences) or DNAfull (for DNA sequences); Gap Open: 10; Gap Extend: 0.5; End Gap Penalty: false; End Gap Open: 10; and End Gap Extend: 0.5. Percent identity is determined by dividing the number of amino acid or nucleotide matches in the alignment by the length of the alignment and multiplying by 100. For example, if an alignment of two amino acid sequences has 95 matching amino acids and an alignment length of 100 amino acids, the two sequences have 95% identity.

When calculating percent identity of two capsid polypeptides, one or both of which contain(s) one or more targeting peptide insertions, percent identity can be determined without removing the targeting peptide insertion sequence(s) from the capsid polypeptide sequence(s) or, alternatively, percent identity can be determined after removing the targeting peptide insertion sequence(s) from the capsid polypeptide sequence(s). For example, if a first capsid polypeptide has an identical sequence to a second capsid polypeptide, except that the first capsid polypeptide has a 7-mer targeting peptide insertion, the two capsid polypeptides have less than 100% sequence identity when percent identity is determined without removal of the targeting peptide insertion sequence from the first capsid polypeptide sequence, whereas the two capsid polypeptides have 100% sequence identity when the targeting peptide insertion sequence is removed from the first capsid polypeptide sequence prior to calculating percent identity. References herein to percent identity of capsid polypeptides without mention of a targeting peptide refer to percent identity of the capsid polypeptides determined following removal of targeting polypeptide insertion sequence(s), if any, present in both capsid polypeptides, unless required otherwise by context. References herein to percent identity calculated “taking targeting peptide insertions into account” means that the percent identity is calculated without removal of targeting polypeptide insertion sequence(s), if any, present in both capsid polypeptides. References herein to percent identity calculated “without taking targeting peptide insertions into account” means that the percent identity is calculated following removal of targeting polypeptide insertion sequence(s), if any, present in both capsid polypeptides.

PNS: as used herein, “PNS” means one or more regions of the peripheral nervous system that does not include the CNS. In embodiments, the PNS includes dorsal root ganglia. In embodiments, the PNS includes sensory neurons and motor neurons.

Polypeptide, peptide, and protein: The terms “polypeptide,” “peptide” and “protein” are used interchangeably herein to refer to polymers of amino acids of any length. The polymer may be linear or branched, it may comprise modified amino acids, and it may be interrupted by non-amino acids.

Targeting Peptide: As used herein, a “targeting peptide” refers to a peptide inserted into, or attached to, a capsid polypeptide to alter the tropism of the capsid polypeptide. A targeting peptide can be inserted into an AAV capsid sequence for enhanced targeting to a desired cell-type, tissue, or organ, for example for enhanced targeting to the CNS. A targeting peptide is typically 3 to 20 amino acids in length, for example, 3 to 12 amino acids, 5 to 12 amino acids, 5 to 10 amino acids, or 7 to 10 amino acids in length.

Treating: As used herein, the term “treating a disease or condition” refers to treating a manifest disease or condition, for example, where the subject is already suffering from one or more symptoms of the disease or condition, or refers to treating a pre-manifest disease or condition, for example, where the subject is identified as having a disease or condition but is not yet exhibiting one or more symptoms of the disease or condition. Pre-manifest conditions may be identified by, for example, genetic testing.

Variant: As used herein, a “variant capsid polypeptide” refers to a polypeptide that differs from a reference sequence (e.g., SEQ ID NO:1, SEQ ID NO:3, SEQ ID NO:7, SEQ ID NO:9 or SEQ ID NO:11, preferably SEQ ID NO:1, or sequence subunit thereof such as a VP2 or VP3 portion thereof). The variant capsid polypeptide can, for example, comprise a mutation (e.g., substitution, deletion, or insertion). In some embodiments, the variant is about, or at least, 70%, 75%, 80%, 85%, 86%, 87%, 88%, 89%, 90%, 91%, 92%, 93%, 94%, 95%, 96%, 97%, 98%, or 99% identical to the reference sequence. It will be clear to the skilled artisan from this disclosure that any capsid polypeptide, for example any capsid polypeptide disclosed herein, for example, a capsid polypeptide of any one of SEQ ID NO:12 to SEQ ID NO:73, is a variant capsid polypeptide with respect to another capsid polypeptide having a different amino acid sequence, e.g., another capsid polypeptide with a reference sequence as set forth above. Thus, the term “variant capsid polypeptide” herein means, and is used interchangeably with, “capsid polypeptide,” and does not require any comparison to a specific reference sequence. In some embodiments, the reference sequence is a polypeptide comprising SEQ ID NO:1. In some embodiments, the reference sequence comprises or consists of a VP1, VP2 or VP3 polypeptide, e.g., of SEQ ID NO:1. In some contexts used herein, the term “variant” refers to a virus particle that includes a variant capsid polypeptide, e.g., described herein.

### 5.2. Capsid Polypeptides and Nucleic Acids Encoding the Same

The disclosure is directed, in part, to a variant capsid polypeptide, and to a nucleic acid comprising a sequence encoding the variant capsid polypeptide, wherein the variant capsid polypeptide comprises a mutation (insertion, deletion, or substitution) as compared to the wild-type sequence. In some embodiments, the wild-type sequence is SEQ ID NO:1. The disclosure is directed, in part, to a variant capsid polypeptide comprising SEQ ID NO:1 with one or more mutations as compared to SEQ ID NO:1, and nucleic acid molecules encoding the variant capsid polypeptide. The mutation can be, for example, an insertion, deletion, or substitution as compared to the wild-type sequence. In some embodiments, the wild-type sequence is SEQ ID NO:1.

In some embodiments, the variant capsid polypeptide comprises a mutation that corresponds to a position of a mutation present in any one of SEQ ID NO:12 to SEQ ID NO:73 as compared to SEQ ID NO:1.

In some embodiments, the disclosure provides a variant capsid polypeptide (and nucleic acids encoding said variant capsid polypeptide) that comprises at least 1 of the mutation differences associated with any variant capsid polypeptide of SEQ ID NO:12 to SEQ ID NO:73 or comprises at least 1 mutation which corresponds to a mutation difference associated with any variant capsid polypeptide of SEQ ID NO:12 to SEQ ID NO:73 In some embodiments, the disclosure provides a variant capsid polypeptide (and nucleic acids encoding said capsid polypeptide) that comprises at least 2 mutation differences associated with any variant capsid polypeptide of SEQ ID NO:12 to SEQ ID NO:73 or comprises at least 2 mutations which corresponds to 2 mutation differences associated with any variant capsid polypeptide of SEQ ID NO:12 to SEQ ID NO:73. In some embodiments, the disclosure provides a variant capsid polypeptide (and nucleic acids encoding said capsid polypeptide) that comprises at least 3 mutation differences associated with any variant capsid polypeptide of SEQ ID NO:12 to SEQ ID NO:73 or comprises at least 3 mutations which corresponds to 3 mutation differences associated with any variant capsid polypeptide of SEQ ID NO:12 to SEQ ID NO:73. In some embodiments, the disclosure provides a variant capsid polypeptide (and nucleic acids encoding said capsid polypeptide) that comprises at least 4 mutation differences associated with any variant capsid polypeptide of SEQ ID NO:12 to SEQ ID NO:73 or comprises at least 4 mutations which corresponds to 4 mutation differences associated with any variant capsid polypeptide of SEQ ID NO:12 to SEQ ID NO:73. In some embodiments, the disclosure provides a variant capsid polypeptide (and nucleic acids encoding said capsid polypeptide) that comprises at least 5 mutation differences associated with any variant capsid polypeptide of SEQ ID NO:12 to SEQ ID NO:73 or comprises at least 5 mutations which corresponds to 5 mutation differences associated with any variant capsid polypeptide of SEQ ID NO:12 to SEQ ID NO:73. In some embodiments, the disclosure provides a variant capsid polypeptide (and nucleic acids encoding said capsid polypeptide) that comprises at least 6 mutation differences associated with any variant capsid polypeptide of SEQ ID NO:12 to SEQ ID NO:73 or comprises at least 6 mutations which corresponds to 6 mutation differences associated with any variant capsid polypeptide of SEQ ID NO:12 to SEQ ID NO:73. In some embodiments, the disclosure provides a variant capsid polypeptide (and nucleic acids encoding said capsid polypeptide) that comprises at least 7 mutation differences associated with any variant capsid polypeptide of SEQ ID NO:12 to SEQ ID NO:73 or comprises at least 7 mutations which corresponds to 7 mutation differences associated with any variant capsid polypeptide of SEQ ID NO:12 to SEQ ID NO:73. In some embodiments, the disclosure provides a variant capsid polypeptide (and nucleic acids encoding said capsid polypeptide) that comprises at least 8 mutation differences associated with any variant capsid polypeptide of SEQ ID NO:12 to SEQ ID NO:73 or comprises at least 8 mutations which corresponds to 8 mutation differences associated with any variant capsid polypeptide of SEQ ID NO:12 to SEQ ID NO:73. In some embodiments, the disclosure provides a variant capsid polypeptide (and nucleic acids encoding said capsid polypeptide) that comprises at least 9 mutation differences associated with any variant capsid polypeptide of SEQ ID NO:12 to SEQ ID NO:73 or comprises at least 9 mutations which corresponds to 9 mutation differences associated with any variant capsid polypeptide of SEQ ID NO:12 to SEQ ID NO:73. In some embodiments, the disclosure provides a variant capsid polypeptide (and nucleic acids encoding said capsid polypeptide) that comprises at least 10 mutation differences associated with any variant capsid polypeptide of SEQ ID NO:12 to SEQ ID NO:73 or comprises at least 10 mutations which corresponds to 10 mutation differences associated with any variant capsid polypeptide of SEQ ID NO:12 to SEQ ID NO:73.

Mutations associated with VAR-1 to VAR-62 (corresponding to a capsid polypeptide of SEQ ID NO:12 to SEQ ID NO:73, respectively) are shown in Table 1 in relation to a VP1 polypeptide of SEQ ID NO:1.

TABLE 1

| | | | | | | | | | | | | | | | | | | | | | | | | | | | | Edit |
|---|---|---|---|---|---|---|---|---|---|---|---|---|---|---|---|---|---|---|---|---|---|---|---|---|---|---|---|---|
| | | | | | | | | | | | | | | | | | | | | | | | | | | | | distance |
| | | | | | | | | | | | | | | | | | | | | | | | | | | | | to |
| | | | | | | | | | | | | | | | | | | | | | | | | | | | | SEQ ID |
| VAR# | G549 | R550 | D551 | V553 | K557 | M559 | 1560 | T561 | E575 | S576 | Y577 | Q579 | A581 | T582 | H584 | S586 | A587 | Q588 | A589 | A591 | Q592 | T593 | W595 | V596 | N598 | 1601 | D609 | NO: 1 |
| 1 | _{—} | _{—} | _{—} | _{—} | _{—} | _{—} | _{—} | _{—} | _{—} | _{—} | _{—} | V | _{—} | _{—} | _{—} | _{—} | _{—} | _{—} | T | _{—} | _{—} | A | A | L | S | V | _{—} | 7 |
| 2 | _{—} | _{—} | M | _{—} | _{—} | _{—} | _{—} | _{—} | _{—} | _{—} | _{—} | T | _{—} | _{—} | _{—} | _{—} | _{—} | _{—} | _{—} | _{—} | S | V | A | L | S | V | _{—} | 8 |
| 3 | _{—} | _{—} | _{—} | _{—} | _{—} | _{—} | _{—} | _{—} | _{—} | _{—} | _{—} | V | _{—} | _{—} | _{—} | _{—} | _{—} | S | _{—} | _{—} | _{—} | A | A | L | S | V | _{—} | 7 |
| 4 | Y | _{—} | _{—} | _{—} | _{—} | _{—} | _{—} | _{—} | _{—} | _{—} | _{—} | T | _{—} | _{—} | _{—} | _{—} | _{—} | _{—} | _{—} | _{—} | _{—} | V | A | L | S | V | _{—} | 7 |
| 5 | _{—} | _{—} | _{—} | _{—} | _{—} | S | _{—} | _{—} | _{—} | _{—} | _{—} | T | _{—} | _{—} | I | _{—} | _{—} | _{—} | _{—} | _{—} | I | V | A | L | S | _{—} | _{—} | 8 |
| 6 | _{—} | _{—} | _{—} | _{—} | _{—} | N | _{—} | _{—} | _{—} | _{—} | _{—} | V | _{—} | _{—} | _{—} | _{—} | _{—} | _{—} | _{—} | _{—} | _{—} | S | A | L | S | V | _{—} | 7 |
| 7 | _{—} | _{—} | _{—} | _{—} | _{—} | _{—} | _{—} | _{—} | _{—} | _{—} | _{—} | V | _{—} | _{—} | _{—} | _{—} | _{—} | G | _{—} | _{—} | _{—} | S | A | L | S | V | _{—} | 7 |
| 8 | _{—} | _{—} | _{—} | _{—} | _{—} | _{—} | _{—} | _{—} | _{—} | _{—} | H | V | C | _{—} | _{—} | _{—} | _{—} | _{—} | _{—} | _{—} | _{—} | V | A | L | S | V | _{—} | 8 |
| 9 | _{—} | _{—} | _{—} | _{—} | _{—} | _{—} | L | _{—} | _{—} | _{—} | _{—} | V | _{—} | _{—} | _{—} | _{—} | _{—} | _{—} | T | _{—} | _{—} | A | A | L | S | _{—} | _{—} | 7 |
| 10 | _{—} | _{—} | _{—} | _{—} | _{—} | A | _{—} | _{—} | _{—} | _{—} | _{—} | V | _{—} | _{—} | M | _{—} | _{—} | _{—} | _{—} | _{—} | S | V | A | L | S | V | _{—} | 9 |
| 11 | _{—} | _{—} | _{—} | _{—} | _{—} | _{—} | _{—} | _{—} | _{—} | _{—} | _{—} | V | _{—} | _{—} | _{—} | _{—} | _{—} | N | _{—} | _{—} | _{—} | A | A | L | S | _{—} | _{—} | 6 |
| 12 | _{—} | _{—} | _{—} | _{—} | _{—} | V | _{—} | _{—} | _{—} | _{—} | _{—} | V | _{—} | _{—} | _{—} | _{—} | _{—} | _{—} | _{—} | _{—} | _{—} | S | A | L | S | V | _{—} | 7 |
| 13 | _{—} | _{—} | _{—} | _{—} | _{—} | _{—} | Q | _{—} | _{—} | _{—} | _{—} | T | _{—} | _{—} | _{—} | _{—} | _{—} | _{—} | _{—} | _{—} | _{—} | V | A | L | S | V | _{—} | 7 |
| 14 | _{—} | _{—} | _{—} | _{—} | _{—} | _{—} | _{—} | _{—} | _{—} | _{—} | _{—} | T | _{—} | _{—} | _{—} | _{—} | _{—} | N | T | _{—} | S | V | A | L | S | V | _{—} | 9 |
| 15 | _{—} | _{—} | _{—} | _{—} | _{—} | Q | _{—} | _{—} | _{—} | _{—} | _{—} | V | _{—} | _{—} | Q | _{—} | _{—} | _{—} | _{—} | _{—} | _{—} | V | A | L | S | V | _{—} | 8 |
| 16 | _{—} | _{—} | _{—} | _{—} | _{—} | _{—} | _{—} | _{—} | _{—} | _{—} | _{—} | V | _{—} | _{—} | _{—} | _{—} | _{—} | _{—} | _{—} | _{—} | I | R | A | L | S | _{—} | _{—} | 6 |
| 17 | _{—} | _{—} | _{—} | _{—} | _{—} | C | _{—} | _{—} | _{—} | _{—} | _{—} | V | _{—} | _{—} | _{—} | _{—} | _{—} | _{—} | _{—} | _{—} | _{—} | A | A | L | S | _{—} | _{—} | 6 |
| 18 | T | _{—} | _{—} | _{—} | _{—} | _{—} | _{—} | _{—} | _{—} | _{—} | _{—} | T | _{—} | _{—} | _{—} | _{—} | _{—} | _{—} | _{—} | _{—} | _{—} | V | A | L | S | V | _{—} | 7 |
| 19 | _{—} | _{—} | _{—} | _{—} | _{—} | _{—} | _{—} | _{—} | _{—} | _{—} | _{—} | V | _{—} | I | N | _{—} | _{—} | _{—} | _{—} | _{—} | _{—} | V | A | L | S | _{—} | _{—} | 7 |
| 20 | A | _{—} | _{—} | _{—} | _{—} | _{—} | _{—} | _{—} | _{—} | _{—} | _{—} | V | _{—} | _{—} | _{—} | _{—} | _{—} | _{—} | _{—} | _{—} | _{—} | S | A | L | S | V | _{—} | 7 |
| 21 | _{—} | _{—} | _{—} | _{—} | _{—} | _{—} | _{—} | _{—} | _{—} | _{—} | T | V | _{—} | _{—} | _{—} | _{—} | _{—} | _{—} | _{—} | _{—} | S | V | A | L | S | V | _{—} | 9 |
| 22 | _{—} | _{—} | _{—} | _{—} | _{—} | _{—} | _{—} | _{—} | _{—} | _{—} | _{—} | V | _{—} | _{—} | _{—} | _{—} | _{—} | _{—} | S | _{—} | _{—} | A | A | L | S | _{—} | _{—} | 7 |
| 23 | _{—} | _{—} | _{—} | _{—} | _{—} | _{—} | _{—} | S | _{—} | _{—} | _{—} | V | _{—} | _{—} | I | _{—} | _{—} | _{—} | _{—} | _{—} | S | V | A | L | S | V | _{—} | 9 |
| 24 | _{—} | _{—} | _{—} | _{—} | _{—} | _{—} | _{—} | _{—} | _{—} | _{—} | _{—} | V | _{—} | _{—} | I | _{—} | _{—} | _{—} | _{—} | _{—} | S | V | A | L | S | V | _{—} | 8 |
| 25 | _{—} | _{—} | _{—} | _{—} | _{—} | _{—} | _{—} | S | _{—} | _{—} | _{—} | V | _{—} | _{—} | _{—} | _{—} | _{—} | _{—} | _{—} | _{—} | _{—} | A | A | L | S | V | _{—} | 7 |
| 26 | _{—} | _{—} | _{—} | _{—} | _{—} | A | _{—} | _{—} | _{—} | _{—} | _{—} | V | _{—} | _{—} | _{—} | _{—} | _{—} | _{—} | _{—} | _{—} | S | V | A | L | T | V | _{—} | 8 |
| 27 | _{—} | _{—} | _{—} | _{—} | _{—} | _{—} | _{—} | _{—} | _{—} | _{—} | T | V | _{—} | _{—} | _{—} | _{—} | _{—} | _{—} | _{—} | _{—} | S | V | A | L | S | V | _{—} | 8 |
| 28 | _{—} | _{—} | _{—} | S | _{—} | _{—} | _{—} | _{—} | _{—} | _{—} | _{—} | T | _{—} | _{—} | _{—} | _{—} | _{—} | _{—} | _{—} | _{—} | _{—} | V | A | L | S | V | _{—} | 7 |
| 29 | _{—} | _{—} | _{—} | _{—} | _{—} | _{—} | _{—} | _{—} | _{—} | _{—} | _{—} | T | _{—} | _{—} | _{—} | _{—} | H | N | T | _{—} | I | V | A | L | S | V | _{—} | 10 |
| 30 | _{—} | _{—} | _{—} | _{—} | N | _{—} | _{—} | _{—} | _{—} | _{—} | _{—} | V | _{—} | _{—} | L | _{—} | _{—} | _{—} | T | _{—} | S | V | A | L | S | V | _{—} | 10 |
| 31 | _{—} | _{—} | _{—} | _{—} | _{—} | _{—} | _{—} | _{—} | _{—} | _{—} | _{—} | T | _{—} | _{—} | L | _{—} | _{—} | _{—} | _{—} | _{—} | _{—} | V | A | L | S | _{—} | _{—} | 6 |
| 32 | _{—} | _{—} | _{—} | _{—} | _{—} | _{—} | _{—} | _{—} | _{—} | _{—} | _{—} | V | _{—} | _{—} | _{—} | _{—} | _{—} | T | _{—} | _{—} | _{—} | A | A | L | S | _{—} | _{—} | 6 |
| 33 | _{—} | _{—} | _{—} | _{—} | _{—} | _{—} | _{—} | _{—} | _{—} | _{—} | H | V | _{—} | M | _{—} | _{—} | _{—} | _{—} | _{—} | _{—} | S | V | A | L | S | V | _{—} | 9 |
| 34 | F | _{—} | _{—} | _{—} | _{—} | _{—} | _{—} | _{—} | _{—} | _{—} | _{—} | T | _{—} | _{—} | _{—} | _{—} | _{—} | _{—} | _{—} | _{—} | _{—} | V | A | L | S | V | _{—} | 7 |
| 35 | _{—} | _{—} | _{—} | _{—} | _{—} | _{—} | _{—} | _{—} | _{—} | W | _{—} | V | _{—} | _{—} | _{—} | _{—} | S | _{—} | T | _{—} | _{—} | V | A | L | S | _{—} | _{—} | 8 |
| 36 | _{—} | K | _{—} | _{—} | _{—} | _{—} | _{—} | _{—} | _{—} | _{—} | _{—} | V | _{—} | _{—} | _{—} | _{—} | _{—} | _{—} | S | _{—} | _{—} | S | A | L | S | _{—} | _{—} | 7 |
| 37 | I | _{—} | _{—} | _{—} | _{—} | _{—} | _{—} | _{—} | _{—} | _{—} | _{—} | V | _{—} | _{—} | _{—} | _{—} | _{—} | _{—} | _{—} | _{—} | _{—} | S | A | L | S | V | _{—} | 7 |
| 38 | H | _{—} | _{—} | _{—} | _{—} | _{—} | _{—} | _{—} | _{—} | _{—} | _{—} | T | _{—} | _{—} | _{—} | _{—} | _{—} | _{—} | _{—} | _{—} | _{—} | V | A | L | S | V | _{—} | 7 |
| 39 | _{—} | _{—} | _{—} | _{—} | _{—} | Q | _{—} | _{—} | _{—} | _{—} | _{—} | V | _{—} | I | _{—} | _{—} | _{—} | _{—} | _{—} | _{—} | _{—} | V | A | L | S | _{—} | _{—} | 7 |
| 40 | _{—} | _{—} | _{—} | _{—} | _{—} | _{—} | _{—} | _{—} | _{—} | _{—} | _{—} | V | _{—} | _{—} | _{—} | _{—} | _{—} | _{—} | _{—} | _{—} | _{—} | V | A | L | T | V | _{—} | 6 |
| 41 | _{—} | _{—} | _{—} | _{—} | _{—} | _{—} | _{—} | _{—} | _{—} | _{—} | T | V | _{—} | _{—} | _{—} | _{—} | _{—} | _{—} | S | _{—} | A | V | A | L | S | V | _{—} | 9 |
| 42 | _{—} | _{—} | _{—} | _{—} | _{—} | _{—} | _{—} | _{—} | _{—} | _{—} | _{—} | T | _{—} | _{—} | N | _{—} | _{—} | _{—} | _{—} | _{—} | _{—} | V | A | L | S | _{—} | _{—} | 6 |
| 43 | _{—} | _{—} | _{—} | _{—} | _{—} | _{—} | _{—} | _{—} | _{—} | _{—} | _{—} | V | _{—} | _{—} | _{—} | _{—} | _{—} | N | _{—} | _{—} | _{—} | V | A | L | T | V | _{—} | 7 |
| 44 | _{—} | _{—} | _{—} | _{—} | _{—} | _{—} | _{—} | _{—} | _{—} | _{—} | _{—} | V | _{—} | _{—} | _{—} | _{—} | _{—} | _{—} | _{—} | _{—} | _{—} | A | A | L | S | V | _{—} | 7 |
| 45 | _{—} | _{—} | _{—} | _{—} | _{—} | _{—} | _{—} | _{—} | _{—} | _{—} | _{—} | T | _{—} | _{—} | _{—} | _{—} | _{—} | N | T | _{—} | _{—} | V | A | L | S | V | _{—} | 9 |
| 46 | _{—} | L | _{—} | _{—} | _{—} | _{—} | _{—} | _{—} | _{—} | _{—} | _{—} | T | _{—} | _{—} | _{—} | _{—} | _{—} | _{—} | _{—} | _{—} | _{—} | V | A | L | S | V | _{—} | 7 |
| 47 | _{—} | _{—} | _{—} | _{—} | N | _{—} | _{—} | _{—} | _{—} | _{—} | _{—} | V | _{—} | _{—} | _{—} | _{—} | _{—} | N | _{—} | _{—} | _{—} | A | A | L | S | V | N | 9 |
| 48 | _{—} | _{—} | _{—} | _{—} | _{—} | _{—} | _{—} | _{—} | _{—} | _{—} | _{—} | T | _{—} | _{—} | _{—} | Q | S | _{—} | _{—} | _{—} | _{—} | V | A | L | S | V | _{—} | 8 |
| 49 | _{—} | _{—} | _{—} | _{—} | _{—} | _{—} | _{—} | _{—} | _{—} | _{—} | _{—} | T | _{—} | _{—} | _{—} | _{—} | _{—} | N | T | _{—} | A | V | A | L | S | _{—} | _{—} | 8 |
| 50 | _{—} | _{—} | _{—} | _{—} | _{—} | _{—} | _{—} | _{—} | _{—} | _{—} | _{—} | V | _{—} | _{—} | _{—} | _{—} | S | _{—} | _{—} | _{—} | _{—} | S | A | L | S | V | _{—} | 7 |
| 51 | _{—} | _{—} | _{—} | _{—} | _{—} | _{—} | _{—} | _{—} | _{—} | _{—} | _{—} | T | _{—} | _{—} | _{—} | _{—} | _{—} | N | T | _{—} | _{—} | V | A | L | S | V | _{—} | 8 |
| 52 | N | _{—} | _{—} | _{—} | _{—} | _{—} | _{—} | _{—} | _{—} | _{—} | _{—} | T | _{—} | _{—} | _{—} | _{—} | S | _{—} | T | _{—} | _{—} | V | A | L | S | _{—} | _{—} | 8 |
| 53 | _{—} | _{—} | _{—} | _{—} | L | Q | _{—} | _{—} | _{—} | _{—} | _{—} | V | _{—} | _{—} | _{—} | _{—} | _{—} | _{—} | _{—} | _{—} | _{—} | A | A | L | S | _{—} | _{—} | 7 |
| 54 | _{—} | _{—} | _{—} | _{—} | _{—} | _{—} | _{—} | _{—} | _{—} | W | _{—} | T | N | _{—} | _{—} | _{—} | _{—} | _{—} | _{—} | _{—} | _{—} | V | A | L | S | V | _{—} | 8 |
| 55 | _{—} | _{—} | _{—} | _{—} | _{—} | _{—} | _{—} | _{—} | _{—} | _{—} | _{—} | V | _{—} | _{—} | N | _{—} | _{—} | _{—} | _{—} | _{—} | _{—} | V | A | L | S | V | _{—} | 7 |
| 56 | _{—} | _{—} | _{—} | _{—} | _{—} | _{—} | _{—} | _{—} | _{—} | _{—} | _{—} | S | _{—} | _{—} | L | _{—} | _{—} | _{—} | _{—} | _{—} | _{—} | V | Y | L | S | V | _{—} | 7 |
| 57 | _{—} | _{—} | _{—} | _{—} | _{—} | I | _{—} | _{—} | _{—} | _{—} | _{—} | S | _{—} | _{—} | _{—} | _{—} | _{—} | _{—} | _{—} | _{—} | A | _{—} | _{—} | L | S | A | _{—} | 6 |
| 58 | _{—} | _{—} | _{—} | _{—} | _{—} | _{—} | _{—} | _{—} | _{—} | _{—} | _{—} | S | _{—} | _{—} | _{—} | _{—} | _{—} | _{—} | _{—} | P | _{—} | _{—} | _{—} | L | S | A | _{—} | 5 |
| 59 | _{—} | _{—} | _{—} | _{—} | _{—} | _{—} | _{—} | _{—} | W | _{—} | _{—} | _{—} | _{—} | _{—} | N | _{—} | _{—} | _{—} | _{—} | _{—} | _{—} | V | A | L | S | V | _{—} | 7 |
| 60 | _{—} | _{—} | _{—} | _{—} | _{—} | A | _{—} | _{—} | _{—} | _{—} | _{—} | T | _{—} | _{—} | _{—} | Q | T | _{—} | _{—} | _{—} | _{—} | V | A | L | S | _{—} | _{—} | 8 |
| 61 | _{—} | _{—} | _{—} | _{—} | _{—} | T | _{—} | _{—} | _{—} | _{—} | _{—} | V | _{—} | _{—} | _{—} | _{—} | S | N | _{—} | _{—} | _{—} | S | A | L | S | _{—} | _{—} | 8 |
| 62 | _{—} | _{—} | _{—} | _{—} | _{—} | _{—} | _{—} | _{—} | S | _{—} | _{—} | _{—} | _{—} | _{—} | M | _{—} | _{—} | _{—} | _{—} | _{—} | _{—} | V | A | L | S | _{—} | _{—} | 6 |

In some embodiments, the disclosure provides a variant capsid polypeptide (and nucleic acids encoding said capsid polypeptide) that comprises all of the mutation differences associated with any variant capsid polypeptide of SEQ ID NO:12 to SEQ ID NO:73 or comprises mutations which corresponds to all of the mutation differences associated with any variant capsid polypeptide of SEQ ID NO:12 to SEQ ID NO:73.

In any of the above aspects it will be understood that in variant capsid polypeptides described above where a number of mutation differences associated with or corresponding to the mutation differences of any variant capsid polypeptide of SEQ ID NO:12 to SEQ ID NO:73 is specified, the mutations may be chosen from any of the mutation differences associated with that variant capsid polypeptide. Thus, for example, with respect to the mutation differences of a variant having mutation differences #1, #2, #3, and #4 relative to a reference polypeptide, where a variant capsid polypeptide comprises 1 of the mutation differences, it is #1 or #2 or #3 or #4; likewise, where a variant capsid comprises 2 of the mutation differences, those two are #1 and #2, #1 and #3, #1 and #4, #2 and #3, #2 and #4, or #3 and #4; likewise, where the variant comprises 3 of the mutation differences, those 3 are #1 and #2 and #3, #1 and #2 and #4, #1 and #3 and #4, or #2 and #3 and #4; likewise, where the variant comprises all 4 of the mutation differences, those four are #1, #2, #3 and #4. It will be understood by the skilled artisan that all the possible combinations of numbers of mutation differences for each variant capsid polypeptide of any of SEQ ID NO:12 to SEQ ID NO:73 (up to the total number of mutation differences for that variant capsid polypeptide) can be generated using routine skill and each such table for each of SEQ ID NO:12 to SEQ ID NO:73 is incorporated herein in its entirety. Such tables can be generated, for example, using the “combinations” method from the “itertools” package in Python, such method is hereby incorporated by reference in its entirety.

In some embodiments, the variant capsid polypeptide comprises one or more mutation differences associated with a capsid polypeptide described herein, e.g., associated with any of SEQ ID NO:12 to SEQ ID NO:73, and has at least 80%, at least 85%, at least 90%, at least 91%, at least 92%, at least 93%, at least 94%, at least 95%, at least 96%, at least 97%, at least 98%, or at least 99% sequence identity to a reference AAV serotype, e.g., as described herein, e.g., to SEQ ID NO:1. In embodiments, the variant capsid polypeptide comprises one or more mutation differences associated with a capsid polypeptide described herein, e.g., associated with any of SEQ ID NO:12 to SEQ ID NO:73 or which correspond to one or more mutation differences associated with a capsid polypeptide described herein, e.g., associated with any of SEQ ID NO:12 to SEQ ID NO:73. In embodiments, the variant capsid polypeptide is, but for the mutation differences described in or corresponding to the mutation differences associated with a capsid polypeptide described herein, e.g., associated with any of SEQ ID NO:12 to SEQ ID NO:73, at least 90%, at least 95%, 96%, 97%, 98%, 99%, or 100% identical to a reference AAV serotype described herein. In embodiments, the variant capsid polypeptide described herein is, but for the mutation differences associated with a capsid polypeptide described herein, e.g., associated with any of SEQ ID NO:12 to SEQ ID NO:73, or which correspond to the mutation differences associated with a capsid polypeptide described herein, e.g., associated with any of SEQ ID NO:12 to SEQ ID NO:73, comprised within such variant capsid polypeptide, at least 90%, at least 95%, 96%, 97%, 98%, 99%, or 100% identical to a capsid polypeptide of SEQ ID NO:1 (e.g., a VP1, VP2 or VP3 sequence of SEQ ID NO:1). In embodiments, the variant capsid polypeptide described herein is, but for the mutation differences associated with a capsid polypeptide described herein, e.g., associated with any of SEQ ID NO:12 to SEQ ID NO:73, or which correspond to the mutation differences associated with a capsid polypeptide described herein, e.g., associated with any of SEQ ID NO:12 to SEQ ID NO:73, comprised within such variant capsid polypeptide, at least 90%, at least 95%, 96%, 97%, 98%, 99%, or 100% identical to a capsid polypeptide of SEQ ID NO:3 (e.g., a VP1, VP2 or VP3 sequence of SEQ ID NO:3). In embodiments, the variant capsid polypeptide described herein is, but for the mutation differences associated with any of SEQ ID NO:12 to SEQ ID NO:73, or which correspond to the mutation differences associated with any of SEQ ID NO:12 to SEQ ID NO:73, comprised within such variant capsid polypeptide, at least 90%, at least 95%, 96%, 97%, 98%, 99%, or 100% identical to a capsid polypeptide of SEQ ID NO:5 (e.g., a VP1, VP2 or VP3 sequence of SEQ ID NO:5). In embodiments, the variant capsid polypeptide described herein is, but for the mutation differences associated with any of SEQ ID NO:12 to SEQ ID NO:73, or which correspond to the mutation differences associated with any of SEQ ID NO:12 to SEQ ID NO:73, comprised within such variant capsid polypeptide, at least 90%, at least 95%, 96%, 97%, 98%, 99%, or 100% identical to a capsid polypeptide of SEQ ID NO:7 (e.g., a VP1, VP2 or VP3 sequence of SEQ ID NO:7). In embodiments, the variant capsid polypeptide described herein is, but for the mutation differences associated with any of SEQ ID NO:12 to SEQ ID NO:73, or which correspond to the mutation differences associated with any of SEQ ID NO:12 to SEQ ID NO:73, comprised within such variant capsid polypeptide, at least 90%, at least 95%, 96%, 97%, 98%, 99%, or 100% identical to a capsid polypeptide of SEQ ID NO:9 (e.g., a VP1, VP2 or VP3 sequence of SEQ ID NO:9). In embodiments, the variant capsid polypeptide described herein is, but for the mutation differences associated with any of SEQ ID NO:12 to SEQ ID NO:73, or which correspond to the mutation differences associated with any of SEQ ID NO:12 to SEQ ID NO:73, comprised within such variant capsid polypeptide, at least 90%, at least 95%, 96%, 97%, 98%, 99%, or 100% identical to a capsid polypeptide of SEQ ID NO:1 1 (e.g., a VP1, VP2 or VP3 sequence of SEQ ID NO:1).

In some embodiments, a variant capsid polypeptide is provided that comprises a variant capsid polypeptide that is at least 70%, 75%, 80%, 85%, 90%, 91%, 92%, 93%, 94%, 95%, 96%, 97%, 98%, 99%, or 100% identical to a variant capsid polypeptide as provided herein.

In some embodiments, a variant capsid polypeptide is provided that comprises a variant capsid polypeptide that is at least 70%, 75%, 80%, 85%, 90%, 91%, 92%, 93%, 94%, 95%, 96%, 97%, 98%, 99%, or 100% identical (not including targeting peptide insertions) to a variant capsid polypeptide as provided herein.

In some embodiments, the variant capsid polypeptide comprises a VP1, VP2 VP3, or any combination thereof, that is each at least, or about, 95, 96, 97, 98 or 99% identical to a polypeptide of any one of SEQ ID NO:12 to SEQ ID NO:73, and optionally comprises at least one of, e.g., all of, the mutation differences of such SEQ ID NO:12 to SEQ ID NO:73.

In some embodiments, the variant capsid polypeptide comprises a VP1, VP2 VP3, or any combination thereof, that is each at least, or about, 95, 96, 97, 98 or 99% identical (not including target peptide insertions) to a polypeptide of any one of SEQ ID NO:12 to SEQ ID NO:73, and optionally comprises at least one of, e.g., all of, the mutation differences of such SEQ ID NO:12 to SEQ ID NO:73.

In some embodiments, the variant capsid polypeptide comprises a VP1, VP2, VP3, or any combination thereof, that each has about 1 to about 20 mutations as compared to a polypeptide of any one of SEQ ID NO:12 to SEQ ID NO:73, and optionally comprises at least one of, e.g., all of, the mutation differences of such SEQ ID NO:12 to SEQ ID NO:73.

In some embodiments, the variant capsid polypeptide comprises a VP1, VP2, VP3, or any combination thereof, that each has about 1 to about 10 mutations as compared to a polypeptide of any one of SEQ ID NO:12 to SEQ ID NO:73, and optionally comprises at least one of, e.g., all of, the mutation differences of such SEQ ID NO:12 to SEQ ID NO:73.

In some embodiments, the variant capsid polypeptide comprises a VP1, VP2, VP3, or any combination thereof, that each has 1 to 5 mutations as compared to a polypeptide of any one of SEQ ID NO:12 to SEQ ID NO:73, and optionally comprises at least one of, e.g., all of, the mutation differences of such SEQ ID NO:12 to SEQ ID NO:73.

In aspects, provided herein are nucleic acid molecules encoding a variant capsid polypeptide as provided herein. In aspects, the nucleic acid molecule comprises sequence encoding a variant capsid polypeptide (e.g., a VP1, VP2 or VP3 capsid polypeptide) of any one of SEQ ID NO:12 to SEQ ID NO:73, or fragment thereof. In aspects, the nucleic acid molecule comprises any one of SEQ ID NO:74 to SEQ ID NO:135, or fragment thereof (e.g., a VP1-encoding, VP2-encoding or VP3-encoding fragment thereof).

In some embodiments, the nucleic acid molecule encodes a variant capsid polypeptide that is at least 70%, 75%, 80%, 85%, 90%, 91%, 92%, 93%, 94%, 95%, 96%, 97%, 98%, 99%, or 100% identical to a variant capsid polypeptide as provided herein.

In some embodiments, the nucleic acid molecule or the nucleic acid molecule encoding the reference polypeptide for purposes of % identity, comprises a nucleotide sequence of SEQ ID NO:74 to SEQ ID NO:135, respectively.

In some embodiments, the nucleic acid molecule or the nucleic acid molecule encoding the reference polypeptide for purposes of % identity, comprises a nucleotide sequence that encodes a sequence of a variant capsid polypeptide, e.g., as described herein, e.g., encodes any one of SEQ ID NO:12 to SEQ ID NO:73.

In some embodiments, the variant capsid polypeptide, or the reference polypeptide for purposes of % identity, comprises a sequence of any one of SEQ ID NO:12 to SEQ ID NO:73, that is encoded by a nucleotide sequence of SEQ ID NO:74 to SEQ ID NO:135, respectively.

In some embodiments, the variant capsid polypeptide comprises a sequence that includes all of the mutation differences associated with any one of SEQ ID NO:12 to SEQ ID NO:73, e.g., relative to SEQ ID NO:1.

In some embodiments, the variant capsid polypeptide is a VP1 capsid polypeptide. In some embodiments, the variant capsid polypeptide is a VP2 capsid polypeptide. In some embodiments, the variant capsid polypeptide is a VP3 capsid polypeptide. With respect to reference sequence SEQ ID NO:1, a VP1 capsid polypeptide comprises amino acids 1-736 of SEQ ID NO:1. With respect to reference sequence SEQ ID NO:1, a VP2 capsid polypeptide comprises amino acids 138-736 of SEQ ID NO:1. With respect to reference sequence SEQ ID NO:1, a VP3 capsid polypeptide comprises amino acids 203-736 of SEQ ID NO:1.

With respect to variant capsid polypeptide sequence any one of SEQ ID NO:12 to SEQ ID NO:73, a VP1 capsid polypeptide comprises all of the amnio acids of any one of SEQ ID NO:12 to SEQ ID NO:73. With respect to a variant capsid polypeptide of any one of SEQ ID NO:12 to SEQ ID NO:73, a VP2 capsid polypeptide comprises, e.g., consists of, a sequence that begins with the threonine corresponding to the threonine at position 138 of SEQ ID NO:1, and continuing to the C-terminus of any one of SEQ ID NO:12 to SEQ ID NO:73. With respect to a sequence of any one of SEQ ID NO: 12 to SEQ ID NO:73, a VP3 capsid polypeptide comprises, e.g., consists of, a sequence that begins with the methionine corresponding to methionine at position 203 of SEQ ID NO:1 and continuing to the C-terminus of any one of SEQ ID NO:12 to SEQ ID NO:73.

Exemplary sequences of variant capsid polypeptides are provided in SEQ ID NO:12 to SEQ ID NO:73 and exemplary nucleic acid molecules encoding the same are provided in SEQ ID NO:74 to SEQ ID NO:135, respectively.

In some embodiments, the nucleic acid molecule encodes a variant capsid polypeptide that has at least 70, 75, 80, 85, 90, 91, 92, 93, 94, 95, 96, 97, 98, 99%, or 100% identity to a VP1, VP2, or VP3 sequence of any one of SEQ ID NO:12 to SEQ ID NO:73.

In some embodiments, the nucleic acid molecule encodes a variant capsid polypeptide that has at least 70, 75, 80, 85, 90, 91, 92, 93, 94, 95, 96, 97, 98, 99%, or 100% identity (not including targeting peptide insertions) to a VP1, VP2, or VP3 sequence of any one of SEQ ID NO:12 to SEQ ID NO:73.

### 5.2.1. Variant Capsid Polypeptides (Corresponding Positions)

The mutations to capsid polypeptide sequences described herein are described in relation to a position and/or amino acid at a position within a reference sequence, e.g., SEQ ID NO:1. Thus, in some embodiments, the capsid polypeptides described herein are variant capsid polypeptides of the reference sequence, e.g., SEQ ID NO:1, e.g., include capsid polypeptides comprising at least 80%, at least 85%, at least 90%, at least 91%, at least 92%, at least 93%, at least 94%, at least 95%, at least 96%, at least 97%, at least 98%, or at least 99% identity to the reference capsid polypeptide sequence (e.g., reference capsid polypeptide VP1, VP2 and/or VP3 sequence), e.g., SEQ ID NO:1 (or VP2 or VP3 sequence comprised therein) and include one or more mutations described herein.

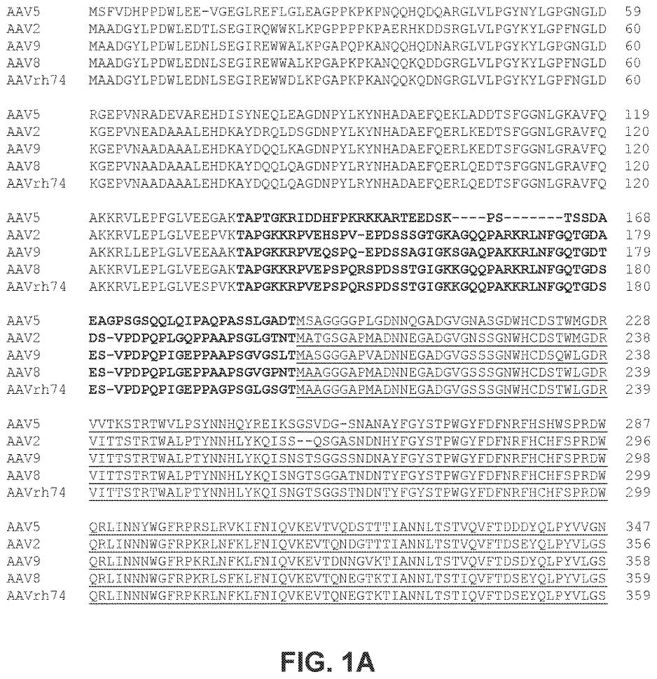

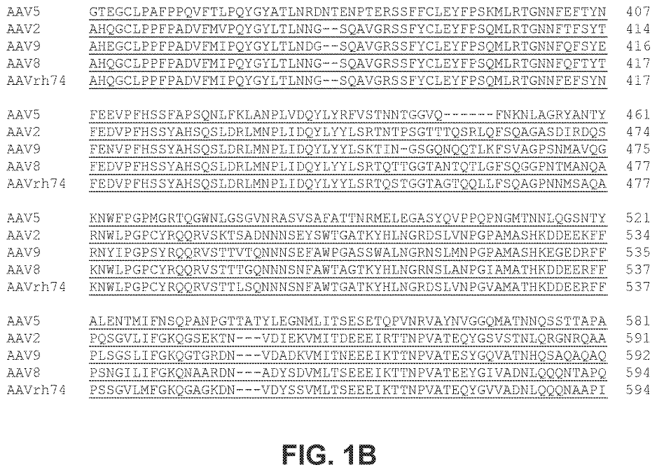

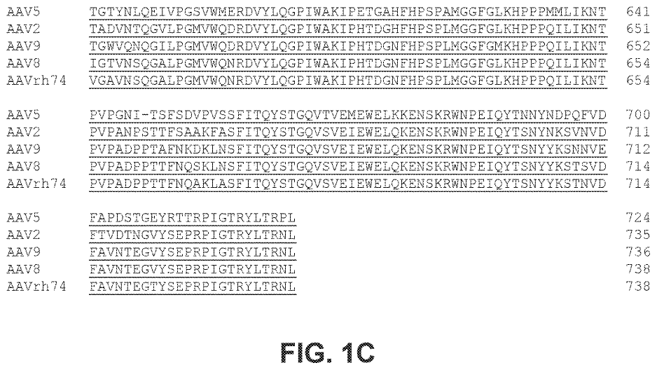

It will be understood by the skilled artisan, and without being bound by theory, that each amino acid position within a reference sequence corresponds to a position within the sequence of other reference capsid polypeptides such as capsid polypeptides derived from dependoparvoviruses with different serotypes. Such corresponding positions are identified using sequence alignment tools known in the art. A particularly preferred sequence alignment tool is EMBOSS Needle Pairwise Sequence Alignment software tool based on the Needleman and Wunsch algorithm (Needleman & Wunsch, 1970, J. Mol. Biol. 48(3):443-53) (available at www.ebi.ac.uk/Tools/psa/emboss_needle/). An alignment of exemplary reference capsid polypeptides is shown in FIGS. 1A-1C. Thus, in some embodiments, the variant capsid polypeptides of the invention include variants of reference capsid polypeptides that include one or more mutations described herein in such reference capsid polypeptides at positions corresponding to the position of the mutation described herein in relation to a different reference capsid polypeptide. Thus, for example, a mutation described as XnnnY relative to SEQ ID NO:1 (where X is the amino acid present at position nnn in SEQ ID NO:1 and Y is the amino acid mutation at that position, e.g., described herein), the disclosure provides variant capsid polypeptides comprising at least 70%, at least 75%, at least 80%, at least 85%, at least 90%, at least 91%, at least 92%, at least 93%, at least 94%, at least 95%, at least 96%, at least 97%, at least 98%, or at least 99% identity to a reference capsid polypeptide sequence (e.g., reference capsid polypeptide VP1, VP2 and/or VP3 sequence) other than SEQ ID NO:1 (or VP2 or VP3 sequence comprised therein) and further comprising the disclosed mutation at a position corresponding to position nnn of SEQ ID NO:1 (e.g., comprising Y at the position in the new variant capsid polypeptide sequence that corresponds to position nnn of SEQ ID NO:1). As described above, such corresponding position is determined using a sequence alignment tool, such as, for example, the clustal omega tool described above. Examples of corresponding amino acid positions of exemplary known AAV serotypes are provided in FIG. 1A-1C. In some embodiments, the variant is a variant of the AAV9 capsid polypeptide, which can be referred to as a “AAV9 variant capsid polypeptide” or “variant AAV9 capsid polypeptide.”

Thus, in embodiments, the disclosure provides variant capsid polypeptide sequences that are variants of a reference sequence other than SEQ ID NO:1, e.g., a reference sequence other than SEQ ID NO:1 as described herein, which include one or more mutation corresponding to the mutations described herein. In embodiments, such variants include mutations corresponding to all of the mutations associated with any one of SEQ ID NO:12 to SEQ ID NO:73.

The variant capsid polypeptides described herein are optionally variants of reference capsids serotypes known in the art. Non-limiting examples of such reference AAV serotypes include AAV1, AAVrh10, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.eB, AAVPHP.S/G2A12, AAVG2A15/G2A3 (G2A3), AAVG2B4 (G2B4), AAVG2B5 (G215), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9, AAV9 K449R (or K449R AAV9), AAV9.16, AAV9.24, AAV9.45, AAbiodisV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74 (also referred to as AAVrh74), AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LKO1, AAV-LKO2, AAV-LK03, AAV-LKO4, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9, 7m8, Spark100, AAVMYO and variants thereof.

In some embodiments, the reference AAV capsid sequence comprises an AAV2 sequence. In some embodiments, the reference AAV capsid sequence comprises an AAV5 sequence. In some embodiments, the reference AAV capsid sequence comprises an AAV8 sequence. In some embodiments, the reference AAV capsid sequence comprises an AAV9 sequence. In some embodiments, the reference AAV capsid sequence comprises an AAVrh74 sequence. While not wishing to be bound by theory, it is understood that a reference AAV capsid sequence comprises a VP1 region In certain embodiments, a reference AAV capsid sequence comprises a VP1, VP2 and/or VP3 region, or any combination thereof. A reference VP1 sequence may be considered synonymous with a reference AAV capsid sequence.

The wild-type reference sequence of AAV9, SEQ ID NO:1 is as follows:

(SEQ ID NO: 1)
MAADGYLPDWLEDNLSEGIREWWALKPGAPQPKANQQHQDNARGLVLP
GYKYLGPGNGLDKGEPVNAADAAALEHDKAYDQQLKAGDNPYLKYNHA
DAEFQERLKEDTSFGGNLGRAVFQAKKRLLEPLGLVEEAAKTAPGKKR
PVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVI
TTSTRTWALPTYNNHLYKQISNSTSGGSSNDNAYFGYSTPWGYFDFNR
FHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLND
GSQAVGRSSFYCLEYFPSQMLRTGNNFQFSYEFENVPFHSSYAHSQSL
DRLMNPLIDQYLYYLSKTINGSGQNQQTLKFSVAGPSNMAVQGRNYIP
GPSYRQQRVSTTVTQNNNSEFAWPGASSWALNGRNSLMNPGPAMASHK
EGEDRFFPLSGSLIFGKQGTGRDNVDADKVMITNEEEIKTTNPVATES
YGQVATNHQSAQAQAQTGWVQNQGILPGMVWQDRDVYLQGPIWAKIPH
TDGNFHPSPLMGGFGMKHPPPQILIKNTPVPADPPTAFNKDKLNSFIT
QYSTGQVSVEIEWELQKENSKRWNPEIQYTSNYYKSNNVEFAVNTEGV
YSEPRPIGTRYLTRNL

Unless otherwise noted, SEQ ID NO:1 is the reference sequence. In the sequence above, the sequence found in VP1, VP2 and VP3 is underlined (e.g., a VP3 capsid polypeptide includes, e.g., consists of, amino acids corresponding to amino acids 203-736 of SEQ ID NO:1), the sequence found in both VP1 and VP2 is in bold (e.g., a VP2 capsid polypeptide includes, e.g., consists of, the sequence corresponding to amino acids 138-736 of SEQ ID NO:1) and the sequence that is not underlined or bold is found only in VP1 (e.g., a VP1 capsid polypeptide includes, e.g., consists of, amino acids corresponding to amino acids 1-736 of SEQ ID NO:1).

The wild-type reference sequence of SEQ ID NO:1 can be encoded by a reference nucleic acid molecule sequence of SEQ ID NO:2.

An exemplary reference sequence of wild-type AAV2, SEQ ID NO:3 (wild-type AAV2) is as follows:

(SEQ ID NO: 3)
MAADGYLPDWLEDTLSEGIRQWWKLKPGPPPPKPAERHKDDSRGLVLP
GYKYLGPFNGLDKGEPVNEADAAALEHDKAYDRQLDSGDNPYLKYNHA
DAEFQERLKEDTSFGGNLGRAVFQAKKRVLEPLGLVEEPVKTAPGKKR
PVEHSPVEPDSSSGTGKAGQQPARKRLNFGQTGDADSVPDPQPLGQPP
AAPSGLGTNTMATGSGAPMADNNEGADGVGNSSGNWHCDSTWMGDRVI
TTSTRTWALPTYNNHLYKQISSQSGASNDNHYFGYSTPWGYFDFNRFH
CHFSPRDWQRLINNNWGFRPKRLNFKLFNIQVKEVTQNDGTTTIANNL
TSTVQVFTDSEYQLPYVLGSAHQGCLPPFPADVFMVPQYGYLTLNNGS
QAVGRSSFYCLEYFPSQMLRTGNNFTFSYTFEDVPFHSSYAHSQSLDR
LMNPLIDQYLYYLSRTNTPSGTTTQSRLQFSQAGASDIRDQSRNWLPG
PCYRQQRVSKTSADNNNSEYSWTGATKYHLNGRDSLVNPGPAMASHKD
DEEKFFPQSGVLIFGKQGSEKTNVDIEKVMITDEEEIRTTNPVATEQY
GSVSTNLQRGNRQAATADVNTQGVLPGMVWQDRDVYLQGPIWAKIPHT
DGHFHPSPLMGGFGLKHPPPQILIKNTPVPANPSTTFSAAKFASFITQ
YSTGQVSVEIEWELQKENSKRWNPEIQYTSNYNKSVNVDFTVDTNGVY
SEPRPIGTRYLTRNL.

In the sequence above, the sequence found in VP1, VP2 and VP3 is underlined (e.g., a VP3 capsid polypeptide includes, e.g., consists of, amino acids corresponding to amino acids 203-735 of SEQ ID NO:3), the sequence found in both VP1 and VP2 is in bold (e.g., a VP2 capsid polypeptide includes, e.g., consists of, the sequence corresponding to amino acids 138-735 of SEQ ID NO:3) and the sequence that is not underlined or bold is found only in VP1 (e.g., a VP1 capsid polypeptide includes, e.g., consists of, amino acids corresponding to amino acids 1-735 of SEQ ID NO:3).

An example nucleic acid sequence encoding SEQ ID NO:3 is SEQ ID NO:4.

An exemplary reference sequence of wild type AAV5, SEQ ID NO:5 (wild-type AAV5), is as follows:

(SEQ ID NO: 5)
MSFVDHPPDWLEEVGEGLREFLGLEAGPPKPKPNQQHQDQARGLVLPG
YNYLGPGNGLDRGEPVNRADEVAREHDISYNEQLEAGDNPYLKYNHAD
AEFQEKLADDTSFGGNLGKAVFQAKKRVLEPFGLVEEGAKTAPTGKRI
DDHFPKRKKARTEEDSKPSTSSDAEAGPSGSQQLQIPAQPASSLGADT
MSAGGGGPLGDNNQGADGVGNASGDWHCDSTWMGDRVVTKSTRTWVLP
SYNNHQYREIKSGSVDGSNANAYFGYSTPWGYFDFNRFHSHWSPRDWQ
RLINNYWGFRPRSLRVKIFNIQVKEVTVQDSTTTIANNLTSTVQVFTD
DDYQLPYVVGNGTEGCLPAFPPQVFTLPQYGYATLNRDNTENPTERSS
FFCLEYFPSKMLRTGNNFEFTYNFEEVPFHSSFAPSQNLFKLANPLVD
QYLYRFVSTNNTGGVQFNKNLAGRYANTYKNWFPGPMGRTQGWNLGSG
VNRASVSAFATTNRMELEGASYQVPPQPNGMTNNLQGSNTYALENTMI
FNSQPANPGTTATYLEGNMLITSESETQPVNRVAYNVGGQMATNNQSS
TTAPATGTYNLQEIVPGSVWMERDVYLQGPIWAKIPETGAHFHPSPAM
GGFGLKHPPPMMLIKNTPVPGNITSFSDVPVSSFITQYSTGQVTVEME
WELKKENSKRWNPEIQYTNNYNDPQFVDFAPDSTGEYRTTRPIGTRYL
TRPL

In the sequence above, the sequence found in VP1, VP2 and VP3 is underlined (e.g., a VP3 capsid polypeptide includes, e.g., consists of, amino acids corresponding to amino acids 193-724 of SEQ ID NO:5), the sequence found in both VP1 and VP2 is in bold (e.g., a VP2 capsid polypeptide includes, e.g., consists of, the sequence corresponding to amino acids 137-724 of SEQ ID NO:5) and the sequence that is not underlined or bold is found only in VP1 (e.g., a VP1 capsid polypeptide includes, e.g., consists of, amino acids corresponding to amino acids 1-724 of SEQ ID NO:5).

An example nucleic acid sequence encoding SEQ ID NO:5 is SEQ ID NO:6.

An exemplary reference sequence of wild-type AAV8, SEQ ID NO:7 (wild-type AAV8), is as follows:

(SEQ ID NO: 7)
MAADGYLPDWLEDNLSEGIREWWALKPGAPKPKANQQKQDDGRGLVLP
GYKYLGPFNGLDKGEPVNAADAAALEHDKAYDQQLQAGDNPYLRYNHA
DAEFQERLQEDTSFGGNLGRAVFQAKKRVLEPLGLVEEGAKTAPGKKR
PVEPSPQRSPDSSTGIGKKGQQPARKRLNFGQTGDSESVPDPQPLGEP
PAAPSGVGPNTMAAGGGAPMADNNEGADGVGSSSGNWHCDSTWLGDRV
ITTSTRTWALPTYNNHLYKQISNGTSGGATNDNTYFGYSTPWGYFDFN
RFHCHFSPRDWQRLINNNWGFRPKRLSFKLFNIQVKEVTQNEGTKTIA
NNLTSTIQVFTDSEYQLPYVLGSAHQGCLPPFPADVFMIPQYGYLTLN
NGSQAVGRSSFYCLEYFPSQMLRTGNNFQFTYTFEDVPFHSSYAHSQS
LDRLMNPLIDQYLYYLSRTQTTGGTANTQTLGFSQGGPNTMANQAKNW
LPGPCYRQQRVSTTTGQNNNSNFAWTAGTKYHLNGRNSLANPGIAMAT
HKDDEERFFPSNGILIFGKQNAARDNADYSDVMLTSEEEIKTTNPVAT
EEYGIVADNLQQQNTAPQIGTVNSQGALPGMVWQNRDVYLQGPIWAKI
PHTDGNFHPSPLMGGFGLKHPPPQILIKNTPVPADPPTTFNQSKLNSF
ITQYSTGQVSVEIEWELQKENSKRWNPEIQYTSNYYKSTSVDFAVNTE
GVYSEPRPIGTRYLTRNL

In the sequence above, the sequence found in VP1, VP2 and VP3 is underlined (e.g., a VP3 capsid polypeptide includes, e.g., consists of, amino acids corresponding to amino acids 204-738 of SEQ ID NO:7), the sequence found in both VP1 and VP2 is in bold (e.g., a VP2 capsid polypeptide includes, e.g., consists of, the sequence corresponding to amino acids 138-738 of SEQ ID NO:7) and the sequence that is not underlined or bold is found only in VP1 (e.g., a VP1 capsid polypeptide includes, e.g., consists of, amino acids corresponding to amino acids 1-738 of SEQ ID NO:7).

An example nucleic acid sequence encoding SEQ ID NO:7 is SEQ ID NO:8

An exemplary reference sequence of wild-type AAVrh74, SEQ ID NO:9 (wild-type AAVrh74), is as follows:

(SEQ ID NO: 9)
MAADGYLPDWLEDNLSEGIREWWDLKPGAPKPKANQQKQDNGRGLVLP
GYKYLGPFNGLDKGEPVNAADAAALEHDKAYDQQLQAGDNPYLRYNHA
DAEFQERLQEDTSFGGNLGRAVFQAKKRVLEPLGLVESPVKTAPGKKR
PVEPSPQRSPDSSTGIGKKGQQPAKKRLNFGQTGDSESVPDPQPIGEP
PAGPSGLGSGTMAAGGGAPMADNNEGADGVGSSSGNWHCDSTWLGDRV
ITTSTRTWALPTYNNHLYKQISNGTSGGSTNDNTYFGYSTPWGYFDFN
RFHCHFSPRDWQRLINNNWGFRPKRLNFKLFNIQVKEVTQNEGTKTIA
NNLTSTIQVFTDSEYQLPYVLGSAHQGCLPPFPADVFMIPQYGYLTLN
NGSQAVGRSSFYCLEYFPSQMLRTGNNFEFSYNFEDVPFHSSYAHSQS
LDRLMNPLIDQYLYYLSRTQSTGGTAGTQQLLFSQAGPNNMSAQAKNW
LPGPCYRQQRVSTTLSQNNNSNFAWTGATKYHLNGRDSLVNPGVAMAT
HKDDEERFFPSSGVLMFGKQGAGKDNVDYSSVMLTSEEEIKTTNPVAT
EQYGVVADNLQQQNAAPIVGAVNSQGALPGMVWQNRDVYLQGPIWAKI
PHTDGNFHPSPLMGGFGLKHPPPQILIKNTPVPADPPTTFNQAKLASF
ITQYSTGQVSVEIEWELQKENSKRWNPEIQYTSNYYKSTNVDFAVNTE
GTYSEPRPIGTRYLTRNL

An alternative exemplary reference sequence of SEQ ID NO:11 (alternate wild-type AAVrh74) is as follows:

(SEQ ID NO: 11)
MAADGYLPDWLEDNLSEGIREWWDLKPGAPKPKANQQKQDNGRGLVLP
GYKYLGPFNGLDKGEPVNAADAAALEHDKAYDQQLQAGDNPYLRYNHA
DAEFQERLQEDTSFGGNLGRAVFQAKKRVLEPLGLVESPVKTAPGKKR
PVEPSPQRSPDSSTGIGKKGQQPAKKRLNFGQTGDSESVPDPQPIGEP
PAGPSGLGSGTMAAGGGAPMADNNEGADGVGSSSGNWHCDSTWLGDRV
ITTSTRTWALPTYNNHLYKQISNGTSGGSTNDNTYFGYSTPWGYFDFN
RFHCHFSPRDWQRLINNNWGFRPKRLNFKLFNIQVKEVTQNEGTKTIA
NNLTSTIQVFTDSEYQLPYVLGSAHQGCLPPFPADVFMIPQYGYLTLN
NGSQAVGRSSFYCLEYFPSQMLRTGNNFEFSYNFEDVPFHSSYAHSQS
LDRLMNPLIDQYLYYLSRTQSTGGTAGTQQLLFSQAGPNNMSAQAKNW
LPGPCYRQQRVSTTLSQNNNSNFAWTGATKYHLNGRDSLVNPGVAMAT
HKDDEERFFPSSGVLMFGKQGAGKDNVDYSSVMLTSEEEIKTTNPVAT
EQYGVVADNLQQQNAAPIVGAVNSQGALPGMVWQNRDVYLQGPIWAKI
PHTDGNFHPSPLMGGFGLKHPPPQILIKNTPVPADPPTTFTKAKLASF
ITQYSTGQVSVEIEWELQKENSKRWNPEIQYTSNYYKSTNVDFAVNTE
GTYSEPRPIGTRYLTRNL

In the sequences above (SEQ ID NO:9 or SEQ ID NO:11), the sequence found in VP1, VP2 and VP3 is underlined (e.g., a VP3 capsid polypeptide includes, e.g., consists of, amino acids corresponding to amino acids 204-738 of SEQ ID NO:9), the sequence found in both VP1 and VP2 is in bold (e.g., a VP2 capsid polypeptide includes, e.g., consists of, the sequence corresponding to amino acids 137-738 of SEQ ID NO:9) and the sequence that is not underlined or bold is found only in VP1 (e.g., a VP1 capsid polypeptide includes, e.g., consists of, amino acids corresponding to amino acids 1-738 of SEQ ID NO:9).

An example nucleic acid sequence encoding SEQ ID NO:9 is SEQ ID NO:10.

The present disclosure refers to structural capsid proteins (including VP1, VP2 and VP3) which are encoded by capsid (Cap) genes. These capsid proteins form an outer protein structural shell (i.e., capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins. Where the Met/AA-clipping is incomplete, a mixture of one or more (one, two or three) VP capsid proteins comprising the viral capsid can be produced, some of which include a Met1/AA1 amino acid (Met+/AA+) and some of which lack a Met1/AA1 amino acid as a result of Met/AA-clipping (Met-/AA-). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods 2017 Oct. 28(5):255-267; Hwang, et al. N-Terminal Acetylation of Cellular Proteins Creates Specific Degradation Signals. Science. 2010 February 19.327(5968): 973-977; the contents of which are each incorporated herein by reference in its entirety. According to the present disclosure, references to capsid polypeptides is not limited to either clipped (Met−/AA−) or unclipped (Met+/AA+) and, in context, also refer to independent capsid polypeptides, viral capsids comprised of a mixture of capsid proteins, and/or polynucleotide sequences (or fragments thereof) which encode, describe, produce or result in capsid polypeptides of the present disclosure. A direct reference to a “capsid polypeptide” (such as VP1, VP2 or VP3) also comprise VP capsid proteins which include a Met1/AA1 amino acid (Met+/AA+) as well as corresponding VP capsid polypeptide which lack the Met1/AA1 amino acid as a result of Met/AA-clipping (Met−/AA−). Further according to the present disclosure, a reference to a specific SEQ ID NO:(whether a protein or nucleic acid) which comprises or encodes, respectively, one or more capsid polypeptides which include a Met1/AA1 amino acid (Met+/AA+) should be understood to teach the VP capsid polypeptides which lack the Met1/AA1 amino acid as upon review of the sequence, it is readily apparent any sequence which merely lacks the first listed amino acid (whether or not Met1/AA1). As a non-limiting example, reference to a VP1 polypeptide sequence which is 736 amino acids in length and which includes a “Met1” amino acid (Met+) encoded by the AUG/ATG start codon is also understood to teach a VP1 polypeptide sequence which is 735 amino acids in length and which does not include the “Met1” amino acid (Met−) of the 736 amino acid Met+sequence. As a second non-limiting example, reference to a VP1 polypeptide sequence which is 736 amino acids in length and which includes an “AA1” amino acid (AA1+) encoded by any NNN initiator codon can also be understood to teach a VP1 polypeptide sequence which is 735 amino acids in length and which does not include the “AA1” amino acid (AA1−) of the 736 amino acid AA1+sequence. References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1−), and combinations thereof (Met+/AA1+ and Met−/AA1−). As a non-limiting example, an AAV capsid serotype can include VP1 (Met+/AA1+), VP1 (Met−/AA1−), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

In some embodiments, the reference AAV capsid sequence comprises an amino acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the those described above.

In some embodiments, the reference AAV capsid sequence is encoded by a nucleotide sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of those described above. In certain embodiments, the reference sequence is not an AAV capsid sequence and is instead a different vector (e.g., lentivirus, plasmid, etc.).

In some embodiments, a nucleic acid of the disclosure (e.g., encoding an AAV9 variant capsid protein) comprises conventional control elements or sequences which are operably linked to the nucleic acid molecule in a manner which permits transcription, translation and/or expression in a cell transfected with the nucleic acid (e.g., a plasmid vector comprising said nucleic acid) or infected with a virus comprising said nucleic acid. As used herein, “operably linked” sequences include both expression control sequences that are contiguous with the gene of interest and expression control sequences that act in trans or at a distance to control the gene of interest.

Expression control sequences include efficient RNA processing signals such as splicing and polyadenylation (polyA) signals; appropriate transcription initiation, termination, promoter and enhancer sequences; sequences that stabilize cytoplasmic mRNA; sequences that enhance protein stability; sequences that enhance translation efficiency (e.g., Kozak consensus sequence); and in some embodiments, sequences that enhance secretion of the encoded transgene product. Expression control sequences, including promoters which are native, constitutive, inducible and/or tissue-specific, are known in the art and can be utilized with the compositions and methods disclosed herein.

In some embodiments, the native promoter for the transgene is used. Without wishing to be bound by theory, the native promoter can mimic native expression of the transgene, or provide temporal, developmental, or tissue-specific expression, or expression in response to specific transcriptional stimuli. In some embodiments, the transgene is operably linked to other native expression control elements, such as enhancer elements, polyadenylation sites or Kozak consensus sequences, e.g., to mimic the native expression.

In some embodiments, the transgene is operably linked to a tissue-specific promoter, e.g., a promoter active specifically in one or more CNS cell types. In some embodiments, the transgene is operably linked to a promoter active in skeletal muscle. The promoter active in skeletal muscle can be specific to skeletal muscle, or expressed more broadly in other muscle types.

In some embodiments, a vector, e.g., a plasmid, carrying a transgene includes a selectable marker or a reporter gene. Such selectable reporters or marker genes can be used to signal the presence of the vector, e.g., plasmid, in bacterial cells. Other components of the vector, e.g., plasmid, include an origin of replication. Selection of these and other promoters and vector elements are conventional, and many such sequences are available (see, e.g., Sambrook et al, and references cited therein).

In some embodiments, the viral particle comprising a variant capsid polypeptide, e.g., a variant capsid polypeptide described herein, exhibits increased CNS transduction as compared to a viral particle with the wild-type capsid polypeptide (SEQ ID NO:1).

In some embodiments, the viral particle comprising a variant capsid polypeptide, e.g., a variant capsid polypeptide described herein, exhibits increased skeletal muscle transduction as compared to a viral particle with the wild-type capsid polypeptide (SEQ ID NO:1).

In some embodiments, the capsid polypeptide is an isolated or purified polypeptide (e.g., isolated or purified from a cell, other biological component, or contaminant). In some embodiments, the variant polypeptide is present in a dependoparvovirus particle, e.g., described herein. In some embodiments, the variant capsid polypeptide is present in a cell, cell-free system, or translation system, e.g., described herein.

In some embodiments, the capsid polypeptide is present in a dependoparvovirus B (e.g., AAV9) particle. In some embodiments, the capsid particle has increased CNS transduction. In some embodiments, the capsid particle has increased skeletal muscle transduction.

In some embodiments, a dependoparvovirus particle comprises an amino acid sequence that has at least 80, 85, 90, 91, 92, 93, 94, 95, 96, 97, 98, 99, or 100% identity to the amino acid sequences provided for herein (e.g., any one of SEQ ID NO:12 to SEQ ID NO:73). In some embodiments, the variant capsid polypeptide comprises an amino acid sequence that differs by no more than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 amino acids from the amino acid sequence of a variant capsid polypeptide provided for herein.

In some embodiments, the additional alteration improves a production characteristic of a dependoparvovirus particle or method of making the same. In some embodiments, the additional alteration improves or alters another characteristic of a dependoparvovirus particle, e.g., tropism.

### 5.2.1. Targeting Peptides

The capsid polypeptides of the disclosure can include (but do not necessarily include) a targeting peptide to alter the tropism of the capsid polypeptides, for example to enhance targeting to the CNS or skeletal muscle. Thus, in some embodiments, a capsid polypeptide of the disclosure includes a targeting peptide. In other embodiments, a capsid polypeptide of the disclosure does not include a targeting peptide.

### 5.2.1.1. CNS Targeting Peptides

Various targeting peptides for enhancing CNS tropism, and which can be included in capsid polypeptides of the disclosure, are described in the art, for example in WO 2017/197355, WO 2019/006182, WO 2019/060454, WO 2012/145601, WO 2018/022905, WO 2021/243085, WO 2019/076856, WO2015/038958, WO 2015/191508, WO 2020/068990, WO 2020/210655, WO 2020/198737, WO 2020/028751, WO 2019/028306, WO 2017/100671 A1, WO 2020/028751 A2, WO 2020/072683 A1, WO 2020/160337 A1, WO 2020/223280 A1, WO 2021/025995 A1, WO 2021/202651 A1, WO 2021/230987 A1, WO 2022/235702 A1, WO 2020/014471, WO 2018/189244, WO 2019/141765, WO 2019/207132, WO 2019/210267, WO 2018/156654, WO 2010/093784, WO 2015/048534, WO 2017/058892, WO 2019/169132, WO 2021/108468, WO 2021/102234, WO 2022/173847, WO 2021/077000, WO 2020/160337, WO 2021/050974, WO 2021/222831, WO 2022/020616, WO 2020/193799, WO 2021/072197, WO 2022/126188, WO 2022/126189, WO 2021/165544, WO 2021/084133, WO 2022/040527, WO 2022/221400, WO 2022/221404, WO 2022/221420, WO 2021/216456, WO 2021/009684, WO 2021/242909, WO 2019/158619, WO 2021/226267, WO 2023/283962, WO 2021/219762, WO 2022/226374, WO 2022/226375, WO 2022/229703, and WO 2022/229702, the contents of which are incorporated herein by reference in their entireties. Targeting peptides are typically 3 to 20 amino acids in length. In some embodiments, a targeting peptide is 3 to 12 amino acids in length. In other embodiments, a targeting peptide is 5 to 12 amino acids in length. In other embodiments, a targeting peptide is 5 to 10 amino acids in length. In other embodiments, a targeting peptide is 7 to 10 amino acids in length. In some embodiments, a targeting peptide is 7 amino acids in length. In other embodiments, a targeting peptide is 9 amino acids in length.

In some embodiments, the targeting peptide comprises at least 3, 4, 5, 6, 7, 8, or 9 consecutive amino acids from the amino acid sequence of PLNGAVHLY (SEQ ID NO:255). In some embodiments, the targeting peptide comprises the amino acid sequence PLNGAVHLY (SEQ ID NO:255). In some embodiments, the targeting peptide comprises at least 3, 4, 5, 6, or 7 consecutive amino acids from the amino acid sequence of IVMNSLK (SEQ ID NO:256). In some embodiments, the targeting peptide comprises the amino acid sequence IVMNSLK (SEQ ID NO:256). In some embodiments, the targeting peptide comprises at least 3, 4, 5, 6, or 7 consecutive amino acids from the amino acid sequence of RDSPKGW (SEQ ID NO:257). In some embodiments, the targeting peptide comprises the amino acid sequence RDSPKGW (SEQ ID NO:257). In some embodiments, the targeting peptide comprises at least 3, 4, 5, 6, or 7 consecutive amino acids from the amino acid sequence of YSTDVRM (SEQ ID NO:258). In some embodiments, the targeting peptide comprises the amino acid sequence YSTDVRM (SEQ ID NO:258). In some embodiments, the targeting peptide comprises at least 3, 4, 5, 6, or 7 consecutive amino acids from the amino acid sequence of RESPRGL (SEQ ID NO:259). In some embodiments, the targeting peptide comprises the amino acid sequence RESPRGL (SEQ ID NO:259). In some embodiments, the targeting peptide comprises 4, 5, 6, or 7 consecutive amino acids from GNNTRSV (SEQ ID NO:260), GNNTRDT (SEQ ID NO:261) or TNSTRPV (SEQ ID NO:262). In some embodiments, the targeting peptide comprises the amino acid sequence GNNTRSV (SEQ ID NO:260). In some embodiments, the targeting peptide comprises the amino acid sequence GNNTRDT (SEQ ID NO:261). In some embodiments, the targeting peptide comprises the amino acid sequence TNSTRPV (SEQ ID NO:262).

In some embodiments, the CNS-targeting peptide is present in, e.g., inserted into, loop VIII of the capsid polypeptide. In some embodiments, the targeting peptide is inserted at any amino acid position corresponding to positions 586-592, inclusive, of the wild-type capsid polypeptide (SEQ ID NOA:1). For example, the targeting peptide can be inserted between amino acids 588-589 (positions corresponding to the wild-type capsid polypeptide (SEQ ID NO:1)). In some embodiments, the targeting peptide is present, e.g., inserted, immediately subsequent to the position corresponding to 586, 588, or 589 of the wild-type capsid polypeptide (SEQ ID NO:1). In some embodiments, the capsid polypeptide further comprises a deletion at the position corresponding to 587 and/or a deletion at the position corresponding to 588 of the wild-type capsid polypeptide (SEQ ID NO:1).

### 5.2.1.2. Muscle Targeting Peptides

Various targeting peptides for enhancing skeletal muscle tropism, and which can be included in capsid polypeptides of the disclosure, are described in the art, for example in WO 2020/206189A1, WO 2022/226374 A1, WO 2022/053630 A1, and WO 2019/207132 A1.

In some embodiments, the muscle targeting peptide comprises at least 3, 4, 5, 6, or 7 consecutive amino acids from the amino acid sequence ASSLNIA (SEQ ID NO:263).

In some aspects, the targeting peptide targets the insulin receptor (INSR). In some embodiments, the peptide targeting the INSR comprises an amino acid sequence having at least 80%, 87%, 91%, 94%, 97%, or 100% sequence identity to SLEEEWAQVECEVYGRGCPSGSLDESFYDWFERQL (SEQ ID NO:264), or which has at least 25, 26, 27, 28, 29, 30, 31, 32, 33, 34 or 35 consecutive amino acids from the amino acid sequence SLEEEWAQVECEVYGRGCPSGSLDESFYDWFERQL (SEQ ID NO:264).

In some aspects, the targeting peptide targets muscle-specific kinase (MUSK). In some embodiments, the inserted MUSK-targeting peptide is from the acetylcholinesterase collagenic tail peptide (CoIQ), e.g., a C-terminal portion of CoIQ (CoIQ CTD). In some embodiments, the CoIQ CTD peptide comprises an amino acid sequence having at least 80%, 85%, 90%, 95%, 96%, 97%, 98%, 99%, or 100% sequence identity to TPFYPVGYTVKQPGTCGDGVLQPGEECDDGNPDVSDGCIDCHRAYCGDGYRHQGVEDCDGSDFGYLT CETYLPGSYGDLRCTQYCSIDSTPCRYFT (SEQ ID NO:265), or which has at least 70, 80, 85, 90, 91, 92, 93, 94, 95, or 96 consecutive amino acids from the amino acid sequence

(SEQ ID NO: 265)
TPFYPVGYTVKQPGTCGDGVLQPGEECDDGNPDVSDGCIDCHRAYCGD
GYRHQGVEDCDGSDFGYLTCETYLPGSYGDLRCTQYCSIDSTPCRYFT.

In some aspects, the targeting peptide targets integrin, for example via an RGD-motif. In some embodiments, the RGD peptide comprises a subsequence Y or F amino acid to produce an RGDY (SEQ ID NO:266) or RGDF (SEQ ID NO:267) motif. In some embodiments, integrin targeting peptides have the motif RGDX₁X₂X₃X₄, with X₁ to X₄ each being any amino acid. In various aspects of the motif RGDX₁X₂X₃X₄, X₁, X₂, and X₃ are each independently selected from L, G, V, and A and/or X₄ is S, V, A, G, or L. In some embodiments, at least one of X₂ and X₃ is G. In some embodiments, the targeting peptide comprises the amino acid sequence RGDLGLS (SEQ ID NO: 269). In some embodiments, the targeting peptide comprises the amino acid sequence RGDLSTP (SEQ ID NO:270). In some embodiments, the targeting peptide comprises the amino acid sequence SNSRGDYNSL (SEQ ID NO:271). In some embodiments, the targeting peptide comprises the amino acid sequence ENRRGDFNNT (SEQ ID NO:272). In some embodiments, the targeting peptide comprises the amino acid sequence SRGDYNSL (SEQ ID NO:273). In some embodiments, the targeting peptide comprises the amino acid sequence RGDYNSL (SEQ ID NO-274). In some embodiments, the targeting peptide comprises the amino acid sequence RGDLST (SEQ ID NO: 275). In some embodiments, the targeting peptide comprises the amino acid sequence RGDYVGL (SEQ ID NO:276). In some embodiments, the targeting peptide comprises the amino acid sequence RGDAVGV (SEQ ID NO:277). The RGD peptides can be inserted in a linker, e.g., a flexible linker such as GGGS (SEQ ID NO:278), scaffold. Other suitable linker scaffolds can be found in WO 2022/226374 A1, pages 26-28 of which are incorporated herein by reference.

In some embodiments, the skeletal muscle-targeting peptide is present in, e.g., inserted into, loop VIII of the capsid polypeptide. In some embodiments, the targeting peptide is inserted at any amino acid position corresponding to positions 586-592, inclusive, of the wild-type capsid polypeptide (SEQ ID NO:1). For example, the targeting peptide can be inserted between amino acids 588-589 (positions corresponding to the wild-type capsid polypeptide (SEQ ID NO:1)). In some embodiments, the targeting peptide is present, e.g., inserted, immediately subsequent to the position corresponding to 586, 588, or 589 of the wild-type capsid polypeptide (SEQ ID NO:1). In some embodiments, the capsid polypeptide further comprises a deletion at the position corresponding to 587 and/or a deletion at the position corresponding to 588 of the wild-type capsid polypeptide (SEQ ID NO:1).

### 5.2.2. Nucleic Acids and Polypeptides

The disclosure is further directed, in part, to a nucleic acid comprising a sequence encoding a variant capsid polypeptide as provided for herein. In embodiments the nucleic acid encodes a VP1 variant capsid polypeptide, e.g., as described herein. In embodiments, the nucleic acid encodes a VP2 variant capsid polypeptide, e.g., as described herein. In embodiments, the nucleic acid encodes a VP3 variant capsid polypeptide, e.g., as described herein. In embodiments, the nucleic acid encodes a VP1, VP2 and VP3 variant capsid polypeptide, e.g., as described herein. In some embodiments, the variant capsid polypeptide comprises a sequence of any one of SEQ ID NO:12 to SEQ ID NO:73. In some embodiments, the nucleic acid comprises SEQ ID NO:74 to SEQ ID NO:135, respectively.

### 5.3. Dependoparvovirus Particles

The disclosure is also directed, in part, to a dependoparvovirus particle (e.g., a functional dependoparvovirus particle) comprising a nucleic acid or polypeptide described herein or produced by a method described herein.

Dependoparvovirus is a single-stranded DNA parvovirus that grows only in cells in which certain functions are provided, e.g., by a co-infecting helper virus. Several species of dependoparvovirus are known, including dependoparvovirus A and dependoparvovirus B, which include serotypes known in the art as adeno-associated viruses (AAV). At least thirteen serotypes of AAV that have been characterized. General information and reviews of AAV can be found in, for example, Carter, Handbook of Parvoviruses, Vol. 1, pp. 169-228 (1989), and Berns, Virology, pp. 1743-1764, Raven Press, (New York, 1990). AAV serotypes, and to a degree, dependoparvovirus species, are significantly interrelated structurally and functionally. (See, for example, Blacklowe, pp. 165-174 of Parvoviruses and Human Disease, J. R. Pattison, ed. (1988); and Rose, Comprehensive Virology 3:1-61 (1974)). For example, all AAV serotypes apparently exhibit very similar replication properties mediated by homologous rep genes; and all bear three related capsid proteins. In addition, heteroduplex analysis reveals extensive cross-hybridization between serotypes along the length of the genome, further suggesting interrelatedness. Dependoparvoviruses genomes also comprise self-annealing segments at the termini that correspond to “inverted terminal repeat sequences” (ITRs).

The genomic organization of naturally occurring dependoparvoviruses, e.g., AAV serotypes, is very similar. For example, the genome of AAV is a linear, single-stranded DNA molecule that is approximately 5,000 nucleotides (nt) in length or less. Inverted terminal repeats (ITRs) flank the unique coding nucleotide sequences for the non-structural replication (Rep) proteins and the structural capsid (Cap) proteins. Three different viral particle (VP) proteins form the capsid. The terminal approximately 145 nt of the genome are self-complementary and are organized so that an energetically stable intramolecular duplex forming a T-shaped hairpin may be formed. These hairpin structures function as an origin for viral DNA replication, serving as primers for the cellular DNA polymerase complex. The Rep genes encode the Rep proteins: Rep78, Rep68, Rep52, and Rep40. Rep78 and Rep68 are transcribed from the p5 promoter, and Rep 52 and Rep40 are transcribed from the p19 promoter. The cap genes encode the VP proteins, VP1, VP2, and VP3. The cap genes are transcribed from the p40 promoter.

In some embodiments, a dependoparvovirus particle of the disclosure comprises a nucleic acid comprising a variant capsid polypeptide provided for herein. In some embodiments, the particle comprises a polypeptide as provided for herein.

In some embodiments, the dependoparvovirus particle of the disclosure is an AAV9 particle. In some embodiments, the AAV9 particle comprises a variant capsid polypeptide as provided for herein or a nucleic acid molecule encoding the same.

In some embodiments the dependoparvovirus particle comprises a variant capsid comprising a variant capsid polypeptide described herein. In embodiments, the dependoparvovirus particle comprises variant capsid polypeptide described herein and a nucleic acid molecule. In embodiments, the dependoparvovirus particle comprises variant capsid polypeptide described herein and a nucleic acid molecule comprising one or more inverted terminal repeat sequences (ITRs), for example, ITRs derived from an AAV9 dependoparvovirus or an AAV2 dependoparvovirus, one or more regulatory elements (for example, a promoter), and a payload (e.g., as described herein, e.g., a heterologous transgene). In embodiments, at least one of the ITRs is modified. In embodiments, the nucleic acid molecule is single-stranded. In embodiments, the nucleic acid molecule is double stranded, for example, self-complementary.

### 5.4. Improved Biodistribution and Transduction Characteristics

The disclosure is directed, in part, to nucleic acids, polypeptides, cells, cell free systems, translation systems, viral particles, and methods associated with using and making the same to produce viral particles that have increased distribution to tissues and cells of the CNS and/or CNS transduction as compared to a viral particle comprising a reference sequence that does not otherwise comprise the mutations described herein (or mutations corresponding thereto), for example, as compared with a viral particle comprising a capsid polypeptide sequence of SEQ ID NO:1. In some embodiments, a use of a viral particle comprising the variant capsid polypeptides as described in Section 5.2 or any one of numbered embodiments 1 to 531 (e.g., a viral particle as described in Section 5.3, Section 5.4, or any one of numbered embodiments 552 to 736) leads to increased CNS biodistribution of the viral particle and/or increased transduction of a transgene virus particle in the cells of the CNS broadly or brain specifically, and, therefore, increased expression of the payload (transgene) in the CNS or brain. In some embodiments, use of a viral particle comprising the variant capsid polypeptides as described in Section 5.2 or any one of numbered embodiments 1 to 531 (e.g., a viral particle as described in Section 5.3 and Section 5.4 or any one of numbered embodiments 552 to 736) further leads to reduced (or non-increased) biodistribution of the viral particle and/or reduced (or non-increased) transduction of the transgene in one or more peripheral tissues, e.g., liver, spleen, dorsal root ganglia, or any combination of two or more of the foregoing peripheral tissue types. In some embodiments, use of a viral particle comprising the variant capsid polypeptides as described in Section 5.2 or any one of numbered embodiments 1 to 531 (e.g., a viral particle as described in Section 5.3 and Section 5.4 or any one of numbered embodiments 552 to 736) further leads to increased skeletal muscle biodistribution of the viral particle and/or increased transduction of a transgene virus particle in skeletal muscle cells, and, therefore, increased expression of the payload (transgene) in skeletal muscle.

In some embodiments, biodistribution and transduction (e.g., of the tissue types described in this section) are measured as described herein, for example as described in Section 7 (e.g., any of Examples 2, 4 and 5, for example by relative quantification (e.g., via qPCR) of transgene mRNA in one or more samples isolated from the relevant tissue type, e.g., CNS or skeletal muscle). In some embodiments, biodistribution and/or transduction of a virus particle having a variant capsid polypeptide can be measured using virus particles having a transgene operably linked to a promoter that is active in a target cell or tissue type of interest. In some embodiments, the promoter is a ubiquitous promoter. In other embodiments, the promoter is selective or specific to a target cell or tissue type (e.g., CNS and/or muscle (either broad muscle expression or skeletal muscle)), and, optionally, is less (or not) active in a cell or tissue type where transgene expression is not desired (e.g., liver, spleen, PNS, or any combination of two or all of the foregoing). A promoter that is selective for a first cell or tissue type over a second cell or tissue type is active in the first cell or tissue type and less active or silent in the second cell or tissue type In some embodiments, the promoter is a CNS-specific or CNS-selective promoter. In some embodiments, the promoter is a muscle-specific promoter-specific or muscle-selective promoter. In some embodiments, the promoter is active in both CNS and muscle tissue. In some embodiments, biodistribution and/or transduction of a virus particle having a variant capsid polypeptide can be measured using virus particles having a transgene operably linked to a ubiquitous promoter or a CNS-specific promoter. For example, biodistribution can be measured using virus particles having a transgene operably linked to CBh promoter or a hSYN promoter. In various embodiments, the transgene is a transgene encoding a capsid polypeptide or any other suitable heterologous transgene, for example a nucleic acid sequence encoding a synthetic, mammalian or human therapeutic protein or nucleic acid (e.g., mRNA or RNAi) or reporter gene such as, for example a nucleic acid encoding a GFP or mCherry reporter.

In embodiments, the virus particle, e.g., as described herein, e.g., comprising a variant capsid polypeptide described herein, is capable of crossing the blood-brain barrier. In embodiments the virus particle, e.g., as described herein, e.g., comprising a variant capsid polypeptide described herein, exhibits increased crossing of the blood-brain barrier relative to a virus particle comprising a reference capsid polypeptide, e.g., a reference capsid polypeptide of SEQ ID NO:1. In embodiments, the virus particle, e.g., as described herein, e.g., comprising a variant capsid polypeptide described herein, exhibits increased transduction of neurons, astrocytes, glial cells, or combinations thereof, relative to a virus particle comprising a reference capsid polypeptide, e.g., a reference capsid polypeptide of SEQ ID NO:1.

In some embodiments, a viral particle comprising the variant capsid polypeptide, e.g., the variant capsid polypeptide described herein, exhibits improved properties, e.g., improved biodistribution, transduction and/or production. Unless indicated otherwise, improvement rates are presented as fold-improvement over the rates exhibited by a virus particle comprising capsid polypeptides of SEQ ID NO:1. In some embodiments, improvement means an increase, e.g., in the case of CNS biodistribution or CNS transduction. In other embodiments, improvement means a decrease, e.g., in the case of liver biodistribution or liver transduction. A virus particle having increased biodistribution or transduction in target cells or target tissue types, e.g., CNS and/or skeletal muscle, and/or decreased biodistribution or transduction in off-target cells or off-target tissue types, e.g., PNS, liver and/or spleen, may have improved specificity for a target cell or target tissue type. This improvement may be beneficial in the use of the viral particle to deliver a therapeutic transgene to the target cell or target tissue type in a subject afflicted with a disease affecting the target cell or target tissue type, for example.

In some embodiments, one or more improved properties (e.g., increased or decreased biodistribution and/or transduction) is exhibited in a mammal, e.g., a primate, e.g., a human. In embodiments, the increased or decreased biodistribution and/or transduction is exhibited upon administration of the virus particle or pharmaceutical composition comprising the virus particle, e.g., as described herein, by systemic administration, e.g., intravenous administration.

In some embodiments, a viral particle comprising the variant capsid polypeptide exhibits an improvement in one or more categories as defined below.

In some embodiments, the viral particle exhibits an improvement in Category A (CNS biodistribution) and Category B (CNS transduction). These improvements may be beneficial in the use of the viral particle to deliver a therapeutic transgene to the CNS in a subject afflicted with a disease affecting the CNS, for example. In some embodiments, the viral particle exhibits an improvement in Category A (CNS biodistribution) and Category B (CNS transduction), and one or more of Categories C (PNS biodistribution and/or transduction), D (liver biodistribution and/or transduction) and E (spleen biodistribution and/or transduction). In some embodiments, the viral particle exhibits an improvement in Category A (CNS biodistribution), Category B (CNS transduction), and Category D (liver biodistribution and/or transduction), and optionally exhibits an improvement in Category C (PNS biodistribution and/or transduction) and/or E (spleen biodistribution and/or transduction). Optionally, the viral particle may also exhibit an improvement in Category F (skeletal muscle biodistribution) and/or Category G (skeletal muscle transduction), e.g., to deliver a therapeutic transgene to both CNS and muscle in a subject afflicted with a disease affecting both CNS and skeletal muscle tissue (e.g., SMA, multiple sclerosis, Amyotrophic lateral sclerosis (ALS), Ataxia, Becker muscular dystrophy, Charcot-Marie-Tooth disease, Dystonias, Friedreich's ataxia, Glycogen storage disease II, Kennedy Disease, Lambert-Eaton Myasthenic Syndrome, Mitochondrial DNA depletion syndromes, Muscle-Eye-Brain Disease, Neuromyotonia, Periodic Paralyses, juvenile Primary Lateral Sclerosis, Progressive external ophtalmoplegia, Spastic Paraplegias, congenital Stiff-Person Syndrome, Tardive Dyskinesia, Werdnig-Hoffman Disease, or X-Linked Spinal and Bulbar Muscular Atrophy).

In further embodiments, a viral particle comprising the variant capsid polypeptide exhibits an improvement in Category F (skeletal muscle biodistribution) and/or Category G (skeletal muscle transduction). In some embodiments, the viral particle exhibits an improvement in both Category F (skeletal muscle biodistribution) and Category G (skeletal muscle transduction). These improvements may be beneficial in the use of the viral particle to deliver a therapeutic transgene to the skeletal muscle tissue in a subject afflicted with a disease affecting the skeletal muscle, for example. In some embodiments, the viral particle exhibits an improvement in Category F (skeletal muscle biodistribution) and/or Category G (skeletal muscle transduction), together with one or more of Categories C (PNS biodistribution and/or transduction), D (liver biodistribution and/or transduction) and E (spleen biodistribution and/or transduction). In some embodiments, the viral particle exhibits an improvement in Category F (skeletal muscle biodistribution) and/or Category G (skeletal muscle transduction) and an improvement in Category D (liver biodistribution and/or transduction), and optionally further exhibits an improvement in Category C (PNS biodistribution and/or transduction) and/or E (spleen biodistribution and/or transduction).

Category A (CNS Biodistribution): In some aspects of the disclosure, a viral particle comprising the variant capsid polypeptide, e.g., the variant capsid polypeptide described herein, exhibits increased CNS biodistribution as compared to a viral particle with the wild-type capsid polypeptide (SEQ ID NO:1). In some embodiments, the increased CNS biodistribution comprises increased brain biodistribution and/or spinal cord biodistribution. In some embodiments, increased brain biodistribution is aggregated brain biodistribution, or biodistribution in a particular brain tissue such as brain stem, basal ganglia, cerebellum, forebrain, hippocampus, midbrain, or temporal cortex, in each case measured with a CNS-specific promoter such as hSyn, with a constitutive promoter such as Cbh, or as an aggregate of a CNS-specific promoter such as hSyn and a constitutive promoter such as Cbh). In some embodiments, the increased biodistribution is increased aggregated brain biodistribution using a Cbh or hSyn promoter and/or is as defined in any one of embodiments A-1 through A-30.

Embodiment A-1: In an embodiment of Category A, the biodistribution is about (or at least about) 5 times better relative to a virus particle comprising a variant capsid polypeptide having a reference sequence, e.g., having the wild-type capsid protein, e.g., having capsid polypeptides of SEQ ID NO:1.

Embodiment A-2: In an embodiment of Category A, the biodistribution is about (or at least about) 10 times better relative to a virus particle comprising a variant capsid polypeptide having a reference sequence, e.g., having the wild-type capsid protein, e.g., having capsid polypeptides of SEQ ID NO:1.

Embodiment A-3: In an embodiment of Category A, the biodistribution is about (or at least about) 15 times better relative to a virus particle comprising a variant capsid polypeptide having a reference sequence, e.g., having the wild-type capsid protein, e.g., having capsid polypeptides of SEQ ID NO:1.

Embodiment A-4: In an embodiment of Category A, the biodistribution is about (or at least about) 20 times better relative to a virus particle comprising a variant capsid polypeptide having a reference sequence, e.g., having the wild-type capsid protein, e.g., having capsid polypeptides of SEQ ID NO:1.

Embodiment A-5: In an embodiment of Category A, the biodistribution is about (or at least about) 25 times better relative to a virus particle comprising a variant capsid polypeptide having a reference sequence, e.g., having the wild-type capsid protein, e.g., having capsid polypeptides of SEQ ID NO:1.

Embodiment A-6: In an embodiment of Category A, the biodistribution is about (or at least about) 30 times better relative to a virus particle comprising a variant capsid polypeptide having a reference sequence, e.g., having the wild-type capsid protein, e.g., having capsid polypeptides of SEQ ID NO:1.

Embodiment A-7: In an embodiment of Category A, the biodistribution is about (or at least about) 35 times better relative to a virus particle comprising a variant capsid polypeptide having a reference sequence, e.g., having the wild-type capsid protein, e.g., having capsid polypeptides of SEQ ID NO:1.

Embodiment A-8: In an embodiment of Category A, the biodistribution is about (or at least about) 40 times better relative to a virus particle comprising a variant capsid polypeptide having a reference sequence, e.g., having the wild-type capsid protein, e.g., having capsid polypeptides of SEQ ID NO:1.

Embodiment A-9: In an embodiment of Category A, the biodistribution is about (or at least about) 50 times better relative to a virus particle comprising a variant capsid polypeptide having a reference sequence, e.g., having the wild-type capsid protein, e.g., having capsid polypeptides of SEQ ID NO:1.

Embodiment A-10: In an embodiment of Category A, the biodistribution is about (or at least about) 60 times better relative to a virus particle comprising a variant capsid polypeptide having a reference sequence, e.g., having the wild-type capsid protein, e.g., having capsid polypeptides of SEQ ID NO:1.

Embodiment A-11: In an embodiment of Category A, the biodistribution is about (or at least about) 70 times better relative to a virus particle comprising a variant capsid polypeptide having a reference sequence, e.g., having the wild-type capsid protein, e.g., having capsid polypeptides of SEQ ID NO:1.

Embodiment A-12: In an embodiment of Category A, the biodistribution is about (or at least about) 80 times better relative to a virus particle comprising a variant capsid polypeptide having a reference sequence, e.g., having the wild-type capsid protein, e.g., having capsid polypeptides of SEQ ID NO:1.

Embodiment A-13: In an embodiment of Category A, the biodistribution is about (or at least about) 90 times better relative to a virus particle comprising a variant capsid polypeptide having a reference sequence, e.g., having the wild-type capsid protein, e.g., having capsid polypeptides of SEQ ID NO:1.

Embodiment A-14: In an embodiment of Category A, the biodistribution is about (or at least about) 100 times better relative to a virus particle comprising a variant capsid polypeptide having a reference sequence, e.g., having the wild-type capsid protein, e.g., having capsid polypeptides of SEQ ID NO:1.

In some embodiments, the improved biodistribution is in a range bounded by any two values set forth in embodiments A-1 to A-14. Exemplary ranges are set forth in embodiments A-15 to A-30 below.

Embodiment A-15: In an embodiment of Category A, the biodistribution ranges between about 5 and about 100 times better relative to a virus particle comprising a variant capsid polypeptide having a reference sequence, e.g., having the wild-type capsid protein, e.g., having capsid polypeptides of SEQ ID NO:1.

Embodiment A-16: In an embodiment of Category A, the biodistribution ranges between about 5 and about 80 times better relative to a virus particle comprising a variant capsid polypeptide having a reference sequence, e.g., having the wild-type capsid protein, e.g., having capsid polypeptides of SEQ ID NO:1.

Embodiment A-17: In an embodiment of Category A, the biodistribution ranges between about 5 and about 90 times better relative to a virus particle comprising a variant capsid polypeptide having a reference sequence, e.g., having the wild-type capsid protein, e.g., having capsid polypeptides of SEQ ID NO:1.

Embodiment A-18: In an embodiment of Category A, the biodistribution ranges between about 5 and about 60 times better relative to a virus particle comprising a variant capsid polypeptide having a reference sequence, e.g., having the wild-type capsid protein, e.g., having capsid polypeptides of SEQ ID NO:1.

Embodiment A-19: In an embodiment of Category A, the biodistribution ranges between about 10 and about 100 times better relative to a virus particle comprising a variant capsid polypeptide having a reference sequence, e.g., having the wild-type capsid protein, e.g., having capsid polypeptides of SEQ ID NO:1.

Embodiment A-20: In an embodiment of Category A, the biodistribution ranges between about 10 and about 80 times better relative to a virus particle comprising a variant capsid polypeptide having a reference sequence, e.g., having the wild-type capsid protein, e.g., having capsid polypeptides of SEQ ID NO:1.

Embodiment A-21: In an embodiment of Category A, the biodistribution ranges between about 10 and about 60 times better relative to a virus particle comprising a variant capsid polypeptide having a reference sequence, e.g., having the wild-type capsid protein, e.g., having capsid polypeptides of SEQ ID NO:1.

Embodiment A-22: In an embodiment of Category A, the biodistribution ranges between about 15 and about 90 times better relative to a virus particle comprising a variant capsid polypeptide having a reference sequence, e.g., having the wild-type capsid protein, e.g., having capsid polypeptides of SEQ ID NO:1.

Embodiment A-23: In an embodiment of Category A, the biodistribution ranges between about 15 and about 70 times better relative to a virus particle comprising a variant capsid polypeptide having a reference sequence, e.g., having the wild-type capsid protein, e.g., having capsid polypeptides of SEQ ID NO:1.

Embodiment A-24: In an embodiment of Category A, the biodistribution ranges between about 15 and about 50 times better relative to a virus particle comprising a variant capsid polypeptide having a reference sequence, e.g., having the wild-type capsid protein, e.g., having capsid polypeptides of SEQ ID NO:1.

Embodiment A-25: In an embodiment of Category A, the biodistribution ranges between about 20 and about 60 times better relative to a virus particle comprising a variant capsid polypeptide having a reference sequence, e.g., having the wild-type capsid protein, e.g., having capsid polypeptides of SEQ ID NO:1.

Embodiment A-26: In an embodiment of Category A, the biodistribution ranges between about 20 and about 40 times better relative to a virus particle comprising a variant capsid polypeptide having a reference sequence, e.g., having the wild-type capsid protein, e.g., having capsid polypeptides of SEQ ID NO:1

Embodiment A-27: In an embodiment of Category A, the biodistribution ranges between about 30 and about 50 times better relative to a virus particle comprising a variant capsid polypeptide having a reference sequence, e.g., having the wild-type capsid protein, e.g., having capsid polypeptides of SEQ ID NO:1.

Embodiment A-28: In an embodiment of Category A, the biodistribution ranges between about 10 and about 40 times better relative to a virus particle comprising a variant capsid polypeptide having a reference sequence, e.g., having the wild-type capsid protein, e.g., having capsid polypeptides of SEQ ID NO:1.

Embodiment A-29: In an embodiment of Category A, the biodistribution ranges between about 35 and about 60 times better relative to a virus particle comprising a variant capsid polypeptide having a reference sequence, e.g., having the wild-type capsid protein, e.g., having capsid polypeptides of SEQ ID NO:1.

Embodiment A-30: In an embodiment of Category A, the biodistribution ranges between about 25 and about 70 times better relative to a virus particle comprising a variant capsid polypeptide having a reference sequence, e.g., having the wild-type capsid protein, e.g., having capsid polypeptides of SEQ ID NO:1.

Category B (CNS Transduction): In some aspects of the disclosure, a viral particle comprising the variant capsid polypeptide, e.g., the variant capsid polypeptide described herein, exhibits increased CNS transduction as compared to a viral particle with the wild-type capsid polypeptide (SEQ ID NO:1). In some embodiments, the increased CNS transduction comprises increased brain transduction and/or spinal cord transduction. In some embodiments, increased brain transduction is aggregated brain transduction, or transduction in a particular brain tissue such as brain stem, basal ganglia, cerebellum, forebrain, hippocampus, midbrain, or temporal cortex, in each case measured with a CNS-specific promoter such as hSyn, with a constitutive promoter such as Cbh, or as an aggregate of a CNS-specific promoter such as hSyn and a constitutive promoter such as Cbh. In some embodiments, the increased transduction is increased aggregated brain transduction using a Cbh or hSyn promoter and/or is as defined in any one of embodiments B-1 through B-32.

Embodiment B-1: In an embodiment of Category B, the transduction is about (or at least about) 10 times better relative to a virus particle comprising a variant capsid polypeptide having a reference sequence, e.g., having the wild-type capsid protein, e.g., having capsid polypeptides of SEQ ID NO:1.

Embodiment B-2: In an embodiment of Category B, the transduction is about (or at least about) 25 times better relative to a virus particle comprising a variant capsid polypeptide having a reference sequence, e.g., having the wild-type capsid protein, e.g., having capsid polypeptides of SEQ ID NO:1.

Embodiment B-3: In an embodiment of Category B, the transduction is about (or at least about) 50 times better relative to a virus particle comprising a variant capsid polypeptide having a reference sequence, e.g., having the wild-type capsid protein, e.g., having capsid polypeptides of SEQ ID NO:1.

Embodiment B-4: In an embodiment of Category B, the transduction is about (or at least about) 75 times better relative to a virus particle comprising a variant capsid polypeptide having a reference sequence, e.g., having the wild-type capsid protein, e.g., having capsid polypeptides of SEQ ID NO:1.

Embodiment B-5: In an embodiment of Category B, the transduction is about (or at least about) 100 times better relative to a virus particle comprising a variant capsid polypeptide having a reference sequence, e.g., having the wild-type capsid protein, e.g., having capsid polypeptides of SEQ ID NO:1.

Embodiment B-6: In an embodiment of Category B, the transduction is about (or at least about) 125 times better relative to a virus particle comprising a variant capsid polypeptide having a reference sequence, e.g., having the wild-type capsid protein, e.g., having capsid polypeptides of SEQ ID NO:1.

Embodiment B-7: In an embodiment of Category B, the transduction is about (or at least about) 150 times better relative to a virus particle comprising a variant capsid polypeptide having a reference sequence, e.g., having the wild-type capsid protein, e.g., having capsid polypeptides of SEQ ID NO:1.

Embodiment B-8: In an embodiment of Category B, the transduction is about (or at least about) 175 times better relative to a virus particle comprising a variant capsid polypeptide having a reference sequence, e.g., having the wild-type capsid protein, e.g., having capsid polypeptides of SEQ ID NO:1.

Embodiment B-9: In an embodiment of Category B, the transduction is about (or at least about) 200 times better relative to a virus particle comprising a variant capsid polypeptide having a reference sequence, e.g., having the wild-type capsid protein, e.g., having capsid polypeptides of SEQ ID NO:1.

Embodiment B-10: In an embodiment of Category B, the transduction is about (or at least about) 225 times better relative to a virus particle comprising a variant capsid polypeptide having a reference sequence, e.g., having the wild-type capsid protein, e.g., having capsid polypeptides of SEQ ID NO:1.

Embodiment B-11: In an embodiment of Category B, the transduction is about (or at least about) 250 times better relative to a virus particle comprising a variant capsid polypeptide having a reference sequence, e.g., having the wild-type capsid protein, e.g., having capsid polypeptides of SEQ ID NO:1.

Embodiment B-12: In an embodiment of Category B, the transduction is about (or at least about) 275 times better relative to a virus particle comprising a variant capsid polypeptide having a reference sequence, e.g., having the wild-type capsid protein, e.g., having capsid polypeptides of SEQ ID NO:1.

Embodiment B-13: In an embodiment of Category B, the transduction is about (or at least about) 300 times better relative to a virus particle comprising a variant capsid polypeptide having a reference sequence, e.g., having the wild-type capsid protein, e.g., having capsid polypeptides of SEQ ID NO:1.

In some embodiments, the improved transduction is in a range bounded by any two values set forth in embodiments B-1 to B-13. Exemplary ranges are set forth in embodiments B-14 to B-32 below.

Embodiment B-14: In an embodiment of Category B, the transduction ranges between about 10 and about 300 times better relative to a virus particle comprising a variant capsid polypeptide having a reference sequence, e.g., having the wild-type capsid protein, e.g., having capsid polypeptides of SEQ ID NO:1.

Embodiment B-15: In an embodiment of Category B, the transduction ranges between about 25 and about 300 times better relative to a virus particle comprising a variant capsid polypeptide having a reference sequence, e.g., having the wild-type capsid protein, e.g., having capsid polypeptides of SEQ ID NO:1.

Embodiment B-16: In an embodiment of Category B, the transduction ranges between about 50 and about 300 times better relative to a virus particle comprising a variant capsid polypeptide having a reference sequence, e.g., having the wild-type capsid protein, e.g., having capsid polypeptides of SEQ ID NO:1.

Embodiment B-17: In an embodiment of Category B, the transduction ranges between about 75 and about 300 times better relative to a virus particle comprising a variant capsid polypeptide having a reference sequence, e.g., having the wild-type capsid protein, e.g., having capsid polypeptides of SEQ ID NO:1.

Embodiment B-18: In an embodiment of Category B, the transduction ranges between about 100 and about 300 times better relative to a virus particle comprising a variant capsid polypeptide having a reference sequence, e.g., having the wild-type capsid protein, e.g., having capsid polypeptides of SEQ ID NO:1.

Embodiment B-19: In an embodiment of Category B, the transduction ranges between about 10 and about 250 times better relative to a virus particle comprising a variant capsid polypeptide having a reference sequence, e.g., having the wild-type capsid protein, e.g., having capsid polypeptides of SEQ ID NO:1.

Embodiment B-20: In an embodiment of Category B, the transduction ranges between about 25 and about 250 times better relative to a virus particle comprising a variant capsid polypeptide having a reference sequence, e.g., having the wild-type capsid protein, e.g., having capsid polypeptides of SEQ ID NO:1.

Embodiment B-21: In an embodiment of Category B, the transduction ranges between about 50 and about 250 times better relative to a virus particle comprising a variant capsid polypeptide having a reference sequence, e.g., having the wild-type capsid protein, e.g., having capsid polypeptides of SEQ ID NO:1.

Embodiment B-22: In an embodiment of Category B, the transduction ranges between about 75 and about 250 times better relative to a virus particle comprising a variant capsid polypeptide having a reference sequence, e.g., having the wild-type capsid protein, e.g., having capsid polypeptides of SEQ ID NO:1.

Embodiment B-23: In an embodiment of Category B, the transduction ranges between about 100 and about 250 times better relative to a virus particle comprising a variant capsid polypeptide having a reference sequence, e.g., having the wild-type capsid protein, e.g., having capsid polypeptides of SEQ ID NO:1.

Embodiment B-24: In an embodiment of Category B, the transduction ranges between about 25 and about 225 times better relative to a virus particle comprising a variant capsid polypeptide having a reference sequence, e.g., having the wild-type capsid protein, e.g., having capsid polypeptides of SEQ ID NO:1.

Embodiment B-25: In an embodiment of Category B, the transduction ranges between about 25 and about 225 times better relative to a virus particle comprising a variant capsid polypeptide having a reference sequence, e.g., having the wild-type capsid protein, e.g., having capsid polypeptides of SEQ ID NO:1.

Embodiment B-26: In an embodiment of Category B, the transduction ranges between about 50 and about 225 times better relative to a virus particle comprising a variant capsid polypeptide having a reference sequence, e.g., having the wild-type capsid protein, e.g., having capsid polypeptides of SEQ ID NO:1

Embodiment B-27: In an embodiment of Category B, the transduction ranges between about 75 and about 225 times better relative to a virus particle comprising a variant capsid polypeptide having a reference sequence, e.g., having the wild-type capsid protein, e.g., having capsid polypeptides of SEQ ID NO:1.

Embodiment B-28: In an embodiment of Category B, the transduction ranges between about 75 and about 225 times better relative to a virus particle comprising a variant capsid polypeptide having a reference sequence, e.g., having the wild-type capsid protein, e.g., having capsid polypeptides of SEQ ID NO:1.

Embodiment B-29: In an embodiment of Category B, the transduction ranges between about 25 and about 200 times better relative to a virus particle comprising a variant capsid polypeptide having a reference sequence, e.g., having the wild-type capsid protein, e.g., having capsid polypeptides of SEQ ID NO:1.

Embodiment B-30: In an embodiment of Category B, the transduction ranges between about 50 and about 200 times better relative to a virus particle comprising a variant capsid polypeptide having a reference sequence, e.g., having the wild-type capsid protein, e.g., having capsid polypeptides of SEQ ID NO:1.

Embodiment B-31: In an embodiment of Category B, the transduction ranges between about 75 and about 200 times better relative to a virus particle comprising a variant capsid polypeptide having a reference sequence, e.g., having the wild-type capsid protein, e.g., having capsid polypeptides of SEQ ID NO:1.

Embodiment B-32: In an embodiment of Category B, the transduction ranges between about 100 and about 200 times better relative to a virus particle comprising a variant capsid polypeptide having a reference sequence, e.g., having the wild-type capsid protein, e.g., having capsid polypeptides of SEQ ID NO:1.

Category C (Peripheral Nervous System (“PNS”) Biodistribution and/or Transduction): In some aspects of the disclosure, a viral particle comprising the variant capsid polypeptide, e.g., the variant capsid polypeptide described herein, exhibits similar or reduced PNS biodistribution and/or transduction as compared to a viral particle with the wild-type capsid polypeptide (SEQ ID NO:1). In some embodiments, the PNS biodistribution and/or transduction is measured with a CNS-specific promoter such as hSyn, with a constitutive promoter such as Cbh, or as an aggregate of a CNS-specific promoter such as hSyn and a constitutive promoter such as Cbh. In some embodiments, the PNS biodistribution and/or transduction is DRG biodistribution and/or transduction and/or is as defined in any one of embodiments C-1 through C-22.

Embodiment C-1: In an embodiment of Category C, the PNS biodistribution and/or transduction is about (or no more than about) 10 times increased relative to a virus particle comprising a variant capsid polypeptide having a reference sequence, e.g., having the wild-type capsid protein, e.g., having capsid polypeptides of SEQ ID NO:1.

Embodiment C-2: In an embodiment of Category C, the PNS biodistribution and/or transduction is about (or no more than about) 5 times increased relative to a virus particle comprising a variant capsid polypeptide having a reference sequence, e.g., having the wild-type capsid protein, e.g., having capsid polypeptides of SEQ ID NO:1.

Embodiment C-3: In an embodiment of Category C, the PNS biodistribution and/or transduction is about (or no more than about) 2 times increased relative to a virus particle comprising a variant capsid polypeptide having a reference sequence, e.g., having the wild-type capsid protein, e.g., having capsid polypeptides of SEQ ID NO:1.

Embodiment C-4: In an embodiment of Category C, the PNS biodistribution and/or transduction is about (or no more than about) 1.5 times increased relative to a virus particle comprising a variant capsid polypeptide having a reference sequence, e.g., having the wild-type capsid protein, e.g., having capsid polypeptides of SEQ ID NO:1.

Embodiment C-5: In an embodiment of Category C, the PNS biodistribution and/or transduction is about (or no more than about) 1 times a virus particle comprising a variant capsid polypeptide having a reference sequence, e.g., having the wild-type capsid protein, e.g., having capsid polypeptides of SEQ ID NO:1.

Embodiment C-6: In an embodiment of Category C, the PNS biodistribution and/or transduction is about (or no more than about) 0.9 times the PNS biodistribution and/or transduction of a virus particle comprising a variant capsid polypeptide having a reference sequence, e.g., having the wild-type capsid protein, e.g., having capsid polypeptides of SEQ ID NO:1.

Embodiment C-7: In an embodiment of Category C, the PNS biodistribution and/or transduction is about (or no more than about) 0.8 times the PNS biodistribution and/or transduction of a virus particle comprising a variant capsid polypeptide having a reference sequence, e.g., having the wild-type capsid protein, e.g., having capsid polypeptides of SEQ ID NO:1.

Embodiment C-8: In an embodiment of Category C, the PNS biodistribution and/or transduction is about (or no more than) 0.7 times the PNS biodistribution and/or transduction of a virus particle comprising a variant capsid polypeptide having a reference sequence, e.g., having the wild-type capsid protein, e.g., having capsid polypeptides of SEQ ID NO:1.

Embodiment C-9: In an embodiment of Category C, the PNS biodistribution and/or transduction is about (or no more than) 0.6 times the PNS biodistribution and/or transduction of a virus particle comprising a variant capsid polypeptide having a reference sequence, e.g., having the wild-type capsid protein, e.g., having capsid polypeptides of SEQ ID NO:1.

Embodiment C-10: In an embodiment of Category C, the PNS biodistribution and/or transduction is about (or no more than) 0.6 times the PNS biodistribution and/or transduction of a virus particle comprising a variant capsid polypeptide having a reference sequence, e.g., having the wild-type capsid protein, e.g., having capsid polypeptides of SEQ ID NO:1.

In some embodiments, the improved PNS biodistribution and/or transduction is in a range bounded by any two values set forth in embodiments C-1 to C-10. Exemplary ranges are set forth in embodiments C-11 to C-22 below.

Embodiment C-11: In an embodiment of Category C, the PNS biodistribution and/or transduction ranges between about 0.5 and about 10 times the PNS biodistribution and/or transduction of capsid polypeptide having a reference sequence, e.g., having the wild-type capsid protein, e.g., having capsid polypeptides of SEQ ID NO:1.

Embodiment C-12: In an embodiment of Category C, the PNS biodistribution and/or transduction ranges between about 0.5 and about 5 times the PNS biodistribution and/or transduction of capsid polypeptide having a reference sequence, e.g., having the wild-type capsid protein, e.g., having capsid polypeptides of SEQ ID NO:1.

Embodiment C-13: In an embodiment of Category C, the PNS biodistribution and/or transduction ranges between about 0.6 and about 10 times the PNS biodistribution and/or transduction of capsid polypeptide having a reference sequence, e.g., having the wild-type capsid protein, e.g., having capsid polypeptides of SEQ ID NO:1.

Embodiment C-14: In an embodiment of Category C, the PNS biodistribution and/or transduction ranges between about 0.6 and about 5 times the PNS biodistribution and/or transduction of capsid polypeptide having a reference sequence, e.g., having the wild-type capsid protein, e.g., having capsid polypeptides of SEQ ID NO:1.

Embodiment C-15: In an embodiment of Category C, the PNS biodistribution and/or transduction ranges between about 0.7 and about 10 times the PNS biodistribution and/or transduction of capsid polypeptide having a reference sequence, e.g., having the wild-type capsid protein, e.g., having capsid polypeptides of SEQ ID NO:1.

Embodiment C-16: In an embodiment of Category C, the PNS biodistribution and/or transduction ranges between about 0.7 and about 5 times the PNS biodistribution and/or transduction of capsid polypeptide having a reference sequence, e.g., having the wild-type capsid protein, e.g., having capsid polypeptides of SEQ ID NO:1.

Embodiment C-17: In an embodiment of Category C, the PNS biodistribution and/or transduction ranges between about 0.8 and about 10 times the PNS biodistribution and/or transduction of capsid polypeptide having a reference sequence, e.g., having the wild-type capsid protein, e.g., having capsid polypeptides of SEQ ID NO:1.

Embodiment C-18: In an embodiment of Category C, the PNS biodistribution and/or transduction ranges between about 0.8 and about 5 times the PNS biodistribution and/or transduction of capsid polypeptide having a reference sequence, e.g., having the wild-type capsid protein, e.g., having capsid polypeptides of SEQ ID NO:1.

Embodiment C-19: In an embodiment of Category C, the PNS biodistribution and/or transduction ranges between about 0.9 and about 10 times the PNS biodistribution and/or transduction of capsid polypeptide having a reference sequence, e.g., having the wild-type capsid protein, e.g., having capsid polypeptides of SEQ ID NO:1.

Embodiment C-20: In an embodiment of Category C, the PNS biodistribution and/or transduction ranges between about 0.9 and about 5 times the PNS biodistribution and/or transduction of capsid polypeptide having a reference sequence, e.g., having the wild-type capsid protein, e.g., having capsid polypeptides of SEQ ID NO:1.

Embodiment C-21: In an embodiment of Category C, the PNS biodistribution and/or transduction ranges between about 1 and about 10 times the PNS biodistribution and/or transduction of capsid polypeptide having a reference sequence, e.g., having the wild-type capsid protein, e.g., having capsid polypeptides of SEQ ID NO:1.

Embodiment C-22: In an embodiment of Category C, the PNS biodistribution and/or transduction ranges between about 1 and about 5 times the PNS biodistribution and/or transduction of capsid polypeptide having a reference sequence, e.g., having the wild-type capsid protein, e.g., having capsid polypeptides of SEQ ID NO:1.

Category D (Liver Biodistribution and/or Transduction): In some aspects of the disclosure, a viral particle comprising the variant capsid polypeptide, e.g., the variant capsid polypeptide described herein, exhibits similar or reduced liver biodistribution and/or transduction as compared to a viral particle with the wild-type capsid polypeptide (SEQ ID NO:1). In some embodiments, the liver biodistribution and/or transduction is measured with a CNS-specific promoter such as hSyn, with a constitutive promoter such as Cbh, or as an aggregate of a CNS-specific promoter such as hSyn and a constitutive promoter such as Cbh. In some embodiments, the liver biodistribution and/or transduction is as defined in any one of embodiments D-1 through D-30.

Embodiment D-1: In an embodiment of Category D, the liver biodistribution and/or transduction is about (or no more than about) 1-times the liver biodistribution and/or transduction of a virus particle comprising a variant capsid polypeptide having a reference sequence, e.g., having the wild-type capsid protein, e.g., having capsid polypeptides of SEQ ID NO:1.

Embodiment D-2: In an embodiment of Category D, the liver biodistribution and/or transduction is about (or no more than about) 0.9-times the liver biodistribution and/or transduction of a virus particle comprising a variant capsid polypeptide having a reference sequence, e.g., having the wild-type capsid protein, e.g., having capsid polypeptides of SEQ ID NO:1.

Embodiment D-3: In an embodiment of Category D, the liver biodistribution and/or transduction is about (or no more than about) 0.7-times the liver biodistribution and/or transduction of a virus particle comprising a variant capsid polypeptide having a reference sequence, e.g., having the wild-type capsid protein, e.g., having capsid polypeptides of SEQ ID NO:1.

Embodiment D-4: In an embodiment of Category D, the liver biodistribution and/or transduction is about (or no more than about) 0.6-times the liver biodistribution and/or transduction of a virus particle comprising a variant capsid polypeptide having a reference sequence, e.g., having the wild-type capsid protein, e.g., having capsid polypeptides of SEQ ID NO:1.

Embodiment D-5: In an embodiment of Category D, the liver biodistribution and/or transduction is about (or no more than about) 0.5-times the liver biodistribution and/or transduction of a virus particle comprising a variant capsid polypeptide having a reference sequence, e.g., having the wild-type capsid protein, e.g., having capsid polypeptides of SEQ ID NO:1.

Embodiment D-6: In an embodiment of Category D, the liver biodistribution and/or transduction is about (or no more than about) 0.4-times the liver biodistribution and/or transduction of a virus particle comprising a variant capsid polypeptide having a reference sequence, e.g., having the wild-type capsid protein, e.g., having capsid polypeptides of SEQ ID NO:1.

Embodiment D-7: In an embodiment of Category D, the liver biodistribution and/or transduction is about (or no more than about) 0.3-times the liver biodistribution and/or transduction of a virus particle comprising a variant capsid polypeptide having a reference sequence, e.g., having the wild-type capsid protein, e.g., having capsid polypeptides of SEQ ID NO:1.

Embodiment D-8: In an embodiment of Category D, the liver biodistribution and/or transduction is about (or no more than about) 0.2-times the liver biodistribution and/or transduction of a virus particle comprising a variant capsid polypeptide having a reference sequence, e.g., having the wild-type capsid protein, e.g., having capsid polypeptides of SEQ ID NO:1.

Embodiment D-9: In an embodiment of Category D, the liver biodistribution and/or transduction is about (or no more than about) 0.1-times the liver biodistribution and/or transduction of a virus particle comprising a variant capsid polypeptide having a reference sequence, e.g., having the wild-type capsid protein, e.g., having capsid polypeptides of SEQ ID NO:1.

In some embodiments, the improved liver biodistribution and/or transduction is in a range bounded by any two values set forth in embodiments D-1 to D-9. Exemplary ranges are set forth in embodiments D-10 to D-23 below.

Embodiment D-10: In an embodiment of Category D, the liver biodistribution and/or transduction ranges between about 0.1 and about 1 times the liver biodistribution and/or transduction of capsid polypeptide having a reference sequence, e.g., having the wild-type capsid protein, e.g., having capsid polypeptides of SEQ ID NO:1.

Embodiment D-11: In an embodiment of Category D, the liver biodistribution and/or transduction ranges between about 0.2 and about 1 times the liver biodistribution and/or transduction of capsid polypeptide having a reference sequence, e.g., having the wild-type capsid protein, e.g., having capsid polypeptides of SEQ ID NO:1.

Embodiment D-12: In an embodiment of Category D, the liver biodistribution and/or transduction ranges between about 0.1 and about 0.9 times the liver biodistribution and/or transduction of capsid polypeptide having a reference sequence, e.g., having the wild-type capsid protein, e.g., having capsid polypeptides of SEQ ID NO:1.

Embodiment D-13: In an embodiment of Category D, the liver biodistribution and/or transduction ranges between about 0.2 and about 0.9 times the liver biodistribution and/or transduction of capsid polypeptide having a reference sequence, e.g., having the wild-type capsid protein, e.g., having capsid polypeptides of SEQ ID NO:1.

Embodiment D-14: In an embodiment of Category D, the liver biodistribution and/or transduction ranges between about 0.1 and about 0.8 times the liver biodistribution and/or transduction of capsid polypeptide having a reference sequence, e.g., having the wild-type capsid protein, e.g., having capsid polypeptides of SEQ ID NO:1.

Embodiment D-15: In an embodiment of Category D, the liver biodistribution and/or transduction ranges between about 0.2 and about 0.8 times the liver biodistribution and/or transduction of capsid polypeptide having a reference sequence, e.g., having the wild-type capsid protein, e.g., having capsid polypeptides of SEQ ID NO:1.

Embodiment D-16: In an embodiment of Category D, the liver biodistribution and/or transduction ranges between about 0.1 and about 0.7 times the liver biodistribution and/or transduction of capsid polypeptide having a reference sequence, e.g., having the wild-type capsid protein, e.g., having capsid polypeptides of SEQ ID NO:1.

Embodiment D-17: In an embodiment of Category D, the liver biodistribution and/or transduction ranges between about 0.2 and about 0.7 times the liver biodistribution and/or transduction of capsid polypeptide having a reference sequence, e.g., having the wild-type capsid protein, e.g., having capsid polypeptides of SEQ ID NO:1.

Embodiment D-18: In an embodiment of Category D, the liver biodistribution and/or transduction ranges between about 0.1 and about 0.6 times the liver biodistribution and/or transduction of capsid polypeptide having a reference sequence, e.g., having the wild-type capsid protein, e.g., having capsid polypeptides of SEQ ID NO:1.

Embodiment D-19: In an embodiment of Category D, the liver biodistribution and/or transduction ranges between about 0.2 and about 0.6 times the liver biodistribution and/or transduction of capsid polypeptide having a reference sequence, e.g., having the wild-type capsid protein, e.g., having capsid polypeptides of SEQ ID NO:1.

Embodiment D-20: In an embodiment of Category D, the liver biodistribution and/or transduction ranges between about 0.1 and about 0.5 times the liver biodistribution and/or transduction of capsid polypeptide having a reference sequence, e.g., having the wild-type capsid protein, e.g., having capsid polypeptides of SEQ ID NO:1.

Embodiment D-21: In an embodiment of Category D, the liver biodistribution and/or transduction ranges between about 0.2 and about 0.5 times the liver biodistribution and/or transduction of capsid polypeptide having a reference sequence, e.g., having the wild-type capsid protein, e.g., having capsid polypeptides of SEQ ID NO:1.

Embodiment D-22: In an embodiment of Category D, the liver biodistribution and/or transduction ranges between about 0.1 and about 0.4 times the liver biodistribution and/or transduction of capsid polypeptide having a reference sequence, e.g., having the wild-type capsid protein, e.g., having capsid polypeptides of SEQ ID NO:1.

Embodiment D-23: In an embodiment of Category D, the liver biodistribution and/or transduction ranges between about 0.2 and about 0.4 times the liver biodistribution and/or transduction of capsid polypeptide having a reference sequence, e.g., having the wild-type capsid protein, e.g., having capsid polypeptides of SEQ ID NO:1.

Category E (Spleen Biodistribution and/or Transduction): In some aspects of the disclosure, a viral particle comprising the variant capsid polypeptide, e.g., the variant capsid polypeptide described herein, exhibits similar or reduced spleen biodistribution and/or transduction as compared to a viral particle with the wild-type capsid polypeptide (SEQ ID NO:1). In some embodiments, the spleen biodistribution and/or transduction is measured with a CNS-specific promoter such as hSyn, with a constitutive promoter such as Cbh, or as an aggregate of a CNS-specific promoter such as hSyn and a constitutive promoter such as Cbh. In some embodiments, the spleen biodistribution and/or transduction is as defined in any one of embodiments E-1 through E-30.

Embodiment E-1: In an embodiment of Category E, the spleen biodistribution and/or transduction is about (or no more than about) 10-times the spleen biodistribution and/or transduction of a virus particle comprising a variant capsid polypeptide having a reference sequence, e.g., having the wild-type capsid protein, e.g., having capsid polypeptides of SEQ ID NO:1.

Embodiment E-2: In an embodiment of Category E, the spleen biodistribution and/or transduction is about (or no more than about) 5-times the spleen biodistribution and/or transduction of a virus particle comprising a variant capsid polypeptide having a reference sequence, e.g., having the wild-type capsid protein, e.g., having capsid polypeptides of SEQ ID NO:1.

Embodiment E-3: In an embodiment of Category E, the spleen biodistribution and/or transduction is about (or no more than about) 3-times the spleen biodistribution and/or transduction of a virus particle comprising a variant capsid polypeptide having a reference sequence, e.g., having the wild-type capsid protein, e.g., having capsid polypeptides of SEQ ID NO:1.

Embodiment E-4: In an embodiment of Category E, the spleen biodistribution and/or transduction is about (or no more than about) 2-times the spleen biodistribution and/or transduction of a virus particle comprising a variant capsid polypeptide having a reference sequence, e.g., having the wild-type capsid protein, e.g., having capsid polypeptides of SEQ ID NO:1.

Embodiment E-5: In an embodiment of Category E, the spleen biodistribution and/or transduction is about (or no more than about) 1.5-times the spleen biodistribution and/or transduction of a virus particle comprising a variant capsid polypeptide having a reference sequence, e.g., having the wild-type capsid protein, e.g., having capsid polypeptides of SEQ ID NO:1.

Embodiment E-6: In an embodiment of Category E, the spleen biodistribution and/or transduction is about (or no more than about) 1-times the spleen biodistribution and/or transduction of a virus particle comprising a variant capsid polypeptide having a reference sequence, e.g., having the wild-type capsid protein, e.g., having capsid polypeptides of SEQ ID NO:1.

Embodiment E-7: In an embodiment of Category E, the spleen biodistribution and/or transduction is about (or no more than about) 0.8-times the spleen biodistribution and/or transduction of a virus particle comprising a variant capsid polypeptide having a reference sequence, e.g., having the wild-type capsid protein, e.g., having capsid polypeptides of SEQ ID NO:1.

Embodiment E-8: In an embodiment of Category E, the spleen biodistribution and/or transduction is about (or no more than about) 0.6-times the spleen biodistribution and/or transduction of a virus particle comprising a variant capsid polypeptide having a reference sequence, e.g., having the wild-type capsid protein, e.g., having capsid polypeptides of SEQ ID NO:1.

Embodiment E-9: In an embodiment of Category E, the spleen biodistribution and/or transduction is about (or no more than about) 0.4-times the spleen biodistribution and/or transduction of a virus particle comprising a variant capsid polypeptide having a reference sequence, e.g., having the wild-type capsid protein, e.g., having capsid polypeptides of SEQ ID NO:1.

Embodiment E-10: In an embodiment of Category E, the spleen biodistribution and/or transduction is about (or no more than about) 0.2-times the spleen biodistribution and/or transduction of a virus particle comprising a variant capsid polypeptide having a reference sequence, e.g., having the wild-type capsid protein, e.g., having capsid polypeptides of SEQ ID NO:1.

In some embodiments, the improved spleen biodistribution and/or transduction is in a range bounded by any two values set forth in embodiments E-1 to E-10. Exemplary ranges are set forth in embodiments E-11 to E-22 below.

Embodiment E-11: In an embodiment of Category E, the spleen biodistribution and/or transduction ranges between about 0.2 and about 10 times the spleen biodistribution and/or transduction of capsid polypeptide having a reference sequence, e.g., having the wild-type capsid protein, e.g., having capsid polypeptides of SEQ ID NO:1.

Embodiment E-12: In an embodiment of Category E, the spleen biodistribution and/or transduction ranges between about 0.2 and about 5 times the spleen biodistribution and/or transduction of capsid polypeptide having a reference sequence, e.g., having the wild-type capsid protein, e.g., having capsid polypeptides of SEQ ID NO:1.

Embodiment E-13: In an embodiment of Category E, the spleen biodistribution and/or transduction ranges between about 0.2 and about 3 times the spleen biodistribution and/or transduction of capsid polypeptide having a reference sequence, e.g., having the wild-type capsid protein, e.g., having capsid polypeptides of SEQ ID NO:1.

Embodiment E-14: In an embodiment of Category E, the spleen biodistribution and/or transduction ranges between about 0.2 and about 2 times the spleen biodistribution and/or transduction of capsid polypeptide having a reference sequence, e.g., having the wild-type capsid protein, e.g., having capsid polypeptides of SEQ ID NO:1.

Embodiment E-15: In an embodiment of Category E, the spleen biodistribution and/or transduction ranges between about 0.2 and about 1.5 times the spleen biodistribution and/or transduction of capsid polypeptide having a reference sequence, e.g., having the wild-type capsid protein, e.g., having capsid polypeptides of SEQ ID NO:1.

Embodiment E-16: In an embodiment of Category E, the spleen biodistribution and/or transduction ranges between about 0.2 and about 1 times the spleen biodistribution and/or transduction of capsid polypeptide having a reference sequence, e.g., having the wild-type capsid protein, e.g., having capsid polypeptides of SEQ ID NO:1.

Embodiment E-17: In an embodiment of Category E, the spleen biodistribution and/or transduction ranges between about 0.4 and about 10 times the spleen biodistribution and/or transduction of capsid polypeptide having a reference sequence, e.g., having the wild-type capsid protein, e.g., having capsid polypeptides of SEQ ID NO:1.

Embodiment E-18: In an embodiment of Category E, the spleen biodistribution and/or transduction ranges between about 0.4 and about 5 times the spleen biodistribution and/or transduction of capsid polypeptide having a reference sequence, e.g., having the wild-type capsid protein, e.g., having capsid polypeptides of SEQ ID NO:1.

Embodiment E-19: In an embodiment of Category E, the spleen biodistribution and/or transduction ranges between about 0.4 and about 3 times the spleen biodistribution and/or transduction of capsid polypeptide having a reference sequence, e.g., having the wild-type capsid protein, e.g., having capsid polypeptides of SEQ ID NO:1.

Embodiment E-20: In an embodiment of Category E, the spleen biodistribution and/or transduction ranges between about 0 4 and about 2 times the spleen biodistribution and/or transduction of capsid polypeptide having a reference sequence, e.g., having the wild-type capsid protein, e.g., having capsid polypeptides of SEQ ID NO:1.

Embodiment E-21: In an embodiment of Category E, the spleen biodistribution and/or transduction ranges between about 0.4 and about 1.5 times the spleen biodistribution and/or transduction of capsid polypeptide having a reference sequence, e.g., having the wild-type capsid protein, e.g., having capsid polypeptides of SEQ ID NO:1.

Embodiment E-22: In an embodiment of Category E, the spleen biodistribution and/or transduction ranges between about 0.4 and about 1 times the spleen biodistribution and/or transduction of capsid polypeptide having a reference sequence, e.g., having the wild-type capsid protein, e.g., having capsid polypeptides of SEQ ID NO:1.

Category F (Skeletal Muscle Biodistribution): In some aspects of the disclosure, a viral particle comprising the variant capsid polypeptide, e.g., the variant capsid polypeptide described herein, exhibits increased skeletal muscle biodistribution as compared to a viral particle with the wild-type capsid polypeptide (SEQ ID NO:1). In some embodiments, the increased skeletal muscle biodistribution is measured with a constitutive promoter such as Cbh. In some embodiments, the increased skeletal muscle biodistribution is as defined in any one of embodiments F-1 through F-10.

Embodiment F-1: In an embodiment of Category F, the biodistribution is about (or at least about) 1.5 times better relative to a virus particle comprising a variant capsid polypeptide having a reference sequence, e.g., having the wild-type capsid protein, e.g., having capsid polypeptides of SEQ ID NO:1.

Embodiment F-2: In an embodiment of Category F, the biodistribution is about (or at least about) 2 times better relative to a virus particle comprising a variant capsid polypeptide having a reference sequence, e.g., having the wild-type capsid protein, e.g., having capsid polypeptides of SEQ ID NO:1.

Embodiment F-3: In an embodiment of Category F, the biodistribution is about (or at least about) 3 times better relative to a virus particle comprising a variant capsid polypeptide having a reference sequence, e.g., having the wild-type capsid protein, e.g., having capsid polypeptides of SEQ ID NO:1.

Embodiment F-4: In an embodiment of Category F, the biodistribution is about (or at least about) 4 times better relative to a virus particle comprising a variant capsid polypeptide having a reference sequence, e.g., having the wild-type capsid protein, e.g., having capsid polypeptides of SEQ ID NO:1.

Embodiment F-5: In an embodiment of Category F, the biodistribution is about (or at least about) 5 times better relative to a virus particle comprising a variant capsid polypeptide having a reference sequence, e.g., having the wild-type capsid protein, e.g., having capsid polypeptides of SEQ ID NO:1.

Embodiment F-6: In an embodiment of Category F, the biodistribution is about (or at least about) 6 times better relative to a virus particle comprising a variant capsid polypeptide having a reference sequence, e.g., having the wild-type capsid protein, e.g., having capsid polypeptides of SEQ ID NO:1.

Embodiment F-7: In an embodiment of Category F, the biodistribution is about (or at least about) 7 times better relative to a virus particle comprising a variant capsid polypeptide having a reference sequence, e.g., having the wild-type capsid protein, e.g., having capsid polypeptides of SEQ ID NO:1.

Embodiment F-8: In an embodiment of Category F, the biodistribution is about (or at least about) 8 times better relative to a virus particle comprising a variant capsid polypeptide having a reference sequence, e.g., having the wild-type capsid protein, e.g., having capsid polypeptides of SEQ ID NO:1.

Embodiment F-9: In an embodiment of Category F, the biodistribution is about (or at least about) 9 times better relative to a virus particle comprising a variant capsid polypeptide having a reference sequence, e.g., having the wild-type capsid protein, e.g., having capsid polypeptides of SEQ ID NO:1.

Embodiment F-10: In an embodiment of Category F, the biodistribution is about (or at least about) 10 times better relative to a virus particle comprising a variant capsid polypeptide having a reference sequence, e.g., having the wild-type capsid protein, e.g., having capsid polypeptides of SEQ ID NO:1.

In some embodiments, the improved biodistribution is in a range bounded by any two values set forth in embodiments F-1 to F-10. Exemplary ranges are set forth in embodiments F-11 to F-17 below.

Embodiment F-11: In an embodiment of Category F, the biodistribution ranges between about 1.5 and about 10 times better relative to a virus particle comprising a variant capsid polypeptide having a reference sequence, e.g., having the wild-type capsid protein, e.g., having capsid polypeptides of SEQ ID NO:1.

Embodiment F-12: In an embodiment of Category F, the biodistribution ranges between about 2 and about 10 times better relative to a virus particle comprising a variant capsid polypeptide having a reference sequence, e.g., having the wild-type capsid protein, e.g., having capsid polypeptides of SEQ ID NO:1.

Embodiment F-13: In an embodiment of Category F, the biodistribution ranges between about 3 and about 10 times better relative to a virus particle comprising a variant capsid polypeptide having a reference sequence, e.g., having the wild-type capsid protein, e.g., having capsid polypeptides of SEQ ID NO:1.

Embodiment F-14: In an embodiment of Category F, the biodistribution ranges between about 4 and about 10 times better relative to a virus particle comprising a variant capsid polypeptide having a reference sequence, e.g., having the wild-type capsid protein, e.g., having capsid polypeptides of SEQ ID NO:1.

Embodiment F-15: In an embodiment of Category F, the biodistribution ranges between about 5 and about 10 times better relative to a virus particle comprising a variant capsid polypeptide having a reference sequence, e.g., having the wild-type capsid protein, e.g., having capsid polypeptides of SEQ ID NO:1.

Embodiment F-16: In an embodiment of Category F, the biodistribution ranges between about 6 and about 10 times better relative to a virus particle comprising a variant capsid polypeptide having a reference sequence, e.g., having the wild-type capsid protein, e.g., having capsid polypeptides of SEQ ID NO:1.

Embodiment F-17: In an embodiment of Category F, the biodistribution ranges between about 7 and about 10 times better relative to a virus particle comprising a variant capsid polypeptide having a reference sequence, e.g., having the wild-type capsid protein, e.g., having capsid polypeptides of SEQ ID NO:1.

Category G (Skeletal Muscle Transduction): In some aspects of the disclosure, a viral particle comprising the variant capsid polypeptide, e.g., the variant capsid polypeptide described herein, exhibits increased skeletal muscle transduction as compared to a viral particle with the wild-type capsid polypeptide (SEQ ID NO:1). In some embodiments, the increased skeletal muscle biodistribution is measured with a constitutive promoter such as Cbh. In some embodiments, the increased skeletal muscle transduction is as defined in any one of embodiments G-1 through G-5.

Embodiment G-1: In an embodiment of Category G, the transduction is about (or at least about) 1.5 times better relative to a virus particle comprising a variant capsid polypeptide having a reference sequence, e.g., having the wild-type capsid protein, e.g., having capsid polypeptides of SEQ ID NO:1.

Embodiment G-2: In an embodiment of Category G, the transduction is about (or at least about) 2 times better relative to a virus particle comprising a variant capsid polypeptide having a reference sequence, e.g., having the wild-type capsid protein, e.g., having capsid polypeptides of SEQ ID NO:1.

Embodiment G-3: In an embodiment of Category G, the transduction is about (or at least about) 3 times better relative to a virus particle comprising a variant capsid polypeptide having a reference sequence, e.g., having the wild-type capsid protein, e.g., having capsid polypeptides of SEQ ID NO:1.

Embodiment G-4: In an embodiment of Category G, the transduction is about (or at least about) 4 times better relative to a virus particle comprising a variant capsid polypeptide having a reference sequence, e.g., having the wild-type capsid protein, e.g., having capsid polypeptides of SEQ ID NO:1.

Embodiment G-5: In an embodiment of Category G, the transduction is about (or at least about) 5 times better relative to a virus particle comprising a variant capsid polypeptide having a reference sequence, e.g., having the wild-type capsid protein, e.g., having capsid polypeptides of SEQ ID NO:1.

In some embodiments, the improved transduction is in a range bounded by any two values set forth in embodiments G-1 to G-5. Exemplary ranges are set forth in embodiments G-6 to G-9 below.

Embodiment G-6: In an embodiment of Category G, the transduction ranges between about 1.5 and about 5 times better relative to a virus particle comprising a variant capsid polypeptide having a reference sequence, e.g., having the wild-type capsid protein, e.g., having capsid polypeptides of SEQ ID NO:1.

Embodiment G-7: In an embodiment of Category G, the transduction ranges between about 2 and about 5 times better relative to a virus particle comprising a variant capsid polypeptide having a reference sequence, e.g., having the wild-type capsid protein, e.g., having capsid polypeptides of SEQ ID NO:1.

Embodiment G-8: In an embodiment of Category G, the transduction ranges between about 3 and about 5 times better relative to a virus particle comprising a variant capsid polypeptide having a reference sequence, e.g., having the wild-type capsid protein, e.g., having capsid polypeptides of SEQ ID NO:1.

Embodiment G-9: In an embodiment of Category G, the transduction ranges between about 4 and about 5 times better relative to a virus particle comprising a variant capsid polypeptide having a reference sequence, e.g., having the wild-type capsid protein, e.g., having capsid polypeptides of SEQ ID NO:1.

### 5.5. Methods of Making Compositions Described Herein

The disclosure is directed, in part, to a method of making a dependoparvovirus particle, e.g., a dependoparvovirus particle described herein. In some embodiments, a method of making dependoparvovirus particle comprises providing a cell, cell-free system, or other translation system, comprising a nucleic acid described encoding a variant capsid polypeptide provided for herein, or a polypeptide provided for herein (e.g., a variant capsid polypeptide); and cultivating the cell, cell-free system, or other translation system under conditions suitable for the production of the dependoparvovirus particle, thereby making the dependoparvovirus particle.

In some embodiments, providing a cell comprising a nucleic acid described herein comprises introducing the nucleic acid to the cell, e.g., transfecting or transforming the cell with the nucleic acid. In embodiments, the nucleic acids of the disclosure are situated as a part of any genetic element (vector) which can be delivered to a host cell, e.g., naked DNA, a plasmid, phage, transposon, cosmid, episome, a protein in a non-viral delivery vehicle (e.g., a lipid-based carrier), virus, etc. which transfer the sequences carried thereon. Such a vector can be delivered by any suitable method, including transfection, liposome delivery, electroporation, membrane fusion techniques, viral infection, high velocity DNA-coated pellets, and protoplast fusion. A person of skill in the art possesses the knowledge and skill in nucleic acid manipulation to construct any embodiment of this invention and said skills include genetic engineering, recombinant engineering, and synthetic techniques. See, e.g., Sambrook et al, Molecular Cloning: A Laboratory Manual, Cold Spring Harbor Press, Cold Spring Harbor, NY.

In some embodiments, a vector of the disclosure comprises sequences encoding a dependoparvovirus variant capsid polypeptide as provided for herein or a fragment thereof. In some embodiments, a vector of the disclosure comprises sequences encoding a dependoparvovirus rep protein or a fragment thereof. In some embodiments, such vectors contain both dependoparvovirus cap and rep proteins. In vectors in which both AAV rep and cap are provided, in embodiments, the dependoparvovirus rep and dependoparvovirus cap sequences are both of the same dependoparvovirus species or serotype origin, such as AAV9. Alternatively, the present embodiments also provide vectors in which the rep sequences are from a dependoparvovirus species or serotype which differs from that which is providing the cap sequences. In some embodiments, the rep and cap sequences are expressed from separate sources (e.g., separate vectors, or a host cell genome and a vector). In some embodiments, the rep sequences are fused in frame to cap sequences of a different dependoparvovirus species or serotype to form a chimeric dependoparvovirus vector. In some embodiments, the vectors of the invention further contain a payload, e.g., a minigene comprising a selected transgene, e.g., flanked by dependoparvovirus 5′ ITR and dependoparvovirus 3′ ITR.

The vectors described herein, e.g., a plasmid, are useful for a variety of purposes, but are particularly well suited for use in production of recombinant dependoparvovirus particles comprising dependoparvovirus sequences or a fragment thereof, and in some embodiments, a payload.

In one aspect, the disclosure provides a method of making a dependoparvovirus particle (e.g., a dependoparvovirus B particle, e.g., an AAV9 particle), or a portion thereof. In some embodiments, the method comprises culturing a host cell which contains a nucleic acid sequence encoding a dependoparvovirus variant capsid protein as provided for herein, or fragment thereof; a functional rep gene; a payload, e.g., a minigene comprising dependoparvovirus inverted terminal repeats (ITRs) and a transgene; and sufficient helper functions to promote packaging of the payload, e.g., minigene, into the dependoparvovirus capsid. In embodiments, the components necessary to be cultured in the host cell to package a payload, e.g., minigene, in a dependoparvovirus capsid are provided to the host cell in trans. In some embodiments, any one or more of the required components (e.g., payload (e.g., minigene), rep sequences, cap sequences, and/or helper functions) are provided by a host cell which has been engineered to stably comprise one or more of the required components using methods known to those of skill in the art. In some embodiments, a host cell which has been engineered to stably comprise the required component(s) comprises it under the control of an inducible promoter. In some embodiments, the required component(s) is under the control of a constitutive promoter. Examples of suitable inducible and constitutive promoters are provided herein, and further examples are known to those of skill in the art. In some embodiments, a selected host cell which has been engineered to stably comprise one or more components comprises a component under the control of a constitutive promoter and another component under the control of one or more inducible promoters. For example, a host cell which has been engineered to stably comprise the required components is generated from 293 cells (e.g., which comprise helper functions under the control of a constitutive promoter), which comprises the rep and/or cap proteins under the control of one or more inducible promoters.

In embodiments, the payload (e.g., minigene), rep sequences, cap sequences, and helper functions required for producing a dependoparvovirus particle of the disclosure are delivered to the packaging host cell in the form of any genetic element which transfers the sequences carried thereon (e.g., in a vector or combination of vectors). The genetic element may be delivered by any suitable method, including those described herein. Methods used to construct genetic elements, vectors, and other nucleic acids of the disclosure are known to those with skill and include genetic engineering, recombinant engineering, and synthetic techniques. See, e.g., Sambrook et al, Molecular Cloning: A Laboratory Manual, Cold Spring Harbor Press, Cold Spring Harbor, NY. Similarly, methods of generating rAAV virions are well known and the selection of a suitable method is not a limitation on the present invention. See, e.g., K. Fisher et al, J. Virol, 70:520-532 (1993) and U.S. Pat. No. 5,478,745. Unless otherwise specified, the dependoparvovirus ITRs, and other selected dependoparvovirus components described herein, are readily selected from among any dependoparvovirus species and serotypes, e.g., AAV1, AAV2, AAV3, AAV4, AAV5, AAV6, AAV7, AAV8, AAVrh74, or AAV9. ITRs or other dependoparvovirus components may be readily isolated using techniques available to those of skill in the art from a dependoparvovirus species or serotype. Dependoparvovirus species and serotypes may be isolated or obtained from academic, commercial, or public sources (e.g., the American Type Culture Collection, Manassas, VA). In some embodiments, the dependoparvovirus sequences may be obtained through synthetic or other suitable means by reference to published sequences such as are available in the literature or in databases such as, e.g., GenBank or PubMed

The dependoparvovirus particles (e.g., including a variant capsid polypeptide and, for example, a payload) of the disclosure may be produced using any invertebrate cell type which allows for production of dependoparvovirus or biologic products and which can be maintained in culture. In some embodiments, an insect cell may be used in production of the compositions described herein or in the methods of making a dependoparvovirus particle described herein. For example, an insect cell line used can be from Spodoptera frugiperda, such as Sf9, SF21, SF900+, drosophila cell lines, mosquito cell lines, e.g., Aedes albopictus derived cell lines, domestic silkworm cell lines, e.g., Bombyxmori cell lines, Trichoplusia ni cell lines such as High Five cells or Lepidoptera cell lines such as Ascalapha odorata cell lines. In some embodiments, the insect cells are susceptible to baculovirus infection, including High Five, Sf9, Se301, SeIZD2109, SeUCR1, SP900+, Sf21, BTI-TN-5B1-4, MG-1, Tn368, HzAml, BM-N, Ha2302, Hz2E5 and Ao38.

In some embodiments, the methods of the disclosure can be carried out with any mammalian cell type which allows for replication of dependoparvovirus or production of biologic products, and which can be maintained in culture. In some embodiments, the mammalian cells used can be HEK293, HeLa, CHO, NS0, SP2/0, PER.C6, Vero, RD, BHK, HT 1080, A549, Cos-7, ARPE-19 or MRC-5 cells.

Methods of expressing proteins (e.g., recombinant or heterologous proteins, e.g., dependoparvovirus polypeptides) in insect cells are well documented, as are methods of introducing nucleic acids, such as vectors, e.g., insect-cell compatible vectors, into such cells and methods of maintaining such cells in culture. See, for example, METHODS IN MOLECULAR BIOLOGY, ed. Richard, Humana Press, N J (1995); O'Reilly et al., BACULOVIRUS EXPRESSION VECtoRS, A LABORAtoRY MANUAL, Oxford Univ. Press (1994); Samulski et al., J. Vir. 63:3822-8 (1989); Kajigaya et al., Proc. Nat'l. Acad. Sci. USA 88:4646-50 (1991); Ruffing et al., J. Vir. 66:6922-30 (1992); Kirnbauer et al., Vir. 219:37-44 (1996); Zhao et al., Vir. 272:382-93 (2000); and Samulski et al., U.S. Pat. No. 6,204,059. In some embodiments, a nucleic acid construct encoding dependoparvovirus polypeptides (e.g., a dependoparvovirus genome) in insect cells is an insect cell-compatible vector. An “insect cell-compatible vector” as used herein refers to a nucleic acid molecule capable of productive transformation or transfection of an insect or insect cell. Exemplary biological vectors include plasmids, linear nucleic acid molecules, and recombinant viruses. Any vector can be employed as long as it is insect cell-compatible. The vector may integrate into the insect cell's genome or remain present extra-chromosomally. The vector may be present permanently or transiently, e.g., as an episomal vector. Vectors may be introduced by any means known in the art. Such means include but are not limited to chemical treatment of the cells, electroporation, or infection. In some embodiments, the vector is a baculovirus, a viral vector, or a plasmid.

In some embodiments, a nucleic acid sequence encoding a dependoparvovirus polypeptide is operably linked to regulatory expression control sequences for expression in a specific cell type, such as Sf9 or HEK cells. Techniques known to one skilled in the art for expressing foreign genes in insect host cells or mammalian host cells can be used with the compositions and methods of the disclosure. Methods for molecular engineering and expression of polypeptides in insect cells are described, for example, in Summers and Smith. A Manual of Methods for Baculovirus Vectors and Insect Culture Procedures, Texas Agricultural Experimental Station Bull. No. 7555, College Station, Tex. (1986); Luckow. 1991. In Prokop et al., Cloning and Expression of Heterologous Genes in Insect Cells with Baculovirus Vectors' Recombinant DNA Technology and Applications, 97-152 (1986); King, L. A. and R. D. Possee, The baculovirus expression system, Chapman and Hall, United Kingdom (1992); O'Reilly, D. R., L. K. Miller, V. A. Luckow, Baculovirus Expression Vectors: A Laboratory Manual, New York (1992); W. H. Freeman and Richardson, C. D., Baculovirus Expression Protocols, Methods in Molecular Biology, volume 39 (1995); U.S. Pat. No. 4,745,051; US2003148506; and WO 03/074714. Promoters suitable for transcription of a nucleotide sequence encoding a dependoparvovirus polypeptide include the polyhedron, p10, p35 or IE-1 promoters and further promoters described in the above references are also contemplated.

In some embodiments, providing a cell comprising a nucleic acid described herein comprises acquiring a cell comprising the nucleic acid.

Methods of cultivating cells, cell-free systems, and other translation systems are known to those of skill in the art. In some embodiments, cultivating a cell comprises providing the cell with suitable media and incubating the cell and media for a time suitable to achieve viral particle production.

In some embodiments, a method of making a dependoparvovirus particle further comprises a purification step comprising isolating the dependoparvovirus particle from one or more other components (e.g., from a cell or media component).

In some embodiments, production of the dependoparvovirus particle comprises one or more (e.g., all) of: expression of dependoparvovirus polypeptides, assembly of a dependoparvovirus capsid, expression (e.g., duplication) of a dependoparvovirus genome, and packaging of the dependoparvovirus genome into the dependoparvovirus capsid to produce a dependoparvovirus particle. In some embodiments, production of the dependoparvovirus particle further comprises secretion of the dependoparvovirus particle.

In some embodiments, and as described elsewhere herein, the nucleic acid molecule encoding the variant capsid polypeptide is disposed in a dependoparvovirus genome. In some embodiments, and as described elsewhere herein, the nucleic acid molecule encoding the variant capsid polypeptide is packaged into a dependoparvovirus particle along with the dependoparvovirus genome as part of a method of making a dependoparvovirus particle described herein. In other embodiments, the nucleic acid molecule encoding the variant capsid polypeptide is not packaged into a dependoparvovirus particle made by a method described herein.

In some embodiments, a method of making a dependoparvovirus particle described herein produces a dependoparvovirus particle comprising a payload (e.g., a payload described herein) and the variant capsid polypeptide. In some embodiments, the payload comprises a second nucleic acid (e.g., in addition to the dependoparvovirus genome), and production of the dependoparvovirus particle comprises packaging the second nucleic acid into the dependoparvovirus particle. In some embodiments, a cell, cell-free system, or other translation system for use in a method of making a dependoparvovirus particle comprises the second nucleic acid. In some embodiments, the second nucleic acid comprises an exogenous sequence (e.g., exogenous to the dependoparvovirus, the cell, or to a target cell or subject who will be administered the dependoparvovirus particle). In some embodiments, the exogenous sequence encodes an exogenous polypeptide. In some embodiments, the exogenous sequence encodes a therapeutic product.

In some embodiments, virus particles of the disclosure have a similar production efficiency to viral particles with a reference capsid polypeptide, for example, with the wild-type capsid polypeptide (SEQ ID NO:1). In some embodiments, production efficiency of viral particles of the disclosure is (a) at least 0.1-fold, at least 0.2-fold, at least 0.3-fold, at least 0.4-fold, at least 0.5-fold, at least 0.6-fold, at least 0.7-fold, at least 0.8-fold, or at least 0.9-fold and/or (b) up to 1-fold, e.g., as compared to a viral particle with a reference capsid polypeptide, for example, with the wild-type capsid polypeptide (SEQ ID NO:1), or the production efficiency is within any range bounded by a value in (a) and a value in (b). In some embodiments, production efficiency is at least 0.5-fold as compared to a viral particle with the wild-type capsid polypeptide (SEQ ID NO:1). Production efficiency can be evaluated by producing viral particles having a variant capsid with a genome encoding a unique barcode and a fluorescent reporter gene under the control of a ubiquitous (e.g., CBh) or neuronal cell-type specific promoter (e.g., human synapsin) via transient triple transfection of adherent HEK293T followed by iodixanol gradient purification.

In some embodiments, a nucleic acid or polypeptide described herein is produced by a method known to one of skill in the art. In embodiments, the nucleic acids, polypeptides, and fragments thereof of the disclosure are produced by any suitable means, including recombinant production, chemical synthesis, or other synthetic means. Such production methods are within the knowledge of those of skill in the art and are not a limitation of the present invention.

### 5.6. Applications

The disclosure is directed, in part, to compositions comprising a nucleic acid, polypeptide, or particles described herein. The disclosure is further directed, in part, to methods utilizing a composition, nucleic acid, polypeptide, or particles described herein. As will be apparent based on the disclosure, nucleic acids, polypeptides, particles, and methods disclosed herein have a variety of utilities.

The disclosure is directed, in part, to a vector comprising a nucleic acid described herein, e.g., a nucleic acid encoding a variant capsid polypeptide. Many types of vectors are known to those of skill in the art. In some embodiments, a vector comprises a plasmid. In some embodiments, the vector is an isolated vector, e.g., removed from a cell or other biological components.

The disclosure is directed, in part to a cell, cell-free system, or other translation system, comprising a nucleic acid or vector described herein, e.g., a nucleic acid or vector comprising a nucleic acid molecule encoding a variant capsid polypeptide. In some embodiments, the cell, cell-free system, or other translation system is capable of producing dependoparvovirus particles comprising the variant capsid polypeptides. In some embodiments, the cell, cell-free system, or other translation system comprises a nucleic acid comprising a dependoparvovirus genome or components of a dependoparvovirus genome sufficient to promote production of dependoparvovirus particles comprising the variant capsid polypeptides.

In some embodiments, the cell, cell-free system, or other translation system further comprises one or more non-dependoparvovirus nucleic acid sequences that promote dependoparvovirus particle production and/or secretion. Said sequences are referred to herein as helper sequences. In some embodiments, a helper sequence comprises one or more genes from another virus, e.g., an adenovirus or herpes virus. In some embodiments, the presence of a helper sequence is necessary for production and/or secretion of a dependoparvovirus particle. In some embodiments, a cell, cell-free system, or other translation system comprises a vector, e.g., plasmid, comprising one or more helper sequences.

In some embodiments, a cell, cell-free system, or other translation system comprises a first nucleic acid and a second nucleic acid, wherein the first nucleic acid comprises sequences encoding one or more dependoparvovirus genes (e.g., a Cap gene, a Rep gene, or a complete dependoparvovirus genome) and a helper sequence, and wherein the second nucleic acid comprises a payload. In some embodiments, a cell, cell-free system, or other translation system comprises a first nucleic acid and a second nucleic acid, wherein the first nucleic acid comprises sequences encoding one or more dependoparvovirus genes (e.g., a Cap gene, a Rep gene, or a complete dependoparvovirus genome) and a payload, and wherein the second nucleic acid comprises a helper sequence. In some embodiments, a cell, cell-free system, or other translation system comprises a first nucleic acid and a second nucleic acid, wherein the first nucleic acid comprises a helper sequence and a payload, and wherein the second nucleic acid comprises sequences encoding one or more dependoparvovirus genes (e.g., a Cap gene, a Rep gene, or a complete dependoparvovirus genome). In some embodiments, a cell, cell-free system, or other translation system comprises a first nucleic acid, a second nucleic acid, and a third nucleic acid, wherein the first nucleic acid comprises sequences encoding one or more dependoparvovirus genes (e.g., a Cap gene, a Rep gene, or a complete dependoparvovirus genome), the second nucleic acid comprises a helper sequence, and the third nucleic acid comprises a payload.

In some embodiments, the first nucleic acid, second nucleic acid, and optionally third nucleic acid are situated in separate molecules, e.g., separate vectors or a vector and genomic DNA. In some embodiments, one, two, or all of the first nucleic acid, second nucleic acid, and optionally third nucleic acid are integrated (e.g., stably integrated) into the genome of a cell.

In embodiments, a cell of the disclosure is generated by transfecting a suitable cell with a nucleic acid described herein. In some embodiments, a method of making a dependoparvovirus particle comprising a variant capsid polypeptide as provided for herein or improving a method of making a dependoparvovirus particle comprises providing a cell described herein. In some embodiments, providing a cell comprises transfecting a suitable cell with one or more nucleic acids described herein.

Many types and kinds of cells suitable for use with the nucleic acids and vectors described herein are known in the art. In some embodiments, the cell is a human cell. In some embodiments, the cell is an immortalized cell or a cell from a cell line known in the art. In some embodiments, the cell is an HEK293 cell. In some embodiments, the cell is an HEK293T cell.

### 5.7. Methods of Delivering a Payload

The disclosure is directed, in part, to a method of delivering a payload to a cell, e.g., a cell in a subject or in a sample. In some embodiments, a method of delivering a payload to a cell comprises contacting the cell with a dependoparvovirus particle comprising a variant capsid polypeptide (e.g., described herein) comprising the payload. The disclosure also includes a dependoparvovirus particle comprising a variant capsid polypeptide (e.g., described herein) comprising a payload described herein for use in the methods of delivering a payload described herein. In some embodiments, the dependoparvovirus particle is a dependoparvovirus particle described herein and comprises a payload described herein. In some embodiments, the cell is a CNS cell. In some embodiments, the cell is a skeletal muscle cell. Non-limiting examples of skeletal muscles include the biceps, the triceps, the quadriceps, the tibialis interior, the gastrocnemius muscle, and diaphragm. In some embodiments, the method is conducted ex vivo. In some embodiments, the cell is a cell in an ex vivo sample that has been obtained from a subject.

In some embodiments, the payload comprises a transgene. In some embodiments, the transgene is a nucleic acid sequence heterologous to the vector sequences flanking the transgene which encodes a polypeptide, RNA (e.g., a miRNA or siRNA) or other product of interest. In embodiments, the nucleic acid of the transgene is operatively linked to a regulatory component in a manner sufficient to promote transgene transcription, translation, and/or expression in a host cell.

In aspects, a transgene is any polypeptide- or RNA-encoding sequence and the transgene selected will depend upon the use envisioned. In some embodiments, a transgene comprises a reporter sequence, which upon expression produces a detectable signal. Such reporter sequences include, without limitation, DNA sequences encoding colorimetric reporters (e.g., p-lactamase, p3-galactosidase (LacZ), alkaline phosphatase), cell division reporters (e.g., thymidine kinase), fluorescent or luminescence reporters (e.g., green fluorescent protein (GFP) or luciferase), resistance conveying sequences (e.g., chloramphenicol acetyltransferase (CAT)), or membrane bound proteins including to which high affinity antibodies directed thereto exist or can be produced by conventional means, e.g., comprising an antigen tag, e.g., hemagglutinin or Myc.

In some embodiments, a reporter sequence operably linked with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. In some embodiments, the transgene encodes a product which is useful in biology and medicine, such as RNA, proteins, peptides, enzymes, dominant negative mutants. In some embodiments, the RNA comprises a tRNA, ribosomal RNA, dsRNA, catalytic RNAs, small hairpin RNA, siRNA, trans-splicing RNA, and antisense RNAs. In some embodiments, the RNA inhibits or abolishes expression of a targeted nucleic acid sequence in a treated subject (e.g., a human or animal subject).

In some embodiments, the transgene is used to correct or ameliorate gene deficiencies. In some embodiments, gene deficiencies include deficiencies in which normal genes are expressed at less than normal levels or deficiencies in which the functional gene product is not expressed. In some embodiments, the transgene encodes a therapeutic protein or polypeptide which is expressed in a host cell. In some embodiments, a dependoparvovirus particle comprises or delivers multiple transgenes, e.g., to correct or ameliorate a gene defect caused by a multi-subunit protein. In some embodiments, a different transgene (e.g., each situated/delivered in a different dependoparvovirus particle, or in a single dependoparvovirus particle) is used to encode each subunit of a protein, or to encode different peptides or proteins, e.g., when the size of the DNA encoding the protein subunit is large, e.g., for immunoglobulin, platelet-derived growth factor, or dystrophin protein. In some embodiments, different subunits of a protein are encoded by the same transgene, e.g., a single transgene encoding each of the subunits with the DNA for each subunit separated by an internal ribozyme entry site (IRES). In some embodiments, the DNA is separated by sequences encoding a 2A peptide, which self-cleaves in a post-translational event. See, e.g., Donnelly et al, J. Gen. Virol., 78(Pt 1):13-21 (January 1997); Furler, et al, Gene Ther., 8(11):864-873 (June 2001); Klump et al., Gene Ther 8(10):811-817 (May 2001) (incorporated herein by reference in its entirety).

In some embodiments, virus particles comprising a genome are provided, wherein the genome includes a nucleic acid expression construct. The nucleic acid expression construct can include a heterologous transgene and one or more regulatory elements.

In some embodiments, the regulatory elements include a promotor, e.g., a promoter that is active in a target cell or tissue type of interest. In some embodiments, the promoter is a ubiquitous promoter. In other embodiments, the promoter is selective or specific to a target cell or tissue type (e.g., CNS and/or muscle (either broad muscle expression or skeletal muscle)), and, optionally, is less (or not) active in a cell or tissue type where transgene expression is not desired (e.g., liver, spleen, PNS, or any combination of two or all of the foregoing). A promoter that is selective for a first cell or tissue type over a second cell or tissue type is active in the first cell or tissue type and less active or silent in the second cell or tissue type. In some embodiments, the promoter is a CNS-specific or CNS-selective promoter. In some embodiments, the promoter is a muscle-specific promoter-specific or muscle-selective promoter. In some embodiments, the promoter is active in both CNS and muscle tissue.

In some embodiments, the promoter is a ubiquitous or constitutive promoter active in a mammalian cell, for example a human cell, for example, in a human cell type of interest. In some embodiments, the cell type is a CNS cell such as, for example, a neuronal cell, a glial cell, an endothelia cell, and the like. In some embodiments, the cell type is a skeletal muscle cell. Examples of ubiquitous promoters include, but are not limited, to a CAG promoter (hybrid from a cytomegalovirus early enhancer element, a chicken-beta actin promoter, e.g., the first exon and the first intron of the chicken beta actin gene, and the splice acceptor of the rabbit beta globin gene), chicken-beta actin promoter, CBA promoter, CBh promoter, CB6 promoter, CMV promoter, human EF1-alpha promoter, PGK promoter, ubiquitin C (UBC) promoter and fragments thereof. In some embodiments, the promoter is a tissue-specific promoter, for example, a promoter specific in CNS tissue or cells of the CNS. Examples of CNS-specific promoters include but are not limited to a synapsin (SYN or SYN1) promoter, a neuron-specific enolase (NSE) promoter, a Ca²⁺/calmodulin-dependent kinase subunit a (CaMKII) promoter, a synapsin I with a minimal CMV sequence (Synl-minCMV) promoter, a glial fibrillary acidic protein (GFAP) promoter, a internexin neuronal intermediate filament protein alpha (INA) promoter, a nestin (NES) promoter, a neurofilament light chain (NfL) promoter, a neurofilament heavy chain (NfH) promoter, a myelin-associated oligodendrocyte basic protein (MOBP) promoter, a myelin basic protein (MBP) promoter, a tyrosine hydroxylase (TH) promoter, a forkhead box A2 (FOXA2) promoter, a aldehyde dehydrogenase 1 family member L1 (ALDH1L1) promoter, a glutamate decarboxylase 2 (GAD2) promoter, a riken gene A930098C07Rik (A93) promoter, a somatostatin (SST) promoter, a platelet derived growth factor receptor alpha (PDGFRA) promoter, a glutamate receptor metabotropic 1 (GRM1) promoter, a C-type natriuretic peptide precursor (NPPC) promoter, a adrenomedullin (ADM) promoter, a type 2 lactosamine alpha-2,3-sialyltransferase (ST3GAL6) promoter, a ras responsive element binding protein 1 (RREB1) promoter, a deiodinase iodothyronine type II (D102) promoter, an excitatory amino acid transporter 2 (EAAT2) promoter, a nuclear receptor subfamily 2 group F member 2 (NR2F2) promoter, a platelet-derived growth factor (PDGF) promoter, a methyl-CpG binding protein 2 (MeCP2) promoter, and mouse, primate or human homologs of any of the forgoing, and fragments (e.g., active fragments) of any of the foregoing. In embodiments, the CNS-specific promoter is a neuron specific promoter. In embodiments, the CNS-specific promoter is an astrocyte-specific promoter. In some embodiments, the promoter is a promoter specific in muscle tissue or muscle cells, e.g., a desmin, MCK, TNNT2, or smooth muscle 22 (SM22) promoter. Further exemplary muscle specific promoters are described below.

In some embodiments, the promoter is a CBh promoter. An exemplary CBh promoter sequence is set forth as SEQ ID NO:253. In some embodiments, the CBh promoter comprises a nucleotide sequence having at least 90%, at least 95%, at least 96%, at least 97%, or at least 98%, at least 99%, or 100% sequence identity to SEQ ID NO:253

In other embodiments, the promoter is a synapsin promoter, for example a human synapsin promoter (hSYN). An exemplary hSYN promoter sequence is set forth as SEQ ID NO:254. In some embodiments, the CBh promoter comprises a nucleotide sequence having at least 90%, at least 95%, at least 96%, at least 97%, or at least 98%, at least 99%, or 100% sequence identity to SEQ ID NO:254.

In some embodiments, the nucleic acid expression construct comprises an intron. In embodiments, the intron is disposed between the promoter and the heterologous transgene. In some aspects, the intron is disposed 5′ to the heterologous transgene on the expression construct, for example immediately 5′ to the heterologous transgene or 100 nucleotides or less 5′ to the heterologous transgene. In some aspects, the intron is a chimeric intron derived from human b-globin and Ig heavy chain (also known as b-globin splice donor/immunoglobulin heavy chain splice acceptor intron, or b-globin/IgG chimeric intron; Reed, R., et al. Genes and Development, 1989, incorporated herein by reference in its entirety). In other aspects, the intron is a VH4 intron or a SV40 intron.

As provided herein, in some embodiments, virus particles comprising a payload, wherein the payload includes a nucleic acid that includes a heterologous transgene are provided. In some embodiments, the heterologous transgene encodes an RNA interference agent, for example a siRNA, shRNA or other interfering nucleic acid.

In some embodiments, the payload includes a heterologous transgene that encodes a therapeutic polypeptide. In some aspects, the heterologous transgene is a human gene or fragment thereof. In some aspects, the therapeutic polypeptide is a human protein. In some embodiments, the heterologous transgene of the virus particle encodes a molecule useful in treating a disease, and the virus particle is administered to a patient in need thereof to treat said disease. In some aspects the payload comprises a molecule that is effective in treating chronic CNS disease, such as, for example, an RNA interference nucleotide (e.g., shRNA, siRNA or miRNA that inhibits APOL-1). Examples of diseases (and heterologous transgenes or molecules encoded by said heterologous transgenes) according to the present disclosure include: MPSI (alpha-L-iduronidase (IDUA)); MPS II—Hunter syndrome (iduronate-2-sulfatase (IDS)); Ceroid lipofuscinosis-Batten disease (CLN1, CLN2, CLN10, CLN13, CLN5, CLN11, CLN4, CNL14, CLN3, CLN6, CLN7, CLN8, CLN12); MPS IIIa—Sanfilippo Type A syndrome (heparin sulfate sulfatase (also called N-sulfoglucosamine sulfohydrolase (SGSH)); MPS IIIB—Sanfilippo Type b syndrome (N-acetyl-alpha-D-glucosaminidase (NAGLU)); MPS VI—Maroteaux-Lamy syndrome (arylsulfatase B); MPS IV A—Morquio syndrome type A (GALNS); MPS IV B—Morquio syndrome type B (GLB1); chronic or neuropathic pain; Osteogenesis Imperfecgta Type I, II, III or IV (COL1AI and/or COL1A2); hereditary angioedema (SERPING1, C1NH); Osteogenesis Imperfecta Type V (IFITM5); Osteogenesis Imperfecta Type VI (SERPINF1); Osteogenesis Imperfecta Type VII (CRTAP); Osteogenesis Imperfecta Type VIII (LEPRE1 and/or P3H1); Osteogenesis Imperfecta Type IX (PPIB); Gaucher disease type I, II and III (Glucocerebrosidase; GBAI); Parkinson's Disease (Glucocerebrosidase; GBAI and/or dopamine decarboxylase); Pompe (acid maltase; GAA; hGAA); Metachromatic leukodystrophy (Aryl sulfatase A); MPS VII—Sly syndrome (beta-glucuronidase); MPS VIII (glucosamine-6-sulfate sulfatase); MPS IX (Hyaluronidase); maple syrup urine disease (BCKDHA, BCKDHB, and/or DBT); Niemann-Pick disease (Sphingomyelinase); Parkinson's disease (anti-alpha synuclein RNAi); Alzheimer's disease (anti-mutant APP RNAi); Niemann-Pick disease without sphingomyelinase deficiency (NPC1 or NPC gene encoding a cholesterol metabolizing enzyme); Tay-Sachs disease (alpha subunit of beta-hexosaminidase); Sandhoff disease (both alpha and beta subunit of beta-hexosaminidase), Fabry Disease (alpha-galactosidase), Fucosidosis (fucosidase (FUCAI)), Alpha-mannosidosis (alpha-mannosidase); Beta-mannosidosis (beta-mannosidase); Wolman disease (cholesterol ester hydrolase); Dravet syndrome (SCN1A, SCN1B, SCN2A, GABRG2); Parkinson's disease (Neurturin); Parkinson's disease (glial derived growth factor (GDGF)); Parkinson's disease (tyrosine hydroxylase); frontotemporal dementia (progranulin); Angleman syndrome (ubiquitin protein ligase 3A (UBE3A), gene editing systems targeting a UBE3A inhibitory RNA (UBE3A-anitsense transcript)); Parkinson's disease (glutamic acid decarboxylase; FGF-2; BDGF); Spinal Muscular Atrophy (SMN, including SMN1 or SMN2); Friedreich's ataxia (Frataxin); Amyotrophic lateral sclerosis (ALS) (SOD1 inhibitor, e.g., anti-SOD1 RNAi); Glycogen Storage Disease la (Glucose-6-phosphatase); XLMTM (MTMI); Crigler Najjar (UGTIAI); CPVT (CASQ2); spinocerebellar ataxia (ATXN2; ATXN3 or other ATXN gene; anti-mutant Machado-Joseph disease/SCA3 allele RNAi); Rett syndrome (MECP2 or fragment thereof); Achromatopsia (CNGB3, CNGA3, GNAT2, PDE6C); Choroidermia (CDM); Danon Disease (LAMP2); Cystic Fibrosis (CFTR or fragment thereof); Duchenne Muscular Dystrophy (Mini-/Micro-Dystrophin Gene); SARS-Cov-2 infection (anti-SARS-Cov-2 RNAi, SARS-Cov-2 genome fragments or S protein (including variants)); Limb Girdle Muscular Dystrophy Type 2C—Gamma-sarcoglycanopathy (human-alpha-sarcoglycan); Advanced Heart Failure (SERCA2a); Rheumatoid Arthritis (TNFR:Fc Fusion; anti-TNF antibody or fragment thereof); Leber Congenital Amaurosis (GAA); X-linked adrenoleukodystrophy (ABCD1); Limb Girdle Muscular Dystrophy Type 2C—Gamma-sarcoglycanopathy (gamma-sarcoglycan); Angelman syndrome (UBE3A); Retinitis Pigmentosa (hMERTK); Age-Related Macular Degeneration (sFLTO1); Phelan-McDermid syndrome (SHANK3; 22q13.3 replacement); Becker Muscular Dystrophy and Sporadic Inclusion Body Myositis (huFollistatin344); Parkinson's Disease (GDNF); Metachromatic Leukodystrophy —MLD (cuARSA); Hepatitis C (anti-HCV RNAi); Limb Girdle Muscular Dystrophy Type 2D (hSGCA); Human Immunodeficiency Virus Infections; (PG9DP); Acute Intermittant Porphyria (PBGD); Leber's Hereditary Optical Neuropathy (PIND4v2); Alpha-1 Antitrypsin Deficiency (alphalAT); X-linked Retinoschisis (RS1); Choroideremia (hCHM); Giant Axonal Neuropathy (GAN); Hemophilia B (Factor IX); Homozygous FH (hLDLR); Dysferlinopathies (DYSF); Achromatopsia (CNGA3 or CNGB3); Progressive supranuclear palsy (MAPT; anti-Tau; anti-MAPT RNAi); Omithine Transcarbamylase deficiency (OTC); Hemophilia A (Factor VIII); Age-related macular degeneration (AMD), including wetAMD (anti-VEGF antibody or RNAi); X-Linked Retinitis Pigmentosa (RPGR); Myotonic dystrophy Type 1 (DMPK; anti-DMPK RNAi, including anti-CTG trinucleotide repeat RNAi); Myotonic dystrophy Type 2 (CNBP); Facioscapulohumeral muscular dystrophy (D4Z4 DNA); oculopharynggeal muscular dystrophy (PABPN1; mutated PABPN1 inhibitor (e.g., RNAi)); Mucopolysaccharidosis Type VI (hARSB); Leber Hereditary Optic Neuropathy (ND4); X-Linked myotubular Myopathy (MTMI); Crigler-Najjar Syndrome (UGTIAI); Retinitis Pigmentosa (hPDE6B); Mucopolysaccharidosis Type 3B (hNAGLU); Duchenne Muscular Dystrophy (GALGT2); Alzheimer's Disease (NGF; ApoE4, ApoE2; ApoE3; Anti-ApoE RNAi, MAPT, anti-Tau antibody, anti-amyloid beta antibody (e.g., aducanumab)); multiple system atrophy; Familial Lipoprotein Lipase Deficiency (LPL); Alpha-1 Antitrypsin Deficiency (hAAT); Leber Congenital Amaurosis 2 (hRPE65v2); Batten Disease; Late Infantile Neuronal Lipofuscinosis (CLN2); Huntington's disease (HTT; anti-HTT RNAi); Fragile X syndrome (FMR1); Leber's Hereditary Optical Neuropathy (PIND4v2); Aromatic Amino Acid Decarboxylase Deficiency (hAADC); Retinitis Pigmentosa (hMERKTK); and Retinitis Pigmentosa (RLBPI). In some aspects, the payload comprises a molecule that is effective in treating muscle-related disease. Exemplary, non-limiting muscle-related diseases are described below.

In some embodiments, the heterologous transgene encodes a therapeutic polypeptide. In some aspects, the heterologous transgene is a human gene or fragment thereof. In some aspects, the therapeutic polypeptide is a human protein. In some aspects, the heterologous transgene encodes an antibody or fragment thereof (for example an antibody light chain, an antibody heavy chain, a Fab or an scFv). Examples of antibodies or fragments thereof that are encoded by the heterologous transgene include but are not limited to; and an anti-Ab antibody (e.g., solanezumab, GSK933776, and lecanemab), anti-sortilin (e.g., AL-001), anti-Tau (e.g., ABBV-8E12, UCB-0107, and NI-105), anti-SEMA4D (e.g., VX15/2503), anti-alpha synuclein (e.g., prasinezumab, NI-202, and MED-1341), anti-SOD1 (e.g, NI-204), anti-CGRP receptor (e.g., eptinezumab, fremanezumab, or galcanezumab), anti-VEGF (e.g., sevacizumab, ranibizumab, bevacizumab, and brolucizumab), anti-EpoR (e.g., LKA-651,), anti-ALKI (e.g., ascrinvacumab), anti-C5 (e.g., tesidolumab, ravulizumab, and eculizumab), anti-CD105 (e.g., carotuximab), anti-CCIQ (e.g., ANX-007), anti-TNFa (e.g., adalimumab, infliximab, and golimumab), anti-RGMa (e.g., elezanumab), anti-TTR (e.g., NI-301 and PRX-004), anti-CTGF (e.g., pamrevlumab), anti-IL6R (e.g., satralizumab, tocilizumab, and sarilumab), anti-IL6 (e.g., siltuximab, clazakizumab, sirukumab, olokizumab, and gerilimzumab), anti-IL4R (e.g., dupilumab), anti-IL17A (e.g., ixekizumab and secukinumab), anti-IL5R (e.g., reslizumab), anti-IL-5 (e.g., benralizumab and mepolizumab), anti-IL13 (e.g., tralokinumab), anti-IL12/IL23 (e.g., ustekinumab), anti-CD 19 (e.g., inebilizumab), anti-IL31RA (e.g., nemolizumab), anti-ITGF7 mAb (e.g., etrolizumab), anti-SOST mAb (e.g., romosozumab), anti-IgE (e.g., omalizumab), anti-TSLP (e.g., nemolizumab), anti-pKal mAb (e.g., Ianadelumab), anti-ITGA4 (e.g., natalizumab), anti-ITGA4B7 (e.g., vedolizumab), anti-BLyS (e.g., belimumab), anti-PD-1 (e.g., nivolumab and pembrolizumab), anti-RANKL (e.g., denosumab), anti-PCSK9 (e.g., alirocumab and evolocumab), anti-ANGPTL3 (e.g., evinacumab*), anti-OxPL (e.g., E06), anti-fD (e.g., lampalizumab), or anti-MMP9 (e.g., andecaliximab), optionally wherein the heavy chain (Fab and Fc region) and the light chain are separated by a self-cleaving furin (F)/F2A or furin (F)/T2A, IRES site, or flexible linker, for example, ensuring expression of equal amounts of the heavy and the light chain polypeptides.

In some embodiments, the virus particle comprises a heterologous transgene encoding a genome editing system. Examples include a CRISPR genome editing system (e.g., one or more components of a CRISPR genome editing system such as, for example, a guide RNA molecule and/or an RNA-guided nuclease such as a Cas enzyme such as Cas9, Cpf1 and the like), a zinc finger nuclease genome editing system, a TALEN genome editing system or a meganuclease genome editing system. In embodiments, the genome editing system targets a mammalian, e.g., human, genomic target sequence. In embodiments, the virus particle includes a heterologous transgene encoding a targetable transcription regulator. Examples include a CRISPR-based transcription regulator (for example, one or more components of a CRISPR-based transcription regulator, for example, a guide RNA molecule and/or an enzymatically-inactive RNA-guided nuclease/transcription factor (“TF”) fusion protein such as a dCas9-TF fusion, dCpf1-TF fusion and the like), a zinc finger transcription factor fusion protein, a TALEN transcription regulator or a meganuclease transcription regulator.

In some embodiments, components of a therapeutic molecule or system are delivered by more than one unique virus particle (e.g., a population that includes more than one unique virus particles). In other embodiments, the therapeutic molecule or components of a therapeutic molecule or system are delivered by a single unique virus particle (e.g., a population that includes a single unique virus particle).

In embodiments, the transgene encodes any biologically active product or other product, e.g., a product desirable for study. Suitable transgenes may be readily selected by persons of skill in the art, such as those, but not limited to, those described herein.

Other examples of proteins encoded for by the transgene include, but are not limited to, colony stimulating factors (CSF); blood factors, such as p-globin, hemoglobin, tissue plasminogen activator, and coagulation factors; interleukins; soluble receptors, such as soluble TNF-α receptors, soluble VEGF receptors, soluble interleukin receptors (e.g., soluble IL-1 receptors and soluble type II IL-1 receptors), or ligand-binding fragments of a soluble receptor; growth factors, such as keratinocyte growth factor (KGF), stem cell factor (SCF), or fibroblast growth factor (FGF, such as basic FGF and acidic FGF); enzymes; chemokines, enzyme activators, such as tissue plasminogen activator; angiogenic agents, such as vascular endothelial growth factors, glioma-derived growth factor, angiogenin, or angiogenin-2; anti-angiogenic agents, such as a soluble VEGF receptor; a protein vaccine; neuroactive peptides, such as nerve growth factor (NGF) or oxytocin; thrombolytic agents; tissue factors; macrophage activating factors; tissue inhibitors of metalloproteinases; or IL-1 receptor antagonists.

The disclosure is further directed, in part, to a method of delivering a payload to a subject, e.g., an animal or human subject. In some embodiments, a method of delivering a payload to a subject comprises administering to the subject a dependoparvovirus particle comprising a variant polypeptide (e.g., described herein) comprising the payload, e.g., in a quantity and for a time sufficient to deliver the payload. In some embodiments, the dependoparvovirus particle is a dependoparvovirus particle described herein and comprises a payload described herein. In some embodiments, the particle delivers the payload to the CNS. In some embodiments, the delivery to the CNS is increased as compared to a particle without the variant capsid polypeptide or as compared to a wild-type capsid polypeptide, e.g., a particle with capsid polypeptides of SEQ ID NO:1. In some embodiments, the particle delivers the payload to skeletal muscle tissue. In some embodiments, the delivery to the skeletal muscle tissue is increased as compared to a particle without the variant capsid polypeptide or as compared to a wild-type capsid polypeptide, e.g., a particle with capsid polypeptides of SEQ ID NO:1.

In some embodiments, virus particles comprising a genome are provided, wherein the genome includes a nucleic acid expression construct including a heterologous transgene and one or more regulatory elements, where the one or more regulatory elements include a muscle (e.g., skeletal muscle) specific promoter. Examples of muscle specific promoters that can be used to drive expression of a transgene in skeletal muscle include but are not limited to Desmin (DES), CAMK, Mb, myosin (e.g., myo-3), dystrophin, muscle creatine kinase (MCK), MHCK7, CK6, CK7, CK8, CK8e, dMCK, tMCK, MH, SPc-5-12 (also known as C5-12), alpha skeletal actin (ASKA), SP-301, E-syn, myosin light chain (MLC), myosin heavy chain (MHC), four and a half LIM domains protein 1 (FHL1), alpha 2 actinin (ACTN2), filamin-C(FLNC), sarcoplasmic/endoplasmic reticulum calcium ATPase 1 (ATP2A1), troponin I type 1 (TNN11), myosin-1 (MYH1), phosphorylatable, fast skeletal muscle myosin light chain (MYLPF), alpha-3 chain tropomyosin (TPM3), Pitx3, and ankyrin repeat domain-containing protein 2 (ANKRD2) promoters. Promoters capable of driving expression in skeletal muscle are further described in Skopenkova et al., 2021, Acta Naturae 13(1):47-58, Piekarowicz et al., 2019, Mol Ther Methods Clin Dev. 15:157-169, Wang, 2008 Gene Ther. 15:1489-1499, Coulon et al., 2007, JBC 282(45):33192-33200, WO 2021/127655, and WO 2023/006890, the contents of each of which are incorporated herein by reference in their entireties.

In some aspects, the payload comprises a molecule that is effective in treating a muscle disease, such as, for example, a protein or an RNA interference nucleotide (e.g., shRNA, siRNA or miRNA).

Exemplary muscle tissue related diseases that can be treated include but are not limited to Acid Maltase Deficiency (AMD), Amyotrophic Lateral Sclerosis (ALS), Andersen-Tawil Syndrome, Barth syndrome (TAZ), Becker Muscular Dystrophy (BMD), Becker Myotonia Congenita, Bethlem Myopathy, Bulbospinal Muscular Atrophy (Spinal-Bulbar Muscular Atrophy), Carnitine Deficiency, Carnitine Palmityl Transferase Deficiency (CPT Deficiency), Central Core Disease (CCD), Centronuclear Myopathy, Charcot-Marie-Tooth Disease (CMT), Congenital Muscular Dystrophy (CMD), Congenital Myasthenic Syndromes (CMS), Congenital Myotonic Dystrophy, Cori Disease (Debrancher Enzyme Deficiency), Danon disease, Debrancher Enzyme Deficiency, Dejerine-Sottas Disease (DSD), Dermatomyositis (DM), Distal Muscular Dystrophy (DD), Distal myopathy with anterior tibial onset, Duchenne Muscular Dystrophy (DMD), Dystrophia Myotonica (Myotonic Muscular Dystrophy), Emery-Dreifuss Muscular Dystrophy (EDMD), Endocrine Myopathies, Eulenberg Disease (Paramyotonia Congenita), Facioscapulohumeral Muscular Dystrophy (FSH or FSHD), Finnish (Tibial) Distal Myopathy, Forbes Disease (Debrancher Enzyme Deficiency), Friedreich's Ataxia (FA), Fukuyama Congenital Muscular Dystrophy, Glycogenosis Type 10, Glycogenosis Type 11, Glycogenosis Type 2, Glycogenosis Type 3, Glycogenosis Type 5, Glycogenosis Type 7, Glycogenosis Type 9, Gowers-Laing Distal Myopathy, Hauptmann-Thanheuser MD (Emery-Dreifuss Muscular Dystrophy), Hereditary Inclusion-Body Myositis, Hereditary Motor and Sensory Neuropathy (Charcot-Marie-Tooth Disease), Hyperthyroid Myopathy, Hypothyroid Myopathy, Inclusion-Body Myositis (IBM), Inherited Myopathies, Integrin-Deficient Congenital Muscular Dystrophy, Kennedy Disease (Spinal-Bulbar Muscular Atrophy), Kugelberg-Welander Disease (Spinal Muscular Atrophy), Lactate Dehydrogenase Deficiency, Lambert-Eaton Myasthenic Syndrome (LEMS), Limb-Girdle Muscular Dystrophy (LGMD), Lou Gehrig's Disease (Amyotrophic Lateral Sclerosis), McArdle Disease (Phosphorylase Deficiency), Merosin-Deficient Congenital Muscular Dystrophy, Metabolic Diseases of Muscle, Mitochondrial Myopathy, Miyoshi myopathy, Miyoshi Distal Myopathy, Motor Neurone Disease, Muscle-Eye-Brain Disease, Myasthenia Gravis (MG), Myoadenylate Deaminase Deficiency, Myofibrillar Myopathy, Myophosphorylase Deficiency, Myotonia Congenita (MC), Myotonic Muscular Dystrophy (MMD), Myotubular Myopathy (MTM or MM), Nemaline Myopathy, Nonaka Distal Myopathy, Oculopharyngeal Muscular Dystrophy (OPMD), Paramyotonia Congenita, Pearson Syndrome, Periodic Paralysis, Peroneal Muscular Atrophy (Charcot-Marie-Tooth Disease), Phosphofructokinase Deficiency, Phosphoglycerate Kinase Deficiency, Phosphoglycerate Mutase Deficiency, Phosphorylase Deficiency, Phosphorylase Deficiency, Polymyositis (PM), Pompe Disease (Acid Maltase Deficiency), Primary merosin deficiency (LAMA2), Progressive External Ophthalmoplegia (PEO), Rod Body Disease (Nemaline Myopathy), Spinal Muscular Atrophy (SMA), Spinal-Bulbar Muscular Atrophy (SBMA), Steinert Disease (Myotonic Muscular Dystrophy), Tarui Disease (Phosphofructokinase Deficiency), Thomsen Disease (Myotonia Congenita), Ullrich Congenital Muscular Dystrophy, Walker-Warburg Syndrome (Congenital Muscular Dystrophy), Welander Distal Myopathy, Werdnig-Hoffmann Disease (Spinal Muscular Atrophy), and ZASP-Related Myopathy.

Payloads suitable for treating muscle-related disease are known in the art and include the following disease (suitable payload) combinations: Barth syndrome (TAZ), Primary merosin deficiency (LAMA2), Duchenne muscular dystrophy (DMD), Becker muscular dystrophy (BMD), Danon disease (LAMP2), Limb girdle muscular dystrophy (Subtypes and affected genes: LGMD1A (TTID), LGMD1B (LMNA), LGMD1C (CAV3), LGMD1D (DNAJB6), LGMD1E (DES), LGMD1F (TNPO3), LGMD1G (HNRPDL), LGMD1H, LGMD2A (CAPN3), LGMD2B (DYSF), LGMD2C (SGCG), LGMD2D (SGCA), LGMD2E (SGCB), LGMD2F (SGCD), LGMD2G (TCAP), LGMD2H (TRIM32), LGMD2I (FKRP), LGMD2J (TTN), LGMD2K (POMT1), LGMD2L (AN05), LGMD2M (FKTN), LGMD2N (POMT2), LGMD20 (POMGNT1), LGMD2Q (PLEC1)), Miyoshi myopathy (DYSF), Distal myopathy with anterior tibial onset (DYSF), Welander distal myopathy (TIA1), Gowers-Laing distal myopathy (MYH7), Facioscapulohumeral muscular dystrophy (Subtypes and affected genes: Type 1 (DUX4), Type 2 (SMCHD1)), Oculopharyngeal muscular dystrophy (PABPN1), myotonic dystrophy (Subtypes and affected genes: DM1 (DMPK) and DM2 (ZNF9)), congenital myotonia (CLCN1), paramyotonia congenital (SCN4A), myotubular myopathy (MTM1), glycogen storage disease type II (Pompe disease) (GAA).

In some embodiments, the payload is selected from: TAZ, LAMA2, DMD, LAMP2, CAPN3, DYSF, SGCA, SGCB, FKRP, PABPN1, MTM1, and GAA.

Further exemplary diseases that can be treated and further exemplary heterologous transgenes that can be delivered via the viral particles of the disclosure are provided in Table 2, Table 3, and Table 4.

TABLE 2
Exemplary CNS Indications and Transgenes

| | | UniProt |
|---|---|---|
| | | Accession # |
| Indication | Transgene | (Human) |
| Achromatopsia - color blindness | Cyclic nucleotide-gated cation channel | Q16281 |
| | alpha-3 (CNGA3) | |
| | Cyclic nucleotide-gated cation channel | Q9NQW8 |
| | beta-3 (CNGB3) | |
| | Guanine nucleotide-binding protein G(t) | P19087 |
| | subunit alpha-2 (GNAT2) | |
| | Cone cGMP-specific 3′,5′-cyclic | P51160 |
| | phosphodiesterase subunit alpha | |
| | (PDE6C) | |
| Acute Intermittent Porphyria | Porphobilinogen deaminase (PBGD), | P08397 |
| | HMBS | |
| Adie syndrome - Adie's pupil | MPZ | P25189 |
| Age-Related Macular Degeneration | Vascular endothelial growth factor | P17948 |
| | receptor 1 (FLT1) | |
| | Vascular endothelial growth factor A | P15692 |
| | (VEGFA) | |
| Agenesis of the Corpus Callosum | SLC12A6 | Q9UHW9 |
| (ACCPN) | | |
| Aicardi-Goutieres syndrome | TREX1 | Q9NSU2 |
| Alexander disease | GFAP | P14136 |
| Alpers syndrome | POLG | P54098 |
| Alternating hemiplegia | ATP1A2 | P50993 |
| | ATP1A3 | P13637 |
| Alzheimer's disease | NGF | P01138 |
| | ApoE | P02649 |
| | Presenilin (PSEN1) | A0A024R6A3 |
| | Presenilin-2 (PSEN2) | P49810 |
| | Amyloid-beta precursor protein (APP) | P05067 |
| | ADAM10 | O14672 |
| | MAPT, Tau | P10636 |
| Amyotrophic lateral sclerosis (ALS) - | Superoxide dismutase-1 (SOD1) | P00441 |
| Lou Gehrig's disease | | |
| Amyotrophy, hereditary neuralgic | SEPT9 | Q9UHD8 |
| Angleman syndrome | Ubiquitin-protein ligase E3A (UBE3A) | Q05086 |
| Aromatic L-amino acid decarboxylase | DDC | P20711 |
| deficiency (AADCD) | | |
| Ataxia | APTX | Q7Z2E3 |
| | KCNA1 | Q09470 |
| | CACNA1A | O00555 |
| Ataxia Telangiectasia - Louis-Bar | Serine-protein kinase ATM (ATM) | Q13315 |
| syndrome | | |
| Attention deficit hyperactivity disorder | DRD4 | P21917 |
| (ADHD) | CDH2 | P19022 |
| Becker muscular dystrophy | Follistatin (FST) | P19883 |
| | DMD | P11532 |
| Benign essential blepharospasm | DRD5 | P21918 |
| Bradbury-Eggleston syndrome - pure | COQ2 | Q96H96 |
| autonomic failure | | |
| Bulbar palsy (BVVLS1) | SLC52A3 | Q9NQ40 |
| Canavan disease - aminoacylase 2 | ASPA | P45381 |
| deficiency | | |
| Carpal tunnel syndrome | TTR | P02766 |
| Cavernoma - Cavernous angioma - | KRIT1 | O00522 |
| Cavernous malformations | | |
| Cerebellar Hypoplasia (CHEGDD) | OXR1 | Q8N573 |
| Cerebellar ataxia (CAMRQ2) | WDR81 | Q562E7 |
| Cerebral Arteriopathy with SCI and | NOTCH3 | Q9UM47 |
| Leukoencephalopathy (CADASIL) | | |
| Cerebral gigantism - Sotos syndrome | NSD1 | Q96L73 |
| 1 | | |
| Cerebro-oculo-facio-skeletal syndrome | ERCC6 | Q03468 |
| (COFS) | | |
| Ceroid lipofuscinosis - Batten disease | CLN1 (PPT1) | P50897 |
| | CLN2 (TPP1) | O14773 |
| | CLN3 (battenin) | Q13286 |
| | CLN4 | Q9H3Z4 |
| | CLN5 | O75503 |
| | CLN6 | Q9NWW5 |
| | CLN7 (MFSD8) | Q8NHS3 |
| | CLN8 | Q9UBY8 |
| | CLN10 (cathepsin D) | P07339 |
| | CLN11 (progranulin) | P28799 |
| | CLN12 (ATP13A2) | Q9NQ11 |
| | CLN13 (cathepsin F) | Q9UBX1 |
| | CLN14 (KCTD7) | Q96MP8 |
| Charcot-Marie-Tooth disease | PMP22 | Q01453 |
| | MPZ | P25189 |
| | DNM2 | P50570 |
| | MFN2 | O95140 |
| | KIF1B | O60333 |
| | SBF2 | Q86WG5 |
| | PNKP | Q96T60 |
| | GDAP1 | Q8TB36 |
| | LMNA | P02545 |
| | FGD4 | Q96M96 |
| | MTMR2 | Q13614 |
| Chorea | NKX2-1 | P43699 |
| Choreoacanthocytosis | VPS13A | Q96RL7 |
| Choroideremia | Rab escort protein (Rep1), CHM | P24386 |
| Chronic Inflammatory Demyelinating | PMP22 | Q01453 |
| Polyneuropathy (CIDP) | | |
| Cockayne syndrome B (CSB) | ERCC6 | Q03468 |
| Coffin-Lowry syndrome | RPS6KA3 | P51812 |
| Craniosynostosis | MSX2 | P35548 |
| | TWIST1 | Q15672 |
| | SKI | P12755 |
| | SMAD6 | O43541 |
| Creutzfeldt-Jakob disease | PRNP | P04156 |
| | HLA-DQB1 | P01920 |
| Crigler-Najjar Syndrome - | UDP-glucuronosyltransferase 1A1 | P22309 |
| hyperbilirubinemia | (UGT1A1) | |
| Cushing Syndrome | PRKACA | P17612 |
| Dentatorubral atrophy (DRPLA) | ATN1 | P54259 |
| Developmental and Epileptic | ARX | Q96QS3 |
| Encephalopathy | FGF12 | P61328 |
| | PIGP | P57054 |
| | GABRB3 | P28472 |
| | NECAP1 | Q8NC96 |
| Developmental Dyspraxia - speech- | FOXP2 | O15409 |
| language disorder 1 (SPCH1) | | |
| Dravet syndrome | Sodium channel protein type 1 subunit | P35498 |
| | alpha (SCN1A) | |
| | SCN1B | Q07699 |
| | SCN2A | Q99250 |
| | GABA receptor subunit gamma-2 | P18507 |
| | (GABRG2) | |
| Dysautonomia - -Day | ELP1 | O95163 |
| syndrome | | |
| Dystonias | GCH1 | P30793 |
| | TOR1A | O14656 |
| | SGCE | O43556 |
| | TUBB4A | P04350 |
| Encephalocele | COL18A1 | P39060 |
| Epilepsy disorders | GRIN2A | Q12879 |
| | CSTB | P04080 |
| | STARD7 | Q9NQZ5 |
| | DEPDC5 | O75140 |
| | PCDH19 | Q8TAB3 |
| Essential tremor | DRD3 | P35462 |
| | NOTCH2NLC | P0DPK4 |
| | FUS | P35637 |
| Fabry disease | alpha-galactosidase A (GLA) | P06280 |
| Farber disease - ceramidase | ASAH1 | Q13510 |
| deficiency | | |
| Fahr disease | SLC20A2 | Q08357 |
| Febrile Seizures | GABRG2 | P18507 |
| | ADGRV1 | Q8WXG9 |
| | CPA6 | P11509 |
| | SCN1A | P35498 |
| Friedreich's ataxia | Frataxin (FXN) | Q16595 |
| Frontotemporal dementia | Progranulin (GRN) | P28799 |
| | MAPT (tau) | P10636 |
| | PSEN1 | A0A024R6A3 |
| Fucosidosis | alpha-L-fucosidase (FUCA1) | P04066 |
| Fundus albipunctatus | RLBP1 | P12271 |
| Gaucher disease, types I, II and III | Glucocerebrosidase (GBA1) | P04062 |
| Generalized gangliosidoses (GM1, | GLB1 | P16278 |
| GM2, GM3) | | |
| Gerstmann-Straussler-Scheinker | PRNP | P04156 |
| disease | | |
| Giant axonal neuropathy | Gigaxonin (GAN) | Q9H2C0 |
| Glycogen storage disease II - Pompe | Acid maltase, lysosomal alpha- | P10253 |
| disease - Acid Maltase Deficiency | glucosidase (LYAG, GAA) | |
| Guillain-Barre syndrome | PMP22 | Q01453 |
| Hallervorden-Spatz disease - PKAN - | PANK2 | Q9BZ23 |
| NBIA1 | | |
| Hemiplegia Alterans | ATP1A2 | P50993 |
| | ATP1A3 | P13637 |
| Hereditary Neuropathies | WNK1 | Q9H4A3 |
| | MFN2 | O95140 |
| | HK1 | P19367 |
| | TFG | Q92734 |
| | SPTLC1 | O15269 |
| Heredopathia Atactica | PHYH | O14832 |
| Polyneuritiformis - Refsum disease | | |
| Holoprosencephalies | GLI2 | P10070 |
| | TGIF1 | Q15583 |
| | ZIC2 | O95409 |
| | PTCH1 | Q13635 |
| | SHH | Q15465 |
| Huntington's disease | HTT | P42858 |
| Hydrocephalus disorders | CCDC88C | Q9P219 |
| | WDR81 | Q562E7 |
| | TRIM71 | Q2Q1W2 |
| | MPDZ | O75970 |
| Incontinentia Pigmenti | IKBKG | Q9Y6K9 |
| Infantile Hypotonia | NALCN | Q8IZF0 |
| | TBCK | Q8TEA7 |
| | CCDC174 | Q6PII3 |
| | UNC80 | Q8N2C7 |
| Infantile Neuroaxonal Dystrophy | PLA2G6 | O60733 |
| Infantile Phytanic Acid Storage | PEX1 | O43933 |
| Disease (PBD1B) | | |
| Joubert Syndrome | INPP5E | Q9NRR6 |
| Kennedy Disease | Androgen receptor (AR) | P10275 |
| Klippel-Feil Syndrome | GDF6 | Q6KF10 |
| Krabbe disease - GALC deficiency | GALC | P54803 |
| Lambert-Eaton Myasthenic Syndrome | CACNA1A | O00555 |
| | CACNB2 | Q13936 |
| Landau-Kleffner Syndrome | GRIN2A | Q12879 |
| Late infantile neuronal lipofuscinosis | TPP1 | O14773 |
| (CLN2) | | |
| Lesch-Nyhan Syndrome | HPRT1 | P00492 |
| Leber congenital amaurosis - retinal | Retinal guanylyl cyclase 1 (GUCY2D) | Q02846 |
| blindness | Retinoid isomerohydrolase (RPE65) | Q16518 |
| | Centrosomal protein of 290 kDa | O15078 |
| | (CEP290) | |
| | Protein crumbs homolog 1 (CRB1) | P82279 |
| Leber's hereditary optical neuropathy | NADH-ubiquinone oxidoreductase chain | P03905 |
| | 4 (ND4) | |
| Leukodystrophy | ARSA | P15289 |
| Levine-Critchley Syndrome - | VPS13A | Q96RL7 |
| choreoacanthocytosis | | |
| Lewy body dementia | SNCA | P37840 |
| | SNCB | Q16143 |
| Lipoid Proteinosis - Urbach-Wiethe | ECM1 | Q16610 |
| disease | | |
| Lissencephaly | PAFAH1B1 | Q9PTR5 |
| | NDE1 | Q9NXR1 |
| | TUBA1A | Q71U36 |
| | LAMB1 | LAMB1 |
| | KATNB1 | Q9BVA0 |
| | RELN | P78509 |
| Macrocephaly/Megalencephaly | TBC1D7 | Q9P0N9 |
| Menkes Disease | ATP7A | Q04656 |
| Metachromatic Leukodystrophy - MLD | Arylsulfatase A (ARSA) | P15289 |
| Microcephaly diseases | KIF11 | P52732 |
| | MCPH1 | Q8NEM0 |
| | SLC25A19 | Q9HC21 |
| Migraine, familial hemiplegic | CACNA1A | O00555 |
| | ATP1A2 | P50993 |
| | SCN1A | P35498 |
| Mitochondrial DNA depletion | RRM2B | Q7LG56 |
| syndromes | DGUOK | Q16854 |
| | POLG | P54098 |
| | TYMP | P19971 |
| | TK2 | O00142 |
| Morvan disease | WNK1 | Q9H4A3 |
| Mucolipidosis | GNPTAB | Q3T906 |
| | MCOLN1 | Q9GZU1 |
| Mucopolysaccharidosis Type I (MPS I) - | alpha-L-iduronidase (IDUA) | P35475 |
| Hurler syndrome | | |
| MPS II - Hunter syndrome | iduronate-2-sulfatase (IDS) | P22304 |
| MPS IIIa - Sanfilippo Type A syndrome | heparan sulfate sulfatase (HSS) or N- | P51688 |
| | sulfoglucosamine sulfohydrolase | |
| | (SGSH) | |
| MPS IIIB - Sanfilippo Type B | N-acetyl-alpha-D-glucosaminidase | P54802 |
| syndrome | (NAGLU) | |
| MPS VI - Maroteaux-Lamy syndrome | arylsulfatase B (ARSB) | P15848 |
| MPS IV A - Morquio syndrome type A | N-acetylgalactosamine-6-sulfatase | P34059 |
| | (GALNS) | |
| MPS IV B - Morquio syndrome type B | Beta-galactosidase 1 (GLB1) | P16278 |
| MPS VII - Sly syndrome | beta-glucuronidase | P08236 |
| MPS VIII | glucosamine-6-sulfate sulfatase | P15586 |
| MPS IX | Hyaluronidase-1 (HYAL1) | Q12794 |
| Multiple Sclerosis | PDCD1 | Q15116 |
| Multiple system atrophy | COQ2 | Q96H96 |
| Myasthenic syndrome, congenital | CHAT | P28329 |
| presynaptic | | |
| Myoclonus | NOL3 | O60936 |
| Myoclonic epilepsy (FAME2) | STARD7 | Q9NQZ5 |
| Narcolepsy | HCRT, OX | O43612 |
| | MOG | Q16653 |
| Neuroacanthocytosis - McLeod | XK | P51811 |
| syndrome | | |
| Neurodevelopmental disorder with | TTC5 | Q8N0Z6 |
| cerebral atrophy and facial | | |
| dysmorphism (NEDCAFD) | | |
| Neurodevelopmental disorder with | NCDN | Q9UBB6 |
| infantile epileptic spasms | | |
| Neurofibromatosis | NF1 | P21359 |
| Neuromyotonia | HINT1 | P49773 |
| Neuronal Ceroid Lipofuscinosis | PPT1 | P50897 |
| | TPP1 | O14773 |
| | CLN5 | O75503 |
| | CLN3 | Q13286 |
| | CLN6 | Q9NWW5 |
| | CLN8 | Q9UBY8 |
| | DNAJC5 | Q9H3Z4 |
| | MFSD8 | Q8NHS3 |
| | CTSD | P07339 |
| Neuropathy, ataxia and retinitis | MTATP6 | P00846 |
| pigmentosa (NARP) | | |
| Neuropathy, hereditary sensory and | WNK1 | Q9H4A3 |
| autonomic, type II | | |
| Neuropathy, hypomyelinating | EGR2 | P11161 |
| congenital 1 | | |
| Niemann-Pick disease | Sphingomyelin phosphodiesterase 1 | P17405 |
| | (SMPD1) | |
| | NPC intracellular cholesterol transporter | O15118 |
| | 1 (NPC1) | |
| Ohtahara Syndrome - Developmental | ARX | Q96QS3 |
| and epileptic encephalopathy 1 | | |
| Omithine Transcarbamylase deficiency | OTC | P00480 |
| Orthostatic intolerance | SLC6A2 | P23975 |
| Parkinson's disease | Glucocerebrosidase (GBA1) | P04062 |
| | Dopamine decarboxylase (DDC) | P20711 |
| | Neurturin | Q99748 |
| | Glial derived growth factor (GDGF) | P39905 |
| | Tyrosine hydroxylase (TH), tyrosine 3- | P07101 |
| | monooxygenase | |
| | Glutamic acid decarboxylase (GAD) | Q99259 |
| | Fibroblast growth factor 2 (FGF2) | P09038 |
| | Brain-derived neurotrophic factor | P23560 |
| | (BDNF) | |
| Paroxysmal Choreoathetosis | PNKD | Q8N490 |
| Pelizaeus-Merzbacher Disease | PLP1 | P60201 |
| Pena-Shokeir Type II Syndrome | ERCC6 | Q03468 |
| Periodic Paralyses | SCN4A | P35499 |
| Phelan-McDermid syndrome | SH3 and multiple ankyrin repeat | Q9BYB0 |
| | domains protein 3 (SHANK3) | |
| Phytanic Acid Storage Disease - | PEX1 | O43933 |
| peroxisome biogenesis disorder 1B | | |
| Pick disease | PSEN1 | A0A024R6A3 |
| | MAPT (tau) | P10636 |
| Porencephaly type 1 | COL4A1 | P02462 |
| Primary Lateral Sclerosis, juvenile | ALS2 | Q96Q42 |
| Primary Progressive Aphasia | GRN | P28799 |
| Progressive external ophtalmoplegia | POLG | P54098 |
| | POLG2 | Q9UHN1 |
| | SLC25A4 | P12235 |
| | TWNK | Q96RR1 |
| Progressive bulbar palsy | SLC52A3 | Q9NQ40 |
| Progressive supranuclear palsy | Microtubule-associated protein tau | P10636 |
| | (MAPT), Tau | |
| Pseudo-Torch syndrome | OCLN | Q16625 |
| | STAT2 | P52630 |
| | USP18 | Q9UMW8 |
| Retinitis Pigmentosa 38 - rod-cone | Tyrosine-protein kinase Mer (MERTK) | Q12866 |
| dystrophy | | |
| Retinitis Pigmentosa 40 | PDE6B | P35913 |
| Rett syndrome | Methyl-CpG-binding protein 2 (MECP2) | P51608 |
| Sandhoff disease | Beta-hexosaminidase subunit alpha | P06865 |
| | (HEXA) | |
| | Beta-hexosaminidase subunit beta | P07686 |
| | (HEXB) | |
| Schizencephaly | SIX3 | O95343 |
| | EMX2 | Q04743 |
| | SHH | Q15465 |
| Seitelberger Disease | PLA2G6 | O60733 |
| Septo-optic dysplasia - De Morsier | HESX1 | Q9UBX0 |
| syndrome | | |
| Snijders Blok-Fisher syndrome | POU3F3 | P20264 |
| Spastic Paraplegias | SPG11 | Q96JI7 |
| | SPAST | Q9UBP0 |
| | KIF5A | Q12840 |
| | NIPA1 | Q7RTP0 |
| | CYP7B1 | O75881 |
| | ATL1 | Q8WXF7 |
| Spinal Muscular Atrophy - Kugelberg- | Survival motor neuron protein (SMN), | Q16637 |
| Welander Disease | SMN1 | |
| Spinocerebellar ataxia | Ataxin-1 (ATXN1), SCA1 | P54253 |
| | Ataxin-2 (ATXN2), SCA2 | Q99700 |
| | Ataxin-3 (ATXN3), SCA3 | P54252 |
| | ZFHX3 | Q15911 |
| | CACNA1A | O00555 |
| | ATXN7, SCA7 | O15265 |
| | TMEM240 | Q5SV17 |
| Sporadic Inclusion Body Myositis | Follistatin (FST) | P19883 |
| Steele-Richardson-Olszewski | MAPT(Tau) | P10636 |
| syndrome - Parkinson-dementia | | |
| syndrome | | |
| Stiff-Person Syndrome, congenital | GLRA1 | P23415 |
| | GLRB | P48167 |
| Striatonigral degeneration | NUP62 | P37198 |
| | PDE8B | O95263 |
| | MTATP6 | P00846 |
| | VAC14 | Q08AM6 |
| Sturge-Weber Syndrome | GNAQ | P50148 |
| Subcortical Vascular Encephalopathy - | HTRA1 | Q92743 |
| Cerebral Arteriopathy | | |
| Systemic Lupus Erythematosus | DNASE1L3 | Q13609 |
| | TLR7 | Q9NYK1 |
| Tardive Dyskinesia | CYP2D6 | P10635 |
| Tay-Sachs disease | Beta-hexosaminidase subunit alpha | P06865 |
| | (HEXA) | |
| Tourette Syndrome | HDC | P19113 |
| | SLITRK1 | Q96PX8 |
| Tremor, hereditary type 1 | DRD3 | P35462 |
| Troyer Syndrome | SPART | Q8N0X7 |
| Tuberous Sclerosis | TSC1 | Q92574 |
| | TSC2 | P49815 |
| | IFNG | P01579 |
| Von Hippel-Lindau Disease | VHL | P40337 |
| | CCND1 | P24385 |
| Von Recklinghausen Disease | NF1 | P21359 |
| Werdnig-Hoffman Disease | SMN1 | Q16637 |
| West Syndrome, X-linked | ARX | Q96QS3 |
| Wilson disease | ATP7B | P35670 |
| Wolman's disease - acid lipase | LIPA | P38571 |
| disease | | |
| X-linked adrenoleukodystrophy | ATP-binding cassette sub-family D | P33897 |
| | member 1 (ABCD1) | |
| X-linked Retinoschisis | Retinoschisin (RS1) | O15537 |
| X-Linked Retinitis Pigmentosa | X-linked retinitis pigmentosa GTPase | Q92834 |
| | regulator (RPGR) | |
| X-Linked Spinal and Bulbar Muscular | UBA1 | P22314 |
| Atrophy | | |

TABLE 3
Exemplary Muscle Indications and Transgenes

| | | UniProt |
|---|---|---|
| | | Accession # |
| Indication | Transgene | (Human) |
| Acid maltase deficiency - Glycogen | GAA | P10253 |
| storage disease II | | |
| Advanced heart failure | SERCA2a, ATP2A2 | P16615 |
| Amyotrophic lateral sclerosis (ALS) - | Superoxide dismutase-1 (SOD1) | P00441 |
| Lou Gehrig's disease | | |
| Andersen-Tawil Syndrome | KCNJ2 | P63252 |
| Barth syndrome | TAFAZZIN | Q16635 |
| Becker Muscular Dystrophy (BMD) | DMD | P11532 |
| Becker Myotonia Congenita | CLCN1 | P35523 |
| Bethlem Myopathy | COL6A3 | P12111 |
| | COL6A2 | P12110 |
| | COL6A1 | P12109 |
| Bulbospinal Muscular Atrophy | AR | P10275 |
| Carnitine Deficiency, systemic primary | SLC22A5 | O76082 |
| Carnitine Palmityl Transferase | CPT1A | P50416 |
| Deficiency, type 1 | | |
| Carnitine Palmityl Transferase | CPT2 | P23786 |
| Deficiency, type 2 | | |
| Catecholaminergic polymorphic | Calsequestrin-2 (CASQ2) | O14958 |
| ventricular tachycardia 2 (CPVT2) | | |
| Central Core Disease - congenital | RYR1 | P21817 |
| myopathy, type 1A | | |
| Centronuclear Myopathy type 1 | MTMR14 | Q8NCE2 |
| | DNM2 | O14717 |
| Charcot-Marie-Tooth disease | PMP22 | Q01453 |
| | MPZ | P25189 |
| | DNM2 | P50570 |
| | MFN2 | O95140 |
| | KIF1B | O60333 |
| | SBF2 | Q86WG5 |
| | PNKP | Q96T60 |
| | GDAP1 | Q8TB36 |
| | LMNA | P02545 |
| | FGD4 | Q96M96 |
| | MTMR2 | Q13614 |
| Congenital Muscular Dystrophy - | COL6A3 | P12111 |
| Ullrich disease | COL6A2 | P12110 |
| | COL6A1 | P12109 |
| Congenital Myasthenic Syndromes | COLQ | Q9Y215 |
| | AGRN | O00468 |
| | RAPSN | Q13702 |
| | GFPT1 | Q06210 |
| | SCN4A | P35499 |
| | ALG2 | Q9H553 |
| | ALG14 | Q96F25 |
| | DPAGT1 | Q9H3H5 |
| | CHRNE | Q04844 |
| | CHRNA1 | P02708 |
| | DOK7 | Q18PE1 |
| | CHAT | P28329 |
| Congenital Myopathy | ACTA1 | P68133 |
| | STAC3 | Q96MF2 |
| | TPM3 | P06753 |
| Congenital Myotonic Dystrophy | DMPK | Q09013 |
| Cori Disease - Debrancher Enzyme | AGL | P35573 |
| Deficiency - Forbes Disease | | |
| Danon disease | LAMP2 | P13473 |
| Dejerine-Sottas Disease | MPZ | P25189 |
| | EGR2 | P11161 |
| | PMP22 | Q01453 |
| | PRX | Q9BXMO |
| Distal Muscular Dystrophy, Welander | TIA1 | P31483 |
| Distal Muscular Dystrophy, Miyoshi | DYSF | O75923 |
| Distal myopathy with anterior tibial | DYSF | O75923 |
| onset | | |
| Duchenne Muscular Dystrophy | Dystrophin (DMD) | P11532 |
| | GALGT2, B4GALNT2 | Q8NHYO |
| Dysferlinopathies | Dysferlin (DYSF) | O75923 |
| Emery-Dreifuss Muscular Dystrophy | EMD | P50402 |
| | SYNE1 | Q8NF91 |
| | SYNE2 | Q8WXH0 |
| | TMEM43 | Q9BTV4 |
| | LMNA | P02545 |
| Eulenberg Disease - Paramyotonia | SCN4A | P35499 |
| Congenita | | |
| Facioscapulohumeral Muscular | SMCHD1 | A6NHR9 |
| Dystrophy | LRIF1 | Q5T3J3 |
| Friedreich's ataxia | Frataxin (FXN) | Q16595 |
| Fukuyama Congenital Muscular | FKTN | O75072 |
| Dystrophy | | |
| Glycogenosis Type 10 - Glycogen | PGAM2 | P15259 |
| storage disease X | | |
| Glycogenosis Type 11 - Glycogen | LDHA | P00338 |
| storage disease XI | | |
| Glycogenosis Type 2 - Glycogen | GAA | P10253 |
| storage disease II - Pompe Disease - | | |
| Acid maltase deficiency | | |
| Glycogenosis Type 3 - Glycogen | AGL | P35573 |
| storage disease III | | |
| Glycogenosis Type 5 - Glycogen | PYGM | P11217 |
| storage disease V - McArdle Disease - | | |
| Myophosphorylase Deficiency | | |
| Glycogenosis Type 7 - Glycogen | PFKM | P08237 |
| storage disease VII - | | |
| Phosphofructokinase Deficiency - Tarui | | |
| Disease | | |
| Glycogenosis Type 9 - Glycogen | PHKB | Q93100 |
| storage disease IX | PHKA2 | P46019 |
| Hereditary Inclusion-Body Myositis | GNE | Q9Y223 |
| Integrin-Deficient Congenital Muscular | ITGA7 | Q13683 |
| Dystrophy | | |
| Kennedy Disease - Spinal-Bulbar | Androgen receptor (AR) | P10275 |
| Muscular Atrophy | | |
| Kugelberg-Welander Disease | SMN1 | Q16637 |
| Lactate dehydrogenase A deficiency | LDHA | P00338 |
| Lactate Dehydrogenase B Deficiency | LDHB | P07195 |
| Lambert-Eaton Myasthenic Syndrome | CACNB2 | Q08289 |
| Laing Distal Myopathy | MYH7 | A7E2Y1 |
| Limb Girdle Muscular Dystrophy Type | Gamma-sarcoglycan | Q13326 |
| 2C (LGMD-2C) | | |
| Limb Girdle Muscular Dystrophy Type | Alpha-sarcoglycan | Q16586 |
| 2D (LGMD-2D) | | |
| Limb Girdle Muscular Dystrophy | Beta-sarcoglycan | Q16585 |
| Type2E ( LGMD-2E) | | |
| Limb Girdle Muscular Dystrophy Type | Delta-sarcoglycan | Q92629 |
| 2F (LGMD-2F) | | |
| Merosin-Deficient Congenital Muscular | LAMA2 | P24043 |
| Dystrophy | | |
| Muscle-Eye-Brain Disease | POMGNT1 | Q8WZA1 |
| Mitochondrial Myopathy | CHCHD10 | Q8WYQ3 |
| Miyoshi myopathy | DYSF | O75923 |
| Myoadenylate Deaminase Deficiency | AMPD1 | P23109 |
| Myofibrillar Myopathy 1 | DES | P17661 |
| Myofibrillar Myopathy 2 | CRYAB | P02511 |
| Myofibrillar Myopathy 3 | MYOT | Q9UBF9 |
| Myofibrillar Myopathy 4 - ZASP related | LDB3, ZASP | O75112 |
| myopathy | | |
| Myofibrillar Myopathy 5 | FLNC | Q14315 |
| Myofibrillar Myopathy 6 | BAG3 | 095817 |
| Myofibrillar Myopathy 7 | KY | Q8NBH2 |
| Myofibrillar Myopathy 8 | PYROXD1 | Q8WU10 |
| Myofibrillar Myopathy 9 | TTN | Q8WZ42 |
| Myofibrillar Myopathy 10 | SVIL | O95425 |
| Myofibrillar Myopathy 11 | UNC45B | Q8IWX7 |
| Myofibrillar Myopathy 12 | MYL2 | Q99972 |
| Myotonic dystrophy Type 1 - Steinert | Myotonin-protein kinase (DMPK) | Q09013 |
| Disease | | |
| Myotonic dystrophy Type 2 | CNBP | P62633 |
| Myotubular Myopathy | MTM1 | Q13496 |
| Nemaline Myopathy 1 | TPM3 | P06753 |
| Nemaline Myopathy 2 | NEB | P20929 |
| Nemaline Myopathy 5A, 5B, 5C | TNNT1 | P13805 |
| Nemaline Myopathy 3 | ACTA1 | P68133 |
| Nemaline Myopathy 6 | KBTBD13 | C9JR72 |
| Nemaline Myopathy 4 | TPM2 | P07951 |
| Nemaline Myopathy 7 | CFL2 | Q9Y281 |
| Nemaline Myopathy 8 | KLHL40 | Q2TBAO |
| Nemaline Myopathy 9 | KLHL41 | O60662 |
| Nemaline Myopathy 10 | LMOD3 | QOVAK6 |
| Nonaka Distal Myopathy | GNE | Q9Y223 |
| Oculopharynggeal muscular dystrophy | PABPN1 | Q86U42 |
| Omithine Transcarbamylase deficiency | OTC | P00480 |
| Paramyotonia Congenita | SCN4A | P35499 |
| Periodic Paralysis, hypokalemic | CACNA1S | Q13698 |
| Periodic Paralysis, hyperkalemic | SCN4A | P35499 |
| Phosphoglycerate Kinase Deficiency | PGK1 | P00558 |
| Polymyositis | PMSCL2 | Q01780 |
| | PMSCL1 | Q06265 |
| Progressive External Ophthalmoplegia | POLG | P54098 |
| | POLG2 | Q9UHN1 |
| | SLC25A4 | P12235 |
| | TWNK | Q96RR1 |
| Spinal Muscular Atrophy type 3 - | Survival motor neuron protein (SMN), | Q16637 |
| Kugelberg-Welander Disease | SMN1 | |
| Thomsen Disease - Myotonia | CLCN1 | P35523 |
| Congenita (autosomal dominant) | | |
| Walker-Warburg Syndrome | POMT1 | Q9Y6A1 |
| X-linked myotubular myopathy | Myotubularin (MTM1) | Q13496 |
| Werdnig-Hoffmann Disease - Spinal | SMN1 | Q16637 |
| Muscular Atrophy type 1 | | |

TABLE 4
Additional Exemplary Indications and Transgenes

| | | UniProt |
|---|---|---|
| | | Accession # |
| Indication | Transgene | (Human) |
| Osteogenesis imperfecta | COL1A1 | P02452 |
| (brittle bone disease) Type I, II, | COL1A2 | P08123 |
| III, or IV | | |
| Hereditary angioedema | Plasma protease C1 inhibitor (SERPING1, C1NH) | P05155 |
| Osteogenesis imperfecta Type | Interferon-induced transmembrane protein 5 | A6NNB3 |
| V | (IFITM5, IFM5) | |
| Osteogenesis Imperfecta Type VI | Pigment epithelium-derived factor (SERPINF1, | P36955 |
| | PEDF) | |
| Osteogenesis Imperfecta Type | Cartilage-associated protein (CRTAP) | 075718 |
| VII | | |
| Osteogenesis Imperfecta Type | Prolyl 3-hydroxylase 1 (P3H1, LEPRE1) | Q32P28 |
| VIII | | |
| Osteogenesis Imperfecta Type | PPIB | P23284 |
| IX | | |
| Maple syrup urine disease | BCKDHA | P12694 |
| | BCKDHB | P21953 |
| | DBT | P11182 |
| Alpha-mannosidosis | Lysosomal alpha-mannosidase (MAN2B1) | O00754 |
| Beta-mannosidosis | Beta-mannosidase (MANBA) | O00462 |
| Glycogen storage disease Ia - | Glucose-6-phosphatase catalytic subunit 1 | P35575 |
| Von Gierke disease | (G6PC1) | |
| Bloch-Sulzberger Syndrome | IKBKG | Q9Y6K9 |
| Cholesterol Ester Storage | LIPA | P38571 |
| Disease | | |
| Danon Disease | Lysosome-associated membrane glycoprotein 2 | P13473 |
| | (LAMP2) | |
| Cystic Fibrosis | CTFR | A4L9V0 |
| Rheumatoid Arthritis | TNF | P01375 |
| Alpha-1 Antitrypsin Deficiency | Alpha-1-antitrypsin (Alpha1-AT), AAT, SERPINA1 | P01009 |
| Hemophilia A | Coagulation factor VIII | P00451 |
| Hemophilia B | Coagulation factor IX | P00740 |
| Homozygous Familial | Low-density lipoprotein receptor (LDLR) | P01130 |
| Hypercholesterolemia -FHCL1 | | |
| Mucopolysaccharidosis Type | Arylsulfatase B (ARSB) | P15848 |
| VI | | |
| Familial Lipoprotein Lipase | LPL | P06858 |
| Deficiency | | |
| Fragile X syndrome | FMR1 | Q06787 |

### 5.8. Methods of Treatment

The disclosure is directed, in part, to a method of treating a disease or condition in a subject, e.g., an animal or human subject. In some embodiments, a method of treating a disease or condition in a subject comprises administering to the subject a dependoparvovirus particle comprising a variant polypeptide described herein, e.g., comprising a payload described herein. In some embodiments, the dependoparvovirus particle, which comprises a variant polypeptide, comprising a payload described herein is administered in an amount and/or time effective to treat the disease or condition. In some embodiments, the payload is a therapeutic product. In some embodiments, the payload is a nucleic acid, e.g., encoding an exogenous polypeptide. The disclosure is also directed to a dependoparvovirus particle comprising a variant polypeptide described herein, e.g., comprising a payload described herein, for use in the methods of treatment described herein. The disclosure is also directed to the use of a dependoparvovirus particle comprising a variant polypeptide described herein, e.g., comprising a payload described herein for the manufacture of a medicament for the treatment of a disease or condition as described herein.

The dependoparvovirus particles comprising a variant polypeptide described herein or produced by the methods described herein can be used to express one or more therapeutic proteins to treat various diseases or disorders. In some embodiments, the disease or disorder is a cancer, e.g., a cancer such as carcinoma, sarcoma, leukemia, lymphoma; or an autoimmune disease, e.g., multiple sclerosis. Non-limiting examples of carcinomas include esophageal carcinoma; bronchogenic carcinoma; colon carcinoma; colorectal carcinoma; gastric carcinoma; hepatocellular carcinoma; basal cell carcinoma, squamous cell carcinoma (various tissues); bladder carcinoma, including transitional cell carcinoma; lung carcinoma, including small cell carcinoma and non-small cell carcinoma of the lung; adrenocortical carcinoma; sweat gland carcinoma; sebaceous gland carcinoma; thyroid carcinoma; pancreatic carcinoma; breast carcinoma; ovarian carcinoma; prostate carcinoma; adenocarcinoma; papillary carcinoma; papillary adenocarcinoma; cystadenocarcinoma; medullary carcinoma; renal cell carcinoma; uterine carcinoma; testicular carcinoma; osteogenic carcinoma; ductal carcinoma in situ or bile duct carcinoma; choriocarcinoma; seminoma; embryonal carcinoma; Wilm's tumor; cervical carcinoma; epithelial carcinoma; and nasopharyngeal carcinoma. Non-limiting examples of sarcomas include fibrosarcoma, myxosarcoma, liposarcoma, angiosarcoma, endotheliosarcoma, lymphangiosarcoma, chondrosarcoma, chordoma, osteogenic sarcoma, osteosarcoma, lymphangioendotheliosarcoma, synovioma, mesothelioma, Ewing's sarcoma, leiomyosarcoma, rhabdomyosarcoma, and other soft tissue sarcomas. Non-limiting examples of solid tumors include ependymoma, pinealoma, hemangioblastoma, acoustic neuroma, oligodendroglioma, glioma, astrocytoma, medulloblastoma, craniopharyngioma, menangioma, melanoma, neuroblastoma, and retinoblastoma. Non-limiting examples of leukemias include chronic myeloproliferative syndromes; T-cell CLL prolymphocytic leukemia, acute myelogenous leukemias; chronic lymphocytic leukemias, including B-cell CLL, hairy cell leukemia; and acute lymphoblastic leukemias. Examples of lymphomas include, but are not limited to, B-cell lymphomas, such as Burkitt's lymphoma; and Hodgkin's lymphoma. In some embodiments, the disease or disorder is a genetic disorder. In some embodiments, the genetic disorder is sickle cell anemia, Glycogen storage diseases (GSD, e.g., GSD types I, II, III, IV, V, VI, VII, VIII, IX, X, XI, XII, XIII, and XIV), cystic fibrosis, lysosomal acid lipase (LAL) deficiency 1, Tay-Sachs disease, Phenylketonuria, Mucopolysaccharidoses, Galactosemia, muscular dystrophy (e.g., Duchenne muscular dystrophy), hemophilia such as hemophilia A (classic hemophilia) or hemophilia B (Christmas Disease), Wilson's disease, Fabry Disease, Gaucher Disease hereditary angioedema (HAE), and alpha 1 antitrypsin deficiency. Examples of other diseases or disorders are provided above in Section 5.7.

In some aspects, the disease or condition is a disease of the CNS. Exemplary diseases of the CNS include, Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS—Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Angleman syndrome, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Bechet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbar palsy, Bulbospinal Muscular Atrophy, Cerebral Autosomal Dominant Arteriopathy with Sub-cortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Concentric sclerosis (Bal6's sclerosis), Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Chronic progressive external ophtalmoplegia, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia-Multi-Infarct, Dementia—Semantic, Dementia-Subcortical, Dementia With Lewy Bodies, Demyelination diseases, Dentate Cerebellar Ataxia, Dentatorubral Atrophy, Dermatomyositis, Developmental Dyspraxia, Devic's Syndrome, Diabetic Neuropathy, Diffuse Sclerosis, Distal hereditary motor neuronopathies, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalomyelitis, Encephalopathy, Encephalopathy (familial infantile), Encephalotrigeminal Angiomatosis, Epilepsy, Epileptic Hemiplegia, Episodic ataxia, Erb's Palsy, Erb-Duchenne and Dejerine-Klumpke Palsies, Essential Tremor, Extrapontine Myelinolysis, Faber's disease, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Fragile X syndrome, Friedreich's Ataxia, Frontotemporal Dementia, Gaucher Disease, Generalized Gangliosidoses (GM1, GM2), Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barre Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hurler syndrome, Hydranencephaly, Hydrocephalus, Hydrocephalus —Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kluver-Bucy Syndrome, Korsakoff's Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lichtheim's disease, Lipid Storage Diseases, Lipoid Proteinosis, Lissencephaly, Locked-In Syndrome, Lou Gehrig's Disease, Lupus —Neurological Sequelae, Lyme Disease —Neurological Complications, Lysosomal storage disorders, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Mitochondrial DNA depletion syndromes, Moebius Syndrome, Monomelic Amyotrophy, Morvan Syndrome, Motor Neuron Diseases, Moyamoya Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia Congenital, Myasthenia Gravis, Myelinoclastic Diffuse Sclerosis, Myelitis, Myoclonic Encephalopathy of Infants, Myoclonus, Myoclonus epilepsy, Myopathy, Myopathy—Congenital, Myopathy-Thyrotoxic, Myotonia, Myotonia Congenita, Narcolepsy, NARP (neuropathy, ataxia and retinitis pigmentosa), Neuroacanthocytosis, Neurodegeneration with Brain Iron Accumulation, Neurodegenerative disease, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathic pain, Neuropathy—Hereditary, Neuropathy, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain-Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Peroneal muscular atrophy, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive bulbar palsy, Progressive Hemifacial Atrophy, Progressive Locomotor Ataxia, Progressive Multifocal Leukoencephalopathy, Progressive Muscular Atrophy, Progressive Sclerosing Poliodystrophy, Progressive Supranuclear Palsy, Prosopagnosia, Pseudobulbar palsy, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease —Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjogren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Ataxia, Spinocerebellar Atrophy, Spinocerebellar Degeneration, Sporadic ataxia, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Vitamin B12 deficiency, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, X-Linked Spinal and Bulbar Muscular Atrophy. Examples of other diseases or disorders are provided above in Section 5.7.

In some aspects, the disease or condition is a disease of skeletal muscle. Exemplary diseases of the skeletal muscle include Acid Maltase Deficiency (AMD), Amyotrophic Lateral Sclerosis (ALS), Andersen-Tawil Syndrome, Becker Muscular Dystrophy (BMD), Becker Myotonia Congenita, Bethlem Myopathy, Bulbospinal Muscular Atrophy (Spinal—Bulbar Muscular Atrophy), Carnitine Deficiency, Carnitine Palmityl Transferase Deficiency (CPT Deficiency), Central Core Disease (CCD), Centronuclear Myopathy, Charcot-Marie-Tooth Disease (CMT), Congenital Muscular Dystrophy (CMD), Congenital Myasthenic Syndromes (CMS), Congenital Myotonic Dystrophy, Cori Disease (Debrancher Enzyme Deficiency), Debrancher Enzyme Deficiency, Dejerine-Sottas Disease (DSD), Dermatomyositis (DM), Distal Muscular Dystrophy (DD), Duchenne Muscular Dystrophy (DMD), Dystrophia Myotonica (Myotonic Muscular Dystrophy), Emery-Dreifuss Muscular Dystrophy (EDMD), Endocrine Myopathies, Eulenberg Disease (Paramyotonia Congenita), Facioscapulohumeral Muscular Dystrophy (FSH or FSHD), Finnish (Tibial) Distal Myopathy, Forbes Disease (Debrancher Enzyme Deficiency), Friedreich's Ataxia (FA), Fukuyama Congenital Muscular Dystrophy, Glycogenosis Type 10, Glycogenosis Type 11, Glycogenosis Type 2, Glycogenosis Type 3, Glycogenosis Type 5, Glycogenosis Type 7, Glycogenosis Type 9, Gowers-Laing Distal Myopathy, Hauptmann-Thanheuser MD (Emery—Dreifuss Muscular Dystrophy), Hereditary Inclusion-Body Myositis, Hereditary Motor and Sensory Neuropathy (Charcot-Marie-Tooth Disease), Hyperthyroid Myopathy, Hypothyroid Myopathy, Inclusion-Body Myositis (IBM), Inherited Myopathies, Integrin-Deficient Congenital Muscular Dystrophy, Kennedy Disease (Spinal-Bulbar Muscular Atrophy), Kugelberg-Welander Disease (Spinal Muscular Atrophy), Lactate Dehydrogenase Deficiency, Lambert-Eaton Myasthenic Syndrome (LEMS), Limb-Girdle Muscular Dystrophy (LGMD), Lou Gehrig's Disease (Amyotrophic Lateral Sclerosis), McArdle Disease (Phosphorylase Deficiency), Merosin-Deficient Congenital Muscular Dystrophy, Metabolic Diseases of Muscle, Mitochondrial Myopathy, Miyoshi Distal Myopathy, Motor Neurone Disease, Muscle-Eye-Brain Disease, Myasthenia Gravis (MG), Myoadenylate Deaminase Deficiency, Myofibrillar Myopathy, Myophosphorylase Deficiency, Myotonia Congenita (MC), Myotonic Muscular Dystrophy (MMD), Myotubular Myopathy (MTM or MM), Nemaline Myopathy, Nonaka Distal Myopathy, Oculopharyngeal Muscular Dystrophy (OPMD), Paramyotonia Congenita, Pearson Syndrome, Periodic Paralysis, Peroneal Muscular Atrophy (Charcot-Marie-Tooth Disease), Phosphofructokinase Deficiency, Phosphoglycerate Kinase Deficiency, Phosphoglycerate Mutase Deficiency, Phosphorylase Deficiency, Phosphorylase Deficiency, Polymyositis (PM), Pompe Disease (Acid Maltase Deficiency), Progressive External Ophthalmoplegia (PEO), Rod Body Disease (Nemaline Myopathy), Spinal Muscular Atrophy (SMA), Spinal-Bulbar Muscular Atrophy (SBMA), Steinert Disease (Myotonic Muscular Dystrophy), Tarui Disease (Phosphofructokinase Deficiency), Thomsen Disease (Myotonia Congenita), Ullrich Congenital Muscular Dystrophy, Walker-Warburg Syndrome (Congenital Muscular Dystrophy), Welander Distal Myopathy, Werdnig-Hoffmann Disease (Spinal Muscular Atrophy), and ZASP-Related Myopathy.

In some aspects, the disease or condition is a disease affecting the CNS and muscle, for example SMA, multiple sclerosis, Amyotrophic lateral sclerosis (ALS), Ataxia, Becker muscular dystrophy, Charcot-Marie-Tooth disease, Dystonias, Friedreich's ataxia, Glycogen storage disease II, Kennedy Disease, Lambert-Eaton Myasthenic Syndrome, Mitochondrial DNA depletion syndromes, Muscle-Eye-Brain Disease, Neuromyotonia, Periodic Paralyses, juvenile Primary Lateral Sclerosis, Progressive external ophtalmoplegia, Spastic Paraplegias, congenital Stiff-Person Syndrome, Tardive Dyskinesia, Werdnig-Hoffman Disease, or X-Linked Spinal and Bulbar Muscular Atrophy.

In some embodiments, administration of a dependoparvovirus particle comprising a variant polypeptide and comprising a payload (e.g., a transgene as described in Section 5.7 or its subparts) to a subject induces expression of the payload (e.g., transgene) in a subject. In some embodiments, the expression is induced in the CNS. In some embodiments, the production is similar in the CNS as compared to a similar particle with the wild-type capsid protein. In some embodiments, the production is increased in the CNS as compared to a similar particle with the wild-type capsid protein, for example, a particle with capsid proteins of SEQ ID NO:1. In some embodiments, the expression is induced in skeletal muscle. In some embodiments, the production is similar in muscle as compared to a similar particle with the wild-type capsid protein, for example, a particle with capsid proteins of SEQ ID NO:1. In some embodiments, the production is increased in skeletal muscle as compared to a similar particle with the wild-type capsid protein (for example, a particle with capsid proteins of SEQ ID NO:1). The amount of a payload, e.g., transgene, e.g., heterologous protein, e.g., therapeutic polypeptide, expressed in a subject (e.g., the serum of the subject) can vary. For example, in some embodiments the payload, e.g., protein or RNA product of a transgene, can be expressed in the serum of the subject in the amount of at least about 9 μg/ml, at least about 10 μg/ml, at least about 50 μg/ml, at least about 100 μg/ml, at least about 200 μg/ml, at least about 300 μg/ml, at least about 400 μg/ml, at least about 500 μg/ml, at least about 600 μg/ml, at least about 700 μg/ml, at least about 800 μg/ml, at least about 900 μg/ml, or at least about 1000 μg/ml. In some embodiments, the payload, e.g., protein or RNA product of a transgene, is expressed in the serum of the subject in the amount of about 9 μg/ml, about 10 μg/ml, about 50 μg/ml, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1000 μg/ml, about 1500 μg/ml, about 2000 μg/ml, about 2500 μg/ml, or a range between any two of these values.

In some embodiments, for therapeutic applications, a viral particle comprising a capsid polypeptide as described herein is prepared as a pharmaceutical composition. As used herein the term “pharmaceutical composition” refers to a composition comprising at least one active ingredient (e.g., the viral particle) and optionally, one or more pharmaceutically acceptable carriers or excipients.

Relative amounts of the active ingredient, pharmaceutically acceptable carrier or excipient, and/or any additional ingredients in a pharmaceutical composition in accordance with the present disclosure may vary. Differences in the constitution of a pharmaceutical composition may depend upon the identity, size, and/or condition of the subject being treated, the route by which the composition is to be administered, and/or any other factor. The composition may comprise between 0.0001% and 99% (w/w) of the active ingredient. By way of example, the composition may comprise between 0.0001% and 100%, e.g., between 0.5 and 50%, between 1-30%, between 5-80%, or at least 80% (w/w) active ingredient. Non limiting examples of carriers and/or excipients include solvents, dispersion media, diluents, or other liquid vehicles, dispersion or suspension aids, surface active agents, isotonic agents, thickening or emulsifying agents, preservatives, or combination thereof.

### 6. NUMBERED EMBODIMENTS

While various specific embodiments have been illustrated and described, it will be appreciated that various changes can be made without departing from the spirit and scope of the disclosure(s). The present disclosure is exemplified by the numbered embodiments set forth below. Unless otherwise specified, features of any of the concepts, aspects and/or embodiments described in the detailed description above are applicable mutatis mutandis to any of the following numbered embodiments.

Various numbered embodiments below refer to virus particles, and it will be understood that such viral particles are engineered particles comprising a capsid polypeptide and a nucleic acid that do not naturally occur together in nature. Further, the nucleic acid can include components that do not naturally occur together in nature. For example, a nucleic acid can comprise a payload (e.g., a nucleotide sequence of a transgene, which may encode a non-naturally occurring, e.g., variant, polypeptide), one or more regulatory elements (which may comprise a non-naturally occurring, e.g., variant, regulatory sequence such as a promoter), and AAV ITRs, where two or more of such components do not naturally occur together in nature. In some embodiments, (a) a viral particle comprises a variant capsid polypeptide of a first serotype (e.g., a variant AAV9 capsid polypeptide) and ITRs from the genome of an AAV of a different serotype (e.g., AAV2 ITRs) and/or (b) the payload is a transgene encoding a human polypeptide or a variant thereof.

1. A capsid polypeptide comprising:
(a) a leucine at a position corresponding to V596 of the VP1 capsid polypeptide of SEQ ID NO:1; and
(b) a serine or threonine at a position corresponding to N598 of the VP1 capsid polypeptide of SEQ ID NO:1.
   2. In a capsid polypeptide, the improvement comprising:
(a) a leucine at a position corresponding to V596 of the VP1 capsid polypeptide of SEQ ID NO:1; and
(b) a serine or threonine at a position corresponding to N598 of the VP1 capsid polypeptide of SEQ ID NO:1.
   3. The capsid polypeptide of embodiment 1 or embodiment 2, which comprises a serine at a position corresponding to N598 of the VP1 capsid polypeptide of SEQ ID NO:1.
   4. The capsid polypeptide of embodiment 1 or embodiment 2, which comprises a threonine at a position corresponding to N598 of the VP1 capsid polypeptide of SEQ ID NO:1.
   5. The capsid polypeptide of any one of embodiments 1 to 4, which does not comprise a valine at a position corresponding to T593 of the VP1 capsid polypeptide of SEQ ID NO:1.
   6. The capsid polypeptide of any one of embodiments 1 to 5, which does not comprise an alanine at a position corresponding to W595 of the VP1 capsid polypeptide of SEQ ID NO:1.
   7. A capsid polypeptide comprising:
(a) an alanine, arginine, serine, threonine, or valine at a position corresponding to T593 of the VP1 capsid polypeptide of SEQ ID NO:1;
(b) a leucine at a position corresponding to V596 of the VP1 capsid polypeptide of SEQ ID NO:1; and
(c) a serine or threonine at a position corresponding to N598 of the VP1 capsid polypeptide of SEQ ID NO:1.
   8. In a capsid polypeptide, the improvement comprising:
(a) an alanine, arginine, serine, threonine, or valine at a position corresponding to T593 of the VP1 capsid polypeptide of SEQ ID NO:1;
(b) a leucine at a position corresponding to V596 of the VP1 capsid polypeptide of SEQ ID NO:1; and
(c) a serine or threonine at a position corresponding to N598 of the VP1 capsid polypeptide of SEQ ID NO:1
   9. The capsid polypeptide of embodiment 7 or embodiment 8, which comprises an alanine at a position corresponding to T593 of the VP1 capsid polypeptide of SEQ ID NO:1.
   10. The capsid polypeptide of embodiment 7 or embodiment 8, which comprises an arginine at a position corresponding to T593 of the VP1 capsid polypeptide of SEQ ID NO:1.
   11. The capsid polypeptide of embodiment 7 or embodiment 8, which comprises a serine at a position corresponding to T593 of the VP1 capsid polypeptide of SEQ ID NO:1.
   12. The capsid polypeptide of embodiment 7 or embodiment 8, which comprises a threonine at a position corresponding to T593 of the VP1 capsid polypeptide of SEQ ID NO:1.
   13. The capsid polypeptide of embodiment 7 or embodiment 8, which comprises a valine at a position corresponding to T593 of the VP1 capsid polypeptide of SEQ ID NO:1.
   14. The capsid polypeptide of any one of embodiments 7 to 13, which comprises a serine at a position corresponding to N598 of the VP1 capsid polypeptide of SEQ ID NO:1.
   15. The capsid polypeptide of any one of embodiments 7 to 13, which comprises a threonine at a position corresponding to N598 of the VP1 capsid polypeptide of SEQ ID NO:1.
   16. The capsid polypeptide of any one of embodiments 7 to 15, which does not comprise an alanine at a position corresponding to W595 of the VP1 capsid polypeptide of SEQ ID NO:1.
   17. A capsid polypeptide comprising:
(a) an alanine, tryptophan, or tyrosine at a position corresponding to W595 of the VP1 capsid polypeptide of SEQ ID NO: 1
(b) a leucine at a position corresponding to V596 of the VP1 capsid polypeptide of SEQ ID NO:1; and
(c) a serine or threonine at a position corresponding to N598 of the VP1 capsid polypeptide of SEQ ID NO:1.
   18. In a capsid polypeptide, the improvement comprising:
(a) an alanine, tryptophan, or tyrosine at a position corresponding to W595 of the VP1 capsid polypeptide of SEQ ID NO: 1
(b) a leucine at a position corresponding to V596 of the VP1 capsid polypeptide of SEQ ID NO:1; and
(c) a serine or threonine at a position corresponding to N598 of the VP1 capsid polypeptide of SEQ ID NO:1.
   19. The capsid polypeptide of embodiment 17 or embodiment 18, which comprises an alanine at a position corresponding to W595 of the VP1 capsid polypeptide of SEQ ID NO:1.
   20. The capsid polypeptide of embodiment 17 or embodiment 18, which comprises a tryptophan at a position corresponding to W595 of the VP1 capsid polypeptide of SEQ ID NO:1.
   21. The capsid polypeptide of embodiment 17 or embodiment 18, which comprises a tyrosine at a position corresponding to W595 of the VP1 capsid polypeptide of SEQ ID NO:1.
   22. The capsid polypeptide of any one of embodiments 17 to 21, which comprises a serine at a position corresponding to N598 of the VP1 capsid polypeptide of SEQ ID NO:1.
   23. The capsid polypeptide of any one of embodiments 17 to 21, which comprises a threonine at a position corresponding to N598 of the VP1 capsid polypeptide of SEQ ID NO:1.
   24. The capsid polypeptide of any one of embodiments 17 to 23, which does not comprise a valine at a position corresponding to T593 of the VP1 capsid polypeptide of SEQ ID NO:1.
   25. A capsid polypeptide comprising:
(a) an alanine, arginine, serine, threonine, or valine at a position corresponding to T593 of the VP1 capsid polypeptide of SEQ ID NO:1;
(b) an alanine, tryptophan, or tyrosine at a position corresponding to W595 of the VP1 capsid polypeptide of SEQ ID NO: 1
(c) a leucine at a position corresponding to V596 of the VP1 capsid polypeptide of SEQ ID NO:1; and
(d) a serine or threonine at a position corresponding to N598 of the VP1 capsid polypeptide of SEQ ID NO:1.
   26. In a capsid polypeptide, the improvement comprising:
(a) an alanine, arginine, serine, threonine, or valine at a position corresponding to T593 of the VP1 capsid polypeptide of SEQ ID NO:1;
(b) an alanine, tryptophan, or tyrosine at a position corresponding to W595 of the VP1 capsid polypeptide of SEQ ID NO: 1
(c) a leucine at a position corresponding to V596 of the VP1 capsid polypeptide of SEQ ID NO:1; and
(d) a serine or threonine at a position corresponding to N598 of the VP1 capsid polypeptide of SEQ ID NO:1.
   27. The capsid polypeptide of embodiment 25 or embodiment 26, which comprises an alanine at a position corresponding to T593 of the VP1 capsid polypeptide of SEQ ID NO:1.
   28. The capsid polypeptide of embodiment 25 or embodiment 26, which comprises an arginine at a position corresponding to T593 of the VP1 capsid polypeptide of SEQ ID NO:1.
   29. The capsid polypeptide of embodiment 25 or embodiment 26, which comprises a serine at a position corresponding to T593 of the VP1 capsid polypeptide of SEQ ID NO:1.
   30. The capsid polypeptide of embodiment 25 or embodiment 26, which comprises a threonine at a position corresponding to T593 of the VP1 capsid polypeptide of SEQ ID NO:1.
   31. The capsid polypeptide of embodiment 25 or embodiment 26, which comprises a valine at a position corresponding to T593 of the VP1 capsid polypeptide of SEQ ID NO:1.
   32. The capsid polypeptide of any one of embodiments 25 to 31, which comprises an alanine at a position corresponding to W595 of the VP1 capsid polypeptide of SEQ ID NO:1.
   33. The capsid polypeptide of any one of embodiments 25 to 31, which comprises a tryptophan at a position corresponding to W595 of the VP1 capsid polypeptide of SEQ ID NO:1.
   34. The capsid polypeptide of any one of embodiments 25 to 31, which comprises a tyrosine at a position corresponding to W595 of the VP1 capsid polypeptide of SEQ ID NO:1.
   35. The capsid polypeptide of any one of embodiments 25 to 34, which comprises a serine at a position corresponding to N598 of the VP1 capsid polypeptide of SEQ ID NO:1.
   36. The capsid polypeptide of any one of embodiments 25 to 34, which comprises a threonine at a position corresponding to N598 of the VP1 capsid polypeptide of SEQ ID NO:1.
   37. A capsid polypeptide comprising:
(a) a valine at a position corresponding to T593 of the VP1 capsid polypeptide of SEQ ID NO:1;
(b) an alanine at a position corresponding to W595 of the VP1 capsid polypeptide of SEQ ID NO:1;
(c) a leucine at a position corresponding to V596 of the VP1 capsid polypeptide of SEQ ID NO:1; and
(d) a serine at a position corresponding to N598 of the VP1 capsid polypeptide of SEQ ID NO:1.
   38. In a capsid polypeptide, the improvement comprising:
(a) a valine at a position corresponding to T593 of the VP1 capsid polypeptide of SEQ ID NO:1;
(b) an alanine at a position corresponding to W595 of the VP1 capsid polypeptide of SEQ ID NO:1;
(c) a leucine at a position corresponding to V596 of the VP1 capsid polypeptide of SEQ ID NO:1; and
(d) a serine at a position corresponding to N598 of the VP1 capsid polypeptide of SEQ ID NO:1.
   39. The capsid polypeptide of any one of embodiments 1 to 38, which does not comprise an alanine at a position corresponding to 1601 of the VP1 capsid polypeptide of SEQ ID NO:1.
   40. The capsid polypeptide of any one of embodiments 1 to 38, which comprises an alanine at a position corresponding to 1601 of the VP1 capsid polypeptide of SEQ ID NO:1.
   41. The capsid polypeptide of any one of embodiments 1 to 38, which comprises an isoleucine at a position corresponding to 1601 of the VP1 capsid polypeptide of SEQ ID NO:1.
   42. The capsid polypeptide of any one of embodiments 1 to 38, which comprises a valine at a position corresponding to 1601 of the VP1 capsid polypeptide of SEQ ID NO:1.
   43. A capsid polypeptide comprising a valine at a position corresponding to 1601 of the VP1 capsid polypeptide of SEQ ID NO:1, and optionally one, two or three or all four of: (a) an alanine, arginine, serine, threonine, or valine at a position corresponding to T593 of the VP1 capsid polypeptide of SEQ ID NO:1;
(b) an alanine, tryptophan, or tyrosine at a position corresponding to W595 of the VP1 capsid polypeptide of SEQ ID NO: 1
(c) a leucine at a position corresponding to V596 of the VP1 capsid polypeptide of SEQ ID NO:1; and
(d) a serine or threonine at a position corresponding to N598 of the VP1 capsid polypeptide of SEQ ID NO:1.
   44. In a capsid polypeptide, the improvement comprising a valine at a position corresponding to 1601 of the VP1 capsid polypeptide of SEQ ID NO:1, and optionally one, two or three or all four of: (a) an alanine, arginine, serine, threonine, or valine at a position corresponding to T593 of the VP1 capsid polypeptide of SEQ ID NO:1;
(b) an alanine, tryptophan, or tyrosine at a position corresponding to W595 of the VP1 capsid polypeptide of SEQ ID NO: 1
(c) a leucine at a position corresponding to V596 of the VP1 capsid polypeptide of SEQ ID NO:1; and
(d) a serine or threonine at a position corresponding to N598 of the VP1 capsid polypeptide of SEQ ID NO:1.
   45. The capsid polypeptide of any one of embodiments 1 to 44, which comprises an alanine at a position corresponding to Q592 of the VP1 capsid polypeptide of SEQ ID NO:1.
   46. The capsid polypeptide of any one of embodiments 1 to 44, which comprises an asparagine at a position corresponding to Q592 of the VP1 capsid polypeptide of SEQ ID NO:1.
   47. The capsid polypeptide of any one of embodiments 1 to 44, which comprises a glutamine at a position corresponding to Q592 of the VP1 capsid polypeptide of SEQ ID NO:1.
   48. The capsid polypeptide of any one of embodiments 1 to 44, which does not comprise an isoleucine at a position corresponding to Q592 of the VP1 capsid polypeptide of SEQ ID NO:1.
   49. The capsid polypeptide of any one of embodiments 1 to 44, which comprises an isoleucine at a position corresponding to Q592 of the VP1 capsid polypeptide of SEQ ID NO:1.
   50. The capsid polypeptide of any one of embodiments 1 to 44, which comprises a serine at a position corresponding to Q592 of the VP1 capsid polypeptide of SEQ ID NO:1.
   51. The capsid polypeptide of any one of embodiments 1 to 44, which comprises a glycine at a position corresponding to G549 of the VP1 capsid polypeptide of SEQ ID NO:1.
   52. The capsid polypeptide of any one of embodiments 1 to 44, which does not comprise a glycine at a position corresponding to G549 of the VP1 capsid polypeptide of SEQ ID NO:1.
   53. The capsid polypeptide of any one of embodiments 1 to 44, which comprises an alanine, a phenylalanine, a histidine, an isoleucine, an asparagine, a threonine or a tyrosine at a position corresponding to G549 of the VP1 capsid polypeptide of SEQ ID NO:1.
   54. The capsid polypeptide of embodiment 54, which comprises an alanine at a position corresponding to G549 of the VP1 capsid polypeptide of SEQ ID NO:1.
   55. The capsid polypeptide of embodiment 54, which comprises a phenylalanine at a position corresponding to G549 of the VP1 capsid polypeptide of SEQ ID NO:1.
   56. The capsid polypeptide of embodiment 54, which comprises a histidine at a position corresponding to G549 of the VP1 capsid polypeptide of SEQ ID NO:1.
   57. The capsid polypeptide of embodiment 54, which comprises an asparagine at a position corresponding to G549 of the VP1 capsid polypeptide of SEQ ID NO:1.
   58. The capsid polypeptide of embodiment 54, which comprises a threonine at a position corresponding to G549 of the VP1 capsid polypeptide of SEQ ID NO:1.
   59. The capsid polypeptide of embodiment 54, which comprises a tyrosine at a position corresponding to G549 of the VP1 capsid polypeptide of SEQ ID NO:1.
   60. The capsid polypeptide of any one of embodiments 1 to 59, which comprises a tyrosine at a position corresponding to G549 of the VP1 capsid polypeptide of SEQ ID NO:1.
   61. The capsid polypeptide of any one of embodiments 1 to 60, which comprises an arginine at a position corresponding to R550 of the VP1 capsid polypeptide of SEQ ID NO:1.
   62. The capsid polypeptide of any one of embodiments 1 to 60, which comprises a lysine, a leucine or an asparagine at a position corresponding to R550 of the VP1 capsid polypeptide of SEQ ID NO:1.
   63. The capsid polypeptide of embodiment 62, which comprises a lysine at a position corresponding to R550 of the VP1 capsid polypeptide of SEQ ID NO:1
   64. The capsid polypeptide of embodiment 62, which comprises a leucine at a position corresponding to R550 of the VP1 capsid polypeptide of SEQ ID NO:1
   65. The capsid polypeptide of embodiment 62, which comprises an asparagine at a position corresponding to R550 of the VP1 capsid polypeptide of SEQ ID NO:1.
   66. The capsid polypeptide of any one of embodiments 1 to 65, which comprises an aspartic acid at a position corresponding to D551 of the VP1 capsid polypeptide of SEQ ID NO:1.
   67. The capsid polypeptide of any one of embodiments 1 to 65, which comprises a glutamic acid at a position corresponding to D551 of the VP1 capsid polypeptide of SEQ ID NO:1.
   68. The capsid polypeptide of any one of embodiments 1 to 67, which comprises an asparagine at a position corresponding to N552 of the VP1 capsid polypeptide of SEQ ID NO:1.
   69. The capsid polypeptide of any one of embodiments 1 to 68, which comprises a valine at a position corresponding to V553 of the VP1 capsid polypeptide of SEQ ID NO:1.
   70. The capsid polypeptide of any one of embodiments 1 to 68, which comprises a serine at a position corresponding to V553 of the VP1 capsid polypeptide of SEQ ID NO:1.
   71. The capsid polypeptide of any one of embodiments 1 to 70, which comprises an aspartic acid at a position corresponding to D554 of the VP1 capsid polypeptide of SEQ ID NO:1.
   72. The capsid polypeptide of any one of embodiments 1 to 71, which comprises an alanine at a position corresponding to A555 of the VP1 capsid polypeptide of SEQ ID NO:1.
   73. The capsid polypeptide of any one of embodiments 1 to 72, which comprises an aspartic acid at a position corresponding to D556 of the VP1 capsid polypeptide of SEQ ID NO:1.
   74. The capsid polypeptide of any one of embodiments 1 to 73, which comprises a lysine acid at a position corresponding to K557 of the VP1 capsid polypeptide of SEQ ID NO:1.
   75. The capsid polypeptide of any one of embodiments 1 to 73, which comprises a leucine at a position corresponding to K557 of the VP1 capsid polypeptide of SEQ ID NO:1.
   76. The capsid polypeptide of any one of embodiments 1 to 73, which comprises an asparagine at a position corresponding to K557 of the VP1 capsid polypeptide of SEQ ID NO:1.
   77. The capsid polypeptide of any one of embodiments 1 to 76, which comprises a valine at a position corresponding to V558 of the VP1 capsid polypeptide of SEQ ID NO:1.
   78. The capsid polypeptide of any one of embodiments 1 to 77, which comprises a methionine at a position corresponding to M559 of the VP1 capsid polypeptide of SEQ ID NO:1.
   79. The capsid polypeptide of any one of embodiments 1 to 77, which does not comprise a methionine at a position corresponding to M559 of the VP1 capsid polypeptide of SEQ ID NO:1.
   80. The capsid polypeptide of any one of embodiments 1 to 77, which comprises an alanine, a cysteine, an isoleucine, an asparagine, a glutamine, a serine, a threonine or a valine at a position corresponding to M559 of the VP1 capsid polypeptide of SEQ ID NO:1.
   81. The capsid polypeptide of embodiment 80, which comprises an alanine at a position corresponding to M559 of the VP1 capsid polypeptide of SEQ ID NO:1.
   82. The capsid polypeptide of embodiment 80, which comprises a cysteine at a position corresponding to M559 of the VP1 capsid polypeptide of SEQ ID NO:1.
   83. The capsid polypeptide of embodiment 80, which comprises an isoleucine at a position corresponding to M559 of the VP1 capsid polypeptide of SEQ ID NO:1.
   84. The capsid polypeptide of embodiment 80, which comprises an asparagine at a position corresponding to M559 of the VP1 capsid polypeptide of SEQ ID NO:1.
   85. The capsid polypeptide of embodiment 80, which comprises a glutamine at a position corresponding to M559 of the VP1 capsid polypeptide of SEQ ID NO:1.
   86. The capsid polypeptide of embodiment 80, which comprises a serine at a position corresponding to M559 of the VP1 capsid polypeptide of SEQ ID NO:1.
   87. The capsid polypeptide of embodiment 80, which comprises a threonine at a position corresponding to M559 of the VP1 capsid polypeptide of SEQ ID NO:1.
   88. The capsid polypeptide of embodiment 80, which comprises a valine at a position corresponding to M559 of the VP1 capsid polypeptide of SEQ ID NO:1.
   89. The capsid polypeptide of any one of embodiments 1 to 80, which comprises an alanine or a glutamine at a position corresponding to M559 of the VP1 capsid polypeptide of SEQ ID NO:1.
   90. The capsid polypeptide of any one of embodiments 1 to 89, which comprises an isoleucine at a position corresponding to 1560 of the VP1 capsid polypeptide of SEQ ID NO:1.
   91. The capsid polypeptide of any one of embodiments 1 to 89, which comprises a leucine at a position corresponding to 1560 of the VP1 capsid polypeptide of SEQ ID NO:1.
   92. The capsid polypeptide of any one of embodiments 1 to 89, which comprises a glutamine at a position corresponding to 1560 of the VP1 capsid polypeptide of SEQ ID NO:1.
   93. The capsid polypeptide of any one of embodiments 1 to 92, which comprises a threonine at a position corresponding to T561 of the VP1 capsid polypeptide of SEQ ID NO:1.
   94. The capsid polypeptide of any one of embodiments 1 to 92, which comprises a serine at a position corresponding to T561 of the VP1 capsid polypeptide of SEQ ID NO:1.
   95. The capsid polypeptide of any one of embodiments 1 to 94, which comprises an asparagine at a position corresponding to N562 of the VP1 capsid polypeptide of SEQ ID NO:1.
   96. The capsid polypeptide of any one of embodiments 1 to 95, which comprises a glutamic acid at a position corresponding to E563 of the VP1 capsid polypeptide of SEQ ID NO:1.
   97. The capsid polypeptide of any one of embodiments 1 to 96, which comprises a glutamic acid at a position corresponding to E564 of the VP1 capsid polypeptide of SEQ ID NO:1.
   98. The capsid polypeptide of any one of embodiments 1 to 97, which comprises a glutamic acid at a position corresponding to E565 of the VP1 capsid polypeptide of SEQ ID NO:1.
   99. The capsid polypeptide of any one of embodiments 1 to 98, which comprises an isoleucine at a position corresponding to 1566 of the VP1 capsid polypeptide of SEQ ID NO:1.
   100. The capsid polypeptide of any one of embodiments 1 to 99, which comprises a lysine at a position corresponding to K567 of the VP1 capsid polypeptide of SEQ ID NO:1.
   101. The capsid polypeptide of any one of embodiments 1 to 100, which comprises a threonine at a position corresponding to T568 of the VP1 capsid polypeptide of SEQ ID NO:1.
   102. The capsid polypeptide of any one of embodiments 1 to 101, which comprises a threonine at a position corresponding to T569 of the VP1 capsid polypeptide of SEQ ID NO:1.
   103. The capsid polypeptide of any one of embodiments 1 to 102, which comprises an asparagine at a position corresponding to N570 of the VP1 capsid polypeptide of SEQ ID NO:1.
   104. The capsid polypeptide of any one of embodiments 1 to 103, which comprises a proline at a position corresponding to P571 of the VP1 capsid polypeptide of SEQ ID NO:1.
   105. The capsid polypeptide of any one of embodiments 1 to 104, which comprises a valine at a position corresponding to V572 of the VP1 capsid polypeptide of SEQ ID NO:1.
   106. The capsid polypeptide of any one of embodiments 1 to 105, which comprises an alanine at a position corresponding to A573 of the VP1 capsid polypeptide of SEQ ID NO:1.
   107. The capsid polypeptide of any one of embodiments 1 to 106, which comprises a threonine at a position corresponding to T574 of the VP1 capsid polypeptide of SEQ ID NO:1.
   108. The capsid polypeptide of any one of embodiments 1 to 107, which comprises a glutamic acid at a position corresponding to E575 of the VP1 capsid polypeptide of SEQ ID NO:1.
   109. The capsid polypeptide of any one of embodiments 1 to 107, which comprises a serine at a position corresponding to E575 of the VP1 capsid polypeptide of SEQ ID NO:1.
   110. The capsid polypeptide of any one of embodiments 1 to 107, which comprises a tryptophan at a position corresponding to E575 of the VP1 capsid polypeptide of SEQ ID NO:1.
   111. The capsid polypeptide of any one of embodiments 1 to 110, which comprises a serine at a position corresponding to S576 of the VP1 capsid polypeptide of SEQ ID NO:1.
   112. The capsid polypeptide of any one of embodiments 1 to 110, which comprises a tryptophan at a position corresponding to S576 of the VP1 capsid polypeptide of SEQ ID NO:1.
   113. The capsid polypeptide of any one of embodiments 1 to 112, which comprises a tyrosine at a position corresponding to Y577 of the VP1 capsid polypeptide of SEQ ID NO:1.
   114. The capsid polypeptide of any one of embodiments 1 to 112, which comprises a histidine at a position corresponding to Y577 of the VP1 capsid polypeptide of SEQ ID NO:1.
   115. The capsid polypeptide of any one of embodiments 1 to 112, which comprises a threonine at a position corresponding to Y577 of the VP1 capsid polypeptide of SEQ ID NO:1.
   116. The capsid polypeptide of any one of embodiments 1 to 115, which comprises a glycine at a position corresponding to G578 of the VP1 capsid polypeptide of SEQ ID NO:1.
   117. The capsid polypeptide of any one of embodiments 1 to 116, which comprises a threonine at a position corresponding to Q579 of the VP1 capsid polypeptide of SEQ ID NO:1.
   118. The capsid polypeptide of any one of embodiments 1 to 116, which comprises a valine at a position corresponding to Q579 of the VP1 capsid polypeptide of SEQ ID NO:1.
   119. The capsid polypeptide of any one of embodiments 1 to 118, which comprises a valine at a position corresponding to V580 of the VP1 capsid polypeptide of SEQ ID NO:1.
   120. The capsid polypeptide of any one of embodiments 1 to 119, which comprises an alanine at a position corresponding to A581 of the VP1 capsid polypeptide of SEQ ID NO:1.
   121. The capsid polypeptide of any one of embodiments 1 to 119, which comprises a cysteine at a position corresponding to A581 of the VP1 capsid polypeptide of SEQ ID NO:1.
   122. The capsid polypeptide of any one of embodiments 1 to 119, which comprises an asparagine at a position corresponding to A581 of the VP1 capsid polypeptide of SEQ ID NO:1.
   123. The capsid polypeptide of any one of embodiments 1 to 122, which comprises a threonine at a position corresponding to T582 of the VP1 capsid polypeptide of SEQ ID NO:1.
   124. The capsid polypeptide of any one of embodiments 1 to 122, which comprises an isoleucine at a position corresponding to T582 of the VP1 capsid polypeptide of SEQ ID NO:1.
   125. The capsid polypeptide of any one of embodiments 1 to 122, which comprises a methionine at a position corresponding to T582 of the VP1 capsid polypeptide of SEQ ID NO:1.
   126. The capsid polypeptide of any one of embodiments 1 to 125, which comprises an asparagine at a position corresponding to N583 of the VP1 capsid polypeptide of SEQ ID NO:1.
   127. The capsid polypeptide of any one of embodiments 1 to 126, which comprises a histidine at a position corresponding to H584 of the VP1 capsid polypeptide of SEQ ID NO:1.
   128. The capsid polypeptide of any one of embodiments 1 to 126, which comprises an isoleucine at a position corresponding to H584 of the VP1 capsid polypeptide of SEQ ID NO:1.
   129. The capsid polypeptide of any one of embodiments 1 to 126, which comprises a leucine at a position corresponding to H584 of the VP1 capsid polypeptide of SEQ ID NO:1.
   130. The capsid polypeptide of any one of embodiments 1 to 126, which comprises a methionine at a position corresponding to H584 of the VP1 capsid polypeptide of SEQ ID NO:1.
   131. The capsid polypeptide of any one of embodiments 1 to 126, which comprises an asparagine at a position corresponding to H584 of the VP1 capsid polypeptide of SEQ ID NO:1.
   132. The capsid polypeptide of any one of embodiments 1 to 126, which comprises a glutamine at a position corresponding to H584 of the VP1 capsid polypeptide of SEQ ID NO:1.
   133. The capsid polypeptide of any one of embodiments 1 to 132, which comprises a glutamine at a position corresponding to Q585 of the VP1 capsid polypeptide of SEQ ID NO:1.
   134. The capsid polypeptide of any one of embodiments 1 to 133, which comprises a serine at a position corresponding to S586 of the VP1 capsid polypeptide of SEQ ID NO:1.
   135. The capsid polypeptide of any one of embodiments 1 to 133, which comprises a glutamine at a position corresponding to S586 of the VP1 capsid polypeptide of SEQ ID NO:1.
   136. The capsid polypeptide of any one of embodiments 1 to 135, which comprises an alanine at a position corresponding to A587 of the VP1 capsid polypeptide of SEQ ID NO:1.
   137. The capsid polypeptide of any one of embodiments 1 to 135, which comprises a histidine at a position corresponding to A587 of the VP1 capsid polypeptide of SEQ ID NO:1.
   138. The capsid polypeptide of any one of embodiments 1 to 135, which comprises a serine at a position corresponding to A587 of the VP1 capsid polypeptide of SEQ ID NO:1.
   139. The capsid polypeptide of any one of embodiments 1 to 135, which comprises a threonine at a position corresponding to A587 of the VP1 capsid polypeptide of SEQ ID NO:1.
   140. The capsid polypeptide of any one of embodiments 1 to 139, which comprises a glutamine at a position corresponding to Q588 of the VP1 capsid polypeptide of SEQ ID NO:1.
   141. The capsid polypeptide of any one of embodiments 1 to 139, which comprises a glycine at a position corresponding to Q588 of the VP1 capsid polypeptide of SEQ ID NO:1.
   142. The capsid polypeptide of any one of embodiments 1 to 139, which comprises an asparagine at a position corresponding to Q588 of the VP1 capsid polypeptide of SEQ ID NO:1.
   143. The capsid polypeptide of any one of embodiments 1 to 139, which comprises a threonine at a position corresponding to Q588 of the VP1 capsid polypeptide of SEQ ID NO:1.
   144. The capsid polypeptide of any one of embodiments 1 to 143, which comprises an alanine at a position corresponding to A589 of the VP1 capsid polypeptide of SEQ ID NO:1.
   145. The capsid polypeptide of any one of embodiments 1 to 143, which comprises a serine at a position corresponding to A589 of the VP1 capsid polypeptide of SEQ ID NO:1.
   146. The capsid polypeptide of any one of embodiments 1 to 143, which comprises a threonine at a position corresponding to A589 of the VP1 capsid polypeptide of SEQ ID NO:1.
   147. The capsid polypeptide of any one of embodiments 1 to 146, which comprises a glutamine at a position corresponding to Q590 of the VP1 capsid polypeptide of SEQ ID NO:1.
   148. The capsid polypeptide of any one of embodiments 1 to 147, which comprises an alanine at a position corresponding to A591 of the VP1 capsid polypeptide of SEQ ID NO:1.
   149. The capsid polypeptide of any one of embodiments 1 to 147, which comprises a proline at a position corresponding to A591 of the VP1 capsid polypeptide of SEQ ID NO:1.
   150. The capsid polypeptide of any one of embodiments 1 to 149, which comprises a glycine at a position corresponding to G594 of the VP1 capsid polypeptide of SEQ ID NO:1.
   151. The capsid polypeptide of any one of embodiments 1 to 150, which comprises a glutamine at a position corresponding to Q597 of the VP1 capsid polypeptide of SEQ ID NO:1.
   152. The capsid polypeptide of any one of embodiments 1 to 151, which comprises a glutamine at a position corresponding to Q599 of the VP1 capsid polypeptide of SEQ ID NO:1.
   153. The capsid polypeptide of any one of embodiments 1 to 152, which comprises a glycine at a position corresponding to G600 of the VP1 capsid polypeptide of SEQ ID NO:1.
   154. The capsid polypeptide of any one of embodiments 1 to 153, which comprises a leucine at a position corresponding to L602 of the VP1 capsid polypeptide of SEQ ID NO:1.
   155. The capsid polypeptide of any one of embodiments 1 to 154, which comprises a proline at a position corresponding to P603 of the VP1 capsid polypeptide of SEQ ID NO:1.
   156. The capsid polypeptide of any one of embodiments 1 to 155, which comprises a glycine at a position corresponding to G604 of the VP1 capsid polypeptide of SEQ ID NO:1.
   157. The capsid polypeptide of any one of embodiments 1 to 156, which comprises a methionine at a position corresponding to M605 of the VP1 capsid polypeptide of SEQ ID NO:1.
   158. The capsid polypeptide of any one of embodiments 1 to 157, which comprises a valine at a position corresponding to V606 of the VP1 capsid polypeptide of SEQ ID NO:1.
   159. The capsid polypeptide of any one of embodiments 1 to 158, which comprises a tryptophan at a position corresponding to W607 of the VP1 capsid polypeptide of SEQ ID NO:1.
   160. The capsid polypeptide of any one of embodiments 1 to 159, which comprises a glutamine at a position corresponding to Q608 of the VP1 capsid polypeptide of SEQ ID NO:1.
   161. The capsid polypeptide of any one of embodiments 1 to 160, which comprises an aspartic acid at a position corresponding to D609 of the VP1 capsid polypeptide of SEQ ID NO:1.
   162. The capsid polypeptide of any one of embodiments 1 to 160, which comprises an asparagine at a position corresponding to D609 of the VP1 capsid polypeptide of SEQ ID NO:1.
   163. The capsid polypeptide of any one of embodiments 1 to 162, which comprises an amino acid sequence having at least 70% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   164. The capsid polypeptide of any one of embodiments 1 to 162, which comprises an amino acid sequence having at least 75% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   165. The capsid polypeptide of any one of embodiments 1 to 162, which comprises an amino acid sequence having at least 80% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   166. The capsid polypeptide of any one of embodiments 1 to 162, which comprises an amino acid sequence having at least 85% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   167. The capsid polypeptide of any one of embodiments 1 to 162, which comprises an amino acid sequence having at least 90% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   168. The capsid polypeptide of any one of embodiments 1 to 162, which comprises an amino acid sequence having at least 91% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   169. The capsid polypeptide of any one of embodiments 1 to 162, which comprises an amino acid sequence having at least 92% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   170. The capsid polypeptide of any one of embodiments 1 to 162, which comprises an amino acid sequence having at least 93% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   171. The capsid polypeptide of any one of embodiments 1 to 162, which comprises an amino acid sequence having at least 94% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   172. The capsid polypeptide of any one of embodiments 1 to 162, which comprises an amino acid sequence having at least 95% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   173. The capsid polypeptide of any one of embodiments 1 to 162, which comprises an amino acid sequence having at least 96% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   174. The capsid polypeptide of any one of embodiments 1 to 162, which comprises an amino acid sequence having at least 97% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   175. The capsid polypeptide of any one of embodiments 1 to 162, which comprises an amino acid sequence having at least 98% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   176. The capsid polypeptide of any one of embodiments 1 to 162, which comprises an amino acid sequence having at least 99% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   177. The capsid polypeptide of any one of embodiments 1 to 162, which comprises an amino acid sequence having at least 70% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
   178. The capsid polypeptide of any one of embodiments 1 to 162, which comprises an amino acid sequence having at least 75% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
   179. The capsid polypeptide of any one of embodiments 1 to 162, which comprises an amino acid sequence having at least 80% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
   180. The capsid polypeptide of any one of embodiments 1 to 162, which comprises an amino acid sequence having at least 85% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
   181. The capsid polypeptide of any one of embodiments 1 to 162, which comprises an amino acid sequence having at least 90% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
   182. The capsid polypeptide of any one of embodiments 1 to 162, which comprises an amino acid sequence having at least 91% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
   183. The capsid polypeptide of any one of embodiments 1 to 162, which comprises an amino acid sequence having at least 92% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
   184. The capsid polypeptide of any one of embodiments 1 to 162, which comprises an amino acid sequence having at least 93% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
   185. The capsid polypeptide of any one of embodiments 1 to 162, which comprises an amino acid sequence having at least 94% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
   186. The capsid polypeptide of any one of embodiments 1 to 162, which comprises an amino acid sequence having at least 95% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
   187. The capsid polypeptide of any one of embodiments 1 to 162, which comprises an amino acid sequence having at least 96% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
   188. The capsid polypeptide of any one of embodiments 1 to 162, which comprises an amino acid sequence having at least 97% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
   189. The capsid polypeptide of any one of embodiments 1 to 162, which comprises an amino acid sequence having at least 98% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
   190. The capsid polypeptide of any one of embodiments 1 to 162, which comprises an amino acid sequence having at least 99% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
   191. The capsid polypeptide of any one of embodiments 1 to 162, which comprises an amino acid sequence having at least 70% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
   192. The capsid polypeptide of any one of embodiments 1 to 162, which comprises an amino acid sequence having at least 75% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
   193. The capsid polypeptide of any one of embodiments 1 to 162, which comprises an amino acid sequence having at least 80% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
   194. The capsid polypeptide of any one of embodiments 1 to 162, which comprises an amino acid sequence having at least 85% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
   195. The capsid polypeptide of any one of embodiments 1 to 162, which comprises an amino acid sequence having at least 90% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
   196. The capsid polypeptide of any one of embodiments 1 to 162, which comprises an amino acid sequence having at least 91% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
   197. The capsid polypeptide of any one of embodiments 1 to 162, which comprises an amino acid sequence having at least 92% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
   198. The capsid polypeptide of any one of embodiments 1 to 162, which comprises an amino acid sequence having at least 93% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
   199. The capsid polypeptide of any one of embodiments 1 to 162, which comprises an amino acid sequence having at least 94% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
   200. The capsid polypeptide of any one of embodiments 1 to 162, which comprises an amino acid sequence having at least 95% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
   201. The capsid polypeptide of any one of embodiments 1 to 162, which comprises an amino acid sequence having at least 96% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
   202. The capsid polypeptide of any one of embodiments 1 to 162, which comprises an amino acid sequence having at least 97% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
   203. The capsid polypeptide of any one of embodiments 1 to 162, which comprises an amino acid sequence having at least 98% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
   204. The capsid polypeptide of any one of embodiments 1 to 162, which comprises an amino acid sequence having at least 99% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
   205. The capsid polypeptide of any one of embodiments 1 to 162, which comprises an amino acid sequence having at least 70% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
   206. The capsid polypeptide of any one of embodiments 1 to 162, which comprises an amino acid sequence having at least 75% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
   207. The capsid polypeptide of any one of embodiments 1 to 162, which comprises an amino acid sequence having at least 80% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
   208. The capsid polypeptide of any one of embodiments 1 to 162, which comprises an amino acid sequence having at least 85% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
   209. The capsid polypeptide of any one of embodiments 1 to 162, which comprises an amino acid sequence having at least 90% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
   210. The capsid polypeptide of any one of embodiments 1 to 162, which comprises an amino acid sequence having at least 91% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
   211. The capsid polypeptide of any one of embodiments 1 to 162, which comprises an amino acid sequence having at least 92% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
   212. The capsid polypeptide of any one of embodiments 1 to 162, which comprises an amino acid sequence having at least 93% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
   213. The capsid polypeptide of any one of embodiments 1 to 162, which comprises an amino acid sequence having at least 94% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
   214. The capsid polypeptide of any one of embodiments 1 to 162, which comprises an amino acid sequence having at least 95% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
   215. The capsid polypeptide of any one of embodiments 1 to 162, which comprises an amino acid sequence having at least 96% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
   216. The capsid polypeptide of any one of embodiments 1 to 162, which comprises an amino acid sequence having at least 97% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
   217. The capsid polypeptide of any one of embodiments 1 to 162, which comprises an amino acid sequence having at least 98% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
   218. The capsid polypeptide of any one of embodiments 1 to 162, which comprises an amino acid sequence having at least 99% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
   219. The capsid polypeptide of any one of embodiments 1 to 162, which comprises an amino acid sequence having at least 70% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
   220. The capsid polypeptide of any one of embodiments 1 to 162, which comprises an amino acid sequence having at least 75% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
   221. The capsid polypeptide of any one of embodiments 1 to 162, which comprises an amino acid sequence having at least 80% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
   222. The capsid polypeptide of any one of embodiments 1 to 162, which comprises an amino acid sequence having at least 85% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
   223. The capsid polypeptide of any one of embodiments 1 to 162, which comprises an amino acid sequence having at least 90% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
   224. The capsid polypeptide of any one of embodiments 1 to 162, which comprises an amino acid sequence having at least 91% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
   225. The capsid polypeptide of any one of embodiments 1 to 162, which comprises an amino acid sequence having at least 92% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
   226. The capsid polypeptide of any one of embodiments 1 to 162, which comprises an amino acid sequence having at least 93% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
   227. The capsid polypeptide of any one of embodiments 1 to 162, which comprises an amino acid sequence having at least 94% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
   228. The capsid polypeptide of any one of embodiments 1 to 162, which comprises an amino acid sequence having at least 95% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
   229. The capsid polypeptide of any one of embodiments 1 to 162, which comprises an amino acid sequence having at least 96% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
   230. The capsid polypeptide of any one of embodiments 1 to 162, which comprises an amino acid sequence having at least 97% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
   231. The capsid polypeptide of any one of embodiments 1 to 162, which comprises an amino acid sequence having at least 98% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
   232. The capsid polypeptide of any one of embodiments 1 to 162, which comprises an amino acid sequence having at least 99% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
   233. The capsid polypeptide of any one of embodiments 1 to 162, which comprises an amino acid sequence having at least 70% sequence identity ((a) calculated taking targeting peptide insertions into account or (b) calculated without taking targeting peptide insertions into account) to a VP1 capsid polypeptide of SEQ ID NO: 1 or the VP2 or VP3 portion thereof.
   234. The capsid polypeptide of any one of embodiments 1 to 162, which comprises an amino acid sequence having at least 75% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
   235. The capsid polypeptide of any one of embodiments 1 to 162, which comprises an amino acid sequence having at least 80% sequence identity ((a) calculated taking targeting peptide insertions into account or (b) calculated without taking targeting peptide insertions into account) to a VP1 capsid polypeptide of SEQ ID NO: 1 or the VP2 or VP3 portion thereof.
   236. The capsid polypeptide of any one of embodiments 1 to 162, which comprises an amino acid sequence having at least 85% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
   237. The capsid polypeptide of any one of embodiments 1 to 162, which comprises an amino acid sequence having at least 90% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
   238. The capsid polypeptide of any one of embodiments 1 to 162, which comprises an amino acid sequence having at least 91% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
   239. The capsid polypeptide of any one of embodiments 1 to 162, which comprises an amino acid sequence having at least 92% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
   240. The capsid polypeptide of any one of embodiments 1 to 162, which comprises an amino acid sequence having at least 93% sequence identity ((a) calculated taking targeting peptide insertions into account or (b) calculated without taking targeting peptide insertions into account) to a VP1 capsid polypeptide of SEQ ID NO:1 1 or the VP2 or VP3 portion thereof.
   241. The capsid polypeptide of any one of embodiments 1 to 162, which comprises an amino acid sequence having at least 94% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
   242. The capsid polypeptide of any one of embodiments 1 to 162, which comprises an amino acid sequence having at least 95% sequence identity ((a) calculated taking targeting peptide insertions into account or (b) calculated without taking targeting peptide insertions into account) to a VP1 capsid polypeptide of SEQ ID NO:1 1 or the VP2 or VP3 portion thereof.
   243. The capsid polypeptide of any one of embodiments 1 to 162, which comprises an amino acid sequence having at least 96% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
   244. The capsid polypeptide of any one of embodiments 1 to 162, which comprises an amino acid sequence having at least 97% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
   245. The capsid polypeptide of any one of embodiments 1 to 162, which comprises an amino acid sequence having at least 98% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
   246. The capsid polypeptide of any one of embodiments 1 to 162, which comprises an amino acid sequence having at least 99% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
   247. The capsid polypeptide of any one of embodiments 1 to 246, which comprises an amino acid sequence having at least 70% sequence identity ((a) calculated taking targeting peptide insertions into account or (b) calculated without taking targeting peptide insertions into account) to a VP1 capsid polypeptide of any one of SEQ ID NOs:12-73 or the VP2 or VP3 portion thereof.
   248. The capsid polypeptide of any one of embodiments 1 to 246, which comprises an amino acid sequence having at least 75% sequence identity ((a) calculated taking targeting peptide insertions into account or (b) calculated without taking targeting peptide insertions into account) to a VP1 capsid polypeptide of any one of SEQ ID NOs:12-73 or the VP2 or VP3 portion thereof.
   249. The capsid polypeptide of any one of embodiments 1 to 246, which comprises an amino acid sequence having at least 80% sequence identity ((a) calculated taking targeting peptide insertions into account or (b) calculated without taking targeting peptide insertions into account) to a VP1 capsid polypeptide of any one of SEQ ID NOs:12-73 or the VP2 or VP3 portion thereof.
   250. The capsid polypeptide of any one of embodiments 1 to 246, which comprises an amino acid sequence having at least 85% sequence identity ((a) calculated taking targeting peptide insertions into account or (b) calculated without taking targeting peptide insertions into account) to a VP1 capsid polypeptide of any one of SEQ ID NOs:12-73 or the VP2 or VP3 portion thereof.
   251. The capsid polypeptide of any one of embodiments 1 to 246, which comprises an amino acid sequence having at least 90% sequence identity ((a) calculated taking targeting peptide insertions into account or (b) calculated without taking targeting peptide insertions into account) to a VP1 capsid polypeptide of any one of SEQ ID NOs:12-73 or the VP2 or VP3 portion thereof.
   252. The capsid polypeptide of any one of embodiments 1 to 246, which comprises an amino acid sequence having at least 91% sequence identity ((a) calculated taking targeting peptide insertions into account or (b) calculated without taking targeting peptide insertions into account) to a VP1 capsid polypeptide of any one of SEQ ID NOs:12-73 or the VP2 or VP3 portion thereof.
   253. The capsid polypeptide of any one of embodiments 1 to 246, which comprises an amino acid sequence having at least 92% sequence identity ((a) calculated taking targeting peptide insertions into account or (b) calculated without taking targeting peptide insertions into account) to a VP1 capsid polypeptide of any one of SEQ ID NOs:12-73 or the VP2 or VP3 portion thereof.
   254. The capsid polypeptide of any one of embodiments 1 to 246, which comprises an amino acid sequence having at least 93% sequence identity ((a) calculated taking targeting peptide insertions into account or (b) calculated without taking targeting peptide insertions into account) to a VP1 capsid polypeptide of any one of SEQ ID NOs:12-73 or the VP2 or VP3 portion thereof.
   255. The capsid polypeptide of any one of embodiments 1 to 246, which comprises an amino acid sequence having at least 94% sequence identity ((a) calculated taking targeting peptide insertions into account or (b) calculated without taking targeting peptide insertions into account) to a VP1 capsid polypeptide of any one of SEQ ID NOs:12-73 or the VP2 or VP3 portion thereof.
   256. The capsid polypeptide of any one of embodiments 1 to 246, which comprises an amino acid sequence having at least 95% sequence identity ((a) calculated taking targeting peptide insertions into account or (b) calculated without taking targeting peptide insertions into account) to a VP1 capsid polypeptide of any one of SEQ ID NOs:12-73 or the VP2 or VP3 portion thereof.
   257. The capsid polypeptide of any one of embodiments 1 to 246, which comprises an amino acid sequence having at least 96% sequence identity ((a) calculated taking targeting peptide insertions into account or (b) calculated without taking targeting peptide insertions into account) to a VP1 capsid polypeptide of any one of SEQ ID NOs:12-73 or the VP2 or VP3 portion thereof.
   258. The capsid polypeptide of any one of embodiments 1 to 246, which comprises an amino acid sequence having at least 97% sequence identity ((a) calculated taking targeting peptide insertions into account or (b) calculated without taking targeting peptide insertions into account) to a VP1 capsid polypeptide of any one of SEQ ID NOs:12-73 or the VP2 or VP3 portion thereof.
   259. The capsid polypeptide of any one of embodiments 1 to 246, which comprises an amino acid sequence having at least 98% sequence identity ((a) calculated taking targeting peptide insertions into account or (b) calculated without taking targeting peptide insertions into account) to a VP1 capsid polypeptide of any one of SEQ ID NOs:12-73 or the VP2 or VP3 portion thereof.
   260. The capsid polypeptide of any one of embodiments 1 to 246, which comprises an amino acid sequence having at least 99% sequence identity ((a) calculated taking targeting peptide insertions into account or (b) calculated without taking targeting peptide insertions into account) to a VP1 capsid polypeptide of any one of SEQ ID NOs:12-73 or the VP2 or VP3 portion thereof.
   261. The capsid polypeptide of any one of embodiments 1 to 246, which comprises an amino acid sequence having at least 99.5% sequence identity ((a) calculated taking targeting peptide insertions into account or (b) calculated without taking targeting peptide insertions into account) to a VP1 capsid polypeptide of any one of SEQ ID NOs:12-73 or the VP2 or VP3 portion thereof.
   262. A capsid polypeptide, which is optionally a capsid polypeptide according to any one of embodiments 1 to 261, which comprises the amino acid sequence of a VP1 capsid polypeptide of SEQ ID NO:12 or the VP2 or VP3 portion thereof.
   263. A capsid polypeptide, which is optionally a capsid polypeptide according to any one of embodiments 1 to 261, which comprises the amino acid sequence of a VP1 capsid polypeptide of SEQ ID NO:13 or the VP2 or VP3 portion thereof.
   264. A capsid polypeptide, which is optionally a capsid polypeptide according to any one of embodiments 1 to 261, which comprises the amino acid sequence of a VP1 capsid polypeptide of SEQ ID NO:14 or the VP2 or VP3 portion thereof.
   265. A capsid polypeptide, which is optionally a capsid polypeptide according to any one of embodiments 1 to 261, which comprises the amino acid sequence of a VP1 capsid polypeptide of SEQ ID NO:15 or the VP2 or VP3 portion thereof.
   266. A capsid polypeptide, which is optionally a capsid polypeptide according to any one of embodiments 1 to 261, which comprises the amino acid sequence of a VP1 capsid polypeptide of SEQ ID NO:16 or the VP2 or VP3 portion thereof.
   267. A capsid polypeptide, which is optionally a capsid polypeptide according to any one of embodiments 1 to 261, which comprises the amino acid sequence of a VP1 capsid polypeptide of SEQ ID NO:17 or the VP2 or VP3 portion thereof.
   268. A capsid polypeptide, which is optionally a capsid polypeptide according to any one of embodiments 1 to 261, which comprises the amino acid sequence of a VP1 capsid polypeptide of SEQ ID NO:18 or the VP2 or VP3 portion thereof.
   269. A capsid polypeptide, which is optionally a capsid polypeptide according to any one of embodiments 1 to 261, which comprises the amino acid sequence of a VP1 capsid polypeptide of SEQ ID NO:19 or the VP2 or VP3 portion thereof.
   270. A capsid polypeptide, which is optionally a capsid polypeptide according to any one of embodiments 1 to 261, which comprises the amino acid sequence of a VP1 capsid polypeptide of SEQ ID NO:20 or the VP2 or VP3 portion thereof.
   271. A capsid polypeptide, which is optionally a capsid polypeptide according to any one of embodiments 1 to 261, which comprises the amino acid sequence of a VP1 capsid polypeptide of SEQ ID NO:21 or the VP2 or VP3 portion thereof.
   272. A capsid polypeptide, which is optionally a capsid polypeptide according to any one of embodiments 1 to 261, which comprises the amino acid sequence of a VP1 capsid polypeptide of SEQ ID NO:22 or the VP2 or VP3 portion thereof.
   273. A capsid polypeptide, which is optionally a capsid polypeptide according to any one of embodiments 1 to 261, which comprises the amino acid sequence of a VP1 capsid polypeptide of SEQ ID NO:23 or the VP2 or VP3 portion thereof.
   274. A capsid polypeptide, which is optionally a capsid polypeptide according to any one of embodiments 1 to 261, which comprises the amino acid sequence of a VP1 capsid polypeptide of SEQ ID NO:24 or the VP2 or VP3 portion thereof.
   275. A capsid polypeptide, which is optionally a capsid polypeptide according to any one of embodiments 1 to 261, which comprises the amino acid sequence of a VP1 capsid polypeptide of SEQ ID NO:25 or the VP2 or VP3 portion thereof.
   276. A capsid polypeptide, which is optionally a capsid polypeptide according to any one of embodiments 1 to 261, which comprises the amino acid sequence of a VP1 capsid polypeptide of SEQ ID NO:26 or the VP2 or VP3 portion thereof.
   277. A capsid polypeptide, which is optionally a capsid polypeptide according to any one of embodiments 1 to 261, which comprises the amino acid sequence of a VP1 capsid polypeptide of SEQ ID NO:27 or the VP2 or VP3 portion thereof.
   278. A capsid polypeptide, which is optionally a capsid polypeptide according to any one of embodiments 1 to 261, which comprises the amino acid sequence of a VP1 capsid polypeptide of SEQ ID NO:28 or the VP2 or VP3 portion thereof.
   279. A capsid polypeptide, which is optionally a capsid polypeptide according to any one of embodiments 1 to 261, which comprises the amino acid sequence of a VP1 capsid polypeptide of SEQ ID NO:29 or the VP2 or VP3 portion thereof.
   280. A capsid polypeptide, which is optionally a capsid polypeptide according to any one of embodiments 1 to 261, which comprises the amino acid sequence of a VP1 capsid polypeptide of SEQ ID NO:30 or the VP2 or VP3 portion thereof.
   281. A capsid polypeptide, which is optionally a capsid polypeptide according to any one of embodiments 1 to 261, which comprises the amino acid sequence of a VP1 capsid polypeptide of SEQ ID NO:31 or the VP2 or VP3 portion thereof.
   282. A capsid polypeptide, which is optionally a capsid polypeptide according to any one of embodiments 1 to 261, which comprises the amino acid sequence of a VP1 capsid polypeptide of SEQ ID NO:32 or the VP2 or VP3 portion thereof.
   283. A capsid polypeptide, which is optionally a capsid polypeptide according to any one of embodiments 1 to 261, which comprises the amino acid sequence of a VP1 capsid polypeptide of SEQ ID NO:33 or the VP2 or VP3 portion thereof.
   284. A capsid polypeptide, which is optionally a capsid polypeptide according to any one of embodiments 1 to 261, which comprises the amino acid sequence of a VP1 capsid polypeptide of SEQ ID NO:34 or the VP2 or VP3 portion thereof.
   285. A capsid polypeptide, which is optionally a capsid polypeptide according to any one of embodiments 1 to 261, which comprises the amino acid sequence of a VP1 capsid polypeptide of SEQ ID NO:35 or the VP2 or VP3 portion thereof.
   286. A capsid polypeptide, which is optionally a capsid polypeptide according to any one of embodiments 1 to 261, which comprises the amino acid sequence of a VP1 capsid polypeptide of SEQ ID NO:36 or the VP2 or VP3 portion thereof.
   287. A capsid polypeptide, which is optionally a capsid polypeptide according to any one of embodiments 1 to 261, which comprises the amino acid sequence of a VP1 capsid polypeptide of SEQ ID NO:37 or the VP2 or VP3 portion thereof.
   288. A capsid polypeptide, which is optionally a capsid polypeptide according to any one of embodiments 1 to 261, which comprises the amino acid sequence of a VP1 capsid polypeptide of SEQ ID NO:38 or the VP2 or VP3 portion thereof.
   289. A capsid polypeptide, which is optionally a capsid polypeptide according to any one of embodiments 1 to 261, which comprises the amino acid sequence of a VP1 capsid polypeptide of SEQ ID NO:39 or the VP2 or VP3 portion thereof.
   290. A capsid polypeptide, which is optionally a capsid polypeptide according to any one of embodiments 1 to 261, which comprises the amino acid sequence of a VP1 capsid polypeptide of SEQ ID NO:40 or the VP2 or VP3 portion thereof.
   291. A capsid polypeptide, which is optionally a capsid polypeptide according to any one of embodiments 1 to 261, which comprises the amino acid sequence of a VP1 capsid polypeptide of SEQ ID NO:41 or the VP2 or VP3 portion thereof.
   292. A capsid polypeptide, which is optionally a capsid polypeptide according to any one of embodiments 1 to 261, which comprises the amino acid sequence of a VP1 capsid polypeptide of SEQ ID NO:42 or the VP2 or VP3 portion thereof.
   293. A capsid polypeptide, which is optionally a capsid polypeptide according to any one of embodiments 1 to 261, which comprises the amino acid sequence of a VP1 capsid polypeptide of SEQ ID NO:43 or the VP2 or VP3 portion thereof.
   294. A capsid polypeptide, which is optionally a capsid polypeptide according to any one of embodiments 1 to 261, which comprises the amino acid sequence of a VP1 capsid polypeptide of SEQ ID NO:44 or the VP2 or VP3 portion thereof.
   295. A capsid polypeptide, which is optionally a capsid polypeptide according to any one of embodiments 1 to 261, which comprises the amino acid sequence of a VP1 capsid polypeptide of SEQ ID NO:45 or the VP2 or VP3 portion thereof.
   296. A capsid polypeptide, which is optionally a capsid polypeptide according to any one of embodiments 1 to 261, which comprises the amino acid sequence of a VP1 capsid polypeptide of SEQ ID NO:46 or the VP2 or VP3 portion thereof.
   297. A capsid polypeptide, which is optionally a capsid polypeptide according to any one of embodiments 1 to 261, which comprises the amino acid sequence of a VP1 capsid polypeptide of SEQ ID NO:47 or the VP2 or VP3 portion thereof.
   298. A capsid polypeptide, which is optionally a capsid polypeptide according to any one of embodiments 1 to 261, which comprises the amino acid sequence of a VP1 capsid polypeptide of SEQ ID NO:48 or the VP2 or VP3 portion thereof.
   299. A capsid polypeptide, which is optionally a capsid polypeptide according to any one of embodiments 1 to 261, which comprises the amino acid sequence of a VP1 capsid polypeptide of SEQ ID NO:49 or the VP2 or VP3 portion thereof.
   300. A capsid polypeptide, which is optionally a capsid polypeptide according to any one of embodiments 1 to 261, which comprises the amino acid sequence of a VP1 capsid polypeptide of SEQ ID NO:50 or the VP2 or VP3 portion thereof.
   301. A capsid polypeptide, which is optionally a capsid polypeptide according to any one of embodiments 1 to 261, which comprises the amino acid sequence of a VP1 capsid polypeptide of SEQ ID NO:51 or the VP2 or VP3 portion thereof.
   302. A capsid polypeptide, which is optionally a capsid polypeptide according to any one of embodiments 1 to 261, which comprises the amino acid sequence of a VP1 capsid polypeptide of SEQ ID NO:52 or the VP2 or VP3 portion thereof.
   303. A capsid polypeptide, which is optionally a capsid polypeptide according to any one of embodiments 1 to 261, which comprises the amino acid sequence of a VP1 capsid polypeptide of SEQ ID NO:53 or the VP2 or VP3 portion thereof.
   304. A capsid polypeptide, which is optionally a capsid polypeptide according to any one of embodiments 1 to 261, which comprises the amino acid sequence of a VP1 capsid polypeptide of SEQ ID NO:54 or the VP2 or VP3 portion thereof.
   305. A capsid polypeptide, which is optionally a capsid polypeptide according to any one of embodiments 1 to 261, which comprises the amino acid sequence of a VP1 capsid polypeptide of SEQ ID NO:55 or the VP2 or VP3 portion thereof.
   306. A capsid polypeptide, which is optionally a capsid polypeptide according to any one of embodiments 1 to 261, which comprises the amino acid sequence of a VP1 capsid polypeptide of SEQ ID NO:56 or the VP2 or VP3 portion thereof.
   307. A capsid polypeptide, which is optionally a capsid polypeptide according to any one of embodiments 1 to 261, which comprises the amino acid sequence of a VP1 capsid polypeptide of SEQ ID NO:57 or the VP2 or VP3 portion thereof.
   308. A capsid polypeptide, which is optionally a capsid polypeptide according to any one of embodiments 1 to 261, which comprises the amino acid sequence of a VP1 capsid polypeptide of SEQ ID NO:58 or the VP2 or VP3 portion thereof.
   309. A capsid polypeptide, which is optionally a capsid polypeptide according to any one of embodiments 1 to 261, which comprises the amino acid sequence of a VP1 capsid polypeptide of SEQ ID NO:59 or the VP2 or VP3 portion thereof.
   310. A capsid polypeptide, which is optionally a capsid polypeptide according to any one of embodiments 1 to 261, which comprises the amino acid sequence of a VP1 capsid polypeptide of SEQ ID NO:60 or the VP2 or VP3 portion thereof.
   311. A capsid polypeptide, which is optionally a capsid polypeptide according to any one of embodiments 1 to 261, which comprises the amino acid sequence of a VP1 capsid polypeptide of SEQ ID NO:61 or the VP2 or VP3 portion thereof.
   312. A capsid polypeptide, which is optionally a capsid polypeptide according to any one of embodiments 1 to 261, which comprises the amino acid sequence of a VP1 capsid polypeptide of SEQ ID NO:62 or the VP2 or VP3 portion thereof.
   313. A capsid polypeptide, which is optionally a capsid polypeptide according to any one of embodiments 1 to 261, which comprises the amino acid sequence of a VP1 capsid polypeptide of SEQ ID NO:63 or the VP2 or VP3 portion thereof.
   314. A capsid polypeptide, which is optionally a capsid polypeptide according to any one of embodiments 1 to 261, which comprises the amino acid sequence of a VP1 capsid polypeptide of SEQ ID NO:64 or the VP2 or VP3 portion thereof.
   315. A capsid polypeptide, which is optionally a capsid polypeptide according to any one of embodiments 1 to 261, which comprises the amino acid sequence of a VP1 capsid polypeptide of SEQ ID NO:65 or the VP2 or VP3 portion thereof.
   316. A capsid polypeptide, which is optionally a capsid polypeptide according to any one of embodiments 1 to 261, which comprises the amino acid sequence of a VP1 capsid polypeptide of SEQ ID NO:66 or the VP2 or VP3 portion thereof.
   317. A capsid polypeptide, which is optionally a capsid polypeptide according to any one of embodiments 1 to 261, which comprises the amino acid sequence of a VP1 capsid polypeptide of SEQ ID NO:67 or the VP2 or VP3 portion thereof.
   318. A capsid polypeptide, which is optionally a capsid polypeptide according to any one of embodiments 1 to 261, which comprises the amino acid sequence of a VP1 capsid polypeptide of SEQ ID NO:68 or the VP2 or VP3 portion thereof.
   319. A capsid polypeptide, which is optionally a capsid polypeptide according to any one of embodiments 1 to 261, which comprises the amino acid sequence of a VP1 capsid polypeptide of SEQ ID NO:69 or the VP2 or VP3 portion thereof.
   320. A capsid polypeptide, which is optionally a capsid polypeptide according to any one of embodiments 1 to 261, which comprises the amino acid sequence of a VP1 capsid polypeptide of SEQ ID NO:70 or the VP2 or VP3 portion thereof.
   321. A capsid polypeptide, which is optionally a capsid polypeptide according to any one of embodiments 1 to 261, which comprises the amino acid sequence of a VP1 capsid polypeptide of SEQ ID NO:71 or the VP2 or VP3 portion thereof.
   322. A capsid polypeptide, which is optionally a capsid polypeptide according to any one of embodiments 1 to 261, which comprises the amino acid sequence of a VP1 capsid polypeptide of SEQ ID NO:72 or the VP2 or VP3 portion thereof.
   323. A capsid polypeptide, which is optionally a capsid polypeptide according to any one of embodiments 1 to 261, which comprises the amino acid sequence of a VP1 capsid polypeptide of SEQ ID NO:73 or the VP2 or VP3 portion thereof.
   324. A capsid polypeptide which:
(a) comprises at least 70%, at least 80%, or at least 90% (and preferably at least 70%) of the mutations in the mutation set of VAR-1; and
(b) comprises an amino acid sequence with an edit distance of 15 or less to the amino acid sequence of SEQ ID NO:12 (or to the VP2 or VP3 portion thereof).
   325. A capsid polypeptide which:
(a) comprises at least 70%, at least 80%, or at least 90% (and preferably at least 70%) of the mutations in the mutation set of VAR-2; and
(b) comprises an amino acid sequence with an edit distance of 15 or less to the amino acid sequence of SEQ ID NO:13 (or to the VP2 or VP3 portion thereof).
   326. A capsid polypeptide which:
(a) comprises at least 70%, at least 80%, or at least 90% (and preferably at least 70%) of the mutations in the mutation set of VAR-3; and
(b) comprises an amino acid sequence with an edit distance of 15 or less to the amino acid sequence of SEQ ID NO:14 (or to the VP2 or VP3 portion thereof).
   327. A capsid polypeptide which:
(a) comprises at least 70%, at least 80%, or at least 90% (and preferably at least 70%) of the mutations in the mutation set of VAR-4; and
(b) comprises an amino acid sequence with an edit distance of 15 or less to the amino acid sequence of SEQ ID NO:15 (or to the VP2 or VP3 portion thereof).
   328. A capsid polypeptide which:
(a) comprises at least 70%, at least 80%, or at least 90% (and preferably at least 70%) of the mutations in the mutation set of VAR-5; and
(b) comprises an amino acid sequence with an edit distance of 15 or less to the amino acid sequence of SEQ ID NO:16 (or to the VP2 or VP3 portion thereof).
   329. A capsid polypeptide which:
(a) comprises at least 70%, at least 80%, or at least 90% (and preferably at least 70%) of the mutations in the mutation set of VAR-6; and
(b) comprises an amino acid sequence with an edit distance of 15 or less to the amino acid sequence of SEQ ID NO:17 (or to the VP2 or VP3 portion thereof).
   330. A capsid polypeptide which:
(a) comprises at least 70%, at least 80%, or at least 90% (and preferably at least 70%) of the mutations in the mutation set of VAR-7; and
(b) comprises an amino acid sequence with an edit distance of 15 or less to the amino acid sequence of SEQ ID NO:18 (or to the VP2 or VP3 portion thereof).
   331. A capsid polypeptide which:
(a) comprises at least 70%, at least 80%, or at least 90% (and preferably at least 70%) of the mutations in the mutation set of VAR-8; and
(b) comprises an amino acid sequence with an edit distance of 15 or less to the amino acid sequence of SEQ ID NO:19 (or to the VP2 or VP3 portion thereof).
   332. A capsid polypeptide which:
(a) comprises at least 70%, at least 80%, or at least 90% (and preferably at least 70%) of the mutations in the mutation set of VAR-9; and
(b) comprises an amino acid sequence with an edit distance of 15 or less to the amino acid sequence of SEQ ID NO:20 (or to the VP2 or VP3 portion thereof).
   333. A capsid polypeptide which:
(a) comprises at least 70%, at least 80%, or at least 90% (and preferably at least 70%) of the mutations in the mutation set of VAR-10; and
(b) comprises an amino acid sequence with an edit distance of 15 or less to the amino acid sequence of SEQ ID NO:21 (or to the VP2 or VP3 portion thereof).
   334. A capsid polypeptide which:
(a) comprises at least 70%, at least 80%, or at least 90% (and preferably at least 70%) of the mutations in the mutation set of VAR-11; and
(b) comprises an amino acid sequence with an edit distance of 15 or less to the amino acid sequence of SEQ ID NO:22 (or to the VP2 or VP3 portion thereof).
   335. A capsid polypeptide which:
(a) comprises at least 70%, at least 80%, or at least 90% (and preferably at least 70%) of the mutations in the mutation set of VAR-12, and
(b) comprises an amino acid sequence with an edit distance of 15 or less to the amino acid sequence of SEQ ID NO:23 (or to the VP2 or VP3 portion thereof).
   336. A capsid polypeptide which:
(a) comprises at least 70%, at least 80%, or at least 90% (and preferably at least 70%) of the mutations in the mutation set of VAR-13; and
(b) comprises an amino acid sequence with an edit distance of 15 or less to the amino acid sequence of SEQ ID NO:24 (or to the VP2 or VP3 portion thereof).
   337. A capsid polypeptide which:
(a) comprises at least 70%, at least 80%, or at least 90% (and preferably at least 70%) of the mutations in the mutation set of VAR-14; and
(b) comprises an amino acid sequence with an edit distance of 15 or less to the amino acid sequence of SEQ ID NO:25 (or to the VP2 or VP3 portion thereof).
   338. A capsid polypeptide which:
(a) comprises at least 70%, at least 80%, or at least 90% (and preferably at least 70%) of the mutations in the mutation set of VAR-15; and
(b) comprises an amino acid sequence with an edit distance of 15 or less to the amino acid sequence of SEQ ID NO:26 (or to the VP2 or VP3 portion thereof).
   339. A capsid polypeptide which:
(a) comprises at least 70%, at least 80%, or at least 90% (and preferably at least 70%) of the mutations in the mutation set of VAR-16; and
(b) comprises an amino acid sequence with an edit distance of 15 or less to the amino acid sequence of SEQ ID NO:27 (or to the VP2 or VP3 portion thereof).
   340. A capsid polypeptide which:
(a) comprises at least 70%, at least 80%, or at least 90% (and preferably at least 70%) of the mutations in the mutation set of VAR-17; and
(b) comprises an amino acid sequence with an edit distance of 15 or less to the amino acid sequence of SEQ ID NO:28 (or to the VP2 or VP3 portion thereof).
   341. A capsid polypeptide which:
(a) comprises at least 70%, at least 80%, or at least 90% (and preferably at least 70%) of the mutations in the mutation set of VAR-18; and
(b) comprises an amino acid sequence with an edit distance of 15 or less to the amino acid sequence of SEQ ID NO:29 (or to the VP2 or VP3 portion thereof).
   342. A capsid polypeptide which:
(a) comprises at least 70%, at least 80%, or at least 90% (and preferably at least 70%) of the mutations in the mutation set of VAR-19, and
(b) comprises an amino acid sequence with an edit distance of 15 or less to the amino acid sequence of SEQ ID NO:30 (or to the VP2 or VP3 portion thereof).
   343. A capsid polypeptide which:
(a) comprises at least 70%, at least 80%, or at least 90% (and preferably at least 70%) of the mutations in the mutation set of VAR-20; and
(b) comprises an amino acid sequence with an edit distance of 15 or less to the amino acid sequence of SEQ ID NO:31 (or to the VP2 or VP3 portion thereof).
   344. A capsid polypeptide which:
(a) comprises at least 70%, at least 80%, or at least 90% (and preferably at least 70%) of the mutations in the mutation set of VAR-21; and
(b) comprises an amino acid sequence with an edit distance of 15 or less to the amino acid sequence of SEQ ID NO:32 (or to the VP2 or VP3 portion thereof).
   345. A capsid polypeptide which:
(a) comprises at least 70%, at least 80%, or at least 90% (and preferably at least 70%) of the mutations in the mutation set of VAR-22; and
(b) comprises an amino acid sequence with an edit distance of 15 or less to the amino acid sequence of SEQ ID NO:33 (or to the VP2 or VP3 portion thereof).
   346. A capsid polypeptide which:
(a) comprises at least 70%, at least 80%, or at least 90% (and preferably at least 70%) of the mutations in the mutation set of VAR-23; and
(b) comprises an amino acid sequence with an edit distance of 15 or less to the amino acid sequence of SEQ ID NO:34 (or to the VP2 or VP3 portion thereof).
   347. A capsid polypeptide which:
(a) comprises at least 70%, at least 80%, or at least 90% (and preferably at least 70%) of the mutations in the mutation set of VAR-24; and
(b) comprises an amino acid sequence with an edit distance of 15 or less to the amino acid sequence of SEQ ID NO:35 (or to the VP2 or VP3 portion thereof).
   348. A capsid polypeptide which:
(a) comprises at least 70%, at least 80%, or at least 90% (and preferably at least 70%) of the mutations in the mutation set of VAR-25; and
(b) comprises an amino acid sequence with an edit distance of 15 or less to the amino acid sequence of SEQ ID NO:36 (or to the VP2 or VP3 portion thereof).
   349. A capsid polypeptide which:
(a) comprises at least 70%, at least 80%, or at least 90% (and preferably at least 70%) of the mutations in the mutation set of VAR-26, and
(b) comprises an amino acid sequence with an edit distance of 15 or less to the amino acid sequence of SEQ ID NO:37 (or to the VP2 or VP3 portion thereof).
   350. A capsid polypeptide which:
(a) comprises at least 70%, at least 80%, or at least 90% (and preferably at least 70%) of the mutations in the mutation set of VAR-27; and
(b) comprises an amino acid sequence with an edit distance of 15 or less to the amino acid sequence of SEQ ID NO:38 (or to the VP2 or VP3 portion thereof).
   351. A capsid polypeptide which:
(a) comprises at least 70%, at least 80%, or at least 90% (and preferably at least 70%) of the mutations in the mutation set of VAR-28; and
(b) comprises an amino acid sequence with an edit distance of 15 or less to the amino acid sequence of SEQ ID NO:39 (or to the VP2 or VP3 portion thereof).
   352. A capsid polypeptide which:
(a) comprises at least 70%, at least 80%, or at least 90% (and preferably at least 70%) of the mutations in the mutation set of VAR-29; and
(b) comprises an amino acid sequence with an edit distance of 15 or less to the amino acid sequence of SEQ ID NO:40 (or to the VP2 or VP3 portion thereof).
   353. A capsid polypeptide which:
(a) comprises at least 70%, at least 80%, or at least 90% (and preferably at least 70%) of the mutations in the mutation set of VAR-30; and
(b) comprises an amino acid sequence with an edit distance of 15 or less to the amino acid sequence of SEQ ID NO:41 (or to the VP2 or VP3 portion thereof).
   354. A capsid polypeptide which:
(a) comprises at least 70%, at least 80%, or at least 90% (and preferably at least 70%) of the mutations in the mutation set of VAR-31; and
(b) comprises an amino acid sequence with an edit distance of 15 or less to the amino acid sequence of SEQ ID NO:42 (or to the VP2 or VP3 portion thereof).
   355. A capsid polypeptide which:
(a) comprises at least 70%, at least 80%, or at least 90% (and preferably at least 70%) of the mutations in the mutation set of VAR-32; and
(b) comprises an amino acid sequence with an edit distance of 15 or less to the amino acid sequence of SEQ ID NO:43 (or to the VP2 or VP3 portion thereof).
   356. A capsid polypeptide which:
(a) comprises at least 70%, at least 80%, or at least 90% (and preferably at least 70%) of the mutations in the mutation set of VAR-33; and
(b) comprises an amino acid sequence with an edit distance of 15 or less to the amino acid sequence of SEQ ID NO:14 (or to the VP2 or VP3 portion thereof).
   357. A capsid polypeptide which:
(a) comprises at least 70%, at least 80%, or at least 90% (and preferably at least 70%) of the mutations in the mutation set of VAR-4; and
(b) comprises an amino acid sequence with an edit distance of 15 or less to the amino acid sequence of SEQ ID NO:45 (or to the VP2 or VP3 portion thereof).
   358. A capsid polypeptide which:
(a) comprises at least 70%, at least 80%, or at least 90% (and preferably at least 70%) of the mutations in the mutation set of VAR-35; and
(b) comprises an amino acid sequence with an edit distance of 15 or less to the amino acid sequence of SEQ ID NO:46 (or to the VP2 or VP3 portion thereof).
   359. A capsid polypeptide which:
(a) comprises at least 70%, at least 80%, or at least 90% (and preferably at least 70%) of the mutations in the mutation set of VAR-36 (or to the VP2 or VP3 portion thereof); and (b) comprises an amino acid sequence with an edit distance of 15 or less to the amino acid sequence of SEQ ID NO:47 (or to the VP2 or VP3 portion thereof).
   360. A capsid polypeptide which:
(a) comprises at least 70%, at least 80%, or at least 90% (and preferably at least 70%) of the mutations in the mutation set of VAR-37; and
(b) comprises an amino acid sequence with an edit distance of 15 or less to the amino acid sequence of SEQ ID NO:48 (or to the VP2 or VP3 portion thereof).
   361. A capsid polypeptide which:
(a) comprises at least 70%, at least 80%, or at least 90% (and preferably at least 70%) of the mutations in the mutation set of VAR-38; and
(b) comprises an amino acid sequence with an edit distance of 15 or less to the amino acid sequence of SEQ ID NO:49 (or to the VP2 or VP3 portion thereof).
   362. A capsid polypeptide which:
(a) comprises at least 70%, at least 80%, or at least 90% (and preferably at least 70%) of the mutations in the mutation set of VAR-39; and
(b) comprises an amino acid sequence with an edit distance of 15 or less to the amino acid sequence of SEQ ID NO:50 (or to the VP2 or VP3 portion thereof).
   363. A capsid polypeptide which:
(a) comprises at least 70%, at least 80%, or at least 90% (and preferably at least 70%) of the mutations in the mutation set of VAR-40; and
(b) comprises an amino acid sequence with an edit distance of 15 or less to the amino acid sequence of SEQ ID NO:51 (or to the VP2 or VP3 portion thereof).
   364. A capsid polypeptide which:
(a) comprises at least 70%, at least 80%, or at least 90% (and preferably at least 70%) of the mutations in the mutation set of VAR-41; and
(b) comprises an amino acid sequence with an edit distance of 15 or less to the amino acid sequence of SEQ ID NO:52 (or to the VP2 or VP3 portion thereof).
   365. A capsid polypeptide which:
(a) comprises at least 70%, at least 80%, or at least 90% (and preferably at least 70%) of the mutations in the mutation set of VAR-42; and
(b) comprises an amino acid sequence with an edit distance of 15 or less to the amino acid sequence of SEQ ID NO:53 (or to the VP2 or VP3 portion thereof).
   366. A capsid polypeptide which:
(a) comprises at least 70%, at least 80%, or at least 90% (and preferably at least 70%) of the mutations in the mutation set of VAR-43; and
(b) comprises an amino acid sequence with an edit distance of 15 or less to the amino acid sequence of SEQ ID NO:54 (or to the VP2 or VP3 portion thereof).
   367. A capsid polypeptide which:
(a) comprises at least 70%, at least 80%, or at least 90% (and preferably at least 70%) of the mutations in the mutation set of VAR-44; and
(b) comprises an amino acid sequence with an edit distance of 15 or less to the amino acid sequence of SEQ ID NO:55 (or to the VP2 or VP3 portion thereof).
   368. A capsid polypeptide which:
(a) comprises at least 70%, at least 80%, or at least 90% (and preferably at least 70%) of the mutations in the mutation set of VAR-45; and
(b) comprises an amino acid sequence with an edit distance of 15 or less to the amino acid sequence of SEQ ID NO:56 (or to the VP2 or VP3 portion thereof).
   369. A capsid polypeptide which:
(a) comprises at least 70%, at least 80%, or at least 90% (and preferably at least 70%) of the mutations in the mutation set of VAR-46; and
(b) comprises an amino acid sequence with an edit distance of 15 or less to the amino acid sequence of SEQ ID NO:57 (or to the VP2 or VP3 portion thereof).
   370. A capsid polypeptide which:
(a) comprises at least 70%, at least 80%, or at least 90% (and preferably at least 70%) of the mutations in the mutation set of VAR-47; and
(b) comprises an amino acid sequence with an edit distance of 15 or less to the amino acid sequence of SEQ ID NO:58 (or to the VP2 or VP3 portion thereof).
   371. A capsid polypeptide which:
(a) comprises at least 70%, at least 80%, or at least 90% (and preferably at least 70%) of the mutations in the mutation set of VAR-48; and
(b) comprises an amino acid sequence with an edit distance of 15 or less to the amino acid sequence of SEQ ID NO:59 (or to the VP2 or VP3 portion thereof).
   372. A capsid polypeptide which:
(a) comprises at least 70%, at least 80%, or at least 90% (and preferably at least 70%) of the mutations in the mutation set of VAR-49; and
(b) comprises an amino acid sequence with an edit distance of 15 or less to the amino acid sequence of SEQ ID NO:60 (or to the VP2 or VP3 portion thereof).
   373. A capsid polypeptide which:
(a) comprises at least 70%, at least 80%, or at least 90% (and preferably at least 70%) of the mutations in the mutation set of VAR-50; and
(b) comprises an amino acid sequence with an edit distance of 15 or less to the amino acid sequence of SEQ ID NO:61 (or to the VP2 or VP3 portion thereof).
   374. A capsid polypeptide which:
(a) comprises at least 70%, at least 80%, or at least 90% (and preferably at least 70%) of the mutations in the mutation set of VAR-51, and
(b) comprises an amino acid sequence with an edit distance of 15 or less to the amino acid sequence of SEQ ID NO:62 (or to the VP2 or VP3 portion thereof).
   375. A capsid polypeptide which:
(a) comprises at least 70%, at least 80%, or at least 90% (and preferably at least 70%) of the mutations in the mutation set of VAR-52; and
(b) comprises an amino acid sequence with an edit distance of 15 or less to the amino acid sequence of SEQ ID NO:63 (or to the VP2 or VP3 portion thereof).
   376. A capsid polypeptide which:
(a) comprises at least 70%, at least 80%, or at least 90% (and preferably at least 70%) of the mutations in the mutation set of VAR-53; and
(b) comprises an amino acid sequence with an edit distance of 15 or less to the amino acid sequence of SEQ ID NO:64 (or to the VP2 or VP3 portion thereof).
   377. A capsid polypeptide which:
(a) comprises at least 70%, at least 80%, or at least 90% (and preferably at least 70%) of the mutations in the mutation set of VAR-54; and
(b) comprises an amino acid sequence with an edit distance of 15 or less to the amino acid sequence of SEQ ID NO:55 (or to the VP2 or VP3 portion thereof).
   378. A capsid polypeptide which:
(a) comprises at least 70%, at least 80%, or at least 90% (and preferably at least 70%) of the mutations in the mutation set of VAR-55; and
(b) comprises an amino acid sequence with an edit distance of 15 or less to the amino acid sequence of SEQ ID NO:56 (or to the VP2 or VP3 portion thereof).
   379. A capsid polypeptide which:
(a) comprises at least 70%, at least 80%, or at least 90% (and preferably at least 70%) of the mutations in the mutation set of VAR-56; and
(b) comprises an amino acid sequence with an edit distance of 15 or less to the amino acid sequence of SEQ ID NO:70 (or to the VP2 or VP3 portion thereof).
   380. A capsid polypeptide which:
(a) comprises at least 70%, at least 80%, or at least 90% (and preferably at least 70%) of the mutations in the mutation set of VAR-57; and
(b) comprises an amino acid sequence with an edit distance of 15 or less to the amino acid sequence of SEQ ID NO:69 (or to the VP2 or VP3 portion thereof).
   381. A capsid polypeptide which:
(a) comprises at least 70%, at least 80%, or at least 90% (and preferably at least 70%) of the mutations in the mutation set of VAR-58, and
(b) comprises an amino acid sequence with an edit distance of 15 or less to the amino acid sequence of SEQ ID NO:71 (or to the VP2 or VP3 portion thereof).
   382. A capsid polypeptide which:
(a) comprises at least 70%, at least 80%, or at least 90% (and preferably at least 70%) of the mutations in the mutation set of VAR-59; and
(b) comprises an amino acid sequence with an edit distance of 15 or less to the amino acid sequence of SEQ ID NO:72 (or to the VP2 or VP3 portion thereof).
   383. A capsid polypeptide which:
(a) comprises at least 70%, at least 80%, or at least 90% (and preferably at least 70%) of the mutations in the mutation set of VAR-60; and
(b) comprises an amino acid sequence with an edit distance of 15 or less to the amino acid sequence of SEQ ID NO:67 (or to the VP2 or VP3 portion thereof).
   384. A capsid polypeptide which:
(a) comprises at least 70%, at least 80%, or at least 90% (and preferably at least 70%) of the mutations in the mutation set of VAR-61; and
(b) comprises an amino acid sequence with an edit distance of 15 or less to the amino acid sequence of SEQ ID NO:68 (or to the VP2 or VP3 portion thereof).
   385. A capsid polypeptide which:
(a) comprises at least 70%, at least 80%, or at least 90% (and preferably at least 70%) of the mutations in the mutation set of VAR-62, and
(b) comprises an amino acid sequence with an edit distance of 15 or less to the amino acid sequence of SEQ ID NO:73 (or to the VP2 or VP3 portion thereof).
   386. A capsid polypeptide comprising 5, 6 or all 7 mutations of the mutation set of VAR-1 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   387. A capsid polypeptide comprising 6, 7 or all 8 mutations of the mutation set of VAR-2 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   388. A capsid polypeptide comprising 5, 6 or all 7 mutations of the mutation set of VAR-3 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   389. A capsid polypeptide comprising 5, 6 or all 7 mutations of the mutation set of VAR-4 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   390. A capsid polypeptide comprising 6, 7 or all 8 mutations of the mutation set of VAR-5 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   391. A capsid polypeptide comprising 5, 6 or all 7 mutations of the mutation set of VAR-6 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   392. A capsid polypeptide comprising 5, 6 or all 7 mutations of the mutation set of VAR-7 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   393. A capsid polypeptide comprising 6, 7 or all 8 mutations of the mutation set of VAR-8 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   394. A capsid polypeptide comprising 5, 6 or all 7 mutations of the mutation set of VAR-9 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   395. A capsid polypeptide comprising 7, 8 or all 9 mutations of the mutation set of VAR-10 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   396. A capsid polypeptide comprising 4, 5 or all 6 mutations of the mutation set of VAR-11 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   397. A capsid polypeptide comprising 5, 6 or all 7 mutations of the mutation set of VAR-12 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   398. A capsid polypeptide comprising 5, 6 or all 7 mutations of the mutation set of VAR-13 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   399. A capsid polypeptide comprising 7, 8 or all 9 mutations of the mutation set of VAR-14 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   400. A capsid polypeptide comprising 6, 7 or all 8 mutations of the mutation set of VAR-15 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   401. A capsid polypeptide comprising 4, 5 or all 6 mutations of the mutation set of VAR-16 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   402. A capsid polypeptide comprising 4, 5 or all 6 mutations of the mutation set of VAR-17 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   403. A capsid polypeptide comprising 5, 6 or all 7 mutations of the mutation set of VAR-18 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   404. A capsid polypeptide comprising 5, 6 or all 7 mutations of the mutation set of VAR-19 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   405. A capsid polypeptide comprising 5, 6 or all 7 mutations of the mutation set of VAR-20 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   406. A capsid polypeptide comprising 7, 8 or all 9 mutations of the mutation set of VAR-21 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   407. A capsid polypeptide comprising 5, 6 or all 7 mutations of the mutation set of VAR-22 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   408. A capsid polypeptide comprising 7, 8 or all 9 mutations of the mutation set of VAR-23 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   409. A capsid polypeptide comprising 6, 7 or all 8 mutations of the mutation set of VAR-24 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   410. A capsid polypeptide comprising 5, 6 or all 7 mutations of the mutation set of VAR-25 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   411. A capsid polypeptide comprising 6, 7 or all 8 mutations of the mutation set of VAR-26 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   412. A capsid polypeptide comprising 6, 7 or all 8 mutations of the mutation set of VAR-27 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   413. A capsid polypeptide comprising 5, 6 or all 7 mutations of the mutation set of VAR-28 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   414. A capsid polypeptide comprising 8, 9 or all 10 mutations of the mutation set of VAR-29 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   415. A capsid polypeptide comprising 8, 9 or all 10 mutations of the mutation set of VAR-30 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   416. A capsid polypeptide comprising 4, 5 or all 6 mutations of the mutation set of VAR-31 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   417. A capsid polypeptide comprising 4, 5 or all 6 mutations of the mutation set of VAR-32 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   418. A capsid polypeptide comprising 7, 8 or all 9 mutations of the mutation set of VAR-33 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   419. A capsid polypeptide comprising 5, 6 or all 7 mutations of the mutation set of VAR-34 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   420. A capsid polypeptide comprising 6, 7 or all 8 mutations of the mutation set of VAR-35 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   421. A capsid polypeptide comprising 5, 6 or all 7 mutations of the mutation set of VAR-36 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   422. A capsid polypeptide comprising 5, 6 or all 7 mutations of the mutation set of VAR-37 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   423. A capsid polypeptide comprising 5, 6 or all 7 mutations of the mutation set of VAR-38 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   424. A capsid polypeptide comprising 5, 6 or all 7 mutations of the mutation set of VAR-39 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   425. A capsid polypeptide comprising 4, 5 or all 6 mutations of the mutation set of VAR-40 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   426. A capsid polypeptide comprising 7, 8 or all 9 mutations of the mutation set of VAR-41 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   427. A capsid polypeptide comprising 4, 5 or all 6 mutations of the mutation set of VAR-42 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   428. A capsid polypeptide comprising 5, 6 or all 7 mutations of the mutation set of VAR-43 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   429. A capsid polypeptide comprising 5, 6 or all 7 mutations of the mutation set of VAR-44 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   430. A capsid polypeptide comprising 7, 8 or all 9 mutations of the mutation set of VAR-45 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   431. A capsid polypeptide comprising 5, 6 or all 7 mutations of the mutation set of VAR-46 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   432. A capsid polypeptide comprising 7, 8 or all 9 mutations of the mutation set of VAR-47 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   433. A capsid polypeptide comprising 6, 7 or all 8 mutations of the mutation set of VAR-48 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   434. A capsid polypeptide comprising 6, 7 or all 8 mutations of the mutation set of VAR-49 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   435. A capsid polypeptide comprising 5, 6 or all 7 mutations of the mutation set of VAR-50 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   436. A capsid polypeptide comprising 6, 7 or all 8 mutations of the mutation set of VAR-51 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   437. A capsid polypeptide comprising 6, 7 or all 8 mutations of the mutation set of VAR-52 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   438. A capsid polypeptide comprising 5, 6 or all 7 mutations of the mutation set of VAR-53 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   439. A capsid polypeptide comprising 6, 7 or all 8 mutations of the mutation set of VAR-54 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   440. A capsid polypeptide comprising 5, 6 or all 7 mutations of the mutation set of VAR-55 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   441. A capsid polypeptide comprising 5, 6 or all 7 mutations of the mutation set of VAR-56 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   442. A capsid polypeptide comprising 4, 5 or all 6 mutations of the mutation set of VAR-57 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   443. A capsid polypeptide comprising 3, 4 or all 5 mutations of the mutation set of VAR-58 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   444. A capsid polypeptide comprising 5, 6 or all 7 mutations of the mutation set of VAR-59 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   445. A capsid polypeptide comprising 6, 7 or all 8 mutations of the mutation set of VAR-60 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   446. A capsid polypeptide comprising 6, 7 or all 8 mutations of the mutation set of VAR-61 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   447. A capsid polypeptide comprising 4, 5 or all 6 mutations of the mutation set of VAR-62 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   448. In a capsid polypeptide, the improvement comprising all mutations, all mutations except one mutation, or all mutations except two mutations of the mutation set of any one of VAR-1 to VAR-62 as compared to the VP1 capsid polypeptide of SEQ ID NO:1, as set forth in Table 27, preferably wherein the mutations comprise the V596 and N598 mutations and optionally a mutation at Q579 and/or T593 as compared to the VP1 capsid polypeptide of SEQ ID NO:1.
   449. The capsid polypeptide of any one of embodiments 324 to 448, comprising an amino acid sequence having at least 70% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   450. The capsid polypeptide of any one of embodiments 324 to 448, comprising an amino acid sequence having at least 75% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   451. The capsid polypeptide of any one of embodiments 324 to 448, comprising an amino acid sequence having at least 80% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   452. The capsid polypeptide of any one of embodiments 324 to 448, comprising an amino acid sequence having at least 85% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   453. The capsid polypeptide of any one of embodiments 324 to 448, comprising an amino acid sequence having at least 90% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   454. The capsid polypeptide of any one of embodiments 324 to 448, comprising an amino acid sequence having at least 91% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   455. The capsid polypeptide of any one of embodiments 324 to 448, comprising an amino acid sequence having at least 92% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   456. The capsid polypeptide of any one of embodiments 324 to 448, comprising an amino acid sequence having at least 93% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   457. The capsid polypeptide of any one of embodiments 324 to 448, comprising an amino acid sequence having at least 94% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   458. The capsid polypeptide of any one of embodiments 324 to 448, comprising an amino acid sequence having at least 95% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   459. The capsid polypeptide of any one of embodiments 324 to 448, comprising an amino acid sequence having at least 96% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   460. The capsid polypeptide of any one of embodiments 324 to 448, comprising an amino acid sequence having at least 97% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   461. The capsid polypeptide of any one of embodiments 324 to 448, comprising an amino acid sequence having at least 98% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   462. The capsid polypeptide of any one of embodiments 324 to 448, comprising an amino acid sequence having at least 99% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
   463. The capsid polypeptide of any one of embodiments 1 to 462, whose sequence has an edit distance of (a) 12 or lower or (b) 10 or lower to a VP1 capsid polypeptide of SEQ ID NO:1 or the VP2 or VP3 portion thereof.
   464. The capsid polypeptide of any one of embodiments 1 to 463, whose sequence has an edit distance of (a) 12 or lower or (b) 10 or lower to:
(a) a VP1 capsid polypeptide of SEQ ID NO:12;
(b) a VP2 capsid polypeptide corresponding to the VP2 portion of SEQ ID NO:12; or
(c) a VP3 capsid polypeptide corresponding to the VP3 portion of SEQ ID NO:12.
   465. The capsid polypeptide of any one of embodiments 1 to 463, whose sequence has an edit distance of (a) 12 or lower or (b) 10 or lower to:
(a) a VP1 capsid polypeptide of SEQ ID NO:13;
(b) a VP2 capsid polypeptide corresponding to the VP2 portion of SEQ ID NO:13; or
(c) a VP3 capsid polypeptide corresponding to the VP3 portion of SEQ ID NO:13.
   466. The capsid polypeptide of any one of embodiments 1 to 463, whose sequence has an edit distance of (a) 12 or lower or (b) 10 or lower to:
(a) a VP1 capsid polypeptide of SEQ ID NO:14;
(b) a VP2 capsid polypeptide corresponding to the VP2 portion of SEQ ID NO:14; or
(c) a VP3 capsid polypeptide corresponding to the VP3 portion of SEQ ID NO:14.
   467. The capsid polypeptide of any one of embodiments 1 to 463, whose sequence has an edit distance of (a) 12 or lower or (b) 10 or lower to:
(a) a VP1 capsid polypeptide of SEQ ID NO:15;
(b) a VP2 capsid polypeptide corresponding to the VP2 portion of SEQ ID NO:15; or
(c) a VP3 capsid polypeptide corresponding to the VP3 portion of SEQ ID NO:15.
   468. The capsid polypeptide of any one of embodiments 1 to 463, whose sequence has an edit distance of (a) 12 or lower or (b) 10 or lower to:
(a) a VP1 capsid polypeptide of SEQ ID NO:16;
(b) a VP2 capsid polypeptide corresponding to the VP2 portion of SEQ ID NO:16; or
(c) a VP3 capsid polypeptide corresponding to the VP3 portion of SEQ ID NO:16.
   469. The capsid polypeptide of any one of embodiments 1 to 463, whose sequence has an edit distance of (a) 12 or lower or (b) 10 or lower to:
(a) a VP1 capsid polypeptide of SEQ ID NO:17;
(b) a VP2 capsid polypeptide corresponding to the VP2 portion of SEQ ID NO:17; or
(c) a VP3 capsid polypeptide corresponding to the VP3 portion of SEQ ID NO:17.
   470. The capsid polypeptide of any one of embodiments 1 to 463, whose sequence has an edit distance of (a) 12 or lower or (b) 10 or lower to:
(a) a VP1 capsid polypeptide of SEQ ID NO:18;
(b) a VP2 capsid polypeptide corresponding to the VP2 portion of SEQ ID NO:18; or
(c) a VP3 capsid polypeptide corresponding to the VP3 portion of SEQ ID NO:18.
   471. The capsid polypeptide of any one of embodiments 1 to 463, whose sequence has an edit distance of (a) 12 or lower or (b) 10 or lower to:
(a) a VP1 capsid polypeptide of SEQ ID NO:19;
(b) a VP2 capsid polypeptide corresponding to the VP2 portion of SEQ ID NO:19; or
(c) a VP3 capsid polypeptide corresponding to the VP3 portion of SEQ ID NO:19.
   472. The capsid polypeptide of any one of embodiments 1 to 463, whose sequence has an edit distance of (a) 12 or lower or (b) 10 or lower to:
(a) a VP1 capsid polypeptide of SEQ ID NO:20;
(b) a VP2 capsid polypeptide corresponding to the VP2 portion of SEQ ID NO:20; or
(c) a VP3 capsid polypeptide corresponding to the VP3 portion of SEQ ID NO:20.
   473. The capsid polypeptide of any one of embodiments 1 to 463, whose sequence has an edit distance of (a) 12 or lower or (b) 10 or lower to:
(a) a VP1 capsid polypeptide of SEQ ID NO:21;
(b) a VP2 capsid polypeptide corresponding to the VP2 portion of SEQ ID NO:21; or
(c) a VP3 capsid polypeptide corresponding to the VP3 portion of SEQ ID NO:21
   474. The capsid polypeptide of any one of embodiments 1 to 463, whose sequence has an edit distance of (a) 12 or lower or (b) 10 or lower to:
(a) a VP1 capsid polypeptide of SEQ ID NO:22;
(b) a VP2 capsid polypeptide corresponding to the VP2 portion of SEQ ID NO:22; or
(c) a VP3 capsid polypeptide corresponding to the VP3 portion of SEQ ID NO:22.
   475. The capsid polypeptide of any one of embodiments 1 to 463, whose sequence has an edit distance of (a) 12 or lower or (b) 10 or lower to:
(a) a VP1 capsid polypeptide of SEQ ID NO:23;
(b) a VP2 capsid polypeptide corresponding to the VP2 portion of SEQ ID NO:23; or
(c) a VP3 capsid polypeptide corresponding to the VP3 portion of SEQ ID NO:23.
   476. The capsid polypeptide of any one of embodiments 1 to 463, whose sequence has an edit distance of (a) 12 or lower or (b) 10 or lower to:
(a) a VP1 capsid polypeptide of SEQ ID NO:24;
(b) a VP2 capsid polypeptide corresponding to the VP2 portion of SEQ ID NO:24; or
(c) a VP3 capsid polypeptide corresponding to the VP3 portion of SEQ ID NO:24.
   477. The capsid polypeptide of any one of embodiments 1 to 463, whose sequence has an edit distance of (a) 12 or lower or (b) 10 or lower to:
(a) a VP1 capsid polypeptide of SEQ ID NO:25;
(b) a VP2 capsid polypeptide corresponding to the VP2 portion of SEQ ID NO:25, or
(c) a VP3 capsid polypeptide corresponding to the VP3 portion of SEQ ID NO:25.
   478. The capsid polypeptide of any one of embodiments 1 to 463, whose sequence has an edit distance of (a) 12 or lower or (b) 10 or lower to:
(a) a VP1 capsid polypeptide of SEQ ID NO:26;
(b) a VP2 capsid polypeptide corresponding to the VP2 portion of SEQ ID NO:26; or
(c) a VP3 capsid polypeptide corresponding to the VP3 portion of SEQ ID NO:26.
   479. The capsid polypeptide of any one of embodiments 1 to 463, whose sequence has an edit distance of (a) 12 or lower or (b) 10 or lower to:
(a) a VP1 capsid polypeptide of SEQ ID NO:27;
(b) a VP2 capsid polypeptide corresponding to the VP2 portion of SEQ ID NO:27; or
(c) a VP3 capsid polypeptide corresponding to the VP3 portion of SEQ ID NO:27.
   480. The capsid polypeptide of any one of embodiments 1 to 463, whose sequence has an edit distance of (a) 12 or lower or (b) 10 or lower to:
(a) a VP1 capsid polypeptide of SEQ ID NO:28,
(b) a VP2 capsid polypeptide corresponding to the VP2 portion of SEQ ID NO:28; or
(c) a VP3 capsid polypeptide corresponding to the VP3 portion of SEQ ID NO:28.
   481. The capsid polypeptide of any one of embodiments 1 to 463, whose sequence has an edit distance of (a) 12 or lower or (b) 10 or lower to:
(a) a VP1 capsid polypeptide of SEQ ID NO:29;
(b) a VP2 capsid polypeptide corresponding to the VP2 portion of SEQ ID NO:29; or
(c) a VP3 capsid polypeptide corresponding to the VP3 portion of SEQ ID NO:29.
   482. The capsid polypeptide of any one of embodiments 1 to 463, whose sequence has an edit distance of (a) 12 or lower or (b) 10 or lower to:
(a) a VP1 capsid polypeptide of SEQ ID NO:30;
(b) a VP2 capsid polypeptide corresponding to the VP2 portion of SEQ ID NO:30; or
(c) a VP3 capsid polypeptide corresponding to the VP3 portion of SEQ ID NO:30.
   483. The capsid polypeptide of any one of embodiments 1 to 463, whose sequence has an edit distance of (a) 12 or lower or (b) 10 or lower to:
(a) a VP1 capsid polypeptide of SEQ ID NO:31;
(b) a VP2 capsid polypeptide corresponding to the VP2 portion of SEQ ID NO:31; or
(c) a VP3 capsid polypeptide corresponding to the VP3 portion of SEQ ID NO:31.
   484. The capsid polypeptide of any one of embodiments 1 to 463, whose sequence has an edit distance of (a) 12 or lower or (b) 10 or lower to:
(a) a VP1 capsid polypeptide of SEQ ID NO:32;
(b) a VP2 capsid polypeptide corresponding to the VP2 portion of SEQ ID NO:32, or
(c) a VP3 capsid polypeptide corresponding to the VP3 portion of SEQ ID NO:32.
   485. The capsid polypeptide of any one of embodiments 1 to 463, whose sequence has an edit distance of (a) 12 or lower or (b) 10 or lower to:
(a) a VP1 capsid polypeptide of SEQ ID NO:33;
(b) a VP2 capsid polypeptide corresponding to the VP2 portion of SEQ ID NO:33; or
(c) a VP3 capsid polypeptide corresponding to the VP3 portion of SEQ ID NO:33.
   486. The capsid polypeptide of any one of embodiments 1 to 463, whose sequence has an edit distance of (a) 12 or lower or (b) 10 or lower to:
(a) a VP1 capsid polypeptide of SEQ ID NO:34;
(b) a VP2 capsid polypeptide corresponding to the VP2 portion of SEQ ID NO:34; or
(c) a VP3 capsid polypeptide corresponding to the VP3 portion of SEQ ID NO:34.
   487. The capsid polypeptide of any one of embodiments 1 to 463, whose sequence has an edit distance of (a) 12 or lower or (b) 10 or lower to:
(a) a VP1 capsid polypeptide of SEQ ID NO:35,
(b) a VP2 capsid polypeptide corresponding to the VP2 portion of SEQ ID NO:35; or
(c) a VP3 capsid polypeptide corresponding to the VP3 portion of SEQ ID NO:35.
   488. The capsid polypeptide of any one of embodiments 1 to 463, whose sequence has an edit distance of (a) 12 or lower or (b) 10 or lower to:
(a) a VP1 capsid polypeptide of SEQ ID NO:36;
(b) a VP2 capsid polypeptide corresponding to the VP2 portion of SEQ ID NO:36; or
(c) a VP3 capsid polypeptide corresponding to the VP3 portion of SEQ ID NO:36.
   489. The capsid polypeptide of any one of embodiments 1 to 463, whose sequence has an edit distance of (a) 12 or lower or (b) 10 or lower to:
(a) a VP1 capsid polypeptide of SEQ ID NO:37;
(b) a VP2 capsid polypeptide corresponding to the VP2 portion of SEQ ID NO:37; or
(c) a VP3 capsid polypeptide corresponding to the VP3 portion of SEQ ID NO:37.
   490. The capsid polypeptide of any one of embodiments 1 to 463, whose sequence has an edit distance of (a) 12 or lower or (b) 10 or lower to:
(a) a VP1 capsid polypeptide of SEQ ID NO:38;
(b) a VP2 capsid polypeptide corresponding to the VP2 portion of SEQ ID NO:38; or
(c) a VP3 capsid polypeptide corresponding to the VP3 portion of SEQ ID NO:38.
   491. The capsid polypeptide of any one of embodiments 1 to 463, whose sequence has an edit distance of (a) 12 or lower or (b) 10 or lower to:
(a) a VP1 capsid polypeptide of SEQ ID NO:39;
(b) a VP2 capsid polypeptide corresponding to the VP2 portion of SEQ ID NO:39, or
(c) a VP3 capsid polypeptide corresponding to the VP3 portion of SEQ ID NO:39.
   492. The capsid polypeptide of any one of embodiments 1 to 463, whose sequence has an edit distance of (a) 12 or lower or (b) 10 or lower to:
(a) a VP1 capsid polypeptide of SEQ ID NO:40;
(b) a VP2 capsid polypeptide corresponding to the VP2 portion of SEQ ID NO:40; or
(c) a VP3 capsid polypeptide corresponding to the VP3 portion of SEQ ID NO:40.
   493. The capsid polypeptide of any one of embodiments 1 to 463, whose sequence has an edit distance of (a) 12 or lower or (b) 10 or lower to:
(a) a VP1 capsid polypeptide of SEQ ID NO:41;
(b) a VP2 capsid polypeptide corresponding to the VP2 portion of SEQ ID NO:41; or
(c) a VP3 capsid polypeptide corresponding to the VP3 portion of SEQ ID NO:41.
   494. The capsid polypeptide of any one of embodiments 1 to 463, whose sequence has an edit distance of (a) 12 or lower or (b) 10 or lower to:
(a) a VP1 capsid polypeptide of SEQ ID NO:42,
(b) a VP2 capsid polypeptide corresponding to the VP2 portion of SEQ ID NO:42; or
(c) a VP3 capsid polypeptide corresponding to the VP3 portion of SEQ ID NO:42.
   495. The capsid polypeptide of any one of embodiments 1 to 463, whose sequence has an edit distance of (a) 12 or lower or (b) 10 or lower to:
(a) a VP1 capsid polypeptide of SEQ ID NO:43;
(b) a VP2 capsid polypeptide corresponding to the VP2 portion of SEQ ID NO:43; or
(c) a VP3 capsid polypeptide corresponding to the VP3 portion of SEQ ID NO:43.
   496. The capsid polypeptide of any one of embodiments 1 to 463, whose sequence has an edit distance of (a) 12 or lower or (b) 10 or lower to:
(a) a VP1 capsid polypeptide of SEQ ID NO:44;
(b) a VP2 capsid polypeptide corresponding to the VP2 portion of SEQ ID NO:44; or
(c) a VP3 capsid polypeptide corresponding to the VP3 portion of SEQ ID NO:44.
   497. The capsid polypeptide of any one of embodiments 1 to 463, whose sequence has an edit distance of (a) 12 or lower or (b) 10 or lower to:
(a) a VP1 capsid polypeptide of SEQ ID NO:45;
(b) a VP2 capsid polypeptide corresponding to the VP2 portion of SEQ ID NO:45; or
(c) a VP3 capsid polypeptide corresponding to the VP3 portion of SEQ ID NO:45.
   498. The capsid polypeptide of any one of embodiments 1 to 463, whose sequence has an edit distance of (a) 12 or lower or (b) 10 or lower to:
(a) a VP1 capsid polypeptide of SEQ ID NO:46;
(b) a VP2 capsid polypeptide corresponding to the VP2 portion of SEQ ID NO:46, or
(c) a VP3 capsid polypeptide corresponding to the VP3 portion of SEQ ID NO:46.
   499. The capsid polypeptide of any one of embodiments 1 to 463, whose sequence has an edit distance of (a) 12 or lower or (b) 10 or lower to:
(a) a VP1 capsid polypeptide of SEQ ID NO:47;
(b) a VP2 capsid polypeptide corresponding to the VP2 portion of SEQ ID NO:47; or
(c) a VP3 capsid polypeptide corresponding to the VP3 portion of SEQ ID NO:47.
   500. The capsid polypeptide of any one of embodiments 1 to 463, whose sequence has an edit distance of (a) 12 or lower or (b) 10 or lower to:
(a) a VP1 capsid polypeptide of SEQ ID NO:48;
(b) a VP2 capsid polypeptide corresponding to the VP2 portion of SEQ ID NO:48; or
(c) a VP3 capsid polypeptide corresponding to the VP3 portion of SEQ ID NO:48.
   501. The capsid polypeptide of any one of embodiments 1 to 463, whose sequence has an edit distance of (a) 12 or lower or (b) 10 or lower to:
(a) a VP1 capsid polypeptide of SEQ ID NO:49;
(b) a VP2 capsid polypeptide corresponding to the VP2 portion of SEQ ID NO:49; or
(c) a VP3 capsid polypeptide corresponding to the VP3 portion of SEQ ID NO:49.
   502. The capsid polypeptide of any one of embodiments 1 to 463, whose sequence has an edit distance of (a) 12 or lower or (b) 10 or lower to:
(a) a VP1 capsid polypeptide of SEQ ID NO:50;
(b) a VP2 capsid polypeptide corresponding to the VP2 portion of SEQ ID NO:50, or
(c) a VP3 capsid polypeptide corresponding to the VP3 portion of SEQ ID NO:50.
   503. The capsid polypeptide of any one of embodiments 1 to 463, whose sequence has an edit distance of (a) 12 or lower or (b) 10 or lower to:
(a) a VP1 capsid polypeptide of SEQ ID NO:51;
(b) a VP2 capsid polypeptide corresponding to the VP2 portion of SEQ ID NO:51; or
(c) a VP3 capsid polypeptide corresponding to the VP3 portion of SEQ ID NO:51.
   504. The capsid polypeptide of any one of embodiments 1 to 463, whose sequence has an edit distance of (a) 12 or lower or (b) 10 or lower to:
(a) a VP1 capsid polypeptide of SEQ ID NO:52;
(b) a VP2 capsid polypeptide corresponding to the VP2 portion of SEQ ID NO:52; or
(c) a VP3 capsid polypeptide corresponding to the VP3 portion of SEQ ID NO:52.
   505. The capsid polypeptide of any one of embodiments 1 to 463, whose sequence has an edit distance of (a) 12 or lower or (b) 10 or lower to:
(a) a VP1 capsid polypeptide of SEQ ID NO:53,
(b) a VP2 capsid polypeptide corresponding to the VP2 portion of SEQ ID NO:53; or
(c) a VP3 capsid polypeptide corresponding to the VP3 portion of SEQ ID NO:53.
   506. The capsid polypeptide of any one of embodiments 1 to 463, whose sequence has an edit distance of (a) 12 or lower or (b) 10 or lower to:
(a) a VP1 capsid polypeptide of SEQ ID NO:54;
(b) a VP2 capsid polypeptide corresponding to the VP2 portion of SEQ ID NO:54; or
(c) a VP3 capsid polypeptide corresponding to the VP3 portion of SEQ ID NO:54.
   507. The capsid polypeptide of any one of embodiments 1 to 463, whose sequence has an edit distance of (a) 12 or lower or (b) 10 or lower to:
(a) a VP1 capsid polypeptide of SEQ ID NO:55;
(b) a VP2 capsid polypeptide corresponding to the VP2 portion of SEQ ID NO:55; or
(c) a VP3 capsid polypeptide corresponding to the VP3 portion of SEQ ID NO:55.
   508. The capsid polypeptide of any one of embodiments 1 to 463, whose sequence has an edit distance of (a) 12 or lower or (b) 10 or lower to:
(a) a VP1 capsid polypeptide of SEQ ID NO:56;
(b) a VP2 capsid polypeptide corresponding to the VP2 portion of SEQ ID NO:56; or
(c) a VP3 capsid polypeptide corresponding to the VP3 portion of SEQ ID NO:56.
   509. The capsid polypeptide of any one of embodiments 1 to 463, whose sequence has an edit distance of (a) 12 or lower or (b) 10 or lower to:
(a) a VP1 capsid polypeptide of SEQ ID NO:57;
(b) a VP2 capsid polypeptide corresponding to the VP2 portion of SEQ ID NO:57, or
(c) a VP3 capsid polypeptide corresponding to the VP3 portion of SEQ ID NO:57.
   510. The capsid polypeptide of any one of embodiments 1 to 463, whose sequence has an edit distance of (a) 12 or lower or (b) 10 or lower to:
(a) a VP1 capsid polypeptide of SEQ ID NO:58;
(b) a VP2 capsid polypeptide corresponding to the VP2 portion of SEQ ID NO:58; or
(c) a VP3 capsid polypeptide corresponding to the VP3 portion of SEQ ID NO:58.
   511. The capsid polypeptide of any one of embodiments 1 to 463, whose sequence has an edit distance of (a) 12 or lower or (b) 10 or lower to:
(a) a VP1 capsid polypeptide of SEQ ID NO:59;
(b) a VP2 capsid polypeptide corresponding to the VP2 portion of SEQ ID NO:59; or
(c) a VP3 capsid polypeptide corresponding to the VP3 portion of SEQ ID NO:59. (d)
   512. The capsid polypeptide of any one of embodiments 1 to 463, whose sequence has an edit distance of (a) 12 or lower or (b) 10 or lower to:
(a) a VP1 capsid polypeptide of SEQ ID NO:60;
(b) a VP2 capsid polypeptide corresponding to the VP2 portion of SEQ ID NO:60; or
(c) a VP3 capsid polypeptide corresponding to the VP3 portion of SEQ ID NO:60.
   513. The capsid polypeptide of any one of embodiments 1 to 463, whose sequence has an edit distance of (a) 12 or lower or (b) 10 or lower to:
(a) a VP1 capsid polypeptide of SEQ ID NO:61;
(b) a VP2 capsid polypeptide corresponding to the VP2 portion of SEQ ID NO:61; or
(c) a VP3 capsid polypeptide corresponding to the VP3 portion of SEQ ID NO:61.
   514. The capsid polypeptide of any one of embodiments 1 to 463, whose sequence has an edit distance of (a) 12 or lower or (b) 10 or lower to:
(a) a VP1 capsid polypeptide of SEQ ID NO:62;
(b) a VP2 capsid polypeptide corresponding to the VP2 portion of SEQ ID NO:62; or
(c) a VP3 capsid polypeptide corresponding to the VP3 portion of SEQ ID NO:62.
   515. The capsid polypeptide of any one of embodiments 1 to 463, whose sequence has an edit distance of (a) 12 or lower or (b) 10 or lower to:
(a) a VP1 capsid polypeptide of SEQ ID NO:63;
(b) a VP2 capsid polypeptide corresponding to the VP2 portion of SEQ ID NO:63; or
(c) a VP3 capsid polypeptide corresponding to the VP3 portion of SEQ ID NO:63.
   516. The capsid polypeptide of any one of embodiments 1 to 463, whose sequence has an edit distance of (a) 12 or lower or (b) 10 or lower to:
(a) a VP1 capsid polypeptide of SEQ ID NO:64,
(b) a VP2 capsid polypeptide corresponding to the VP2 portion of SEQ ID NO:64; or
(c) a VP3 capsid polypeptide corresponding to the VP3 portion of SEQ ID NO:64.
   517. The capsid polypeptide of any one of embodiments 1 to 463, whose sequence has an edit distance of (a) 12 or lower or (b) 10 or lower to:
(a) a VP1 capsid polypeptide of SEQ ID NO:65;
(b) a VP2 capsid polypeptide corresponding to the VP2 portion of SEQ ID NO:65; or
(c) a VP3 capsid polypeptide corresponding to the VP3 portion of SEQ ID NO:65.
   518. The capsid polypeptide of any one of embodiments 1 to 463, whose sequence has an edit distance of (a) 12 or lower or (b) 10 or lower to:
(a) a VP1 capsid polypeptide of SEQ ID NO:66;
(b) a VP2 capsid polypeptide corresponding to the VP2 portion of SEQ ID NO:66; or
(c) a VP3 capsid polypeptide corresponding to the VP3 portion of SEQ ID NO:66.
   519. The capsid polypeptide of any one of embodiments 1 to 463, whose sequence has an edit distance of (a) 12 or lower or (b) 10 or lower to:
(a) a VP1 capsid polypeptide of SEQ ID NO:67;
(b) a VP2 capsid polypeptide corresponding to the VP2 portion of SEQ ID NO:67; or
(c) a VP3 capsid polypeptide corresponding to the VP3 portion of SEQ ID NO:67.
   520. The capsid polypeptide of any one of embodiments 1 to 463, whose sequence has an edit distance of (a) 12 or lower or (b) 10 or lower to:
(a) a VP1 capsid polypeptide of SEQ ID NO:68;
(b) a VP2 capsid polypeptide corresponding to the VP2 portion of SEQ ID NO:68, or
(c) a VP3 capsid polypeptide corresponding to the VP3 portion of SEQ ID NO:68.
   521. The capsid polypeptide of any one of embodiments 1 to 463, whose sequence has an edit distance of (a) 12 or lower or (b) 10 or lower to:
(a) a VP1 capsid polypeptide of SEQ ID NO:69;
(b) a VP2 capsid polypeptide corresponding to the VP2 portion of SEQ ID NO:69; or
(c) a VP3 capsid polypeptide corresponding to the VP3 portion of SEQ ID NO:69.
   522. The capsid polypeptide of any one of embodiments 1 to 463, whose sequence has an edit distance of (a) 12 or lower or (b) 10 or lower to:
(a) a VP1 capsid polypeptide of SEQ ID NO:70;
(b) a VP2 capsid polypeptide corresponding to the VP2 portion of SEQ ID NO:70; or
(c) a VP3 capsid polypeptide corresponding to the VP3 portion of SEQ ID NO:70.
   523. The capsid polypeptide of any one of embodiments 1 to 463, whose sequence has an edit distance of (a) 12 or lower or (b) 10 or lower to:
(a) a VP1 capsid polypeptide of SEQ ID NO:71;
(b) a VP2 capsid polypeptide corresponding to the VP2 portion of SEQ ID NO:71; or
(c) a VP3 capsid polypeptide corresponding to the VP3 portion of SEQ ID NO:71.
   524. The capsid polypeptide of any one of embodiments 1 to 463, whose sequence has an edit distance of (a) 12 or lower or (b) 10 or lower to:
(a) a VP1 capsid polypeptide of SEQ ID NO:72;
(b) a VP2 capsid polypeptide corresponding to the VP2 portion of SEQ ID NO:72, or
(c) a VP3 capsid polypeptide corresponding to the VP3 portion of SEQ ID NO:72.
   525. The capsid polypeptide of any one of embodiments 1 to 463, whose sequence has an edit distance of (a) 12 or lower or (b) 10 or lower to:
(a) a VP1 capsid polypeptide of SEQ ID NO:73;
(b) a VP2 capsid polypeptide corresponding to the VP2 portion of SEQ ID NO:73; or
(c) a VP3 capsid polypeptide corresponding to the VP3 portion of SEQ ID NO:73.
   526. The capsid polypeptide of any one of embodiments 1 to 525, which does not consist of the amino acid sequence of any one of SEQ ID NOs:136 to 252, or the VP2 or VP3 portion thereof.
   527. The capsid polypeptide of any one of embodiments 1 to 526, which is a VP1 capsid polypeptide.
   528. The capsid polypeptide of any one of embodiments 1 to 526, which is a VP2 capsid polypeptide.
   529. The capsid polypeptide of any one of embodiments 1 to 526, which is a VP3 capsid polypeptide.
   530. The capsid polypeptide of any one of embodiments 1 to 529, which lacks a targeting peptide.
   531. The capsid polypeptide of any one of embodiments 1 to 529, which comprises a targeting peptide, optionally wherein (a) the targeting peptide comprises or consists of the amino acid sequence of any one of SEQ ID NOs:255-262; (b) the targeting peptide is inserted, immediately subsequent to the position corresponding to 586, 588, or 589 of SEQ ID NO:1; (c) the capsid polypeptide further comprises a deletion at the position corresponding to 587 and/or 588 of SEQ ID NO:1; or (d) any combination of two or all three of (a), (b) and (c).
   532. A nucleic acid molecule encoding a capsid polypeptide of any one of embodiments 1 to 531.
   533. The nucleic acid molecule of embodiment 532, wherein the nucleic acid molecule comprises a nucleotide sequence having at least 70% sequence identity to the nucleic sequence of any one of SEQ ID NOs:74-135 or a fragment thereof (e.g., a VP1-encoding, a VP2-encoding or a VP3-encoding fragment thereof).
   534. The nucleic acid molecule of embodiment 532, wherein the nucleic acid molecule comprises a nucleotide sequence having at least 75% sequence identity to the nucleic sequence of any one of SEQ ID NOs:74-135 or a fragment thereof (e.g., a VP1-encoding, a VP2-encoding or a VP3-encoding fragment thereof).
   535. The nucleic acid molecule of embodiment 532, wherein the nucleic acid molecule comprises a nucleotide sequence having at least 80% sequence identity to the nucleic sequence of any one of SEQ ID NOs:74-135 or a fragment thereof (e.g., a VP1-encoding, a VP2-encoding or a VP3-encoding fragment thereof).
   536. The nucleic acid molecule of embodiment 532, wherein the nucleic acid molecule comprises a nucleotide sequence having at least 85% sequence identity to the nucleic sequence of any one of SEQ ID NOs:74-135 or a fragment thereof (e.g., a VP1-encoding, a VP2-encoding or a VP3-encoding fragment thereof).
   537. The nucleic acid molecule of embodiment 532, wherein the nucleic acid molecule comprises a nucleotide sequence having at least 90% sequence identity to the nucleic sequence of any one of SEQ ID NOs:74-135 or a fragment thereof (e.g., a VP1-encoding, a VP2-encoding or a VP3-encoding fragment thereof).
   538. The nucleic acid molecule of embodiment 532, wherein the nucleic acid molecule comprises a nucleotide sequence having at least 91% sequence identity to the nucleic sequence of any one of SEQ ID NOs:74-135 or a fragment thereof (e.g., a VP1-encoding, a VP2-encoding or a VP3-encoding fragment thereof).
   539. The nucleic acid molecule of embodiment 532, wherein the nucleic acid molecule comprises a nucleotide sequence having at least 92% sequence identity to the nucleic sequence of any one of SEQ ID NOs:74-135 or a fragment thereof (e.g., a VP1-encoding, a VP2-encoding or a VP3-encoding fragment thereof).
   540. The nucleic acid molecule of embodiment 532, wherein the nucleic acid molecule comprises a nucleotide sequence having at least 93% sequence identity to the nucleic sequence of any one of SEQ ID NOs:74-135 or a fragment thereof (e.g., a VP1-encoding, a VP2-encoding or a VP3-encoding fragment thereof).
   541. The nucleic acid molecule of embodiment 532, wherein the nucleic acid molecule comprises a nucleotide sequence having at least 94% sequence identity to the nucleic sequence of any one of SEQ ID NOs:74-135 or a fragment thereof (e.g., a VP1-encoding, a VP2-encoding or a VP3-encoding fragment thereof).
   542. The nucleic acid molecule of embodiment 532, wherein the nucleic acid molecule comprises a nucleotide sequence having at least 95% sequence identity to the nucleic sequence of any one of SEQ ID NOs:74-135 or a fragment thereof (e.g., a VP1-encoding, a VP2-encoding or a VP3-encoding fragment thereof).
   543. The nucleic acid molecule of embodiment 532, wherein the nucleic acid molecule comprises a nucleotide sequence having at least 96% sequence identity to the nucleic sequence of any one of SEQ ID NOs:74-135 or a fragment thereof (e.g., a VP1-encoding, a VP2-encoding or a VP3-encoding fragment thereof).
   544. The nucleic acid molecule of embodiment 532, wherein the nucleic acid molecule comprises a nucleotide sequence having at least 97% sequence identity to the nucleic sequence of any one of SEQ ID NOs:74-135 or a fragment thereof (e.g., a VP1-encoding, a VP2-encoding or a VP3-encoding fragment thereof).
   545. The nucleic acid molecule of embodiment 532, wherein the nucleic acid molecule comprises a nucleotide sequence having at least 98% sequence identity to the nucleic sequence of any one of SEQ ID NOs:74-135 or a fragment thereof (e.g., a VP1-encoding, a VP2-encoding or a VP3-encoding fragment thereof).
   546. The nucleic acid molecule of embodiment 532, wherein the nucleic acid molecule comprises a nucleotide sequence having at least 99% sequence identity to the nucleic sequence of any one of SEQ ID NOs:74-135 or a fragment thereof (e.g., a VP1-encoding, a VP2-encoding or a VP3-encoding fragment thereof).
   547. The nucleic acid molecule of embodiment 532, wherein the nucleic acid molecule comprises the nucleotide sequence of any one of SEQ ID NOs:74-135 or a fragment thereof (e.g., a VP1-encoding, a VP2-encoding or a VP3-encoding fragment thereof).
   548. The nucleic acid molecule of any one of embodiments 532 to 547, wherein the nucleic acid molecule comprises the nucleotide sequence of any one of SEQ ID NOs:74-135.
   549. The nucleic acid molecule of any one of embodiments 532 to 547, wherein the nucleic acid molecule comprises a fragment of the nucleotide sequence of any one of SEQ ID NOs:74-135 that encodes a VP2 capsid polypeptide.
   550. The nucleic acid molecule of any one of embodiments 532 to 547, wherein the nucleic acid molecule comprises a fragment of the nucleotide sequence of any one of SEQ ID NOs:74-135 that encodes a VP3 capsid polypeptide.
   551. The nucleic acid molecule of any one of embodiments 532 to 550, wherein the nucleic acid molecule is double-stranded or single-stranded, and wherein the nucleic acid molecule is linear or circular, e.g., wherein the nucleic acid molecule is a plasmid.
   552. A virus particle (e.g., adeno-associated virus (“AAV”) particle) comprising a capsid polypeptide of any one of embodiments 1 to 531, or comprising a capsid polypeptide encoded by the nucleic acid molecule of any one of embodiments 532 to 551.
   553. A virus particle (e.g., adeno-associated virus (“AAV”) particle) comprising (a) a variant capsid polypeptide having at least 70%, at least 75%, at least 80%, at least 85%, at least 90%, at least 91%, at least 92%, at least 93%, at least 94%, at least 95%, at least 96%, at least 97%, at least 98%, or at least 99% sequence identity ((i) calculated taking targeting peptide insertions into account or (ii) calculated without taking targeting peptide insertions into account) to a VP1 capsid polypeptide of SEQ ID NO:1 or the VP2 or VP3 portion thereof, (b) an encapsulated nucleic acid, and (c) means for causing the virus particle to have increased CNS biodistribution and/or CNS transduction and/or skeletal muscle biodistribution and/or skeletal muscle transduction, e.g., as measured in a mammal, e.g., in NHP, e.g., as described herein, relative to wild-type AAV9 (e.g., a virus particle comprising capsid polypeptides of SEQ ID NO:1 or encoded by SEQ ID NO:2), optionally wherein the increased CNS biodistribution and/or CNS transduction and/or skeletal muscle biodistribution and/or skeletal muscle transduction is measured using a method described in Section 5.4.
   554. The virus particle of embodiment 553, wherein (c) is means for causing the virus particle to have increased CNS (e.g., brain) biodistribution relative to wild-type AAV9 (e.g., a virus particle comprising capsid polypeptides of SEQ ID NO:1 or encoded by SEQ ID NO:2).
   555. The virus particle of embodiment 553, wherein (c) is means for causing the virus particle to have increased CNS (e.g., brain) transduction relative to wild-type AAV9 (e.g., a virus particle comprising capsid polypeptides of SEQ ID NO:1 or encoded by SEQ ID NO:2).
   556. The virus particle of embodiment 553, wherein (c) is means for causing the virus particle to have increased skeletal muscle biodistribution relative to wild-type AAV9 (e.g., a virus particle comprising capsid polypeptides of SEQ ID NO:1 or encoded by SEQ ID NO:2).
   557. The virus particle of embodiment 553, wherein (c) is means for causing the virus particle to have increased skeletal muscle transduction relative to wild-type AAV9 (e.g., a virus particle comprising capsid polypeptides of SEQ ID NO:1 or encoded by SEQ ID NO:2).
   558. The virus particle of any one of embodiments 553 to 557, wherein the encapsulated nucleic acid comprises a payload (e.g., a heterologous transgene) and one or more regulatory elements.
   559. The virus particle of embodiment 552, comprising a nucleic acid comprising a payload (e.g., a heterologous transgene) and one or more regulatory elements.
   560. The virus particle of embodiment 558 or embodiment 559, wherein the payload comprises a heterologous nucleic acid sequence as defined in any one of embodiments 1323 to 1805.
   561. The virus particle of any one of embodiments 558 to 560, wherein the one or more regulatory elements comprise a promoter.
   562. The virus particle of embodiment 561, wherein the promoter is a constitutive promoter.
   563. The virus particle of embodiment 562, wherein the promoter is a CBh promoter.
   564. The virus particle of embodiment 563, wherein the CBh promoter comprises a nucleotide sequence having at least 90%, at least 95%, at least 96%, at least 97%, or at least 98%, at least 99%, or 100% sequence identity to SEQ ID NO:253.
   565. The virus particle of embodiment 561, wherein the promoter is a CNS-specific promoter.
   566. The virus particle of embodiment 565, wherein the promoter is a hSYN promoter.
   567. The virus particle of embodiment 566, wherein the hSYN promoter comprises a nucleotide sequence having at least 90%, at least 95%, at least 96%, at least 97%, or at least 98%, at least 99%, or 100% sequence identity to SEQ ID NO:254.
   568. The virus particle of embodiment 561, wherein the promoter is a muscle-specific promoter.
   569. The virus particle of embodiment 568, wherein the muscle-specific promoter is a skeletal muscle-specific promoter.
   570. The virus particle of any one of embodiments 552 to 569, wherein said virus particle exhibits one or more features described in Section 5.4, for example wherein the one or more features comprise relative to wild-type AAV9 (e.g., a virus particle comprising capsid polypeptides of SEQ ID NO:1 or encoded by SEQ ID NO:2):
(a) an improvement in Category A (CNS biodistribution), e.g., as set forth in any one of embodiments A-1 to A-30;
(b) an improvement in Category B (CNS transduction), e.g., as set forth in any one of embodiments B-1 to B-32;
(c) an improvement in Category C (PNS biodistribution and/or transduction), e.g., as set forth in any one of embodiments C-1 to C-22;
(d) an improvement in Category D (liver biodistribution and/or transduction), e.g., as set forth in any one of embodiments D-1 to D-23;
(e) an improvement in Category E (spleen biodistribution and/or transduction); e.g., as set forth in any one of embodiments E-1 to E-22;
(f) an improvement in Category F (skeletal muscle biodistribution), e.g., as set forth in any one of embodiments F-1 to F-17;
(g) an improvement in Category G (skeletal muscle transduction), e.g., as set forth in any one of embodiments G-1 to G-9; or
(h) an improvement in any combination of two or more of:
   (i) an improvement in Category A (CNS biodistribution), e.g., as set forth in any one of embodiments A-1 to A-30;
   (ii) an improvement in Category B (CNS transduction), e.g., as set forth in any one of embodiments B-1 to B-32;
   (iii) an improvement in Category C (PNS biodistribution and/or transduction), e.g., as set forth in any one of embodiments C-1 to C-22;
   (iv) an improvement in Category D (liver biodistribution and/or transduction), e.g., as set forth in any one of embodiments D-1 to D-23;
   (v) an improvement in Category E (spleen biodistribution and/or transduction); e.g., as set forth in any one of embodiments E-1 to E-22;
   (vi) an improvement in Category F (skeletal muscle biodistribution), e.g., as set forth in any one of embodiments F-1 to F-17; and
   (vii) an improvement in Category G (skeletal muscle transduction), e.g., as set forth in any one of embodiments G-1 to G-9.
      571. The virus particle of embodiment 570, which exhibits increased central nervous system (CNS) biodistribution, e.g., as measured in a mammal, e.g., in mouse or in NHP, e.g., as described herein, relative to wild-type AAV9 (e.g., a virus particle comprising capsid polypeptides of SEQ ID NO:1 or encoded by SEQ ID NO:2), optionally wherein:
(a) the increase in CNS biodistribution is in an amount described in Section 5.4;
(b) the virus particle has features as defined in any one of embodiments A-1 to A-30;
(c) the increase in CNS biodistribution is measured using a method described in Section 5.4;
(d) the increased CNS biodistribution is exhibited upon systemic, e.g., intravenous, administration of said virus particle; or
(e) any combination of two or more, three or more or all four of (a) through (d).
   572. The virus particle of any one of embodiments 552 to 571, wherein said virus particle exhibits increased brain biodistribution, e.g., as measured in a mammal, e.g., in mouse or in NHP, e.g., as described herein, relative to wild-type AAV9 (e.g., a virus particle comprising capsid polypeptides of SEQ ID NO:1 or encoded by SEQ ID NO:2), optionally wherein the increased brain biodistribution (a) is in an amount described in Section 5.4, e.g., as set forth in any one of embodiments A-1 to A-30 and/or (b) is measured using a method described in Section 5.4.
   573. The virus particle of embodiment 572, wherein the increased brain biodistribution is exhibited upon systemic, e.g., intravenous, administration of said virus particle.
   574. The virus particle of embodiment 572 or embodiment 573, wherein the virus particle exhibits brain biodistribution that is at least 5-fold the brain biodistribution of a virus particle comprising capsid polypeptides of SEQ ID NO:1.
   575. The virus particle of embodiment 572 or embodiment 573, wherein the virus particle exhibits brain biodistribution that is at least 10-fold the brain biodistribution of a virus particle comprising capsid polypeptides of SEQ ID NO:1.
   576. The virus particle of embodiment 572 or embodiment 573, wherein the virus particle exhibits brain biodistribution that is at least 20-fold the brain biodistribution of a virus particle comprising capsid polypeptides of SEQ ID NO:1.
   577. The virus particle of embodiment 572 or embodiment 573, wherein the virus particle exhibits brain biodistribution that is at least 30-fold the brain biodistribution of a virus particle comprising capsid polypeptides of SEQ ID NO:1.
   578. The virus particle of embodiment 572 or embodiment 573, wherein the virus particle exhibits brain biodistribution that is at least 40-fold the brain biodistribution of a virus particle comprising capsid polypeptides of SEQ ID NO:1.
   579. The virus particle of embodiment 572 or embodiment 573, wherein the virus particle exhibits brain biodistribution that is at least 50-fold the brain biodistribution of a virus particle comprising capsid polypeptides of SEQ ID NO:1.
   580. The virus particle of embodiment 572 or embodiment 573, wherein the virus particle exhibits brain biodistribution that is at least 60-fold the brain biodistribution of a virus particle comprising capsid polypeptides of SEQ ID NO:1.
   581. The virus particle of any one of embodiments 572 to 579, wherein virus particle exhibits brain biodistribution that is up to 60-fold the brain biodistribution of a virus particle comprising capsid polypeptides of SEQ ID NO:1.
   582. The virus particle of any one of embodiments 572 to 580, wherein virus particle exhibits brain biodistribution that is up to 70-fold the brain biodistribution of a virus particle comprising capsid polypeptides of SEQ ID NO:1.
   583. The virus particle of any one of embodiments 572 to 580, wherein virus particle exhibits brain biodistribution that is up to 80-fold the brain biodistribution of a virus particle comprising capsid polypeptides of SEQ ID NO:1.
   584. The virus particle of any one of embodiments 572 to 580, wherein virus particle exhibits brain biodistribution that is up to 100-fold the brain biodistribution of a virus particle comprising capsid polypeptides of SEQ ID NO:1.
   585. The virus particle of any one of embodiments 572 to 580, wherein virus particle exhibits brain biodistribution that is up to 120-fold the brain biodistribution of a virus particle comprising capsid polypeptides of SEQ ID NO:1.
   586. The virus particle of any one of embodiments 552 to 585, wherein said virus particle exhibits decreased peripheral nervous system (“PNS”), e.g., dorsal root ganglia (DRG), biodistribution, e.g., as measured in a mammal, e.g., in mouse or in NHP, e.g., as described herein, relative to wild-type AAV9 (e.g., a virus particle comprising capsid polypeptides of SEQ ID NO:1 or encoded by SEQ ID NO:2), optionally wherein the decreased PNS, e.g., DRG, biodistribution (a) is in an amount described in Section 5.4, e.g., as set forth in any one of embodiments C-1 to C-30 and/or (b) is measured using a method described in Section 5.4.
   587. The virus particle of embodiment 586, wherein the reduced DRG biodistribution is exhibited upon systemic, e.g., intravenous, administration of said virus particle.
   588. The virus particle of embodiment 586 or embodiment 587, wherein the virus particle exhibits DRG biodistribution that is less than 1-fold the DRG biodistribution of a virus particle comprising capsid polypeptides of SEQ ID NO:1.
   589. The virus particle of embodiment 586 or embodiment 587, wherein the virus particle exhibits DRG biodistribution that is less than 0.9-fold the DRG biodistribution of a virus particle comprising capsid polypeptides of SEQ ID NO:1.
   590. The virus particle of embodiment 586 or embodiment 587, wherein the virus particle exhibits DRG biodistribution that is less than 0.8-fold the DRG biodistribution of a virus particle comprising capsid polypeptides of SEQ ID NO:1.
   591. The virus particle of embodiment 586 or embodiment 587, wherein the virus particle exhibits DRG biodistribution that is less than 0.7-fold the DRG biodistribution of a virus particle comprising capsid polypeptides of SEQ ID NO:1.
   592. The virus particle of embodiment 586 or embodiment 587, wherein the virus particle exhibits DRG biodistribution that is less than 0.6-fold the DRG biodistribution of a virus particle comprising capsid polypeptides of SEQ ID NO:1.
   593. The virus particle of embodiment 586 or embodiment 587, wherein the virus particle exhibits DRG biodistribution that is less than 0.5-fold the DRG biodistribution of a virus particle comprising capsid polypeptides of SEQ ID NO:1.
   594. The virus particle of embodiment 586 or embodiment 587, wherein the virus particle exhibits DRG biodistribution that is less than 0.4-fold the DRG biodistribution of a virus particle comprising capsid polypeptides of SEQ ID NO:1.
   595. The virus particle of embodiment 586 or embodiment 587, wherein the virus particle exhibits DRG biodistribution that is less than 0.3-fold the DRG biodistribution of a virus particle comprising capsid polypeptides of SEQ ID NO:1.
   596. The virus particle of embodiment 586 or embodiment 587, wherein the virus particle exhibits DRG biodistribution that is less than 0.2-fold the DRG biodistribution of a virus particle comprising capsid polypeptides of SEQ ID NO:1.
   597. The virus particle of embodiment 586 or embodiment 587, wherein the virus particle exhibits DRG biodistribution that is less than 0.1-fold the DRG biodistribution of a virus particle comprising capsid polypeptides of SEQ ID NO:1.
   598. The virus particle of any one of embodiments 586 to 596, wherein the virus particle exhibits DRG biodistribution that is at least 0.1-fold the DRG biodistribution of a virus particle comprising capsid polypeptides of SEQ ID NO:1.
   599. The virus particle of any one of embodiments 586 to 596, wherein the virus particle exhibits DRG biodistribution that is at least 0.13-fold the DRG biodistribution of a virus particle comprising capsid polypeptides of SEQ ID NO:1.
   600. The virus particle of any one of embodiments 586 to 596, wherein the virus particle exhibits DRG biodistribution that is at least 0.15-fold the DRG biodistribution of a virus particle comprising capsid polypeptides of SEQ ID NO:1.
   601. The virus particle of any one of embodiments 586 to 596, wherein the virus particle exhibits DRG biodistribution that is at least 0.17-fold the DRG biodistribution of a virus particle comprising capsid polypeptides of SEQ ID NO:1.
   602. The virus particle of any one of embodiments 586 to 595, wherein the virus particle exhibits DRG biodistribution that is at least 0.2-fold the DRG biodistribution of a virus particle comprising capsid polypeptides of SEQ ID NO:1.
   603. The virus particle of any one of embodiments 586 to 594, wherein the virus particle exhibits DRG biodistribution that is or at least 0.3-fold the DRG biodistribution of a virus particle comprising capsid polypeptides of SEQ ID NO:1.
   604. The virus particle of any one of embodiments 552 to 603, wherein said virus particle exhibits decreased liver biodistribution, e.g., as measured in a mammal, e.g., in mouse or in NHP, e.g., as described herein, relative to wild-type AAV9 (e.g., a virus particle comprising capsid polypeptides of SEQ ID NO:1 or encoded by SEQ ID NO:2), optionally wherein the decreased liver biodistribution (a) is in an amount described in Section 5.4, e.g., as set forth in any one of embodiments D-1 to D-30 and/or (b) is measured using a method described in Section 5.4.
   605. The virus particle of embodiment 604, wherein the decreased liver biodistribution is exhibited upon systemic, e.g., intravenous, administration of said virus particle.
   606. The virus particle of embodiment 604 or embodiment 605, wherein the virus particle exhibits liver biodistribution that is less than 1-fold the liver biodistribution of a virus particle comprising capsid polypeptides of SEQ ID NO:1.
   607. The virus particle of embodiment 604 or embodiment 605, wherein the virus particle exhibits liver biodistribution that is less than 0.9-fold the liver biodistribution of a virus particle comprising capsid polypeptides of SEQ ID NO:1.
   608. The virus particle of embodiment 604 or embodiment 605, wherein the virus particle exhibits liver biodistribution that is less than 0.8-fold the liver biodistribution of a virus particle comprising capsid polypeptides of SEQ ID NO:1.
   609. The virus particle of embodiment 604 or embodiment 605, wherein the virus particle exhibits liver biodistribution that is less than 0.7-fold the liver biodistribution of a virus particle comprising capsid polypeptides of SEQ ID NO:1.
   610. The virus particle of embodiment 604 or embodiment 605, wherein the virus particle exhibits liver biodistribution that is less than 0.6-fold the liver biodistribution of a virus particle comprising capsid polypeptides of SEQ ID NO:1.
   611. The virus particle of embodiment 604 or embodiment 605, wherein the virus particle exhibits liver biodistribution that is less than 0.5-fold the liver biodistribution of a virus particle comprising capsid polypeptides of SEQ ID NO:1.
   612. The virus particle of embodiment 604 or embodiment 605, wherein the virus particle exhibits liver biodistribution that is less than 0.4-fold the liver biodistribution of a virus particle comprising capsid polypeptides of SEQ ID NO:1.
   613. The virus particle of embodiment 604 or embodiment 605, wherein the virus particle exhibits liver biodistribution that is less than 0.3-fold the liver biodistribution of a virus particle comprising capsid polypeptides of SEQ ID NO:1.
   614. The virus particle of embodiment 604 or embodiment 605, wherein the virus particle exhibits liver biodistribution that is less than 0.2-fold the liver biodistribution of a virus particle comprising capsid polypeptides of SEQ ID NO:1.
   615. The virus particle of embodiment 604 or embodiment 605, wherein the virus particle exhibits liver biodistribution that is less than 0.1-fold the liver biodistribution of a virus particle comprising capsid polypeptides of SEQ ID NO:1.
   616. The virus particle of any one of embodiments 604 to 615, wherein the virus particle exhibits liver biodistribution that is at least 0.05-fold the liver biodistribution of a virus particle comprising capsid polypeptides of SEQ ID NO:1.
   617. The virus particle of any one of embodiments 604 to 615, wherein the virus particle exhibits liver biodistribution that is at least 0.06-fold the liver biodistribution of a virus particle comprising capsid polypeptides of SEQ ID NO:1.
   618. The virus particle of any one of embodiments 604 to 615, wherein the virus particle exhibits liver biodistribution that is at least 0.07-fold the liver biodistribution of a virus particle comprising capsid polypeptides of SEQ ID NO:1.
   619. The virus particle of any one of embodiments 604 to 615, wherein the virus particle exhibits liver biodistribution that is at least 0.08-fold the liver biodistribution of a virus particle comprising capsid polypeptides of SEQ ID NO:1.
   620. The virus particle of any one of embodiments 604 to 615, wherein the virus particle exhibits liver biodistribution that is at least 0.09-fold the liver biodistribution of a virus particle comprising capsid polypeptides of SEQ ID NO:1.
   621. The virus particle of any one of embodiments 604 to 614, wherein the virus particle exhibits liver biodistribution that is at least 0.1-fold the liver biodistribution of a virus particle comprising capsid polypeptides of SEQ ID NO:1.
   622. The virus particle of any one of embodiments 604 to 613, wherein the virus particle exhibits liver biodistribution that is at least 0.2-fold the liver biodistribution of a virus particle comprising capsid polypeptides of SEQ ID NO:1.
   623. The virus particle of any one of embodiments 604 to 612, wherein the virus particle exhibits liver biodistribution that is at least 0.3-fold the liver biodistribution of a virus particle comprising capsid polypeptides of SEQ ID NO:1.
   624. The virus particle of any one of embodiments 552 to 623, wherein said virus particle exhibits decreased spleen biodistribution, e.g., as measured in a mammal, e.g., in mouse or in NHP, e.g., as described herein, relative to wild-type AAV9 (e.g., a virus particle comprising capsid polypeptides of SEQ ID NO:1 or encoded by SEQ ID NO:2), optionally wherein the decreased spleen biodistribution (a) is in an amount described in Section 5.4, e.g., as set forth in any one of embodiments E-1 to E-22 and/or (b) is measured using a method described in Section 5.4.
   625. The virus particle of embodiment 624, wherein the decreased spleen biodistribution is exhibited upon systemic, e.g., intravenous, administration of said virus particle.
   626. The virus particle of embodiment 624 or embodiment 625, wherein the virus particle exhibits spleen biodistribution that is less than 1-fold the spleen biodistribution of a virus particle comprising capsid polypeptides of SEQ ID NO:1.
   627. The virus particle of embodiment 624 or embodiment 625, wherein the virus particle exhibits spleen biodistribution that is less than 0.9-fold the spleen biodistribution of a virus particle comprising capsid polypeptides of SEQ ID NO:1.
   628. The virus particle of embodiment 624 or embodiment 625, wherein the virus particle exhibits spleen biodistribution that is less than 0.8-fold the spleen biodistribution of a virus particle comprising capsid polypeptides of SEQ ID NO:1.
   629. The virus particle of embodiment 624 or embodiment 625, wherein the virus particle exhibits spleen biodistribution that is less than 0.7-fold the spleen biodistribution of a virus particle comprising capsid polypeptides of SEQ ID NO:1.
   630. The virus particle of embodiment 624 or embodiment 625, wherein the virus particle exhibits spleen biodistribution that is less than 0.6-fold the spleen biodistribution of a virus particle comprising capsid polypeptides of SEQ ID NO:1.
   631. The virus particle of embodiment 624 or embodiment 625, wherein the virus particle exhibits spleen biodistribution that is less than 0.5-fold the spleen biodistribution of a virus particle comprising capsid polypeptides of SEQ ID NO:1.
   632. The virus particle of embodiment 624 or embodiment 625, wherein the virus particle exhibits spleen biodistribution that is less than 0.4-fold the spleen biodistribution of a virus particle comprising capsid polypeptides of SEQ ID NO:1.
   633. The virus particle of embodiment 624 or embodiment 625, wherein the virus particle exhibits spleen biodistribution that is less than 0.3-fold the spleen biodistribution of a virus particle comprising capsid polypeptides of SEQ ID NO:1.
   634. The virus particle of embodiment 624 or embodiment 625, wherein the virus particle exhibits spleen biodistribution that is less than 0.2-fold the spleen biodistribution of a virus particle comprising capsid polypeptides of SEQ ID NO:1.
   635. The virus particle of embodiment 624 or embodiment 625, wherein the virus particle exhibits spleen biodistribution that is less than 0.1-fold the spleen biodistribution of a virus particle comprising capsid polypeptides of SEQ ID NO:1.
   636. The virus particle of any one of embodiments 624 to 635, wherein the virus particle exhibits spleen biodistribution that is at least 0.01-fold the spleen biodistribution of a virus particle comprising capsid polypeptides of SEQ ID NO:1.
   637. The virus particle of any one of embodiments 624 to 635, wherein the virus particle exhibits spleen biodistribution that is at least 0.015-fold the spleen biodistribution of a virus particle comprising capsid polypeptides of SEQ ID NO:1.
   638. The virus particle of any one of embodiments 624 to 635, wherein the virus particle exhibits spleen biodistribution that is at least 0.02-fold the spleen biodistribution of a virus particle comprising capsid polypeptides of SEQ ID NO:1.
   639. The virus particle of any one of embodiments 624 to 635, wherein the virus particle exhibits spleen biodistribution that is at least 0.05-fold the spleen biodistribution of a virus particle comprising capsid polypeptides of SEQ ID NO:1.
   640. The virus particle of any one of embodiments 624 to 635, wherein the virus particle exhibits spleen biodistribution that is at least 0.07-fold the spleen biodistribution of a virus particle comprising capsid polypeptides of SEQ ID NO:1.
   641. The virus particle of any one of embodiments 624 to 635, wherein the virus particle exhibits spleen biodistribution that is at least 0.09-fold the spleen biodistribution of a virus particle comprising capsid polypeptides of SEQ ID NO:1.
   642. The virus particle of any one of embodiments 624 to 634, wherein the virus particle exhibits spleen biodistribution that is at least 0.1-fold the spleen biodistribution of a virus particle comprising capsid polypeptides of SEQ ID NO:1.
   643. The virus particle of any one of embodiments 624 to 633, wherein the virus particle exhibits spleen biodistribution that is at least 0.2-fold the spleen biodistribution of a virus particle comprising capsid polypeptides of SEQ ID NO:1.
   644. The virus particle of any one of embodiments 624 to 632, wherein the virus particle exhibits spleen biodistribution that is at least 0.3-fold the spleen biodistribution of a virus particle comprising capsid polypeptides of SEQ ID NO:1.
   645. The virus particle of any one of embodiments 552 to 644, wherein said virus particle exhibits increased transduction in CNS e.g., as measured in a mammal, e.g., in mouse or in NHP, e.g., as described herein, relative to wild-type AAV9 (e.g., a virus particle comprising capsid polypeptides of SEQ ID NO:1 or encoded by SEQ ID NO:2), optionally wherein the increased CNS transduction (a) is in an amount described in Section 5.4, e.g., as set forth in any one of embodiments B-1 to B-32 and/or (b) is measured using a method described in Section 5.4.
   646. The virus particle of any one of embodiments 552 to 645, wherein said virus particle exhibits increased brain transduction, e.g., as measured in a mammal, e.g., in mouse or in NHP, e.g., as described herein, relative to wild-type AAV9 (e.g., a virus particle comprising capsid polypeptides of SEQ ID NO:1 or encoded by SEQ ID NO:2), optionally wherein the increased brain transduction (a) is in an amount described in Section 5.4, e.g., as set forth in any one of embodiments B-1 to B-32 and/or (b) is measured using a method described in Section 5.4.
   647. The virus particle of embodiment 646, wherein the increased brain transduction is exhibited upon systemic, e.g., intravenous, administration of said virus particle.
   648. The virus particle of embodiment 646 or embodiment 647, wherein the virus particle exhibits brain transduction that is at least 20-fold the brain transduction of a virus particle comprising capsid polypeptides of SEQ ID NO:1.
   649. The virus particle of embodiment 646 or embodiment 647, wherein the virus particle exhibits brain transduction that is at least 40-fold the brain transduction of a virus particle comprising capsid polypeptides of SEQ ID NO:1.
   650. The virus particle of embodiment 646 or embodiment 647, wherein the virus particle exhibits brain transduction that is at least 50-fold the brain transduction of a virus particle comprising capsid polypeptides of SEQ ID NO:1.
   651. The virus particle of embodiment 646 or embodiment 647, wherein the virus particle exhibits brain transduction that is at least 60-fold the brain transduction of a virus particle comprising capsid polypeptides of SEQ ID NO:1.
   652. The virus particle of embodiment 646 or embodiment 647, wherein the virus particle exhibits brain transduction that is at least 80-fold the brain transduction of a virus particle comprising capsid polypeptides of SEQ ID NO:1.
   653. The virus particle of embodiment 646 or embodiment 647, wherein the virus particle exhibits brain transduction that is at least 100-fold the brain transduction of a virus particle comprising capsid polypeptides of SEQ ID NO:1.
   654. The virus particle of embodiment 646 or embodiment 647, wherein the virus particle exhibits brain transduction that is at least 150-fold the brain transduction of a virus particle comprising capsid polypeptides of SEQ ID NO:1.
   655. The virus particle of embodiment 646 or embodiment 647, wherein the virus particle exhibits brain transduction that is at least 200-fold the brain transduction of a virus particle comprising capsid polypeptides of SEQ ID NO:1.
   656. The virus particle of any one of embodiments 646 to 649, wherein the virus particle exhibits brain transduction that is up to 60-fold the brain transduction of a virus particle comprising capsid polypeptides of SEQ ID NO:1.
   657. The virus particle of any one of embodiments 646 to 651, wherein the virus particle exhibits brain transduction that is up to 80-fold the brain transduction of a virus particle comprising capsid polypeptides of SEQ ID NO:1.
   658. The virus particle of any one of embodiments 646 to 652, wherein the virus particle exhibits brain transduction that is up to 100-fold the brain transduction of a virus particle comprising capsid polypeptides of SEQ ID NO:1.
   659. The virus particle of any one of embodiments 646 to 653, wherein the virus particle exhibits brain transduction that is up to 120-fold the brain transduction of a virus particle comprising capsid polypeptides of SEQ ID NO:1.
   660. The virus particle of any one of embodiments 646 to 653, wherein the virus particle exhibits brain transduction that is up to 150-fold the brain transduction of a virus particle comprising capsid polypeptides of SEQ ID NO:1.
   661. The virus particle of any one of embodiments 646 to 654, wherein the virus particle exhibits brain transduction that is up to 200-fold the brain transduction of a virus particle comprising capsid polypeptides of SEQ ID NO:1.
   662. The virus particle of any one of embodiments 646 to 655, wherein the virus particle exhibits brain transduction that is up to 225-fold the brain transduction of a virus particle comprising capsid polypeptides of SEQ ID NO:1.
   663. The virus particle of any one of embodiments 552 to 662, wherein said virus particle exhibits decreased DRG transduction, e.g., as measured in a mammal, e.g., in mouse or in NHP, e.g., as described herein, relative to wild-type AAV9 (e.g., a virus particle comprising capsid polypeptides of SEQ ID NO:1 or encoded by SEQ ID NO:2), optionally wherein the decreased PNS, e.g., decreased DRG transduction, (a) is in an amount described in Section 5.4, e.g., as set forth in any one of embodiments C-1 to C-22 and/or (b) is measured using a method described in Section 5.4.
   664. The virus particle of embodiment 663, wherein the decreased DRG transduction is exhibited upon systemic, e.g., intravenous, administration of said virus particle.
   665. The virus particle of embodiment 663 or embodiment 664, wherein the virus particle exhibits DRG transduction that is less than 1-fold the DRG transduction of a virus particle comprising capsid polypeptides of SEQ ID NO:1.
   666. The virus particle of embodiment 663 or embodiment 664, wherein the virus particle exhibits DRG transduction that is less than 0.1-fold the DRG transduction of a virus particle comprising capsid polypeptides of SEQ ID NO:1.
   667. The virus particle of embodiment 663 or embodiment 664, wherein the virus particle exhibits DRG transduction that is less than 0.05-fold the DRG transduction of a virus particle comprising capsid polypeptides of SEQ ID NO:1.
   668. The virus particle of embodiment 663 or embodiment 664, wherein the virus particle exhibits DRG transduction that is less than 0.03-fold the DRG transduction of a virus particle comprising capsid polypeptides of SEQ ID NO:1.
   669. The virus particle of embodiment 663 or embodiment 664, wherein the virus particle exhibits DRG transduction that is less than 0.02-fold the DRG transduction of a virus particle comprising capsid polypeptides of SEQ ID NO:1.
   670. The virus particle of any one of embodiments 663 to 669, wherein the virus particle exhibits DRG transduction that is at least 0.01-fold the DRG transduction of a virus particle comprising capsid polypeptides of SEQ ID NO:1.
   671. The virus particle of any one of embodiments 663 to 669, wherein the virus particle exhibits DRG transduction that is at least 0.015-fold the DRG transduction of a virus particle comprising capsid polypeptides of SEQ ID NO:1.
   672. The virus particle of any one of embodiments 663 to 669, wherein the virus particle exhibits DRG transduction that is at least 0.016-fold the DRG transduction of a virus particle comprising capsid polypeptides of SEQ ID NO:1.
   673. The virus particle of any one of embodiments 552 to 672, wherein said virus particle exhibits decreased liver transduction, e.g., as measured in a mammal, e.g., in mouse or in NHP, e.g., as described herein, relative to wild-type AAV9 (e.g., a virus particle comprising capsid polypeptides of SEQ ID NO:1 or encoded by SEQ ID NO:2), optionally wherein the decreased liver transduction (a) is in an amount described in Section 5.4, e.g., as set forth in any one of embodiments D-1 to D-23 and/or (b) is measured using a method described in Section 5.4.
   674. The virus particle of embodiment 673, wherein the decreased liver transduction is exhibited upon systemic, e.g., intravenous, administration of said virus particle.
   675. The virus particle of embodiment 673 or embodiment 674, wherein the virus particle exhibits liver transduction that is less than 1-fold the liver transduction of a virus particle comprising capsid polypeptides of SEQ ID NO:1.
   676. The virus particle of embodiment 673 or embodiment 674, wherein the virus particle exhibits liver transduction that is less than 0.9-fold the liver transduction of a virus particle comprising capsid polypeptides of SEQ ID NO:1.
   677. The virus particle of embodiment 673 or embodiment 674, wherein the virus particle exhibits liver transduction that is less than 0.8-fold the liver transduction of a virus particle comprising capsid polypeptides of SEQ ID NO:1.
   678. The virus particle of embodiment 673 or embodiment 674, wherein the virus particle exhibits liver transduction that is less than 0.7-fold the liver transduction of a virus particle comprising capsid polypeptides of SEQ ID NO:1.
   679. The virus particle of embodiment 673 or embodiment 674, wherein the virus particle exhibits liver transduction that is less than 0.6-fold the liver transduction of a virus particle comprising capsid polypeptides of SEQ ID NO:1.
   680. The virus particle of embodiment 673 or embodiment 674, wherein the virus particle exhibits liver transduction that is less than 0.5-fold the liver transduction of a virus particle comprising capsid polypeptides of SEQ ID NO:1.
   681. The virus particle of embodiment 673 or embodiment 674, wherein the virus particle exhibits liver transduction that is less than 0.4-fold the liver transduction of a virus particle comprising capsid polypeptides of SEQ ID NO:1.
   682. The virus particle of embodiment 673 or embodiment 674, wherein the virus particle exhibits liver transduction that is less than 0.3-fold the liver transduction of a virus particle comprising capsid polypeptides of SEQ ID NO:1.
   683. The virus particle of embodiment 673 or embodiment 674, wherein the virus particle exhibits liver transduction that is less than 0.2-fold the liver transduction of a virus particle comprising capsid polypeptides of SEQ ID NO:1.
   684. The virus particle of embodiment 673 or embodiment 674, wherein the virus particle exhibits liver transduction that is less than 0.1-fold the liver transduction of a virus particle comprising capsid polypeptides of SEQ ID NO:1.
   685. The virus particle of any one of embodiments 673 to 684, wherein the virus particle exhibits liver transduction that is at least 0.01-fold the liver transduction of a virus particle comprising capsid polypeptides of SEQ ID NO:1.
   686. The virus particle of any one of embodiments 673 to 684, wherein the virus particle exhibits liver transduction that is at least 0.03-fold the liver transduction of a virus particle comprising capsid polypeptides of SEQ ID NO:1.
   687. The virus particle of any one of embodiments 673 to 684, wherein the virus particle exhibits liver transduction that is at least 0.05-fold the liver transduction of a virus particle comprising capsid polypeptides of SEQ ID NO:1.
   688. The virus particle of any one of embodiments 673 to 683, wherein the virus particle exhibits liver transduction that is at least 0.1-fold the liver transduction of a virus particle comprising capsid polypeptides of SEQ ID NO:1.
   689. The virus particle of any one of embodiments 673 to 682, wherein the virus particle exhibits liver transduction that is at least 0.2-fold the liver transduction of a virus particle comprising capsid polypeptides of SEQ ID NO:1.
   690. The virus particle of any one of embodiments 673 to 681, wherein the virus particle exhibits liver transduction that is at least 0.3-fold the liver transduction of a virus particle comprising capsid polypeptides of SEQ ID NO:1.
   691. The virus particle of any one of embodiments 673 to 679, wherein the virus particle exhibits liver transduction that is at least 0.5-fold the liver transduction of a virus particle comprising capsid polypeptides of SEQ ID NO:1.
   692. The virus particle of any one of embodiments 673 to 677, wherein the virus particle exhibits liver transduction that is at least 0.7-fold the liver transduction of a virus particle comprising capsid polypeptides of SEQ ID NO:1.
   693. The virus particle of any one of embodiments 552 to 692, wherein said virus particle exhibits decreased spleen transduction, e.g., as measured in a mammal, e.g., in mouse or in NHP, e.g., as described herein, relative to wild-type AAV9 (e.g., a virus particle comprising capsid polypeptides of SEQ ID NO:1 or encoded by SEQ ID NO:2), optionally wherein the decreased spleen transduction (a) is in an amount described in Section 5.4, e.g., as set forth in any one of embodiments E-1 to E-22 and/or (b) is measured using a method described in Section 5.4.
   694. The virus particle of embodiment 693, wherein the decreased spleen transduction is exhibited upon systemic, e.g., intravenous, administration of said virus particle.
   695. The virus particle of embodiment 693 or embodiment 694, wherein the virus particle exhibits spleen transduction that is less than 1-fold the spleen transduction of a virus particle comprising capsid polypeptides of SEQ ID NO:1.
   696. The virus particle of embodiment 693 or embodiment 694, wherein the virus particle exhibits spleen transduction that is less than 0.9-fold the spleen transduction of a virus particle comprising capsid polypeptides of SEQ ID NO:1.
   697. The virus particle of embodiment 693 or embodiment 694, wherein the virus particle exhibits spleen transduction that is less than 0.8-fold the spleen transduction of a virus particle comprising capsid polypeptides of SEQ ID NO:1.
   698. The virus particle of embodiment 693 or embodiment 694, wherein the virus particle exhibits spleen transduction that is less than 0.7-fold the spleen transduction of a virus particle comprising capsid polypeptides of SEQ ID NO:1.
   699. The virus particle of embodiment 693 or embodiment 694, wherein the virus particle exhibits spleen transduction that is less than 0.6-fold the spleen transduction of a virus particle comprising capsid polypeptides of SEQ ID NO:1.
   700. The virus particle of embodiment 693 or embodiment 694, wherein the virus particle exhibits spleen transduction that is less than 0.5-fold the spleen transduction of a virus particle comprising capsid polypeptides of SEQ ID NO:1.
   701. The virus particle of embodiment 693 or embodiment 694, wherein the virus particle exhibits spleen transduction that is less than 0.4-fold the spleen transduction of a virus particle comprising capsid polypeptides of SEQ ID NO:1.
   702. The virus particle of embodiment 693 or embodiment 694, wherein the virus particle exhibits spleen transduction that is less than 0.3-fold the spleen transduction of a virus particle comprising capsid polypeptides of SEQ ID NO:1.
   703. The virus particle of embodiment 693 or embodiment 694, wherein the virus particle exhibits spleen transduction that is less than 0.2-fold the spleen transduction of a virus particle comprising capsid polypeptides of SEQ ID NO:1.
   704. The virus particle of embodiment 693 or embodiment 694, wherein the virus particle exhibits spleen transduction that is less than 0.1-fold the spleen transduction of a virus particle comprising capsid polypeptides of SEQ ID NO:1.
   705. The virus particle of embodiment 693 or embodiment 694, wherein the virus particle exhibits spleen transduction that is less than 0.05-fold the spleen transduction of a virus particle comprising capsid polypeptides of SEQ ID NO:1.
   706. The virus particle of embodiment 693 or embodiment 694, wherein the virus particle exhibits spleen transduction that is less than 0.02-fold the spleen transduction of a virus particle comprising capsid polypeptides of SEQ ID NO:1.
   707. The virus particle of any one of embodiments 693 to 706, wherein the virus particle exhibits spleen transduction that is at least 0.01-fold the spleen transduction of a virus particle comprising capsid polypeptides of SEQ ID NO:1.
   708. The virus particle of any one of embodiments 693 to 705, wherein the virus particle exhibits spleen transduction that is at least 0.02-fold the spleen transduction of a virus particle comprising capsid polypeptides of SEQ ID NO:1.
   709. The virus particle of any one of embodiments 552 to 708, wherein said virus particle exhibits production efficiency, e.g., as described herein, that is at least 0.1-fold the production efficiency of wild-type AAV9 (e.g., a virus particle comprising capsid polypeptides of SEQ ID NO:1 or encoded by SEQ ID NO:2).
   710. The virus particle of embodiment 709, wherein production efficiency of the virus particle is at least 0.2-fold the production efficiency of wild-type AAV9 (e.g., a virus particle comprising capsid polypeptides of SEQ ID NO:1 or encoded by SEQ ID NO:2).
   711. The virus particle of embodiment 709, wherein production efficiency of the virus particle is at least 0.3-fold the production efficiency of wild-type AAV9 (e.g., a virus particle comprising capsid polypeptides of SEQ ID NO:1 or encoded by SEQ ID NO:2).
   712. The virus particle of embodiment 709, wherein production efficiency of the virus particle is at least 0.4-fold the production efficiency of wild-type AAV9 (e.g., a virus particle comprising capsid polypeptides of SEQ ID NO:1 or encoded by SEQ ID NO:2).
   713. The virus particle of embodiment 709, wherein production efficiency of the virus particle is at least 0.5-fold the production efficiency of wild-type AAV9 (e.g., a virus particle comprising capsid polypeptides of SEQ ID NO:1 or encoded by SEQ ID NO:2).
   714. The virus particle of embodiment 709, wherein production efficiency of the virus particle is at least 0.6-fold the production efficiency of wild-type AAV9 (e.g., a virus particle comprising capsid polypeptides of SEQ ID NO:1 or encoded by SEQ ID NO:2).
   715. The virus particle of embodiment 709, wherein production efficiency of the virus particle is at least 0.7-fold the production efficiency of wild-type AAV9 (e.g., a virus particle comprising capsid polypeptides of SEQ ID NO:1 or encoded by SEQ ID NO:2).
   716. The virus particle of embodiment 709, wherein production efficiency of the virus particle is at least 0.8-fold the production efficiency of wild-type AAV9 (e.g., a virus particle comprising capsid polypeptides of SEQ ID NO:1 or encoded by SEQ ID NO:2).
   717. The virus particle of embodiment 709, wherein production efficiency of the virus particle is at least 0.9-fold the production efficiency of wild-type AAV9 (e.g., a virus particle comprising capsid polypeptides of SEQ ID NO:1 or encoded by SEQ ID NO:2).
   718. The virus particle of any one of embodiments 709 to 717, wherein production efficiency of the virus particle is up to 1-fold the production efficiency of wild-type AAV9 (e.g., a virus particle comprising capsid polypeptides of SEQ ID NO:1 or encoded by SEQ ID NO:2).
   719. The virus particle of any one of embodiments 552 to 718, wherein said virus particle exhibits increased skeletal muscle biodistribution, e.g., as measured in a mammal, e.g., in mouse or in NHP, e.g., as described herein, relative to wild-type AAV9 (e.g., a virus particle comprising capsid polypeptides of SEQ ID NO:1 or encoded by SEQ ID NO:2), optionally wherein the increased muscle biodistribution is (a) is in an amount described in Section 5.4, e.g., as set forth in any one of embodiments F-1 to F-17 and/or (b) is measured using a method described in Section 5.4.
   720. The virus particle of embodiment 719, increased skeletal muscle biodistribution is exhibited upon systemic, e.g., intravenous, administration of said virus particle.
   721. The virus particle of embodiment 719 or embodiment 720, wherein the virus particle exhibits skeletal muscle biodistribution that is at least 1.5-fold the skeletal muscle biodistribution of a virus particle comprising capsid polypeptides of SEQ ID NO:1.
   722. The virus particle of embodiment 721, wherein the virus particle exhibits skeletal muscle biodistribution that is at least 2-fold the skeletal muscle biodistribution of a virus particle comprising capsid polypeptides of SEQ ID NO:1.
   723. The virus particle of embodiment 721, wherein the virus particle exhibits skeletal muscle biodistribution that is at least 3-fold the skeletal muscle biodistribution of a virus particle comprising capsid polypeptides of SEQ ID NO:1.
   724. The virus particle of embodiment 721, wherein the virus particle exhibits skeletal muscle biodistribution that is at least 4-fold the skeletal muscle biodistribution of a virus particle comprising capsid polypeptides of SEQ ID NO:1.
   725. The virus particle of embodiment 721, wherein the virus particle exhibits skeletal muscle biodistribution that is at least 5-fold the skeletal muscle biodistribution of a virus particle comprising capsid polypeptides of SEQ ID NO:1, optionally wherein the skeletal muscle biodistribution is;
(a) at least 6-fold the skeletal muscle biodistribution of a virus particle comprising capsid polypeptides of SEQ ID NO:1;
(b) at least 7-fold the skeletal muscle biodistribution of a virus particle comprising capsid polypeptides of SEQ ID NO:1;
(c) at least 8-fold the skeletal muscle biodistribution of a virus particle comprising capsid polypeptides of SEQ ID NO:1; or
(d) at least 9-fold the skeletal muscle biodistribution of a virus particle comprising capsid polypeptides of SEQ ID NO:1.
   726. The virus particle of embodiment 721 or embodiment 725, wherein the virus particle exhibits skeletal muscle biodistribution that is 1.5-fold to 10-fold the skeletal muscle biodistribution of a virus particle comprising capsid polypeptides of SEQ ID NO:1.
   727. The virus particle of embodiment 721, wherein the virus particle exhibits skeletal muscle biodistribution that is 2-fold to 10-fold the skeletal muscle biodistribution of a virus particle comprising capsid polypeptides of SEQ ID NO:1.
   728. The virus particle of embodiment 721, wherein the virus particle exhibits skeletal muscle biodistribution that is 3-fold to 10-fold the skeletal muscle biodistribution of a virus particle comprising capsid polypeptides of SEQ ID NO:1.
   729. The virus particle of embodiment 721, wherein the virus particle exhibits skeletal muscle biodistribution that is 5-fold to 10-fold the skeletal muscle biodistribution of a virus particle comprising capsid polypeptides of SEQ ID NO:1.
   730. The virus particle of any one of embodiments 552 to 729, wherein said virus particle exhibits increased skeletal muscle transduction, e.g., as measured in a mammal, e.g., in mouse or in NHP, e.g., as described herein, relative to wild-type AAV9 (e.g., a virus particle comprising capsid polypeptides of SEQ ID NO:1 or encoded by SEQ ID NO:2), optionally wherein the increased muscle biodistribution is (a) is in an amount described in Section 5.4, e.g., as set forth in any one of embodiments G-1 to G-9 and/or (b) is measured using a method described in Section 5.4.
   731. The virus particle of embodiment 730, wherein increased skeletal muscle transduction is exhibited upon systemic, e.g., intravenous, administration of said virus particle.
   732. The virus particle of embodiment 730 or embodiment 731, wherein the virus particle exhibits skeletal muscle transduction that is at least 1.5-fold the skeletal muscle transduction of a virus particle comprising capsid polypeptides of SEQ ID NO:1.
   733. The virus particle of embodiment 732, wherein the virus particle exhibits skeletal muscle transduction that is at least 2-fold the skeletal muscle transduction of a virus particle comprising capsid polypeptides of SEQ ID NO:1.
   734. The virus particle of embodiment 732, wherein the virus particle exhibits skeletal muscle transduction that is at least 3-fold the skeletal muscle transduction of a virus particle comprising capsid polypeptides of SEQ ID NO:1.
   735. The virus particle of embodiment 732, wherein the virus particle exhibits skeletal muscle transduction that is at least 4-fold the skeletal muscle transduction of a virus particle comprising capsid polypeptides of SEQ ID NO:1.
   736. The virus particle of embodiment 721, wherein the virus particle exhibits skeletal muscle transduction that is 1.5-fold to 5-fold the skeletal muscle transduction of a virus particle comprising capsid polypeptides of SEQ ID NO:1.
   737. A method of producing a virus particle comprising a capsid polypeptide, said method comprising introducing a nucleic acid molecule of any one of embodiments 532 to 551 into a cell (e.g., a HEK293 cell), and harvesting said virus particles therefrom.
   738. A method of producing a virus particle comprising a capsid polypeptide, comprising culturing a cell engineered to express a capsid polypeptide of any one of embodiments 1 to 531 and harvesting virus particles therefrom, optionally wherein the cell is a host is a host cell described in any one of embodiments 1816 to 1826.
   739. A method of delivering a payload (e.g., a nucleic acid) to a cell comprising contacting the cell with a virus particle comprising the capsid polypeptide of any one of embodiments 1 to 531 and a payload, or contacting the cell with the virus particle of any one of embodiments 552 to 736.
   740. The method of embodiment 739, wherein the cell is a CNS cell.
   741. The method of embodiment 740, wherein the CNS cell is a neuronal cell, a glial cell, an astrocyte, an oligodendrocyte, an endothelial cell, or any combination thereof.
   742. The method of embodiment 739, wherein the cell is a skeletal muscle cell.
   743. A method of delivering a payload (e.g., a nucleic acid) to a subject comprising administering to the subject a virus particle comprising the capsid polypeptide of any one of embodiments 1 to 531 and the payload, or administering to the subject the virus particle of any one of embodiments 552 to 736.
   744. The method of embodiment 743, wherein the virus particle delivers the payload to the CNS.
   745. The method of embodiment 743 or embodiment 744, wherein the virus particle delivers the payload to skeletal muscle.
   746. The capsid polypeptide of any one of embodiments 1 to 531, the virus particle of any one of embodiments 552 to 736, or the method of any one of embodiments 737 to 744, wherein the virus particle (e.g., the virus particle comprising the capsid polypeptide) delivers the payload to the CNS with:
(a) increased biodistribution and/or transduction, e.g., biodistribution and/or transduction as compared to a virus particle comprising capsid polypeptides of SEQ ID NO:1, optionally wherein the biodistribution is at least 10-times, at least 20-times, at least 32-times, at least 50-times, at least 75-times or greater than the biodistribution and/or transduction of a virus particle comprising capsid polypeptides of SEQ ID NO:1; and optionally
(b) reduced liver, spleen, or DRG (or any combination of two or all three of liver, spleen and DRG) biodistribution as compared to a virus particle comprising capsid polypeptides of SEQ ID NO:1, optionally wherein the biodistribution in the liver, spleen, or DRG is each independently selected from:
   (i) less than 1-time,
   (ii) 0.95-times or less,
   (iii) 0.9-times or less,
   (iv) 0.8-times or less; or
   (v) 0.7 times or less;
than liver, spleen, or DRG (or any combination of two or all three of liver, spleen and DRG) biodistribution of a virus particle comprising capsid polypeptides of SEQ ID NO:1.
   747. The capsid polypeptide, virus particle or method of embodiment 746, wherein the one or more cell of the CNS is selected from a neuronal cell, a glial cell, an astrocyte, an oligodendrocyte, an endothelial cell, or any combination thereof.
   748. A method of treating a disease or condition in a subject, comprising administering to the subject in an amount effective to treat the disease or condition:
(a) a virus particle:
   (i) comprising the capsid polypeptide of any one of embodiments 1 to 531 and a heterologous nucleic acid sequence encoding a therapeutic product suitable for treating the disease or condition;
   (ii) comprising a capsid polypeptide encoded by the nucleic acid molecule of any one of embodiments 532 to 551 and a heterologous nucleic acid sequence encoding a therapeutic product suitable for treating the disease or condition, or
   (iii) of any one of embodiments 552 to 736; or
(b) a composition, e.g., a pharmaceutical composition, comprising the virus particle of (a) and, optionally, a pharmaceutically acceptable carrier.
   749. The method of embodiment 748, wherein the disease or condition is a disease or condition of the CNS.
   750. The method of embodiment 748 or embodiment 749, wherein the disease or condition is selected from Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS—Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Angleman syndrome, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Bechet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors (including, but not limited to those that have metastasized to the brain, for example, metastatic breast cancer), Brain Aneurysm, Brain Injury, Brown-Sequard Syndrome, Bulbar palsy, Bulbospinal Muscular Atrophy, Cerebral Autosomal Dominant Arteriopathy with Sub-cortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Concentric sclerosis (Balb's sclerosis), Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Chronic progressive external ophtalmoplegia, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia-Multi-Infarct, Dementia —Semantic, Dementia-Subcortical, Dementia With Lewy Bodies, Demyelination diseases, Dentate Cerebellar Ataxia, Dentatorubral Atrophy, Dermatomyositis, Developmental Dyspraxia, Devic's Syndrome, Diabetic Neuropathy, Diffuse Sclerosis, Distal hereditary motor neuronopathies, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalomyelitis, Encephalopathy, Encephalopathy (familial infantile), Encephalotrigeminal Angiomatosis, Epilepsy, Epileptic Hemiplegia, Episodic ataxia, Erb's Palsy, Erb-Duchenne and Dejerine-Klumpke Palsies, Essential Tremor, Extrapontine Myelinolysis, Faber's disease, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Fragile X disease, Friedreich's Ataxia, Frontotemporal Dementia, Gaucher Disease, Generalized Gangliosidoses (GM1, GM2), Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barre Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hurler syndrome, Hydranencephaly, Hydrocephalus, Hydrocephalus —Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kluver-Bucy Syndrome, Korsakoff's Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lichtheim's disease, Lipid Storage Diseases, Lipoid Proteinosis, Lissencephaly, Locked-In Syndrome, Lou Gehrig's Disease, Lupus —Neurological Sequelae, Lyme Disease —Neurological Complications, Lysosomal storage disorders, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Mitochondrial DNA depletion syndromes, Moebius Syndrome, Monomelic Amyotrophy, Morvan Syndrome, Motor Neuron Diseases, Moyamoya Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia —Congenital, Myasthenia Gravis, Myelinoclastic Diffuse Sclerosis, Myelitis, Myoclonic Encephalopathy of Infants, Myoclonus, Myoclonus epilepsy, Myopathy, Myopathy—Congenital, Myopathy-Thyrotoxic, Myotonia, Myotonia Congenita, Narcolepsy, NARP (neuropathy, ataxia and retinitis pigmentosa), Neuroacanthocytosis, Neurodegeneration with Brain Iron Accumulation, Neurodegenerative disease, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathic pain, Neuropathy—Hereditary, Neuropathy, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain-Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Peroneal muscular atrophy, Periodic Paralyses, Peripheral Neuropathy, Periventricular Leukomalacia, Persistent Vegetative State, Pervasive Developmental Disorders, Phelan McDermid syndrome, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive bulbar palsy, Progressive Hemifacial Atrophy, Progressive Locomotor Ataxia, Progressive Multifocal Leukoencephalopathy, Progressive Muscular Atrophy, Progressive Sclerosing Poliodystrophy, Progressive Supranuclear Palsy, Prosopagnosia, Pseudobulbar palsy, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjogren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Ataxia, Spinocerebellar Atrophy, Spinocerebellar Degeneration, Sporadic ataxia, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Vitamin B12 deficiency, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, and X-Linked Spinal or Bulbar Muscular Atrophy.
   751. The method of embodiment 749, wherein the disease or condition is Acid Lipase Disease.
   752. The method of embodiment 749, wherein the disease or condition is Acid Maltase Deficiency.
   753. The method of embodiment 749, wherein the disease or condition is Acquired Epileptiform Aphasia.
   754. The method of embodiment 749, wherein the disease or condition is Acute Disseminated Encephalomyelitis.
   755. The method of embodiment 749, wherein the disease or condition is Attention Deficit-Hyperactivity Disorder (ADHD).
   756. The method of embodiment 749, wherein the disease or condition is Adie's Pupil.
   757. The method of embodiment 749, wherein the disease or condition is Adie's Syndrome.
   758. The method of embodiment 749, wherein the disease or condition is Adrenoleukodystrophy.
   759. The method of embodiment 749, wherein the disease or condition is Agenesis of the Corpus Callosum.
   760. The method of embodiment 749, wherein the disease or condition is Agnosia.
   761. The method of embodiment 749, wherein the disease or condition is Aicardi Syndrome.
   762. The method of embodiment 749, wherein the disease or condition is Aicardi-Goutieres Syndrome Disorder.
   763. The method of embodiment 749, wherein the disease or condition is AIDS—Neurological Complications.
   764. The method of embodiment 749, wherein the disease or condition is Alexander Disease.
   765. The method of embodiment 749, wherein the disease or condition is Alpers' Disease.
   766. The method of embodiment 749, wherein the disease or condition is Alternating Hemiplegia.
   767. The method of embodiment 749, wherein the disease or condition is Alzheimer's Disease.
   768. The method of embodiment 749, wherein the disease or condition is Amyotrophic Lateral Sclerosis (ALS).
   769. The method of embodiment 749, wherein the disease or condition is Anencephaly.
   770. The method of embodiment 749, wherein the disease or condition is Aneurysm.
   771. The method of embodiment 749, wherein the disease or condition is Angelman Syndrome.
   772. The method of embodiment 749, wherein the disease or condition is Angiomatosis.
   773. The method of embodiment 749, wherein the disease or condition is Angleman syndrome.
   774. The method of embodiment 749, wherein the disease or condition is Anoxia.
   775. The method of embodiment 749, wherein the disease or condition is Antiphospholipid Syndrome.
   776. The method of embodiment 749, wherein the disease or condition is Aphasia.
   777. The method of embodiment 749, wherein the disease or condition is Apraxia.
   778. The method of embodiment 749, wherein the disease or condition is Arachnoid Cysts.
   779. The method of embodiment 749, wherein the disease or condition is Arachnoiditis.
   780. The method of embodiment 749, wherein the disease or condition is Arnold-Chiari Malformation.
   781. The method of embodiment 749, wherein the disease or condition is Arteriovenous Malformation.
   782. The method of embodiment 749, wherein the disease or condition is Asperger Syndrome.
   783. The method of embodiment 749, wherein the disease or condition is Ataxia.
   784. The method of embodiment 749, wherein the disease or condition is Ataxia Telangiectasia.
   785. The method of embodiment 749, wherein the disease or condition is Ataxia and Cerebellar or Spinocerebellar Degeneration.
   786. The method of embodiment 749, wherein the disease or condition is Atrial Fibrillation and Stroke.
   787. The method of embodiment 749, wherein the disease or condition is Attention Deficit-Hyperactivity Disorder.
   788. The method of embodiment 749, wherein the disease or condition is Autism Spectrum Disorder.
   789. The method of embodiment 749, wherein the disease or condition is Autonomic Dysfunction.
   790. The method of embodiment 749, wherein the disease or condition is Back Pain.
   791. The method of embodiment 749, wherein the disease or condition is Barth Syndrome.
   792. The method of embodiment 749, wherein the disease or condition is Batten Disease.
   793. The method of embodiment 749, wherein the disease or condition is Becker's Myotonia.
   794. The method of embodiment 749, wherein the disease or condition is Bechet's Disease.
   795. The method of embodiment 749, wherein the disease or condition is Bell's Palsy.
   796. The method of embodiment 749, wherein the disease or condition is Benign Essential Blepharospasm.
   797. The method of embodiment 749, wherein the disease or condition is Benign Focal Amyotrophy.
   798. The method of embodiment 749, wherein the disease or condition is Benign Intracranial Hypertension.
   799. The method of embodiment 749, wherein the disease or condition is Bernhardt-Roth Syndrome.
   800. The method of embodiment 749, wherein the disease or condition is Binswanger's Disease.
   801. The method of embodiment 749, wherein the disease or condition is Blepharospasm.
   802. The method of embodiment 749, wherein the disease or condition is Bloch-Sulzberger Syndrome.
   803. The method of embodiment 749, wherein the disease or condition is Brachial Plexus Birth Injury.
   804. The method of embodiment 749, wherein the disease or condition is Brachial Plexus Injury.
   805. The method of embodiment 749, wherein the disease or condition is Bradbury-Eggleston Syndrome.
   806. The method of embodiment 749, wherein the disease or condition is a Brain or Spinal Tumor (including, but not limited to a tumor that has metastasized to the brain, for example, metastatic breast cancer).
   807. The method of embodiment 749, wherein the disease or condition is Brain Aneurysm.
   808. The method of embodiment 749, wherein the disease or condition is Brain Injury.
   809. The method of embodiment 749, wherein the disease or condition is Brown-Sequard Syndrome.
   810. The method of embodiment 749, wherein the disease or condition is Bulbar palsy.
   811. The method of embodiment 749, wherein the disease or condition is Bulbospinal Muscular Atrophy.
   812. The method of embodiment 749, wherein the disease or condition is Cerebral Autosomal Dominant Arteriopathy with Sub-cortical Infarcts and Leukoencephalopathy (CADASIL).
   813. The method of embodiment 749, wherein the disease or condition is Canavan Disease.
   814. The method of embodiment 749, wherein the disease or condition is Carpal Tunnel Syndrome.
   815. The method of embodiment 749, wherein the disease or condition is Causalgia.
   816. The method of embodiment 749, wherein the disease or condition is Cavernomas.
   817. The method of embodiment 749, wherein the disease or condition is Cavernous Angioma.
   818. The method of embodiment 749, wherein the disease or condition is Cavernous Malformation.
   819. The method of embodiment 749, wherein the disease or condition is Central Cervical Cord Syndrome.
   820. The method of embodiment 749, wherein the disease or condition is Central Cord Syndrome.
   821. The method of embodiment 749, wherein the disease or condition is Central Pain Syndrome.
   822. The method of embodiment 749, wherein the disease or condition is Central Pontine Myelinolysis.
   823. The method of embodiment 749, wherein the disease or condition is a Cephalic Disorder.
   824. The method of embodiment 749, wherein the disease or condition is Ceramidase Deficiency.
   825. The method of embodiment 749, wherein the disease or condition is Cerebellar Degeneration.
   826. The method of embodiment 749, wherein the disease or condition is Cerebellar Hypoplasia.
   827. The method of embodiment 749, wherein the disease or condition is a Cerebral Aneurysm.
   828. The method of embodiment 749, wherein the disease or condition is Cerebral Arteriosclerosis.
   829. The method of embodiment 749, wherein the disease or condition is Cerebral Atrophy.
   830. The method of embodiment 749, wherein the disease or condition is Cerebral Beriberi.
   831. The method of embodiment 749, wherein the disease or condition is Cerebral Cavernous Malformation.
   832. The method of embodiment 749, wherein the disease or condition is Cerebral Gigantism.
   833. The method of embodiment 749, wherein the disease or condition is Cerebral Hypoxia.
   834. The method of embodiment 749, wherein the disease or condition is Cerebral Palsy.
   835. The method of embodiment 749, wherein the disease or condition is Cerebro-Oculo-Facio-Skeletal Syndrome (COFS).
   836. The method of embodiment 749, wherein the disease or condition is Charcot-Marie-Tooth Disease.
   837. The method of embodiment 749, wherein the disease or condition is Chiari Malformation.
   838. The method of embodiment 749, wherein the disease or condition is Cholesterol Ester Storage Disease.
   839. The method of embodiment 749, wherein the disease or condition is Chorea.
   840. The method of embodiment 749, wherein the disease or condition is Choreoacanthocytosis.
   841. The method of embodiment 749, wherein the disease or condition is Chronic Inflammatory Demyelinating Polyneuropathy (CIDP).
   842. The method of embodiment 749, wherein the disease or condition is Chronic Orthostatic Intolerance.
   843. The method of embodiment 749, wherein the disease or condition is Chronic Pain.
   844. The method of embodiment 749, wherein the disease or condition is Cockayne Syndrome Type II.
   845. The method of embodiment 749, wherein the disease or condition is Coffin Lowry Syndrome.
   846. The method of embodiment 749, wherein the disease or condition is Colpocephaly.
   847. The method of embodiment 749, wherein the disease or condition is Coma.
   848. The method of embodiment 749, wherein the disease or condition is Complex Regional Pain Syndrome.
   849. The method of embodiment 749, wherein the disease or condition is Concentric sclerosis (Bal6's sclerosis).
   850. The method of embodiment 749, wherein the disease or condition is Congenital Facial Diplegia.
   851. The method of embodiment 749, wherein the disease or condition is Congenital Myasthenia.
   852. The method of embodiment 749, wherein the disease or condition is Congenital Myopathy.
   853. The method of embodiment 749, wherein the disease or condition is Congenital Vascular Cavernous Malformations.
   854. The method of embodiment 749, wherein the disease or condition is Corticobasal Degeneration.
   855. The method of embodiment 749, wherein the disease or condition is Cranial Arteritis.
   856. The method of embodiment 749, wherein the disease or condition is Craniosynostosis.
   857. The method of embodiment 749, wherein the disease or condition is Cree encephalitis.
   858. The method of embodiment 749, wherein the disease or condition is Creutzfeldt-Jakob Disease.
   859. The method of embodiment 749, wherein the disease or condition is Chronic progressive external ophtalmoplegia.
   860. The method of embodiment 749, wherein the disease or condition is a Cumulative Trauma Disorder.
   861. The method of embodiment 749, wherein the disease or condition is Cushing's Syndrome.
   862. The method of embodiment 749, wherein the disease or condition is Cytomegalic Inclusion Body Disease.
   863. The method of embodiment 749, wherein the disease or condition is Cytomegalovirus Infection.
   864. The method of embodiment 749, wherein the disease or condition is Dancing Eyes-Dancing Feet Syndrome.
   865. The method of embodiment 749, wherein the disease or condition is Dandy-Walker Syndrome.
   866. The method of embodiment 749, wherein the disease or condition is Dawson Disease.
   867. The method of embodiment 749, wherein the disease or condition is De Morsier's Syndrome.
   868. The method of embodiment 749, wherein the disease or condition is Dejerine-Klumpke Palsy.
   869. The method of embodiment 749, wherein the disease or condition is Dementia.
   870. The method of embodiment 749, wherein the disease or condition is Dementia-Multi-Infarct.
   871. The method of embodiment 749, wherein the disease or condition is Dementia —Semantic.
   872. The method of embodiment 749, wherein the disease or condition is Dementia-Subcortical.
   873. The method of embodiment 749, wherein the disease or condition is Dementia With Lewy Bodies.
   874. The method of embodiment 749, wherein the disease or condition is a Demyelination disease.
   875. The method of embodiment 749, wherein the disease or condition is Dentate Cerebellar Ataxia.
   876. The method of embodiment 749, wherein the disease or condition is Dentatorubral Atrophy.
   877. The method of embodiment 749, wherein the disease or condition is Dermatomyositis.
   878. The method of embodiment 749, wherein the disease or condition is Developmental Dyspraxia.
   879. The method of embodiment 749, wherein the disease or condition is Devic's Syndrome.
   880. The method of embodiment 749, wherein the disease or condition is Diabetic Neuropathy.
   881. The method of embodiment 749, wherein the disease or condition is Diffuse Sclerosis.
   882. The method of embodiment 749, wherein the disease or condition is a Distal hereditary motor neuronopathy.
   883. The method of embodiment 749, wherein the disease or condition is Dravet Syndrome.
   884. The method of embodiment 749, wherein the disease or condition is Dysautonomia.
   885. The method of embodiment 749, wherein the disease or condition is Dysgraphia.
   886. The method of embodiment 749, wherein the disease or condition is Dyslexia.
   887. The method of embodiment 749, wherein the disease or condition is Dysphagia.
   888. The method of embodiment 749, wherein the disease or condition is Dyspraxia.
   889. The method of embodiment 749, wherein the disease or condition is Dyssynergia Cerebellaris Myoclonica.
   890. The method of embodiment 747, wherein the disease or condition is Dyssynergia Cerebellaris Progressiva.
   891. The method of embodiment 749, wherein the disease or condition is Dystonias.
   892. The method of embodiment 749, wherein the disease or condition is Early Infantile Epileptic Encephalopathy.
   893. The method of embodiment 749, wherein the disease or condition is Empty Sella Syndrome.
   894. The method of embodiment 749, wherein the disease or condition is Encephalitis.
   895. The method of embodiment 749, wherein the disease or condition is Encephalitis Lethargica.
   896. The method of embodiment 749, wherein the disease or condition is Encephaloceles.
   897. The method of embodiment 749, wherein the disease or condition is Encephalomyelitis.
   898. The method of embodiment 749, wherein the disease or condition is Encephalopathy.
   899. The method of embodiment 749, wherein the disease or condition is Encephalopathy (familial infantile).
   900. The method of embodiment 749, wherein the disease or condition is Encephalotrigeminal Angiomatosis.
   901. The method of embodiment 749, wherein the disease or condition is Epilepsy.
   902. The method of embodiment 749, wherein the disease or condition is Epileptic Hemiplegia.
   903. The method of embodiment 749, wherein the disease or condition is Episodic ataxia.
   904. The method of embodiment 749, wherein the disease or condition is Erb's Palsy.
   905. The method of embodiment 749, wherein the disease or condition is Erb-Duchenne Palsy
   906. The method of embodiment 749, wherein the disease or condition is Dejerine-Klumpke Palsy.
   907. The method of embodiment 749, wherein the disease or condition is Essential Tremor.
   908. The method of embodiment 749, wherein the disease or condition is Extrapontine Myelinolysis.
   909. The method of embodiment 749, wherein the disease or condition is Faber's disease.
   910. The method of embodiment 749, wherein the disease or condition is Fabry Disease.
   911. The method of embodiment 749, wherein the disease or condition is Fahr's Syndrome.
   912. The method of embodiment 749, wherein the disease or condition is Fainting.
   913. The method of embodiment 749, wherein the disease or condition is Familial Dysautonomia.
   914. The method of embodiment 749, wherein the disease or condition is Familial Hemangioma.
   915. The method of embodiment 749, wherein the disease or condition is Familial Idiopathic Basal Ganglia Calcification.
   916. The method of embodiment 749, wherein the disease or condition is Familial Periodic Paralyses.
   917. The method of embodiment 749, wherein the disease or condition is Familial Spastic Paralysis.
   918. The method of embodiment 749, wherein the disease or condition is Farber's Disease.
   919. The method of embodiment 749, wherein the disease or condition is Febrile Seizures.
   920. The method of embodiment 749, wherein the disease or condition is Fibromuscular Dysplasia.
   921. The method of embodiment 749, wherein the disease or condition is Fisher Syndrome.
   922. The method of embodiment 749, wherein the disease or condition is Floppy Infant Syndrome.
   923. The method of embodiment 749, wherein the disease or condition is Foot Drop.
   924. The method of embodiment 749, wherein the disease or condition is Fragile X disease.
   925. The method of embodiment 749, wherein the disease or condition is Friedreich's Ataxia.
   926. The method of embodiment 748, wherein the disease or condition is Frontotemporal Dementia.
   927. The method of embodiment 749, wherein the disease or condition is Gaucher Disease.
   928. The method of embodiment 749, wherein the disease or condition is Generalized Gangliosidoses (GM1, GM2).
   929. The method of embodiment 749, wherein the disease or condition is Gerstmann's Syndrome.
   930. The method of embodiment 749, wherein the disease or condition is Gerstmann-Straussler-Scheinker Disease.
   931. The method of embodiment 749, wherein the disease or condition is Giant Axonal Neuropathy.
   932. The method of embodiment 749, wherein the disease or condition is Giant Cell Arteritis.
   933. The method of embodiment 749, wherein the disease or condition is Giant Cell Inclusion Disease.
   934. The method of embodiment 749, wherein the disease or condition is Globoid Cell Leukodystrophy.
   935. The method of embodiment 749, wherein the disease or condition is Glossopharyngeal Neuralgia.
   936. The method of embodiment 749, wherein the disease or condition is Glycogen Storage Disease.
   937. The method of embodiment 749, wherein the disease or condition is Guillain-Barre Syndrome.
   938. The method of embodiment 749, wherein the disease or condition is Hallervorden-Spatz Disease.
   939. The method of embodiment 749, wherein the disease or condition is a Head Injury.
   940. The method of embodiment 749, wherein the disease or condition is Headache.
   941. The method of embodiment 749, wherein the disease or condition is Hemicrania Continua.
   942. The method of embodiment 749, wherein the disease or condition is Hemifacial Spasm.
   943. The method of embodiment 749, wherein the disease or condition is Hemiplegia Alterans.
   944. The method of embodiment 749, wherein the disease or condition is a Hereditary Neuropathy.
   945. The method of embodiment 749, wherein the disease or condition is Hereditary Spastic Paraplegia.
   946. The method of embodiment 749, wherein the disease or condition is Heredopathia Atactica Polyneuritiformis.
   947. The method of embodiment 749, wherein the disease or condition is Herpes Zoster.
   948. The method of embodiment 749, wherein the disease or condition is Herpes Zoster Oticus.
   949. The method of embodiment 749, wherein the disease or condition is Hirayama Syndrome.
   950. The method of embodiment 749, wherein the disease or condition is Holmes-Adie syndrome.
   951. The method of embodiment 749, wherein the disease or condition is Holoprosencephaly.
   952. The method of embodiment 749, wherein the disease or condition is HTLV-1 Associated Myelopathy.
   953. The method of embodiment 749, wherein the disease or condition is Hughes Syndrome.
   954. The method of embodiment 749, wherein the disease or condition is Huntington's Disease.
   955. The method of embodiment 749, wherein the disease or condition is Hurler syndrome.
   956. The method of embodiment 749, wherein the disease or condition is Hydranencephaly.
   957. The method of embodiment 749, wherein the disease or condition is Hydrocephalus.
   958. The method of embodiment 749, wherein the disease or condition is Hydrocephalus —Normal Pressure.
   959. The method of embodiment 749, wherein the disease or condition is Hydromyelia.
   960. The method of embodiment 749, wherein the disease or condition is Hypercortisolism.
   961. The method of embodiment 749, wherein the disease or condition is Hypersomnia.
   962. The method of embodiment 749, wherein the disease or condition is Hypertonia.
   963. The method of embodiment 749, wherein the disease or condition is Hypotonia.
   964. The method of embodiment 749, wherein the disease or condition is Hypoxia.
   965. The method of embodiment 749, wherein the disease or condition is Immune-Mediated Encephalomyelitis.
   966. The method of embodiment 749, wherein the disease or condition is Inclusion Body Myositis.
   967. The method of embodiment 749, wherein the disease or condition is Incontinentia Pigmenti.
   968. The method of embodiment 749, wherein the disease or condition is Infantile Hypotonia.
   969. The method of embodiment 749, wherein the disease or condition is Infantile Neuroaxonal Dystrophy.
   970. The method of embodiment 749, wherein the disease or condition is Infantile Phytanic Acid Storage Disease.
   971. The method of embodiment 749, wherein the disease or condition is Infantile Refsum Disease.
   972. The method of embodiment 749, wherein the disease or condition is Infantile Spasms.
   973. The method of embodiment 749, wherein the disease or condition is an Inflammatory Myopathy.
   974. The method of embodiment 749, wherein the disease or condition is Iniencephaly.
   975. The method of embodiment 749, wherein the disease or condition is Intestinal Lipodystrophy.
   976. The method of embodiment 749, wherein the disease or condition is Intracranial Cysts.
   977. The method of embodiment 749, wherein the disease or condition is Intracranial Hypertension.
   978. The method of embodiment 747, wherein the disease or condition is Isaacs' Syndrome.
   979. The method of embodiment 749, wherein the disease or condition is Joubert Syndrome.
   980. The method of embodiment 749, wherein the disease or condition is Kearns-Sayre Syndrome.
   981. The method of embodiment 749, wherein the disease or condition is Kennedy's Disease.
   982. The method of embodiment 749, wherein the disease or condition is Kinsbourne syndrome.
   983. The method of embodiment 749, wherein the disease or condition is Kleine-Levin Syndrome.
   984. The method of embodiment 749, wherein the disease or condition is Klippel-Feil Syndrome.
   985. The method of embodiment 749, wherein the disease or condition is Klippel-Trenaunay Syndrome (KTS).
   986. The method of embodiment 749, wherein the disease or condition is Kiuver-Bucy Syndrome.
   987. The method of embodiment 749, wherein the disease or condition is Korsakoff's Amnesic Syndrome.
   988. The method of embodiment 749, wherein the disease or condition is Krabbe Disease.
   989. The method of embodiment 749, wherein the disease or condition is Kugelberg-Welander Disease.
   990. The method of embodiment 749, wherein the disease or condition is Kuru.
   991. The method of embodiment 749, wherein the disease or condition is Lambert-Eaton Myasthenic Syndrome.
   992. The method of embodiment 749, wherein the disease or condition is Landau-Kleffner Syndrome.
   993. The method of embodiment 749, wherein the disease or condition is Lateral Femoral Cutaneous Nerve Entrapment.
   994. The method of embodiment 749, wherein the disease or condition is Lateral Medullary Syndrome.
   995. The method of embodiment 749, wherein the disease or condition is a Learning Disability.
   996. The method of embodiment 749, wherein the disease or condition is Leigh's Disease.
   997. The method of embodiment 749, wherein the disease or condition is Lennox-Gastaut Syndrome.
   998. The method of embodiment 749, wherein the disease or condition is Lesch-Nyhan Syndrome.
   999. The method of embodiment 749, wherein the disease or condition is Leukodystrophy.
   1000. The method of embodiment 749, wherein the disease or condition is Levine-Critchley Syndrome.
   1001. The method of embodiment 749, wherein the disease or condition is Lewy Body Dementia.
   1002. The method of embodiment 749, wherein the disease or condition is Lichtheim's disease.
   1003. The method of embodiment 749, wherein the disease or condition is a Lipid Storage Disease.
   1004. The method of embodiment 749, wherein the disease or condition is Lipoid Proteinosis.
   1005. The method of embodiment 749, wherein the disease or condition is Lissencephaly.
   1006. The method of embodiment 749, wherein the disease or condition is Locked-In Syndrome.
   1007. The method of embodiment 749, wherein the disease or condition is Lou Gehrig's Disease.
   1008. The method of embodiment 749, wherein the disease or condition is Lupus —Neurological Sequelae.
   1009. The method of embodiment 749, wherein the disease or condition is Lyme Disease-Neurological Complications.
   1010. The method of embodiment 749, wherein the disease or condition is Lysosomal storage disorders.
   1011. The method of embodiment 749, wherein the disease or condition is Machado-Joseph Disease.
   1012. The method of embodiment 749, wherein the disease or condition is Macrencephaly.
   1013. The method of embodiment 749, wherein the disease or condition is Megalencephaly.
   1014. The method of embodiment 749, wherein the disease or condition is Melkersson-Rosenthal Syndrome.
   1015. The method of embodiment 749, wherein the disease or condition is Meningitis.
   1016. The method of embodiment 749, wherein the disease or condition is Meningitis and Encephalitis.
   1017. The method of embodiment 749, wherein the disease or condition is Menkes Disease.
   1018. The method of embodiment 749, wherein the disease or condition is Meralgia Paresthetica.
   1019. The method of embodiment 749, wherein the disease or condition is Metachromatic Leukodystrophy.
   1020. The method of embodiment 749, wherein the disease or condition is Microcephaly.
   1021. The method of embodiment 749, wherein the disease or condition is Migraine.
   1022. The method of embodiment 749, wherein the disease or condition is Miller Fisher Syndrome.
   1023. The method of embodiment 749, wherein the disease or condition is Mini Stroke.
   1024. The method of embodiment 749, wherein the disease or condition is Mitochondrial Myopathy.
   1025. The method of embodiment 749, wherein the disease or condition is a Mitochondrial DNA depletion syndrome.
   1026. The method of embodiment 749, wherein the disease or condition is Moebius Syndrome.
   1027. The method of embodiment 749, wherein the disease or condition is Monomelic Amyotrophy.
   1028. The method of embodiment 749, wherein the disease or condition is Morvan Syndrome.
   1029. The method of embodiment 749, wherein the disease or condition is a Motor Neuron Disease.
   1030. The method of embodiment 749, wherein the disease or condition is Moyamoya Disease.
   1031. The method of embodiment 749, wherein the disease or condition is Mucolipidoses.
   1032. The method of embodiment 749, wherein the disease or condition is Mucopolysaccharidoses.
   1033. The method of embodiment 749, wherein the disease or condition is Multi-Infarct Dementia.
   1034. The method of embodiment 749, wherein the disease or condition is Multifocal Motor Neuropathy.
   1035. The method of embodiment 749, wherein the disease or condition is Multiple Sclerosis.
   1036. The method of embodiment 749, wherein the disease or condition is Multiple System Atrophy.
   1037. The method of embodiment 749, wherein the disease or condition is Multiple System Atrophy with Orthostatic Hypotension.
   1038. The method of embodiment 749, wherein the disease or condition is Muscular Dystrophy.
   1039. The method of embodiment 749, wherein the disease or condition is Myasthenia —Congenital.
   1040. The method of embodiment 749, wherein the disease or condition is Myasthenia Gravis.
   1041. The method of embodiment 749, wherein the disease or condition is Myelinoclastic Diffuse Sclerosis.
   1042. The method of embodiment 749, wherein the disease or condition is Myelitis.
   1043. The method of embodiment 749, wherein the disease or condition is Myoclonic Encephalopathy of Infants.
   1044. The method of embodiment 749, wherein the disease or condition is Myoclonus.
   1045. The method of embodiment 749, wherein the disease or condition is Myoclonus epilepsy.
   1046. The method of embodiment 749, wherein the disease or condition is Myopathy.
   1047. The method of embodiment 749, wherein the disease or condition is Myopathy—Congenital.
   1048. The method of embodiment 749, wherein the disease or condition is Myopathy-Thyrotoxic.
   1049. The method of embodiment 749, wherein the disease or condition is Myotonia.
   1050. The method of embodiment 749, wherein the disease or condition is Myotonia Congenita.
   1051. The method of embodiment 749, wherein the disease or condition is Narcolepsy.
   1052. The method of embodiment 749, wherein the disease or condition is NARP (neuropathy, ataxia and retinitis pigmentosa).
   1053. The method of embodiment 749, wherein the disease or condition is Neuroacanthocytosis.
   1054. The method of embodiment 749, wherein the disease or condition is Neurodegeneration with Brain Iron Accumulation.
   1055. The method of embodiment 749, wherein the disease or condition is a Neurodegenerative disease.
   1056. The method of embodiment 749, wherein the disease or condition is Neurofibromatosis.
   1057. The method of embodiment 749, wherein the disease or condition is Neuroleptic Malignant Syndrome.
   1058. The method of embodiment 749, wherein the disease or condition is Neurological Complications of AIDS.
   1059. The method of embodiment 749, wherein the disease or condition is Neurological Complications of Lyme Disease.
   1060. The method of embodiment 749, wherein the disease or condition is Neurological Consequences of Cytomegalovirus Infection.
   1061. The method of embodiment 749, wherein the disease or condition is Neurological Manifestations of Pompe Disease.
   1062. The method of embodiment 749, wherein the disease or condition is Neurological Sequelae Of Lupus.
   1063. The method of embodiment 749, wherein the disease or condition is Neuromyelitis Optica.
   1064. The method of embodiment 749, wherein the disease or condition is Neuromyotonia.
   1065. The method of embodiment 749, wherein the disease or condition is Neuronal Ceroid Lipofuscinosis.
   1066. The method of embodiment 749, wherein the disease or condition is Neuronal Migration Disorders.
   1067. The method of embodiment 749, wherein the disease or condition is Neuropathic pain.
   1068. The method of embodiment 749, wherein the disease or condition is Neuropathy—Hereditary.
   1069. The method of embodiment 749, wherein the disease or condition is Neuropathy.
   1070. The method of embodiment 749, wherein the disease or condition is Neurosarcoidosis.
   1071. The method of embodiment 749, wherein the disease or condition is Neurosyphilis.
   1072. The method of embodiment 749, wherein the disease or condition is Neurotoxicity.
   1073. The method of embodiment 749, wherein the disease or condition is Nevus Cavernosus.
   1074. The method of embodiment 749, wherein the disease or condition is Niemann-Pick Disease.
   1075. The method of embodiment 749, wherein the disease or condition is O'Sullivan-McLeod Syndrome.
   1076. The method of embodiment 749, wherein the disease or condition is Occipital Neuralgia.
   1077. The method of embodiment 749, wherein the disease or condition is Ohtahara Syndrome.
   1078. The method of embodiment 749, wherein the disease or condition is Olivopontocerebellar Atrophy.
   1079. The method of embodiment 749, wherein the disease or condition is Opsoclonus Myoclonus.
   1080. The method of embodiment 749, wherein the disease or condition is Orthostatic Hypotension.
   1081. The method of embodiment 749, wherein the disease or condition is Overuse Syndrome.
   1082. The method of embodiment 749, wherein the disease or condition is Pain-Chronic.
   1083. The method of embodiment 749, wherein the disease or condition is Pantothenate Kinase-Associated Neurodegeneration.
   1084. The method of embodiment 749, wherein the disease or condition is Paraneoplastic Syndromes.
   1085. The method of embodiment 749, wherein the disease or condition is Paresthesia.
   1086. The method of embodiment 749, wherein the disease or condition is Parkinson's Disease.
   1087. The method of embodiment 749, wherein the disease or condition is Paroxysmal Choreoathetosis.
   1088. The method of embodiment 749, wherein the disease or condition is Paroxysmal Hemicrania.
   1089. The method of embodiment 749, wherein the disease or condition is Parry-Romberg.
   1090. The method of embodiment 749, wherein the disease or condition is Pelizaeus-Merzbacher Disease.
   1091. The method of embodiment 749, wherein the disease or condition is Pena Shokeir II Syndrome.
   1092. The method of embodiment 749, wherein the disease or condition is Perineural Cysts.
   1093. The method of embodiment 749, wherein the disease or condition is Peroneal muscular atrophy.
   1094. The method of embodiment 749, wherein the disease or condition is Periodic Paralyses.
   1095. The method of embodiment 749, wherein the disease or condition is Peripheral Neuropathy.
   1096. The method of embodiment 749, wherein the disease or condition is Periventricular Leukomalacia.
   1097. The method of embodiment 749, wherein the disease or condition is Persistent Vegetative State.
   1098. The method of embodiment 749, wherein the disease or condition is a Pervasive Developmental Disorder.
   1099. The method of embodiment 749, wherein the disease or condition is Phelan McDermid syndrome.
   1100. The method of embodiment 749, wherein the disease or condition is Phytanic Acid Storage Disease.
   1101. The method of embodiment 749, wherein the disease or condition is Pick's Disease.
   1102. The method of embodiment 749, wherein the disease or condition is Pinched Nerve.
   1103. The method of embodiment 749, wherein the disease or condition is Piriformis Syndrome.
   1104. The method of embodiment 749, wherein the disease or condition is a Pituitary Tumor.
   1105. The method of embodiment 749, wherein the disease or condition is Polymyositis.
   1106. The method of embodiment 749, wherein the disease or condition is Pompe Disease.
   1107. The method of embodiment 749, wherein the disease or condition is Porencephaly.
   1108. The method of embodiment 749, wherein the disease or condition is Post-Polio Syndrome.
   1109. The method of embodiment 749, wherein the disease or condition is Postherpetic Neuralgia.
   1110. The method of embodiment 749, wherein the disease or condition is Postinfectious Encephalomyelitis.
   1111. The method of embodiment 749, wherein the disease or condition is Postural Hypotension.
   1112. The method of embodiment 749, wherein the disease or condition is Postural Orthostatic Tachycardia Syndrome.
   1113. The method of embodiment 749, wherein the disease or condition is Postural Tachycardia Syndrome.
   1114. The method of embodiment 749, wherein the disease or condition is Primary Dentatum Atrophy.
   1115. The method of embodiment 749, wherein the disease or condition is Primary Lateral Sclerosis.
   1116. The method of embodiment 749, wherein the disease or condition is Primary Progressive Aphasia.
   1117. The method of embodiment 749, wherein the disease or condition is a Prion Disease.
   1118. The method of embodiment 749, wherein the disease or condition is Progressive bulbar palsy.
   1119. The method of embodiment 749, wherein the disease or condition is Progressive Hemifacial Atrophy.
   1120. The method of embodiment 749, wherein the disease or condition is Progressive Locomotor Ataxia.
   1121. The method of embodiment 749, wherein the disease or condition is Progressive Multifocal Leukoencephalopathy.
   1122. The method of embodiment 749, wherein the disease or condition is Progressive Muscular Atrophy.
   1123. The method of embodiment 749, wherein the disease or condition is Progressive Sclerosing Poliodystrophy.
   1124. The method of embodiment 749, wherein the disease or condition is Progressive Supranuclear Palsy.
   1125. The method of embodiment 749, wherein the disease or condition is Prosopagnosia.
   1126. The method of embodiment 749, wherein the disease or condition is Pseudobulbar palsy.
   1127. The method of embodiment 749, wherein the disease or condition is Pseudo-Torch syndrome.
   1128. The method of embodiment 749, wherein the disease or condition is Pseudotoxoplasmosis syndrome.
   1129. The method of embodiment 749, wherein the disease or condition is Pseudotumor Cerebri.
   1130. The method of embodiment 749, wherein the disease or condition is Psychogenic Movement.
   1131. The method of embodiment 749, wherein the disease or condition is Ramsay Hunt Syndrome I.
   1132. The method of embodiment 749, wherein the disease or condition is Ramsay Hunt Syndrome II.
   1133. The method of embodiment 749, wherein the disease or condition is Rasmussen's Encephalitis.
   1134. The method of embodiment 749, wherein the disease or condition is Reflex Sympathetic Dystrophy Syndrome.
   1135. The method of embodiment 749, wherein the disease or condition is Refsum Disease.
   1136. The method of embodiment 749, wherein the disease or condition is Refsum Disease —Infantile.
   1137. The method of embodiment 749, wherein the disease or condition is a Repetitive Motion Disorder.
   1138. The method of embodiment 749, wherein the disease or condition is a Repetitive Stress Injury.
   1139. The method of embodiment 749, wherein the disease or condition is Restless Legs Syndrome.
   1140. The method of embodiment 749, wherein the disease or condition is Retrovirus-Associated Myelopathy.
   1141. The method of embodiment 749, wherein the disease or condition is Rett Syndrome.
   1142. The method of embodiment 749, wherein the disease or condition is Reye's Syndrome.
   1143. The method of embodiment 749, wherein the disease or condition is Rheumatic Encephalitis.
   1144. The method of embodiment 749, wherein the disease or condition is Riley-Day Syndrome.
   1145. The method of embodiment 749, wherein the disease or condition is Sacral Nerve Root Cysts.
   1146. The method of embodiment 749, wherein the disease or condition is Saint Vitus Dance.
   1147. The method of embodiment 749, wherein the disease or condition is Salivary Gland Disease.
   1148. The method of embodiment 749, wherein the disease or condition is Sandhoff Disease.
   1149. The method of embodiment 749, wherein the disease or condition is Schilder's Disease.
   1150. The method of embodiment 749, wherein the disease or condition is Schizencephaly.
   1151. The method of embodiment 749, wherein the disease or condition is Seitelberger Disease.
   1152. The method of embodiment 749, wherein the disease or condition is Seizure Disorder.
   1153. The method of embodiment 749, wherein the disease or condition is Semantic Dementia.
   1154. The method of embodiment 749, wherein the disease or condition is Septo-Optic Dysplasia.
   1155. The method of embodiment 749, wherein the disease or condition is Severe Myoclonic Epilepsy of Infancy (SMEI).
   1156. The method of embodiment 749, wherein the disease or condition is Shaken Baby Syndrome.
   1157. The method of embodiment 749, wherein the disease or condition is Shingles.
   1158. The method of embodiment 749, wherein the disease or condition is Shy-Drager Syndrome.
   1159. The method of embodiment 749, wherein the disease or condition is Sjogren's Syndrome.
   1160. The method of embodiment 749, wherein the disease or condition is Sleep Apnea.
   1161. The method of embodiment 749, wherein the disease or condition is Sleeping Sickness.
   1162. The method of embodiment 749, wherein the disease or condition is Sotos Syndrome.
   1163. The method of embodiment 749, wherein the disease or condition is Spasticity.
   1164. The method of embodiment 749, wherein the disease or condition is Spina Bifida.
   1165. The method of embodiment 749, wherein the disease or condition is Spinal Cord Infarction.
   1166. The method of embodiment 749, wherein the disease or condition is Spinal Cord Injury.
   1167. The method of embodiment 749, wherein the disease or condition is a Spinal Cord Tumor.
   1168. The method of embodiment 749, wherein the disease or condition is Spinal Muscular Atrophy.
   1169. The method of embodiment 749, wherein the disease or condition is Spinocerebellar Ataxia.
   1170. The method of embodiment 749, wherein the disease or condition is Spinocerebellar Atrophy.
   1171. The method of embodiment 749, wherein the disease or condition is Spinocerebellar Degeneration.
   1172. The method of embodiment 749, wherein the disease or condition is Sporadic ataxia.
   1173. The method of embodiment 749, wherein the disease or condition is Steele-Richardson-Olszewski Syndrome.
   1174. The method of embodiment 749, wherein the disease or condition is Stiff-Person Syndrome.
   1175. The method of embodiment 749, wherein the disease or condition is Striatonigral Degeneration.
   1176. The method of embodiment 749, wherein the disease or condition is Stroke.
   1177. The method of embodiment 749, wherein the disease or condition is Sturge-Weber Syndrome.
   1178. The method of embodiment 749, wherein the disease or condition is Subacute Sclerosing Panencephalitis.
   1179. The method of embodiment 749, wherein the disease or condition is Subcortical Arteriosclerotic Encephalopathy.
   1180. The method of embodiment 749, wherein the disease or condition is Short-lasting, Unilateral, Neuralgiform (SUNCT) Headache.
   1181. The method of embodiment 749, wherein the disease or condition is a Swallowing Disorder.
   1182. The method of embodiment 749, wherein the disease or condition is Sydenham Chorea.
   1183. The method of embodiment 749, wherein the disease or condition is Syncope.
   1184. The method of embodiment 749, wherein the disease or condition is Syphilitic Spinal Sclerosis.
   1185. The method of embodiment 749, wherein the disease or condition is Syringohydromyelia.
   1186. The method of embodiment 749, wherein the disease or condition is Syringomyelia.
   1187. The method of embodiment 749, wherein the disease or condition is Systemic Lupus Erythematosus.
   1188. The method of embodiment 749, wherein the disease or condition is Tabes Dorsalis.
   1189. The method of embodiment 749, wherein the disease or condition is Tardive Dyskinesia.
   1190. The method of embodiment 749, wherein the disease or condition is Tarlov Cysts.
   1191. The method of embodiment 749, wherein the disease or condition is Tay-Sachs Disease.
   1192. The method of embodiment 749, wherein the disease or condition is Temporal Arteritis.
   1193. The method of embodiment 749, wherein the disease or condition is Tethered Spinal Cord Syndrome.
   1194. The method of embodiment 749, wherein the disease or condition is Thomsen's Myotonia.
   1195. The method of embodiment 749, wherein the disease or condition is Thoracic Outlet Syndrome.
   1196. The method of embodiment 749, wherein the disease or condition is Thyrotoxic Myopathy.
   1197. The method of embodiment 749, wherein the disease or condition is Tic Douloureux.
   1198. The method of embodiment 749, wherein the disease or condition is Todd's Paralysis.
   1199. The method of embodiment 749, wherein the disease or condition is Tourette Syndrome.
   1200. The method of embodiment 749, wherein the disease or condition is Transient Ischemic Attack.
   1201. The method of embodiment 749, wherein the disease or condition is Transmissible Spongiform Encephalopathies.
   1202. The method of embodiment 749, wherein the disease or condition is Transverse Myelitis.
   1203. The method of embodiment 749, wherein the disease or condition is Traumatic Brain Injury.
   1204. The method of embodiment 749, wherein the disease or condition is Tremor.
   1205. The method of embodiment 749, wherein the disease or condition is Trigeminal Neuralgia.
   1206. The method of embodiment 749, wherein the disease or condition is Tropical Spastic Paraparesis.
   1207. The method of embodiment 749, wherein the disease or condition is Troyer Syndrome.
   1208. The method of embodiment 749, wherein the disease or condition is Tuberous Sclerosis.
   1209. The method of embodiment 749, wherein the disease or condition is Vascular Erectile Tumor.
   1210. The method of embodiment 749, wherein the disease or condition is a Vasculitis Syndrome of the Central or Peripheral Nervous System.
   1211. The method of embodiment 749, wherein the disease or condition is Vitamin B12 deficiency.
   1212. The method of embodiment 749, wherein the disease or condition is Von Economo's Disease.
   1213. The method of embodiment 749, wherein the disease or condition is Von Hippel-Lindau Disease (VHL).
   1214. The method of embodiment 749, wherein the disease or condition is Von Recklinghausen's Disease.
   1215. The method of embodiment 749, wherein the disease or condition is Wallenberg's Syndrome.
   1216. The method of embodiment 749, wherein the disease or condition is Werdnig-Hoffman Disease.
   1217. The method of embodiment 749, wherein the disease or condition is Wernicke-Korsakoff Syndrome.
   1218. The method of embodiment 749, wherein the disease or condition is West Syndrome.
   1219. The method of embodiment 749, wherein the disease or condition is Whiplash.
   1220. The method of embodiment 749, wherein the disease or condition is Whipple's Disease.
   1221. The method of embodiment 749, wherein the disease or condition is Williams Syndrome.
   1222. The method of embodiment 749, wherein the disease or condition is Wilson Disease.
   1223. The method of embodiment 749, wherein the disease or condition is Wolman's Disease.
   1224. The method of embodiment 749, wherein the disease or condition is X-Linked Spinal or Bulbar Muscular Atrophy.
   1225. The method of embodiment 748, wherein the disease or condition is a disease or condition of skeletal muscle.
   1226. The method of embodiment 748 or embodiment 1225, wherein the disease or condition of skeletal muscle is Acid Maltase Deficiency (AMD), Amyotrophic Lateral Sclerosis (ALS), Andersen-Tawil Syndrome, Barth syndrome (TAZ), Becker Muscular Dystrophy (BMD), Becker Myotonia Congenita, Bethlem Myopathy, Bulbospinal Muscular Atrophy (Spinal—Bulbar Muscular Atrophy), Carnitine Deficiency, Carnitine Palmityl Transferase Deficiency (CPT Deficiency), Central Core Disease (CCD), Centronuclear Myopathy, Charcot-Marie-Tooth Disease (CMT), Congenital Muscular Dystrophy (CMD), Congenital Myasthenic Syndromes (CMS), Congenital Myotonic Dystrophy, Cori Disease (Debrancher Enzyme Deficiency), Danon disease, Debrancher Enzyme Deficiency, Dejerine-Sottas Disease (DSD), Dermatomyositis (DM), Distal Muscular Dystrophy (DD), Distal myopathy with anterior tibial onset, Duchenne Muscular Dystrophy (DMD), Dystrophia Myotonica (Myotonic Muscular Dystrophy), Emery-Dreifuss Muscular Dystrophy (EDMD), Endocrine Myopathies, Eulenberg Disease (Paramyotonia Congenita), Facioscapulohumeral Muscular Dystrophy (FSH or FSHD), Finnish (Tibial) Distal Myopathy, Forbes Disease (Debrancher Enzyme Deficiency), Friedreich's Ataxia (FA), Fukuyama Congenital Muscular Dystrophy, Glycogenosis Type 10, Glycogenosis Type 11, Glycogenosis Type 2, Glycogenosis Type 3, Glycogenosis Type 5, Glycogenosis Type 7, Glycogenosis Type 9, Gowers-Laing Distal Myopathy, Hauptmann-Thanheuser MD (Emery-Dreifuss Muscular Dystrophy), Hereditary Inclusion-Body Myositis, Hereditary Motor and Sensory Neuropathy (Charcot-Marie-Tooth Disease), Hyperthyroid Myopathy, Hypothyroid Myopathy, Inclusion-Body Myositis (IBM), Inherited Myopathies, Integrin-Deficient Congenital Muscular Dystrophy, Kennedy Disease (Spinal-Bulbar Muscular Atrophy), Kugelberg-Welander Disease (Spinal Muscular Atrophy), Lactate Dehydrogenase Deficiency, Lambert-Eaton Myasthenic Syndrome (LEMS), Limb-Girdle Muscular Dystrophy (LGMD), Lou Gehrig's Disease (Amyotrophic Lateral Sclerosis), McArdle Disease (Phosphorylase Deficiency), Merosin-Deficient Congenital Muscular Dystrophy, Metabolic Diseases of Muscle, Mitochondrial Myopathy, Miyoshi myopathy, Miyoshi Distal Myopathy, Motor Neurone Disease, Muscle-Eye-Brain Disease, Myasthenia Gravis (MG), Myoadenylate Deaminase Deficiency, Myofibrillar Myopathy, Myophosphorylase Deficiency, Myotonia Congenita (MC), Myotonic Muscular Dystrophy (MMD), Myotubular Myopathy (MTM or MM), Nemaline Myopathy, Nonaka Distal Myopathy, Oculopharyngeal Muscular Dystrophy (OPMD), Paramyotonia Congenita, Pearson Syndrome, Periodic Paralysis, Peroneal Muscular Atrophy (Charcot-Marie-Tooth Disease), Phosphofructokinase Deficiency, Phosphoglycerate Kinase Deficiency, Phosphoglycerate Mutase Deficiency, Phosphorylase Deficiency, Phosphorylase Deficiency, Polymyositis (PM), Pompe Disease (Acid Maltase Deficiency), Primary merosin deficiency (LAMA2), Progressive External Ophthalmoplegia (PEO), Rod Body Disease (Nemaline Myopathy), Spinal Muscular Atrophy (SMA), Spinal-Bulbar Muscular Atrophy (SBMA), Steinert Disease (Myotonic Muscular Dystrophy), Tarui Disease (Phosphofructokinase Deficiency), Thomsen Disease (Myotonia Congenita), Ullrich Congenital Muscular Dystrophy, Walker-Warburg Syndrome (Congenital Muscular Dystrophy), Welander Distal Myopathy, Werdnig-Hoffmann Disease (Spinal Muscular Atrophy), or ZASP-Related Myopathy.
   1227. The method of embodiment 1226, wherein the disease or condition of skeletal muscle is Acid Maltase Deficiency (AMD).
   1228. The method of embodiment 1226, wherein the disease or condition of skeletal muscle is Amyotrophic Lateral Sclerosis (ALS).
   1229. The method of embodiment 1226, wherein the disease or condition of skeletal muscle is Andersen-Tawil Syndrome.
   1230. The method of embodiment 1226, wherein the disease or condition of skeletal muscle is Barth syndrome (TAZ).
   1231. The method of embodiment 1226, wherein the disease or condition of skeletal muscle is Becker Muscular Dystrophy (BMD).
   1232. The method of embodiment 1226, wherein the disease or condition of skeletal muscle is Becker Myotonia Congenita.
   1233. The method of embodiment 1226, wherein the disease or condition of skeletal muscle is Bethlem Myopathy.
   1234. The method of embodiment 1226, wherein the disease or condition of skeletal muscle is Bulbospinal Muscular Atrophy (Spinal-Bulbar Muscular Atrophy).
   1235. The method of embodiment 1226, wherein the disease or condition of skeletal muscle is Carnitine Deficiency.
   1236. The method of embodiment 1226, wherein the disease or condition of skeletal muscle is Carnitine Palmityl Transferase Deficiency (CPT Deficiency).
   1237. The method of embodiment 1226, wherein the disease or condition of skeletal muscle is Central Core Disease (CCD).
   1238. The method of embodiment 1226, wherein the disease or condition of skeletal muscle is Centronuclear Myopathy.
   1239. The method of embodiment 1226, wherein the disease or condition of skeletal muscle is Charcot-Marie-Tooth Disease (CMT).
   1240. The method of embodiment 1226, wherein the disease or condition of skeletal muscle is Congenital Muscular Dystrophy (CMD).
   1241. The method of embodiment 1226, wherein the disease or condition of skeletal muscle is Congenital Myasthenic Syndromes (CMS).
   1242. The method of embodiment 1226, wherein the disease or condition of skeletal muscle is Congenital Myotonic Dystrophy.
   1243. The method of embodiment 1226, wherein the disease or condition of skeletal muscle is Cori Disease (Debrancher Enzyme Deficiency).
   1244. The method of embodiment 1226, wherein the disease or condition of skeletal muscle is Danon disease.
   1245. The method of embodiment 1226, wherein the disease or condition of skeletal muscle is Debrancher Enzyme Deficiency.
   1246. The method of embodiment 1226, wherein the disease or condition of skeletal muscle is Dejerine-Sottas Disease (DSD).
   1247. The method of embodiment 1226, wherein the disease or condition of skeletal muscle is Dermatomyositis (DM).
   1248. The method of embodiment 1226, wherein the disease or condition of skeletal muscle is Distal Muscular Dystrophy (DD).
   1249. The method of embodiment 1226, wherein the disease or condition of skeletal muscle is Distal myopathy with anterior tibial onset.
   1250. The method of embodiment 1226, wherein the disease or condition of skeletal muscle is Duchenne Muscular Dystrophy (DMD).
   1251. The method of embodiment 1226, wherein the disease or condition of skeletal muscle is Dystrophia Myotonica (Myotonic Muscular Dystrophy).
   1252. The method of embodiment 1226, wherein the disease or condition of skeletal muscle is Emery-Dreifuss Muscular Dystrophy (EDMD).
   1253. The method of embodiment 1224, wherein the disease or condition of skeletal muscle is an endocrine myopathy.
   1254. The method of embodiment 1226, wherein the disease or condition of skeletal muscle is Eulenberg Disease (Paramyotonia Congenita).
   1255. The method of embodiment 1226, wherein the disease or condition of skeletal muscle is Facioscapulohumeral Muscular Dystrophy (FSH or FSHD).
   1256. The method of embodiment 1226, wherein the disease or condition of skeletal muscle is Finnish (Tibial) Distal Myopathy.
   1257. The method of embodiment 1226, wherein the disease or condition of skeletal muscle is Forbes Disease (Debrancher Enzyme Deficiency).
   1258. The method of embodiment 1226, wherein the disease or condition of skeletal muscle is Friedreich's Ataxia (FA).
   1259. The method of embodiment 1226, wherein the disease or condition of skeletal muscle is Fukuyama Congenital Muscular Dystrophy.
   1260. The method of embodiment 1226, wherein the disease or condition of skeletal muscle is Glycogenosis Type 10.
   1261. The method of embodiment 1226, wherein the disease or condition of skeletal muscle is Glycogenosis Type 11.
   1262. The method of embodiment 1226, wherein the disease or condition of skeletal muscle is Glycogenosis Type 2.
   1263. The method of embodiment 1226, wherein the disease or condition of skeletal muscle is Glycogenosis Type 3.
   1264. The method of embodiment 1226, wherein the disease or condition of skeletal muscle is Glycogenosis Type 5.
   1265. The method of embodiment 1226, wherein the disease or condition of skeletal muscle is Glycogenosis Type 7.
   1266. The method of embodiment 1226, wherein the disease or condition of skeletal muscle is Glycogenosis Type 9.
   1267. The method of embodiment 1226, wherein the disease or condition of skeletal muscle is Gowers-Laing Distal Myopathy.
   1268. The method of embodiment 1226, wherein the disease or condition of skeletal muscle is Hauptmann-Thanheuser MD (Emery-Dreifuss Muscular Dystrophy).
   1269. The method of embodiment 1226, wherein the disease or condition of skeletal muscle is Hereditary Inclusion-Body Myositis.
   1270. The method of embodiment 1226, wherein the disease or condition of skeletal muscle is Hereditary Motor and Sensory Neuropathy (Charcot-Marie-Tooth Disease).
   1271. The method of embodiment 1226, wherein the disease or condition of skeletal muscle is Hyperthyroid Myopathy.
   1272. The method of embodiment 1226, wherein the disease or condition of skeletal muscle is Hypothyroid Myopathy.
   1273. The method of embodiment 1226, wherein the disease or condition of skeletal muscle is Inclusion-Body Myositis (IBM).
   1274. The method of embodiment 1226, wherein the disease or condition of skeletal muscle is an inherited myopathy.
   1275. The method of embodiment 1226, wherein the disease or condition of skeletal muscle is Integrin-Deficient Congenital Muscular Dystrophy.
   1276. The method of embodiment 1226, wherein the disease or condition of skeletal muscle is Kennedy Disease (Spinal-Bulbar Muscular Atrophy).
   1277. The method of embodiment 1226, wherein the disease or condition of skeletal muscle is Kugelberg-Welander Disease (Spinal Muscular Atrophy).
   1278. The method of embodiment 1226, wherein the disease or condition of skeletal muscle is Lactate Dehydrogenase Deficiency.
   1279. The method of embodiment 1226, wherein the disease or condition of skeletal muscle is Lambert-Eaton Myasthenic Syndrome (LEMS).
   1280. The method of embodiment 1226, wherein the disease or condition of skeletal muscle is Limb-Girdle Muscular Dystrophy (LGMD).
   1281. The method of embodiment 1226, wherein the disease or condition of skeletal muscle is Lou Gehrig's Disease (Amyotrophic Lateral Sclerosis).
   1282. The method of embodiment 1226, wherein the disease or condition of skeletal muscle is McArdle Disease (Phosphorylase Deficiency).
   1283. The method of embodiment 1226, wherein the disease or condition of skeletal muscle is Merosin-Deficient Congenital Muscular Dystrophy.
   1284. The method of embodiment 1226, wherein the disease or condition of skeletal muscle is Metabolic Diseases of Muscle.
   1285. The method of embodiment 1226, wherein the disease or condition of skeletal muscle is Mitochondrial Myopathy.
   1286. The method of embodiment 1226, wherein the disease or condition of skeletal muscle is Miyoshi myopathy.
   1287. The method of embodiment 1226, wherein the disease or condition of skeletal muscle is Miyoshi Distal Myopathy.
   1288. The method of embodiment 1226, wherein the disease or condition of skeletal muscle is Motor Neurone Disease.
   1289. The method of embodiment 1226, wherein the disease or condition of skeletal muscle is Muscle-Eye-Brain Disease.
   1290. The method of embodiment 1226, wherein the disease or condition of skeletal muscle is Myasthenia Gravis (MG).
   1291. The method of embodiment 1226, wherein the disease or condition of skeletal muscle is Myoadenylate Deaminase Deficiency.
   1292. The method of embodiment 1226, wherein the disease or condition of skeletal muscle is Myofibrillar Myopathy.
   1293. The method of embodiment 1226, wherein the disease or condition of skeletal muscle is Myophosphorylase Deficiency.
   1294. The method of embodiment 1226, wherein the disease or condition of skeletal muscle is Myotonia Congenita (MC).
   1295. The method of embodiment 1226, wherein the disease or condition of skeletal muscle is Myotonic Muscular Dystrophy (MMD).
   1296. The method of embodiment 1226, wherein the disease or condition of skeletal muscle is Myotubular Myopathy (MTM or MM).
   1297. The method of embodiment 1226, wherein the disease or condition of skeletal muscle is Nemaline Myopathy.
   1298. The method of embodiment 1226, wherein the disease or condition of skeletal muscle is Nonaka Distal Myopathy.
   1299. The method of embodiment 1226, wherein the disease or condition of skeletal muscle is Oculopharyngeal Muscular Dystrophy (OPMD).
   1300. The method of embodiment 1226, wherein the disease or condition of skeletal muscle is Paramyotonia Congenita.
   1301. The method of embodiment 1226, wherein the disease or condition of skeletal muscle is Pearson Syndrome.
   1302. The method of embodiment 1226, wherein the disease or condition of skeletal muscle is Periodic Paralysis.
   1303. The method of embodiment 1226, wherein the disease or condition of skeletal muscle is Peroneal Muscular Atrophy (Charcot-Marie-Tooth Disease).
   1304. The method of embodiment 1226, wherein the disease or condition of skeletal muscle is Phosphofructokinase Deficiency.
   1305. The method of embodiment 1226, wherein the disease or condition of skeletal muscle is Phosphoglycerate Kinase Deficiency.
   1306. The method of embodiment 1226, wherein the disease or condition of skeletal muscle is Phosphoglycerate Mutase Deficiency.
   1307. The method of embodiment 1226, wherein the disease or condition of skeletal muscle is Phosphorylase Deficiency.
   1308. The method of embodiment 1226, wherein the disease or condition of skeletal muscle is Polymyositis (PM).
   1309. The method of embodiment 1226, wherein the disease or condition of skeletal muscle is Pompe Disease (Acid Maltase Deficiency).
   1310. The method of embodiment 1226, wherein the disease or condition of skeletal muscle is Primary merosin deficiency (LAMA2).
   1311. The method of embodiment 1226, wherein the disease or condition of skeletal muscle is Progressive External Ophthalmoplegia (PEO).
   1312. The method of embodiment 1226, wherein the disease or condition of skeletal muscle is Rod Body Disease (Nemaline Myopathy).
   1313. The method of embodiment 1226, wherein the disease or condition of skeletal muscle is Muscular Atrophy (SMA).
   1314. The method of embodiment 1226, wherein the disease or condition of skeletal muscle is Spinal-Bulbar Muscular Atrophy (SBMA).
   1315. The method of embodiment 1226, wherein the disease or condition of skeletal muscle is Steinert Disease (Myotonic Muscular Dystrophy).
   1316. The method of embodiment 1226, wherein the disease or condition of skeletal muscle is Tarui Disease (Phosphofructokinase Deficiency).
   1317. The method of embodiment 1226, wherein the disease or condition of skeletal muscle is Thomsen Disease (Myotonia Congenita).
   1318. The method of embodiment 1226, wherein the disease or condition of skeletal muscle is Ullrich Congenital Muscular Dystrophy.
   1319. The method of embodiment 1226, wherein the disease or condition of skeletal muscle is Walker-Warburg Syndrome (Congenital Muscular Dystrophy).
   1320. The method of embodiment 1226, wherein the disease or condition of skeletal muscle is Welander Distal Myopathy.
   1321. The method of embodiment 1226, wherein the disease or condition of skeletal muscle is Werdnig-Hoffmann Disease (Spinal Muscular Atrophy).
   1322. The method of embodiment 1226, wherein the disease or condition of skeletal muscle is ZASP-Related Myopathy.
   1323. The method of embodiment 748, wherein the disease or condition is Achromatopsia (color blindness) and/or wherein the heterologous nucleic acid sequence encodes Cyclic nucleotide-gated cation channel alpha-3 (CNGA3) (e.g., a polypeptide represented by UniProt Accession number Q16281 or a polypeptide comprising an amino acid sequence having at least 95% sequence identity thereto).
   1324. The method of embodiment 748, wherein the disease or condition is Achromatopsia (color blindness) and/or wherein the heterologous nucleic acid sequence encodes Cyclic nucleotide-gated cation channel beta-3 (CNGB3) (e.g., a polypeptide represented by UniProt Accession number Q9NQW8 or a polypeptide comprising an amino acid sequence having at least 95% sequence identity thereto).
   1325. The method of embodiment 748, wherein the disease or condition is Achromatopsia (color blindness) and/or wherein the heterologous nucleic acid sequence encodes Guanine nucleotide-binding protein G(t) subunit alpha-2 (GNAT2) (e.g., a polypeptide represented by UniProt Accession number P19087 or a polypeptide comprising an amino acid sequence having at least 95% sequence identity thereto).
   1326. The method of embodiment 748, wherein the disease or condition is Achromatopsia (color blindness) and/or wherein the heterologous nucleic acid sequence encodes Cone cGMP-specific 3′,5′-cyclic phosphodiesterase subunit alpha (PDE6C) (e.g., a polypeptide represented by UniProt Accession number P51160 or a polypeptide comprising an amino acid sequence having at least 95% sequence identity thereto).
   1327. The method of embodiment 748, wherein the disease or condition is Acute Intermittent Porphyria and/or wherein the heterologous nucleic acid sequence encodes Porphobilinogen deaminase (PBGD), HMBS (e.g., a polypeptide represented by UniProt Accession number P08397 or a polypeptide comprising an amino acid sequence having at least 95% sequence identity thereto).
   1328. The method of embodiment 748, wherein the disease or condition is Adie syndrome (Adie's pupil) and/or wherein the heterologous nucleic acid sequence encodes MPZ (e.g., a polypeptide represented by UniProt Accession number P25189 or a polypeptide comprising an amino acid sequence having at least 95% sequence identity thereto).
   1329. The method of embodiment 748, wherein the disease or condition is Age-Related Macular Degeneration and/or wherein the heterologous nucleic acid sequence encodes Vascular endothelial growth factor receptor 1 (FLT1) (e.g., a polypeptide represented by UniProt Accession number P17948 or a polypeptide comprising an amino acid sequence having at least 95% sequence identity thereto).
   1330. The method of embodiment 748, wherein the disease or condition is Age-Related Macular Degeneration and/or wherein the heterologous nucleic acid sequence encodes Vascular endothelial growth factor A (VEGFA) (e.g., a polypeptide represented by UniProt Accession number P15692 or a polypeptide comprising an amino acid sequence having at least 95% sequence identity thereto).
   1331. The method of embodiment 748, wherein the disease or condition is Agenesis of the Corpus Callosum (ACCPN) and/or wherein the heterologous nucleic acid sequence encodes SLC12A6 (e.g., a polypeptide represented by UniProt Accession number Q9UHW9 or a polypeptide comprising an amino acid sequence having at least 95% sequence identity thereto).
   1332. The method of embodiment 748, wherein the disease or condition is Aicardi-Goutieres syndrome and/or wherein the heterologous nucleic acid sequence encodes TREX1 (e.g., a polypeptide represented by UniProt Accession number Q9NSU2 or a polypeptide comprising an amino acid sequence having at least 95% sequence identity thereto).
   1333. The method of embodiment 748, wherein the disease or condition is Alexander disease and/or wherein the heterologous nucleic acid sequence encodes GFAP (e.g., a polypeptide represented by UniProt Accession number P14136 or a polypeptide comprising an amino acid sequence having at least 95% sequence identity thereto).
   1334. The method of embodiment 748, wherein the disease or condition is Alpers syndrome and/or wherein the heterologous nucleic acid sequence encodes POLG (e.g., a polypeptide represented by UniProt Accession number P54098 or a polypeptide comprising an amino acid sequence having at least 95% sequence identity thereto).
   1335. The method of embodiment 748, wherein the disease or condition is Alternating hemiplegia and/or wherein the heterologous nucleic acid sequence encodes ATP1A2 (e.g., a polypeptide represented by UniProt Accession number P50993 or a polypeptide comprising an amino acid sequence having at least 95% sequence identity thereto).
   1336. The method of embodiment 748, wherein the disease or condition is Alternating hemiplegia and/or wherein the heterologous nucleic acid sequence encodes ATP1A3 (e.g., a polypeptide represented by UniProt Accession number P13637 or a polypeptide comprising an amino acid sequence having at least 95% sequence identity thereto).
   1337. The method of embodiment 748, wherein the disease or condition is Alzheimer's disease and/or wherein the heterologous nucleic acid sequence encodes NGF (e.g., a polypeptide represented by UniProt Accession number P01138 or a polypeptide comprising an amino acid sequence having at least 95% sequence identity thereto).
   1338. The method of embodiment 748, wherein the disease or condition is Alzheimer's disease and/or wherein the heterologous nucleic acid sequence encodes ApoE (e.g., a polypeptide represented by UniProt Accession number P02649 or a polypeptide comprising an amino acid sequence having at least 95% sequence identity thereto).
   1339. The method of embodiment 748, wherein the disease or condition is Alzheimer's disease and/or wherein the heterologous nucleic acid sequence encodes Presenilin (PSEN1) (e.g., a polypeptide represented by UniProt Accession number A0A024R6A3 or a polypeptide comprising an amino acid sequence having at least 95% sequence identity thereto).
   1340. The method of embodiment 748, wherein the disease or condition is Alzheimer's disease and/or wherein the heterologous nucleic acid sequence encodes Presenilin-2 (PSEN2) (e.g., a polypeptide represented by UniProt Accession number P49810 or a polypeptide comprising an amino acid sequence having at least 95% sequence identity thereto).
   1341. The method of embodiment 748, wherein the disease or condition is Alzheimer's disease and/or wherein the heterologous nucleic acid sequence encodes Amyloid-beta precursor protein (APP) (e.g., a polypeptide represented by UniProt Accession number P05067 or a polypeptide comprising an amino acid sequence having at least 95% sequence identity thereto).
   1342. The method of embodiment 748, wherein the disease or condition is Alzheimer's disease and/or wherein the heterologous nucleic acid sequence encodes ADAM10 (e.g., a polypeptide represented by UniProt Accession number 014672 or a polypeptide comprising an amino acid sequence having at least 95% sequence identity thereto).
   1343. The method of embodiment 748, wherein the disease or condition is Alzheimer's disease and/or wherein the heterologous nucleic acid sequence encodes MAPT, Tau (e.g., a polypeptide represented by UniProt Accession number P10636 or a polypeptide comprising an amino acid sequence having at least 95% sequence identity thereto).
   1344. The method of embodiment 748, wherein the disease or condition is Amyotrophic lateral sclerosis (ALS) (Lou Gehrig's disease) and/or wherein the heterologous nucleic acid sequence encodes Superoxide dismutase-1 (SOD1) (e.g., a polypeptide represented by UniProt Accession number P00441 or a polypeptide comprising an amino acid sequence having at least 95% sequence identity thereto).
   1345. The method of embodiment 748, wherein the disease or condition is Amyotrophy, hereditary neuralgic and/or wherein the heterologous nucleic acid sequence encodes 45544 (e.g., a polypeptide represented by UniProt Accession number Q9UHD8 or a polypeptide comprising an amino acid sequence having at least 95% sequence identity thereto).
   1346. The method of embodiment 748, wherein the disease or condition is Angleman syndrome and/or wherein the heterologous nucleic acid sequence encodes Ubiquitin-protein ligase E3A (UBE3A) (e.g., a polypeptide represented by UniProt Accession number Q05086 or a polypeptide comprising an amino acid sequence having at least 95% sequence identity thereto).
   1347. The method of embodiment 748, wherein the disease or condition is Aromatic L-amino acid decarboxylase deficiency (AADCD) and/or wherein the heterologous nucleic acid sequence encodes DDC (e.g., a polypeptide represented by UniProt Accession number P20711 or a polypeptide comprising an amino acid sequence having at least 95% sequence identity thereto).
   1348. The method of embodiment 748, wherein the disease or condition is Ataxia and/or wherein the heterologous nucleic acid sequence encodes APTX (e.g., a polypeptide represented by UniProt Accession number Q7Z2E3 or a polypeptide comprising an amino acid sequence having at least 95% sequence identity thereto).
   1349. The method of embodiment 748, wherein the disease or condition is Ataxia and/or wherein the heterologous nucleic acid sequence encodes KCNA1 (e.g., a polypeptide represented by UniProt Accession number Q09470 or a polypeptide comprising an amino acid sequence having at least 95% sequence identity thereto).
   1350. The method of embodiment 748, wherein the disease or condition is Ataxia and/or wherein the heterologous nucleic acid sequence encodes CACNA1A (e.g., a polypeptide represented by UniProt Accession number 000555 or a polypeptide comprising an amino acid sequence having at least 95% sequence identity thereto).
   1351. The method of embodiment 748, wherein the disease or condition is Ataxia Telangiectasia (Louis-Bar syndrome) and/or wherein the heterologous nucleic acid sequence encodes Serine-protein kinase ATM (ATM) (e.g., a polypeptide represented by UniProt Accession number Q13315 or a polypeptide comprising an amino acid sequence having at least 95% sequence identity thereto).
   1352. The method of embodiment 748, wherein the disease or condition is Attention deficit hyperactivity disorder (ADHD) and/or wherein the heterologous nucleic acid sequence encodes DRD4 (e.g., a polypeptide represented by UniProt Accession number P21917 or a polypeptide comprising an amino acid sequence having at least 95% sequence identity thereto).
   1353. The method of embodiment 748, wherein the disease or condition is Attention deficit hyperactivity disorder (ADHD) and/or wherein the heterologous nucleic acid sequence encodes CDH2 (e.g., a polypeptide represented by UniProt Accession number P19022 or a polypeptide comprising an amino acid sequence having at least 95% sequence identity thereto).
   1354. The method of embodiment 748, wherein the disease or condition is Becker muscular dystrophy and/or wherein the heterologous nucleic acid sequence encodes Follistatin (FST) (e.g., a polypeptide represented by UniProt Accession number P19883 or a polypeptide comprising an amino acid sequence having at least 95% sequence identity thereto).
   1355. The method of embodiment 748, wherein the disease or condition is Becker muscular dystrophy and/or wherein the heterologous nucleic acid sequence encodes DMD (e.g., a polypeptide represented by UniProt Accession number P11532 or a polypeptide comprising an amino acid sequence having at least 95% sequence identity thereto).
   1356. The method of embodiment 748, wherein the disease or condition is Benign essential blepharospasm and/or wherein the heterologous nucleic acid sequence encodes DRD5 (e.g., a polypeptide represented by UniProt Accession number P21918 or a polypeptide comprising an amino acid sequence having at least 95% sequence identity thereto).
   1357. The method of embodiment 748, wherein the disease or condition is Bradbury-Eggleston syndrome (pure autonomic failure) and/or wherein the heterologous nucleic acid sequence encodes COQ2 (e.g., a polypeptide represented by UniProt Accession number Q96H96 or a polypeptide comprising an amino acid sequence having at least 95% sequence identity thereto).
   1358. The method of embodiment 748, wherein the disease or condition is Bulbar palsy (BVVLS1) and/or wherein the heterologous nucleic acid sequence encodes SLC52A3 (e.g., a polypeptide represented by UniProt Accession number Q9NQ40 or a polypeptide comprising an amino acid sequence having at least 95% sequence identity thereto).
   1359. The method of embodiment 748, wherein the disease or condition is Canavan disease (aminoacylase 2 deficiency) and/or wherein the heterologous nucleic acid sequence encodes ASPA (e.g., a polypeptide represented by UniProt Accession number P45381 or a polypeptide comprising an amino acid sequence having at least 95% sequence identity thereto).
   1360. The method of embodiment 748, wherein the disease or condition is Carpal tunnel syndrome and/or wherein the heterologous nucleic acid sequence encodes TTR (e.g., a polypeptide represented by UniProt Accession number P02766 or a polypeptide comprising an amino acid sequence having at least 95% sequence identity thereto).
   1361. The method of embodiment 748, wherein the disease or condition is Cavernoma (Cavernous angioma, Cavernous malformations) and/or wherein the heterologous nucleic acid sequence encodes KRIT1 (e.g., a polypeptide represented by UniProt Accession number 000522 or a polypeptide comprising an amino acid sequence having at least 95% sequence identity thereto).
   1362. The method of embodiment 748, wherein the disease or condition is Cerebellar Hypoplasia (CHEGDD) and/or wherein the heterologous nucleic acid sequence encodes OXR1 (e.g., a polypeptide represented by UniProt Accession number Q8N573 or a polypeptide comprising an amino acid sequence having at least 95% sequence identity thereto).
   1363. The method of embodiment 748, wherein the disease or condition is Cerebellar ataxia (CAMRQ2) and/or wherein the heterologous nucleic acid sequence encodes WDR81 (e.g., a polypeptide represented by UniProt Accession number Q562E7 or a polypeptide comprising an amino acid sequence having at least 95% sequence identity thereto).
   1364. The method of embodiment 748, wherein the disease or condition is Cerebral Arteriopathy with SCI and Leukoencephalopathy (CADASIL) and/or wherein the heterologous nucleic acid sequence encodes NOTCH3 (e.g., a polypeptide represented by UniProt Accession number Q9UM47 or a polypeptide comprising an amino acid sequence having at least 95% sequence identity thereto).
   1365. The method of embodiment 748, wherein the disease or condition is Cerebral gigantism (Sotos syndrome 1) and/or wherein the heterologous nucleic acid sequence encodes NSD1 (e.g., a polypeptide represented by UniProt Accession number Q96L73 or a polypeptide comprising an amino acid sequence having at least 95% sequence identity thereto).
   1366. The method of embodiment 748, wherein the disease or condition is Cerebro-oculo-facio-skeletal syndrome (COFS) and/or wherein the heterologous nucleic acid sequence encodes ERCC6 (e.g., a polypeptide represented by UniProt Accession number Q03468 or a polypeptide comprising an amino acid sequence having at least 95% sequence identity thereto).
   1367. The method of embodiment 748, wherein the disease or condition is Ceroid lipofuscinosis (Batten disease) and/or wherein the heterologous nucleic acid sequence encodes CLN1 (PPT1) (e.g., a polypeptide represented by UniProt Accession number P50897 or a polypeptide comprising an amino acid sequence having at least 95% sequence identity thereto).
   1368. The method of embodiment 748, wherein the disease or condition is Ceroid lipofuscinosis (Batten disease) and/or wherein the heterologous nucleic acid sequence encodes CLN2 (TPP1) (e.g., a polypeptide represented by UniProt Accession number 014773 or a polypeptide comprising an amino acid sequence having at least 95% sequence identity thereto).
   1369. The method of embodiment 748, wherein the disease or condition is Ceroid lipofuscinosis (Batten disease) and/or wherein the heterologous nucleic acid sequence encodes CLN3 (battenin) (e.g., a polypeptide represented by UniProt Accession number Q13286 or a polypeptide comprising an amino acid sequence having at least 95% sequence identity thereto).
   1370. The method of embodiment 748, wherein the disease or condition is Ceroid lipofuscinosis (Batten disease) and/or wherein the heterologous nucleic acid sequence encodes CLN4 (e.g., a polypeptide represented by UniProt Accession number Q9H3Z4 or a polypeptide comprising an amino acid sequence having at least 95% sequence identity thereto).
   1371. The method of embodiment 748, wherein the disease or condition is Ceroid lipofuscinosis (Batten disease) and/or wherein the heterologous nucleic acid sequence encodes CLN5 (e.g., a polypeptide represented by UniProt Accession number 075503 or a polypeptide comprising an amino acid sequence having at least 95% sequence identity thereto).
   1372. The method of embodiment 748, wherein the disease or condition is Ceroid lipofuscinosis (Batten disease) and/or wherein the heterologous nucleic acid sequence encodes CLN6 (e.g., a polypeptide represented by UniProt Accession number Q9NVWV5 or a polypeptide comprising an amino acid sequence having at least 95% sequence identity thereto).
   1373. The method of embodiment 748, wherein the disease or condition is Ceroid lipofuscinosis (Batten disease) and/or wherein the heterologous nucleic acid sequence encodes CLN7 (MFSD8) (e.g., a polypeptide represented by UniProt Accession number Q8NHS3 or a polypeptide comprising an amino acid sequence having at least 95% sequence identity thereto).
   1374. The method of embodiment 748, wherein the disease or condition is Ceroid lipofuscinosis (Batten disease) and/or wherein the heterologous nucleic acid sequence encodes CLN8 (e.g., a polypeptide represented by UniProt Accession number Q9UBY8 or a polypeptide comprising an amino acid sequence having at least 95% sequence identity thereto).
   1375. The method of embodiment 748, wherein the disease or condition is Ceroid lipofuscinosis (Batten disease) and/or wherein the heterologous nucleic acid sequence encodes CLN10 (cathepsin D) (e.g., a polypeptide represented by UniProt Accession number P07339 or a polypeptide comprising an amino acid sequence having at least 95% sequence identity thereto).
   1376. The method of embodiment 748, wherein the disease or condition is Ceroid lipofuscinosis (Batten disease) and/or wherein the heterologous nucleic acid sequence encodes CLN11 (progranulin) (e.g., a polypeptide represented by UniProt Accession number P28799 or a polypeptide comprising an amino acid sequence having at least 95% sequence identity thereto).
   1377. The method of embodiment 748, wherein the disease or condition is Ceroid lipofuscinosis (Batten disease) and/or wherein the heterologous nucleic acid sequence encodes CLN12 (ATP13A2) (e.g., a polypeptide represented by UniProt Accession number Q9NQ11 or a polypeptide comprising an amino acid sequence having at least 95% sequence identity thereto).
   1378. The method of embodiment 748, wherein the disease or condition is Ceroid lipofuscinosis (Batten disease) and/or wherein the heterologous nucleic acid sequence encodes CLN13 (cathepsin F) (e.g., a polypeptide represented by UniProt Accession number Q9UBX1 or a polypeptide comprising an amino acid sequence having at least 95% sequence identity thereto).
   1379. The method of embodiment 748, wherein the disease or condition is Ceroid lipofuscinosis (Batten disease) and/or wherein the heterologous nucleic acid sequence encodes CLN14 (KCTD7) (e.g., a polypeptide represented by UniProt Accession number Q96MP8 or a polypeptide comprising an amino acid sequence having at least 95% sequence identity thereto).
   1380. The method of embodiment 748, wherein the disease or condition is Charcot-Marie-Tooth disease and/or wherein the heterologous nucleic acid sequence encodes PMP22 (e.g., a polypeptide represented by UniProt Accession number Q01453 or a polypeptide comprising an amino acid sequence having at least 95% sequence identity thereto).
   1381. The method of embodiment 748, wherein the disease or condition is Charcot-Marie-Tooth disease and/or wherein the heterologous nucleic acid sequence encodes MPZ (e.g., a polypeptide represented by UniProt Accession number P25189 or a polypeptide comprising an amino acid sequence having at least 95% sequence identity thereto).
   1382. The method of embodiment 748, wherein the disease or condition is Charcot-Marie-Tooth disease and/or wherein the heterologous nucleic acid sequence encodes DNM2 (e.g., a polypeptide represented by UniProt Accession number P50570 or a polypeptide comprising an amino acid sequence having at least 95% sequence identity thereto).
   1383. The method of embodiment 748, wherein the disease or condition is Charcot-Marie-Tooth disease and/or wherein the heterologous nucleic acid sequence encodes MFN2 (e.g., a polypeptide represented by UniProt Accession number 095140 or a polypeptide comprising an amino acid sequence having at least 95% sequence identity thereto).
   1384. The method of embodiment 748, wherein the disease or condition is Charcot-Marie-Tooth disease and/or wherein the heterologous nucleic acid sequence encodes KIF1B (e.g., a polypeptide represented by UniProt Accession number 060333 or a polypeptide comprising an amino acid sequence having at least 95% sequence identity thereto).
   1385. The method of embodiment 748, wherein the disease or condition is Charcot-Marie-Tooth disease and/or wherein the heterologous nucleic acid sequence encodes SBF2 (e.g., a polypeptide represented by UniProt Accession number Q86WG5 or a polypeptide comprising an amino acid sequence having at least 95% sequence identity thereto).
   1386. The method of embodiment 748, wherein the disease or condition is Charcot-Marie-Tooth disease and/or wherein the heterologous nucleic acid sequence encodes PNKP (e.g., a polypeptide represented by UniProt Accession number Q96T60 or a polypeptide comprising an amino acid sequence having at least 95% sequence identity thereto).
   1387. The method of embodiment 748, wherein the disease or condition is Charcot-Marie-Tooth disease and/or wherein the heterologous nucleic acid sequence encodes GDAP1 (e.g., a polypeptide represented by UniProt Accession number Q8TB36 or a polypeptide comprising an amino acid sequence having at least 95% sequence identity thereto).
   1388. The method of embodiment 748, wherein the disease or condition is Charcot-Marie-Tooth disease and/or wherein the heterologous nucleic acid sequence encodes LMNA (e.g., a polypeptide represented by UniProt Accession number P02545 or a polypeptide comprising an amino acid sequence having at least 95% sequence identity thereto).
   1389. The method of embodiment 748, wherein the disease or condition is Charcot-Marie-Tooth disease and/or wherein the heterologous nucleic acid sequence encodes FGD4 (e.g., a polypeptide represented by UniProt Accession number Q96M96 or a polypeptide comprising an amino acid sequence having at least 95% sequence identity thereto).
   1390. The method of embodiment 748, wherein the disease or condition is Charcot-Marie-Tooth disease and/or wherein the heterologous nucleic acid sequence encodes MTMR2 (e.g., a polypeptide represented by UniProt Accession number Q13614 or a polypeptide comprising an amino acid sequence having at least 95% sequence identity thereto).
   1391. The method of embodiment 748, wherein the disease or condition is Chorea and/or wherein the heterologous nucleic acid sequence encodes NKX2-1 (e.g., a polypeptide represented by UniProt Accession number P43699 or a polypeptide comprising an amino acid sequence having at least 95% sequence identity thereto).
   1392. The method of embodiment 748, wherein the disease or condition is Choreoacanthocytosis and/or wherein the heterologous nucleic acid sequence encodes VPS13A (e.g., a polypeptide represented by UniProt Accession number Q96RL7 or a polypeptide comprising an amino acid sequence having at least 95% sequence identity thereto).
   1393. The method of embodiment 748, wherein the disease or condition is Choroideremia and/or wherein the heterologous nucleic acid sequence encodes Rab escort protein (Rep1), CHM (e.g., a polypeptide represented by UniProt Accession number P24386 or a polypeptide comprising an amino acid sequence having at least 95% sequence identity thereto).
   1394. The method of embodiment 748, wherein the disease or condition is Cockayne syndrome B (CSB) and/or wherein the heterologous nucleic acid sequence encodes ERCC6 (e.g., a polypeptide represented by UniProt Accession number Q03468 or a polypeptide comprising an amino acid sequence having at least 95% sequence identity thereto).
   1395. The method of embodiment 748, wherein the disease or condition is Coffin-Lowry syndrome and/or wherein the heterologous nucleic acid sequence encodes RPS6KA3 (e.g., a polypeptide represented by UniProt Accession number P51812 or a polypeptide comprising an amino acid sequence having at least 95% sequence identity thereto).
   1396. The method of embodiment 748, wherein the disease or condition is Craniosynostosis and/or wherein the heterologous nucleic acid sequence encodes MSX2 (e.g., a polypeptide represented by UniProt Accession number P35548 or a polypeptide comprising an amino acid sequence having at least 95% sequence identity thereto).
   1397. The method of embodiment 748, wherein the disease or condition is Craniosynostosis and/or wherein the heterologous nucleic acid sequence encodes TWIST1 (e.g., a polypeptide represented by UniProt Accession number Q15672 or a polypeptide comprising an amino acid sequence having at least 95% sequence identity thereto).
   1398. The method of embodiment 748, wherein the disease or condition is Craniosynostosis and/or wherein the heterologous nucleic acid sequence encodes SKI (e.g., a polypeptide represented by UniProt Accession number P12755 or a polypeptide comprising an amino acid sequence having at least 95% sequence identity thereto).
   1399. The method of embodiment 748, wherein the disease or condition is Craniosynostosis and/or wherein the heterologous nucleic acid sequence encodes SMAD6 (e.g., a polypeptide represented by UniProt Accession number 043541 or a polypeptide comprising an amino acid sequence having at least 95% sequence identity thereto).
   1400. The method of embodiment 748, wherein the disease or condition is Creutzfeldt-Jakob disease and/or wherein the heterologous nucleic acid sequence encodes PRNP (e.g., a polypeptide represented by UniProt Accession number P04156 or a polypeptide comprising an amino acid sequence having at least 95% sequence identity thereto).
   1401. The method of embodiment 748, wherein the disease or condition is Creutzfeldt-Jakob disease and/or wherein the heterologous nucleic acid sequence encodes HLA-DQB1 (e.g., a polypeptide represented by UniProt Accession number P01920 or a polypeptide comprising an amino acid sequence having at least 95% sequence identity thereto).
   1402. The method of embodiment 748, wherein the disease or condition is Crigler-Najjar Syndrome (hyperbilirubinemia) and/or wherein the heterologous nucleic acid sequence encodes UDP-glucuronosyltransferase 1A1 (UGT1A1) (e.g., a polypeptide represented by UniProt Accession number P22309 or a polypeptide comprising an amino acid sequence having at least 95% sequence identity thereto).
   1403. The method of embodiment 748, wherein the disease or condition is Cushing Syndrome and/or wherein the heterologous nucleic acid sequence encodes PRKACA (e.g., a polypeptide represented by UniProt Accession number P17612 or a polypeptide comprising an amino acid sequence having at least 95% sequence identity thereto).
   1404. The method of embodiment 748, wherein the disease or condition is Dentatorubral atrophy (DRPLA) and/or wherein the heterologous nucleic acid sequence encodes ATN1 (e.g., a polypeptide represented by UniProt Accession number P54259 or a polypeptide comprising an amino acid sequence having at least 95% sequence identity thereto).
   1405. The method of embodiment 748, wherein the disease or condition is Developmental and Epileptic Encephalopathy and/or wherein the heterologous nucleic acid sequence encodes ARX (e.g., a polypeptide represented by UniProt Accession number Q96QS3 or a polypeptide comprising an amino acid sequence having at least 95% sequence identity thereto).
   1406. The method of embodiment 748, wherein the disease or condition is Developmental and Epileptic Encephalopathy and/or wherein the heterologous nucleic acid sequence encodes FGF12 (e.g., a polypeptide represented by UniProt Accession number P61328 or a polypeptide comprising an amino acid sequence having at least 95% sequence identity thereto).
   1407. The method of embodiment 748, wherein the disease or condition is Developmental and Epileptic Encephalopathy and/or wherein the heterologous nucleic acid sequence encodes PIGP (e.g., a polypeptide represented by UniProt Accession number P57054 or a polypeptide comprising an amino acid sequence having at least 95% sequence identity thereto).
   1408. The method of embodiment 748, wherein the disease or condition is Developmental and Epileptic Encephalopathy and/or wherein the heterologous nucleic acid sequence encodes GABRB3 (e.g., a polypeptide represented by UniProt Accession number P28472 or a polypeptide comprising an amino acid sequence having at least 95% sequence identity thereto).
   1409. The method of embodiment 748, wherein the disease or condition is Developmental and Epileptic Encephalopathy and/or wherein the heterologous nucleic acid sequence encodes NECAP1 (e.g., a polypeptide represented by UniProt Accession number Q8NC96 or a polypeptide comprising an amino acid sequence having at least 95% sequence identity thereto).
   1410. The method of embodiment 748, wherein the disease or condition is Developmental Dyspraxia (speech-language disorder 1 (SPCH1)) and/or wherein the heterologous nucleic acid sequence encodes FOXP2 (e.g., a polypeptide represented by UniProt Accession number 015409 or a polypeptide comprising an amino acid sequence having at least 95% sequence identity thereto).
   1411. The method of embodiment 748, wherein the disease or condition is Dravet syndrome and/or wherein the heterologous nucleic acid sequence encodes Sodium channel protein type 1 subunit alpha (SCN1A) (e.g., a polypeptide represented by UniProt Accession number P35498 or a polypeptide comprising an amino acid sequence having at least 95% sequence identity thereto).
   1412. The method of embodiment 748, wherein the disease or condition is Dravet syndrome and/or wherein the heterologous nucleic acid sequence encodes SCN1B (e.g., a polypeptide represented by UniProt Accession number Q07699 or a polypeptide comprising an amino acid sequence having at least 95% sequence identity thereto).
   1413. The method of embodiment 748, wherein the disease or condition is Dravet syndrome and/or wherein the heterologous nucleic acid sequence encodes SCN2A (e.g., a polypeptide represented by UniProt Accession number Q99250 or a polypeptide comprising an amino acid sequence having at least 95% sequence identity thereto).
   1414. The method of embodiment 748, wherein the disease or condition is Dravet syndrome and/or wherein the heterologous nucleic acid sequence encodes GABA receptor subunit gamma-2 (GABRG2) (e.g., a polypeptide represented by UniProt Accession number P18507 or a polypeptide comprising an amino acid sequence having at least 95% sequence identity thereto).
   1415. The method of embodiment 748, wherein the disease or condition is Dysautonomia (Day syndrome) and/or wherein the heterologous nucleic acid sequence encodes ELP1 (e.g., a polypeptide represented by UniProt Accession number 095163 or a polypeptide comprising an amino acid sequence having at least 95% sequence identity thereto).
   1416. The method of embodiment 748, wherein the disease or condition is Dystonia and/or wherein the heterologous nucleic acid sequence encodes GCH1 (e.g., a polypeptide represented by UniProt Accession number P30793 or a polypeptide comprising an amino acid sequence having at least 95% sequence identity thereto).
   1417. The method of embodiment 748, wherein the disease or condition is Dystonia and/or wherein the heterologous nucleic acid sequence encodes TOR1A (e.g., a polypeptide represented by UniProt Accession number 014656 or a polypeptide comprising an amino acid sequence having at least 95% sequence identity thereto).
   1418. The method of embodiment 748, wherein the disease or condition is Dystonia and/or wherein the heterologous nucleic acid sequence encodes SGCE (e.g., a polypeptide represented by UniProt Accession number 043556 or a polypeptide comprising an amino acid sequence having at least 95% sequence identity thereto).
   1419. The method of embodiment 748, wherein the disease or condition is Dystonia and/or wherein the heterologous nucleic acid sequence encodes TUBB4A (e.g., a polypeptide represented by UniProt Accession number P04350 or a polypeptide comprising an amino acid sequence having at least 95% sequence identity thereto).
   1420. The method of embodiment 748, wherein the disease or condition is Encephalocele and/or wherein the heterologous nucleic acid sequence encodes COL18A1 (e.g., a polypeptide represented by UniProt Accession number P39060 or a polypeptide comprising an amino acid sequence having at least 95% sequence identity thereto).
   1421. The method of embodiment 748, wherein the disease or condition is Epilepsy disorder and/or wherein the heterologous nucleic acid sequence encodes GRIN2A (e.g., a polypeptide represented by UniProt Accession number Q12879 or a polypeptide comprising an amino acid sequence having at least 95% sequence identity thereto).
   1422. The method of embodiment 748, wherein the disease or condition is Epilepsy disorder and/or wherein the heterologous nucleic acid sequence encodes CSTB (e.g., a polypeptide represented by UniProt Accession number P04080 or a polypeptide comprising an amino acid sequence having at least 95% sequence identity thereto).
   1423. The method of embodiment 748, wherein the disease or condition is Epilepsy disorder and/or wherein the heterologous nucleic acid sequence encodes STARD7 (e.g., a polypeptide represented by UniProt Accession number Q9NQZ5 or a polypeptide comprising an amino acid sequence having at least 95% sequence identity thereto).
   1424. The method of embodiment 748, wherein the disease or condition is Epilepsy disorder and/or wherein the heterologous nucleic acid sequence encodes DEPDC5 (e.g., a polypeptide represented by UniProt Accession number 075140 or a polypeptide comprising an amino acid sequence having at least 95% sequence identity thereto).
   1425. The method of embodiment 748, wherein the disease or condition is Epilepsy disorder and/or wherein the heterologous nucleic acid sequence encodes PCDH19 (e.g., a polypeptide represented by UniProt Accession number Q8TAB3 or a polypeptide comprising an amino acid sequence having at least 95% sequence identity thereto).
   1426. The method of embodiment 748, wherein the disease or condition is Essential tremor and/or wherein the heterologous nucleic acid sequence encodes DRD3 (e.g., a polypeptide represented by UniProt Accession number P35462 or a polypeptide comprising an amino acid sequence having at least 95% sequence identity thereto).
   1427. The method of embodiment 748, wherein the disease or condition is Essential tremor and/or wherein the heterologous nucleic acid sequence encodes NOTCH2NLC (e.g., a polypeptide represented by UniProt Accession number PODPK4 or a polypeptide comprising an amino acid sequence having at least 95% sequence identity thereto).
   1428. The method of embodiment 748, wherein the disease or condition is Essential tremor and/or wherein the heterologous nucleic acid sequence encodes FUS (e.g., a polypeptide represented by UniProt Accession number P35637 or a polypeptide comprising an amino acid sequence having at least 95% sequence identity thereto).
   1429. The method of embodiment 748, wherein the disease or condition is Fabry disease and/or wherein the heterologous nucleic acid sequence encodes alpha-galactosidase A (GLA) (e.g., a polypeptide represented by UniProt Accession number P06280 or a polypeptide comprising an amino acid sequence having at least 95% sequence identity thereto).
   1430. The method of embodiment 748, wherein the disease or condition is Farber disease (ceramidase deficiency) and/or wherein the heterologous nucleic acid sequence encodes ASAH1 (e.g., a polypeptide represented by UniProt Accession number Q13510 or a polypeptide comprising an amino acid sequence having at least 95% sequence identity thereto).
   1431. The method of embodiment 748, wherein the disease or condition is Fahr disease and/or wherein the heterologous nucleic acid sequence encodes SLC20A2 (e.g., a polypeptide represented by UniProt Accession number Q08357 or a polypeptide comprising an amino acid sequence having at least 95% sequence identity thereto).
   1432. The method of embodiment 748, wherein the disease or condition is Febrile Seizure and/or wherein the heterologous nucleic acid sequence encodes GABRG2 (e.g., a polypeptide represented by UniProt Accession number P18507 or a polypeptide comprising an amino acid sequence having at least 95% sequence identity thereto).
   1433. The method of embodiment 748, wherein the disease or condition is Febrile Seizure and/or wherein the heterologous nucleic acid sequence encodes ADGRV1 (e.g., a polypeptide represented by UniProt Accession number Q8WXG9 or a polypeptide comprising an amino acid sequence having at least 95% sequence identity thereto).
   1434. The method of embodiment 748, wherein the disease or condition is Febrile Seizure and/or wherein the heterologous nucleic acid sequence encodes CPA6 (e.g., a polypeptide represented by UniProt Accession number P11509 or a polypeptide comprising an amino acid sequence having at least 95% sequence identity thereto).
   1435. The method of embodiment 748, wherein the disease or condition is Febrile Seizure and/or wherein the heterologous nucleic acid sequence encodes SCN1A (e.g., a polypeptide represented by UniProt Accession number P35498 or a polypeptide comprising an amino acid sequence having at least 95% sequence identity thereto).
   1436. The method of embodiment 748, wherein the disease or condition is Friedreich's ataxia and/or wherein the heterologous nucleic acid sequence encodes Frataxin (FXN) (e.g., a polypeptide represented by UniProt Accession number Q16595 or a polypeptide comprising an amino acid sequence having at least 95% sequence identity thereto).
   1437. The method of embodiment 748, wherein the disease or condition is Frontotemporal dementia and/or wherein the heterologous nucleic acid sequence encodes Progranulin (GRN) (e.g., a polypeptide represented by UniProt Accession number P28799 or a polypeptide comprising an amino acid sequence having at least 95% sequence identity thereto).
   1438. The method of embodiment 748, wherein the disease or condition is Frontotemporal dementia and/or wherein the heterologous nucleic acid sequence encodes MAPT (tau) (e.g., a polypeptide represented by UniProt Accession number P10636 or a polypeptide comprising an amino acid sequence having at least 95% sequence identity thereto).
   1439. The method of embodiment 748, wherein the disease or condition is Frontotemporal dementia and/or wherein the heterologous nucleic acid sequence encodes PSEN1 (e.g., a polypeptide represented by UniProt Accession number A0A024R6A3 or a polypeptide comprising an amino acid sequence having at least 95% sequence identity thereto).
   1440. The method of embodiment 748, wherein the disease or condition is Fucosidosis and/or wherein the heterologous nucleic acid sequence encodes alpha-L-fucosidase (FUCA1) (e.g., a polypeptide represented by UniProt Accession number P04066 or a polypeptide comprising an amino acid sequence having at least 95% sequence identity thereto).
   1441. The method of embodiment 748, wherein the disease or condition is Fundus albipunctatus and/or wherein the heterologous nucleic acid sequence encodes RLBP1 (e.g., a polypeptide represented by UniProt Accession number P12271 or a polypeptide comprising an amino acid sequence having at least 95% sequence identity thereto).
   1442. The method of embodiment 748, wherein the disease or condition is Gaucher disease (e.g., types I, II or Ill) and/or wherein the heterologous nucleic acid sequence encodes Glucocerebrosidase (GBA1) (e.g., a polypeptide represented by UniProt Accession number P04062 or a polypeptide comprising an amino acid sequence having at least 95% sequence identity thereto).
   1443. The method of embodiment 748, wherein the disease or condition is Generalized gangliosidose (e.g., GM1, GM2 or GM3) and/or wherein the heterologous nucleic acid sequence encodes GLB1 (e.g., a polypeptide represented by UniProt Accession number P16278 or a polypeptide comprising an amino acid sequence having at least 95% sequence identity thereto).
   1444. The method of embodiment 748, wherein the disease or condition is Gerstmann-Straussler-Scheinker disease and/or wherein the heterologous nucleic acid sequence encodes PRNP (e.g., a polypeptide represented by UniProt Accession number P04156 or a polypeptide comprising an amino acid sequence having at least 95% sequence identity thereto).
   1445. The method of embodiment 748, wherein the disease or condition is Giant axonal neuropathy and/or wherein the heterologous nucleic acid sequence encodes Gigaxonin (GAN) (e.g., a polypeptide represented by UniProt Accession number Q9H2CO or a polypeptide comprising an amino acid sequence having at least 95% sequence identity thereto).
   1446. The method of embodiment 748, wherein the disease or condition is Glycogen storage disease II (Pompe disease or Acid Maltase Deficiency) and/or wherein the heterologous nucleic acid sequence encodes Acid maltase, lysosomal alpha-glucosidase (LYAG, GAA) (e.g., a polypeptide represented by UniProt Accession number P10253 or a polypeptide comprising an amino acid sequence having at least 95% sequence identity thereto).
   1447. The method of embodiment 748, wherein the disease or condition is Guillain-Barre syndrome and/or wherein the heterologous nucleic acid sequence encodes PMP22 (e.g., a polypeptide represented by UniProt Accession number Q01453 or a polypeptide comprising an amino acid sequence having at least 95% sequence identity thereto).
   1448. The method of embodiment 748, wherein the disease or condition is Chronic Inflammatory Demyelinating Polyneuropathy (CIDP) and/or wherein the heterologous nucleic acid sequence encodes PMP22 (e.g., a polypeptide represented by UniProt Accession number Q01453 or a polypeptide comprising an amino acid sequence having at least 95% sequence identity thereto).
   1449. The method of embodiment 748, wherein the disease or condition is Hallervorden-Spatz disease (PKAN or NBIA1) and/or wherein the heterologous nucleic acid sequence encodes PANK2 (e.g., a polypeptide represented by UniProt Accession number Q9BZ23 or a polypeptide comprising an amino acid sequence having at least 95% sequence identity thereto).
   1450. The method of embodiment 748, wherein the disease or condition is Hemiplegia Alterans and/or wherein the heterologous nucleic acid sequence encodes ATP1A2 (e.g., a polypeptide represented by UniProt Accession number P50993 or a polypeptide comprising an amino acid sequence having at least 95% sequence identity thereto).
   1451. The method of embodiment 748, wherein the disease or condition is Hemiplegia Alterans and/or wherein the heterologous nucleic acid sequence encodes ATP1A3 (e.g., a polypeptide represented by UniProt Accession number P13637 or a polypeptide comprising an amino acid sequence having at least 95% sequence identity thereto).
   1452. The method of embodiment 748, wherein the disease or condition is Hereditary Neuropathy and/or wherein the heterologous nucleic acid sequence encodes WNK1 (e.g., a polypeptide represented by UniProt Accession number Q9H4A3 or a polypeptide comprising an amino acid sequence having at least 95% sequence identity thereto).
   1453. The method of embodiment 748, wherein the disease or condition is Hereditary Neuropathy and/or wherein the heterologous nucleic acid sequence encodes MFN2 (e.g., a polypeptide represented by UniProt Accession number 095140 or a polypeptide comprising an amino acid sequence having at least 95% sequence identity thereto).
   1454. The method of embodiment 748, wherein the disease or condition is Hereditary Neuropathy and/or wherein the heterologous nucleic acid sequence encodes HK1 (e.g., a polypeptide represented by UniProt Accession number P19367 or a polypeptide comprising an amino acid sequence having at least 95% sequence identity thereto).
   1455. The method of embodiment 748, wherein the disease or condition is Hereditary Neuropathy and/or wherein the heterologous nucleic acid sequence encodes TFG (e.g., a polypeptide represented by UniProt Accession number Q92734 or a polypeptide comprising an amino acid sequence having at least 95% sequence identity thereto).
   1456. The method of embodiment 748, wherein the disease or condition is Hereditary Neuropathy and/or wherein the heterologous nucleic acid sequence encodes SPTLC1 (e.g., a polypeptide represented by UniProt Accession number 015269 or a polypeptide comprising an amino acid sequence having at least 95% sequence identity thereto).
   1457. The method of embodiment 748, wherein the disease or condition is Heredopathia Atactica Polyneuritiformis (Refsum disease) and/or wherein the heterologous nucleic acid sequence encodes PHYH (e.g., a polypeptide represented by UniProt Accession number 014832 or a polypeptide comprising an amino acid sequence having at least 95% sequence identity thereto).
   1458. The method of embodiment 748, wherein the disease or condition is Holoprosencephaly and/or wherein the heterologous nucleic acid sequence encodes GI12 (e.g., a polypeptide represented by UniProt Accession number P10070 or a polypeptide comprising an amino acid sequence having at least 95% sequence identity thereto).
   1459. The method of embodiment 748, wherein the disease or condition is Holoprosencephaly and/or wherein the heterologous nucleic acid sequence encodes TGIF1 (e.g., a polypeptide represented by UniProt Accession number Q15583 or a polypeptide comprising an amino acid sequence having at least 95% sequence identity thereto).
   1460. The method of embodiment 748, wherein the disease or condition is Holoprosencephaly and/or wherein the heterologous nucleic acid sequence encodes ZIC2 (e.g., a polypeptide represented by UniProt Accession number 095409 or a polypeptide comprising an amino acid sequence having at least 95% sequence identity thereto).
   1461. The method of embodiment 748, wherein the disease or condition is Holoprosencephaly and/or wherein the heterologous nucleic acid sequence encodes PTCH1 (e.g., a polypeptide represented by UniProt Accession number Q13635 or a polypeptide comprising an amino acid sequence having at least 95% sequence identity thereto).
   1462. The method of embodiment 748, wherein the disease or condition is Holoprosencephaly and/or wherein the heterologous nucleic acid sequence encodes SHH (e.g., a polypeptide represented by UniProt Accession number Q15465 or a polypeptide comprising an amino acid sequence having at least 95% sequence identity thereto).
   1463. The method of embodiment 748, wherein the disease or condition is Huntington's disease and/or wherein the heterologous nucleic acid sequence encodes HTT (e.g., a polypeptide represented by UniProt Accession number P42858 or a polypeptide comprising an amino acid sequence having at least 95% sequence identity thereto).
   1464. The method of embodiment 748, wherein the disease or condition is Hydrocephalus disorder and/or wherein the heterologous nucleic acid sequence encodes CCDC88C (e.g., a polypeptide represented by UniProt Accession number Q9P219 or a polypeptide comprising an amino acid sequence having at least 95% sequence identity thereto).
   1465. The method of embodiment 748, wherein the disease or condition is Hydrocephalus disorder and/or wherein the heterologous nucleic acid sequence encodes WDR81 (e.g., a polypeptide represented by UniProt Accession number Q562E7 or a polypeptide comprising an amino acid sequence having at least 95% sequence identity thereto).
   1466. The method of embodiment 748, wherein the disease or condition is Hydrocephalus disorder and/or wherein the heterologous nucleic acid sequence encodes TRIM71 (e.g., a polypeptide represented by UniProt Accession number Q2Q1W2 or a polypeptide comprising an amino acid sequence having at least 95% sequence identity thereto).
   1467. The method of embodiment 748, wherein the disease or condition is Hydrocephalus disorder and/or wherein the heterologous nucleic acid sequence encodes MPDZ (e.g., a polypeptide represented by UniProt Accession number 075970 or a polypeptide comprising an amino acid sequence having at least 95% sequence identity thereto).
   1468. The method of embodiment 748, wherein the disease or condition is Incontinentia Pigmenti and/or wherein the heterologous nucleic acid sequence encodes IKBKG (e.g., a polypeptide represented by UniProt Accession number Q9Y6K9 or a polypeptide comprising an amino acid sequence having at least 95% sequence identity thereto).
   1469. The method of embodiment 748, wherein the disease or condition is Infantile Hypotonia and/or wherein the heterologous nucleic acid sequence encodes NALCN (e.g., a polypeptide represented by UniProt Accession number Q81ZFO or a polypeptide comprising an amino acid sequence having at least 95% sequence identity thereto).
   1470. The method of embodiment 748, wherein the disease or condition is Infantile Hypotonia and/or wherein the heterologous nucleic acid sequence encodes TBCK (e.g., a polypeptide represented by UniProt Accession number Q8TEA7 or a polypeptide comprising an amino acid sequence having at least 95% sequence identity thereto).
   1471. The method of embodiment 748, wherein the disease or condition is Infantile Hypotonia and/or wherein the heterologous nucleic acid sequence encodes CCDCl74 (e.g., a polypeptide represented by UniProt Accession number Q6P113 or a polypeptide comprising an amino acid sequence having at least 95% sequence identity thereto).
   1472. The method of embodiment 748, wherein the disease or condition is Infantile Hypotonia and/or wherein the heterologous nucleic acid sequence encodes UNC80 (e.g., a polypeptide represented by UniProt Accession number Q8N2C7 or a polypeptide comprising an amino acid sequence having at least 95% sequence identity thereto).
   1473. The method of embodiment 748, wherein the disease or condition is Infantile Neuroaxonal Dystrophy and/or wherein the heterologous nucleic acid sequence encodes PLA2G6 (e.g., a polypeptide represented by UniProt Accession number 060733 or a polypeptide comprising an amino acid sequence having at least 95% sequence identity thereto).
   1474. The method of embodiment 748, wherein the disease or condition is Infantile Phytanic Acid Storage Disease (PBD1B) and/or wherein the heterologous nucleic acid sequence encodes PEX1 (e.g., a polypeptide represented by UniProt Accession number 043933 or a polypeptide comprising an amino acid sequence having at least 95% sequence identity thereto).
   1475. The method of embodiment 748, wherein the disease or condition is Joubert Syndrome and/or wherein the heterologous nucleic acid sequence encodes INPP5E (e.g., a polypeptide represented by UniProt Accession number Q9NRR6 or a polypeptide comprising an amino acid sequence having at least 95% sequence identity thereto).
   1476. The method of embodiment 748, wherein the disease or condition is Kennedy Disease and/or wherein the heterologous nucleic acid sequence encodes Androgen receptor (AR) (e.g., a polypeptide represented by UniProt Accession number P10275 or a polypeptide comprising an amino acid sequence having at least 95% sequence identity thereto).
   1477. The method of embodiment 748, wherein the disease or condition is Klippel-Feil Syndrome and/or wherein the heterologous nucleic acid sequence encodes GDF6 (e.g., a polypeptide represented by UniProt Accession number Q6KF10 or a polypeptide comprising an amino acid sequence having at least 95% sequence identity thereto).
   1478. The method of embodiment 748, wherein the disease or condition is Krabbe disease (GALC deficiency) and/or wherein the heterologous nucleic acid sequence encodes GALC (e.g., a polypeptide represented by UniProt Accession number P54803 or a polypeptide comprising an amino acid sequence having at least 95% sequence identity thereto).
   1479. The method of embodiment 748, wherein the disease or condition is Lambert-Eaton Myasthenic Syndrome and/or wherein the heterologous nucleic acid sequence encodes CACNA1A (e.g., a polypeptide represented by UniProt Accession number 000555 or a polypeptide comprising an amino acid sequence having at least 95% sequence identity thereto).
   1480. The method of embodiment 748, wherein the disease or condition is Lambert-Eaton Myasthenic Syndrome and/or wherein the heterologous nucleic acid sequence encodes CACNB2 (e.g., a polypeptide represented by UniProt Accession number Q13936 or a polypeptide comprising an amino acid sequence having at least 95% sequence identity thereto).
   1481. The method of embodiment 748, wherein the disease or condition is Landau-Kleffner Syndrome and/or wherein the heterologous nucleic acid sequence encodes GRIN2A (e.g., a polypeptide represented by UniProt Accession number Q12879 or a polypeptide comprising an amino acid sequence having at least 95% sequence identity thereto).
   1482. The method of embodiment 748, wherein the disease or condition is Late infantile neuronal lipofuscinosis (CLN2) and/or wherein the heterologous nucleic acid sequence encodes TPP1 (e.g., a polypeptide represented by UniProt Accession number 014773 or a polypeptide comprising an amino acid sequence having at least 95% sequence identity thereto).
   1483. The method of embodiment 748, wherein the disease or condition is Lesch-Nyhan Syndrome and/or wherein the heterologous nucleic acid sequence encodes HPRT1 (e.g., a polypeptide represented by UniProt Accession number P00492 or a polypeptide comprising an amino acid sequence having at least 95% sequence identity thereto).
   1484. The method of embodiment 748, wherein the disease or condition is Leber congenital amaurosis (retinal blindness) and/or wherein the heterologous nucleic acid sequence encodes Retinal guanylyl cyclase 1 (GUCY2D) (e.g., a polypeptide represented by UniProt Accession number Q02846 or a polypeptide comprising an amino acid sequence having at least 95% sequence identity thereto).
   1485. The method of embodiment 748, wherein the disease or condition is Leber congenital amaurosis (retinal blindness) and/or wherein the heterologous nucleic acid sequence encodes Retinoid isomerohydrolase (RPE65) (e.g., a polypeptide represented by UniProt Accession number Q16518 or a polypeptide comprising an amino acid sequence having at least 95% sequence identity thereto).
   1486. The method of embodiment 748, wherein the disease or condition is Leber congenital amaurosis (retinal blindness) and/or wherein the heterologous nucleic acid sequence encodes Centrosomal protein of 290 kDa (CEP290) (e.g., a polypeptide represented by UniProt Accession number 015078 or a polypeptide comprising an amino acid sequence having at least 95% sequence identity thereto).
   1487. The method of embodiment 748, wherein the disease or condition is Leber congenital amaurosis (retinal blindness) and/or wherein the heterologous nucleic acid sequence encodes Protein crumbs homolog 1 (CRB1) (e.g., a polypeptide represented by UniProt Accession number P82279 or a polypeptide comprising an amino acid sequence having at least 95% sequence identity thereto).
   1488. The method of embodiment 748, wherein the disease or condition is Leber's hereditary optical neuropathy and/or wherein the heterologous nucleic acid sequence encodes NADH-ubiquinone oxidoreductase chain 4 (ND4) (e.g., a polypeptide represented by UniProt Accession number P03905 or a polypeptide comprising an amino acid sequence having at least 95% sequence identity thereto).
   1489. The method of embodiment 748, wherein the disease or condition is Leukodystrophy and/or wherein the heterologous nucleic acid sequence encodes ARSA (e.g., a polypeptide represented by UniProt Accession number P15289 or a polypeptide comprising an amino acid sequence having at least 95% sequence identity thereto).
   1490. The method of embodiment 748, wherein the disease or condition is Levine-Critchley Syndrome (choreoacanthocytosis) and/or wherein the heterologous nucleic acid sequence encodes VPS13A (e.g., a polypeptide represented by UniProt Accession number Q96RL7 or a polypeptide comprising an amino acid sequence having at least 95% sequence identity thereto).
   1491. The method of embodiment 748, wherein the disease or condition is Lewy body dementia and/or wherein the heterologous nucleic acid sequence encodes SNCA (e.g., a polypeptide represented by UniProt Accession number P37840 or a polypeptide comprising an amino acid sequence having at least 95% sequence identity thereto).
   1492. The method of embodiment 748, wherein the disease or condition is Lewy body dementia and/or wherein the heterologous nucleic acid sequence encodes SNCB (e.g., a polypeptide represented by UniProt Accession number Q16143 or a polypeptide comprising an amino acid sequence having at least 95% sequence identity thereto).
   1493. The method of embodiment 748, wherein the disease or condition is Lipoid Proteinosis (Urbach-Wiethe disease) and/or wherein the heterologous nucleic acid sequence encodes ECM1 (e.g., a polypeptide represented by UniProt Accession number Q16610 or a polypeptide comprising an amino acid sequence having at least 95% sequence identity thereto).
   1494. The method of embodiment 748, wherein the disease or condition is Lissencephaly and/or wherein the heterologous nucleic acid sequence encodes PAFAH1B1 (e.g., a polypeptide represented by UniProt Accession number Q9PTR5 or a polypeptide comprising an amino acid sequence having at least 95% sequence identity thereto).
   1495. The method of embodiment 748, wherein the disease or condition is Lissencephaly and/or wherein the heterologous nucleic acid sequence encodes NDE1 (e.g., a polypeptide represented by UniProt Accession number Q9NXR1 or a polypeptide comprising an amino acid sequence having at least 95% sequence identity thereto).
   1496. The method of embodiment 748, wherein the disease or condition is Lissencephaly and/or wherein the heterologous nucleic acid sequence encodes TUBA1A (e.g., a polypeptide represented by UniProt Accession number Q71U36 or a polypeptide comprising an amino acid sequence having at least 95% sequence identity thereto).
   1497. The method of embodiment 748, wherein the disease or condition is Lissencephaly and/or wherein the heterologous nucleic acid sequence encodes LAMB1 (e.g., a polypeptide represented by UniProt Accession number LAMB1 or a polypeptide comprising an amino acid sequence having at least 95% sequence identity thereto).
   1498. The method of embodiment 748, wherein the disease or condition is Lissencephaly and/or wherein the heterologous nucleic acid sequence encodes KATNB1 (e.g., a polypeptide represented by UniProt Accession number Q9BVA0 or a polypeptide comprising an amino acid sequence having at least 95% sequence identity thereto).
   1499. The method of embodiment 748, wherein the disease or condition is Lissencephaly and/or wherein the heterologous nucleic acid sequence encodes RELN (e.g., a polypeptide represented by UniProt Accession number P78509 or a polypeptide comprising an amino acid sequence having at least 95% sequence identity thereto).
   1500. The method of embodiment 748, wherein the disease or condition is Macrocephaly/Megalencephaly and/or wherein the heterologous nucleic acid sequence encodes TBC1D7 (e.g., a polypeptide represented by UniProt Accession number Q9PON9 or a polypeptide comprising an amino acid sequence having at least 95% sequence identity thereto).
   1501. The method of embodiment 748, wherein the disease or condition is Menkes Disease and/or wherein the heterologous nucleic acid sequence encodes ATP7A (e.g., a polypeptide represented by UniProt Accession number Q04656 or a polypeptide comprising an amino acid sequence having at least 95% sequence identity thereto).
   1502. The method of embodiment 748, wherein the disease or condition is Metachromatic Leukodystrophy (MLD) and/or wherein the heterologous nucleic acid sequence encodes Arylsulfatase A (ARSA) (e.g., a polypeptide represented by UniProt Accession number P15289 or a polypeptide comprising an amino acid sequence having at least 95% sequence identity thereto).
   1503. The method of embodiment 748, wherein the disease or condition is Microcephaly and/or wherein the heterologous nucleic acid sequence encodes KIF11 (e.g., a polypeptide represented by UniProt Accession number P52732 or a polypeptide comprising an amino acid sequence having at least 95% sequence identity thereto).
   1504. The method of embodiment 748, wherein the disease or condition is Microcephaly and/or wherein the heterologous nucleic acid sequence encodes MCPH1 (e.g., a polypeptide represented by UniProt Accession number Q8NEMO or a polypeptide comprising an amino acid sequence having at least 95% sequence identity thereto).
   1505. The method of embodiment 748, wherein the disease or condition is Microcephaly and/or wherein the heterologous nucleic acid sequence encodes SLC25A19 (e.g., a polypeptide represented by UniProt Accession number Q9HC21 or a polypeptide comprising an amino acid sequence having at least 95% sequence identity thereto).
   1506. The method of embodiment 748, wherein the disease or condition is Migraine, familial hemiplegic and/or wherein the heterologous nucleic acid sequence encodes CACNA1A (e.g., a polypeptide represented by UniProt Accession number 000555 or a polypeptide comprising an amino acid sequence having at least 95% sequence identity thereto).
   1507. The method of embodiment 748, wherein the disease or condition is Migraine, familial hemiplegic and/or wherein the heterologous nucleic acid sequence encodes ATP1A2 (e.g., a polypeptide represented by UniProt Accession number P50993 or a polypeptide comprising an amino acid sequence having at least 95% sequence identity thereto).
   1508. The method of embodiment 748, wherein the disease or condition is Migraine, familial hemiplegic and/or wherein the heterologous nucleic acid sequence encodes SCN1A (e.g., a polypeptide represented by UniProt Accession number P35498 or a polypeptide comprising an amino acid sequence having at least 95% sequence identity thereto).
   1509. The method of embodiment 748, wherein the disease or condition is Mitochondrial DNA depletion syndrome and/or wherein the heterologous nucleic acid sequence encodes RRM2B (e.g., a polypeptide represented by UniProt Accession number Q7LG56 or a polypeptide comprising an amino acid sequence having at least 95% sequence identity thereto).
   1510. The method of embodiment 748, wherein the disease or condition is Mitochondrial DNA depletion syndrome and/or wherein the heterologous nucleic acid sequence encodes DGUOK (e.g., a polypeptide represented by UniProt Accession number Q16854 or a polypeptide comprising an amino acid sequence having at least 95% sequence identity thereto).
   1511. The method of embodiment 748, wherein the disease or condition is Mitochondrial DNA depletion syndrome and/or wherein the heterologous nucleic acid sequence encodes POLG (e.g., a polypeptide represented by UniProt Accession number P54098 or a polypeptide comprising an amino acid sequence having at least 95% sequence identity thereto).
   1512. The method of embodiment 748, wherein the disease or condition is Mitochondrial DNA depletion syndrome and/or wherein the heterologous nucleic acid sequence encodes TYMP (e.g., a polypeptide represented by UniProt Accession number P19971 or a polypeptide comprising an amino acid sequence having at least 95% sequence identity thereto).
   1513. The method of embodiment 748, wherein the disease or condition is Mitochondrial DNA depletion syndrome and/or wherein the heterologous nucleic acid sequence encodes TK2 (e.g., a polypeptide represented by UniProt Accession number 000142 or a polypeptide comprising an amino acid sequence having at least 95% sequence identity thereto).
   1514. The method of embodiment 748, wherein the disease or condition is Morvan disease and/or wherein the heterologous nucleic acid sequence encodes WNK1 (e.g., a polypeptide represented by UniProt Accession number Q9H4A3 or a polypeptide comprising an amino acid sequence having at least 95% sequence identity thereto).
   1515. The method of embodiment 748, wherein the disease or condition is Mucolipidosis and/or wherein the heterologous nucleic acid sequence encodes GNPTAB (e.g., a polypeptide represented by UniProt Accession number Q3T906 or a polypeptide comprising an amino acid sequence having at least 95% sequence identity thereto).
   1516. The method of embodiment 748, wherein the disease or condition is Mucolipidosis and/or wherein the heterologous nucleic acid sequence encodes MCOLN1 (e.g., a polypeptide represented by UniProt Accession number Q9GZU1 or a polypeptide comprising an amino acid sequence having at least 95% sequence identity thereto).
   1517. The method of embodiment 748, wherein the disease or condition is Mucopolysaccharidosis Type I (MPS I) (Hurler syndrome) and/or wherein the heterologous nucleic acid sequence encodes alpha-L-iduronidase (IDUA) (e.g., a polypeptide represented by UniProt Accession number P35475 or a polypeptide comprising an amino acid sequence having at least 95% sequence identity thereto).
   1518. The method of embodiment 748, wherein the disease or condition is MPS II (Hunter syndrome) and/or wherein the heterologous nucleic acid sequence encodes iduronate-2-sulfatase (IDS) (e.g., a polypeptide represented by UniProt Accession number P22304 or a polypeptide comprising an amino acid sequence having at least 95% sequence identity thereto).
   1519. The method of embodiment 748, wherein the disease or condition is MPS IIIa (Sanfilippo Type A syndrome) and/or wherein the heterologous nucleic acid sequence encodes heparan sulfate sulfatase (HSS) or N-sulfoglucosamine sulfohydrolase (SGSH) (e.g., a polypeptide represented by UniProt Accession number P51688 or a polypeptide comprising an amino acid sequence having at least 95% sequence identity thereto).
   1520. The method of embodiment 748, wherein the disease or condition is MPS IIIB (Sanfilippo Type B syndrome) and/or wherein the heterologous nucleic acid sequence encodes N-acetyl-alpha-D-glucosaminidase (NAGLU) (e.g., a polypeptide represented by UniProt Accession number P54802 or a polypeptide comprising an amino acid sequence having at least 95% sequence identity thereto).
   1521. The method of embodiment 748, wherein the disease or condition is MPS VI (Maroteaux-Lamy syndrome) and/or wherein the heterologous nucleic acid sequence encodes arylsulfatase B (ARSB) (e.g., a polypeptide represented by UniProt Accession number P15848 or a polypeptide comprising an amino acid sequence having at least 95% sequence identity thereto).
   1522. The method of embodiment 748, wherein the disease or condition is MPS IV A (Morquio syndrome type A) and/or wherein the heterologous nucleic acid sequence encodes N-acetylgalactosamine-6-sulfatase (GALNS) (e.g., a polypeptide represented by UniProt Accession number P34059 or a polypeptide comprising an amino acid sequence having at least 95% sequence identity thereto).
   1523. The method of embodiment 748, wherein the disease or condition is MPS IV B (Morquio syndrome type B) and/or wherein the heterologous nucleic acid sequence encodes Beta-galactosidase 1 (GLB1) (e.g., a polypeptide represented by UniProt Accession number P16278 or a polypeptide comprising an amino acid sequence having at least 95% sequence identity thereto).
   1524. The method of embodiment 748, wherein the disease or condition is MPS VII (Sly syndrome) and/or wherein the heterologous nucleic acid sequence encodes beta-glucuronidase (e.g., a polypeptide represented by UniProt Accession number P08236 or a polypeptide comprising an amino acid sequence having at least 95% sequence identity thereto).
   1525. The method of embodiment 748, wherein the disease or condition is MPS VIII and/or wherein the heterologous nucleic acid sequence encodes glucosamine-6-sulfate sulfatase (e.g., a polypeptide represented by UniProt Accession number P15586 or a polypeptide comprising an amino acid sequence having at least 95% sequence identity thereto).
   1526. The method of embodiment 748, wherein the disease or condition is MPS IX and/or wherein the heterologous nucleic acid sequence encodes Hyaluronidase-1 (HYAL1) (e.g., a polypeptide represented by UniProt Accession number Q12794 or a polypeptide comprising an amino acid sequence having at least 95% sequence identity thereto).
   1527. The method of embodiment 748, wherein the disease or condition is Multiple Sclerosis and/or wherein the heterologous nucleic acid sequence encodes PDCD1 (e.g., a polypeptide represented by UniProt Accession number Q15116 or a polypeptide comprising an amino acid sequence having at least 95% sequence identity thereto).
   1528. The method of embodiment 748, wherein the disease or condition is Multiple system atrophy and/or wherein the heterologous nucleic acid sequence encodes COQ2 (e.g., a polypeptide represented by UniProt Accession number Q96H96 or a polypeptide comprising an amino acid sequence having at least 95% sequence identity thereto).
   1529. The method of embodiment 748, wherein the disease or condition is Myasthenic syndrome, congenital presynaptic and/or wherein the heterologous nucleic acid sequence encodes CHAT (e.g., a polypeptide represented by UniProt Accession number P28329 or a polypeptide comprising an amino acid sequence having at least 95% sequence identity thereto).
   1530. The method of embodiment 748, wherein the disease or condition is Myoclonus and/or wherein the heterologous nucleic acid sequence encodes NOL3 (e.g., a polypeptide represented by UniProt Accession number 060936 or a polypeptide comprising an amino acid sequence having at least 95% sequence identity thereto).
   1531. The method of embodiment 748, wherein the disease or condition is Myoclonic epilepsy (FAME2) and/or wherein the heterologous nucleic acid sequence encodes STARD7 (e.g., a polypeptide represented by UniProt Accession number Q9NQZ5 or a polypeptide comprising an amino acid sequence having at least 95% sequence identity thereto).
   1532. The method of embodiment 748, wherein the disease or condition is Narcolepsy and/or wherein the heterologous nucleic acid sequence encodes HCRT, OX (e.g., a polypeptide represented by UniProt Accession number 043612 or a polypeptide comprising an amino acid sequence having at least 95% sequence identity thereto).
   1533. The method of embodiment 748, wherein the disease or condition is Narcolepsy and/or wherein the heterologous nucleic acid sequence encodes MOG (e.g., a polypeptide represented by UniProt Accession number Q16653 or a polypeptide comprising an amino acid sequence having at least 95% sequence identity thereto).
   1534. The method of embodiment 748, wherein the disease or condition is Neuroacanthocytosis (McLeod syndrome) and/or wherein the heterologous nucleic acid sequence encodes XK (e.g., a polypeptide represented by UniProt Accession number P51811 or a polypeptide comprising an amino acid sequence having at least 95% sequence identity thereto).
   1535. The method of embodiment 748, wherein the disease or condition is Neurodevelopmental disorder with cerebral atrophy and facial dysmorphism (NEDCAFD) and/or wherein the heterologous nucleic acid sequence encodes TTC5 (e.g., a polypeptide represented by UniProt Accession number Q8NOZ6 or a polypeptide comprising an amino acid sequence having at least 95% sequence identity thereto).
   1536. The method of embodiment 748, wherein the disease or condition is Neurodevelopmental disorder with infantile epileptic spasms and/or wherein the heterologous nucleic acid sequence encodes NCDN (e.g., a polypeptide represented by UniProt Accession number Q9UBB6 or a polypeptide comprising an amino acid sequence having at least 95% sequence identity thereto).
   1537. The method of embodiment 748, wherein the disease or condition is Neurofibromatosis and/or wherein the heterologous nucleic acid sequence encodes NF1 (e.g., a polypeptide represented by UniProt Accession number P21359 or a polypeptide comprising an amino acid sequence having at least 95% sequence identity thereto).
   1538. The method of embodiment 748, wherein the disease or condition is Neuromyotonia and/or wherein the heterologous nucleic acid sequence encodes HINT1 (e.g., a polypeptide represented by UniProt Accession number P49773 or a polypeptide comprising an amino acid sequence having at least 95% sequence identity thereto).
   1539. The method of embodiment 748, wherein the disease or condition is Neuronal Ceroid Lipofuscinosis and/or wherein the heterologous nucleic acid sequence encodes PPT1 (e.g., a polypeptide represented by UniProt Accession number P50897 or a polypeptide comprising an amino acid sequence having at least 95% sequence identity thereto).
   1540. The method of embodiment 748, wherein the disease or condition is Neuronal Ceroid Lipofuscinosis and/or wherein the heterologous nucleic acid sequence encodes TPP1 (e.g., a polypeptide represented by UniProt Accession number 014773 or a polypeptide comprising an amino acid sequence having at least 95% sequence identity thereto).
   1541. The method of embodiment 748, wherein the disease or condition is Neuronal Ceroid Lipofuscinosis and/or wherein the heterologous nucleic acid sequence encodes CLN5 (e.g., a polypeptide represented by UniProt Accession number 075503 or a polypeptide comprising an amino acid sequence having at least 95% sequence identity thereto).
   1542. The method of embodiment 748, wherein the disease or condition is Neuronal Ceroid Lipofuscinosis and/or wherein the heterologous nucleic acid sequence encodes CLN3 (e.g., a polypeptide represented by UniProt Accession number Q13286 or a polypeptide comprising an amino acid sequence having at least 95% sequence identity thereto).
   1543. The method of embodiment 748, wherein the disease or condition is Neuronal Ceroid Lipofuscinosis and/or wherein the heterologous nucleic acid sequence encodes CLN6 (e.g., a polypeptide represented by UniProt Accession number Q9NWW5 or a polypeptide comprising an amino acid sequence having at least 95% sequence identity thereto).
   1544. The method of embodiment 748, wherein the disease or condition is Neuronal Ceroid Lipofuscinosis and/or wherein the heterologous nucleic acid sequence encodes CLN8 (e.g., a polypeptide represented by UniProt Accession number Q9UBY8 or a polypeptide comprising an amino acid sequence having at least 95% sequence identity thereto).
   1545. The method of embodiment 748, wherein the disease or condition is Neuronal Ceroid Lipofuscinosis and/or wherein the heterologous nucleic acid sequence encodes DNAJC5 (e.g., a polypeptide represented by UniProt Accession number Q9H3Z4 or a polypeptide comprising an amino acid sequence having at least 95% sequence identity thereto).
   1546. The method of embodiment 748, wherein the disease or condition is Neuronal Ceroid Lipofuscinosis and/or wherein the heterologous nucleic acid sequence encodes MFSD8 (e.g., a polypeptide represented by UniProt Accession number Q8NHS3 or a polypeptide comprising an amino acid sequence having at least 95% sequence identity thereto).
   1547. The method of embodiment 748, wherein the disease or condition is Neuronal Ceroid Lipofuscinosis and/or wherein the heterologous nucleic acid sequence encodes CTSD (e.g., a polypeptide represented by UniProt Accession number P07339 or a polypeptide comprising an amino acid sequence having at least 95% sequence identity thereto).
   1548. The method of embodiment 748, wherein the disease or condition is Neuropathy, ataxia and retinitis pigmentosa (NARP) and/or wherein the heterologous nucleic acid sequence encodes MTATP6 (e.g., a polypeptide represented by UniProt Accession number P00846 or a polypeptide comprising an amino acid sequence having at least 95% sequence identity thereto).
   1549. The method of embodiment 748, wherein the disease or condition is Neuropathy, hereditary sensory and autonomic, type II and/or wherein the heterologous nucleic acid sequence encodes WNK1 (e.g., a polypeptide represented by UniProt Accession number Q9H4A3 or a polypeptide comprising an amino acid sequence having at least 95% sequence identity thereto).
   1550. The method of embodiment 748, wherein the disease or condition is Neuropathy, hypomyelinating congenital 1 and/or wherein the heterologous nucleic acid sequence encodes EGR2 (e.g., a polypeptide represented by UniProt Accession number P11161 or a polypeptide comprising an amino acid sequence having at least 95% sequence identity thereto).
   1551. The method of embodiment 748, wherein the disease or condition is Niemann-Pick disease and/or wherein the heterologous nucleic acid sequence encodes Sphingomyelin phosphodiesterase 1 (SMPD1) (e.g., a polypeptide represented by UniProt Accession number P17405 or a polypeptide comprising an amino acid sequence having at least 95% sequence identity thereto).
   1552. The method of embodiment 748, wherein the disease or condition is Niemann-Pick disease and/or wherein the heterologous nucleic acid sequence encodes NPC intracellular cholesterol transporter 1 (NPC1) (e.g., a polypeptide represented by UniProt Accession number 015118 or a polypeptide comprising an amino acid sequence having at least 95% sequence identity thereto).
   1553. The method of embodiment 748, wherein the disease or condition is Ohtahara Syndrome (Developmental and epileptic encephalopathy 1) and/or wherein the heterologous nucleic acid sequence encodes ARX (e.g., a polypeptide represented by UniProt Accession number Q96QS3 or a polypeptide comprising an amino acid sequence having at least 95% sequence identity thereto).
   1554. The method of embodiment 748, wherein the disease or condition is Omithine Transcarbamylase deficiency and/or wherein the heterologous nucleic acid sequence encodes OTC (e.g., a polypeptide represented by UniProt Accession number P00480 or a polypeptide comprising an amino acid sequence having at least 95% sequence identity thereto).
   1555. The method of embodiment 748, wherein the disease or condition is Orthostatic intolerance and/or wherein the heterologous nucleic acid sequence encodes SLC6A2 (e.g., a polypeptide represented by UniProt Accession number P23975 or a polypeptide comprising an amino acid sequence having at least 95% sequence identity thereto).
   1556. The method of embodiment 748, wherein the disease or condition is Parkinson's disease and/or wherein the heterologous nucleic acid sequence encodes Glucocerebrosidase (GBA1) (e.g., a polypeptide represented by UniProt Accession number P04062 or a polypeptide comprising an amino acid sequence having at least 95% sequence identity thereto).
   1557. The method of embodiment 748, wherein the disease or condition is Parkinson's disease and/or wherein the heterologous nucleic acid sequence encodes Dopamine decarboxylase (DDC) (e.g., a polypeptide represented by UniProt Accession number P20711 or a polypeptide comprising an amino acid sequence having at least 95% sequence identity thereto).
   1558. The method of embodiment 748, wherein the disease or condition is Parkinson's disease and/or wherein the heterologous nucleic acid sequence encodes Neurturin (e.g., a polypeptide represented by UniProt Accession number Q99748 or a polypeptide comprising an amino acid sequence having at least 95% sequence identity thereto).
   1559. The method of embodiment 748, wherein the disease or condition is Parkinson's disease and/or wherein the heterologous nucleic acid sequence encodes Glial derived growth factor (GDGF) (e.g., a polypeptide represented by UniProt Accession number P39905 or a polypeptide comprising an amino acid sequence having at least 95% sequence identity thereto).
   1560. The method of embodiment 748, wherein the disease or condition is Parkinson's disease and/or wherein the heterologous nucleic acid sequence encodes Tyrosine hydroxylase (TH), tyrosine 3-monooxygenase (e.g., a polypeptide represented by UniProt Accession number P07101 or a polypeptide comprising an amino acid sequence having at least 95% sequence identity thereto).
   1561. The method of embodiment 748, wherein the disease or condition is Parkinson's disease and/or wherein the heterologous nucleic acid sequence encodes Glutamic acid decarboxylase (GAD) (e.g., a polypeptide represented by UniProt Accession number Q99259 or a polypeptide comprising an amino acid sequence having at least 95% sequence identity thereto).
   1562. The method of embodiment 748, wherein the disease or condition is Parkinson's disease and/or wherein the heterologous nucleic acid sequence encodes Fibroblast growth factor 2 (FGF2) (e.g., a polypeptide represented by UniProt Accession number P09038 or a polypeptide comprising an amino acid sequence having at least 95% sequence identity thereto).
   1563. The method of embodiment 748, wherein the disease or condition is Parkinson's disease and/or wherein the heterologous nucleic acid sequence encodes Brain-derived neurotrophic factor (BDNF) (e.g., a polypeptide represented by UniProt Accession number P23560 or a polypeptide comprising an amino acid sequence having at least 95% sequence identity thereto).
   1564. The method of embodiment 748, wherein the disease or condition is Paroxysmal Choreoathetosis and/or wherein the heterologous nucleic acid sequence encodes PNKD (e.g., a polypeptide represented by UniProt Accession number Q8N490 or a polypeptide comprising an amino acid sequence having at least 95% sequence identity thereto).
   1565. The method of embodiment 748, wherein the disease or condition is Pelizaeus-Merzbacher Disease and/or wherein the heterologous nucleic acid sequence encodes PLP1 (e.g., a polypeptide represented by UniProt Accession number P60201 or a polypeptide comprising an amino acid sequence having at least 95% sequence identity thereto).
   1566. The method of embodiment 748, wherein the disease or condition is Pena-Shokeir Type II Syndrome and/or wherein the heterologous nucleic acid sequence encodes ERCC6 (e.g., a polypeptide represented by UniProt Accession number Q03468 or a polypeptide comprising an amino acid sequence having at least 95% sequence identity thereto).
   1567. The method of embodiment 748, wherein the disease or condition is Periodic Paralyses and/or wherein the heterologous nucleic acid sequence encodes SCN4A (e.g., a polypeptide represented by UniProt Accession number P35499 or a polypeptide comprising an amino acid sequence having at least 95% sequence identity thereto).
   1568. The method of embodiment 748, wherein the disease or condition is Phelan-McDermid syndrome and/or wherein the heterologous nucleic acid sequence encodes SH3 and multiple ankyrin repeat domains protein 3 (SHANK3) (e.g., a polypeptide represented by UniProt Accession number Q9BYBO or a polypeptide comprising an amino acid sequence having at least 95% sequence identity thereto).
   1569. The method of embodiment 748, wherein the disease or condition is Phytanic Acid Storage Disease (peroxisome biogenesis disorder 1B) and/or wherein the heterologous nucleic acid sequence encodes PEX1 (e.g., a polypeptide represented by UniProt Accession number 043933 or a polypeptide comprising an amino acid sequence having at least 95% sequence identity thereto).
   1570. The method of embodiment 748, wherein the disease or condition is Pick disease and/or wherein the heterologous nucleic acid sequence encodes PSEN1 (e.g., a polypeptide represented by UniProt Accession number A0A024R6A3 or a polypeptide comprising an amino acid sequence having at least 95% sequence identity thereto).
   1571. The method of embodiment 748, wherein the disease or condition is Pick disease and/or wherein the heterologous nucleic acid sequence encodes MAPT (tau) (e.g., a polypeptide represented by UniProt Accession number P10636 or a polypeptide comprising an amino acid sequence having at least 95% sequence identity thereto).
   1572. The method of embodiment 748, wherein the disease or condition is Porencephaly type 1 and/or wherein the heterologous nucleic acid sequence encodes COL4A1 (e.g., a polypeptide represented by UniProt Accession number P02462 or a polypeptide comprising an amino acid sequence having at least 95% sequence identity thereto).
   1573. The method of embodiment 748, wherein the disease or condition is Primary Lateral Sclerosis, juvenile and/or wherein the heterologous nucleic acid sequence encodes ALS2 (e.g., a polypeptide represented by UniProt Accession number Q96Q42 or a polypeptide comprising an amino acid sequence having at least 95% sequence identity thereto).
   1574. The method of embodiment 748, wherein the disease or condition is Primary Progressive Aphasia and/or wherein the heterologous nucleic acid sequence encodes GRN (e.g., a polypeptide represented by UniProt Accession number P28799 or a polypeptide comprising an amino acid sequence having at least 95% sequence identity thereto).
   1575. The method of embodiment 748, wherein the disease or condition is Progressive external ophtalmoplegia and/or wherein the heterologous nucleic acid sequence encodes POLG (e.g., a polypeptide represented by UniProt Accession number P54098 or a polypeptide comprising an amino acid sequence having at least 95% sequence identity thereto).
   1576. The method of embodiment 748, wherein the disease or condition is Progressive external ophtalmoplegia and/or wherein the heterologous nucleic acid sequence encodes POLG2 (e.g., a polypeptide represented by UniProt Accession number Q9UHN1 or a polypeptide comprising an amino acid sequence having at least 95% sequence identity thereto).
   1577. The method of embodiment 748, wherein the disease or condition is Progressive external ophtalmoplegia and/or wherein the heterologous nucleic acid sequence encodes SLC25A4 (e.g., a polypeptide represented by UniProt Accession number P12235 or a polypeptide comprising an amino acid sequence having at least 95% sequence identity thereto).
   1578. The method of embodiment 748, wherein the disease or condition is Progressive external ophtalmoplegia and/or wherein the heterologous nucleic acid sequence encodes TWNK (e.g., a polypeptide represented by UniProt Accession number Q96RR1 or a polypeptide comprising an amino acid sequence having at least 95% sequence identity thereto).
   1579. The method of embodiment 748, wherein the disease or condition is Progressive bulbar palsy and/or wherein the heterologous nucleic acid sequence encodes SLC52A3 (e.g., a polypeptide represented by UniProt Accession number Q9NQ40 or a polypeptide comprising an amino acid sequence having at least 95% sequence identity thereto).
   1580. The method of embodiment 748, wherein the disease or condition is Progressive supranuclear palsy and/or wherein the heterologous nucleic acid sequence encodes Microtubule-associated protein tau (MAPT), Tau (e.g., a polypeptide represented by UniProt Accession number P10636 or a polypeptide comprising an amino acid sequence having at least 95% sequence identity thereto).
   1581. The method of embodiment 748, wherein the disease or condition is Pseudo-Torch syndrome and/or wherein the heterologous nucleic acid sequence encodes OCLN (e.g., a polypeptide represented by UniProt Accession number Q16625 or a polypeptide comprising an amino acid sequence having at least 95% sequence identity thereto).
   1582. The method of embodiment 748, wherein the disease or condition is Pseudo-Torch syndrome and/or wherein the heterologous nucleic acid sequence encodes STAT2 (e.g., a polypeptide represented by UniProt Accession number P52630 or a polypeptide comprising an amino acid sequence having at least 95% sequence identity thereto).
   1583. The method of embodiment 748, wherein the disease or condition is Pseudo-Torch syndrome and/or wherein the heterologous nucleic acid sequence encodes USP18 (e.g., a polypeptide represented by UniProt Accession number Q9UMW8 or a polypeptide comprising an amino acid sequence having at least 95% sequence identity thereto).
   1584. The method of embodiment 748, wherein the disease or condition is Refsum Disease and/or wherein the heterologous nucleic acid sequence encodes PHYH (e.g., a polypeptide represented by UniProt Accession number 014832 or a polypeptide comprising an amino acid sequence having at least 95% sequence identity thereto).
   1585. The method of embodiment 748, wherein the disease or condition is Retinitis Pigmentosa 38 (rod-cone dystrophy) and/or wherein the heterologous nucleic acid sequence encodes Tyrosine-protein kinase Mer (MERTK) (e.g., a polypeptide represented by UniProt Accession number Q12866 or a polypeptide comprising an amino acid sequence having at least 95% sequence identity thereto).
   1586. The method of embodiment 748, wherein the disease or condition is Retinitis Pigmentosa 40 and/or wherein the heterologous nucleic acid sequence encodes PDE6B (e.g., a polypeptide represented by UniProt Accession number P35913 or a polypeptide comprising an amino acid sequence having at least 95% sequence identity thereto).
   1587. The method of embodiment 748, wherein the disease or condition is Rett syndrome and/or wherein the heterologous nucleic acid sequence encodes Methyl-CpG-binding protein 2 (MECP2) (e.g., a polypeptide represented by UniProt Accession number P51608 or a polypeptide comprising an amino acid sequence having at least 95% sequence identity thereto).
   1588. The method of embodiment 748, wherein the disease or condition is Sandhoff disease and/or wherein the heterologous nucleic acid sequence encodes Beta-hexosaminidase subunit alpha (HEXA) (e.g., a polypeptide represented by UniProt Accession number P06865 or a polypeptide comprising an amino acid sequence having at least 95% sequence identity thereto).
   1589. The method of embodiment 748, wherein the disease or condition is Sandhoff disease and/or wherein the heterologous nucleic acid sequence encodes Beta-hexosaminidase subunit beta (HEXB) (e.g., a polypeptide represented by UniProt Accession number P07686 or a polypeptide comprising an amino acid sequence having at least 95% sequence identity thereto).
   1590. The method of embodiment 748, wherein the disease or condition is Schizencephaly and/or wherein the heterologous nucleic acid sequence encodes SIX3 (e.g., a polypeptide represented by UniProt Accession number 095343 or a polypeptide comprising an amino acid sequence having at least 95% sequence identity thereto).
   1591. The method of embodiment 748, wherein the disease or condition is Schizencephaly and/or wherein the heterologous nucleic acid sequence encodes EMX2 (e.g., a polypeptide represented by UniProt Accession number Q04743 or a polypeptide comprising an amino acid sequence having at least 95% sequence identity thereto).
   1592. The method of embodiment 748, wherein the disease or condition is Schizencephaly and/or wherein the heterologous nucleic acid sequence encodes SHH (e.g., a polypeptide represented by UniProt Accession number Q15465 or a polypeptide comprising an amino acid sequence having at least 95% sequence identity thereto).
   1593. The method of embodiment 748, wherein the disease or condition is Seitelberger Disease and/or wherein the heterologous nucleic acid sequence encodes PLA2G6 (e.g., a polypeptide represented by UniProt Accession number 060733 or a polypeptide comprising an amino acid sequence having at least 95% sequence identity thereto).
   1594. The method of embodiment 748, wherein the disease or condition is Septo-optic dysplasia (De Morsier syndrome) and/or wherein the heterologous nucleic acid sequence encodes HESX1 (e.g., a polypeptide represented by UniProt Accession number Q9UBX0 or a polypeptide comprising an amino acid sequence having at least 95% sequence identity thereto).
   1595. The method of embodiment 748, wherein the disease or condition is Snijders Blok-Fisher syndrome and/or wherein the heterologous nucleic acid sequence encodes POU3F3 (e.g., a polypeptide represented by UniProt Accession number P20264 or a polypeptide comprising an amino acid sequence having at least 95% sequence identity thereto).
   1596. The method of embodiment 748, wherein the disease or condition is Spastic Paraplegias and/or wherein the heterologous nucleic acid sequence encodes SPG11 (e.g., a polypeptide represented by UniProt Accession number Q96J17 or a polypeptide comprising an amino acid sequence having at least 95% sequence identity thereto).
   1597. The method of embodiment 748, wherein the disease or condition is Spastic Paraplegias and/or wherein the heterologous nucleic acid sequence encodes SPAST (e.g., a polypeptide represented by UniProt Accession number Q9UBP0 or a polypeptide comprising an amino acid sequence having at least 95% sequence identity thereto).
   1598. The method of embodiment 748, wherein the disease or condition is Spastic Paraplegias and/or wherein the heterologous nucleic acid sequence encodes KIF5A (e.g., a polypeptide represented by UniProt Accession number Q12840 or a polypeptide comprising an amino acid sequence having at least 95% sequence identity thereto).
   1599. The method of embodiment 748, wherein the disease or condition is Spastic Paraplegias and/or wherein the heterologous nucleic acid sequence encodes NIPA1 (e.g., a polypeptide represented by UniProt Accession number Q7RTP0 or a polypeptide comprising an amino acid sequence having at least 95% sequence identity thereto).
   1600. The method of embodiment 748, wherein the disease or condition is Spastic Paraplegias and/or wherein the heterologous nucleic acid sequence encodes CYP7B1 (e.g., a polypeptide represented by UniProt Accession number 075881 or a polypeptide comprising an amino acid sequence having at least 95% sequence identity thereto).
   1601. The method of embodiment 748, wherein the disease or condition is Spastic Paraplegias and/or wherein the heterologous nucleic acid sequence encodes ATL1 (e.g., a polypeptide represented by UniProt Accession number Q8WXF7 or a polypeptide comprising an amino acid sequence having at least 95% sequence identity thereto).
   1602. The method of embodiment 748, wherein the disease or condition is Spinal Muscular Atrophy (Kugelberg-Welander Disease) and/or wherein the heterologous nucleic acid sequence encodes Survival motor neuron protein (SMN), SMN1 (e.g., a polypeptide represented by UniProt Accession number Q16637 or a polypeptide comprising an amino acid sequence having at least 95% sequence identity thereto).
   1603. The method of embodiment 748, wherein the disease or condition is Spinocerebellar ataxia and/or wherein the heterologous nucleic acid sequence encodes Ataxin-1 (ATXN1), SCA1 (e.g., a polypeptide represented by UniProt Accession number P54253 or a polypeptide comprising an amino acid sequence having at least 95% sequence identity thereto).
   1604. The method of embodiment 748, wherein the disease or condition is Spinocerebellar ataxia and/or wherein the heterologous nucleic acid sequence encodes Ataxin-2 (ATXN2), SCA2 (e.g., a polypeptide represented by UniProt Accession number Q99700 or a polypeptide comprising an amino acid sequence having at least 95% sequence identity thereto).
   1605. The method of embodiment 748, wherein the disease or condition is Spinocerebellar ataxia and/or wherein the heterologous nucleic acid sequence encodes Ataxin-3 (ATXN3), SCA3 (e.g., a polypeptide represented by UniProt Accession number P54252 or a polypeptide comprising an amino acid sequence having at least 95% sequence identity thereto).
   1606. The method of embodiment 748, wherein the disease or condition is Spinocerebellar ataxia and/or wherein the heterologous nucleic acid sequence encodes ZFHX3 (e.g., a polypeptide represented by UniProt Accession number Q15911 or a polypeptide comprising an amino acid sequence having at least 95% sequence identity thereto).
   1607. The method of embodiment 748, wherein the disease or condition is Spinocerebellar ataxia and/or wherein the heterologous nucleic acid sequence encodes CACNA1A (e.g., a polypeptide represented by UniProt Accession number 000555 or a polypeptide comprising an amino acid sequence having at least 95% sequence identity thereto).
   1608. The method of embodiment 748, wherein the disease or condition is Spinocerebellar ataxia and/or wherein the heterologous nucleic acid sequence encodes ATXN7, SCA7 (e.g., a polypeptide represented by UniProt Accession number 015265 or a polypeptide comprising an amino acid sequence having at least 95% sequence identity thereto).
   1609. The method of embodiment 748, wherein the disease or condition is Spinocerebellar ataxia and/or wherein the heterologous nucleic acid sequence encodes TMEM240 (e.g., a polypeptide represented by UniProt Accession number Q5SV17 or a polypeptide comprising an amino acid sequence having at least 95% sequence identity thereto).
   1610. The method of embodiment 748, wherein the disease or condition is Sporadic Inclusion Body Myositis and/or wherein the heterologous nucleic acid sequence encodes Follistatin (FST) (e.g., a polypeptide represented by UniProt Accession number P19883 or a polypeptide comprising an amino acid sequence having at least 95% sequence identity thereto).
   1611. The method of embodiment 748, wherein the disease or condition is Steele-Richardson-Olszewski syndrome (Parkinson-dementia syndrome) and/or wherein the heterologous nucleic acid sequence encodes MAPT(Tau) (e.g., a polypeptide represented by UniProt Accession number P10636 or a polypeptide comprising an amino acid sequence having at least 95% sequence identity thereto).
   1612. The method of embodiment 748, wherein the disease or condition is Stiff-Person Syndrome, congenital and/or wherein the heterologous nucleic acid sequence encodes GLRA1 (e.g., a polypeptide represented by UniProt Accession number P23415 or a polypeptide comprising an amino acid sequence having at least 95% sequence identity thereto).
   1613. The method of embodiment 748, wherein the disease or condition is Stiff-Person Syndrome, congenital and/or wherein the heterologous nucleic acid sequence encodes GLRB (e.g., a polypeptide represented by UniProt Accession number P48167 or a polypeptide comprising an amino acid sequence having at least 95% sequence identity thereto).
   1614. The method of embodiment 748, wherein the disease or condition is Striatonigral degeneration and/or wherein the heterologous nucleic acid sequence encodes NUP62 (e.g., a polypeptide represented by UniProt Accession number P37198 or a polypeptide comprising an amino acid sequence having at least 95% sequence identity thereto).
   1615. The method of embodiment 748, wherein the disease or condition is Striatonigral degeneration and/or wherein the heterologous nucleic acid sequence encodes PDE8B (e.g., a polypeptide represented by UniProt Accession number 095263 or a polypeptide comprising an amino acid sequence having at least 95% sequence identity thereto).
   1616. The method of embodiment 748, wherein the disease or condition is Striatonigral degeneration and/or wherein the heterologous nucleic acid sequence encodes MTATP6 (e.g., a polypeptide represented by UniProt Accession number P00846 or a polypeptide comprising an amino acid sequence having at least 95% sequence identity thereto).
   1617. The method of embodiment 748, wherein the disease or condition is Striatonigral degeneration and/or wherein the heterologous nucleic acid sequence encodes VAC14 (e.g., a polypeptide represented by UniProt Accession number Q08AM6 or a polypeptide comprising an amino acid sequence having at least 95% sequence identity thereto).
   1618. The method of embodiment 748, wherein the disease or condition is Sturge-Weber Syndrome and/or wherein the heterologous nucleic acid sequence encodes GNAQ (e.g., a polypeptide represented by UniProt Accession number P50148 or a polypeptide comprising an amino acid sequence having at least 95% sequence identity thereto).
   1619. The method of embodiment 748, wherein the disease or condition is Subcortical Vascular Encephalopathy (Cerebral Arteriopathy) and/or wherein the heterologous nucleic acid sequence encodes HTRA1 (e.g., a polypeptide represented by UniProt Accession number Q92743 or a polypeptide comprising an amino acid sequence having at least 95% sequence identity thereto).
   1620. The method of embodiment 748, wherein the disease or condition is Systemic Lupus Erythematosus and/or wherein the heterologous nucleic acid sequence encodes DNASE1L3 (e.g., a polypeptide represented by UniProt Accession number Q13609 or a polypeptide comprising an amino acid sequence having at least 95% sequence identity thereto).
   1621. The method of embodiment 748, wherein the disease or condition is Systemic Lupus Erythematosus and/or wherein the heterologous nucleic acid sequence encodes TLR7 (e.g., a polypeptide represented by UniProt Accession number Q9NYK1 or a polypeptide comprising an amino acid sequence having at least 95% sequence identity thereto).
   1622. The method of embodiment 748, wherein the disease or condition is Tardive Dyskinesia and/or wherein the heterologous nucleic acid sequence encodes CYP2D6 (e.g., a polypeptide represented by UniProt Accession number P10635 or a polypeptide comprising an amino acid sequence having at least 95% sequence identity thereto).
   1623. The method of embodiment 748, wherein the disease or condition is Tay-Sachs disease and/or wherein the heterologous nucleic acid sequence encodes Beta-hexosaminidase subunit alpha (HEXA) (e.g., a polypeptide represented by UniProt Accession number P06865 or a polypeptide comprising an amino acid sequence having at least 95% sequence identity thereto).
   1624. The method of embodiment 748, wherein the disease or condition is Tourette Syndrome and/or wherein the heterologous nucleic acid sequence encodes HDC (e.g., a polypeptide represented by UniProt Accession number P19113 or a polypeptide comprising an amino acid sequence having at least 95% sequence identity thereto).
   1625. The method of embodiment 748, wherein the disease or condition is Tourette Syndrome and/or wherein the heterologous nucleic acid sequence encodes SLITRK1 (e.g., a polypeptide represented by UniProt Accession number Q96PX8 or a polypeptide comprising an amino acid sequence having at least 95% sequence identity thereto).
   1626. The method of embodiment 748, wherein the disease or condition is Tremor, hereditary type 1 and/or wherein the heterologous nucleic acid sequence encodes DRD3 (e.g., a polypeptide represented by UniProt Accession number P35462 or a polypeptide comprising an amino acid sequence having at least 95% sequence identity thereto).
   1627. The method of embodiment 748, wherein the disease or condition is Troyer Syndrome and/or wherein the heterologous nucleic acid sequence encodes SPART (e.g., a polypeptide represented by UniProt Accession number Q8N0X₇ or a polypeptide comprising an amino acid sequence having at least 95% sequence identity thereto).
   1628. The method of embodiment 748, wherein the disease or condition is Tuberous Sclerosis and/or wherein the heterologous nucleic acid sequence encodes TSC1 (e.g., a polypeptide represented by UniProt Accession number Q92574 or a polypeptide comprising an amino acid sequence having at least 95% sequence identity thereto).
   1629. The method of embodiment 748, wherein the disease or condition is Tuberous Sclerosis and/or wherein the heterologous nucleic acid sequence encodes TSC2 (e.g., a polypeptide represented by UniProt Accession number P49815 or a polypeptide comprising an amino acid sequence having at least 95% sequence identity thereto).
   1630. The method of embodiment 748, wherein the disease or condition is Tuberous Sclerosis and/or wherein the heterologous nucleic acid sequence encodes IFNG (e.g., a polypeptide represented by UniProt Accession number P01579 or a polypeptide comprising an amino acid sequence having at least 95% sequence identity thereto).
   1631. The method of embodiment 748, wherein the disease or condition is Von Hippel-Lindau Disease and/or wherein the heterologous nucleic acid sequence encodes VHL (e.g., a polypeptide represented by UniProt Accession number P40337 or a polypeptide comprising an amino acid sequence having at least 95% sequence identity thereto).
   1632. The method of embodiment 748, wherein the disease or condition is Von Hippel-Lindau Disease and/or wherein the heterologous nucleic acid sequence encodes CCND1 (e.g., a polypeptide represented by UniProt Accession number P24385 or a polypeptide comprising an amino acid sequence having at least 95% sequence identity thereto).
   1633. The method of embodiment 748, wherein the disease or condition is Von Recklinghausen Disease and/or wherein the heterologous nucleic acid sequence encodes NF1 (e.g., a polypeptide represented by UniProt Accession number P21359 or a polypeptide comprising an amino acid sequence having at least 95% sequence identity thereto).
   1634. The method of embodiment 748, wherein the disease or condition is Werdnig-Hoffman Disease and/or wherein the heterologous nucleic acid sequence encodes SMN1 (e.g., a polypeptide represented by UniProt Accession number Q16637 or a polypeptide comprising an amino acid sequence having at least 95% sequence identity thereto).
   1635. The method of embodiment 748, wherein the disease or condition is West Syndrome, X-linked and/or wherein the heterologous nucleic acid sequence encodes ARX (e.g., a polypeptide represented by UniProt Accession number Q96QS3 or a polypeptide comprising an amino acid sequence having at least 95% sequence identity thereto).
   1636. The method of embodiment 748, wherein the disease or condition is Wilson disease and/or wherein the heterologous nucleic acid sequence encodes ATP7B (e.g., a polypeptide represented by UniProt Accession number P35670 or a polypeptide comprising an amino acid sequence having at least 95% sequence identity thereto).
   1637. The method of embodiment 748, wherein the disease or condition is Wolman's disease (acid lipase disease) and/or wherein the heterologous nucleic acid sequence encodes LIPA (e.g., a polypeptide represented by UniProt Accession number P38571 or a polypeptide comprising an amino acid sequence having at least 95% sequence identity thereto).
   1638. The method of embodiment 748, wherein the disease or condition is X-linked adrenoleukodystrophy and/or wherein the heterologous nucleic acid sequence encodes ATP-binding cassette sub-family D member 1 (ABCD1) (e.g., a polypeptide represented by UniProt Accession number P33897 or a polypeptide comprising an amino acid sequence having at least 95% sequence identity thereto).
   1639. The method of embodiment 748, wherein the disease or condition is X-linked Retinoschisis and/or wherein the heterologous nucleic acid sequence encodes Retinoschisin (RS1) (e.g., a polypeptide represented by UniProt Accession number 015537 or a polypeptide comprising an amino acid sequence having at least 95% sequence identity thereto).
   1640. The method of embodiment 748, wherein the disease or condition is X-Linked Retinitis Pigmentosa and/or wherein the heterologous nucleic acid sequence encodes X-linked retinitis pigmentosa GTPase regulator (RPGR) (e.g., a polypeptide represented by UniProt Accession number Q92834 or a polypeptide comprising an amino acid sequence having at least 95% sequence identity thereto).
   1641. The method of embodiment 748, wherein the disease or condition is X-Linked Spinal and Bulbar Muscular Atrophy and/or wherein the heterologous nucleic acid sequence encodes UBA1 (e.g., a polypeptide represented by UniProt Accession number P22314 or a polypeptide comprising an amino acid sequence having at least 95% sequence identity thereto).
   1642. The method of embodiment 748, wherein the disease or condition is Advanced heart failure and/or wherein the heterologous nucleic acid sequence encodes SERCA2a, ATP2A2 (e.g., a polypeptide represented by UniProt Accession number P16615 or a polypeptide comprising an amino acid sequence having at least 95% sequence identity thereto).
   1643. The method of embodiment 748, wherein the disease or condition is Amyotrophic lateral sclerosis (ALS) (Lou Gehrig's disease) and/or wherein the heterologous nucleic acid sequence encodes Superoxide dismutase-1 (SOD1) (e.g., a polypeptide represented by UniProt Accession number P00441 or a polypeptide comprising an amino acid sequence having at least 95% sequence identity thereto).
   1644. The method of embodiment 748, wherein the disease or condition is Andersen-Tawil Syndrome and/or wherein the heterologous nucleic acid sequence encodes KCNJ2 (e.g., a polypeptide represented by UniProt Accession number P63252 or a polypeptide comprising an amino acid sequence having at least 95% sequence identity thereto).
   1645. The method of embodiment 748, wherein the disease or condition is Barth syndrome and/or wherein the heterologous nucleic acid sequence encodes TAFAZZIN (e.g., a polypeptide represented by UniProt Accession number Q16635 or a polypeptide comprising an amino acid sequence having at least 95% sequence identity thereto).
   1646. The method of embodiment 748, wherein the disease or condition is Becker Muscular Dystrophy (BMD) and/or wherein the heterologous nucleic acid sequence encodes DMD (e.g., a polypeptide represented by UniProt Accession number P11532 or a polypeptide comprising an amino acid sequence having at least 95% sequence identity thereto).
   1647. The method of embodiment 748, wherein the disease or condition is Becker Myotonia Congenita and/or wherein the heterologous nucleic acid sequence encodes CLCN1 (e.g., a polypeptide represented by UniProt Accession number P35523 or a polypeptide comprising an amino acid sequence having at least 95% sequence identity thereto).
   1648. The method of embodiment 748, wherein the disease or condition is Bethlem Myopathy and/or wherein the heterologous nucleic acid sequence encodes COL6A3 (e.g., a polypeptide represented by UniProt Accession number P12111 or a polypeptide comprising an amino acid sequence having at least 95% sequence identity thereto).
   1649. The method of embodiment 748, wherein the disease or condition is Bethlem Myopathy and/or wherein the heterologous nucleic acid sequence encodes COL6A2 (e.g., a polypeptide represented by UniProt Accession number P12110 or a polypeptide comprising an amino acid sequence having at least 95% sequence identity thereto).
   1650. The method of embodiment 748, wherein the disease or condition is Bethlem Myopathy and/or wherein the heterologous nucleic acid sequence encodes COL6A1 (e.g., a polypeptide represented by UniProt Accession number P12109 or a polypeptide comprising an amino acid sequence having at least 95% sequence identity thereto).
   1651. The method of embodiment 748, wherein the disease or condition is Bulbospinal Muscular Atrophy and/or wherein the heterologous nucleic acid sequence encodes AR (e.g., a polypeptide represented by UniProt Accession number P10275 or a polypeptide comprising an amino acid sequence having at least 95% sequence identity thereto).
   1652. The method of embodiment 748, wherein the disease or condition is Carnitine Deficiency, systemic primary and/or wherein the heterologous nucleic acid sequence encodes SLC22A5 (e.g., a polypeptide represented by UniProt Accession number 076082 or a polypeptide comprising an amino acid sequence having at least 95% sequence identity thereto).
   1653. The method of embodiment 748, wherein the disease or condition is Carnitine Palmityl Transferase Deficiency, type 1 and/or wherein the heterologous nucleic acid sequence encodes CPT1A (e.g., a polypeptide represented by UniProt Accession number P50416 or a polypeptide comprising an amino acid sequence having at least 95% sequence identity thereto).
   1654. The method of embodiment 748, wherein the disease or condition is Carnitine Palmityl Transferase Deficiency, type 2 and/or wherein the heterologous nucleic acid sequence encodes CPT2 (e.g., a polypeptide represented by UniProt Accession number P23786 or a polypeptide comprising an amino acid sequence having at least 95% sequence identity thereto).
   1655. The method of embodiment 748, wherein the disease or condition is Catecholaminergic polymorphic ventricular tachycardia 2 (CPVT2) and/or wherein the heterologous nucleic acid sequence encodes Calsequestrin-2 (CASQ2) (e.g., a polypeptide represented by UniProt Accession number 014958 or a polypeptide comprising an amino acid sequence having at least 95% sequence identity thereto).
   1656. The method of embodiment 748, wherein the disease or condition is Central Core Disease (congenital myopathy, type 1A) and/or wherein the heterologous nucleic acid sequence encodes RYR1 (e.g., a polypeptide represented by UniProt Accession number P21817 or a polypeptide comprising an amino acid sequence having at least 95% sequence identity thereto).
   1657. The method of embodiment 748, wherein the disease or condition is Centronuclear Myopathy type 1 and/or wherein the heterologous nucleic acid sequence encodes MTMR14 (e.g., a polypeptide represented by UniProt Accession number Q8NCE2 or a polypeptide comprising an amino acid sequence having at least 95% sequence identity thereto).
   1658. The method of embodiment 748, wherein the disease or condition is Centronuclear Myopathy type 2 and/or wherein the heterologous nucleic acid sequence encodes DNM2 (e.g., a polypeptide represented by UniProt Accession number 014717 or a polypeptide comprising an amino acid sequence having at least 95% sequence identity thereto).
   1659. The method of embodiment 748, wherein the disease or condition is Charcot-Marie-Tooth disease and/or wherein the heterologous nucleic acid sequence encodes PMP22 (e.g., a polypeptide represented by UniProt Accession number Q01453 or a polypeptide comprising an amino acid sequence having at least 95% sequence identity thereto).
   1660. The method of embodiment 748, wherein the disease or condition is Charcot-Marie-Tooth disease and/or wherein the heterologous nucleic acid sequence encodes MPZ (e.g., a polypeptide represented by UniProt Accession number P25189 or a polypeptide comprising an amino acid sequence having at least 95% sequence identity thereto).
   1661. The method of embodiment 748, wherein the disease or condition is Charcot-Marie-Tooth disease and/or wherein the heterologous nucleic acid sequence encodes DNM2 (e.g., a polypeptide represented by UniProt Accession number P50570 or a polypeptide comprising an amino acid sequence having at least 95% sequence identity thereto).
   1662. The method of embodiment 748, wherein the disease or condition is Charcot-Marie-Tooth disease and/or wherein the heterologous nucleic acid sequence encodes MFN2 (e.g., a polypeptide represented by UniProt Accession number 095140 or a polypeptide comprising an amino acid sequence having at least 95% sequence identity thereto).
   1663. The method of embodiment 748, wherein the disease or condition is Charcot-Marie-Tooth disease and/or wherein the heterologous nucleic acid sequence encodes KIF1B (e.g., a polypeptide represented by UniProt Accession number 060333 or a polypeptide comprising an amino acid sequence having at least 95% sequence identity thereto).
   1664. The method of embodiment 748, wherein the disease or condition is Charcot-Marie-Tooth disease and/or wherein the heterologous nucleic acid sequence encodes SBF2 (e.g., a polypeptide represented by UniProt Accession number Q86WG5 or a polypeptide comprising an amino acid sequence having at least 95% sequence identity thereto).
   1665. The method of embodiment 748, wherein the disease or condition is Charcot-Marie-Tooth disease and/or wherein the heterologous nucleic acid sequence encodes PNKP (e.g., a polypeptide represented by UniProt Accession number Q96T60 or a polypeptide comprising an amino acid sequence having at least 95% sequence identity thereto).
   1666. The method of embodiment 748, wherein the disease or condition is Charcot-Marie-Tooth disease and/or wherein the heterologous nucleic acid sequence encodes GDAP1 (e.g., a polypeptide represented by UniProt Accession number Q8TB36 or a polypeptide comprising an amino acid sequence having at least 95% sequence identity thereto).
   1667. The method of embodiment 748, wherein the disease or condition is Charcot-Marie-Tooth disease and/or wherein the heterologous nucleic acid sequence encodes LMNA (e.g., a polypeptide represented by UniProt Accession number P02545 or a polypeptide comprising an amino acid sequence having at least 95% sequence identity thereto).
   1668. The method of embodiment 748, wherein the disease or condition is Charcot-Marie-Tooth disease and/or wherein the heterologous nucleic acid sequence encodes FGD4 (e.g., a polypeptide represented by UniProt Accession number Q96M96 or a polypeptide comprising an amino acid sequence having at least 95% sequence identity thereto).
   1669. The method of embodiment 748, wherein the disease or condition is Charcot-Marie-Tooth disease and/or wherein the heterologous nucleic acid sequence encodes MTMR2 (e.g., a polypeptide represented by UniProt Accession number Q13614 or a polypeptide comprising an amino acid sequence having at least 95% sequence identity thereto).
   1670. The method of embodiment 748, wherein the disease or condition is Congenital Muscular Dystrophy (Ullrich disease) and/or wherein the heterologous nucleic acid sequence encodes COL6A3 (e.g., a polypeptide represented by UniProt Accession number P12111 or a polypeptide comprising an amino acid sequence having at least 95% sequence identity thereto).
   1671. The method of embodiment 748, wherein the disease or condition is Congenital Muscular Dystrophy (Ullrich disease) and/or wherein the heterologous nucleic acid sequence encodes COL6A2 (e.g., a polypeptide represented by UniProt Accession number P12110 or a polypeptide comprising an amino acid sequence having at least 95% sequence identity thereto).
   1672. The method of embodiment 748, wherein the disease or condition is Congenital Muscular Dystrophy (Ullrich disease) and/or wherein the heterologous nucleic acid sequence encodes COL6A1 (e.g., a polypeptide represented by UniProt Accession number P12109 or a polypeptide comprising an amino acid sequence having at least 95% sequence identity thereto).
   1673. The method of embodiment 748, wherein the disease or condition is Congenital Myasthenic Syndrome and/or wherein the heterologous nucleic acid sequence encodes COLQ (e.g., a polypeptide represented by UniProt Accession number Q9Y215 or a polypeptide comprising an amino acid sequence having at least 95% sequence identity thereto).
   1674. The method of embodiment 748, wherein the disease or condition is Congenital Myasthenic Syndrome and/or wherein the heterologous nucleic acid sequence encodes AGRN (e.g., a polypeptide represented by UniProt Accession number 000468 or a polypeptide comprising an amino acid sequence having at least 95% sequence identity thereto).
   1675. The method of embodiment 748, wherein the disease or condition is Congenital Myasthenic Syndrome and/or wherein the heterologous nucleic acid sequence encodes RAPSN (e.g., a polypeptide represented by UniProt Accession number Q13702 or a polypeptide comprising an amino acid sequence having at least 95% sequence identity thereto).
   1676. The method of embodiment 748, wherein the disease or condition is Congenital Myasthenic Syndrome and/or wherein the heterologous nucleic acid sequence encodes GFPT1 (e.g., a polypeptide represented by UniProt Accession number Q06210 or a polypeptide comprising an amino acid sequence having at least 95% sequence identity thereto).
   1677. The method of embodiment 748, wherein the disease or condition is Congenital Myasthenic Syndrome and/or wherein the heterologous nucleic acid sequence encodes SCN4A (e.g., a polypeptide represented by UniProt Accession number P35499 or a polypeptide comprising an amino acid sequence having at least 95% sequence identity thereto).
   1678. The method of embodiment 748, wherein the disease or condition is Congenital Myasthenic Syndrome and/or wherein the heterologous nucleic acid sequence encodes ALG2 (e.g., a polypeptide represented by UniProt Accession number Q9H553 or a polypeptide comprising an amino acid sequence having at least 95% sequence identity thereto).
   1679. The method of embodiment 748, wherein the disease or condition is Congenital Myasthenic Syndrome and/or wherein the heterologous nucleic acid sequence encodes ALG14 (e.g., a polypeptide represented by UniProt Accession number Q96F25 or a polypeptide comprising an amino acid sequence having at least 95% sequence identity thereto).
   1680. The method of embodiment 748, wherein the disease or condition is Congenital Myasthenic Syndrome and/or wherein the heterologous nucleic acid sequence encodes DPAGT1 (e.g., a polypeptide represented by UniProt Accession number Q9H3H5 or a polypeptide comprising an amino acid sequence having at least 95% sequence identity thereto).
   1681. The method of embodiment 748, wherein the disease or condition is Congenital Myasthenic Syndrome and/or wherein the heterologous nucleic acid sequence encodes CHRNE (e.g., a polypeptide represented by UniProt Accession number Q04844 or a polypeptide comprising an amino acid sequence having at least 95% sequence identity thereto).
   1682. The method of embodiment 748, wherein the disease or condition is Congenital Myasthenic Syndrome and/or wherein the heterologous nucleic acid sequence encodes CHRNA1 (e.g., a polypeptide represented by UniProt Accession number P02708 or a polypeptide comprising an amino acid sequence having at least 95% sequence identity thereto).
   1683. The method of embodiment 748, wherein the disease or condition is Congenital Myasthenic Syndrome and/or wherein the heterologous nucleic acid sequence encodes DOK7 (e.g., a polypeptide represented by UniProt Accession number Q18PE1 or a polypeptide comprising an amino acid sequence having at least 95% sequence identity thereto).
   1684. The method of embodiment 748, wherein the disease or condition is Congenital Myasthenic Syndrome and/or wherein the heterologous nucleic acid sequence encodes CHAT (e.g., a polypeptide represented by UniProt Accession number P28329 or a polypeptide comprising an amino acid sequence having at least 95% sequence identity thereto).
   1685. The method of embodiment 748, wherein the disease or condition is Congenital Myopathy and/or wherein the heterologous nucleic acid sequence encodes ACTA1 (e.g., a polypeptide represented by UniProt Accession number P68133 or a polypeptide comprising an amino acid sequence having at least 95% sequence identity thereto).
   1686. The method of embodiment 748, wherein the disease or condition is Congenital Myopathy and/or wherein the heterologous nucleic acid sequence encodes STAC3 (e.g., a polypeptide represented by UniProt Accession number Q96MF2 or a polypeptide comprising an amino acid sequence having at least 95% sequence identity thereto).
   1687. The method of embodiment 748, wherein the disease or condition is Congenital Myopathy and/or wherein the heterologous nucleic acid sequence encodes TPM3 (e.g., a polypeptide represented by UniProt Accession number P06753 or a polypeptide comprising an amino acid sequence having at least 95% sequence identity thereto).
   1688. The method of embodiment 748, wherein the disease or condition is Congenital Myotonic Dystrophy and/or wherein the heterologous nucleic acid sequence encodes DMPK (e.g., a polypeptide represented by UniProt Accession number Q09013 or a polypeptide comprising an amino acid sequence having at least 95% sequence identity thereto).
   1689. The method of embodiment 748, wherein the disease or condition is Cori Disease (Debrancher Enzyme Deficiency or Forbes Disease) and/or wherein the heterologous nucleic acid sequence encodes AGL (e.g., a polypeptide represented by UniProt Accession number P35573 or a polypeptide comprising an amino acid sequence having at least 95% sequence identity thereto).
   1690. The method of embodiment 748, wherein the disease or condition is Danon disease and/or wherein the heterologous nucleic acid sequence encodes LAMP2 (e.g., a polypeptide represented by UniProt Accession number P13473 or a polypeptide comprising an amino acid sequence having at least 95% sequence identity thereto).
   1691. The method of embodiment 748, wherein the disease or condition is Dejerine-Sottas Disease and/or wherein the heterologous nucleic acid sequence encodes MPZ (e.g., a polypeptide represented by UniProt Accession number P25189 or a polypeptide comprising an amino acid sequence having at least 95% sequence identity thereto).
   1692. The method of embodiment 748, wherein the disease or condition is Dejerine-Sottas Disease and/or wherein the heterologous nucleic acid sequence encodes EGR2 (e.g., a polypeptide represented by UniProt Accession number P11161 or a polypeptide comprising an amino acid sequence having at least 95% sequence identity thereto).
   1693. The method of embodiment 748, wherein the disease or condition is Dejerine-Sottas Disease and/or wherein the heterologous nucleic acid sequence encodes PMP22 (e.g., a polypeptide represented by UniProt Accession number Q01453 or a polypeptide comprising an amino acid sequence having at least 95% sequence identity thereto).
   1694. The method of embodiment 748, wherein the disease or condition is Dejerine-Sottas Disease and/or wherein the heterologous nucleic acid sequence encodes PRX (e.g., a polypeptide represented by UniProt Accession number Q9BXMO or a polypeptide comprising an amino acid sequence having at least 95% sequence identity thereto).
   1695. The method of embodiment 748, wherein the disease or condition is Distal Muscular Dystrophy, Welander and/or wherein the heterologous nucleic acid sequence encodes TIA1 (e.g., a polypeptide represented by UniProt Accession number P31483 or a polypeptide comprising an amino acid sequence having at least 95% sequence identity thereto).
   1696. The method of embodiment 748, wherein the disease or condition is Distal Muscular Dystrophy, Miyoshi and/or wherein the heterologous nucleic acid sequence encodes DYSF (e.g., a polypeptide represented by UniProt Accession number 075923 or a polypeptide comprising an amino acid sequence having at least 95% sequence identity thereto).
   1697. The method of embodiment 748, wherein the disease or condition is Distal myopathy with anterior tibial onset and/or wherein the heterologous nucleic acid sequence encodes DYSF (e.g., a polypeptide represented by UniProt Accession number 075923 or a polypeptide comprising an amino acid sequence having at least 95% sequence identity thereto).
   1698. The method of embodiment 748, wherein the disease or condition is Duchenne Muscular Dystrophy and/or wherein the heterologous nucleic acid sequence encodes Dystrophin (DMD) (e.g., a polypeptide represented by UniProt Accession number P11532 or a polypeptide comprising an amino acid sequence having at least 95% sequence identity thereto).
   1699. The method of embodiment 748, wherein the disease or condition is Duchenne Muscular Dystrophy and/or wherein the heterologous nucleic acid sequence encodes GALGT2, B4GALNT2 (e.g., a polypeptide represented by UniProt Accession number Q8NHYO or a polypeptide comprising an amino acid sequence having at least 95% sequence identity thereto).
   1700. The method of embodiment 748, wherein the disease or condition is Dysferlinopathy and/or wherein the heterologous nucleic acid sequence encodes Dysferlin (DYSF) (e.g., a polypeptide represented by UniProt Accession number 075923 or a polypeptide comprising an amino acid sequence having at least 95% sequence identity thereto).
   1701. The method of embodiment 748, wherein the disease or condition is Emery-Dreifuss Muscular Dystrophy and/or wherein the heterologous nucleic acid sequence encodes EMD (e.g., a polypeptide represented by UniProt Accession number P50402 or a polypeptide comprising an amino acid sequence having at least 95% sequence identity thereto).
   1702. The method of embodiment 748, wherein the disease or condition is Emery-Dreifuss Muscular Dystrophy and/or wherein the heterologous nucleic acid sequence encodes SYNE1 (e.g., a polypeptide represented by UniProt Accession number Q8NF91 or a polypeptide comprising an amino acid sequence having at least 95% sequence identity thereto).
   1703. The method of embodiment 748, wherein the disease or condition is Emery-Dreifuss Muscular Dystrophy and/or wherein the heterologous nucleic acid sequence encodes SYNE2 (e.g., a polypeptide represented by UniProt Accession number Q8WXHO or a polypeptide comprising an amino acid sequence having at least 95% sequence identity thereto).
   1704. The method of embodiment 748, wherein the disease or condition is Emery-Dreifuss Muscular Dystrophy and/or wherein the heterologous nucleic acid sequence encodes TMEM43 (e.g., a polypeptide represented by UniProt Accession number Q9BTV4 or a polypeptide comprising an amino acid sequence having at least 95% sequence identity thereto).
   1705. The method of embodiment 748, wherein the disease or condition is Emery-Dreifuss Muscular Dystrophy and/or wherein the heterologous nucleic acid sequence encodes LMNA (e.g., a polypeptide represented by UniProt Accession number P02545 or a polypeptide comprising an amino acid sequence having at least 95% sequence identity thereto).
   1706. The method of embodiment 748, wherein the disease or condition is Eulenberg Disease (Paramyotonia Congenita) and/or wherein the heterologous nucleic acid sequence encodes SCN4A (e.g., a polypeptide represented by UniProt Accession number P35499 or a polypeptide comprising an amino acid sequence having at least 95% sequence identity thereto).
   1707. The method of embodiment 748, wherein the disease or condition is Facioscapulohumeral Muscular Dystrophy and/or wherein the heterologous nucleic acid sequence encodes SMCHD1 (e.g., a polypeptide represented by UniProt Accession number A6NHR9 or a polypeptide comprising an amino acid sequence having at least 95% sequence identity thereto).
   1708. The method of embodiment 748, wherein the disease or condition is Facioscapulohumeral Muscular Dystrophy and/or wherein the heterologous nucleic acid sequence encodes LRIF1 (e.g., a polypeptide represented by UniProt Accession number Q5T3J3 or a polypeptide comprising an amino acid sequence having at least 95% sequence identity thereto).
   1709. The method of embodiment 748, wherein the disease or condition is Friedreich's ataxia and/or wherein the heterologous nucleic acid sequence encodes Frataxin (FXN) (e.g., a polypeptide represented by UniProt Accession number Q16595 or a polypeptide comprising an amino acid sequence having at least 95% sequence identity thereto).
   1710. The method of embodiment 748, wherein the disease or condition is Fukuyama Congenital Muscular Dystrophy and/or wherein the heterologous nucleic acid sequence encodes FKTN (e.g., a polypeptide represented by UniProt Accession number 075072 or a polypeptide comprising an amino acid sequence having at least 95% sequence identity thereto).
   1711. The method of embodiment 748, wherein the disease or condition is Glycogen storage disease II (Pompe disease or Acid Maltase Deficiency) and/or wherein the heterologous nucleic acid sequence encodes GAA (e.g., a polypeptide represented by UniProt Accession number P10253 or a polypeptide comprising an amino acid sequence having at least 95% sequence identity thereto).
   1712. The method of embodiment 748, wherein the disease or condition is Glycogenosis Type 10 (Glycogen storage disease X) and/or wherein the heterologous nucleic acid sequence encodes PGAM2 (e.g., a polypeptide represented by UniProt Accession number P15259 or a polypeptide comprising an amino acid sequence having at least 95% sequence identity thereto).
   1713. The method of embodiment 748, wherein the disease or condition is Glycogenosis Type 11 (Glycogen storage disease XI) and/or wherein the heterologous nucleic acid sequence encodes LDHA (e.g., a polypeptide represented by UniProt Accession number P00338 or a polypeptide comprising an amino acid sequence having at least 95% sequence identity thereto).
   1714. The method of embodiment 748, wherein the disease or condition is Glycogenosis Type 2 (Glycogen storage disease II or Pompe Disease or Acid maltase deficiency) and/or wherein the heterologous nucleic acid sequence encodes GAA (e.g., a polypeptide represented by UniProt Accession number P10253 or a polypeptide comprising an amino acid sequence having at least 95% sequence identity thereto).
   1715. The method of embodiment 748, wherein the disease or condition is Glycogenosis Type 3 (Glycogen storage disease Ill) and/or wherein the heterologous nucleic acid sequence encodes AGL (e.g., a polypeptide represented by UniProt Accession number P35573 or a polypeptide comprising an amino acid sequence having at least 95% sequence identity thereto).
   1716. The method of embodiment 748, wherein the disease or condition is Glycogenosis Type 5 (Glycogen storage disease V or McArdle Disease or Myophosphorylase Deficiency) and/or wherein the heterologous nucleic acid sequence encodes PYGM (e.g., a polypeptide represented by UniProt Accession number P11217 or a polypeptide comprising an amino acid sequence having at least 95% sequence identity thereto).
   1717. The method of embodiment 748, wherein the disease or condition is Glycogenosis Type 7 (Glycogen storage disease VII or Phosphofructokinase Deficiency or Tarui Disease) and/or wherein the heterologous nucleic acid sequence encodes PFKM (e.g., a polypeptide represented by UniProt Accession number P08237 or a polypeptide comprising an amino acid sequence having at least 95% sequence identity thereto).
   1718. The method of embodiment 748, wherein the disease or condition is Glycogenosis Type 9 (Glycogen storage disease IX) and/or wherein the heterologous nucleic acid sequence encodes PHKB (e.g., a polypeptide represented by UniProt Accession number Q93100 or a polypeptide comprising an amino acid sequence having at least 95% sequence identity thereto).
   1719. The method of embodiment 748, wherein the disease or condition is Glycogenosis Type 9 (Glycogen storage disease IX) and/or wherein the heterologous nucleic acid sequence encodes PHKA2 (e.g., a polypeptide represented by UniProt Accession number P46019 or a polypeptide comprising an amino acid sequence having at least 95% sequence identity thereto).
   1720. The method of embodiment 748, wherein the disease or condition is Hereditary Inclusion-Body Myositis and/or wherein the heterologous nucleic acid sequence encodes GNE (e.g., a polypeptide represented by UniProt Accession number Q9Y223 or a polypeptide comprising an amino acid sequence having at least 95% sequence identity thereto).
   1721. The method of embodiment 748, wherein the disease or condition is Integrin-Deficient Congenital Muscular Dystrophy and/or wherein the heterologous nucleic acid sequence encodes ITGA7 (e.g., a polypeptide represented by UniProt Accession number Q13683 or a polypeptide comprising an amino acid sequence having at least 95% sequence identity thereto).
   1722. The method of embodiment 748, wherein the disease or condition is Kennedy Disease (Spinal-Bulbar Muscular Atrophy) and/or wherein the heterologous nucleic acid sequence encodes Androgen receptor (AR) (e.g., a polypeptide represented by UniProt Accession number P10275 or a polypeptide comprising an amino acid sequence having at least 95% sequence identity thereto).
   1723. The method of embodiment 748, wherein the disease or condition is Kugelberg-Welander Disease and/or wherein the heterologous nucleic acid sequence encodes SMN1 (e.g., a polypeptide represented by UniProt Accession number Q16637 or a polypeptide comprising an amino acid sequence having at least 95% sequence identity thereto).
   1724. The method of embodiment 748, wherein the disease or condition is Lactate dehydrogenase A deficiency and/or wherein the heterologous nucleic acid sequence encodes LDHA (e.g., a polypeptide represented by UniProt Accession number P00338 or a polypeptide comprising an amino acid sequence having at least 95% sequence identity thereto).
   1725. The method of embodiment 748, wherein the disease or condition is Lactate Dehydrogenase B Deficiency and/or wherein the heterologous nucleic acid sequence encodes LDHB (e.g., a polypeptide represented by UniProt Accession number P07195 or a polypeptide comprising an amino acid sequence having at least 95% sequence identity thereto).
   1726. The method of embodiment 748, wherein the disease or condition is Lambert-Eaton Myasthenic Syndrome and/or wherein the heterologous nucleic acid sequence encodes CACNB2 (e.g., a polypeptide represented by UniProt Accession number Q08289 or a polypeptide comprising an amino acid sequence having at least 95% sequence identity thereto).
   1727. The method of embodiment 748, wherein the disease or condition is Laing Distal Myopathy and/or wherein the heterologous nucleic acid sequence encodes MYH7 (e.g., a polypeptide represented by UniProt Accession number A7E2Y1 or a polypeptide comprising an amino acid sequence having at least 95% sequence identity thereto).
   1728. The method of embodiment 748, wherein the disease or condition is Limb Girdle Muscular Dystrophy Type 2C (LGMD-2C) and/or wherein the heterologous nucleic acid sequence encodes Gamma-sarcoglycan (e.g., a polypeptide represented by UniProt Accession number Q13326 or a polypeptide comprising an amino acid sequence having at least 95% sequence identity thereto).
   1729. The method of embodiment 748, wherein the disease or condition is Limb Girdle Muscular Dystrophy Type 2D (LGMD-2D) and/or wherein the heterologous nucleic acid sequence encodes Alpha-sarcoglycan (e.g., a polypeptide represented by UniProt Accession number Q16586 or a polypeptide comprising an amino acid sequence having at least 95% sequence identity thereto).
   1730. The method of embodiment 748, wherein the disease or condition is Limb Girdle Muscular Dystrophy Type2E (LGMD-2E) and/or wherein the heterologous nucleic acid sequence encodes Beta-sarcoglycan (e.g., a polypeptide represented by UniProt Accession number Q16585 or a polypeptide comprising an amino acid sequence having at least 95% sequence identity thereto).
   1731. The method of embodiment 748, wherein the disease or condition is Limb Girdle Muscular Dystrophy Type 2F (LGMD-2F) and/or wherein the heterologous nucleic acid sequence encodes Delta-sarcoglycan (e.g., a polypeptide represented by UniProt Accession number Q92629 or a polypeptide comprising an amino acid sequence having at least 95% sequence identity thereto).
   1732. The method of embodiment 748, wherein the disease or condition is Merosin-Deficient Congenital Muscular Dystrophy and/or wherein the heterologous nucleic acid sequence encodes LAMA2 (e.g., a polypeptide represented by UniProt Accession number P24043 or a polypeptide comprising an amino acid sequence having at least 95% sequence identity thereto).
   1733. The method of embodiment 748, wherein the disease or condition is Muscle-Eye-Brain Disease and/or wherein the heterologous nucleic acid sequence encodes POMGNT1 (e.g., a polypeptide represented by UniProt Accession number Q8WZA1 or a polypeptide comprising an amino acid sequence having at least 95% sequence identity thereto).
   1734. The method of embodiment 748, wherein the disease or condition is Mitochondrial Myopathy and/or wherein the heterologous nucleic acid sequence encodes CHCHD10 (e.g., a polypeptide represented by UniProt Accession number Q8WYQ3 or a polypeptide comprising an amino acid sequence having at least 95% sequence identity thereto).
   1735. The method of embodiment 748, wherein the disease or condition is Miyoshi myopathy and/or wherein the heterologous nucleic acid sequence encodes DYSF (e.g., a polypeptide represented by UniProt Accession number 075923 or a polypeptide comprising an amino acid sequence having at least 95% sequence identity thereto).
   1736. The method of embodiment 748, wherein the disease or condition is Myoadenylate Deaminase Deficiency and/or wherein the heterologous nucleic acid sequence encodes AMPD1 (e.g., a polypeptide represented by UniProt Accession number P23109 or a polypeptide comprising an amino acid sequence having at least 95% sequence identity thereto).
   1737. The method of embodiment 748, wherein the disease or condition is Myofibrillar Myopathy 1 and/or wherein the heterologous nucleic acid sequence encodes DES (e.g., a polypeptide represented by UniProt Accession number P17661 or a polypeptide comprising an amino acid sequence having at least 95% sequence identity thereto).
   1738. The method of embodiment 748, wherein the disease or condition is Myofibrillar Myopathy 2 and/or wherein the heterologous nucleic acid sequence encodes CRYAB (e.g., a polypeptide represented by UniProt Accession number P02511 or a polypeptide comprising an amino acid sequence having at least 95% sequence identity thereto).
   1739. The method of embodiment 748, wherein the disease or condition is Myofibrillar Myopathy 3 and/or wherein the heterologous nucleic acid sequence encodes MYOT (e.g., a polypeptide represented by UniProt Accession number Q9UBF9 or a polypeptide comprising an amino acid sequence having at least 95% sequence identity thereto).
   1740. The method of embodiment 748, wherein the disease or condition is Myofibrillar Myopathy 4 (ZASP related myopathy) and/or wherein the heterologous nucleic acid sequence encodes LDB3, ZASP (e.g., a polypeptide represented by UniProt Accession number 075112 or a polypeptide comprising an amino acid sequence having at least 95% sequence identity thereto).
   1741. The method of embodiment 748, wherein the disease or condition is Myofibrillar Myopathy 5 and/or wherein the heterologous nucleic acid sequence encodes FLNC (e.g., a polypeptide represented by UniProt Accession number Q14315 or a polypeptide comprising an amino acid sequence having at least 95% sequence identity thereto).
   1742. The method of embodiment 748, wherein the disease or condition is Myofibrillar Myopathy 6 and/or wherein the heterologous nucleic acid sequence encodes BAG3 (e.g., a polypeptide represented by UniProt Accession number 095817 or a polypeptide comprising an amino acid sequence having at least 95% sequence identity thereto).
   1743. The method of embodiment 748, wherein the disease or condition is Myofibrillar Myopathy 7 and/or wherein the heterologous nucleic acid sequence encodes KY (e.g., a polypeptide represented by UniProt Accession number Q8NBH2 or a polypeptide comprising an amino acid sequence having at least 95% sequence identity thereto).
   1744. The method of embodiment 748, wherein the disease or condition is Myofibrillar Myopathy 8 and/or wherein the heterologous nucleic acid sequence encodes PYROXD1 (e.g., a polypeptide represented by UniProt Accession number Q8WU10 or a polypeptide comprising an amino acid sequence having at least 95% sequence identity thereto).
   1745. The method of embodiment 748, wherein the disease or condition is Myofibrillar Myopathy 9 and/or wherein the heterologous nucleic acid sequence encodes TTN (e.g., a polypeptide represented by UniProt Accession number Q8WZ42 or a polypeptide comprising an amino acid sequence having at least 95% sequence identity thereto).
   1746. The method of embodiment 748, wherein the disease or condition is Myofibrillar Myopathy 10 and/or wherein the heterologous nucleic acid sequence encodes SVIL (e.g., a polypeptide represented by UniProt Accession number 095425 or a polypeptide comprising an amino acid sequence having at least 95% sequence identity thereto).
   1747. The method of embodiment 748, wherein the disease or condition is Myofibrillar Myopathy 11 and/or wherein the heterologous nucleic acid sequence encodes UNC45B (e.g., a polypeptide represented by UniProt Accession number Q81WX7 or a polypeptide comprising an amino acid sequence having at least 95% sequence identity thereto).
   1748. The method of embodiment 748, wherein the disease or condition is Myofibrillar Myopathy 12 and/or wherein the heterologous nucleic acid sequence encodes MYL2 (e.g., a polypeptide represented by UniProt Accession number Q99972 or a polypeptide comprising an amino acid sequence having at least 95% sequence identity thereto).
   1749. The method of embodiment 748, wherein the disease or condition is Myotonic dystrophy Type 1 (Steinert Disease) and/or wherein the heterologous nucleic acid sequence encodes Myotonin-protein kinase (DMPK) (e.g., a polypeptide represented by UniProt Accession number Q09013 or a polypeptide comprising an amino acid sequence having at least 95% sequence identity thereto).
   1750. The method of embodiment 748, wherein the disease or condition is Myotonic dystrophy Type 2 and/or wherein the heterologous nucleic acid sequence encodes CNBP (e.g., a polypeptide represented by UniProt Accession number P62633 or a polypeptide comprising an amino acid sequence having at least 95% sequence identity thereto).
   1751. The method of embodiment 748, wherein the disease or condition is Myotubular Myopathy and/or wherein the heterologous nucleic acid sequence encodes MTM1 (e.g., a polypeptide represented by UniProt Accession number Q13496 or a polypeptide comprising an amino acid sequence having at least 95% sequence identity thereto).
   1752. The method of embodiment 748, wherein the disease or condition is Nemaline Myopathy 1 and/or wherein the heterologous nucleic acid sequence encodes TPM3 (e.g., a polypeptide represented by UniProt Accession number P06753 or a polypeptide comprising an amino acid sequence having at least 95% sequence identity thereto).
   1753. The method of embodiment 748, wherein the disease or condition is Nemaline Myopathy 2 and/or wherein the heterologous nucleic acid sequence encodes NEB (e.g., a polypeptide represented by UniProt Accession number P20929 or a polypeptide comprising an amino acid sequence having at least 95% sequence identity thereto).
   1754. The method of embodiment 748, wherein the disease or condition is Nemaline Myopathy 5A, 5B, 5C and/or wherein the heterologous nucleic acid sequence encodes TNNT1 (e.g., a polypeptide represented by UniProt Accession number P13805 or a polypeptide comprising an amino acid sequence having at least 95% sequence identity thereto).
   1755. The method of embodiment 748, wherein the disease or condition is Nemaline Myopathy 3 and/or wherein the heterologous nucleic acid sequence encodes ACTA1 (e.g., a polypeptide represented by UniProt Accession number P68133 or a polypeptide comprising an amino acid sequence having at least 95% sequence identity thereto).
   1756. The method of embodiment 748, wherein the disease or condition is Nemaline Myopathy 6 and/or wherein the heterologous nucleic acid sequence encodes KBTBD13 (e.g., a polypeptide represented by UniProt Accession number C9JR72 or a polypeptide comprising an amino acid sequence having at least 95% sequence identity thereto).
   1757. The method of embodiment 748, wherein the disease or condition is Nemaline Myopathy 4 and/or wherein the heterologous nucleic acid sequence encodes TPM2 (e.g., a polypeptide represented by UniProt Accession number P07951 or a polypeptide comprising an amino acid sequence having at least 95% sequence identity thereto).
   1758. The method of embodiment 748, wherein the disease or condition is Nemaline Myopathy 7 and/or wherein the heterologous nucleic acid sequence encodes CFL2 (e.g., a polypeptide represented by UniProt Accession number Q9Y281 or a polypeptide comprising an amino acid sequence having at least 95% sequence identity thereto).
   1759. The method of embodiment 748, wherein the disease or condition is Nemaline Myopathy 8 and/or wherein the heterologous nucleic acid sequence encodes KLHL40 (e.g., a polypeptide represented by UniProt Accession number Q2TBAO or a polypeptide comprising an amino acid sequence having at least 95% sequence identity thereto).
   1760. The method of embodiment 748, wherein the disease or condition is Nemaline Myopathy 9 and/or wherein the heterologous nucleic acid sequence encodes KLHL41 (e.g., a polypeptide represented by UniProt Accession number 060662 or a polypeptide comprising an amino acid sequence having at least 95% sequence identity thereto).
   1761. The method of embodiment 748, wherein the disease or condition is Nemaline Myopathy 10 and/or wherein the heterologous nucleic acid sequence encodes LMOD3 (e.g., a polypeptide represented by UniProt Accession number Q0VAK6 or a polypeptide comprising an amino acid sequence having at least 95% sequence identity thereto).
   1762. The method of embodiment 748, wherein the disease or condition is Nonaka Distal Myopathy and/or wherein the heterologous nucleic acid sequence encodes GNE (e.g., a polypeptide represented by UniProt Accession number Q9Y223 or a polypeptide comprising an amino acid sequence having at least 95% sequence identity thereto).
   1763. The method of embodiment 748, wherein the disease or condition is Oculopharynggeal muscular dystrophy and/or wherein the heterologous nucleic acid sequence encodes PABPN1 (e.g., a polypeptide represented by UniProt Accession number Q86U42 or a polypeptide comprising an amino acid sequence having at least 95% sequence identity thereto).
   1764. The method of embodiment 748, wherein the disease or condition is Omithine Transcarbamylase deficiency and/or wherein the heterologous nucleic acid sequence encodes OTC (e.g., a polypeptide represented by UniProt Accession number P00480 or a polypeptide comprising an amino acid sequence having at least 95% sequence identity thereto).
   1765. The method of embodiment 748, wherein the disease or condition is Paramyotonia Congenita and/or wherein the heterologous nucleic acid sequence encodes SCN4A (e.g., a polypeptide represented by UniProt Accession number P35499 or a polypeptide comprising an amino acid sequence having at least 95% sequence identity thereto).
   1766. The method of embodiment 748, wherein the disease or condition is Periodic Paralysis, hypokalemic and/or wherein the heterologous nucleic acid sequence encodes CACNA1S (e.g., a polypeptide represented by UniProt Accession number Q13698 or a polypeptide comprising an amino acid sequence having at least 95% sequence identity thereto).
   1767. The method of embodiment 748, wherein the disease or condition is Periodic Paralysis, hyperkalemic and/or wherein the heterologous nucleic acid sequence encodes SCN4A (e.g., a polypeptide represented by UniProt Accession number P35499 or a polypeptide comprising an amino acid sequence having at least 95% sequence identity thereto).
   1768. The method of embodiment 748, wherein the disease or condition is Phosphoglycerate Kinase Deficiency and/or wherein the heterologous nucleic acid sequence encodes PGK1 (e.g., a polypeptide represented by UniProt Accession number P00558 or a polypeptide comprising an amino acid sequence having at least 95% sequence identity thereto).
   1769. The method of embodiment 748, wherein the disease or condition is Polymyositis and/or wherein the heterologous nucleic acid sequence encodes PMSCL2 (e.g., a polypeptide represented by UniProt Accession number Q01780 or a polypeptide comprising an amino acid sequence having at least 95% sequence identity thereto).
   1770. The method of embodiment 748, wherein the disease or condition is Polymyositis and/or wherein the heterologous nucleic acid sequence encodes PMSCL1 (e.g., a polypeptide represented by UniProt Accession number Q06265 or a polypeptide comprising an amino acid sequence having at least 95% sequence identity thereto).
   1771. The method of embodiment 748, wherein the disease or condition is Progressive External Ophthalmoplegia and/or wherein the heterologous nucleic acid sequence encodes POLG (e.g., a polypeptide represented by UniProt Accession number P54098 or a polypeptide comprising an amino acid sequence having at least 95% sequence identity thereto).
   1772. The method of embodiment 748, wherein the disease or condition is Progressive External Ophthalmoplegia and/or wherein the heterologous nucleic acid sequence encodes POLG2 (e.g., a polypeptide represented by UniProt Accession number Q9UHN1 or a polypeptide comprising an amino acid sequence having at least 95% sequence identity thereto).
   1773. The method of embodiment 748, wherein the disease or condition is Progressive External Ophthalmoplegia and/or wherein the heterologous nucleic acid sequence encodes SLC25A4 (e.g., a polypeptide represented by UniProt Accession number P12235 or a polypeptide comprising an amino acid sequence having at least 95% sequence identity thereto).
   1774. The method of embodiment 748, wherein the disease or condition is Progressive External Ophthalmoplegia and/or wherein the heterologous nucleic acid sequence encodes TWNK (e.g., a polypeptide represented by UniProt Accession number Q96RR1 or a polypeptide comprising an amino acid sequence having at least 95% sequence identity thereto).
   1775. The method of embodiment 748, wherein the disease or condition is Spinal Muscular Atrophy type 3—Kugelberg-Welander Disease and/or wherein the heterologous nucleic acid sequence encodes Survival motor neuron protein (SMN), SMN1 (e.g., a polypeptide represented by UniProt Accession number Q16637 or a polypeptide comprising an amino acid sequence having at least 95% sequence identity thereto).
   1776. The method of embodiment 748, wherein the disease or condition is Thomsen Disease (Myotonia Congenita (autosomal dominant)) and/or wherein the heterologous nucleic acid sequence encodes CLCN1 (e.g., a polypeptide represented by UniProt Accession number P35523 or a polypeptide comprising an amino acid sequence having at least 95% sequence identity thereto).
   1777. The method of embodiment 748, wherein the disease or condition is Walker-Warburg Syndrome and/or wherein the heterologous nucleic acid sequence encodes POMT1 (e.g., a polypeptide represented by UniProt Accession number Q9Y6A1 or a polypeptide comprising an amino acid sequence having at least 95% sequence identity thereto).
   1778. The method of embodiment 748, wherein the disease or condition is X-linked myotubular myopathy and/or wherein the heterologous nucleic acid sequence encodes Myotubularin (MTM1) (e.g., a polypeptide represented by UniProt Accession number Q13496 or a polypeptide comprising an amino acid sequence having at least 95% sequence identity thereto).
   1779. The method of embodiment 748, wherein the disease or condition is Werdnig-Hoffmann Disease—Spinal Muscular Atrophy type 1 and/or wherein the heterologous nucleic acid sequence encodes SMN1 (e.g., a polypeptide represented by UniProt Accession number Q16637 or a polypeptide comprising an amino acid sequence having at least 95% sequence identity thereto).
   1780. The method of embodiment 748, wherein the disease or condition is Osteogenesis imperfecta (brittle bone disease)Type I, II, Ill, or IV and/or wherein the heterologous nucleic acid sequence encodes COL1A1 (e.g., a polypeptide represented by UniProt Accession number P02452 or a polypeptide comprising an amino acid sequence having at least 95% sequence identity thereto).
   1781. The method of embodiment 748, wherein the disease or condition is Osteogenesis imperfecta (brittle bone disease)Type I, II, III, or IV and/or wherein the heterologous nucleic acid sequence encodes COL1A2 (e.g., a polypeptide represented by UniProt Accession number P08123 or a polypeptide comprising an amino acid sequence having at least 95% sequence identity thereto).
   1782. The method of embodiment 748, wherein the disease or condition is Hereditary angioedema and/or wherein the heterologous nucleic acid sequence encodes Plasma protease C1 inhibitor (SERPING1, C1NH) (e.g., a polypeptide represented by UniProt Accession number P05155 or a polypeptide comprising an amino acid sequence having at least 95% sequence identity thereto).
   1783. The method of embodiment 748, wherein the disease or condition is Osteogenesis imperfecta Type V and/or wherein the heterologous nucleic acid sequence encodes Interferon-induced transmembrane protein 5 (IFITM5, IFM5) (e.g., a polypeptide represented by UniProt Accession number A6NNB3 or a polypeptide comprising an amino acid sequence having at least 95% sequence identity thereto).
   1784. The method of embodiment 748, wherein the disease or condition is Osteogenesis Imperfecta Type VI and/or wherein the heterologous nucleic acid sequence encodes Pigment epithelium-derived factor (SERPINF1, PEDF) (e.g., a polypeptide represented by UniProt Accession number P36955 or a polypeptide comprising an amino acid sequence having at least 95% sequence identity thereto).
   1785. The method of embodiment 748, wherein the disease or condition is Osteogenesis Imperfecta Type VII and/or wherein the heterologous nucleic acid sequence encodes Cartilage-associated protein (CRTAP) (e.g., a polypeptide represented by UniProt Accession number 075718 or a polypeptide comprising an amino acid sequence having at least 95% sequence identity thereto).
   1786. The method of embodiment 748, wherein the disease or condition is Osteogenesis Imperfecta Type VIII and/or wherein the heterologous nucleic acid sequence encodes Prolyl 3-hydroxylase 1 (P3H1, LEPRE1) (e.g., a polypeptide represented by UniProt Accession number Q32P28 or a polypeptide comprising an amino acid sequence having at least 95% sequence identity thereto).
   1787. The method of embodiment 748, wherein the disease or condition is Osteogenesis Imperfecta Type IX and/or wherein the heterologous nucleic acid sequence encodes PPIB (e.g., a polypeptide represented by UniProt Accession number P23284 or a polypeptide comprising an amino acid sequence having at least 95% sequence identity thereto).
   1788. The method of embodiment 748, wherein the disease or condition is Maple syrup urine disease and/or wherein the heterologous nucleic acid sequence encodes BCKDHA (e.g., a polypeptide represented by UniProt Accession number P12694 or a polypeptide comprising an amino acid sequence having at least 95% sequence identity thereto).
   1789. The method of embodiment 748, wherein the disease or condition is Maple syrup urine disease and/or wherein the heterologous nucleic acid sequence encodes BCKDHB (e.g., a polypeptide represented by UniProt Accession number P21953 or a polypeptide comprising an amino acid sequence having at least 95% sequence identity thereto).
   1790. The method of embodiment 748, wherein the disease or condition is Maple syrup urine disease and/or wherein the heterologous nucleic acid sequence encodes DBT (e.g., a polypeptide represented by UniProt Accession number P11182 or a polypeptide comprising an amino acid sequence having at least 95% sequence identity thereto).
   1791. The method of embodiment 748, wherein the disease or condition is Alpha-mannosidosis and/or wherein the heterologous nucleic acid sequence encodes Lysosomal alpha-mannosidase (MAN2B1) (e.g., a polypeptide represented by UniProt Accession number 000754 or a polypeptide comprising an amino acid sequence having at least 95% sequence identity thereto).
   1792. The method of embodiment 748, wherein the disease or condition is Beta-mannosidosis and/or wherein the heterologous nucleic acid sequence encodes Beta-mannosidase (MANBA) (e.g., a polypeptide represented by UniProt Accession number 000462 or a polypeptide comprising an amino acid sequence having at least 95% sequence identity thereto).
   1793. The method of embodiment 748, wherein the disease or condition is Glycogen storage disease la (Von Gierke disease) and/or wherein the heterologous nucleic acid sequence encodes Glucose-6-phosphatase catalytic subunit 1 (G6PC1) (e.g., a polypeptide represented by UniProt Accession number P35575 or a polypeptide comprising an amino acid sequence having at least 95% sequence identity thereto).
   1794. The method of embodiment 748, wherein the disease or condition is Bloch-Sulzberger Syndrome and/or wherein the heterologous nucleic acid sequence encodes IKBKG (e.g., a polypeptide represented by UniProt Accession number Q9Y6K9 or a polypeptide comprising an amino acid sequence having at least 95% sequence identity thereto).
   1795. The method of embodiment 748, wherein the disease or condition is Cholesterol Ester Storage Disease and/or wherein the heterologous nucleic acid sequence encodes LIPA (e.g., a polypeptide represented by UniProt Accession number P38571 or a polypeptide comprising an amino acid sequence having at least 95% sequence identity thereto).
   1796. The method of embodiment 748, wherein the disease or condition is Danon Disease and/or wherein the heterologous nucleic acid sequence encodes Lysosome-associated membrane glycoprotein 2 (LAMP2) (e.g., a polypeptide represented by UniProt Accession number P13473 or a polypeptide comprising an amino acid sequence having at least 95% sequence identity thereto).
   1797. The method of embodiment 748, wherein the disease or condition is Cystic Fibrosis and/or wherein the heterologous nucleic acid sequence encodes CTFR (e.g., a polypeptide represented by UniProt Accession number A4L9V0 or a polypeptide comprising an amino acid sequence having at least 95% sequence identity thereto).
   1798. The method of embodiment 748, wherein the disease or condition is Rheumatoid Arthritis and/or wherein the heterologous nucleic acid sequence encodes TNF (e.g., a polypeptide represented by UniProt Accession number P01375 or a polypeptide comprising an amino acid sequence having at least 95% sequence identity thereto).
   1799. The method of embodiment 748, wherein the disease or condition is Alpha-1 Antitrypsin Deficiency and/or wherein the heterologous nucleic acid sequence encodes Alpha-1-antitrypsin (Alpha1-AT), AAT, SERPINA1 (e.g., a polypeptide represented by UniProt Accession number P01009 or a polypeptide comprising an amino acid sequence having at least 95% sequence identity thereto).
   1800. The method of embodiment 748, wherein the disease or condition is Hemophilia A and/or wherein the heterologous nucleic acid sequence encodes Coagulation factor VIII (e.g., a polypeptide represented by UniProt Accession number P00451 or a polypeptide comprising an amino acid sequence having at least 95% sequence identity thereto).
   1801. The method of embodiment 748, wherein the disease or condition is Hemophilia B and/or wherein the heterologous nucleic acid sequence encodes Coagulation factor IX (e.g., a polypeptide represented by UniProt Accession number P00740 or a polypeptide comprising an amino acid sequence having at least 95% sequence identity thereto).
   1802. The method of embodiment 748, wherein the disease or condition is Homozygous Familial Hypercholesterolemia (FHCL1) and/or wherein the heterologous nucleic acid sequence encodes Low-density lipoprotein receptor (LDLR) (e.g., a polypeptide represented by UniProt Accession number P01130 or a polypeptide comprising an amino acid sequence having at least 95% sequence identity thereto).
   1803. The method of embodiment 748, wherein the disease or condition is Mucopolysaccharidosis Type VI and/or wherein the heterologous nucleic acid sequence encodes Arylsulfatase B (ARSB) (e.g., a polypeptide represented by UniProt Accession number P15848 or a polypeptide comprising an amino acid sequence having at least 95% sequence identity thereto).
   1804. The method of embodiment 748, wherein the disease or condition is Familial Lipoprotein Lipase Deficiency and/or wherein the heterologous nucleic acid sequence encodes LPL (e.g., a polypeptide represented by UniProt Accession number P06858 or a polypeptide comprising an amino acid sequence having at least 95% sequence identity thereto).
   1805. The method of embodiment 748, wherein the disease or condition is Fragile X syndrome and/or wherein the heterologous nucleic acid sequence encodes FMR1 (e.g., a polypeptide represented by UniProt Accession number Q06787 or a polypeptide comprising an amino acid sequence having at least 95% sequence identity thereto).
   1806. The method of any one of embodiments 748 to 1805, wherein the subject is a mammal, e.g., a human.
   1807. A cell, cell-free system, or other translation system, comprising the capsid polypeptide of any one of embodiments 1 to 531, the nucleic acid molecule of any one of embodiments 532 to 551, or virus particle of any one of embodiments 552 to 736.
   1808. A method of making a virus (e.g., a dependoparvovirus particle such as an adeno-associated virus (AAV) particle), comprising:
(a) providing a cell, cell-free system, or other translation system, comprising the nucleic acid of any one of embodiments 532 to 551; and
(b) cultivating the cell, cell-free system, or other translation system, under conditions suitable for the production of the virus particle,
(c) thereby making the virus particle.
   1809. The method of embodiment 1808, wherein the cell, cell-free system, or other translation system comprises a second nucleic acid molecule and at least a portion of said second nucleic acid molecule is packaged in the dependoparvovirus particle.
   1810. The method of embodiment 1809, wherein the second nucleic acid comprises a payload, e.g., a heterologous nucleic acid sequence encoding a therapeutic product, e.g., as described herein.
   1811. The method of any one of embodiments 1808 to 1810, wherein the nucleic acid molecule of any one of embodiments 532 to 551 mediates the production of a virus particle which does not include said nucleic acid of any one of embodiments 532 to 551 or fragment thereof.
   1812. The method of any one of embodiments 1808 to 1811, wherein the nucleic acid molecule of any one of embodiments 532 to 551 mediates the production of a virus particle at a level similar, or at least 10% greater than the production level mediated by a nucleic acid comprising SEQ ID NO:2 in an otherwise similar production system.
   1813. A composition, e.g., a pharmaceutical composition, comprising a virus particle of any one of embodiments 552 to 736 or a virus particle produced by the method of any one of embodiments 1808 to 1812, and a pharmaceutically acceptable carrier or excipient.
   1814. The capsid polypeptide of any one of embodiments 1 to 531, the nucleic acid molecule of any one of embodiments 532 to 551, the virus particle of any one of embodiments 552 to 736, or the composition of embodiment 1813, for use in treating a disease or condition in a subject, optionally wherein (a) the subject is as defined in embodiment 1323 and/or (b) the disease or condition is as defined in any one of embodiments 748 to 1805.
   1815. The capsid polypeptide of any one of embodiments 1 to 531, the nucleic acid molecule of any one of embodiments 532 to 551, or the virus particle of any one of embodiments 552 to 736, or the composition of embodiment 1813, for use in the manufacture of a medicament for use in treating a disease or condition in a subject, optionally wherein (a) the subject is as defined in embodiment 1323 and/or (b) the disease or condition is as defined in any one of embodiments 748 to 1805.
   1816. A host cell comprising the nucleic acid of any one of embodiments 532 to 551.
   1817. The host cell of embodiment 1816, wherein the host cell comprises a nucleic acid encoding a rep protein.
   1818. The host cell of embodiment 1816 or 1817, wherein the host cell comprises a nucleic acid encoding a cap protein.
   1819. The host cell of any one of embodiments 1816 to 1818, wherein the host cell comprises a nucleic acid comprising one or more helper sequences.
   1820. The host cell of any one of embodiments 1816 to 1819, wherein the host cell comprises a nucleic acid comprising a payload, e.g., a heterologous nucleic acid sequence encoding a therapeutic product, e.g., as described herein.
   1821. The host cell of any one of embodiments 1816 to 1820, which is a mammalian host cell.
   1822. The host cell of embodiment 1821, which is a human host cell.
   1823. The host cell of any one of embodiments 1816 to 1820, which is an insect host cell.
   1824. The host cell of any one of embodiments 1816 to 1823, wherein the host cell is selected from cell types described in Section 5.5.
   1825. The host cell of any one of embodiments 1816 to 1824, which is a packaging cell line.
   1826. The host cell of embodiment 1825, which is configured to package a virus (a) according to any one of embodiments 552 to 736 and/or (b) useful for performing a method according to any one of embodiments 743 to 1806.
   1827. The method of any one of embodiments 1808 to 1812, wherein the cell is a host cell according to any one of embodiments 1816 to 1826.

### 7. EXAMPLES

The invention is further illustrated by the following examples. The examples are provided for illustrative purposes only and are not to be construed as limiting the scope or content of the invention in any way.

Unless otherwise noted, rates of biodistribution, transduction and/or production are presented as fold-improvement over the rates exhibited by a virus particle comprising capsid polypeptides of SEQ ID NO:1. In some cases, rates are presented in log 2 notation.

### 7.1. EXAMPLE 1: HIGH THROUGHPUT AAV9 LIBRARY #1 EVALUATION IN THE CNS

### 7.1.1. Materials & Methods

### 7.1.1.1. Library #1 Creation

Using machine learning algorithms trained on data from hundreds of thousands of capsid variants across multiple serotypes, including AAV particle production efficiency, and transduction and biodistribution across multiple tissues, a library (“Library #1”) of 1E5 capsid variants of wild-type AAV9 was designed with the goals of producing a capsid that would package into AAV particles, transduce central nervous system tissues after intravenous injection with high efficiency, and de-target the liver and other tissue types. The designed capsid polypeptides were cloned into plasmids to create a library of plasmids encoding the capsid variants. A library of AAV variant genomes encoding each variant's capsid and a unique capsid variant barcode identifier was cloned into two ITR plasmid backbones as described previously (Ogden et al., 2019, Science 366 (6469):1139-1143; doi: 10.1126/science.aaw2900, hereby incorporated by reference in its entirety), one with expression of the capsid gene under the control of a human EF1-alpha promoter (hEF1-alpha) and the other with expression of the capsid gene under the control of a ubiquitous CBh promoter. Both hEF1-alpha and CBh are ubiquitous promoters are expressed in CNS and other tissues. Each plasmid backbone contained a unique genomic identifier (“backbone tag”) enabling analysis of biodistribution and transduction efficiencies via each of the two promoters. Each capsid polypeptide variant was included in combination with between 1-500 unique genomic identifiers (“barcodes”) to enable measurement of biological replicates for each virus comprising a unique capsid polypeptide. A library of AAV capsid variants, each comprising a genome encoding that variant's capsid polypeptides, was produced via transient triple transfection of adherent HEK293T. Transfections were completed at a 1:1:1/150 ratio of Helper, Rep, and VAR-inside-ITR plasmid, which has been optimized with these plasmids to limit cross packaging. Cells were harvested, lysed, and purified by a sequence of steps: (1) diafiltration, (2) tangential flow filtration (TFF), (3) iodixanol gradient purification, and (4) buffer exchange. The produced virus was tested for suitable sterility and endotoxin, and titer performed by ddPCR.

### 7.1.1.2. In Vitro Evaluation of Library #1

Data was prepared as described below. To measure each variant's packaging efficiency (or “production”), barcodes from vector genomes in the plasmid library and produced AAV library were prepared for Illumina sequencing using two rounds of PCR. Production efficiency for each of the produced AAV particle variants was normalized for its abundance in the input plasmid library, and was expressed by comparing barcode sequencing levels for each variant in the produced AAV particle vector pool to the barcode sequence levels for each variant in the input plasmid library used to create the AAV vector pool. The measurements of variant frequency in the AAV vector library also enable downstream normalization of biodistribution and transduction measurements by variant frequency in the input vector library.

### 7.1.1.3. In Vivo Evaluation of Library #1 in Non-Human Primate

All NHP experiments were conducted in accordance with institutional policies and NIH guidelines. Two female African Green Monkeys (Chlorocebus sabaeus) weighing 3.04 and 3.33 kg and seronegative for anti-AAV9 neutralizing antibodies (serum NAb titers <1:4 based on in vitro NAb assay) were selected for the study. Prior to test article administrations, samples of blood were collected. The animals were anesthetized with ketamine and xylazine and received an intravenous injection of a mixture of the vector libraries having the different promoters (doses: 8.5e13 vg/kg and 9.5e13 vg/kg). During the in-life period the animals were monitored for signs of inflammation and were treated with weekly IM injections of steroids (methylprednisolone, 8 mg/kg), and dexamethasone as needed according to the animal facility's SOPs and recommendations from the veterinarian. Serum samples were collected at 1 h, 4 h and 24 h, and weekly after the injections. The animals were sacrificed 4 weeks after the injections and tissues were collected for biodistribution and transduction analyses. The tissues collected are shown in Table 5. All samples were collected into RNAlater® (Sigma-Aldrich) and incubated overnight at 4° C., after which the RNAlater® was drained and samples were frozen at −80° C. or maintained at 4° C. in RNAlater® (small subset of samples). In addition, samples of serum and cerebrospinal fluid were collected at necropsy and stored at −80° C.

TABLE 5
List of Tissues Collected

Adrenal Gland
Artery (Carotid)
Aorta
Brain (Full Brain Except Cerebellum) Coronal Axis Slices
Cerebellum Sagittal Axis Slices
Dorsal Root Ganglion (Cervical)
Dorsal Root Ganglion (Thoracic)
Dorsal Root Ganglion (Lumbar)
Gonad (Ovaries)
Heart, Basal
Heart, Apex
Kidney
Liver
Lung
Lymph Nodes, Cervical
Neural Retinal
Sciatic Nerve
Skeletal Muscle, Gastrocnemius
Skeletal Muscle, Quadriceps
Spinal Cord (Cervical)
Spinal Cord (Thoracic)
Spinal Cord (Lumbar)
Spleen

Brain slices were dissected to isolate regions including, but not limited to, frontal cortex, temporal cortex, motor cortex, hippocampus, basal ganglia, midbrain, brainstem, and cerebellum. For all biodistribution and transduction analyses, total DNA and RNA was extracted from tissue samples with Trizol/chloroform and isopropanol precipitation. Reverse transcription was done with Protoscript II Reverse Transcriptase (NEB) utilizing primers that were specific to the vector transgene and included unique molecular identifiers (UMIs). Control reactions lacking the reverse transcriptase enzyme (-RT control) were also prepared. Quantification of biodistribution (based on viral DNA quantification) and transduction (based on viral transcript RNA quantification) was done with Luna Universal Probe qPCR Master Mix (NEB) using primers and probes specific to the transgene construct. Finally, samples were prepared for next-generation sequencing by amplifying the transgene barcode regions with primers compatible with Illumina NGS platform and sequenced with NextSeq 550 (Illumina).

After sequencing, the barcode tags were extracted from reads with the expected amplicon structure, and the abundance (number of reads or number of UMIs) of each barcode was recorded. Analyses were restricted to the set of barcodes that were present in the input plasmid sample, as measured by a separate sequencing assay that targeted the variant regions of the input plasmid sample.

To aggregate packaging replicates, the read counts from replicate virus production samples were summed. To aggregate biodistribution samples, read counts from samples from the same tissue were summed. To aggregate transduction samples, the number of transduction events (measured by unique id tags detected) from samples from the same tissue were summed.

Virus packaging and transduction of tissue were calculated using a Bayesian model with aggregated production and/or transduction samples as the input. Briefly, probabilistic programming and stochastic variational inference were used to model the measurement process and sources of decoupling (e.g., cross-packaging, template switching, and errors in DNA synthesis) between the actual test virus particles and their designed sequences, and to calculate virus production and transduction (in various tissue samples), and error rates. Biodistribution of tissue was calculated by normalizing aggregated biodistribution samples with input virus abundance. The output was the log 2-transformed mean of the calculated distribution or normalized rate relative to the wild-type (WT) AAV9. Thus, positive values indicate higher performance than WT for the measured property, and negative values indicate lower-than-WT performance. Unless otherwise noted, all results are reported from aggregated values from the library comprising the CBh promoter.

### 7.1.2. Results

One variant capsid (VAR-B1) was identified that showed a more than 75-fold increase in transduction and biodistribution across multiple brain regions relative to wild-type AAV9. This variant additionally packaged/produced AAV virus particles efficiently from the HEK293T cell production system. Property measurements for VAR-B1, averaged across all tissue pieces for the indicated tissue type and across both NHPs, are reported in Table 6. Assessment of off-target tissues showed that VAR-B1 exhibits reduced biodistribution to liver (approximately 2-fold lower than wild-type AAV9), reduced transduction of liver tissue (approximately 6-fold lower than AAV9), and reduced biodistribution to spleen, muscle and heart (approximately 4-fold lower, 2-fold lower and 2-fold lower, respectively, relative to wild-type AAV9), indicating that this AAV variant capsid is specific for the brain regions of interest after intravenous administration. In addition, relative transduction of dorsal root ganglia to overall brain transduction for VAR-B1 was substantially lower than for AAV9 (0.023 for VAR-B1 vs. 1.0 for wtAAV9), indicating a substantial (over 40-fold) specificity for brain regions of interest relative to the DRG. In addition, measurements of biodistribution and transduction for VAR-B1 were well-correlated across the two primates. Taken together, these findings indicate that capsid polypeptides such as VAR-B1 and as described herein are suitable for gene therapies where targeting the brain is important, for example, as described herein. Without being bound by theory, such gene therapies are preferred over existing alternatives, such as those using wild-type AAV9, due to their enhanced specificity for the brain regions of interest and substantial increase in transduction efficiency.

TABLE 6
Mean production, biodistribution and transduction of VAR-
B1. All values reported as log2 relative to wtAAV9. All
values are calculated from aggregating across all tissue
samples collected from both NHP animals in the study.

| | Log₂ relative to wtAAV9 |
|---|---|
| Property | (fold-change) |
| Production - HEK293T cells | −0.6675 (0.63) |
| Brain transduction | 6.2845 (77.95) |
| Brain biodistribution | 5.5945 (48.31) |
| Cerebellum transduction | 6.5867 (96.11) |
| Cerebellum biodistribution | 5.9750 (62.90) |
| Cortex transduction | 6.4616 (88.13) |
| Cortex biodistribution | 5.8256 (56.71) |
| Midbrain/Brainstem transduction | 5.9003 (59.72) |
| Midbrain/Brainstem biodistribution | 5.3450 (40.65) |
| Spinal cord transduction | 6.0535 (66.42) |
| Spinal cord biodistribution | 4.0654 (16.74) |
| Subcortex transduction | 6.5805 (95.70) |
| Subcortex biodistribution | 5.4570 (43.93) |
| Liver transduction | −2.6841 (0.16) |
| Liver biodistribution | −1.0338 (0.49) |
| Heart transduction | −0.9601 (0.51) |
| Heart biodistribution | −0.8876 (0.54) |
| Muscle transduction | 0.1441 (1.10) |
| Muscle biodistribution | −0.8026 (0.57) |
| DRG biodistribution | 0.8356 (1.78) |
| Spleen biodistribution | −2.0366 (0.24) |

### 7.2. EXAMPLE 2: LOW THROUGHPUT IN VIVO EVALUATION OF VAR-B1 IN NHP

In order to confirm the performance of virus particles comprising the capsid polypeptide of VAR-B1 and further investigate its properties, virus particle comprising the VAR-B1 capsid polypeptide and carrying a transgene encoding a fluorescent protein was synthesized and tested in in vivo and in vitro studies alongside virus particle comprising a wild-type AAV9 capsid polypeptide and different fluorescent protein, as described below.

### 7.2.1. Materials & Methods

### 7.2.1.1. Virus Design And Production

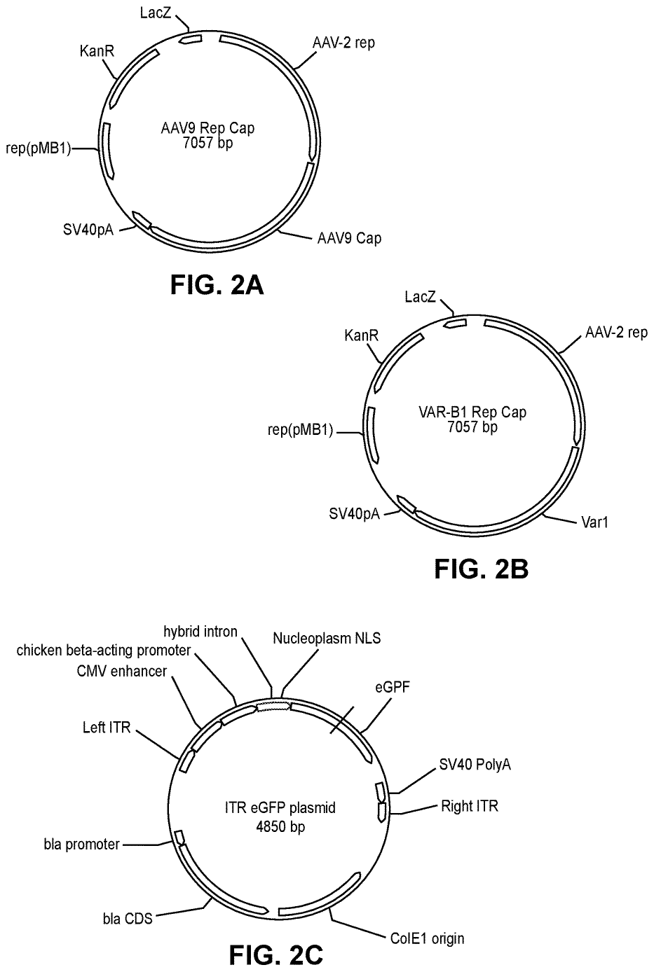

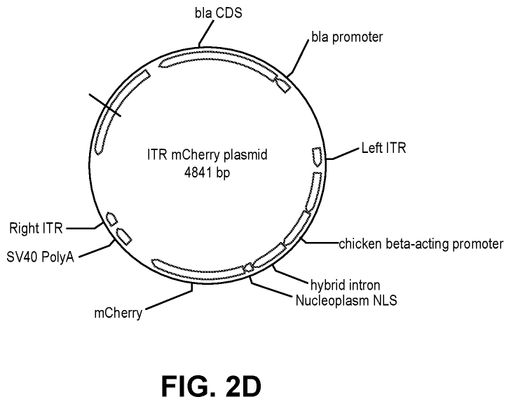

Virus particles comprising the VAR-B1 and wild-type AAV9 capsids were produced individually via transient triple transfection of adherent HEK293T cells (pRepCap(VAR-B1), pHELP (pALD X-80, Aldevron), pITR.Cbh.GFP; and pRepCap(wtAAV9), pHELP (pALD X-80, Aldevron), pITR.CBh.mCherry) (See FIGS. 2A-2D) followed by purification as described in Example 1. Each variant capsid was produced with a self-complementary (scAAV) genome containing 5 distinct regions: One, the ubiquitous CBh promoter (CMV enhancer, Cba promoter, CBA/MVM hybrid intron); two, a fluorescent reporter unique to each virus with a nuclear localization signal (NLS) tag (GFP in the case of VAR-B1 virus particles and mCherry in the case of wild-type AAV9 virus particles); three, a unique barcode. Each genome was produced with 8 unique barcodes per florescent reporter, which were included to provide technical replicates for each capsid within the single study. Four, the SV40 PolyA termination signal; and five, left and right ITRs capable of enabling self-complementary genome packaging in the virus particles (See FIGS. 2A-2D). After individual purification of each virus, concentrations and amounts were quantified using ddPCR and a final test article was formulated at 50% VAR-B1: 50% AAV9.

### 7.2.1.2. In Vivo Study Design

All NHP experiments were conducted in accordance with institutional policies and NIH guidelines. Two female Cynomolgus macaque NHPs weighing 3 kg and seronegative for anti-AAV9 neutralizing antibodies (serum NAb titers <1:4 based on in vitro NAb assay) were selected for the study. Animals were treated with Kenalog (10 mg/mL, 0.8 mg/kg) on Day −8 and 2x weekly for the duration of the study. Prior to test article administration, samples of blood were collected. The animals received an intravenous injection of final test article virus containing VAR-B1 and AAV9 (total combined doses: 9e12 vg/kg (low dose) and 1.9e13 vg/kg (high dose)). During the in-life period the animals were monitored according to the animal facility's SOPs. Serum samples were collected at 1 day, 2 days, 4 days, and weekly post injection. The animals were sacrificed 4 weeks after the injections, perfused with cold saline, and tissues were collected for biodistribution, transduction, and histology analyses. The tissues and collection methods are shown in Table 7. For brain, the left hemisphere was dissected, and flash froze in 4 mm slices (stored at −80° C.) and the right hemisphere was sliced at 4 mm and fixed in 10% naturally buffered formalin at room temperature for 48 hours before being moved to cold PBS. Other samples were collected and either flash frozen, formalin fixed, or collected into RNA/ater@(Sigma-Aldrich), as indicated in Table 7. Samples collected in RNAlater®were incubated overnight at 4° C., after which the RNA/ater @was drained and samples were frozen at −80° C. In addition, serum samples were collected at necropsy and stored at −80° C.

TABLE 7
List of Tissues Collected

| Tissue | Collection Method |
|---|---|
| Left brain coronal axis slices | Flash Frozen |
| Right brain coronal axis slices | Formalin Fixed |
| Brainstem | Flash Frozen, Formalin Fixed |
| dorsal root ganglion (cervical) | Flash Frozen, Formalin Fixed |
| dorsal root ganglion (thoracic) | Flash Frozen, Formalin Fixed |
| dorsal root ganglion (lumbar) | Flash Frozen, Formalin Fixed |
| dorsal root ganglion (sacral) | Flash Frozen, Formalin Fixed |
| gonad (ovaries) | RNAlater |
| heart, basal | RNAlater |
| heart, apex | RNAlater |
| Kidney | RNAlater, Formalin Fixed |
| Liver | RNAlater, Formalin Fixed, |
| | Flash Frozen |
| lymph nodes, cervical | RNAlater |
| Neural Retinal | Flash Frozen, Formalin Fixed |
| skeletal muscle, gastrocnemius | RNAlater |
| skeletal muscle, quadriceps | RNAlater |
| spinal cord (cervical) | Flash Frozen, Formalin Fixed |
| spinal cord (thoracic) | Flash Frozen, Formalin Fixed |
| spinal cord (lumbar) | Flash Frozen, Formalin Fixed |
| spinal cord (sacral) | Flash Frozen, Formalin Fixed |
| Spleen | RNAlater |
| carotid artery | RNA/ater |

### 7.2.1.3. Tissue Biodistribution And Transduction Analysis

Left Hemisphere Flash Frozen brain slices were dissected to isolate regions including, but not limited to, frontal cortex, temporal cortex, motor cortex, hippocampus, basal ganglia, midbrain, brainstem, and cerebellum. Liver, Spleen, and DRG samples were also analyzed. For biodistribution and transduction analyses, total DNA and RNA was extracted from tissue samples with Trizol/chloroform and isopropanol precipitation. Reverse transcription was done with Protoscript II Reverse Transcriptase (NEB) with primers that were specific to the vector transgene and transcripts included the unique molecular identifiers (UMIs). Control reactions lacking the reverse transcriptase enzyme (-RT control) were also prepared. Quantification of biodistribution and transduction was done with Luna Universal Probe qPCR Master Mix (NEB) using primers and probes specific to the transgene construct. Finally, samples were prepared for next-generation sequencing by amplifying the transgene barcode regions with primers compatible with Illumina NGS platform and sequenced with NextSeq 550 (Illumina).

After sequencing, the barcode tags were extracted from reads with the expected amplicon structure, and the abundance (number of reads or number of UMIs) of each barcode was recorded. Analyses were restricted to the set of barcodes that were present in the input plasmid sample, as measured by a separate sequencing assay that targeted the variant regions of the input plasmid sample.

To aggregate packaging replicates, the read counts from replicate virus production samples were summed. To aggregate biodistribution samples, read counts from samples from the same tissue were summed. To aggregate transduction samples, the UMI counts from samples from the same tissue were summed.

### 7.2.1.4. NHP Tissue Immunofluorescence

Formalin fixed right hemisphere brain slices, as well as DRG and spinal cord, were paraffin embedded, sliced at 5 uM, and stained for NeuN (neuronal marker), mCherry, and GFP. The NeuN/GFP/mCherry staining was performed on the Leica BOND RX Autostainer. The primary antibodies used include GFP (PA5-34974, Thermo-Fisher), mCherry (Ab6556, Abcam), and NeuN (Ab177487, Abcam). Immunofluorescence-stained tissue 1″×3″ glass slides were scanned using an Akoya Vectra Polaris Fluorescent scanner to produce whole-slide digital images. The number of total cells staining for GFP (VAR-B1) and mCherry (AAV9), as well as the number of VAR-B1 and AAV9 positive cells co-staining with NeuN (to ascertain transduction and expression in neurons), were manually counted in the high dose animal using QuPath software. Regions of interest (ROI) analyzed included the hippocampus, frontal cortex, caudate, cerebellum, and spinal cord. All areas were analyzed in duplicate (2 slides each), except spinal cord which was analyzed in triplicate. All ROIs were counted in their entirety on the slide except the cerebellum where 2 cortical folds were analyzed per slice.

### 7.2.2. Results

### 7.2.2.1. Bulk Tissue Analysis by NGS sequencing of viral RNA (transduction) and viral DNA (biodistribution)

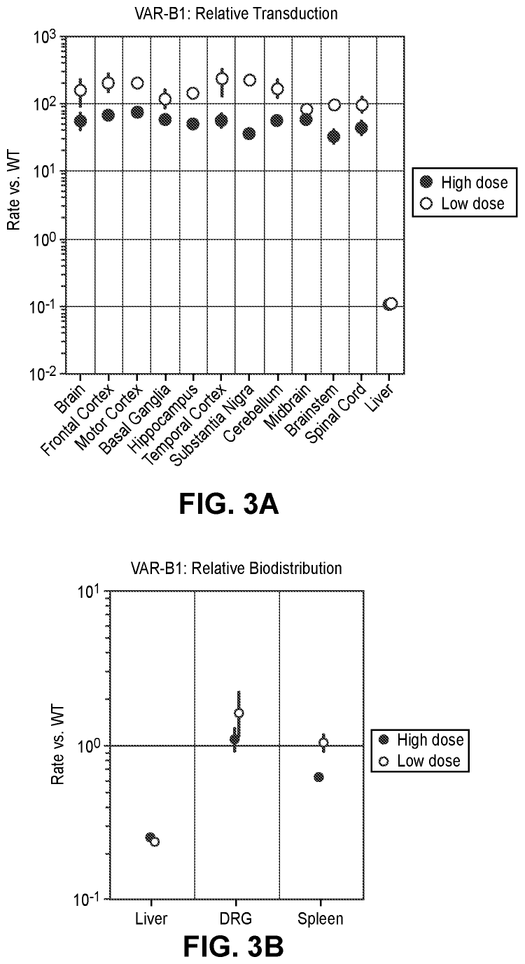

VAR-B1 shows increased transduction across multiple brain regions compared to WT AAV9 in this two-capsid, direct comparison study. The number of samples analyzed by NGS in each animal and brain region is listed in Tables 8 and 9 and the property measurements for VAR-B1, averaged across all tissue pieces for the indicated tissue type for each NHP, are reported in Table 10 and in FIG. 3A (transduction relative to wild-type AAV9) and FIG. 3B (biodistribution relative to wild-type AAV9). In FIG. 3A and Table 10, “brain transduction” represents an aggregated measurement of relative transduction averaged across all collected brain samples. The high dose resulted in a 56.73-fold increase in brain transduction overall with a range of 34- to 76-fold increase in different regions (Table 10 and FIG. 3A). The low dose resulted in a relative increase to WT AAV9 of 161-fold for total brain transduction, ranging from 82- to 213-fold increase in different brain regions (Table 10 and FIG. 3A). Assessment of off-target tissues showed that VAR-B1 exhibits reduced liver biodistribution at 0.26- and 0.23-fold AAV9 in high and low doses respectively, and liver transduction was 0.11-fold WT AAV9 for both doses (Table 10 and FIGS. 3A and 3B). Biodistribution of VAR-B1 in the DRG was largely unchanged from WT AAV9 with a 1.1- and 1.6-fold increase to AAV9 observed in high and low doses, respectively (Table 10 and FIG. 3B). Spleen biodistribution was unchanged at the low dose but decreased to 0.63-fold AAV9 at the high dose (FIG. 3B). These results confirm the improved properties of VAR-B1 exhibited in the high-throughput library experiment reported in Example 1. These results suggest that, without being bound by theory, gene therapies that include the capsid variant described herein is useful for the treatment of CNS disorders, for example as described herein, for example, neurodegenerative disorders such as, but not limited to, Parkinson's Disease, Huntington's disease, Alzheimer's disease, Fragile X, Rett Syndrome, Angelman Syndrome, ataxias and frontotemporal dementia.

TABLE 8
RNA transduction tissue sample number

| Tissue | High Dose | Low Dose |
|---|---|---|
| Frontal Cortex | 1 | 5 |
| Motor Cortex | 5 | 2 |
| Basal Ganglia | 8 | 9 |
| Hippocampus | 3 | 4 |
| Temporal Cortex | 7 | 7 |
| Substantia Nigra | 1 | 1 |
| Cerebellum | 5 | 6 |
| Midbrain | 3 | 2 |
| Brainstem | 6 | 5 |
| Spinal Cord | 5 | 5 |
| Liver | 2 | 2 |

TABLE 9
vDNA biodistribution sample number

| Tissue | High Dose | Low Dose |
|---|---|---|
| Liver | 4 | 4 |
| DRG | 4 | 4 |
| Spleen | 4 | 4 |

TABLE 10
Biodistribution and transduction of VAR-B1. All values reported
fold change relative to WT AAV9. All values are an aggregate
across all tissue samples collected from each NHP animal in
the study. “Brain transduction” is an aggregate across
all brain samples analyzed from each NHP animal in the study.

Fold Change from WT AAV9

| Property | High Dose | Low Dose |
|---|---|---|
| Brain transduction | 56.7314 | 160.8062 |
| Frontal Cortex transduction | 70.7492 | 213.4124 |
| Motor Cortex Transduction | 76.7434 | 196.2372 |
| Basal Ganglia transduction | 61.8081 | 120.7397 |
| Hippocampus transduction | 54.2627 | 139.2863 |
| Temporal Cortex transduction | 57.2538 | 228.6310 |
| Substantia Nigra transduction | 36.3738 | 220.6999 |
| Cerebellum transduction | 57.3525 | 170.9359 |
| Midbrain transduction | 58.4986 | 82.2261 |
| Brainstem transduction | 33.5664 | 95.7057 |
| Spinal cord transduction | 43.7355 | 97.4933 |
| Liver transduction | 0.111524 | 0.112531 |
| Liver biodistribution | 0.255386 | 0.234775 |
| DRG biodistribution | 1.102479 | 1.621922 |
| Spleen biodistribution | 0.625651 | 1.046902 |

### 7.2.2.2. Histology: Immunofluorescence

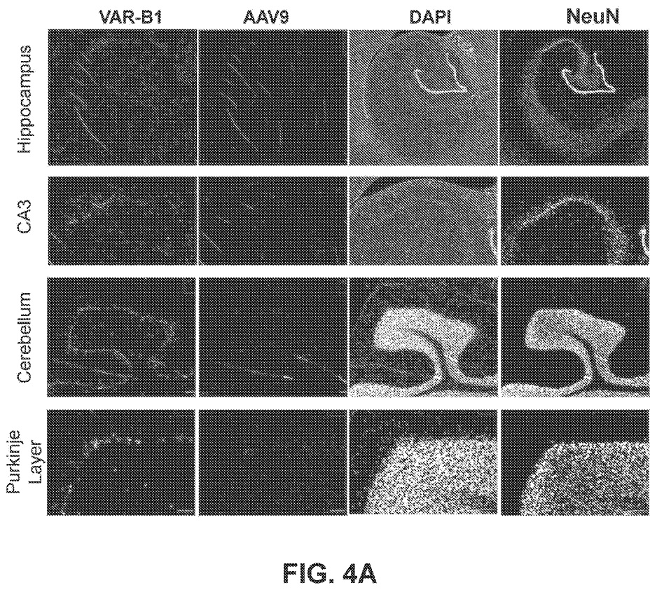

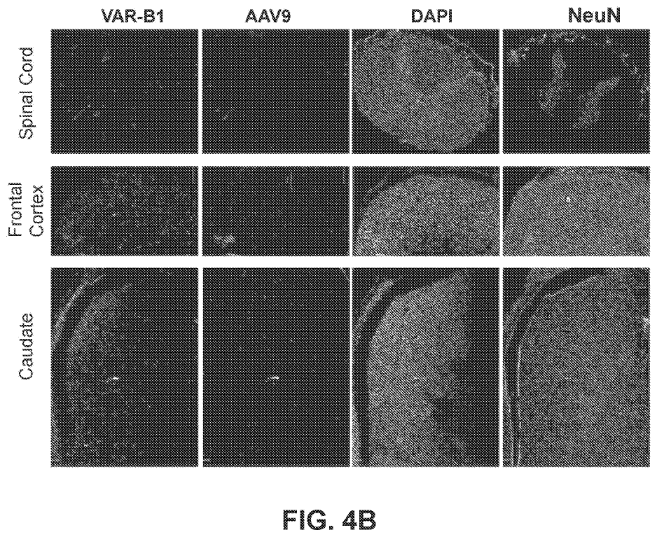

Histological analysis of cell numbers expressing GFP from the VAR-B1 virus particles and mCherry from the wild-type AAV9 virus particles demonstrates that VAR-B1 virus particles have increased transgene expression relative to wild-type AAV9 virus particles throughout the brain areas analyzed from the high dose animal. The fold increase in number of cells stained for VAR-B1 compared to WT AAV9 virus particles transgene expression varies by region, with highest increases in the hippocampus and cerebellar cortex (Table 11, FIGS. 4A and 4B). A consistent increase in the number of neurons stained was also observed for VAR-B1 compared to AAV9, although also varying by area. Large increases in neuronal staining are observed in the frontal cortex and caudate (Table11, FIG. 4B). The hippocampal CA3 also shows distinct staining by transduction from VAR-B1 in the pyramidal neuron layer, with 16.6% of neurons in this region expressing GFP whereas no neurons express mCherry (Table 11, FIG. 4A). Additionally, 5% of the total counted CA3 neurons expressed the VAR-B1 virus particle transgene (GFP). The Purkinje cell layer of the cerebellar cortex also had very high levels of VAR-B1 GFP staining compared to AAV9 mCherry. However, despite having visual appearance of large neurons, Purkinje neurons do not stain with the NeuN antibody used, so neuronal staining cannot be determined (FIG. 4A). The high transduction and cell staining patterns exhibited in the cerebellum suggest that, without being bound by theory, gene therapies that include the capsid variant described herein are particularly useful for the treatment of Spinocerebellar ataxias. The full brain histology staining results suggest that, without being bound by theory, gene therapies that include the capsid variant described herein are useful for the treatment of CNS disorders, e.g., as described herein, e.g., neurodegenerative disorders such as Parkinson's Disease, Huntington's disease, and Alzheimer's disease.

TABLE 11
Cell counts for VAR-B1 virus particle transgene expression (GFP) normalized to
wild-type AAV9 virus particle transgene expression (mCherry) for total cells
and neurons. All values are the reported fold change relative to AAV9 and are
the average of counts in 2 slices for all regions except the cerebellar cortex
(average of 4 cortical folds (2/slice) and spinal cord (3 slices). Not determinable:
fold change not able to be determined due to 0 neurons detected with AAV9 mCherry.
N/A*: Purkinje neurons in the Purkinje layer do not stain with NeuN.

| | Total Cells Fold change | |
|---|---|---|
| | from AAV9 in VAR-B1 | Neuron Fold change from AAV9 in |
| Brain Region | positive cells | VAR-B1 positive cells |
| Hippocampus Total | 25.0 | 4.3 |
| Hippocampus CA3 | 24.7 | Not determinable: no AAV9 staining |
| Frontal Cortex | 15.9 | 25.2 |
| Cerebellum Purkinje Layer | 36.4 | N/A* |
| Cerebellum Granule Layer | 25.3 | 11 |
| Caudate | 4.6 | 37.2 |
| Spinal Cord | 9.4 | Not determinable: no AAV9 staining |

### 7.3. Example 3: In Vitro Evaluation of VAR-B1 and AAV9

In vitro transduction was used to evaluate the ability of WT AAV9 and VAR-B1 virus particles to infect and transduce human neurons. Sh-sy5y and primary neuron cultures were used for this analysis and analyzed for the expression of GFP from the VAR-B1 virus particles and mCherry from the WT AAV9 virus particles produced in Example 2.

### 7.3.1. Materials and Methods

### 7.3.1.1. Sh-sy5y In Vitro Transduction Assay

The assays were performed in 12 well plates with poly-l-lysine coated coverslips. Sh-sy5y (human glioblastoma cell line, Sigma, 94030304-1VL) cells were plated at a density of 300,000 cells per well. 24 hours post plating the virus particles were added to the cells at MOIs of 100,000, 50,000, and 10,000 vg/cell in media lacking FBS and incubated on ice for 1 hour. After one hour, complete media was added to the cells. Four days post transduction the cells were fixed with 4% paraformaldehyde and stained by immunofluorescence for MAP2 (1:1000 Abcam ab5392) and NeuN (1:500, Sigma Aldrich ABN78), utilizing Alexa 595 and 405 conjugated (1:1000) secondary antibodies. GFP was visualized using the native fluorescence from the capsid transgene.

### 7.3.1.2. Primary Human Neurons In Vitro Transduction Assay

The assays were performed in 12 well plates with poly-l-lysine coated coverslips. Primary Human Neurons (Sciencell #1530) were plated at 8.3e4 cells per well. The cells were grown for 7 days in a 37° C. incubator with media changes every two days. At 7 days in vitro the virus particles were added to the cells at MOIs of 10,000, 25,000, or 50,000 vg/cell and incubated at 370C for 1 hour. After one hour incubation complete media was added, including all the required supplements. Seven days post-transduction the cells were fixed with 4% paraformaldehyde and stained/visualized using MAP2 (1:1000 Abcam ab5392), native GFP or mCherry fluorescence, and DAPI (Vectashield NC1601055).

### 7.3.1.3. Image Acquisition

Images were acquired on an EVOS M5000 with a 20x objective. The images were quantified using ImageJ plugin cell count. The counts were averaged across 5-7 fields of view from two different coverslips.

### 7.3.2. Results

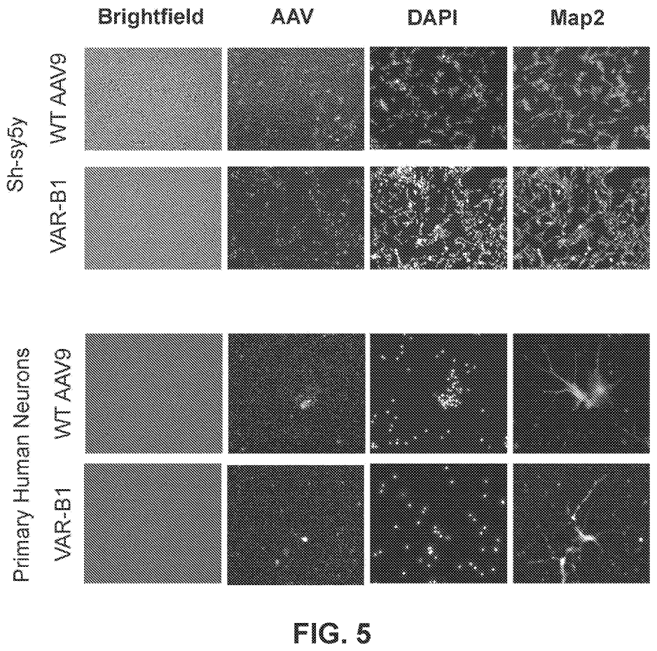

The in vitro transduction results are summarized in Table 12. VAR-B1 virus particles transduce Primary Human Neurons at a rate of 8.65% (50K MOI) and Sh-sy5y glioblastoma neurons at 37.65% (100K MOI). VAR-B1 also transduces human neurons at a greater abundance than WT AAV9 in both cell types with a 3.7-fold increase compared to WT in Sh-sy5y (100K MCI) and 1.6-fold increase in primary human neurons (50K MOI). Representative images (brightfield, transgene expression, and cell markers) from sh-sy5y and primary human neurons treated with VAR-B1 and WT AAV9 virus particles are shown in FIG. 5. These results demonstrate that VAR-B1 has the ability to transduce human neurons in vitro, supporting a translational and clinical application of this capsid in gene therapies targeting CNS disorders, for example as described herein.

TABLE 12
Cell transduction of VAR-B1 and WT AAV9 quantification
in Sh-sy5y and Primary Human Neurons

| | | MOI | Cells | % Cells | Standard |
|---|---|---|---|---|---|
| Cell Type | Test Article | Transduced | Counted | Transduced | Error |
| Primary Human | WT AAV9 | 10K MOI | 403 | 1.74 | 1.1 |
| Neurons | | 25K MOI | 284 | 3.26 | 0.95 |
| | | 50K MOI | 1033 | 5.5 | 2.26 |
| | VAR-B1 | 10K MOI | 551 | 1.42 | 0.61 |
| | | 25K MOI | 590 | 3.8 | 1.7 |
| | | 50K MOI | 577 | 8.65 | 2.9 |
| Sh-sy5y | WT AAV9 | 10K MOI | 1201 | 1.74 | 2.5 |
| | | 50K MOI | 1955 | 11.19 | 5.1 |
| | | 100K MOI | 940 | 10.14 | 3.7 |
| | VAR-B1 | 10K MOI | 2552 | 6.63 | 0.65 |
| | | 50K MOI | 1581 | 28.64 | 2.216 |
| | | 100K MOI | 2868 | 37.69 | 1.66 |

### 7.4. Example 4: High-Throughput Evaluation of VAR-B1 Additional Capsid Variants

### 7.4.1. Materials and Methods

### 7.4.1.1. Library #2 Creation

A library of 1E5 capsid variants of wild-type AAV9 (“Library #2”) was designed with the goals of producing a capsid that would package into AAV particles, transduce central nervous system tissues after intravenous injection with high efficiency, and de-target the liver and other tissue types. This library was designed and synthesized according to Example 1, with the following variations. First, a subset of these variants included in this library is 126 capsid polypeptide variants that are identical to the sequence of wild-type AAV9, but contain various subcombinations of the amino acid mutations in the mutation set of VAR-B1. The capsid polypeptide VP1 sequences are provided in Table 13 below and as SEQ ID NOs:137-252 in Table 37 (“subcombination variant set”).

TABLE 13

| VAR-# | Mutation set as compared to SEQ ID NO:1 |
|---|---|
| VAR-B1 | [‘Q579V’, ‘Q592I’, ‘T593V’, ‘W595A’, ‘V596L’, ‘N598S’, ‘I601A’] |
| VAR-B54 | [“Q579V”, “T593V”, “W595A”, “V596L”, “N598S”, “I601A”] |
| VAR-B74 | [“Q579V”, “T593V”, “V596L”, “N598S”, “I601A”] |
| VAR-B87 | [“Q579V”, “W595A”, “V596L”, “N598S”, “I601A”] |
| VAR-B61 | [“Q592I”, “T593V”, “V596L”, “N598S”, “I601A”] |
| VAR-B116 | [“Q579V”, “Q592I”, “W595A”, “V596L”, “N598S”, “I601A”] |
| VAR-B100 | [“Q592I”, “T593V”, “W595A”, “V596L”, “N598S”, “I601A”] |
| VAR-B80 | [“T593V”, “V596L”, “N598S”, “I601A”] |
| VAR-B89 | [“T593V”, “W595A”, “V596L”, “N598S”, “I601A”] |
| VAR-B102 | [“Q579V”, “Q592I”, “T593V”, “V596L”, “N598S”, “I601A”] |
| VAR-B122 | [“Q579V”, “Q592I”, “T593V”, “W595A”, “V596L”, “N598S”] |
| VAR-B75 | [“Q579V”, “T593V”, “W595A”, “V596L”, “N598S”] |
| VAR-B96 | [“Q579V”, “T593V”, “V596L”, “N598S”] |
| VAR-B67 | [“Q579V”, “V596L”, “N598S”, “I601A”] |
| VAR-B57 | [“Q579V”, “Q592I”, “V596L”, “N598S”, “I601A”] |
| VAR-B59 | [“Q592I”, “T593V”, “V596L”, “N598S”] |
| VAR-B35 | [“Q579V”, “Q592I”, “T593V”, “W595A”, “N598S”, “I601A”] |
| VAR-B110 | [“Q592I”, “T593V”, “V596L”] |
| VAR-B93 | [“Q592I”, “T593V”, “W595A”, “V596L”, “N598S”] |
| VAR-B76 | [“Q579V”, “W595A”, “I601A”] |
| VAR-B119 | [“Q592I”, “V596L”, “N598S”, “I601A”] |
| VAR-B82 | [“Q579V”, “W595A”, “V596L”, “N598S”] |
| VAR-B83 | [“V596L”, “N598S”, “I601A”] |
| VAR-B108 | [“Q579V”, “Q592I”, “T593V”, “W595A”, “I601A”] |
| VAR-B71 | [“W595A”, “V596L”, “N598S”, “I601A”] |
| VAR-B90 | [“Q579V”, “Q592I”, “W595A”, “V596L”, “N598S”] |
| VAR-B123 | [“Q579V”, “T593V”, “W595A”, “V596L”] |
| VAR-B44 | [“Q592I”, “N598S”] |
| VAR-B86 | [“Q579V”, “V596L”] |
| VAR-B81 | [“Q579V”, “V596L”, “N598S”] |
| VAR-B43 | [“T593V”, “N598S”, “I601A”] |
| VAR-B41 | [“Q579V”, “Q592I”, “W595A”, “N598S”] |
| VAR-B88 | [“Q579V”, “T593V”, “V596L”] |
| VAR-B127 | [“Q592I”, “W595A”, “I601A”] |
| VAR-B85 | [“V596L”, “N598S”] |
| VAR-B51 | [“Q592I”, “T593V”, “W595A”] |
| VAR-B99 | [“Q579V”, “Q592I”, “V596L”, “N598S”] |
| VAR-B101 | [“Q592I”, “V596L”] |
| VAR-B107 | [“Q579V”, “Q592I”, “T593V”, “W595A”, “V596L”] |
| VAR-B5 | [“Q579V”, “T593V”] |
| VAR-B33 | [“Q579V”, “T593V”, “W595A”, “N598S”] |
| VAR-B3 | [“I601A”] |
| VAR-B25 | [“Q579V”, “Q592I”, “T593V”, “W595A”, “N598S”] |
| VAR-B49 | [“Q579V”, “Q592I”, “T593V”, “I601A”] |
| VAR-B68 | [“Q579V”, “W595A”, “V596L”] |
| VAR-B40 | [“T593V”, “W595A”, “N598S”, “I601A”] |
| VAR-B97 | [“Q579V”, “W595A”, “V596L”, “I601A”] |
| VAR-B10 | [“N598S”] |
| VAR-B105 | [“Q579V”, “T593V”, “W595A”, “N598S”, “I601A”] |
| VAR-B114 | [“T593V”, “W595A”, “V596L”, “N598S”] |
| VAR-B69 | [“Q579V”, “Q592I”, “W595A”, “V596L”, “I601A”] |
| VAR-B14 | [“V596L”] |
| VAR-B70 | [“V596L”, “I601A”] |
| VAR-B55 | [“T593V”, “W595A”, “V596L”] |
| VAR-B78 | [“T593V”, “W595A”, “V596L”, “I601A”] |
| VAR-B50 | [“Q579V”, “Q592I”, “N598S”] |
| VAR-B53 | [“Q592I”, “T593V”, “I601A”] |
| VAR-B77 | [“Q579V”, “T593V”, “W595A”, “V596L”, “I601A”] |
| VAR-B109 | [“Q579V”, “W595A”, “N598S”, “I601A”] |
| VAR-B112 | [“W595A”, “V596L”, “N598S”] |
| VAR-B124 | [“Q579V”, “N598S”] |
| VAR-B46 | [“Q579V”, “Q592I”, “W595A”, “N598S”, “I601A”] |
| VAR-B28 | [“Q579V”, “T593V”, “W595A”] |
| VAR-B17 | [“T593V”, “W595A”] |
| VAR-B47 | [“Q592I”, “T593V”, “N598S”] |
| VAR-B62 | [“Q579V”, “Q592I”, “I601A”] |
| VAR-B2 | [“N598S”, “I601A”] |
| VAR-B30 | [“Q592I”, “T593V”, “N598S”, “I601A”] |
| VAR-B103 | [“W595A”, “N598S”, “I601A”] |
| VAR-B72 | [“Q579V”, “Q592I”, “T593V”, “V596L”] |
| VAR-B23 | [“Q579V”, “Q592I”, “T593V”] |
| VAR-B95 | [“T593V”, “V596L”, “N598S”] |
| VAR-B73 | [“T593V”, “V596L”, “I601A”] |
| VAR-B79 | [“Q592I”, “V596L”, “N598S”] |
| VAR-B115 | [“Q579V”, “Q592I”, “V596L”, “I601A”] |
| VAR-B98 | [“Q579V”, “V596L”, “I601A”] |
| VAR-B125 | [“Q592I”, “W595A”, “V596L”, “I601A”] |
| VAR-B104 | [“Q592I”, “T593V”, “W595A”, “V596L”] |
| VAR-B31 | [“Q579V”, “T593V”, “N598S”] |
| VAR-B24 | [“Q592I”, “W595A”] |
| VAR-B39 | [“Q579V”, “Q592I”, “T593V”, “N598S”, “I601A”] |
| VAR-B8 | [“Q579V”, “I601A”] |
| VAR-B11 | [“Q592I”, “T593V”] |
| VAR-B32 | [“Q579V”, “Q592I”, “W595A”] |
| VAR-B6 | [“W595A”] |
| VAR-B121 | [“Q592I”, “W595A”, “V596L”, “N598S”] |
| VAR-B9 | [“W595A”, “V596L”] |
| VAR-B65 | [“Q592I”, “T593V”, “W595A”, “V596L”, “I601A”] |
| VAR-B21 | [“Q579V”, “Q592I”] |
| VAR-B58 | [“Q579V”, “Q592I”, “W595A”, “V596L”] |
| VAR-B34 | [“Q579V”, “Q592I”, “T593V”, “W595A”] |
| VAR-B120 | [“Q592I”, “W595A”, “V596L”] |
| VAR-B118 | [“W595A”, “V596L”, “I601A”] |
| VAR-B27 | [“Q592I”, “W595A”, “N598S”] |
| VAR-B29 | [“T593V”, “N598S”] |
| VAR-B4 | [“T593V”] |
| VAR-B7 | [“Q579V”, “W595A”] |
| VAR-B12 | [“Q592I”] |
| VAR-B13 | [“Q579V”] |
| VAR-B15 | [“W595A”, “N598S”] |
| VAR-B16 | [“W595A”, “I601A”] |
| VAR-B18 | [“T593V”, “W595A”, “I601A”] |
| VAR-B19 | [“T593V”, “I601A”] |
| VAR-B20 | [“Q592I”, “W595A”, “N598S”, “I601A”] |
| VAR-B22 | [“Q592I”, “I601A”] |
| VAR-B26 | [“Q579V”, “W595A”, “N598S”] |
| VAR-B36 | [“Q579V”, “Q592I”, “T593V”, “N598S”] |
| VAR-B37 | [“Q579V”, “Q592I”, “W595A”, “I601A”] |
| VAR-B38 | [“Q579V”, “Q592I”, “N598S”, “I601A”] |
| VAR-B42 | [“Q592I”, “T593V”, “W595A”, “N598S”] |
| VAR-B45 | [“Q579V”, “T593V”, “N598S”, “I601A”] |
| VAR-B48 | [“Q579V”, “N598S”, “I601A”] |
| VAR-B52 | [“Q592I”, “N598S”, “I601A”] |
| VAR-B56 | [“T593V”, “W595A”, “N598S”] |
| VAR-B60 | [“T593V”, “V596L”] |
| VAR-B63 | [“Q592I”, “W595A”, “V596L”, “N598S”, “I601A”] |
| VAR-B64 | [“Q592I”, “T593V”, “V596L”, “I601A”] |
| VAR-B66 | [“Q579V”, “Q592I”, “T593V”, “W595A”, “V596L”, “I601A”] |
| VAR-B84 | [“Q579V”, “T593V”, “V596L”, “I601A”] |
| VAR-B91 | [“Q579V”, “Q592I”, “T593V”, “V596L”, “N598S”] |
| VAR-B92 | [“Q579V”, “T593V”, “W595A”, “I601A”] |
| VAR-B94 | [“Q579V”, “Q592I”, “V596L”] |
| VAR-B106 | [“Q579V”, “T593V”, “I601A”] |
| VAR-B111 | [“Q579V”, “Q592I”, “T593V”, “V596L”, “I601A”] |
| VAR-B113 | [“Q592I”, “T593V”, “W595A”, “N598S”, “I601A”] |
| VAR-B117 | [“Q592I”, “V596L”, “I601A”] |
| VAR-B126 | [“Q592I”, “T593V”, “W595A”, “I601A”] |

Without being bound by theory, these variants were designed to specifically characterize the minimally active set of mutations responsible for the enhanced activity of VAR-B1. In addition, the library contained a selection of variants which, relative to wild-type AAV9, included between 1 and 3 amino acid mutations in addition to the mutations of the mutation set of VAR-B1. Without being bound by theory, these additional variants were designed to provide evidence confirming the enhanced performance of virus particles comprising the mutation sets described herein, for example, of VAR-B1, in different sequence contexts. Second, the library was cloned twice using two different versions of the viral genome, one with expression of the capsid gene under the control of a ubiquitous CBh promoter and the other with expression of the capsid gene under the control of a neuronal specific hSyn promoter. Each plasmid backbone contained a unique genomic identifier enabling analysis of biodistribution and transduction efficiencies of each capsid variant via each of the two promoters.

### 7.4.1.2. In Vitro Evaluation of Library #2

In vitro evaluation of the library #2 was completed as outlined in Example 1.

### 7.4.1.3. In Vivo Evaluation of Library in Non-Human Primate

All NHP experiments were conducted in accordance with institutional policies and NIH guidelines. Two female Cynomolgus Macaque primates weighing 2.5 and 2.6 kg and seronegative for anti-AAV9 neutralizing antibodies (serum NAb titers <1:4 based on in vitro NAb assay) were selected for the study. Animals were treated with Methylprednisolone (40 or 80 mg IM) on Day −8 and weekly for the duration of the study. Prior to test article administration, samples of blood were collected. The animals received an intravenous injection of a mixture of the promoter vector libraries (total combined dose for each animal: 8.64 e13 vg/kg). During the in-life period the animals were monitored according to the animal facility's SOPs. Serum samples were collected at 2 days, 4 days, 7 days, and weekly after the injections. The animals were sacrificed 4 weeks after the injections and tissues were collected and analyzed as described in Example 1 for biodistribution and transduction. The tissues collected are shown in Table 14.

TABLE 14
List of tissue collected
Tissue

Adrenal gland
Adipose Tissue
Aorta
Brain coronal axis slices
Carotid artery
Dorsal Root Ganglion (Cervical)
Dorsal Root Ganglion (Thoracic)
Dorsal Root Ganglion (Lumbar)
Dorsal Root Ganglion (Sacral)
Gonad (Ovaries)
Heart, Basal
Heart, Apex
Heart, Ventricle
Kidney
Liver
Lung
Lymph Nodes, Cervical
Optic Nerve
Sciatic Nerve
Skeletal Muscle, Gastrocnemius
Skeletal Muscle, Quadriceps
Skeletal Muscle, Diaphragm
Spinal Cord (Cervical)
Spinal Cord (Cranial Thoracic)
Spinal Cord (Caudal Thoracic)
Spinal Cord (Lumbar)
Spleen

After sequencing, the barcode tags were extracted from reads with the expected amplicon structure, and the abundance (number of reads or number of UMIs) of each barcode was recorded. Analyses were restricted to the set of barcodes that were present in the input plasmid sample, as measured by a separate sequencing assay that targeted the variant regions of the input plasmid sample.

To aggregate packaging replicates, the read counts from replicate virus production samples were summed. To aggregate biodistribution samples, read counts from samples from the same tissue were summed. To aggregate transduction samples, the number of transduction events (measured by unique id tags detected) from samples from the same tissue were summed.

Virus packaging was calculated by normalizing aggregated production replicates with input plasmid abundance. Biodistribution and transduction of tissue were calculated by normalizing aggregated biodistribution or transduction samples with input virus abundance. The output was reported as a fold change relative to the WT AAV9.

### 7.4.2. Results

The production, biodistribution and transduction results for VAR-B1 and the subcombination variant set from this library experiment are summarized in Table 16 (production), Table 17 (CNS biodistribution), Table 18 (periphery biodistribution), Table 19 (CNS transduction) and Table 20 (periphery transduction). As was the case in the library experiment described in Example 1 and the single capsid experiment described in Example 2, VAR-B1 displays increased transduction and biodistribution across the brain regions sampled compared to AAV9. This was observed for both promoters with brain transduction having a 138-fold and 129-fold increase compared to WT AAV9 with the CBh and hSyn promoters, respectively. Additionally, periphery VAR-B1 biodistribution and transduction in the liver is decreased to 0.3-fold AAV9 and biodistribution was also decreased in the spleen, again confirming the results of the previous experiments. HEK293 virus production was also suitable in this study. Taken together, these findings further indicate that VAR-B1 is suitable for gene therapies where targeting the brain is important, for example, as described herein.

In addition to VAR-B1, other VAR-B1-like capsid polypeptides (e.g., containing subcombinations of the mutation set of VAR-B1) exhibited similar transduction profiles to VAR-B1 with elevated transduction across the brain. As shown in Table 19, of the 93 variants where brain transduction data could be captured, 77 of the variants with subcombinations of the VAR-B1 mutation set exhibited better than WT AAV9 transduction in aggregated brain regions assessed when transgene expression was under the control of the neuronal specific hSyn promoter, and 79 of the 93 variants measured showed better than WT AAV9 transduction in aggregated brain regions when expression was under the control of the ubiquitous CBh promoter

Assessment of the set of variants with subcombinations of the VAR-B1 mutation set revealed several sequences which represent minimal sets of mutations conferring enhanced brain transduction of variant capsid polypeptides. In particular, VAR-B80 (having 4 mutations in its mutation set: [“T593V”, “V596L”, “N598S”, and “1601A” ]) exhibited 22.4- and 32.2-fold increases in aggregated brain transduction relative to wild-type AAV9 from the CBh and hSyn promoters, respectively. In addition, the mutations V596L, N598S and 1601A are entirely conserved in the top 10 subcombination set variants when ranked by aggregated brain expression from the hSyn promoter, and the variant comprising only these three mutations (VAR-B83) exhibits greater than 5-fold increased transduction in aggregated brain relative to wild-type AAV9. Another highly conserved motif in the top aggregated brain transducers is the combination of W595A, V596L, and N598S. The variant containing only these three mutations in its mutation set (VAR-B112) exhibited nearly 2-fold increased transduction relative to wild-type AAV9 in aggregated brain tissue. Interestingly, the variant which also includes the Q579V mutation to this mutation set (VAR-B82) exhibited a 6.5-fold increase in aggregated brain transduction relative to AAV9 from the hSyn promoter, indicating that the combination of valine at 579, alanine at position 595, leucine at position 596 and serine at position 589 confers enhanced brain transduction properties on virus particles. Another motif which emerged was the combination of Q5921, T593V and V596L. The variant having only these mutations in its mutation set (VAR-B110) was the strongest transducing variant with only 3 mutations relative to wtAAV9, exhibiting 8.2-fold increase in aggregated brain transduction from the hSyn promoter relative to wild-type AAV9. Another motif which emerged was the combination of T593V, W595A, V596L, and N598S. 5 of the 10 ten variants ranked according to fold improvement over wild-type AAV9 in aggregated brain transduction from the hSyn promoter included this mutation set and the variant with only these 4 mutations (VAR-B114) exhibited 2.18-fold aggregate brain transduction over wild-type AAV9 from the hSyn promoter and 1.06-fold aggregate brain transduction from the CBh promoter. Again, the results show an improved effect when combining the mutation sets: for example, when these 4 mutations (T593V, W595A, V596L, and N598S) are included with the Q579V mutation (VAR-B75), virus particles show 17.07-fold and 18.85-fold aggregated brain transduction relative to wild-type AAV9 from the CBh and hSyn promoters, respectively. Since these minimal motifs independently lead to increased brain transduction, without being bound by theory, these motifs are additive and/or synergistic, and can lead to enhanced effects when combined.

Of the variants exhibiting greater than 5-fold aggregated brain transduction from the hSyn promoter, 6 variants had 6 of the 7 mutations of the VAR-B1 mutation set; 9 variants had 5 of the 7 mutations of the VAR-B1 mutation set; 7 variants had 4 of the 7 mutations of the VAR-B1 mutation set; and 3 variants had 3 of the 7 mutations of the VAR-B1 mutation set. This indicates that variants which contain 3, 4, 5, 6 or 7 of the mutations in the mutation set of VAR-B1 are capable of transducing cells of the CNS upon intravenous injection, indicating the potential to cross the blood brain barrier and reach cells of interest to treat CNS disorders for example as described herein.

Finally, an additional set of variants were designed which included the mutation set of VAR-B1 and which included between 1 and 3 additional mutations. Of the variants from this set, all but one of the designed variants (18 of 19 designed variants) which were detected in at least one brain region exhibited transduction levels of at least 1.75-fold over wild-type AAV9 in aggregated brain regions from the hSyn promoter. Fold increases in these 19 variants in aggregated brain samples ranged from a 1.75-fold improvement to an over 134-fold improvement. Without being bound by theory, these results indicate that the mutation set of VAR-B1 is active in providing increased brain transduction when present in diverse sequence contexts.

When assessing the data by individual brain region, 3 variants other than VAR-B1; VAR-B54, VAR-B74, and VAR-B87, exhibit high transduction in the temporal cortex. Additionally, VAR-B61 exhibits consistent increased transduction for the hSyn neuronal promoter compared to the CBh promoter (relative to AAV9). This result suggests that this variant could have more neuronal-specific characteristics and without being bound by theory, gene therapies that include the capsid variants described herein are useful for the treatment of neuronal specific disorder such as Rett Syndrome, Angelman syndrome, Fragile-X, or Tay-Sachs disease, as well as broader neurodegenerative diseases.

Another important finding is based on the fact the experiments described in Example 1 and Example 4 were completed in AGM and Cynomolgus macaque non-human primates, respectively, thereby enabling comparison of VAR-B1 across 2 different genera of NHP. VAR-B1 transduction compared to AAV9 in AGM (Exp 1) and Cyno (Exp 4) are shown in Table 15. Similar levels of brain transduction were observed in both species. In the experiment described in Example 4, in Cynos, VAR-B1 did show slightly increased transduction across the brain (with the exception of spinal cord) compared to the results in AGM. However, differences between species/experiment are minor and suggest that the method by which VAR-B1 increases brain transduction is conserved across NHP species of 2 different genera. These findings further indicate that VAR-B1 is suitable for gene therapies where targeting the brain is important, and in particular in other related species such as humans, for example, as described herein.

TABLE 15
Aggregated brain transduction of VAR-B1 in two separate
high-throughput library experiments, one in AGM (Example
1) and one in Cynomolgus macaque (Example 4). Relative
fold transduction increase to AAV9 is conserved across
species for multiple regions.

| | Fold Transduction for VAR-B1 relative to AAV9 | |
|---|---|---|
| | Results from | |
| Brain region | Example. 1: AGM | Results from Example 4: Cyno |
| Whole brain | 78x (63-96x) | 139x (75-300x) |
| Cerebellum | 96x | 122x |
| Cortex | 88x | 129x |
| Spinal cord | 66x | 46x |

TABLE 16
Production efficiency in HEK293 cells in vitro, relative to production
efficiency of WT AAV9, ranked from highest to lowest. CBh indicates
virus particles comprising genomes with the cap gene under the control
of the CBh promoter. hSyn indicates virus particles comprising genomes
with the cap gene under the control of the hSyn promoter. Variants
are ranked in order of highest fold change to lowest fold change
from the hSyn-containing virus particles.

| | CBh | hSyn |
|---|---|---|
| | Fold Change | Fold Change |
| | Relative to | Relative to |
| VAR-# | wtAAV9 | wtAAV9 |
| VAR-B10 | 1.19 | 1.41 |
| VAR-B14 | 1.26 | 1.17 |
| VAR-B114 | 1.35 | 0.99 |
| VAR-B124 | 1.16 | 0.97 |
| VAR-B9 | 1.21 | 0.96 |
| VAR-B15 | 0.93 | 0.94 |
| VAR-B67 | 0.66 | 0.91 |
| VAR-B82 | 1.32 | 0.91 |
| VAR-B121 | 1.18 | 0.89 |
| VAR-B13 | 1.22 | 0.88 |
| VAR-B93 | 1.11 | 0.87 |
| VAR-B81 | 1.8 | 0.87 |
| VAR-B104 | 0.92 | 0.87 |
| VAR-B7 | 1.16 | 0.87 |
| VAR-B63 | 0.55 | 0.87 |
| VAR-B87 | 0.54 | 0.83 |
| VAR-B68 | 1.2 | 0.83 |
| VAR-B24 | 1.22 | 0.83 |
| VAR-B38 | 0.44 | 0.83 |
| VAR-B83 | 0.7 | 0.82 |
| VAR-B85 | 1.63 | 0.82 |
| VAR-B112 | 1.32 | 0.81 |
| VAR-B120 | 1.2 | 0.81 |
| VAR-B32 | 0.89 | 0.78 |
| VAR-B79 | 1.09 | 0.77 |
| VAR-B101 | 0.91 | 0.76 |
| VAR-B98 | 0.51 | 0.76 |
| VAR-B6 | 0.94 | 0.76 |
| VAR-B21 | 1.07 | 0.76 |
| VAR-B75 | 1.16 | 0.75 |
| VAR-B116 | 0.53 | 0.74 |
| VAR-B28 | 0.94 | 0.74 |
| VAR-B27 | 1.18 | 0.74 |
| VAR-B57 | 0.76 | 0.73 |
| VAR-B96 | 1.44 | 0.72 |
| VAR-B90 | 1.21 | 0.72 |
| VAR-B58 | 1.05 | 0.72 |
| VAR-B42 | 0.93 | 0.72 |
| VAR-B48 | 0.65 | 0.72 |
| VAR-B4 | 0.55 | 0.71 |
| VAR-B33 | 0.77 | 0.7 |
| VAR-B95 | 0.77 | 0.7 |
| VAR-B71 | 0.78 | 0.69 |
| VAR-B50 | 1.03 | 0.69 |
| VAR-B44 | 0.91 | 0.68 |
| VAR-B118 | 0.5 | 0.68 |
| VAR-B25 | 0.74 | 0.67 |
| VAR-B70 | 0.55 | 0.67 |
| VAR-B55 | 1.31 | 0.67 |
| VAR-B29 | 0.72 | 0.67 |
| VAR-B89 | 0.51 | 0.66 |
| VAR-B8 | 0.46 | 0.66 |
| VAR-B12 | 0.87 | 0.66 |
| VAR-B56 | 0.83 | 0.66 |
| VAR-B54 | 0.52 | 0.65 |
| VAR-B94 | 0.67 | 0.65 |
| VAR-B119 | 0.54 | 0.64 |
| VAR-B51 | 0.74 | 0.64 |
| VAR-B34 | 0.81 | 0.64 |
| VAR-B88 | 0.73 | 0.62 |
| VAR-B17 | 0.79 | 0.62 |
| VAR-B47 | 0.6 | 0.62 |
| VAR-B2 | 0.41 | 0.62 |
| VAR-B31 | 0.9 | 0.61 |
| VAR-B74 | 0.53 | 0.6 |
| VAR-B123 | 0.95 | 0.58 |
| VAR-B86 | 1.07 | 0.58 |
| VAR-B11 | 0.52 | 0.58 |
| VAR-B22 | 0.24 | 0.58 |
| VAR-B99 | 1.2 | 0.57 |
| VAR-B3 | 0.32 | 0.57 |
| VAR-B109 | 0.28 | 0.57 |
| VAR-B122 | 0.87 | 0.56 |
| VAR-B107 | 0.72 | 0.55 |
| VAR-B115 | 0.41 | 0.55 |
| VAR-B80 | 0.41 | 0.54 |
| VAR-B69 | 0.41 | 0.54 |
| VAR-B77 | 0.4 | 0.54 |
| VAR-B45 | 0.38 | 0.54 |
| VAR-B97 | 0.82 | 0.53 |
| VAR-B78 | 0.33 | 0.53 |
| VAR-B26 | 1.14 | 0.52 |
| VAR-B36 | 0.95 | 0.52 |
| VAR-B41 | 0.84 | 0.5 |
| VAR-B23 | 0.66 | 0.5 |
| VAR-B60 | 0.41 | 0.5 |
| VAR-B84 | 0.42 | 0.5 |
| VAR-B5 | 0.95 | 0.49 |
| VAR-B19 | 0.31 | 0.48 |
| VAR-B106 | 0.28 | 0.48 |
| VAR-B65 | 0.31 | 0.47 |
| VAR-B1 | 0.43 | 0.46 |
| VAR-B66 | 0.39 | 0.46 |
| VAR-B100 | 0.36 | 0.45 |
| VAR-B52 | 0.33 | 0.45 |
| VAR-B61 | 0.29 | 0.44 |
| VAR-B105 | 0.3 | 0.44 |
| VAR-B73 | 0.11 | 0.44 |
| VAR-B117 | 0.46 | 0.44 |
| VAR-B62 | 0.35 | 0.43 |
| VAR-B59 | 0.39 | 0.42 |
| VAR-B43 | 0.36 | 0.42 |
| VAR-B103 | 0.35 | 0.42 |
| VAR-B39 | 0.37 | 0.42 |
| VAR-B125 | 0.39 | 0.41 |
| VAR-B53 | 0.24 | 0.39 |
| VAR-B30 | 0.28 | 0.39 |
| VAR-B102 | 0.37 | 0.38 |
| VAR-B40 | 0.29 | 0.38 |
| VAR-B110 | 0.26 | 0.37 |
| VAR-B20 | 0.39 | 0.36 |
| VAR-B49 | 0.31 | 0.35 |
| VAR-B46 | 0.32 | 0.34 |
| VAR-B113 | 0.37 | 0.33 |
| VAR-B72 | 0.54 | 0.31 |
| VAR-B111 | 0.34 | 0.31 |
| VAR-B35 | 0.4 | 0.29 |
| VAR-B76 | 0.26 | 0.29 |
| VAR-B18 | 0.22 | 0.28 |
| VAR-B108 | 0.19 | 0.26 |
| VAR-B91 | 0.34 | 0.26 |
| VAR-B16 | 0.19 | 0.25 |
| VAR-B37 | 0.29 | 0.25 |
| VAR-B92 | 0.24 | 0.25 |
| VAR-B126 | 0.27 | 0.25 |
| VAR-B64 | 0.15 | 0.23 |
| VAR-B127 | 0.21 | 0.22 |

TABLE 17
Fold change in biodistribution of virus particles comprising the indicated variant capsid polypeptide, relative to the biodistribution of virus particles comprising WT AAV9 for brain regions.
“Aggregated” is an average across all other measured areas. CBh indicates measurements from virus particles comprising genomes with the cap gene under the control of the CBh promoter.
hSyn indicates virus particles comprising genomes with the cap gene under the control of the hSyn promoter. Values of “0” indicated variant was not detected in the indicated samples.

ORGAN

| | | | | | | | | | | | | | | | |
|---|---|---|---|---|---|---|---|---|---|---|---|---|---|---|---|
| Brain | Brain | Brain | Brain | Brain | Brain | Brain | Brain | Brain | Brain | Brain | Brain | Brain | Brain | Brain | Brain |

TISSUE

| | | | | | | | | | | | | | | | |
|---|---|---|---|---|---|---|---|---|---|---|---|---|---|---|---|
| Aggre- | Aggre- | Basal | Basal | Brain | Brain | Cere- | Cere- | Fore- | Fore- | Hippo- | Hippo- | Mid- | Mid- | Temporal | Temporal |
| gated | gated | Ganglia | Ganglia | Stem | Stem | bellum | bellum | Brain | Brain | Campus | Campus | Brain | Brain | Cortex | Cortex |

PROMOTER

| VAR-# | CBh | hSyn | CBh | hSyn | CBh | hSyn | CBh | hSyn | CBh | hSyn | CBh | hSyn | CBh | hSyn | CBh | hSyn |
|---|---|---|---|---|---|---|---|---|---|---|---|---|---|---|---|---|
| VAR-B1 | 28.15 | 31.96 | 34.04 | 41.13 | 20.84 | 21.8 | 37.28 | 40.29 | 27.68 | 30.34 | 25.39 | 23.62 | 22.52 | 30.62 | 23.08 | 25.87 |
| VAR-B54 | 41.9 | 42.98 | 54.79 | 64.69 | 27.25 | 27.63 | 50.55 | 51.82 | 33.14 | 35.06 | 47.9 | 21.3 | 50.73 | 47.03 | 37.04 | 35.02 |
| VAR-B74 | 18.31 | 19.67 | 18.5 | 36.45 | 14.63 | 9.84 | 24.14 | 26.96 | 15.74 | 13.48 | 26.34 | 8.29 | 10.11 | 15.58 | 18.98 | 12.4 |
| VAR-B87 | 31.11 | 30.01 | 54.19 | 28.27 | 13.15 | 28.54 | 30.87 | 30.98 | 29.47 | 35.51 | 67.62 | 27.49 | 16.53 | 33.56 | 24.58 | 26.22 |
| VAR-B61 | 16.22 | 17.31 | 23.2 | 17.81 | 9.95 | 19.35 | 23.46 | 19.65 | 16.94 | 12.65 | 13.65 | 13.48 | 11.63 | 18.46 | 9.54 | 16.8 |
| VAR-B116 | 13.39 | 18.5 | 11.92 | 22.24 | 5.24 | 12.7 | 19.31 | 18.45 | 13.25 | 17.11 | 2.42 | 10.82 | 14.41 | 24.2 | 19.12 | 21.38 |
| VAR-B100 | 22.35 | 28.52 | 18.86 | 37.94 | 16.17 | 17.72 | 36.64 | 31.52 | 26.05 | 24.09 | 30.57 | 24.09 | 20.27 | 49.99 | 11.93 | 21.08 |
| VAR-B80 | 14.9 | 18.21 | 15.91 | 23.83 | 8.31 | 14.79 | 12.5 | 16.89 | 7.83 | 13.81 | 27.7 | 19.72 | 26 | 26.44 | 21.71 | 17.08 |
| VAR-B89 | 27.05 | 18.9 | 35.73 | 19.18 | 14 | 20.82 | 32.21 | 18.95 | 23.38 | 23.91 | 43.42 | 18.9 | 23.03 | 11.71 | 26.64 | 14.95 |
| VAR-B102 | 4.75 | 7.98 | 1.95 | 13.5 | 3.22 | 3.79 | 0.45 | 11.36 | 7.78 | 5.59 | 0 | 0 | 10.46 | 4.19 | 10.03 | 8 |
| VAR-B122 | 3.01 | 2.73 | 2.18 | 2.05 | 2.96 | 1.38 | 6.27 | 4.42 | 1.69 | 2.91 | 1.1 | 0.68 | 3.05 | 4.22 | 1.95 | 2.51 |
| VAR-B75 | 5.22 | 6.29 | 6.92 | 12.33 | 2.92 | 3.15 | 6.76 | 5.24 | 5.94 | 8.63 | 3.41 | 0 | 6.69 | 5.89 | 3.38 | 3.9 |
| VAR-B96 | 2.76 | 2.75 | 4.34 | 2.09 | 1.28 | 4.08 | 3.58 | 4.18 | 3.05 | 0.96 | 2.76 | 2.41 | 4.26 | 1.36 | 0.95 | 2.76 |
| VAR-B67 | 9.64 | 10.69 | 12.72 | 10.68 | 5.82 | 8.54 | 12.26 | 14.46 | 6.99 | 7.15 | 18.4 | 14.52 | 15.86 | 13.05 | 5.16 | 9.16 |
| VAR-B57 | 6.25 | 7.12 | 6.49 | 7.66 | 6.29 | 6.35 | 8.35 | 5.61 | 5.45 | 6.65 | 3.64 | 4.27 | 7.13 | 13.28 | 4.67 | 7.85 |
| VAR-B59 | 3.25 | 3.74 | 0.53 | 4.34 | 3.73 | 2.48 | 1.71 | 6.89 | 5.25 | 2.15 | 1.49 | 0.18 | 9.12 | 5.81 | 3.47 | 1.99 |
| VAR-B35 | 2.16 | 0.52 | 0 | 0 | 0.13 | 0 | 5.91 | 0 | 0.59 | 3.35 | 20.05 | 0 | 0 | 0 | 0 | 0 |
| VAR-B110 | 0.9 | 0 | 0.76 | 0 | 0.02 | 0 | 0 | 0 | 0 | 0 | 10.53 | 0 | 2.78 | 0 | 0.52 | 0 |
| VAR-B93 | 1.64 | 2.5 | 1.39 | 2.09 | 1.29 | 1.4 | 0.97 | 1.56 | 2.32 | 1.35 | 1.4 | 5.71 | 3.61 | 8.75 | 1.64 | 2.63 |
| VAR-B76 | 0.23 | 1.16 | 0 | 1.18 | 1.26 | 0.21 | 0 | 1.27 | 0 | 0.04 | 0 | 0 | 0 | 0 | 0 | 3.89 |
| VAR-B119 | 1.82 | 1.89 | 5.62 | 4.35 | 1.5 | 2.2 | 1.35 | 1.11 | 0.8 | 0.96 | 0.11 | 2.03 | 0.54 | 0.52 | 0.71 | 1.23 |
| VAR-B82 | 3.33 | 3.87 | 2.46 | 3.63 | 3.61 | 3.76 | 3.02 | 4.2 | 3.25 | 1.94 | 6.53 | 3.03 | 2.24 | 6.84 | 3.97 | 4.59 |
| VAR-B83 | 2.59 | 3.31 | 2.49 | 6.21 | 2.42 | 3.03 | 0.43 | 0.41 | 3.21 | 3.7 | 4.94 | 0.61 | 4.74 | 8.42 | 3.2 | 2.27 |
| VAR-B108 | 0.27 | 1.72 | 0.01 | 4.88 | 0.12 | 1.17 | 0.92 | 0.09 | 0.01 | 2.48 | 1.16 | 3.8 | 0.04 | 0.02 | 0.08 | 0.17 |
| VAR-B71 | 2.82 | 2.6 | 3.8 | 1.29 | 2.04 | 1.37 | 1.79 | 0.57 | 4.11 | 3.74 | 1.96 | 7.78 | 3.99 | 5.95 | 2.35 | 3.76 |
| VAR-B90 | 1.69 | 1.79 | 1.25 | 0.68 | 0.74 | 0.49 | 3.7 | 1.88 | 1.22 | 1.88 | 3.5 | 9.98 | 0.83 | 0.32 | 1.22 | 2.22 |
| VAR-B123 | 0.91 | 1.15 | 1.1 | 0 | 0.37 | 0.45 | 1.85 | 0 | 0.62 | 0.9 | 0.01 | 12.37 | 0.24 | 1.14 | 1.01 | 1.4 |
| VAR-B44 | 0.73 | 0.41 | 0.15 | 0 | 0.42 | 0 | 1.15 | 0 | 1.9 | 2.25 | 0 | 0 | 0.4 | 0 | 0.43 | 0.39 |
| VAR-B86 | 1.01 | 1.44 | 0.68 | 0.02 | 2.72 | 2.98 | 0.7 | 0.25 | 0.31 | 4.96 | 0.14 | 0 | 2.18 | 0 | 0.32 | 0.74 |
| VAR-B81 | 0.99 | 1.92 | 0.93 | 0.79 | 0.76 | 1.34 | 0.89 | 1.46 | 0.61 | 2.4 | 3.32 | 0.01 | 1.17 | 8.14 | 1.06 | 1.81 |
| VAR-B43 | 0.94 | 1.26 | 1.36 | 1.78 | 0.18 | 0 | 2.8 | 1.68 | 0.14 | 0.82 | 0 | 3.81 | 0.35 | 2.22 | 0.42 | 0.58 |
| VAR-B41 | 0.8 | 0.71 | 0.22 | 0.15 | 0.64 | 0.82 | 1.77 | 0.02 | 0.6 | 1.17 | 0 | 1.58 | 0.48 | 1.26 | 0.99 | 1.17 |
| VAR-B88 | 0.99 | 1.04 | 0.57 | 0.83 | 0.88 | 0.76 | 0.12 | 1.12 | 4.12 | 0.78 | 0 | 0.21 | 0.2 | 1.21 | 0.26 | 1.87 |
| VAR-B127 | 0.82 | 0.93 | 1.77 | 0.01 | 0 | 0.84 | 1.67 | 0.08 | 0.69 | 2.11 | 0 | 0 | 0 | 5.73 | 0.4 | 0.25 |
| VAR-B85 | 1.13 | 1.18 | 0.46 | 1.84 | 1.72 | 0.97 | 1.6 | 0.84 | 0.93 | 1.62 | 0.75 | 0 | 1.26 | 0.01 | 0.93 | 1.55 |
| VAR-B51 | 0.38 | 0.16 | 0 | 0 | 0.64 | 0.11 | 0 | 0 | 0.63 | 0.85 | 1.96 | 0 | 0 | 0 | 0.43 | 0.05 |
| VAR-B99 | 0.7 | 2.4 | 0.51 | 0.35 | 0.83 | 0.1 | 0.47 | 11.07 | 0.66 | 0.04 | 0.75 | 0 | 1.02 | 0 | 0.93 | 0 |
| VAR-B101 | 0.61 | 0.6 | 1.17 | 0.72 | 0.64 | 0.34 | 0 | 0 | 0.42 | 0.84 | 1.16 | 0 | 0.89 | 1.13 | 0.58 | 1.23 |
| VAR-B107 | 1.01 | 0.59 | 0.77 | 2.18 | 0.63 | 0.59 | 0.5 | 0 | 0.3 | 0.13 | 7.69 | 0 | 1.76 | 0 | 0.81 | 0.4 |
| VAR-B5 | 0.25 | 0.39 | 0.22 | 0 | 0.13 | 0.6 | 0 | 0.75 | 0.56 | 0 | 1.44 | 0.42 | 0.24 | 0 | 0.11 | 0.69 |
| VAR-B33 | 1 | 0.66 | 0.61 | 1.43 | 0.52 | 0.77 | 1.99 | 0.19 | 1.19 | 0.71 | 0.37 | 0 | 0.6 | 0.75 | 0.97 | 0.41 |
| VAR-B3 | 0.74 | 0.33 | 1.24 | 0 | 0.55 | 0.19 | 0 | 0 | 0 | 0.02 | 0 | 0 | 2.7 | 3.95 | 1.3 | 0.07 |
| VAR-B25 | 0.62 | 0.8 | 0.67 | 0.7 | 0.16 | 1.21 | 0.84 | 0 | 0.43 | 2.19 | 1.79 | 0.02 | 0.59 | 0 | 0.65 | 0.79 |
| VAR-B49 | 0.42 | 0.37 | 0 | 0.24 | 0.2 | 0 | 0 | 0.92 | 2.45 | 0.1 | 0 | 0 | 0 | 0 | 0 | 0.74 |
| VAR-B68 | 0.85 | 1.31 | 0.56 | 0.12 | 0.88 | 1.31 | 0.64 | 2.54 | 0.63 | 2.02 | 0.78 | 1.03 | 1.27 | 0.83 | 1.39 | 0.71 |
| VAR-B40 | 0.71 | 0.88 | 1.76 | 0.64 | 0.37 | 0.72 | 0.49 | 0.97 | 0.88 | 1.95 | 0 | 0 | 1.42 | 0 | 0 | 0.85 |
| VAR-B97 | 0.93 | 1.74 | 2.12 | 0.49 | 0.69 | 0.4 | 0 | 6.24 | 0 | 0.1 | 5.92 | 2.03 | 1.14 | 0.94 | 0.49 | 0.55 |
| VAR-B10 | 0.95 | 0.71 | 0.65 | 1.1 | 1.32 | 0.47 | 0.61 | 0.18 | 1.57 | 0.77 | 0 | 0.62 | 0.39 | 0 | 1.16 | 1.5 |
| VAR-B105 | 0.78 | 0.92 | 0.61 | 0.32 | 0.64 | 1.64 | 0.6 | 0.75 | 0.68 | 1.01 | 0 | 0 | 4.32 | 0.77 | 0.15 | 1.36 |
| VAR-B114 | 2.49 | 1.92 | 1.67 | 2 | 1.26 | 2.64 | 4.74 | 0.41 | 2.89 | 3.26 | 0.82 | 2.11 | 2.29 | 2.54 | 2.25 | 1.45 |
| VAR-B69 | 0.75 | 1.96 | 0.82 | 0.04 | 0.86 | 0.51 | 1.52 | 0.39 | 0.32 | 4.16 | 1.58 | 0 | 0.01 | 12.62 | 0.18 | 1.48 |
| VAR-B14 | 0.97 | 0.24 | 0.37 | 0.61 | 0.97 | 0.77 | 0.49 | 0 | 0.32 | 0.02 | 11.5 | 0 | 0 | 0 | 0.38 | 0 |
| VAR-B70 | 0.92 | 1.17 | 0.04 | 0.66 | 0 | 0.19 | 4.58 | 3.33 | 0.1 | 0.63 | 0 | 0 | 0 | 0.55 | 0.09 | 1.1 |
| VAR-B55 | 1.03 | 0.28 | 0.25 | 0 | 0.36 | 0 | 2.57 | 0.01 | 0.13 | 0.16 | 3.55 | 0 | 0.6 | 1.69 | 1.15 | 0.81 |
| VAR-B78 | 1.2 | 0.33 | 0.15 | 0 | 1.36 | 0.09 | 0 | 0 | 0.81 | 0.9 | 6.2 | 0 | 2.29 | 0.92 | 2.03 | 0.65 |
| VAR-B50 | 0.88 | 0.53 | 0.71 | 0.65 | 0.29 | 0.91 | 0.93 | 0.27 | 0.16 | 0.56 | 0 | 0.08 | 1.5 | 0 | 2.22 | 0.7 |
| VAR-B53 | 0.6 | 0.49 | 0.14 | 0.07 | 0.11 | 2.46 | 0 | 0 | 2.91 | 0.44 | 0 | 0 | 0.2 | 0 | 0.47 | 0.04 |
| VAR-B77 | 0.57 | 0.24 | 0.01 | 0.01 | 0 | 0.25 | 0 | 0.68 | 2.98 | 0.31 | 0 | 0.07 | 0.6 | 0.03 | 0.32 | 0.01 |
| VAR-B109 | 0.46 | 0.92 | 0.65 | 1.76 | 0 | 0.58 | 0 | 1.64 | 1.17 | 0.09 | 0.77 | 0 | 0 | 0.76 | 0.74 | 0.53 |
| VAR-B112 | 0.89 | 0.67 | 0.77 | 0.03 | 0.85 | 0.54 | 0.29 | 0.23 | 1.01 | 0.68 | 0 | 5.5 | 1.21 | 0 | 1.68 | 0.86 |
| VAR-B124 | 1 | 1.21 | 2.43 | 1.26 | 0.82 | 0.53 | 0.45 | 2.15 | 0.75 | 1.38 | 2.7 | 1.39 | 0.46 | 0 | 0.37 | 0.93 |
| VAR-B46 | 0.42 | 0.44 | 1.66 | 0.36 | 0.16 | 0.87 | 0 | 1.03 | 0.3 | 0.11 | 0 | 0 | 0 | 0 | 0.27 | 0 |
| VAR-B28 | 1.28 | 1.17 | 0.64 | 0.81 | 1.29 | 2.04 | 1.61 | 0.11 | 0.47 | 1.71 | 4.99 | 1.42 | 2.39 | 1.77 | 0.88 | 1.2 |
| VAR-B17 | 0.56 | 1.46 | 0.14 | 1.02 | 0.42 | 0.77 | 0.77 | 1.26 | 0.89 | 4.01 | 0 | 0 | 0.43 | 0.98 | 0.8 | 1.19 |
| VAR-B47 | 0.34 | 0.44 | 0.04 | 0.12 | 1.29 | 0.56 | 0 | 0.68 | 0.16 | 0.34 | 0 | 0 | 0.59 | 0.02 | 0.18 | 0.8 |
| VAR-B62 | 0.89 | 0.93 | 1.04 | 0.29 | 1.12 | 0.77 | 0.06 | 1.31 | 0.7 | 0.65 | 1.67 | 0 | 0.67 | 0.42 | 1.44 | 2.09 |
| VAR-B2 | 0.84 | 0.73 | 1.86 | 0 | 0.18 | 1.72 | 0.01 | 1.52 | 2.31 | 0.77 | 0 | 0 | 0.01 | 0.17 | 0.65 | 0 |
| VAR-B30 | 0.26 | 0.14 | 0.66 | 0 | 0.08 | 0.39 | 0.37 | 0 | 0.06 | 0.31 | 0 | 0 | 0 | 0 | 0.27 | 0.18 |
| VAR-B103 | 0.49 | 0.23 | 1.34 | 0 | 0 | 0.55 | 0 | 0 | 1.6 | 0 | 0 | 0 | 0 | 0.16 | 0.01 | 0.8 |
| VAR-B72 | 0.37 | 0.62 | 0 | 0 | 0.4 | 0.85 | 0 | 0 | 0.41 | 1.12 | 0 | 0 | 0.02 | 0 | 1.34 | 1.87 |
| VAR-B23 | 0.12 | 0.54 | 0.18 | 0.11 | 0 | 0.07 | 0.29 | 1.86 | 0 | 0.46 | 0 | 0 | 0.17 | 0.19 | 0.14 | 0.18 |
| VAR-B95 | 3.71 | 2.86 | 4.02 | 2.1 | 2.26 | 2.94 | 2.79 | 1.4 | 3.28 | 5.18 | 17 | 1.81 | 4.39 | 6.21 | 2.6 | 2.15 |
| VAR-B73 | 0.41 | 0.97 | 2.11 | 1.02 | 0 | 1.83 | 0 | 0.81 | 0.24 | 0.44 | 0 | 0 | 0 | 1.16 | 0 | 0.94 |
| VAR-B79 | 0.21 | 1.21 | 0 | 3.89 | 0.6 | 0.05 | 0 | 0 | 0.27 | 0 | 0 | 0 | 0 | 0 | 0.35 | 2.96 |
| VAR-B115 | 1.35 | 1.1 | 0.55 | 0.27 | 1.11 | 1.55 | 3.89 | 1.25 | 0.51 | 0.17 | 0.44 | 6.4 | 0.01 | 0.12 | 1.15 | 1.02 |
| VAR-B98 | 1.14 | 1.37 | 0.28 | 1.44 | 0.64 | 1.42 | 1.33 | 2.3 | 0.27 | 0.5 | 8.06 | 1.86 | 3.56 | 0.36 | 0.3 | 1.13 |
| VAR-B125 | 0.8 | 0.57 | 0.9 | 2.94 | 0.68 | 0.02 | 0 | 0.02 | 0.98 | 0.1 | 0 | 0 | 3.96 | 0 | 0.47 | 0 |
| VAR-B104 | 1.79 | 0.58 | 1.94 | 0 | 1.35 | 2.41 | 2.75 | 0 | 1.8 | 0.7 | 1.7 | 0 | 2.81 | 0.17 | 0.64 | 0.36 |
| VAR-B31 | 0.59 | 0.23 | 0.33 | 0.07 | 0.44 | 0.38 | 0 | 0.23 | 0.45 | 0.61 | 0.06 | 0 | 0.01 | 0 | 2.17 | 0.07 |
| VAR-B24 | 0.87 | 0.83 | 0.38 | 0.6 | 0.64 | 0.2 | 1.86 | 1.13 | 1.06 | 1.39 | 0 | 1.73 | 0.28 | 0 | 0.81 | 0.88 |
| VAR-B39 | 0.16 | 0.06 | 0 | 0 | 0.25 | 0 | 0 | 0.01 | 0 | 0.02 | 0 | 0 | 0 | 0.3 | 0.66 | 0.19 |
| VAR-B8 | 1.12 | 0.53 | 0.53 | 1.91 | 0.11 | 0.39 | 1.04 | 0.01 | 1.46 | 0.28 | 8.29 | 0 | 2.73 | 0.11 | 0.05 | 0.33 |
| VAR-B11 | 0.87 | 0.84 | 0.65 | 0.01 | 1.3 | 4.13 | 0.89 | 0 | 0.66 | 0.46 | 0.65 | 0 | 1.32 | 0 | 0.7 | 0.52 |
| VAR-B32 | 0.99 | 0.63 | 1.19 | 0.79 | 0.54 | 0.67 | 1.48 | 0 | 0.97 | 1.06 | 0.14 | 0.05 | 0.33 | 0.14 | 1.22 | 1.2 |
| VAR-B6 | 1.37 | 0.83 | 2.61 | 0.31 | 0.65 | 0.75 | 1.28 | 0.29 | 1.87 | 2.21 | 1.75 | 1.47 | 0.02 | 0.84 | 0.96 | 0.66 |
| VAR-B121 | 0.97 | 0.78 | 0.78 | 0.8 | 0.65 | 0.77 | 1.75 | 0.76 | 0.26 | 1.41 | 0.74 | 0.96 | 1.55 | 0.03 | 1.06 | 0.51 |
| VAR-B9 | 1 | 0.26 | 1.28 | 0.1 | 2.02 | 0.17 | 0.06 | 0.07 | 0.6 | 0.45 | 1.9 | 1.84 | 1.99 | 0.01 | 0.47 | 0.19 |
| VAR-B65 | 0.96 | 0.55 | 0.37 | 1.29 | 0.17 | 0.48 | 3.63 | 0.23 | 0.41 | 0.23 | 0.02 | 1.21 | 0.3 | 0.75 | 0.42 | 0.22 |
| VAR-B21 | 1.28 | 0.72 | 0.32 | 0.03 | 1.61 | 0.15 | 2.93 | 2.15 | 0.57 | 0.33 | 0 | 0.29 | 2.18 | 0.08 | 0.7 | 1.03 |
| VAR-B58 | 0.76 | 0.89 | 0.8 | 0.56 | 0.88 | 1.03 | 0.46 | 0.39 | 1.58 | 0.18 | 0.37 | 3.98 | 0.17 | 0.24 | 0.56 | 1.75 |
| VAR-B34 | 0.52 | 1.24 | 0.5 | 3.57 | 0.77 | 0.61 | 0.12 | 0.93 | 1.23 | 0.43 | 0 | 0.41 | 0 | 2.28 | 0.42 | 0.17 |
| VAR-B120 | 1.01 | 0.85 | 1.35 | 0.87 | 0.53 | 0.45 | 1.27 | 0.87 | 1.39 | 1.25 | 0.49 | 1 | 1.44 | 0.21 | 0.51 | 1.08 |
| VAR-B118 | 0.73 | 1.31 | 0.3 | 1.23 | 0.47 | 2.11 | 0.39 | 1.31 | 0.01 | 0.84 | 0 | 0 | 4.41 | 0.01 | 1.16 | 2.02 |
| VAR-B27 | 0.59 | 0.55 | 0.17 | 0.18 | 1.38 | 0.37 | 0.77 | 0.22 | 0.55 | 0.58 | 0.01 | 2.82 | 0.22 | 0 | 0.37 | 1.08 |
| VAR-B29 | 0.45 | 0.47 | 0.61 | 0.85 | 0.58 | 0.5 | 0.32 | 0.11 | 0.34 | 0.24 | 0.88 | 1.64 | 0.29 | 0.36 | 0.38 | 0.4 |
| VAR-B4 | 0.34 | 0.67 | 0 | 0.01 | 0.31 | 0.05 | 0 | 0.8 | 1.22 | 0.32 | 0 | 0 | 0 | 1.58 | 0.51 | 2.02 |
| VAR-B7 | 1.69 | 1.12 | 3.28 | 2.53 | 1.95 | 0.24 | 1.88 | 0.13 | 0.16 | 3.39 | 0 | 0 | 3.42 | 0.08 | 0.7 | 0.35 |
| VAR-B12 | 1.12 | 0.81 | 0.43 | 0.85 | 2.58 | 0.47 | 0.01 | 0.83 | 1.15 | 0.65 | 0 | 0 | 1.58 | 10.9 | 1.61 | 1.46 |
| VAR-B13 | 0.55 | 0.07 | 0.46 | 0 | 0.32 | 0 | 0.49 | 0 | 0.98 | 0.03 | 0 | 0 | 1.32 | 0 | 0.39 | 0.4 |
| VAR-B15 | 1.58 | 2.11 | 0.99 | 6.46 | 1.62 | 1.69 | 1.75 | 0.47 | 1.73 | 0.28 | 0 | 0 | 6.08 | 0.24 | 0.42 | 2.88 |
| VAR-B16 | 0.97 | 0.96 | 0.61 | 0.66 | 3 | 0 | 1.41 | 3.2 | 0.1 | 0.27 | 0 | 0 | 0 | 0 | 0.18 | 0.76 |
| VAR-B18 | 1.09 | 0.19 | 0 | 0 | 5.84 | 0.36 | 0 | 0 | 0.01 | 0.54 | 0 | 0 | 0 | 0 | 0.25 | 0.31 |
| VAR-B19 | 1.07 | 0.98 | 1.61 | 0 | 1.87 | 1.59 | 0 | 0 | 0 | 0 | 0 | 14.06 | 0 | 0 | 2.56 | 0 |
| VAR-B20 | 1.77 | 1.2 | 0.85 | 3.09 | 1.76 | 0.58 | 0.81 | 0.55 | 0.97 | 0.02 | 5.76 | 0 | 0.3 | 4.91 | 4.05 | 0.36 |
| VAR-B22 | 0.63 | 1.3 | 0 | 2.65 | 0.34 | 0.22 | 1.66 | 0 | 0.98 | 0.58 | 0 | 0 | 0.97 | 4.13 | 0.14 | 2.35 |
| VAR-B26 | 1.1 | 1.3 | 1.73 | 1.94 | 0.9 | 0.63 | 0 | 0 | 0.39 | 0.01 | 0 | 10.33 | 4.51 | 0 | 1.39 | 1.86 |
| VAR-B36 | 0.16 | 0.24 | 0 | 0 | 0 | 0 | 0.02 | 0 | 0.93 | 1.58 | 0 | 0 | 0 | 0 | 0.04 | 0 |
| VAR-B37 | 0.85 | 1.8 | 0 | 0.26 | 1.19 | 0.77 | 0.81 | 0 | 0.01 | 7.43 | 0 | 8.3 | 2.59 | 0 | 1.68 | 0.3 |
| VAR-B38 | 0.49 | 0.13 | 0.14 | 0 | 1.2 | 0.18 | 0.61 | 0 | 0 | 0.64 | 2.75 | 0 | 0 | 0 | 0.05 | 0 |
| VAR-B42 | 0.77 | 0.99 | 0.47 | 3.37 | 0.98 | 0.69 | 0.35 | 0 | 0.86 | 0.64 | 5.87 | 1.77 | 0 | 0.06 | 0.24 | 0.38 |
| VAR-B45 | 0.25 | 0.62 | 0 | 0.36 | 0.04 | 0.11 | 0 | 0 | 0.34 | 1.99 | 0 | 3.69 | 0 | 0 | 1.08 | 0.22 |
| VAR-B48 | 1.15 | 1.35 | 0.24 | 1.29 | 1.36 | 1.03 | 1.35 | 0.66 | 1.1 | 3.48 | 2.37 | 0.03 | 1.7 | 0.49 | 1.15 | 1.41 |
| VAR-B52 | 0.18 | 0.44 | 0.03 | 0.9 | 0.17 | 0.08 | 0 | 0 | 0.8 | 1.54 | 0 | 0 | 0 | 0 | 0.11 | 0.12 |
| VAR-B56 | 1.04 | 1.46 | 1.57 | 0.53 | 0.36 | 0.94 | 0.01 | 3.87 | 1.32 | 0.62 | 0.02 | 3.25 | 2.95 | 0.01 | 1.58 | 0.84 |
| VAR-B60 | 1.5 | 0.73 | 3.11 | 0.08 | 0 | 3.48 | 4.86 | 0.01 | 0.05 | 0.23 | 0 | 0 | 0 | 0 | 0.01 | 0.65 |
| VAR-B63 | 3.19 | 1.18 | 2.62 | 0.71 | 2.92 | 0.59 | 8.08 | 2.76 | 0.68 | 0.83 | 0 | 0.15 | 3.47 | 1.22 | 1.6 | 0.92 |
| VAR-B64 | 0.55 | 0.18 | 0 | 0 | 0 | 0 | 0 | 0 | 0 | 0 | 0 | 0 | 7.66 | 0 | 0 | 1.09 |
| VAR-B66 | 0.37 | 1.03 | 0.8 | 0.02 | 0.88 | 0.05 | 0 | 3.97 | 0.33 | 0.29 | 0 | 0 | 0 | 1.42 | 0.08 | 0.26 |
| VAR-B84 | 0.56 | 1.62 | 1.9 | 0 | 0.19 | 0.04 | 0 | 0 | 0.83 | 3.16 | 0 | 18.49 | 0.66 | 0 | 0.07 | 1.09 |
| VAR-B91 | 0.23 | 0 | 0.56 | 0 | 0 | 0 | 0 | 0 | 0.54 | 0 | 0.03 | 0 | 0 | 0 | 0.26 | 0 |
| VAR-B92 | 0.3 | 1.41 | 0.54 | 3.65 | 0.84 | 4.05 | 0 | 0 | 0.05 | 0.44 | 0 | 0 | 0.48 | 0 | 0.04 | 0 |
| VAR-B94 | 1.05 | 0.29 | 0.44 | 1.19 | 4.27 | 0 | 0 | 0.11 | 0.11 | 0 | 0 | 0 | 0 | 0 | 1.04 | 0.27 |
| VAR-B106 | 0.47 | 1.13 | 0 | 0.19 | 0.99 | 0.87 | 0.8 | 2.15 | 0.53 | 1.08 | 0 | 0 | 0 | 1.07 | 0.33 | 1.58 |
| VAR-B111 | 1.37 | 0.47 | 0 | 0.54 | 0 | 0.57 | 4.69 | 0 | 0.88 | 0.35 | 0 | 0 | 1.13 | 0.01 | 1.37 | 1.34 |
| VAR-B113 | 1.4 | 1.05 | 0 | 1.68 | 0.43 | 1 | 0 | 0 | 6.18 | 2.23 | 0.05 | 2.05 | 0 | 0.45 | 1.98 | 0.56 |
| VAR-B117 | 1.62 | 0.82 | 1.35 | 1.07 | 3.85 | 1.05 | 2.5 | 0.06 | 0.2 | 1.31 | 0 | 0 | 0 | 1.45 | 1 | 0.83 |
| VAR-B126 | 0.87 | 0.97 | 0 | 0.1 | 2.4 | 3.67 | 0 | 0.33 | 2 | 1.71 | 0 | 0 | 0 | 0 | 0.74 | 0.09 |

TABLE 18
Fold change in biodistribution of virus particles comprising the indicated variant capsid polypeptide,
relative to the biodistribution of virus particles comprising WT AAV9 for non-brain regions. CBh indicates
measurements from virus particles comprising genomes with the cap gene under the control of the CBh
promoter. hSyn indicates virus particles comprising genomes with the cap gene under the control of
the hSyn promoter. Values of “0” indicate variant was not detected in the indicated sample.

| | ORGAN | | | | | | | | | |
|---|---|---|---|---|---|---|---|---|---|---|
| | Dorsal Root | Dorsal Root | | | Skeletal | Skeletal | Spinal | Spinal | | |
| | Ganglion | Ganglion | Liver | Liver | Muscle | Muscle | Cord | Cord | Spleen | Spleen |
| | TISSUE | | | | | | | | | |
| | Aggre- | Aggre- | Aggre- | Aggre- | Aggre- | Aggre- | Aggre- | Aggre- | Aggre- | Aggre- |
| | gated | gated | gated | gated | gated | gated | gated | gated | gated | gated |
| | PROMOTER | | | | | | | | | |
| VAR-# | CBh | hSyn | CBh | hSyn | CBh | hSyn | CBh | hSyn | CBh | hSyn |
| VAR-B1 | 5.37 | 8.11 | 0.32 | 0.3 | 4.22 | 4.62 | 18.01 | 11.83 | 0.85 | 0.9 |
| VAR-B54 | 3.94 | 6.71 | 0.37 | 0.32 | 5.07 | 5.37 | 22.73 | 21.66 | 0.7 | 0.85 |
| VAR-B74 | 10.37 | 10.08 | 0.27 | 0.26 | 3.33 | 5.22 | 16.29 | 15.2 | 0.76 | 0.73 |
| VAR-B87 | 5.85 | 2.93 | 0.68 | 0.6 | 3.57 | 2.9 | 24.18 | 18.85 | 0.84 | 0.65 |
| VAR-B61 | 6.39 | 8.33 | 0.42 | 0.31 | 4.57 | 1.14 | 19.96 | 1.39 | 0.72 | 0.56 |
| VAR-B116 | 3.14 | 2.36 | 0.65 | 0.69 | 2.24 | 2.51 | 11.11 | 5.18 | 1.22 | 1.09 |
| VAR-B100 | 1.25 | 1.31 | 0.68 | 0.77 | 2.72 | 2.42 | 14.08 | 8.55 | 0.56 | 1.19 |
| VAR-B80 | 3.83 | 13.9 | 0.44 | 0.39 | 2.56 | 4.26 | 20.78 | 15.31 | 0.81 | 0.67 |
| VAR-B89 | 3.4 | 1.15 | 0.71 | 0.69 | 1.88 | 5.63 | 11.21 | 19.88 | 1.68 | 0.86 |
| VAR-B102 | 27.04 | 3.8 | 0.12 | 0.15 | 1.98 | 1.11 | 17.77 | 4.6 | 0.32 | 0.11 |
| VAR-B122 | 1.52 | 0.71 | 0.33 | 0.35 | 0.5 | 0.89 | 4.98 | 1.14 | 0.74 | 1.01 |
| VAR-B75 | 1.3 | 0.12 | 0.51 | 0.46 | 0.79 | 1.7 | 5.53 | 0.92 | 1.13 | 1.07 |
| VAR-B96 | 1.83 | 0.6 | 0.23 | 0.23 | 0.88 | 0.8 | 2.8 | 0.99 | 0.71 | 0.6 |
| VAR-B67 | 1.01 | 3.17 | 0.71 | 0.71 | 1.73 | 2.16 | 4.44 | 7.29 | 0.94 | 0.86 |
| VAR-B57 | 2.4 | 1.71 | 0.61 | 0.61 | 1.08 | 2.41 | 2.99 | 3.31 | 0.46 | 0.66 |
| VAR-B59 | 1.73 | 1.6 | 0.49 | 0.5 | 0.54 | 1.99 | 0.88 | 3.25 | 0.51 | 0.8 |
| VAR-B35 | 0.08 | 0 | 0.23 | 0.37 | 0 | 0 | 0 | 0 | 0.44 | 1.11 |
| VAR-B110 | 1.98 | 0 | 0.64 | 0.67 | 1.74 | 2.05 | 0.09 | 0.31 | 1.14 | 0.77 |
| VAR-B93 | 0.95 | 1.45 | 0.89 | 0.78 | 1.37 | 0.46 | 0.52 | 0.48 | 0.81 | 0.73 |
| VAR-B76 | 0.06 | 2.24 | 0.63 | 0.78 | 0.4 | 0.05 | 0.52 | 0.26 | 0.97 | 1.14 |
| VAR-B119 | 1.39 | 0.83 | 0.83 | 0.79 | 1.15 | 0.78 | 0.93 | 1.3 | 0.59 | 0.6 |
| VAR-B82 | 1.35 | 0.16 | 0.84 | 0.83 | 0.34 | 1.35 | 0.65 | 0.55 | 0.73 | 1.05 |
| VAR-B83 | 0.37 | 0.81 | 0.95 | 0.97 | 2.71 | 1.87 | 1.19 | 0.55 | 0.8 | 0.84 |
| VAR-B108 | 0.17 | 0.06 | 0.34 | 0.45 | 0 | 0.49 | 11.04 | 0.9 | 1.52 | 1.17 |
| VAR-B71 | 0.62 | 1.01 | 0.93 | 1.02 | 1.07 | 2.19 | 2.36 | 1.7 | 1.03 | 1.06 |
| VAR-B90 | 0.59 | 0.8 | 0.65 | 0.76 | 1.31 | 0 | 0.57 | 0.04 | 1 | 1.15 |
| VAR-B123 | 0.1 | 1.09 | 0.65 | 0.63 | 0.74 | 0.96 | 1.77 | 1.47 | 0.79 | 0.74 |
| VAR-B44 | 0.38 | 0 | 1 | 1.11 | 0.34 | 1.04 | 4.07 | 0.07 | 0.65 | 0.86 |
| VAR-B86 | 2.4 | 0.99 | 1.01 | 1.06 | 2.3 | 0.38 | 0.39 | 0 | 1.03 | 0.99 |
| VAR-B81 | 0.73 | 1.76 | 0.73 | 0.67 | 0.75 | 1.88 | 2.33 | 0.04 | 0.92 | 0.62 |
| VAR-B43 | 0 | 1.03 | 0.73 | 0.86 | 2.04 | 0.36 | 0 | 1.44 | 0.77 | 1.26 |
| VAR-B41 | 0.75 | 0.14 | 0.61 | 0.67 | 0.74 | 1.48 | 0.55 | 0.69 | 0.96 | 1.05 |
| VAR-B88 | 0.79 | 1.11 | 0.38 | 0.35 | 0.73 | 1.21 | 1.71 | 0.54 | 0.54 | 0.76 |
| VAR-B127 | 0.06 | 0 | 0.75 | 0.78 | 0 | 0 | 5.7 | 0 | 1.16 | 1.3 |
| VAR-B85 | 0.42 | 0.71 | 0.92 | 1.04 | 1.29 | 0.74 | 0.67 | 7.03 | 0.78 | 1.09 |
| VAR-B51 | 0.24 | 2.24 | 0.84 | 0.78 | 1.29 | 0.62 | 0.18 | 0 | 1.23 | 0.5 |
| VAR-B99 | 2.53 | 0.34 | 0.62 | 0.73 | 1.2 | 0 | 0.04 | 0 | 0.84 | 1.37 |
| VAR-B101 | 1.07 | 0.09 | 0.98 | 0.91 | 0.39 | 0.96 | 0.33 | 0.48 | 1 | 0.94 |
| VAR-B107 | 0.6 | 0.43 | 0.45 | 0.45 | 0.74 | 0.44 | 0.04 | 0.62 | 0.84 | 0.95 |
| VAR-B5 | 1.28 | 0.13 | 0.32 | 0.36 | 0.52 | 0.74 | 0.09 | 0.03 | 0.57 | 0.84 |
| VAR-B33 | 1.09 | 0.7 | 0.5 | 0.44 | 0.63 | 0.98 | 1.37 | 2.06 | 1.15 | 1.52 |
| VAR-B3 | 2.26 | 0.86 | 0.96 | 1.03 | 1.06 | 0 | 0.33 | 0 | 1.18 | 0.75 |
| VAR-B25 | 0.43 | 0.45 | 0.36 | 0.39 | 2.38 | 0.46 | 0 | 0.68 | 1.08 | 1.07 |
| VAR-B49 | 0 | 0.26 | 0.24 | 0.22 | 0 | 0.78 | 0 | 0 | 0.55 | 1.16 |
| VAR-B68 | 0.94 | 0.29 | 1.03 | 0.94 | 1.3 | 1.28 | 0.12 | 0.09 | 0.81 | 1.01 |
| VAR-B40 | 0.23 | 1.21 | 0.81 | 0.66 | 1.54 | 0.01 | 0.85 | 0 | 0.69 | 0.67 |
| VAR-B97 | 0 | 1.02 | 0.79 | 0.88 | 2.17 | 0.61 | 0.64 | 0.31 | 0.57 | 1.18 |
| VAR-B10 | 1.52 | 0.99 | 0.88 | 0.96 | 0.55 | 1.19 | 0.97 | 0.86 | 0.94 | 0.78 |
| VAR-B105 | 2.94 | 0.75 | 0.43 | 0.44 | 0.46 | 0.27 | 0.37 | 0.41 | 0.88 | 0.82 |
| VAR-B114 | 1.49 | 1.3 | 0.85 | 0.73 | 1.02 | 1.48 | 0.44 | 1.22 | 1.22 | 0.98 |
| VAR-B69 | 0.84 | 1.77 | 0.79 | 0.89 | 1.53 | 0.75 | 4.14 | 0.32 | 0.98 | 1.06 |
| VAR-B14 | 0.09 | 1.3 | 0.97 | 0.99 | 0.01 | 0 | 1.17 | 0.22 | 0.88 | 0.85 |
| VAR-B70 | 0.5 | 0.31 | 1.07 | 1.03 | 0.23 | 0.78 | 0.01 | 2.31 | 0.96 | 1.28 |
| VAR-B55 | 0.53 | 0.1 | 0.91 | 0.73 | 0.39 | 0.41 | 0.58 | 0 | 1.23 | 0.85 |
| VAR-B78 | 1.27 | 0.7 | 1.17 | 1.06 | 0 | 0.59 | 0.13 | 0.29 | 0.88 | 2.23 |
| VAR-B50 | 0.66 | 1.77 | 0.54 | 0.53 | 1.67 | 0 | 0.47 | 0 | 0.55 | 1.01 |
| VAR-B53 | 0.79 | 0.83 | 0.6 | 0.57 | 1.24 | 1.32 | 0.01 | 7.93 | 0.52 | 0.63 |
| VAR-B77 | 0.48 | 0.54 | 0.6 | 0.52 | 1.7 | 0.96 | 1.24 | 0.03 | 1.17 | 0.88 |
| VAR-B109 | 0.25 | 0.04 | 0.67 | 0.72 | 0.54 | 0.07 | 0.76 | 0.49 | 0.68 | 0.78 |
| VAR-B112 | 1.36 | 1.46 | 1.01 | 0.96 | 2.2 | 1.31 | 0.52 | 2.24 | 1.05 | 1.14 |
| VAR-B124 | 0.46 | 0.19 | 0.79 | 0.86 | 0.96 | 0.78 | 0.45 | 1.04 | 0.82 | 1.06 |
| VAR-B46 | 0.42 | 0 | 0.63 | 0.6 | 3.5 | 0.65 | 3.47 | 0 | 0.78 | 1.64 |
| VAR-B28 | 0.31 | 0.45 | 0.53 | 0.49 | 0.33 | 0.43 | 0.14 | 1.62 | 0.87 | 1.3 |
| VAR-B17 | 0.36 | 0.15 | 0.89 | 0.97 | 0.59 | 0.89 | 0.21 | 0.13 | 1.05 | 1.18 |
| VAR-B47 | 1.35 | 0.3 | 0.59 | 0.54 | 0.92 | 0.87 | 0.25 | 1.19 | 0.68 | 0.74 |
| VAR-B62 | 2.24 | 0.99 | 0.59 | 0.55 | 0.75 | 0.67 | 1.6 | 0.31 | 0.78 | 1.08 |
| VAR-B2 | 0.47 | 1.53 | 0.95 | 0.85 | 0.45 | 0.39 | 0.95 | 0.67 | 0.92 | 0.99 |
| VAR-B30 | 0.43 | 0.23 | 0.66 | 0.63 | 0 | 0.48 | 0 | 0.01 | 0.52 | 0.82 |
| VAR-B103 | 0.29 | 18.77 | 0.77 | 0.78 | 0.72 | 1.05 | 0.05 | 0 | 0.59 | 0.88 |
| VAR-B72 | 0 | 0.18 | 0.27 | 0.24 | 0.42 | 0.08 | 0.9 | 0 | 0.41 | 0.52 |
| VAR-B23 | 0.99 | 0.38 | 0.22 | 0.17 | 0.81 | 0.25 | 0 | 0.83 | 0.48 | 0.48 |
| VAR-B95 | 2.77 | 1.43 | 0.68 | 0.64 | 1.6 | 1.89 | 1.83 | 3.13 | 0.94 | 1.19 |
| VAR-B73 | 1.8 | 1.88 | 1.23 | 0.79 | 3.95 | 0.49 | 0 | 0.53 | 1 | 1.02 |
| VAR-B79 | 0.76 | 2.01 | 0.89 | 1 | 0.94 | 0.01 | 0 | 0 | 0.61 | 1.25 |
| VAR-B115 | 0.93 | 1.51 | 0.73 | 0.71 | 0 | 0.47 | 4.24 | 4.26 | 0.95 | 0.69 |
| VAR-B98 | 2.7 | 2.31 | 0.87 | 0.89 | 0.98 | 0.66 | 2.67 | 0.37 | 0.83 | 1.11 |
| VAR-B125 | 0.74 | 3.48 | 1.04 | 0.89 | 0.66 | 0.89 | 0.32 | 0.1 | 1.45 | 1.1 |
| VAR-B104 | 1.32 | 1.07 | 0.85 | 0.69 | 2.83 | 2.68 | 0.11 | 0.96 | 1.11 | 1.27 |
| VAR-B31 | 0.54 | 0.17 | 0.37 | 0.33 | 0.11 | 0.9 | 0.34 | 0.2 | 0.61 | 0.72 |
| VAR-B24 | 1.56 | 1.51 | 0.85 | 0.95 | 0.99 | 3.98 | 0.25 | 1.51 | 0.83 | 1.03 |
| VAR-B39 | 0 | 0.34 | 0.19 | 0.21 | 1.66 | 0.5 | 0 | 0 | 0.61 | 0.76 |
| VAR-B8 | 1.72 | 0.69 | 0.72 | 0.68 | 0.93 | 2.11 | 5.19 | 0.57 | 1.52 | 0.93 |
| VAR-B11 | 2.01 | 0.73 | 0.78 | 0.81 | 0 | 2.22 | 0.36 | 1.53 | 1.07 | 1.26 |
| VAR-B32 | 0.28 | 0.11 | 0.75 | 0.81 | 1.05 | 0.96 | 0.88 | 0.36 | 1.06 | 0.96 |
| VAR-B6 | 0.88 | 0.53 | 0.89 | 0.93 | 1.06 | 1.28 | 0.29 | 1.59 | 1.05 | 1.1 |
| VAR-B121 | 0.61 | 0.21 | 0.91 | 0.86 | 1.04 | 1.6 | 0.15 | 0.26 | 0.83 | 0.92 |
| VAR-B9 | 0.54 | 1.19 | 1.23 | 1.01 | 0.95 | 0.44 | 0.5 | 0.31 | 1.34 | 0.81 |
| VAR-B65 | 0.93 | 0.74 | 0.89 | 0.89 | 0.72 | 1.09 | 0.89 | 0 | 0.93 | 1.19 |
| VAR-B21 | 0.8 | 0.48 | 0.63 | 0.6 | 0.31 | 0.6 | 1.27 | 2.09 | 0.93 | 0.87 |
| VAR-B58 | 0.7 | 0.28 | 1.07 | 1.1 | 1.04 | 0.32 | 0.31 | 0.65 | 0.93 | 0.96 |
| VAR-B34 | 1.14 | 0.3 | 0.31 | 0.32 | 0.87 | 1.41 | 2.45 | 0.73 | 0.79 | 1.11 |
| VAR-B120 | 0.4 | 1 | 1.05 | 0.93 | 0.59 | 0.05 | 0.94 | 0.55 | 1.26 | 0.69 |
| VAR-B118 | 0.98 | 0.17 | 0.99 | 1.07 | 0.03 | 0.45 | 0.05 | 0.78 | 0.97 | 0.78 |
| VAR-B27 | 0.22 | 0.43 | 0.78 | 0.75 | 0.42 | 1.31 | 0.07 | 0.19 | 0.92 | 0.85 |
| VAR-B29 | 1.04 | 0.6 | 0.73 | 0.88 | 0.71 | 0.45 | 2.62 | 3.5 | 0.81 | 1.2 |
| VAR-B4 | 0.16 | 0.8 | 0.89 | 0.94 | 0.39 | 1.06 | 0.67 | 3.06 | 0.92 | 1.27 |
| VAR-B7 | 0.62 | 0.04 | 0.78 | 0.99 | 1.08 | 2.74 | 1.46 | 0.24 | 0.93 | 0.62 |
| VAR-B12 | 0.23 | 0.18 | 0.9 | 1.06 | 1.53 | 2.07 | 0.52 | 0.15 | 0.83 | 1.4 |
| VAR-B13 | 0.74 | 4.06 | 0.77 | 0.92 | 0.16 | 0 | 0 | 0 | 0.77 | 0.3 |
| VAR-B15 | 1.05 | 0.91 | 0.97 | 0.88 | 2.52 | 0.02 | 1.65 | 0 | 0.96 | 1.12 |
| VAR-B16 | 0.14 | 0 | 0.9 | 0.78 | 1.29 | 0 | 0.08 | 0.34 | 1.64 | 1.39 |
| VAR-B18 | 0.02 | 0.26 | 0.85 | 0.74 | 0.68 | 0 | 0.19 | 2.68 | 1.7 | 0.63 |
| VAR-B19 | 1.41 | 0 | 0.64 | 0.83 | 0.71 | 0.62 | 6.1 | 0 | 0.44 | 1.66 |
| VAR-B20 | 0 | 1.67 | 0.8 | 0.88 | 1.31 | 2.51 | 1.33 | 0.93 | 0.93 | 0.74 |
| VAR-B22 | 1.66 | 5.74 | 0.88 | 0.85 | 0.08 | 0.02 | 0.16 | 0.44 | 1.43 | 0.82 |
| VAR-B26 | 0.46 | 0.8 | 0.83 | 1.01 | 1.28 | 0.71 | 0.89 | 10.92 | 1.02 | 1.31 |
| VAR-B36 | 0.67 | 0 | 0.17 | 0.17 | 0.02 | 0 | 0.09 | 0 | 0.76 | 0.46 |
| VAR-B37 | 1.27 | 0.01 | 0.55 | 0.51 | 0.23 | 2.24 | 2.56 | 0 | 0.26 | 1.56 |
| VAR-B38 | 0.18 | 0 | 0.44 | 0.42 | 1.3 | 0.33 | 0.57 | 6.44 | 0.79 | 0.84 |
| VAR-B42 | 0.72 | 0.84 | 0.73 | 0.78 | 1.53 | 1.19 | 0.69 | 0.49 | 1.24 | 1.06 |
| VAR-B45 | 1.87 | 0.09 | 0.25 | 0.3 | 0.83 | 0.35 | 0.08 | 2.28 | 0.38 | 0.52 |
| VAR-B48 | 0.32 | 0.65 | 0.68 | 0.55 | 0.96 | 0.31 | 0.14 | 0.35 | 0.8 | 1.07 |
| VAR-B52 | 0.94 | 0 | 0.64 | 0.79 | 0.38 | 0.57 | 1.27 | 0 | 0.19 | 0.72 |
| VAR-B56 | 1.04 | 0.76 | 0.75 | 0.8 | 0.97 | 2.99 | 0.08 | 0.47 | 0.97 | 1.24 |
| VAR-B60 | 0.04 | 1.66 | 0.78 | 0.63 | 1.7 | 0 | 0 | 1.67 | 0.78 | 0.58 |
| VAR-B63 | 0.88 | 0.48 | 0.59 | 0.51 | 0.2 | 0.77 | 0.38 | 1.65 | 0.48 | 0.65 |
| VAR-B64 | 0 | 0 | 0.68 | 0.77 | 0 | 0 | 0.02 | 0 | 1.57 | 0.73 |
| VAR-B66 | 1 | 2.79 | 0.5 | 0.44 | 2.14 | 0.53 | 0.14 | 0.06 | 1.28 | 1.01 |
| VAR-B84 | 0.38 | 5.7 | 0.33 | 0.39 | 0.01 | 0 | 1.41 | 0 | 0.68 | 0.52 |
| VAR-B91 | 0.3 | 0.62 | 0.17 | 0.16 | 0 | 0 | 0.77 | 0 | 0.56 | 0.78 |
| VAR-B92 | 0.6 | 3.53 | 0.4 | 0.53 | 0.21 | 0 | 0 | 0 | 0.75 | 0.84 |
| VAR-B94 | 0.9 | 1.29 | 0.79 | 0.76 | 4.55 | 0 | 0.2 | 0 | 0.73 | 0.6 |
| VAR-B106 | 0.06 | 0.22 | 0.43 | 0.36 | 0.25 | 0 | 0.15 | 4.89 | 1.09 | 0.93 |
| VAR-B111 | 0 | 0 | 0.29 | 0.21 | 1.66 | 0.02 | 1.52 | 0 | 0.28 | 1.28 |
| VAR-B113 | 0.73 | 1.98 | 0.75 | 0.76 | 0.07 | 0 | 1.06 | 0.09 | 0.74 | 1.3 |
| VAR-B117 | 0.69 | 0.46 | 0.91 | 0.92 | 0.31 | 0.37 | 0.08 | 0 | 0.74 | 1.3 |
| VAR-B126 | 1.29 | 0 | 0.71 | 0.76 | 1.08 | 0.47 | 0.33 | 0 | 0.96 | 1.12 |

TABLE 19
Fold change in transduction of virus particles comprising the indicated variant capsid polypeptide, relative to the transduction
of virus particles comprising wild-type AAV9 in brain tissues. CBh indicates measurements from virus particles comprising
genomes with the cap gene under the control of the CBh promoter. hSyn indicates virus particles comprising genomes with the
cap gene under the control of the hSyn promoter. Values of “0” indicate variant transduction was not detected in the
indicated sample. Variants are ranked in order of fold improvement over WT AAV9 in aggregated brain from the hSyn promoter.

| | PROPERTY | | | | | | | |
|---|---|---|---|---|---|---|---|---|
| | Transduction | Transduction | Transduction | Transduction | Transduction | Transduction | Transduction | Transduction |
| | ORGAN | | | | | | | |
| | Brain | Brain | Brain | Brain | Brain | Brain | Brain | Brain |
| | TISSUE | | | | | | | |
| | | | Basal | Basal | Brain | Brain | | |
| | Aggregated | Aggregated | Ganglia | Ganglia | stem | stem | Cerebellum | Cerebellum |
| | PROMOTER | | | | | | | |
| VAR-# | CBh | hSyn | CBh | hSyn | CBh | hSyn | CBh | hSyn |
| VAR-B1 | 138.56 | 129.23 | 318.84 | 311.32 | 83.55 | 64.71 | 122.43 | 103.86 |
| VAR-B54 | 139.39 | 102.71 | 188.54 | 271.6 | 94.53 | 52.68 | 64.49 | 115.27 |
| VAR-B74 | 82.23 | 97.04 | 110.69 | 288.47 | 86.12 | 56.91 | 82.03 | 16.8 |
| VAR-B87 | 55.93 | 67.84 | 144.3 | 85.43 | 33.68 | 62.46 | 57.58 | 14.93 |
| VAR-B61 | 33.03 | 67.11 | 19.17 | 149.46 | 9.94 | 37.17 | 76.5 | 130.59 |
| VAR-B116 | 24.32 | 41.56 | 34.75 | 111.65 | 9.01 | 21.38 | 29.71 | 29.27 |
| VAR-B100 | 28.65 | 34.95 | 0 | 46.95 | 30.19 | 24.52 | 28.44 | 41.03 |
| VAR-B80 | 22.41 | 32.16 | 9.91 | 62.2 | 20.57 | 16.24 | 22.6 | 27.17 |
| VAR-B89 | 37.41 | 29.26 | 69.5 | 41.97 | 24.03 | 25.05 | 48.42 | 26.2 |
| VAR-B102 | 25.95 | 24.83 | 0 | 30.02 | 7.81 | 26.13 | 17.17 | 0 |
| VAR-B122 | 9.87 | 20.49 | 11.83 | 40.44 | 5.37 | 10.56 | 3.37 | 8.83 |
| VAR-B75 | 17.07 | 18.85 | 38.75 | 62.16 | 10.05 | 13.22 | 8.03 | 18.1 |
| VAR-B96 | 12.64 | 17.45 | 34.52 | 43.65 | 7.16 | 2.53 | 5.9 | 25.43 |
| VAR-B67 | 20.97 | 17.3 | 33.26 | 23.24 | 16.02 | 11.56 | 13.54 | 26.11 |
| VAR-B57 | 22.54 | 16.12 | 23.79 | 16.24 | 16.04 | 12.25 | 21.7 | 23.65 |
| VAR-B59 | 13.83 | 9.08 | 10.7 | 14.96 | 11.1 | 6.95 | 24.41 | 4.36 |
| VAR-B35 | 0 | 8.63 | 0 | 0 | 0 | 18.17 | 0 | 0 |
| VAR-B110 | 2.81 | 8.22 | 0 | 0 | 6.77 | 8.65 | 0 | 0 |
| VAR-B93 | 2.42 | 7.48 | 0 | 0 | 2.5 | 6.3 | 1.83 | 7.9 |
| VAR-B76 | 2.3 | 6.88 | 0 | 0 | 5.54 | 9.66 | 0 | 0 |
| VAR-B119 | 1.25 | 6.74 | 0 | 0 | 3 | 10.85 | 0 | 4.19 |
| VAR-B82 | 4.54 | 6.49 | 6.02 | 26.14 | 4.68 | 4.55 | 6.87 | 5.71 |
| VAR-B83 | 3.03 | 5.69 | 9.37 | 0 | 2.43 | 6.84 | 5.34 | 8.59 |
| VAR-B108 | 2.35 | 5.64 | 0 | 45.49 | 5.66 | 3.96 | 0 | 0 |
| VAR-B71 | 5.97 | 5.47 | 9.24 | 33.08 | 4.79 | 3.84 | 0 | 0 |
| VAR-B90 | 2.46 | 5.26 | 5.71 | 10.59 | 0 | 1.84 | 0 | 0 |
| VAR-B123 | 1.99 | 4.96 | 4.62 | 20.01 | 1.2 | 3.48 | 2.64 | 0 |
| VAR-B44 | 1.89 | 4.62 | 0 | 0 | 0 | 0 | 6.66 | 0 |
| VAR-B86 | 1.71 | 4.49 | 0 | 0 | 4.12 | 0 | 0 | 11.86 |
| VAR-B81 | 3.17 | 4 | 3.68 | 0 | 0.95 | 4.21 | 6.29 | 10.57 |
| VAR-B43 | 0 | 3.88 | 0 | 0 | 0 | 2.72 | 0 | 13.67 |
| VAR-B41 | 1.44 | 3.86 | 0 | 0 | 3.48 | 8.13 | 0 | 0 |
| VAR-B88 | 4.36 | 3.61 | 11.58 | 0 | 3 | 3.37 | 3.3 | 0 |
| VAR-B127 | 0 | 3.41 | 0 | 0 | 0 | 0 | 0 | 0 |
| VAR-B85 | 0.81 | 3.36 | 0 | 0 | 1.95 | 4.71 | 0 | 0 |
| VAR-B51 | 0 | 3.28 | 0 | 0 | 0 | 0 | 0 | 0 |
| VAR-B99 | 2.43 | 3.16 | 0 | 0 | 1.46 | 0 | 3.21 | 0 |
| VAR-B101 | 1.01 | 3.15 | 0 | 0 | 1.22 | 4.97 | 0 | 0 |
| VAR-B107 | 3.81 | 2.91 | 0 | 0 | 4.59 | 4.08 | 10.09 | 0 |
| VAR-B5 | 2.7 | 2.86 | 0 | 0 | 2.17 | 2 | 4.76 | 0 |
| VAR-B33 | 0.57 | 2.82 | 5.29 | 0 | 0 | 4.45 | 0 | 0 |
| VAR-B3 | 0 | 2.62 | 0 | 0 | 0 | 5.51 | 0 | 0 |
| VAR-B25 | 1.5 | 2.58 | 0 | 20.8 | 1.8 | 1.81 | 0 | 0 |
| VAR-B49 | 0 | 2.56 | 0 | 0 | 0 | 2.7 | 0 | 0 |
| VAR-B68 | 0.98 | 2.33 | 0 | 0 | 0 | 2.8 | 3.47 | 0 |
| VAR-B40 | 0 | 2.31 | 0 | 13.94 | 0 | 2.43 | 0 | 0 |
| VAR-B97 | 0 | 2.31 | 0 | 0 | 0 | 2.43 | 0 | 0 |
| VAR-B10 | 0.88 | 2.3 | 0 | 13.93 | 1.06 | 0 | 0 | 6.09 |
| VAR-B105 | 5.03 | 2.26 | 0 | 0 | 8.08 | 1.9 | 0 | 0 |
| VAR-B114 | 1.06 | 2.18 | 0 | 0 | 1.28 | 0 | 0 | 0 |
| VAR-B69 | 0 | 2.16 | 0 | 8.69 | 0 | 1.51 | 0 | 0 |
| VAR-B14 | 1.72 | 2.14 | 0 | 0 | 4.13 | 0 | 0 | 0 |
| VAR-B70 | 0.86 | 2.07 | 8.01 | 2 | 0 | 4.37 | 0 | 0 |
| VAR-B55 | 1.78 | 1.95 | 0 | 0 | 4.28 | 0 | 0 | 0 |
| VAR-B78 | 2.32 | 1.91 | 0 | 0 | 0 | 0 | 0 | 0 |
| VAR-B50 | 0.83 | 1.88 | 0 | 0 | 1 | 1.32 | 2.19 | 0 |
| VAR-B53 | 0 | 1.87 | 0 | 0 | 0 | 3.93 | 0 | 0 |
| VAR-B77 | 7.02 | 1.86 | 0 | 0 | 16.91 | 3.92 | 0 | 0 |
| VAR-B109 | 7.4 | 1.84 | 34.35 | 3 | 8.91 | 3.86 | 0 | 0 |
| VAR-B112 | 0.37 | 1.84 | 0 | 0 | 0.89 | 3.86 | 0 | 0 |
| VAR-B124 | 3.02 | 1.81 | 2.55 | 8.73 | 3.31 | 0.76 | 2.91 | 0 |
| VAR-B46 | 0 | 1.77 | 0 | 21.43 | 0 | 0 | 0 | 0 |
| VAR-B28 | 1 | 1.7 | 4.66 | 0 | 1.21 | 2.68 | 0 | 0 |
| VAR-B17 | 1.62 | 1.62 | 0 | 0 | 1.3 | 1.71 | 2.86 | 0 |
| VAR-B47 | 2.06 | 1.62 | 0 | 0 | 1.65 | 0 | 0 | 0 |
| VAR-B62 | 0.99 | 1.57 | 9.21 | 0 | 0 | 0 | 0 | 0 |
| VAR-B2 | 1.09 | 1.52 | 0 | 0 | 2.62 | 3.2 | 0 | 0 |
| VAR-B30 | 3.13 | 1.51 | 0 | 0 | 3.77 | 3.17 | 0 | 0 |
| VAR-B103 | 0 | 1.45 | 0 | 0 | 0 | 0 | 0 | 0 |
| VAR-B72 | 3.26 | 1.35 | 0 | 0 | 2.62 | 2.84 | 5.76 | 0 |
| VAR-B23 | 1.16 | 1.34 | 0 | 0 | 1.4 | 0 | 0 | 7.1 |
| VAR-B95 | 6.92 | 1.34 | 12.85 | 0 | 3.33 | 0 | 7.33 | 14.11 |
| VAR-B73 | 0 | 1.27 | 0 | 0 | 0 | 2.66 | 0 | 0 |
| VAR-B79 | 1.8 | 1.21 | 8.34 | 14.63 | 2.16 | 0 | 0 | 0 |
| VAR-B115 | 2.63 | 1.16 | 6.1 | 0 | 0 | 1.22 | 3.48 | 0 |
| VAR-B98 | 0.98 | 1.15 | 0 | 0 | 2.36 | 2.41 | 0 | 0 |
| VAR-B125 | 1.39 | 1.05 | 0 | 0 | 3.35 | 0 | 0 | 0 |
| VAR-B104 | 0 | 1.03 | 0 | 0 | 0 | 2.16 | 0 | 0 |
| VAR-B31 | 1.34 | 0.99 | 0 | 0 | 1.08 | 2.08 | 2.36 | 0 |
| VAR-B24 | 1.43 | 0.97 | 0 | 0 | 0 | 0 | 0 | 0 |
| VAR-B39 | 0 | 0.96 | 0 | 0 | 0 | 0 | 0 | 0 |
| VAR-B8 | 1.5 | 0.94 | 0 | 0 | 0 | 1.97 | 0 | 0 |
| VAR-B11 | 2.94 | 0.91 | 0 | 0 | 0 | 0 | 0 | 0 |
| VAR-B32 | 0.91 | 0.8 | 4.21 | 0 | 0 | 1.68 | 0 | 0 |
| VAR-B6 | 0.37 | 0.76 | 0 | 0 | 0 | 1.59 | 1.98 | 0 |
| VAR-B121 | 0.51 | 0.76 | 0 | 0 | 0.62 | 0.53 | 1.36 | 2.66 |
| VAR-B9 | 1.02 | 0.73 | 4.75 | 0 | 1.23 | 1.54 | 0 | 0 |
| VAR-B65 | 1.29 | 0.65 | 0 | 0 | 3.11 | 0 | 0 | 0 |
| VAR-B21 | 1.03 | 0.62 | 0 | 0 | 1.24 | 1.3 | 2.73 | 0 |
| VAR-B58 | 1 | 0.59 | 3.1 | 0 | 0.8 | 0 | 0 | 0 |
| VAR-B34 | 1.04 | 0.58 | 0 | 0 | 0 | 1.22 | 0 | 0 |
| VAR-B120 | 0.57 | 0.54 | 0 | 0 | 0.68 | 0 | 0 | 0 |
| VAR-B118 | 0 | 0.53 | 0 | 0 | 0 | 0 | 0 | 0 |
| VAR-B27 | 0 | 0.51 | 0 | 0 | 0 | 0 | 0 | 0 |
| VAR-B29 | 1.45 | 0.46 | 8.98 | 0 | 0 | 0 | 2.56 | 0 |
| VAR-B4 | 0 | 0 | 0 | 0 | 0 | 0 | 0 | 0 |
| VAR-B7 | 2.87 | 0 | 0 | 0 | 3.45 | 0 | 3.79 | 0 |
| VAR-B12 | 1.89 | 0 | 0 | 0 | 2.28 | 0 | 0 | 0 |
| VAR-B13 | 4.84 | 0 | 0 | 0 | 9.33 | 0 | 5.13 | 0 |
| VAR-B15 | 0 | 0 | 0 | 0 | 0 | 0 | 0 | 0 |
| VAR-B16 | 0 | 0 | 0 | 0 | 0 | 0 | 0 | 0 |
| VAR-B18 | 11.04 | 0 | 0 | 0 | 26.6 | 0 | 0 | 0 |
| VAR-B19 | 0 | 0 | 0 | 0 | 0 | 0 | 0 | 0 |
| VAR-B20 | 1.64 | 0 | 0 | 0 | 0 | 0 | 8.71 | 0 |
| VAR-B22 | 0 | 0 | 0 | 0 | 0 | 0 | 0 | 0 |
| VAR-B26 | 2.33 | 0 | 7.2 | 0 | 0 | 0 | 8.21 | 0 |
| VAR-B36 | 1.42 | 0 | 0 | 0 | 3.42 | 0 | 0 | 0 |
| VAR-B37 | 0 | 0 | 0 | 0 | 0 | 0 | 0 | 0 |
| VAR-B38 | 1.4 | 0 | 13.04 | 0 | 0 | 0 | 0 | 0 |
| VAR-B42 | 2.93 | 0 | 5.45 | 0 | 4.24 | 0 | 3.11 | 0 |
| VAR-B45 | 0 | 0 | 0 | 0 | 0 | 0 | 0 | 0 |
| VAR-B48 | 0.57 | 0 | 0 | 0 | 0 | 0 | 0 | 0 |
| VAR-B52 | 0 | 0 | 0 | 0 | 0 | 0 | 0 | 0 |
| VAR-B56 | 1.19 | 0 | 0 | 0 | 1.43 | 0 | 3.14 | 0 |
| VAR-B60 | 0 | 0 | 0 | 0 | 0 | 0 | 0 | 0 |
| VAR-B63 | 0 | 0 | 0 | 0 | 0 | 0 | 0 | 0 |
| VAR-B64 | 6.27 | 0 | 0 | 0 | 0 | 0 | 0 | 0 |
| VAR-B66 | 4.98 | 0 | 0 | 0 | 4.8 | 0 | 5.27 | 0 |
| VAR-B84 | 0 | 0 | 0 | 0 | 0 | 0 | 0 | 0 |
| VAR-B91 | 0 | 0 | 0 | 0 | 0 | 0 | 0 | 0 |
| VAR-B92 | 0 | 0 | 0 | 0 | 0 | 0 | 0 | 0 |
| VAR-B94 | 0 | 0 | 0 | 0 | 0 | 0 | 0 | 0 |
| VAR-B106 | 4.24 | 0 | 19.71 | 0 | 5.11 | 0 | 0 | 0 |
| VAR-B111 | 2.26 | 0 | 0 | 0 | 5.44 | 0 | 0 | 0 |
| VAR-B113 | 2.13 | 0 | 0 | 0 | 5.13 | 0 | 0 | 0 |
| VAR-B117 | 0 | 0 | 0 | 0 | 0 | 0 | 0 | 0 |
| VAR-B126 | 0 | 0 | 0 | 0 | 0 | 0 | 0 | 0 |

| | PROPERTY | | | | | | | |
|---|---|---|---|---|---|---|---|---|
| | Transduction | Transduction | Transduction | Transduction | Transduction | Transduction | Transduction | Transduction |
| | ORGAN | | | | | | | |
| | Brain | Brain | Brain | Brain | Brain | Brain | Brain | Brain |
| | TISSUE | | | | | | | |
| | | | | | | | Temporal | Temporal |
| | Forebrain | Forebrain | Hippocampus | Hippocampus | Mid-brain | Mid-brain | Cortex | Cortex |
| | PROMOTER | | | | | | | |
| VAR-# | CBh | hSyn | CBh | hSyn | CBh | hSyn | CBh | hSyn |
| VAR-B1 | 183.47 | 179.59 | 0 | 339.62 | 159.13 | 186.79 | 129.25 | 165.4 |
| VAR-B54 | 244.29 | 106.34 | 0 | 0 | 121.07 | 335.24 | 290.19 | 72.56 |
| VAR-B74 | 92.2 | 135.33 | 0 | 0 | 79.97 | 173.08 | 14.2 | 89.91 |
| VAR-B87 | 50.08 | 56.95 | 340.56 | 0 | 34.75 | 215.28 | 55.53 | 99.85 |
| VAR-B61 | 66.54 | 71.17 | 0 | 0 | 46.17 | 76.86 | 0 | 74.87 |
| VAR-B116 | 36.18 | 70.3 | 0 | 0 | 125.51 | 40.19 | 0 | 26.1 |
| VAR-B100 | 57.71 | 60.86 | 0 | 0 | 0 | 42.26 | 21.33 | 0 |
| VAR-B80 | 41.29 | 48.95 | 0 | 0 | 47.75 | 69.98 | 0 | 36.35 |
| VAR-B89 | 42.88 | 31.09 | 0 | 0 | 37.19 | 32.38 | 29.71 | 42.05 |
| VAR-B102 | 104.55 | 33.35 | 0 | 0 | 72.55 | 0 | 0 | 35.09 |
| VAR-B122 | 24.64 | 26.21 | 0 | 0 | 7.12 | 18.2 | 18.97 | 65 |
| VAR-B75 | 26.9 | 25.58 | 0 | 0 | 25.45 | 0 | 22.59 | 0 |
| VAR-B96 | 28.75 | 29.64 | 0 | 0 | 8.31 | 26.19 | 0 | 34.01 |
| VAR-B67 | 42.87 | 17.21 | 0 | 0 | 34.32 | 17.93 | 0 | 38.8 |
| VAR-B57 | 46.24 | 20.05 | 0 | 0 | 34.38 | 48.72 | 6.1 | 0 |
| VAR-B59 | 14.86 | 11.09 | 0 | 0 | 0 | 17.96 | 13.73 | 11.66 |
| VAR-B35 | 0 | 0 | 0 | 0 | 0 | 0 | 0 | 0 |
| VAR-B110 | 0 | 0 | 0 | 0 | 0 | 89.39 | 0 | 0 |
| VAR-B93 | 2.23 | 0 | 0 | 0 | 7.73 | 65.13 | 4.12 | 10.57 |
| VAR-B76 | 0 | 10.27 | 0 | 0 | 0 | 0 | 0 | 0 |
| VAR-B119 | 0 | 5.33 | 0 | 0 | 0 | 0 | 0 | 0 |
| VAR-B82 | 2.09 | 4.84 | 0 | 0 | 0 | 11.76 | 3.86 | 0 |
| VAR-B83 | 0 | 3.64 | 0 | 0 | 0 | 0 | 0 | 11.49 |
| VAR-B108 | 0 | 0 | 0 | 0 | 0 | 0 | 0 | 0 |
| VAR-B71 | 6.41 | 0 | 0 | 0 | 0 | 19.85 | 23.69 | 0 |
| VAR-B90 | 3.96 | 11.77 | 0 | 119.18 | 13.75 | 0 | 7.32 | 0 |
| VAR-B123 | 3.21 | 7.41 | 0 | 0 | 0 | 0 | 0 | 0 |
| VAR-B44 | 4.06 | 20.67 | 0 | 0 | 0 | 0 | 0 | 0 |
| VAR-B86 | 0 | 10.06 | 0 | 0 | 0 | 0 | 0 | 15.87 |
| VAR-B81 | 5.1 | 4.48 | 0 | 0 | 0 | 0 | 4.71 | 0 |
| VAR-B43 | 0 | 5.79 | 0 | 0 | 0 | 0 | 0 | 0 |
| VAR-B41 | 0 | 0 | 0 | 0 | 0 | 0 | 0 | 0 |
| VAR-B88 | 8.04 | 7.17 | 0 | 0 | 0 | 8.72 | 0 | 0 |
| VAR-B127 | 0 | 0 | 0 | 0 | 0 | 0 | 0 | 48.18 |
| VAR-B85 | 0 | 0 | 0 | 0 | 0 | 0 | 0 | 15.81 |
| VAR-B51 | 0 | 14.7 | 0 | 0 | 0 | 0 | 0 | 0 |
| VAR-B99 | 3.91 | 0 | 0 | 0 | 13.57 | 68.69 | 0 | 0 |
| VAR-B101 | 3.27 | 3.52 | 0 | 0 | 0 | 0 | 0 | 0 |
| VAR-B107 | 0 | 4.34 | 0 | 0 | 0 | 0 | 0 | 0 |
| VAR-B5 | 0 | 4.26 | 98.59 | 0 | 0 | 0 | 5.36 | 13.45 |
| VAR-B33 | 0 | 3.15 | 0 | 0 | 0 | 0 | 0 | 0 |
| VAR-B3 | 0 | 0 | 0 | 0 | 0 | 0 | 0 | 0 |
| VAR-B25 | 0 | 0 | 0 | 0 | 0 | 0 | 8.92 | 0 |
| VAR-B49 | 0 | 0 | 0 | 0 | 0 | 0 | 0 | 18.1 |
| VAR-B68 | 2.11 | 1.49 | 0 | 0 | 0 | 0 | 0 | 9.4 |
| VAR-B40 | 0 | 0 | 0 | 0 | 0 | 0 | 0 | 0 |
| VAR-B97 | 0 | 5.17 | 0 | 0 | 0 | 0 | 0 | 0 |
| VAR-B10 | 0 | 0 | 0 | 0 | 0 | 0 | 5.23 | 8.14 |
| VAR-B105 | 5.41 | 6.07 | 0 | 0 | 18.76 | 0 | 0 | 0 |
| VAR-B114 | 3.42 | 6.5 | 0 | 0 | 0 | 15.8 | 0 | 0 |
| VAR-B69 | 0 | 3.22 | 0 | 0 | 0 | 0 | 0 | 0 |
| VAR-B14 | 0 | 9.57 | 0 | 0 | 0 | 0 | 0 | 0 |
| VAR-B70 | 0 | 0 | 0 | 0 | 0 | 0 | 0 | 0 |
| VAR-B55 | 0 | 8.72 | 0 | 0 | 0 | 0 | 0 | 0 |
| VAR-B78 | 0 | 0 | 0 | 0 | 0 | 41.66 | 27.59 | 0 |
| VAR-B50 | 0 | 5.62 | 0 | 0 | 0 | 0 | 0 | 0 |
| VAR-B53 | 0 | 0 | 0 | 0 | 0 | 0 | 0 | 0 |
| VAR-B77 | 0 | 0 | 0 | 0 | 0 | 0 | 0 | 0 |
| VAR-B109 | 0 | 0 | 0 | 0 | 0 | 0 | 0 | 0 |
| VAR-B112 | 0 | 0 | 0 | 0 | 0 | 0 | 0 | 0 |
| VAR-B124 | 3.54 | 1.62 | 60.19 | 0 | 0 | 7.86 | 0 | 0 |
| VAR-B46 | 0 | 0 | 0 | 0 | 0 | 0 | 0 | 0 |
| VAR-B28 | 0 | 1.9 | 0 | 0 | 0 | 0 | 0 | 0 |
| VAR-B17 | 3.49 | 3.64 | 0 | 0 | 0 | 0 | 0 | 0 |
| VAR-B47 | 8.86 | 2.42 | 0 | 0 | 0 | 11.78 | 0 | 7.65 |
| VAR-B62 | 0 | 7.02 | 0 | 0 | 0 | 0 | 0 | 0 |
| VAR-B2 | 0 | 0 | 0 | 0 | 0 | 0 | 0 | 0 |
| VAR-B30 | 10.1 | 0 | 0 | 0 | 0 | 0 | 0 | 0 |
| VAR-B103 | 0 | 6.48 | 0 | 0 | 0 | 0 | 0 | 0 |
| VAR-B72 | 0 | 0 | 0 | 0 | 0 | 0 | 12.95 | 0 |
| VAR-B23 | 3.75 | 0 | 0 | 0 | 0 | 14.63 | 0 | 0 |
| VAR-B95 | 8.92 | 0 | 0 | 0 | 15.47 | 0 | 8.24 | 0 |
| VAR-B73 | 0 | 0 | 0 | 0 | 0 | 0 | 0 | 0 |
| VAR-B79 | 0 | 0 | 0 | 0 | 0 | 0 | 0 | 0 |
| VAR-B115 | 0 | 2.6 | 0 | 0 | 29.37 | 0 | 0 | 0 |
| VAR-B98 | 0 | 0 | 0 | 0 | 0 | 0 | 0 | 0 |
| VAR-B125 | 0 | 4.68 | 0 | 0 | 0 | 0 | 0 | 0 |
| VAR-B104 | 0 | 0 | 0 | 0 | 0 | 0 | 0 | 0 |
| VAR-B31 | 0 | 0 | 97.9 | 0 | 0 | 0 | 0 | 0 |
| VAR-B24 | 9.2 | 4.33 | 0 | 0 | 0 | 0 | 0 | 0 |
| VAR-B39 | 0 | 0 | 0 | 0 | 0 | 0 | 0 | 13.53 |
| VAR-B8 | 0 | 0 | 0 | 0 | 33.59 | 0 | 0 | 0 |
| VAR-B11 | 18.96 | 4.08 | 0 | 0 | 0 | 0 | 0 | 0 |
| VAR-B32 | 0 | 0 | 0 | 0 | 0 | 0 | 5.4 | 0 |
| VAR-B6 | 0 | 0 | 0 | 0 | 0 | 0 | 0 | 0 |
| VAR-B121 | 0 | 1.13 | 0 | 0 | 0 | 0 | 0 | 0 |
| VAR-B9 | 0 | 0 | 0 | 0 | 0 | 0 | 0 | 0 |
| VAR-B65 | 0 | 2.91 | 0 | 0 | 0 | 0 | 0 | 0 |
| VAR-B21 | 0 | 0 | 0 | 0 | 0 | 0 | 0 | 0 |
| VAR-B58 | 2.15 | 2.65 | 0 | 0 | 0 | 0 | 0 | 0 |
| VAR-B34 | 6.69 | 0 | 0 | 0 | 0 | 0 | 0 | 0 |
| VAR-B120 | 1.83 | 0 | 0 | 0 | 0 | 0 | 0 | 7.63 |
| VAR-B118 | 0 | 2.36 | 0 | 0 | 0 | 0 | 0 | 0 |
| VAR-B27 | 0 | 2.27 | 0 | 0 | 0 | 0 | 0 | 0 |
| VAR-B29 | 0 | 2.07 | 0 | 0 | 0 | 0 | 0 | 0 |
| VAR-B4 | 0 | 0 | 0 | 0 | 0 | 0 | 0 | 0 |
| VAR-B7 | 0 | 0 | 0 | 0 | 16.03 | 0 | 0 | 0 |
| VAR-B12 | 6.1 | 0 | 0 | 0 | 0 | 0 | 0 | 0 |
| VAR-B13 | 0 | 0 | 0 | 0 | 0 | 0 | 0 | 0 |
| VAR-B15 | 0 | 0 | 0 | 0 | 0 | 0 | 0 | 0 |
| VAR-B16 | 0 | 0 | 0 | 0 | 0 | 0 | 0 | 0 |
| VAR-B18 | 0 | 0 | 0 | 0 | 0 | 0 | 0 | 0 |
| VAR-B19 | 0 | 0 | 0 | 0 | 0 | 0 | 0 | 0 |
| VAR-B20 | 0 | 0 | 0 | 0 | 0 | 0 | 0 | 0 |
| VAR-B22 | 0 | 0 | 0 | 0 | 0 | 0 | 0 | 0 |
| VAR-B26 | 0 | 0 | 0 | 0 | 0 | 0 | 0 | 0 |
| VAR-B36 | 0 | 0 | 0 | 0 | 0 | 0 | 0 | 0 |
| VAR-B37 | 0 | 0 | 0 | 0 | 0 | 0 | 0 | 0 |
| VAR-B38 | 0 | 0 | 0 | 0 | 0 | 0 | 0 | 0 |
| VAR-B42 | 0 | 0 | 0 | 0 | 0 | 0 | 0 | 0 |
| VAR-B45 | 0 | 0 | 0 | 0 | 0 | 0 | 0 | 0 |
| VAR-B48 | 3.67 | 0 | 0 | 0 | 0 | 0 | 0 | 0 |
| VAR-B52 | 0 | 0 | 0 | 0 | 0 | 0 | 0 | 0 |
| VAR-B56 | 0 | 0 | 0 | 0 | 0 | 0 | 0 | 0 |
| VAR-B60 | 0 | 0 | 0 | 0 | 0 | 0 | 0 | 0 |
| VAR-B63 | 0 | 0 | 0 | 0 | 0 | 0 | 0 | 0 |
| VAR-B64 | 40.4 | 0 | 0 | 0 | 0 | 0 | 0 | 0 |
| VAR-B66 | 6.42 | 0 | 0 | 0 | 22.28 | 0 | 0 | 0 |
| VAR-B84 | 0 | 0 | 0 | 0 | 0 | 0 | 0 | 0 |
| VAR-B91 | 0 | 0 | 0 | 0 | 0 | 0 | 0 | 0 |
| VAR-B92 | 0 | 0 | 0 | 0 | 0 | 0 | 0 | 0 |
| VAR-B94 | 0 | 0 | 0 | 0 | 0 | 0 | 0 | 0 |
| VAR-B106 | 0 | 0 | 0 | 0 | 0 | 0 | 0 | 0 |
| VAR-B111 | 0 | 0 | 0 | 0 | 0 | 0 | 0 | 0 |
| VAR-B113 | 0 | 0 | 0 | 0 | 0 | 0 | 0 | 0 |
| VAR-B117 | 0 | 0 | 0 | 0 | 0 | 0 | 0 | 0 |
| VAR-B126 | 0 | 0 | 0 | 0 | 0 | 0 | 0 | 0 |

TABLE 20
Fold change in transduction of virus particles comprising the indicated variant capsid polypeptide, relative to the transduction of virus particles comprising
wild-type AAV9 in non-brain tissues. CBh indicates measurements from virus particles comprising genomes with the cap gene under the control of the CBh promoter.
hSyn indicates virus particles comprising genomes with the cap gene under the control of the hSyn promoter. Values of “0” indicate variant transduction
was not detected in the indicated sample. Variants are ranked in order of fold improvement over WT AAV9 in aggregated brain from the hSyn promoter.

PROPERTY

| | | | | | | | | | |
|---|---|---|---|---|---|---|---|---|---|
| Trans- | Trans- | Trans- | Trans- | Trans- | Trans- | Trans- | Trans- | Trans- | Trans- |
| duction | duction | duction | duction | duction | duction | duction | duction | duction | duction |

ORGAN

| | | | | | | | | | |
|---|---|---|---|---|---|---|---|---|---|
| Dorsal Root | Dorsal Root | | | Skeletal | Skeletal | | | | |
| Ganglion | Ganglion | Liver | Liver | Muscle | Muscle | Spinal Cord | Spinal Cord | Spleen | Spleen |

TISSUE

| | | | | | | | | | |
|---|---|---|---|---|---|---|---|---|---|
| Aggregated | Aggregated | Aggregated | Aggregated | Aggregated | Aggregated | Aggregated | Aggregated | Aggregated | Aggregated |

PROMOTER

| VAR# | CBh | hSyn | CBh | hSyn | CBh | hSyn | CBh | hSyn | CBh | hSyn |
|---|---|---|---|---|---|---|---|---|---|---|
| VAR-B1 | 1.76 | 1.78 | 0.32 | 0.52 | 1.19 | 1.47 | 46.06 | 63.23 | 0 | 2.36 |
| VAR-B54 | 0 | 2.43 | 0.3 | 0.4 | 2.65 | 1.73 | 89.64 | 48.29 | 0 | 0 |
| VAR-B74 | 5.88 | 0 | 0.17 | 0.5 | 1.33 | 0 | 50.24 | 18.41 | 0 | 0 |
| VAR-B87 | 0 | 0 | 1.02 | 1.03 | 1.3 | 0 | 9.36 | 8.18 | 0 | 0 |
| VAR-B61 | 0 | 0 | 0.45 | 0.18 | 2.89 | 0 | 12.43 | 10.22 | 0 | 0 |
| VAR-B116 | 0 | 3.5 | 1.09 | 0.86 | 2.61 | 0 | 11.26 | 32.07 | 0 | 0 |
| VAR-B100 | 0 | 14.72 | 1.04 | 0.81 | 1.5 | 0 | 21.56 | 33.71 | 0 | 0 |
| VAR-B80 | 0 | 0 | 0.23 | 0.47 | 1.79 | 3.47 | 6.43 | 11.17 | 0 | 0 |
| VAR-B89 | 0 | 0 | 0.61 | 0.52 | 1.16 | 0.67 | 10.01 | 17.22 | 0 | 0 |
| VAR-B102 | 16.01 | 0 | 0.24 | 0.26 | 0 | 0 | 19.53 | 28.74 | 0 | 0 |
| VAR-B122 | 3.14 | 3.17 | 0.28 | 0.3 | 0.8 | 0.56 | 7.67 | 2.42 | 0 | 0 |
| VAR-B75 | 0 | 0 | 0.36 | 0.71 | 1.8 | 0 | 2.28 | 6.61 | 0 | 0 |
| VAR-B96 | 3.67 | 4.56 | 0.16 | 0.19 | 1.45 | 0 | 6.71 | 3.48 | 0 | 0 |
| VAR-B67 | 0 | 0 | 1.04 | 1.11 | 1.57 | 0.37 | 12.32 | 9.54 | 0 | 0 |
| VAR-B57 | 0 | 0 | 0.45 | 1.07 | 1.72 | 1.21 | 15.43 | 2.59 | 4.8 | 6.77 |
| VAR-B59 | 0 | 1.56 | 0.84 | 0.6 | 0.97 | 0 | 6.94 | 7.16 | 10.8 | 6.24 |
| VAR-B35 | 0 | 0 | 0 | 1.79 | 0 | 0 | 0 | 0 | 0 | 0 |
| VAR-B110 | 0 | 0 | 0.41 | 0.85 | 0 | 5.54 | 0 | 0 | 0 | 0 |
| VAR-B93 | 5.12 | 0 | 0.68 | 1.4 | 1.64 | 1.01 | 4.16 | 0 | 0 | 0 |
| VAR-B76 | 0 | 0 | 1.51 | 1.43 | 1.93 | 3.09 | 0 | 0 | 0 | 0 |
| VAR-B119 | 4.1 | 0 | 0.61 | 0.7 | 0.81 | 3.74 | 2.5 | 4.59 | 0 | 0 |
| VAR-B82 | 0 | 2.05 | 1.04 | 1.52 | 1.09 | 0 | 3.9 | 3.13 | 0 | 0 |
| VAR-B83 | 0 | 0 | 1.1 | 1.27 | 0.56 | 2.19 | 0 | 4.7 | 0 | 0 |
| VAR-B108 | 0 | 0 | 0.34 | 1.17 | 2.63 | 0 | 0 | 10.89 | 0 | 0 |
| VAR-B71 | 0 | 3.46 | 0.94 | 2.27 | 0.56 | 1.23 | 5.99 | 0 | 0 | 0 |
| VAR-B90 | 0 | 0 | 0.72 | 0.55 | 1.03 | 1.18 | 0 | 0 | 0 | 0 |
| VAR-B123 | 2.46 | 0 | 0.8 | 0.86 | 0.97 | 4.46 | 9 | 9.58 | 0 | 25.01 |
| VAR-B44 | 0 | 0 | 1.29 | 0.64 | 0.35 | 2.07 | 0 | 0 | 0 | 0 |
| VAR-B86 | 0 | 4.26 | 0.83 | 1.75 | 1.28 | 1.51 | 0 | 0 | 10.69 | 0 |
| VAR-B81 | 1.95 | 0 | 0.81 | 0.31 | 1.22 | 0 | 2.38 | 0 | 0 | 0 |
| VAR-B43 | 0 | 0 | 0.4 | 1.88 | 0.77 | 1.74 | 0 | 0 | 0 | 0 |
| VAR-B41 | 0 | 0 | 0.46 | 0.48 | 0.54 | 0 | 2.9 | 0 | 0 | 0 |
| VAR-B88 | 3.08 | 0 | 0.5 | 0.29 | 0.52 | 1.08 | 3.75 | 2.32 | 0 | 0 |
| VAR-B127 | 0 | 0 | 1.04 | 1.06 | 0 | 0 | 0 | 0 | 0 | 0 |
| VAR-B85 | 2 | 0 | 0.86 | 0.81 | 1.36 | 4.52 | 0 | 0 | 0 | 0 |
| VAR-B51 | 0 | 12.44 | 1.06 | 1.02 | 0.51 | 0 | 10.91 | 19 | 0 | 0 |
| VAR-B99 | 3 | 11.97 | 0.58 | 1.31 | 2.04 | 0 | 7.31 | 0 | 0 | 0 |
| VAR-B101 | 0 | 0 | 0.55 | 0.74 | 0.71 | 2.12 | 3.05 | 0 | 0 | 0 |
| VAR-B107 | 0 | 0 | 0.19 | 0.71 | 0.53 | 0 | 0 | 11.22 | 0 | 0 |
| VAR-B5 | 0 | 10.83 | 0.56 | 0.3 | 0.75 | 0 | 2.71 | 0 | 0 | 0 |
| VAR-B33 | 8.44 | 5.34 | 0.71 | 0.37 | 1.12 | 1.9 | 0 | 8.15 | 0 | 0 |
| VAR-B3 | 0 | 0 | 1.4 | 1.22 | 0.54 | 0 | 0 | 7.57 | 0 | 0 |
| VAR-B25 | 0 | 0 | 0.38 | 0.54 | 1.05 | 0 | 0 | 4.98 | 0 | 0 |
| VAR-B49 | 0 | 0 | 0.5 | 0.8 | 0 | 0 | 0 | 0 | 0 | 0 |
| VAR-B68 | 1.62 | 2.52 | 0.86 | 1.21 | 1.1 | 0.9 | 0 | 1.92 | 3.07 | 0 |
| VAR-B40 | 8.14 | 0 | 0.12 | 0.6 | 0.46 | 0 | 0 | 0 | 0 | 0 |
| VAR-B97 | 0 | 0 | 1.04 | 1.2 | 0 | 0 | 0 | 0 | 0 | 0 |
| VAR-B10 | 0 | 0 | 0.77 | 0.54 | 0.86 | 0.78 | 0 | 0 | 0 | 0 |
| VAR-B105 | 0 | 1.71 | 0.24 | 0.52 | 0.94 | 3.05 | 0 | 0 | 0 | 0 |
| VAR-B114 | 2.62 | 0 | 0.77 | 0.75 | 0.74 | 1.96 | 3.19 | 4.2 | 0 | 0 |
| VAR-B69 | 4.62 | 2.73 | 1.23 | 1.57 | 0.79 | 0.97 | 0 | 0 | 0 | 0 |
| VAR-B14 | 0 | 0 | 1.38 | 0.44 | 1.44 | 12.88 | 0 | 0 | 0 | 0 |
| VAR-B70 | 0 | 1.97 | 0.76 | 1.08 | 1.45 | 2.1 | 0 | 0 | 0 | 7.84 |
| VAR-B55 | 0 | 0 | 0.84 | 0.4 | 0.5 | 0 | 0 | 0 | 0 | 0 |
| VAR-B78 | 0 | 0 | 1.35 | 1.59 | 1.94 | 2.58 | 0 | 0 | 0 | 0 |
| VAR-B50 | 4.09 | 0 | 0.33 | 0.72 | 1.04 | 0.85 | 0 | 0 | 0 | 0 |
| VAR-B53 | 0 | 0 | 0.39 | 0.58 | 1.5 | 0 | 0 | 0 | 0 | 0 |
| VAR-B77 | 0 | 0 | 0.34 | 0.19 | 0 | 0 | 0 | 0 | 0 | 0 |
| VAR-B109 | 0 | 0 | 0.4 | 1.05 | 0.52 | 0 | 0 | 0 | 0 | 0 |
| VAR-B112 | 0 | 0 | 1.08 | 0.95 | 1.04 | 1.24 | 0 | 0 | 0 | 0 |
| VAR-B124 | 2.71 | 2.74 | 1.42 | 1.54 | 1.23 | 0.97 | 0 | 0 | 2.57 | 0 |
| VAR-B46 | 0 | 0 | 0.39 | 0.37 | 0.99 | 0 | 0 | 0 | 0 | 0 |
| VAR-B28 | 0 | 0 | 0.29 | 0.62 | 0 | 0.57 | 0 | 4.91 | 0 | 0 |
| VAR-B17 | 0 | 0 | 0.47 | 1.18 | 1.81 | 2.19 | 0 | 0 | 0 | 0 |
| VAR-B47 | 0 | 0 | 0.8 | 1.07 | 1.54 | 1.46 | 0 | 0 | 0 | 0 |
| VAR-B62 | 0 | 2.97 | 0.43 | 0.57 | 0.28 | 0 | 0 | 0 | 0 | 0 |
| VAR-B2 | 0 | 5.77 | 1.03 | 0.63 | 1.22 | 0 | 0 | 0 | 0 | 0 |
| VAR-B30 | 7.74 | 5.71 | 0.69 | 0.78 | 0 | 0 | 0 | 0 | 0 | 0 |
| VAR-B103 | 0 | 0 | 0.93 | 1.05 | 0 | 1.95 | 0 | 0 | 0 | 0 |
| VAR-B72 | 0 | 0 | 0.4 | 0.28 | 0.3 | 0 | 0 | 7.8 | 0 | 0 |
| VAR-B23 | 0 | 2.55 | 0.13 | 0.35 | 1.46 | 0 | 3.5 | 3.89 | 0 | 0 |
| VAR-B95 | 0 | 0 | 0.3 | 0.28 | 2.13 | 3.6 | 0 | 0 | 0 | 0 |
| VAR-B73 | 0 | 4.8 | 1.91 | 0.53 | 0 | 1.71 | 0 | 0 | 0 | 0 |
| VAR-B79 | 4.43 | 4.59 | 0.26 | 0.5 | 0.5 | 0 | 0 | 0 | 0 | 0 |
| VAR-B115 | 0 | 0 | 0.53 | 0.84 | 1.29 | 2.35 | 0 | 0 | 0 | 0 |
| VAR-B98 | 0 | 2.17 | 0.79 | 1.13 | 0.55 | 0.77 | 5.91 | 0 | 0 | 0 |
| VAR-B125 | 0 | 0 | 0.81 | 0.76 | 2.34 | 1.41 | 0 | 0 | 13.04 | 15.81 |
| VAR-B104 | 0 | 3.9 | 1.07 | 0.96 | 0.27 | 1.39 | 0 | 0 | 0 | 0 |
| VAR-B31 | 4.41 | 1.87 | 0.85 | 0.51 | 1 | 3.32 | 2.69 | 2.85 | 0 | 0 |
| VAR-B24 | 0 | 0 | 0.68 | 1 | 0 | 0 | 0 | 0 | 0 | 0 |
| VAR-B39 | 0 | 0 | 0 | 0.5 | 0.44 | 0 | 0 | 0 | 0 | 0 |
| VAR-B8 | 0 | 0 | 0.66 | 0.78 | 1.26 | 0 | 0 | 5.43 | 0 | 0 |
| VAR-B11 | 0 | 0 | 0.64 | 0.66 | 0.82 | 0 | 0 | 0 | 0 | 0 |
| VAR-B32 | 0 | 0 | 0.43 | 1.08 | 1.27 | 0 | 0 | 0 | 0 | 0 |
| VAR-B6 | 1.85 | 0 | 0.79 | 0.91 | 1.26 | 0 | 2.25 | 0 | 0 | 5.73 |
| VAR-B121 | 1.27 | 0 | 0.86 | 0.63 | 1.22 | 1.36 | 1.55 | 0 | 0 | 3.81 |
| VAR-B9 | 2.53 | 0 | 1.19 | 0.76 | 1.29 | 0 | 3.08 | 0 | 0 | 11.07 |
| VAR-B65 | 0 | 2.46 | 0.66 | 0.74 | 1.81 | 0 | 0 | 0 | 0 | 0 |
| VAR-B21 | 0 | 4.68 | 0.34 | 1.03 | 1.73 | 0 | 0 | 3.57 | 0 | 9.33 |
| VAR-B58 | 1.65 | 4.48 | 1.1 | 0.98 | 1.03 | 1.59 | 0 | 0 | 0 | 0 |
| VAR-B34 | 0 | 0 | 0.19 | 0.48 | 0.43 | 0.78 | 3.12 | 0 | 0 | 0 |
| VAR-B120 | 1.4 | 0 | 0.68 | 1.23 | 1.43 | 2.18 | 0 | 0 | 2.66 | 0 |
| VAR-B118 | 0 | 0 | 0.09 | 1.15 | 1.44 | 0.71 | 0 | 0 | 0 | 7.96 |
| VAR-B27 | 2.61 | 0 | 0.77 | 0.74 | 1.03 | 0.68 | 0 | 0 | 9.89 | 0 |
| VAR-B29 | 4.77 | 0 | 1.27 | 1.35 | 1.76 | 0.62 | 0 | 2.68 | 0 | 0 |
| VAR-B4 | 4.92 | 0 | 0.44 | 1.11 | 0.28 | 1.2 | 12 | 5.15 | 0 | 0 |
| VAR-B7 | 0 | 0 | 1.15 | 1.34 | 0.8 | 1.93 | 0 | 0 | 0 | 0 |
| VAR-B12 | 0 | 0 | 0.62 | 0.64 | 0.53 | 1.38 | 0 | 5.93 | 0 | 0 |
| VAR-B13 | 0 | 0 | 0.71 | 1.69 | 2.44 | 0 | 0 | 0 | 0 | 0 |
| VAR-B15 | 0 | 0 | 1.95 | 1.02 | 0.85 | 0 | 0 | 0 | 0 | 0 |
| VAR-B16 | 0 | 6.06 | 0.63 | 0.66 | 0.8 | 0 | 0 | 0 | 0 | 0 |
| VAR-B18 | 0 | 0 | 0.54 | 0 | 2.06 | 0 | 0 | 0 | 0 | 0 |
| VAR-B19 | 0 | 0 | 0.78 | 0.93 | 0.99 | 0 | 0 | 0 | 0 | 0 |
| VAR-B20 | 8.12 | 0 | 0.48 | 1.1 | 1.84 | 3.57 | 0 | 0 | 0 | 0 |
| VAR-B22 | 0 | 0 | 0.36 | 0.33 | 0.68 | 0 | 0 | 0 | 0 | 0 |
| VAR-B26 | 0 | 0 | 0.85 | 0.86 | 0.43 | 0 | 0 | 0 | 0 | 0 |
| VAR-B36 | 0 | 0 | 0.1 | 0 | 0.79 | 2.79 | 0 | 0 | 0 | 0 |
| VAR-B37 | 0 | 10.25 | 1.66 | 1.69 | 0.64 | 0 | 0 | 0 | 0 | 0 |
| VAR-B38 | 0 | 0 | 0.31 | 0.16 | 1.18 | 2.1 | 0 | 0 | 0 | 0 |
| VAR-B42 | 2.9 | 2.47 | 0.56 | 0.68 | 2.63 | 2.63 | 0 | 0 | 0 | 9.83 |
| VAR-B45 | 6.29 | 0 | 0.74 | 0.21 | 1.07 | 0 | 0 | 0 | 0 | 0 |
| VAR-B48 | 0 | 1.48 | 0.5 | 0.69 | 1.11 | 0 | 0 | 6.78 | 0 | 0 |
| VAR-B52 | 0 | 0 | 0.51 | 0.99 | 1.18 | 0 | 0 | 0 | 0 | 0 |
| VAR-B56 | 2.93 | 0 | 1.04 | 0.74 | 0.66 | 0 | 0 | 5.16 | 0 | 0 |
| VAR-B60 | 0 | 0 | 1.69 | 0.94 | 5.18 | 0 | 0 | 0 | 0 | 0 |
| VAR-B63 | 0 | 0 | 0.67 | 0.83 | 0.51 | 1.2 | 0 | 5.14 | 0 | 0 |
| VAR-B64 | 0 | 0 | 1.37 | 0 | 1.75 | 0 | 0 | 0 | 0 | 0 |
| VAR-B66 | 0 | 2.63 | 0.22 | 0.58 | 1.11 | 0 | 0 | 4.02 | 0 | 0 |
| VAR-B84 | 0 | 0 | 0.26 | 0 | 0 | 0 | 0 | 18.3 | 0 | 0 |
| VAR-B91 | 0 | 0 | 0.18 | 0 | 0.67 | 12.41 | 0 | 0 | 0 | 0 |
| VAR-B92 | 0 | 0 | 0.51 | 0.81 | 0.65 | 0 | 0 | 0 | 0 | 0 |
| VAR-B94 | 0 | 0 | 0.66 | 0.38 | 0 | 4.99 | 0 | 0 | 0 | 0 |
| VAR-B106 | 0 | 0 | 0.15 | 0.13 | 1.78 | 5.05 | 12.78 | 7.23 | 19.87 | 0 |
| VAR-B111 | 0 | 0 | 0 | 0.5 | 1.26 | 2.16 | 0 | 0 | 0 | 0 |
| VAR-B113 | 0 | 0 | 1.24 | 0.26 | 0.59 | 0 | 0 | 0 | 0 | 0 |
| VAR-B117 | 0 | 0 | 0.5 | 1.53 | 1.6 | 0 | 0 | 0 | 0 | 0 |
| VAR-B126 | 0 | 0 | 0.38 | 0.84 | 0 | 0 | 0 | 0 | 0 | 0 |

### 7.5. Example 5: High-Throughput AAV9 Library #2 Evaluation in the CNS

### 7.5.1. Overview

A library of 1E5 capsid variants of wild-type AAV9 (“Library #2”) was designed with the goals of producing a capsid that would package into AAV particles, transduce central nervous system tissues after intravenous injection with high efficiency, and de-target the liver and other tissue types. This library was designed and synthesized according to Example 1, but a subset of these variants included in this library included at least 3 of the mutations of VAR-1B1. In addition, the library contained a selection of variants which, relative to wild-type AAV9, included up to 12 amino acid mutations in addition to the mutations of the mutation set of VAR-B1. Without being bound by theory, these additional variants were designed to provide evidence confirming the enhanced performance of virus particles comprising the mutation sets described herein, for example, of VAR-B1, in different sequence contexts. Second, the library was cloned twice using two different versions of the viral genome, one with expression of the capsid gene under the control of a ubiquitous CBh promoter and the other with expression of the capsid gene under the control of a neuronal specific hSyn promoter. Each plasmid backbone contained a unique genomic identifier enabling analysis of biodistribution and transduction efficiencies of each capsid variant via each of the two promoters.

### 7.5.2. Materials and Methods

### 7.5.2.1. Library #2 Creation

Using machine learning algorithms trained on data from hundreds of thousands of capsid variants across multiple serotypes, including AAV particle production efficiency, and transduction and biodistribution across multiple tissues, a library of 1E5 capsid variants of wild-type AAV9 was designed with the goals of producing a capsid that would package into AAV particles, transduce central nervous system tissues after intravenous injection with high efficiency, and de-target the liver and other tissue types. This library included machine-guided design of variants with some mutation set sequence identity to VAR-B1. For example, variants were designed which preserved 3, 4, 5 or 6 of the mutations present in VAR-B1 and/or which included different amino acid mutations at 3, 4, 5, 6 or 7 of the positions of mutation present in VAR-B1. Among this set, many of the designed variants included one or more additional mutations (for example, 1, 2, 3, 4, 5, 6, 7, 8, 9, 10 or more) at positions other than those mutated in VAR-B1, and which machine learning algorithms trained on virus packaging and in vivo NHP tissue transduction and biodistribution data from a previous library of AAV9 variants, among other training sources, determined would impart the resulting variants with the favorable properties described above.

The designed capsid polypeptides were cloned into plasmids to create a library of plasmids encoding the capsid variants. A library of AAV variant genomes encoding each variant's capsid and a unique capsid variant barcode identifier was cloned into two ITR plasmid backbones as described previously (Ogden et al., 2019, Science 366 (6469):1139-1143, doi: 10.1126/science.aaw2900, hereby incorporated by reference in its entirety), one with expression of the capsid gene under the control of a neuronal specific human synuclein promoter (hSYN) and the other with expression of the capsid gene under the control of a ubiquitous CBh promoter. CBh is a ubiquitous promoter that has been shown to drive expression in CNS and other tissues. Each plasmid backbone contained a unique genomic identifier (“backbone tag”) enabling analysis of biodistribution and transduction efficiencies via each of the two promoters. Each capsid polypeptide variant was included in combination with between 1-500 unique genomic identifiers (“barcodes”) to enable measurement of biological replicates for each virus comprising a unique capsid polypeptide. Plasmids containing wild-type AAV9 as well as other AAV capsids described in the literature as purportedly targeting the CNS were also constructed, and included as controls. A library of AAV capsid variants, each comprising a genome encoding that variant's capsid polypeptides, was produced via transient triple transfection of adherent HEK293T. Transfections were completed at a 1:1:1/150 ratio of Helper, Rep, and VAR-inside-ITR plasmid, which has been optimized with these plasmids to limit cross packaging. Cells were harvested, lysed, and purified by a sequence of steps: (1) diafiltration, (2) tangential flow filtration (TFF), (3) iodixanol gradient purification, and (4) buffer exchange. The produced virus was tested for suitable sterility and endotoxin, and titer performed by ddPCR.

### 7.5.2.2. In Vitro Evaluation of Library

Data was prepared as described below. To measure each variant's packaging efficiency (or “production”), barcodes from vector genomes in the plasmid library and produced AAV library were prepared for Illumina sequencing using two rounds of PCR. Production efficiency for each of the produced AAV particle variants was normalized for its abundance in the input plasmid library, and was expressed by comparing barcode sequencing levels for each variant in the produced AAV virus particle vector pool to the barcode sequence levels for each variant in the input plasmid library used to create the AAV vector pool. The measurements of variant frequency in the AAV vector library also enable downstream normalization of biodistribution and transduction measurements by variant frequency in the input vector library.

### 7.5.2.3. In Vivo Evaluation of Library in Non-Human Primate

All NHP experiments were conducted in accordance with institutional policies and NIH guidelines. Two female Cynomolgus Macaque primates weighing 2.5 and 3.4 kg and seronegative for anti-AAV9 neutralizing antibodies (serum NAb titers <1:4 based on in vitro NAb assay) were selected for the study. Animals were treated with Methylprednisolone (40 or 80 mg IM) on Day −8 and weekly for the duration of the study. Prior to test article administration, samples of blood were collected. The animals received an intravenous injection of a mixture of the promoter vector libraries (total combined dose for each animal: 8.64 e13 vg/kg). During the in-life period the animals were monitored according to the animal facility's SOPs. Serum samples were collected at 2 days, 4 days, 7 days, and weekly after the injections. The animals were sacrificed 4 weeks after the injections and tissues were collected for biodistribution and transduction analyses. The tissues collected were the same as those shown in Table 14 above. All samples were collected into RNAlater® (Sigma-Aldrich) and incubated overnight at 4° C., after which the RNAlater® was drained and samples were frozen at −80° C. or maintained at 4° C. in RNAlater® (small subset of samples). In addition, samples of serum and cerebrospinal fluid were collected at necropsy and stored at −80° C.

Brain slices were dissected to isolate regions including, but not limited to, frontal cortex, temporal cortex, motor cortex, hippocampus, basal ganglia, midbrain, brainstem, and cerebellum. For all biodistribution and transduction analyses, total DNA and RNA was extracted from tissue samples with Trizol/chloroform and isopropanol precipitation. Reverse transcription was done with Protoscript II Reverse Transcriptase (NEB) utilizing primers that were specific to the vector transgene and included unique molecular identifiers (UMIs). Control reactions lacking the reverse transcriptase enzyme (-RT control) were also prepared. Quantification of biodistribution (based on viral DNA quantification) and transduction (based on viral transcript RNA quantification) was done with Luna Universal Probe qPCR Master Mix (NEB) using primers and probes specific to the transgene construct. Finally, samples were prepared for next-generation sequencing by amplifying the transgene barcode regions with primers compatible with Illumina NGS platform and sequenced with NextSeq 550 (Illumina).

After sequencing, the barcode tags were extracted from reads with the expected amplicon structure, and the abundance (number of reads or number of UMIs) of each barcode was recorded. Analyses were restricted to the set of barcodes that were present in the input plasmid sample, as measured by a separate sequencing assay that targeted the variant regions of the input plasmid sample.

To aggregate packaging replicates, the read counts from replicate virus production samples were summed. To aggregate biodistribution samples, read counts of viral DNA samples from the same tissue were summed. To aggregate transduction samples, the number of transduction events (measured by unique id tags detected from cDNA generated from RNA isolated from tissue) from samples from the same tissue were summed.

Virus packaging was calculated by normalizing aggregated production replicates with input plasmid abundance. Biodistribution and transduction of tissue were calculated by normalizing aggregated biodistribution or transduction samples with input virus abundance. The output was reported as a fold change relative to the rates observed for wild-type AAV9.

### 7.5.3. Results

Analysis of the production, transduction and biodistribution results across multiple brain regions (including aggregated reads from all brain region collected) from both the CBH-driven and hSyn-driven constructs identified over 7100 unique capsid sequences which exhibited aggregate brain transduction better than wt AAV9, and which contained at least 3 of the mutations of the mutation set of VAR-B1. Of this set, over 1850 unique variants exhibited aggregate brain transduction at least 5× better than wtAAV9, of which, 770 unique variants exhibited aggregate brain transduction at least 20× better than wtAAV9. Of these, 119 of these variants exhibited aggregate brain transduction at least 90× better than wtAAV9. From amongst this set, VAR-1 to VAR-62 were identified as having particularly relevant properties for use in CNS-targeting gene therapy applications, including high brain transduction, low liver biodistribution and good productivity from the HEK293 triple-transfection based process described above relative to wild-type AAV9. The capsid polypeptide VP1 sequences of select variants from this library are provided as SEQ ID NOs: 12 to 73 (corresponding representative nucleic acid sequences are provided in SEQ ID NOS: 74 to 135) as shown in Table 37. The mutation set associated with each of these variants is also provided in Table 27. The production, biodistribution and transduction results for VAR-1 to VAR-62 this library experiment are summarized in Table 21 (production), Tables 22-1 and 22-2 (CNS biodistribution), Table 23 (spinal cord and periphery biodistribution), Tables 24-1 and 24-2 (CNS transduction) and Table 25 (spinal cord and periphery transduction). In addition, a “profile” score was generated which ranked each capsid for its performance across a variety of properties. Table 26 summarizes the profile score for VAR-1 to VAR-62, and was calculated using a 70/20/10 ratio of relative (to virus particles comprising wild-type AAV9 capsids) performance of aggregated brain transduction as a measure of tissue targeting (higher values preferred; aggregated across all brain tissues collected and across transduction from both the CBH and hSYN promoters), average biodistribution across liver, spleen, and DRG as a measure of tissue detargeting (lower relative values preferred), and production rate (as a measure of manufacturability; higher values preferred), respectively. In addition, capsid variants only received a profile score if they packaged virus particles at least 17% as well as wtAAV9 (above the cross-packaging rate of negative controls). Profile scores are shown in Table 26.

TABLE 21
Production efficiency of variant virus particles relative
to production efficiency of WT AAV9 virus particles, in
each case comprising genomes with the cap gene under the
control of the CBh promoter, in HEK293 cells in vitro.

| | Fold Change Relative to |
|---|---|
| VAR-# | wtAAV9 |
| VAR-1 | 0.73 |
| VAR-2 | 0.38 |
| VAR-3 | 0.68 |
| VAR-4 | 0.42 |
| VAR-5 | 0.36 |
| VAR-6 | 0.6 |
| VAR-7 | 0.61 |
| VAR-8 | 0.51 |
| VAR-9 | 0.65 |
| VAR-10 | 0.42 |
| VAR-11 | 0.81 |
| VAR-12 | 0.49 |
| VAR-13 | 0.44 |
| VAR-14 | 0.51 |
| VAR-15 | 0.55 |
| VAR-16 | 0.6 |
| VAR-17 | 0.6 |
| VAR-18 | 0.43 |
| VAR-19 | 0.68 |
| VAR-20 | 0.56 |
| VAR-21 | 0.14 |
| VAR-22 | 0.68 |
| VAR-23 | 0.42 |
| VAR-24 | 0.48 |
| VAR-25 | 0.62 |
| VAR-26 | 0.47 |
| VAR-27 | 0.36 |
| VAR-28 | 0.31 |
| VAR-29 | 0.53 |
| VAR-30 | 0.28 |
| VAR-31 | 0.58 |
| VAR-32 | 0.76 |
| VAR-33 | 0.39 |
| VAR-34 | 0.58 |
| VAR-35 | 0.58 |
| VAR-36 | 0.51 |
| VAR-37 | 0.65 |
| VAR-38 | 0.56 |
| VAR-39 | 0.47 |
| VAR-40 | 0.7 |
| VAR-41 | 0.31 |
| VAR-42 | 0.47 |
| VAR-43 | 0.58 |
| VAR-44 | 0.78 |
| VAR-45 | 0.44 |
| VAR-46 | 0.34 |
| VAR-47 | 0.15 |
| VAR-48 | 0.48 |
| VAR-49 | 0.53 |
| VAR-50 | 0.64 |
| VAR-51 | 0.46 |
| VAR-52 | 0.52 |
| VAR-53 | 0.37 |
| VAR-54 | 0.47 |
| VAR-55 | 0.53 |
| VAR-56 | 0.6 |
| VAR-57 | 0.54 |
| VAR-58 | 0.23 |
| VAR-59 | 0.69 |
| VAR-60 | 0.47 |
| VAR-61 | 0.52 |
| VAR-62 | 0.77 |

TABLE 22
1. Fold change in biodistribution of virus particles comprising the indicated variant capsid polypeptide, relative to the biodistribution of virus particles comprising WT AAV9
for brain regions, with the 95% confidence interval width shown in parentheses following each value. “CBh indicates measurements from virus particles comprising genomes
with the cap gene under the control of the CBh promoter. hSyn indicates virus particles comprising genomes with the cap gene under the control of the hSyn promoter. “Aggregated”
in relation to a tissue is an average across all measured areas in the specified organ. “Aggregated” in relation to a promoter is an average across the CBh and hSyn promoters.

| | ORGAN | | | | | | | | | | | |
|---|---|---|---|---|---|---|---|---|---|---|---|---|
| | Brain | Brain | Brain | Brain | Brain | Brain | Brain | Brain | Brain | Brain | Brain | Brain |
| | TISSUE | | | | | | | | | | | |
| | | | | Basal | Basal | Basal | | | | | | |
| | Aggregated | Aggregated | Aggregated | Ganglia | Ganglia | Ganglia | Brain Stem | Brain Stem | Brain Stem | Cerebellum | Cerebellum | Cerebellum |
| | PROMOTER | | | | | | | | | | | |
| VAR# | CBh | hSyn | Aggregated | CBh | hSyn | Aggregated | CBh | hSyn | Aggregated | CBh | hSyn | Aggregated |
| VAR-1 | 40.71 | 32.84 | 36.27 | 49.58 | 28.34 | 42.16 | 25.21 | 18.22 | 22.53 | 56.01 | 55.41 | 50.81 |
| | (8.17) | (16.43) | (7.38) | (19.19) | (36.03) | (17.53) | (10.78) | (24.38) | (10.74) | (25.82) | (52.68) | (24.51) |
| VAR-2 | 48.72 | 36.66 | 47.84 | 59.38 | 44.13 | 58.19 | 31.53 | 28.8 | 33.05 | 69.85 | 38.82 | 61.88 |
| | (9.9) | (9.24) | (8.71) | (36.97) | (25.25) | (20.91) | (14.47) | (19.29) | (14.28) | (32.42) | (28.55) | (28.12) |
| VAR-3 | 43.72 | 34.62 | 40.19 | 45.89 | 44.85 | 44.16 | 28.12 | 25.32 | 26.76 | 69.36 | 42.33 | 60.06 |
| | (8.76) | (13.46) | (8.16) | (19.15) | (31.38) | (19.95) | (11.81) | (20.87) | (10.42) | (31.21) | (39.43) | (28.05) |
| VAR-4 | 35.28 | 30.76 | 33.18 | 37.24 | 43.61 | 37.75 | 27.79 | 22.41 | 25.9 | 50.34 | 40.15 | 46.09 |
| | (6.13) | (9.83) | (5.74) | (17.73) | (24.54) | (14.16) | (9.22) | (17.73) | (8.94) | (17.98) | (25.15) | (14.73) |
| VAR-5 | 32.06 | 31.69 | 32.61 | 42.35 | 41.82 | 43.1 | 20.52 | 32.92 | 24.78 | 42.92 | 35.95 | 41.45 |
| | (6.76) | (9.26) | (5.83) | (16.93) | (24.5) | (17.13) | (8.81) | (17.69) | (10.23) | (25.99) | (28.41) | (21.16) |
| VAR-6 | 35.19 | 29.91 | 34.18 | 41.46 | 34.12 | 39.87 | 27.22 | 16.54 | 24.35 | 42.66 | 36.48 | 41.51 |
| | (5.94) | (6.86) | (4.86) | (16.09) | (16.61) | (13.35) | (7.98) | (12.21) | (7.17) | (16.1) | (15.36) | (13.84) |
| VAR-7 | 35.62 | 30.56 | 33.43 | 44.27 | 32.49 | 40.04 | 24.02 | 17.99 | 22.09 | 48.8 | 44.64 | 46.26 |
| | (4.08) | (7.32) | (3.21) | (12.22) | (21.88) | (11.67) | (5.42) | (12.36) | (7.53) | (12.43) | (32.48) | (1.2) |
| VAR-8 | 54.86 | 40.39 | 51.37 | 70.42 | 49.33 | 64.98 | 38.63 | 34.7 | 38.3 | 58.45 | 44.66 | 55.24 |
| | (10.81) | (10.54) | (8.09) | (28.76) | (34.46) | (24.02) | (12.8) | (23.02) | (12.81) | (31.47) | (37.42) | (25.25) |
| VAR-9 | 33.02 | 30.02 | 32.93 | 39.84 | 29.6 | 37.37 | 26.91 | 23.54 | 26.47 | 40.26 | 30.78 | 38.05 |
| | (6.17) | (9.77) | (5.6) | (14.71) | (18.81) | (15.88) | (9.45) | (13.25) | (7.66) | (21.02) | (19.66) | (19.3) |
| VAR-10 | 36.92 | 33.56 | 38.58 | 47.98 | 35.52 | 46.49 | 26.19 | 19.25 | 25.13 | 40.92 | 48.82 | 48.8 |
| | (8.18) | (7.65) | (7.02) | (19.84) | (19.94) | (16.23) | (11.92) | (13.37) | (8.4) | (27.22) | (28.82) | (18.49) |
| VAR-11 | 33.57 | 19.27 | 28.64 | 43.03 | 16.81 | 35.49 | 18.24 | 20.9 | 17.02 | 48.81 | 17.6 | 39.59 |
| | (8.16) | (12.84) | (7.24) | (19.74) | (39.06) | (14.41) | (9.52) | (34.85) | (8.48) | (23.8) | (45.96) | (21.71) |
| VAR-12 | 31.47 | 29.97 | 31.12 | 39.5 | 37.31 | 38.95 | 23.34 | 22.77 | 23.25 | 39.86 | 39.75 | 39.98 |
| | (6.41) | (9.58) | (5.81) | (17.62) | (25.03) | (13.31) | (11.08) | (14.72) | (8.31) | (21.09) | (32.66) | (18.97) |
| VAR-13 | 37.22 | 33.67 | 38.98 | 47.79 | 38.59 | 48 | 27.75 | 22.07 | 27.42 | 46.65 | 44.5 | 50.45 |
| | (6.09) | (6.31) | (5.85) | (16.12) | (15.49) | (11.85) | (8.91) | (1.41) | (7.52) | (23.04) | (14.53) | (19.3) |
| VAR-14 | 49.03 | 39.95 | 51.76 | 60.73 | 56.16 | 68.72 | 28.65 | 27.37 | 32.03 | 81.73 | 54.04 | 79.38 |
| | (12.85) | (11.03) | (12.14) | (40.13) | (30.29) | (28.73) | (14.46) | (14.78) | (12.82) | (45.57) | (30.06) | (35.71) |
| VAR-15 | 45.48 | 42.57 | 44.29 | 50.25 | 48.29 | 49.25 | 28.76 | 29.75 | 28.83 | 58.66 | 48.19 | 54.99 |
| | (4.65) | (9.31) | (4.5) | (13.24) | (16.28) | (10.47) | (8.89) | (13.32) | (5.41) | (16.12) | (9.83) | (17.64) |
| VAR-16 | 20.52 | 16.59 | 20.02 | 29.85 | 16.64 | 26.13 | 16.08 | 12.17 | 15.34 | 24.04 | 24.97 | 25.82 |
| | (4.5) | (4.84) | (3.72) | (14.63) | (11.7) | (10.6) | (5.94) | (8.48) | (5.42) | (12.65) | (16.98) | (13.08) |
| VAR-17 | 41.92 | 29.14 | 38.03 | 54.39 | 33.45 | 47.91 | 24.3 | 22.63 | 23.76 | 46 | 28.97 | 40.54 |
| | (8.05) | (10.41) | (6.29) | (20.13) | (25.94) | (18.58) | (9.25) | (18.24) | (9.61) | (22.74) | (26.99) | (18.53) |
| VAR-18 | 40.38 | 43.51 | 47.4 | 46.23 | 55.16 | 57.63 | 26.62 | 31.75 | 32.19 | 53.74 | 56.02 | 63.07 |
| | (6.74) | (5.16) | (6.4) | (20.91) | (22.34) | (19.21) | (12.57) | (11.11) | (10.92) | (26.57) | (3.33) | (20.5) |
| VAR-19 | 31.61 | 29.34 | 29.59 | 33.77 | 26.31 | 30.45 | 26.32 | 21.29 | 24.26 | 38.25 | 40.57 | 36.63 |
| | (5.2) | (10.76) | (5.67) | (17.77) | (19.46) | (11.76) | (6.83) | (16.49) | (8.15) | (21.19) | (34.85) | (14.38) |
| VAR-20 | 34.33 | 33.7 | 34.13 | 37.55 | 47.99 | 40.72 | 25.44 | 20.6 | 24.09 | 36.47 | 41.31 | 37.98 |
| | (8.06) | (12.07) | (6.13) | (19.21) | (28.72) | (17.11) | (10.9) | (18.66) | (10.36) | (24.6) | (37.76) | (19.54) |
| VAR-21 | 24.22 | 16.45 | 22.6 | 30.4 | 15.53 | 26.11 | 26.1 | 17.68 | 24.3 | 12.91 | 24.35 | 19.21 |
| | (13.28) | (14.26) | (10.18) | (31.2) | (37.63) | (27) | (25.09) | (30.66) | (17.65) | (31.2) | (46.83) | (28.54) |
| VAR-22 | 35.4 | 27.19 | 32.49 | 42.89 | 29.7 | 38.4 | 20.63 | 24.62 | 21.18 | 45.66 | 31.91 | 40.75 |
| | (5.81) | (6.34) | (4.11) | (11.25) | (18.3) | (12.72) | (6.56) | (12.77) | (3.98) | (14.18) | (20.3) | (14.66) |
| VAR-23 | 51.24 | 42.14 | 49.87 | 64.3 | 39.38 | 57.47 | 40.06 | 33.03 | 38.95 | 57.8 | 45.28 | 55.43 |
| | (13.01) | (16.08) | (11.89) | (34.88) | (34.22) | (28.99) | (21.37) | (27.55) | (17.14) | (34.85) | (51.63) | (31.33) |
| VAR-24 | 43.4 | 37.13 | 43.75 | 53.97 | 47.25 | 55.01 | 24.42 | 25.76 | 26.4 | 63.54 | 46.14 | 60.67 |
| | (1.41) | (6.96) | (1.22) | (14.39) | (17.56) | (4.5) | (6.03) | (9.84) | (0.69) | (14.35) | (24.49) | (10.81) |
| VAR-25 | 42.23 | 33.61 | 41.56 | 48.71 | 41.37 | 49.18 | 31.64 | 21.76 | 29.59 | 56.25 | 31.39 | 49.44 |
| | (1.37) | (5.14) | (5.88) | (19.42) | (21.15) | (18.39) | (8.14) | (10.23) | (6.46) | (18.46) | (16.57) | (11.79) |
| VAR-26 | 66.06 | 59.17 | 64.59 | 89.49 | 69.42 | 83.97 | 49.17 | 36.43 | 45.84 | 76.41 | 75.07 | 76.91 |
| | (13.73) | (14.12) | (11.06) | (37.36) | (40.82) | (28.24) | (15.67) | (23.19) | (16.1) | (26.59) | (60.7) | (33.51) |
| VAR-27 | 38.84 | 23.76 | 35.62 | 45.82 | 21.59 | 38.65 | 24.64 | 17.34 | 23.59 | 49.88 | 29.12 | 45.17 |
| | (8.41) | (5.82) | (6.59) | (20.34) | (18.2) | (15.25) | (11.17) | (10.56) | (9.23) | (23.04) | (23.36) | (20.69) |
| VAR-28 | 34.62 | 33.05 | 40.7 | 44.15 | 37.67 | 49.23 | 21.77 | 31.53 | 31.8 | 39.55 | 39.71 | 48.93 |
| | (8.87) | (4.78) | (7.23) | (20.5) | (2.04) | (18.95) | (11.51) | (1.56) | (10.43) | (28.18) | (18.4) | (19.85) |
| VAR-29 | 20.46 | 21.5 | 22.87 | 24.08 | 23.74 | 26.3 | 23.5 | 18 | 22.94 | 24.6 | 34.77 | 32.33 |
| | (5.29) | (5.52) | (4.95) | (12.7) | (16.03) | (11.17) | (10.05) | (11.25) | (7.4) | (19.51) | (17.37) | (14.91) |
| VAR-30 | 60.37 | 47.74 | 61.07 | 88.1 | 60.89 | 84.75 | 33.84 | 30.93 | 35.9 | 86.39 | 57.43 | 82.17 |
| | (10.12) | (11.35) | (8.94) | (34.86) | (35.5) | (26.09) | (13.08) | (13.39) | (13.5) | (44.16) | (23.35) | (30.96) |
| VAR-31 | 46.11 | 41.92 | 45.54 | 57.25 | 52.81 | 56.82 | 32.48 | 36.79 | 34.3 | 60.2 | 46.05 | 56.45 |
| | (6.28) | (8.87) | (6.45) | (22.98) | (18.51) | (14.45) | (8.09) | (19.25) | (11.59) | (16.92) | (24.74) | (22.37) |
| VAR-32 | 35.35 | 33.83 | 35.14 | 43.8 | 37.98 | 42.29 | 22.7 | 21.78 | 22.58 | 47.57 | 47.24 | 47.87 |
| | (10.15) | (13.95) | (9.27) | (28.69) | (34.77) | (25.39) | (12.83) | (20.02) | (11.88) | (31.7) | (49.71) | (32.18) |
| VAR-33 | 53.92 | 32.84 | 48.8 | 65.74 | 37.4 | 58.29 | 37.8 | 23.11 | 34.23 | 67.51 | 33.61 | 57.55 |
| | (5.29) | (6.89) | (4.68) | (10.95) | (21.11) | (1.61) | (5.95) | (7.14) | (7.81) | (18.3) | (12.82) | (1.43) |
| VAR-34 | 34.46 | 31.13 | 33.37 | 48.89 | 35.52 | 44.71 | 23.51 | 33.73 | 26.21 | 45.19 | 39.44 | 43.23 |
| | (6.5) | (9.13) | (4.92) | (19.75) | (19.4) | (13.83) | (7.65) | (15.77) | (6.78) | (24.62) | (24.92) | (14.67) |
| VAR-35 | 19.85 | 16.36 | 19.76 | 22.94 | 18.77 | 22.83 | 9.34 | 11.88 | 10.93 | 22.63 | 18.85 | 22.73 |
| | (5.1) | (6.5) | (4.56) | (12.57) | (17) | (10.99) | (6.74) | (10.81) | (5.75) | (19.36) | (17.6) | (12.76) |
| VAR-36 | 27.43 | 23.72 | 25.92 | 35.38 | 31.2 | 33.52 | 15.08 | 11.8 | 14.03 | 35.46 | 45.7 | 37.49 |
| | (7.34) | (10.08) | (5.79) | (17.45) | (27.88) | (13.35) | (10.13) | (15.1) | (7.43) | (21.34) | (35.55) | (18.55) |
| VAR-37 | 31.16 | 30.18 | 31.33 | 33.7 | 32.84 | 33.97 | 24.74 | 17.44 | 22.84 | 41.3 | 40.64 | 41.8 |
| | (4.54) | (6.95) | (4.6) | (10.54) | (13.98) | (11.01) | (6.3) | (8.61) | (6.28) | (9.34) | (2.49) | (9.86) |
| VAR-38 | 37.21 | 32.46 | 39.41 | 43.2 | 42.12 | 48.42 | 24.63 | 21.98 | 25.96 | 56.82 | 39.76 | 55.33 |
| | (0.69) | (0.99) | (0.7) | (0.76) | (1.28) | (0.78) | (4.41) | (0.68) | (0.44) | (1.13) | (1.25) | (1.01) |
| VAR-39 | 28.98 | 24.89 | 30.19 | 33.78 | 27.53 | 34.56 | 25.28 | 21.29 | 25.81 | 29.47 | 26.36 | 31.59 |
| | (5.58) | (4.51) | (4.69) | (15.52) | (14.85) | (13.88) | (7.49) | (8.83) | (6.09) | (17.84) | (15.22) | (14.34) |
| VAR-40 | 57.33 | 52.96 | 59.23 | 72.81 | 63.12 | 73.65 | 38.73 | 33.39 | 38.78 | 75.78 | 70.12 | 78.87 |
| | (1.89) | (3.4) | (1.8) | (15.72) | (18.53) | (13.27) | (12.76) | (13.27) | (3.92) | (16.04) | (22.13) | (8.58) |
| VAR-41 | 40.96 | 26.81 | 37.68 | 46.46 | 27.78 | 41.6 | 35.6 | 19.32 | 31.19 | 56.8 | 26.87 | 47.67 |
| | (8.48) | (8.28) | (8.68) | (21.92) | (23.54) | (16.12) | (14.38) | (14.36) | (13.17) | (36.38) | (23.94) | (29.72) |
| VAR-42 | 40.62 | 51.82 | 44.16 | 45.88 | 74.79 | 55.04 | 24.05 | 38.89 | 28.32 | 53.74 | 38.39 | 48.98 |
| | (11.74) | (18.1) | (10.58) | (27.16) | (44.45) | (26.13) | (15.29) | (29) | (14.74) | (36.54) | (31.78) | (30.15) |
| VAR-43 | 52.63 | 43.99 | 50.61 | 67.58 | 51.45 | 63.26 | 36.82 | 35.56 | 36.9 | 56.65 | 35.8 | 50.4 |
| | (7.63) | (11.22) | (8.2) | (23.11) | (24.49) | (15.25) | (8.09) | (13.3) | (9.39) | (28.94) | (29.05) | (18.78) |
| VAR-44 | 39.82 | 32.97 | 39.86 | 47.76 | 37.29 | 46.89 | 30.13 | 25.45 | 30.17 | 51.3 | 40.84 | 50.87 |
| | (4.04) | (4.03) | (0.83) | (1.2) | (14.59) | (9.48) | (0.84) | (6.84) | (5.09) | (7.84) | (13.06) | (11.63) |
| VAR-45 | 50.3 | 44.99 | 47.37 | 57.23 | 68.44 | 57.92 | 33.1 | 37.04 | 33.03 | 68.83 | 69.43 | 66.37 |
| | (13.71) | (23.12) | (9.77) | (32.13) | (58.44) | (26.81) | (18.45) | (36.83) | (14.74) | (49.64) | (60.88) | (39.05) |
| VAR-46 | 37.28 | 31.01 | 35.89 | 46.35 | 26.55 | 40.51 | 27.91 | 20.43 | 26.03 | 52.43 | 43.34 | 50.34 |
| | (6.66) | (8.21) | (6.54) | (19.04) | (20.19) | (14.6) | (11.57) | (15.71) | (8.91) | (23.99) | (29.21) | (17.21) |
| VAR-47 | 54.26 | 41.85 | 53.31 | 73.47 | 71.81 | 79.54 | 31.25 | 20.02 | 28.74 | 80.96 | 39.64 | 68.77 |
| | (15.56) | (15.22) | (11.74) | (40.11) | (41.14) | (34.07) | (16.88) | (18.45) | (14.69) | (50.44) | (39.64) | (32.71) |
| VAR-48 | 37.36 | 28.14 | 34.85 | 47.54 | 39.73 | 45.57 | 24.32 | 24.28 | 24.55 | 42.92 | 33.4 | 40.27 |
| | (10.02) | (12.31) | (8.41) | (21.65) | (31.8) | (17.37) | (13.44) | (21.32) | (9.69) | (33.49) | (28.01) | (23.7) |
| VAR-49 | 21.8 | 21.82 | 21.51 | 24.77 | 38.83 | 28.34 | 14.37 | 16.61 | 14.78 | 24.56 | 24.64 | 24.2 |
| | (6.16) | (11.34) | (6.24) | (20.7) | (44.16) | (19.55) | (8.95) | (17.62) | (9.29) | (22.45) | (38.85) | (18.51) |
| VAR-50 | 41.36 | 36.09 | 44.98 | 43.95 | 48.12 | 54.19 | 26.31 | 25.66 | 29.61 | 56.58 | 40.81 | 57.01 |
| | (5.13) | (1.59) | (3.82) | (17.27) | (2.02) | (9.68) | (7.38) | (0.98) | (3.84) | (14.93) | (1.75) | (13.61) |
| VAR-51 | 37.51 | 33.05 | 37.62 | 42.61 | 43.69 | 45.27 | 25.99 | 21.66 | 25.5 | 50.59 | 44.14 | 50.75 |
| | (4.13) | (1.56) | (2.95) | (9.2) | (13.44) | (7.73) | (0.7) | (8.47) | (5.96) | (6.98) | (11.32) | (11.97) |
| VAR-52 | 20.38 | 24.56 | 20.06 | 23.06 | 31.94 | 23.48 | 14.38 | 15.02 | 13.82 | 23.84 | 33.02 | 24.23 |
| | (4.62) | (9.97) | (4.4) | (13.35) | (31.66) | (9.97) | (7.09) | (16.37) | (6.8) | (14.62) | (36.21) | (13.04) |
| VAR-53 | 46.04 | 40.09 | 49.36 | 74.59 | 47.58 | 70.27 | 32.22 | 41.87 | 41.33 | 50.86 | 39.08 | 52.14 |
| | (12.32) | (9.97) | (9.58) | (40.93) | (24.78) | (26.86) | (19.07) | (17.5) | (15.01) | (35.82) | (29.32) | (30.31) |
| VAR-54 | 13.72 | 7.91 | 12.4 | 16.37 | 7.94 | 13.95 | 8.39 | 5.9 | 8.1 | 10.88 | 11.36 | 12.95 |
| | (5.82) | (5.25) | (4.31) | (17.04) | (12.86) | (10.52) | (9.94) | (8.27) | (6.48) | (18.81) | (15.56) | (12.36) |
| VAR-55 | 38.05 | 32.46 | 37.7 | 46.74 | 49.63 | 50.24 | 27.12 | 21.03 | 26.02 | 47.54 | 30.45 | 43.1 |
| | (7.94) | (8.7) | (6.03) | (18.38) | (26.91) | (16.53) | (12.52) | (15.22) | (10.15) | (24.6) | (19.44) | (21.15) |
| VAR-56 | 21.23 | 23.7 | 23.18 | 24.67 | 34.52 | 29.79 | 13.46 | 15.06 | 14.57 | 28.06 | 26.79 | 29.03 |
| | (5.23) | (6.73) | (4.17) | (11.87) | (16.8) | (11.27) | (6.76) | (10.97) | (6.88) | (14.52) | (18.05) | (13.23) |
| VAR-57 | 20.79 | 24.66 | 24.36 | 26.48 | 24.84 | 28.12 | 13.9 | 24.13 | 19.29 | 31.02 | 27.72 | 32.44 |
| | (5.86) | (7.4) | (5.88) | (15.89) | (15.83) | (12.9) | (8.75) | (13.3) | (8.7) | (21.51) | (23.05) | (18.1) |
| VAR-58 | 17.95 | 16.56 | 20.92 | 19.9 | 25.14 | 28.36 | 15.31 | 9.33 | 14.21 | 23.98 | 22.85 | 29.12 |
| | (7) | (6.64) | (5.57) | (19.24) | (15.04) | (14.92) | (14.11) | (8.37) | (9.27) | (26.64) | (18.65) | (21.38) |
| VAR-59 | 33.56 | 29.65 | 32.97 | 43.68 | 34.67 | 41.57 | 27.16 | 28.19 | 27.99 | 39.22 | 34.97 | 38.67 |
| | (6.32) | (8.7) | (7.01) | (21.96) | (20.09) | (15.94) | (8.67) | (13.62) | (9.31) | (19.2) | (30.59) | (16.16) |
| VAR-60 | 21.09 | 18.04 | 19.16 | 26.13 | 22.49 | 23.64 | 13.8 | 15.32 | 13.26 | 27.79 | 15.23 | 23.38 |
| | (2.76) | (6.45) | (2.89) | (9.48) | (19.21) | (8.34) | (4.25) | (13.24) | (4.99) | (8.57) | (18.24) | (10.23) |
| VAR-61 | 21.59 | 23.58 | 23.73 | 27.28 | 30.62 | 30.45 | 9.86 | 14.94 | 12.32 | 28.4 | 28.04 | 30.21 |
| | (6) | (8.06) | (5.21) | (15.06) | (19.25) | (13.94) | (7.7) | (11.97) | (6.25) | (19.29) | (21.8) | (18.55) |
| VAR-62 | 27.17 | 26.4 | 26.97 | 34.91 | 28.82 | 33.08 | 16.89 | 17.84 | 17.18 | 30.4 | 33.41 | 31.41 |
| | (5.52) | (6.8) | (3.62) | (13.31) | (14.37) | (9.47) | (6.51) | (10.21) | (6.06) | (11.93) | (19.46) | (10.7) |

2. Fold change in biodistribution of virus particles comprising the indicated variant capsid polypeptide, relative to the biodistribution of virus particles comprising WT AAV9
for brain regions, with the 95% confidence interval width shown in parentheses following each value. CBh indicates measurements from virus particles comprising genomes
with the cap gene under the control of the CBh promoter. hSyn indicates virus particles comprising genomes with the cap gene under the control of the hSyn promoter. “Aggregated”
in relation to a tissue is an average across all measured areas in the specified organ. “Aggregated” in relation to a promoter is an average across the CBh and hSyn promoters.

| | ORGAN | | | | | | | | | | | |
|---|---|---|---|---|---|---|---|---|---|---|---|---|
| | Brain | Brain | Brain | Brain | Brain | Brain | Brain | Brain | Brain | Brain | Brain | Brain |
| | TISSUE | | | | | | | | | | | |
| | | | | Hippo- | Hippo- | Hippo- | | | | Temporal | Temporal | Temporal |
| | Forebrain | Forebrain | Forebrain | Campus | Campus | Campus | Midbrain | Midbrain | Midbrain | Cortex | Cortex | Cortex |
| | PROMOTER | | | | | | | | | | | |
| VAR-# | CBh | hSyn | Aggregated | CBh | hSyn | Aggregated | CBh | hSyn | Aggregated | CBh | hSyn | Aggregated |
| VAR-1 | 41.11 | 34.71 | 36.97 | 30.34 | 22.59 | 26.28 | 38.59 | 21.48 | 32.94 | 34.03 | 30.26 | 30.95 |
| | (15.57) | (31.41) | (12.36) | (16.53) | (27.67) | (11.66) | (20.85) | (42.96) | (19.07) | (12.83) | (27.18) | (10.97) |
| VAR-2 | 48.17 | 37.47 | 47.82 | 30.74 | 27.85 | 32.92 | 53.26 | 39.77 | 52.16 | 35.54 | 33.93 | 38.2 |
| | (25.36) | (20.29) | (17.36) | (22.63) | (19.56) | (19.2) | (32.08) | (30.89) | (24.64) | (15.87) | (17.71) | (14.11) |
| VAR-3 | 44.33 | 29.07 | 39.39 | 29.52 | 25.3 | 27.38 | 35.99 | 36.69 | 35.06 | 35.73 | 29.75 | 33.33 |
| | (16.34) | (21.11) | (12.69) | (19.21) | (28.84) | (17.51) | (24.29) | (44.28) | (21.62) | (12.72) | (20.04) | (12.07) |
| VAR-4 | 31.75 | 26.55 | 29.63 | 34.19 | 19.71 | 29.26 | 40.88 | 35.73 | 38.47 | 25.47 | 17.81 | 22.98 |
| | (10.64) | (16.41) | (8.87) | (14.82) | (22.73) | (13.42) | (19.78) | (34.23) | (17.78) | (9.65) | (15.42) | (7.84) |
| VAR-5 | 33.74 | 24.1 | 31.22 | 22.94 | 14.65 | 20.5 | 30.54 | 17.47 | 26.77 | 22.89 | 32.22 | 26.38 |
| | (14.02) | (17.74) | (11.89) | (13.35) | (15.25) | (9.47) | (19.74) | (28.85) | (17.01) | (9.35) | (19.43) | (9.08) |
| VAR-6 | 37.75 | 33.18 | 37.03 | 20.9 | 29.33 | 24.51 | 39.71 | 30 | 37.29 | 28.34 | 27.21 | 28.55 |
| | (11.37) | (13.09) | (12.25) | (12.37) | (23.61) | (11.36) | (18.93) | (25.79) | (13.41) | (9.93) | (15.31) | (10.03) |
| VAR-7 | 34.63 | 31.26 | 32.93 | 25.81 | 19.54 | 23.35 | 40.35 | 32.25 | 37.27 | 25.67 | 25.06 | 24.91 |
| | (11) | (14.43) | (5.19) | (8.56) | (15.61) | (11.68) | (1.07) | (23.07) | (16.28) | (5.8) | (13.28) | (7.62) |
| VAR-8 | 52.44 | 30.87 | 46.42 | 58.2 | 18.14 | 45.02 | 69.84 | 48.33 | 64.29 | 46.85 | 43.01 | 46.82 |
| | (17.85) | (18.65) | (14.71) | (23.69) | (19.3) | (20.2) | (32.58) | (38.17) | (20.98) | (19.58) | (21.3) | (19.77) |
| VAR-9 | 34.63 | 31.87 | 34.66 | 17.44 | 25.7 | 21.17 | 33.2 | 47.08 | 39.11 | 26.9 | 28.19 | 28.08 |
| | (12.72) | (14.63) | (14.04) | (13.22) | (27.39) | (12.25) | (19.81) | (26.92) | (19.64) | (11.46) | (15.48) | (8.46) |
| VAR-10 | 44.02 | 32.12 | 42.31 | 29.88 | 14.33 | 25.21 | 27.17 | 24.88 | 28.48 | 31.64 | 37.45 | 36.77 |
| | (19.83) | (14.42) | (16.53) | (21.28) | (19.69) | (16.62) | (23.93) | (26.23) | (17.3) | (12.64) | (17.71) | (12.69) |
| VAR-11 | 33.59 | 24.83 | 29.41 | 25.42 | 1.67 | 19.6 | 39.99 | 20.71 | 33.83 | 22.32 | 22.21 | 20.28 |
| | (14.96) | (51.24) | (10.56) | (14.25) | (3.09) | (13.87) | (26.77) | (54.85) | (18.83) | (10.64) | (34.87) | (8.46) |
| VAR-12 | 34.54 | 28.92 | 32.96 | 20.9 | 21.65 | 21.22 | 24.41 | 17.96 | 22.53 | 25.25 | 25.2 | 25.31 |
| | (13.19) | (18.9) | (11.52) | (13.75) | (13.85) | (11.57) | (17.88) | (29.54) | (15.2) | (9.83) | (17.29) | (8.61) |
| VAR-13 | 35.63 | 29.98 | 36.19 | 35.61 | 33.47 | 38.35 | 40.22 | 27.37 | 37.89 | 26.24 | 32.22 | 31.21 |
| | (11.37) | (11.92) | (10.61) | (18.23) | (13.02) | (11.23) | (17.87) | (13.76) | (12.59) | (8.49) | (11.63) | (10.46) |
| VAR-14 | 37.82 | 32.53 | 40.82 | 21.43 | 14.77 | 21.25 | 41.05 | 23.66 | 37.32 | 42.81 | 38.2 | 46.8 |
| | (22.62) | (18.45) | (19.37) | (21.78) | (17.81) | (17.8) | (40.91) | (32.47) | (23.44) | (23.43) | (18.18) | (15.81) |
| VAR-15 | 51.03 | 42.27 | 48.19 | 34.71 | 39.61 | 35.87 | 50.53 | 43.08 | 48.01 | 39.15 | 42.81 | 39.86 |
| | (11.02) | (14.86) | (11.88) | (21.73) | (18.4) | (12) | (1.44) | (25.01) | (13.61) | (7.41) | (15.74) | (5.84) |
| VAR-16 | 19.85 | 12.95 | 18.15 | 12.73 | 11.44 | 12.94 | 14.09 | 19.1 | 16.79 | 16.89 | 14.22 | 16.67 |
| | (8.57) | (8.32) | (6.51) | (8.95) | (12.34) | (6.9) | (12.39) | (19.94) | (9.73) | (7.23) | (6.61) | (6.03) |
| VAR-17 | 46.08 | 33.4 | 42.26 | 39.69 | 20.56 | 33.54 | 58.33 | 34.05 | 51.01 | 32.95 | 27.56 | 31.3 |
| | (15.36) | (21.73) | (13.98) | (17.83) | (26.63) | (15.78) | (28.06) | (31.46) | (21.21) | (13.33) | (19.52) | (11.52) |
| VAR-18 | 39.6 | 46.44 | 48.32 | 46.62 | 26.16 | 41.8 | 38.91 | 34.27 | 41.53 | 33.51 | 32.82 | 37.23 |
| | (13.32) | (13.06) | (15.66) | (20.9) | (14.94) | (20.87) | (23.32) | (18.12) | (17.66) | (13.07) | (13.05) | (13.46) |
| VAR-19 | 30.76 | 29.81 | 29.11 | 37.04 | 12.87 | 29.58 | 31.27 | 25.32 | 28.5 | 26.94 | 33 | 26.93 |
| | (9.05) | (16.31) | (11.62) | (12.49) | (18.38) | (9.62) | (16.79) | (28.59) | (12.29) | (10.27) | (19.02) | (9.73) |
| VAR-20 | 36.21 | 32.25 | 35.04 | 36.64 | 20.06 | 31.33 | 49.62 | 26.48 | 42.7 | 29.3 | 29.69 | 29.4 |
| | (13.62) | (21.73) | (13.76) | (24.05) | (22.02) | (12.32) | (24.28) | (32.17) | (16.64) | (13.06) | (21.79) | (9.08) |
| VAR-21 | 23.47 | 12.36 | 20.36 | 22.13 | 16.85 | 21.53 | 37 | 8.74 | 27.41 | 24.39 | 13.27 | 21.36 |
| | (27.68) | (26.79) | (17.65) | (37.18) | (37.09) | (24.68) | (51.77) | (21.04) | (37.29) | (26.01) | (25.66) | (17.99) |
| VAR-22 | 38.77 | 25.75 | 34.63 | 31.58 | 13.48 | 25.78 | 39.39 | 30.3 | 36.15 | 28.02 | 25.2 | 26.72 |
| | (13.27) | (16.01) | (9.07) | (10.24) | (17.22) | (12.02) | (14.1) | (22.62) | (15.07) | (5.54) | (14.16) | (8.68) |
| VAR-23 | 51.52 | 58.39 | 55.99 | 60.32 | 43.11 | 56.21 | 41.29 | 31.15 | 39.13 | 43.48 | 39.58 | 43.65 |
| | (29.37) | (38.13) | (21.87) | (35.53) | (35.98) | (27.31) | (30.15) | (53.95) | (25.85) | (20.42) | (26.55) | (18.01) |
| VAR-24 | 44.43 | 32 | 42.19 | 31.7 | 27.24 | 32.23 | 41.26 | 35.36 | 41.63 | 32.99 | 34.26 | 35.66 |
| | (10.55) | (9.43) | (10.96) | (11.02) | (17.53) | (9.43) | (19.62) | (12.96) | (8.83) | (12.99) | (1.79) | (4.91) |
| VAR-25 | 41.62 | 36.76 | 42.47 | 34.38 | 44.1 | 41.81 | 50.11 | 28.85 | 44.52 | 30.34 | 35.36 | 34.49 |
| | (11.35) | (16.61) | (8.59) | (15.29) | (9.06) | (3.35) | (19.04) | (17.12) | (10.18) | (10.03) | (9.26) | (5.08) |
| VAR-26 | 65.32 | 53.14 | 62.18 | 49.66 | 62.03 | 54.52 | 70.05 | 83.63 | 75.17 | 51.27 | 44.21 | 49.62 |
| | (17.1) | (28.32) | (19.61) | (27.92) | (35.87) | (23.22) | (32.25) | (54.36) | (33.76) | (20.09) | (24.65) | (16.9) |
| VAR-27 | 38.27 | 22.26 | 34.46 | 32.68 | 17.83 | 28.91 | 32.46 | 29.74 | 34.95 | 39.05 | 26.49 | 37.04 |
| | (15.42) | (11.66) | (11.23) | (20.96) | (11.63) | (11.03) | (24.39) | (23.88) | (20.95) | (16.07) | (12.32) | (12.14) |
| VAR-28 | 38.96 | 29.36 | 40.28 | 38.34 | 25.15 | 37.85 | 43.36 | 28.84 | 42.39 | 24.09 | 28.64 | 31.72 |
| | (17.5) | (14.26) | (15.84) | (17.65) | (15.74) | (15.75) | (28.26) | (15.42) | (19.57) | (13.44) | (8.64) | (9.51) |
| VAR-29 | 16.28 | 11.98 | 15.75 | 25.62 | 26.95 | 29.09 | 24.51 | 25.27 | 27.18 | 9.53 | 11.16 | 11.11 |
| | (8.08) | (9.54) | (7.35) | (12.93) | (15.5) | (12.6) | (16.52) | (18.59) | (13.83) | (5.96) | (9.16) | (5.82) |
| VAR-30 | 63.21 | 49.85 | 63.73 | 44.73 | 39.06 | 47.85 | 52.26 | 46.76 | 55.75 | 35.21 | 38.44 | 40.82 |
| | (18.35) | (20.76) | (16.73) | (23.99) | (18.19) | (20.66) | (24.01) | (23.37) | (22.08) | (13.77) | (18.07) | (12.63) |
| VAR-31 | 45.47 | 40.05 | 44.48 | 44.43 | 33.94 | 41.62 | 43.67 | 48.55 | 46.08 | 35.11 | 30.77 | 34.3 |
| | (14.32) | (16.59) | (9.95) | (13.61) | (19.27) | (9.26) | (20.64) | (27.77) | (15.69) | (11.8) | (14.13) | (11.48) |
| VAR-32 | 37.89 | 26.2 | 34.57 | 41.53 | 20.79 | 34.95 | 36.59 | 40.45 | 38.08 | 21.63 | 32.49 | 25.07 |
| | (18.12) | (28.55) | (14.82) | (26.52) | (28.02) | (18.53) | (30.94) | (44.97) | (25.25) | (14.12) | (23.7) | (10.79) |
| VAR-33 | 47.9 | 26.08 | 41.98 | 52.02 | 28.63 | 45.63 | 60.45 | 50.81 | 61.01 | 46.75 | 36.36 | 45.73 |
| | (11.05) | (11.97) | (11.09) | (1.59) | (10.73) | (7.7) | (14.88) | (2.32) | (9.28) | (8.14) | (10.71) | (5.15) |
| VAR-34 | 29.89 | 24.1 | 28.12 | 29.59 | 26.14 | 28.39 | 32.25 | 24.75 | 29.94 | 25.36 | 23.87 | 24.86 |
| | (11.9) | (14.81) | (9.73) | (12.74) | (17.11) | (7.9) | (14.21) | (22.2) | (13.55) | (9.94) | (14.43) | (7.81) |
| VAR-35 | 23.63 | 18.03 | 22.88 | 31.26 | 9.16 | 23.62 | 25.86 | 11.93 | 21.68 | 15.56 | 17.6 | 17.45 |
| | (11.93) | (13.62) | (8.69) | (19.59) | (11.45) | (12.28) | (20.3) | (21.1) | (13.98) | (9.01) | (11.21) | (7.78) |
| VAR-36 | 34.07 | 18.45 | 29.39 | 20.51 | 3.88 | 15.39 | 20.97 | 14.02 | 18.73 | 21.64 | 14.58 | 19.46 |
| | (13.74) | (19.62) | (12.53) | (17.12) | (8.28) | (10.2) | (15.87) | (27.82) | (13.1) | (10.27) | (16.56) | (9.74) |
| VAR-37 | 31.8 | 32.61 | 32.55 | 25.31 | 19.2 | 23.62 | 29.93 | 39.06 | 33.4 | 25.44 | 23.9 | 25.32 |
| | (8.97) | (14.2) | (5.26) | (14.61) | (11.98) | (10.45) | (14.29) | (19.32) | (12.8) | (5.45) | (12.6) | (8) |
| VAR-38 | 32.61 | 30.62 | 35.58 | 29.35 | 20.33 | 28.5 | 35.93 | 35.05 | 40.06 | 29.19 | 27.11 | 31.56 |
| | (0.75) | (0.87) | (0.61) | (0.55) | (0.62) | (0.49) | (0.67) | (0.96) | (0.69) | (0.58) | (0.83) | (0.53) |
| VAR-39 | 34.13 | 21.37 | 31.46 | 27.54 | 11.48 | 22.38 | 24.63 | 28.4 | 29.38 | 24.79 | 29.65 | 29.81 |
| | (11.48) | (12.46) | (10.24) | (17.24) | (10.3) | (8.15) | (18.16) | (15.48) | (12.02) | (11.28) | (12.54) | (8.84) |
| VAR-40 | 50.91 | 50.56 | 53.98 | 49.27 | 56.93 | 56.46 | 66.07 | 55.73 | 66.05 | 44.58 | 38.96 | 44.99 |
| | (15.38) | (3.16) | (7.41) | (12.73) | (5.25) | (9) | (20.41) | (3.17) | (8.5) | (11.45) | (7.81) | (8.5) |
| VAR-41 | 32.26 | 29.43 | 33.11 | 26.45 | 14.33 | 22.99 | 30.8 | 38.66 | 36.1 | 39.3 | 29.46 | 37.66 |
| | (14.21) | (22.6) | (14.97) | (19.67) | (16.48) | (13.01) | (24.3) | (33.62) | (21.36) | (15.54) | (14.79) | (11.72) |
| VAR-42 | 35.73 | 60.54 | 43.28 | 38.99 | 47.56 | 41.93 | 54.24 | 60.15 | 56.22 | 37.28 | 45.3 | 39.75 |
| | (16.93) | (37.39) | (17.59) | (27.64) | (45.27) | (21.9) | (37.56) | (64.34) | (28.82) | (16.8) | (33.92) | (16.88) |
| VAR-43 | 44.88 | 45.2 | 45.64 | 59.49 | 42.79 | 54.69 | 64.31 | 46.19 | 59.36 | 49.68 | 52.76 | 51.34 |
| | (12.55) | (17.89) | (14.71) | (30.76) | (24.11) | (19.44) | (22.05) | (32.53) | (20.13) | (15.94) | (20.43) | (11.97) |
| VAR-44 | 35.75 | 31.91 | 36.74 | 33.06 | 27.05 | 33.17 | 42 | 39.86 | 44.29 | 33.59 | 25.42 | 32.49 |
| | (7.97) | (8.18) | (8.73) | (8.99) | (1.22) | (0.75) | (12.46) | (10.62) | (2.61) | (6.36) | (6.72) | (5.02) |
| VAR-45 | 50.83 | 28.98 | 44.11 | 29.17 | 4.89 | 21.9 | 68.05 | 22.69 | 55.05 | 38.35 | 32.54 | 35.84 |
| | (25.27) | (35.2) | (22.44) | (29.05) | (11.43) | (16.85) | (46.61) | (51.04) | (29.34) | (19.38) | (36.61) | (17.04) |
| VAR-46 | 33.6 | 33.61 | 34.22 | 29.03 | 23.28 | 27.61 | 34.61 | 34.24 | 35.14 | 27.43 | 29.56 | 28.59 |
| | (11.62) | (16.84) | (11.78) | (15.89) | (17.08) | (9.78) | (17.09) | (28.19) | (14.54) | (10.28) | (15.23) | (11.53) |
| VAR-47 | 42.31 | 34.68 | 42.45 | 34.37 | 48.04 | 44.7 | 56.6 | 41.5 | 54.6 | 44.02 | 37.54 | 44.72 |
| | (22.64) | (26.56) | (20.56) | (28.71) | (35.14) | (25.66) | (51.07) | (42.54) | (33.45) | (22.58) | (22.71) | (17.56) |
| VAR-48 | 31.37 | 22.46 | 28.91 | 35.96 | 17.71 | 30.19 | 40.71 | 29.5 | 37.59 | 38.83 | 20.18 | 33.48 |
| | (13.75) | (19.61) | (14.64) | (26.18) | (24.88) | (17.27) | (27.95) | (35.99) | (23.83) | (19.5) | (17.92) | (13.91) |
| VAR-49 | 21.28 | 15.37 | 19.41 | 30.24 | 16.52 | 25.75 | 31.21 | 18.33 | 27.26 | 17.74 | 13.36 | 16.36 |
| | (11.51) | (21.79) | (12.88) | (21.26) | (30.2) | (18.14) | (26.71) | (38.46) | (19.05) | (12.08) | (21.69) | (9) |
| VAR-50 | 41.14 | 35.32 | 44.23 | 43.99 | 36.17 | 47.33 | 45 | 33.1 | 45.22 | 35.52 | 28.89 | 37.06 |
| | (12.62) | (1.43) | (5.16) | (11.48) | (1.57) | (1.32) | (15.39) | (8.55) | (4.95) | (9.04) | (1.32) | (5.43) |
| VAR-51 | 36.99 | 26.73 | 34.81 | 33.15 | 16.15 | 27.93 | 39.47 | 25.13 | 35.82 | 30.55 | 33 | 32.88 |
| | (6.33) | (10.08) | (2.57) | (8.69) | (12.87) | (8.79) | (11.91) | (12.22) | (10.71) | (0.8) | (8.07) | (5.24) |
| VAR-52 | 22.78 | 21.64 | 21.33 | 25.44 | 9.51 | 20.48 | 23.42 | 27.81 | 22.97 | 15.21 | 21.03 | 15.52 |
| | (9.26) | (20.77) | (8.66) | (13.63) | (19.68) | (8.89) | (15.68) | (32.28) | (13.75) | (7.18) | (17.87) | (8.2) |
| VAR-53 | 43.93 | 43.95 | 50.16 | 42.88 | 19.26 | 35.68 | 49.5 | 53.17 | 58.87 | 26.73 | 28.22 | 31.17 |
| | (21.57) | (21.47) | (20.96) | (27.84) | (21.24) | (23.29) | (42.53) | (27.64) | (29.85) | (16.2) | (16.92) | (13.56) |
| VAR-54 | 15.59 | 10.16 | 14.73 | 33.8 | 9.72 | 24.79 | 20.19 | 4.11 | 13.95 | 9.9 | 4.47 | 8.24 |
| | (13.28) | (12.39) | (9.24) | (24.7) | (13.14) | (13.84) | (26.71) | (9.76) | (15.7) | (8.1) | (8.24) | (5.93) |
| VAR-55 | 39.57 | 30.31 | 37.86 | 31.56 | 19.29 | 28.21 | 44.95 | 42.48 | 46.15 | 27.39 | 28.82 | 29.13 |
| | (16.21) | (17.99) | (14.73) | (14.01) | (20.19) | (10.35) | (22.69) | (33.1) | (21.01) | (11.83) | (14.71) | (10.54) |
| VAR-56 | 18.8 | 24.67 | 21.9 | 17.72 | 22.03 | 20.47 | 22.01 | 18.14 | 21.55 | 21.09 | 18.15 | 20.91 |
| | (8.89) | (14.28) | (8.2) | (11.01) | (17.62) | (7.68) | (16.63) | (15.56) | (13.26) | (7.61) | (11.26) | (6.46) |
| VAR-57 | 18.68 | 18.88 | 20.33 | 14.92 | 24.71 | 21.24 | 20.68 | 29.05 | 26.32 | 14.23 | 24.64 | 20 |
| | (12.16) | (11.77) | (10.14) | (11.63) | (19.76) | (11.66) | (17.25) | (22.82) | (15.57) | (8.57) | (12.98) | (8.67) |
| VAR-58 | 17.7 | 13.77 | 18.73 | 7.47 | 5.66 | 8.01 | 11.95 | 17.54 | 18.58 | 17.5 | 11.65 | 17.05 |
| | (14.2) | (10.3) | (10.3) | (14.28) | (9.59) | (8.85) | (23.73) | (27.93) | (16.51) | (13.17) | (8.79) | (8.84) |
| VAR-59 | 27.86 | 24.31 | 27.27 | 47.98 | 30.52 | 42.86 | 31.37 | 33.13 | 32.67 | 25.63 | 21.86 | 24.91 |
| | (12.79) | (17.03) | (10.27) | (16.95) | (21.29) | (14.68) | (18.98) | (29.85) | (15.61) | (10.79) | (13.7) | (9.27) |
| VAR-60 | 20.59 | 18.11 | 18.83 | 15.51 | 23.4 | 15.77 | 20.04 | 16.54 | 18.08 | 18.46 | 18.22 | 17.28 |
| | (5.67) | (13.11) | (6.12) | (7.07) | (17.18) | (6.14) | (10.69) | (20.33) | (8.08) | (3.95) | (13.57) | (5.7) |
| VAR-61 | 20.79 | 26.11 | 24.18 | 23.1 | 20.26 | 23.48 | 24.51 | 20.46 | 24.34 | 19.55 | 18.57 | 20.26 |
| | (10.44) | (15.35) | (12.26) | (16.55) | (17.84) | (14.93) | (17.1) | (19.33) | (16.48) | (9.9) | (13.01) | (9.85) |
| VAR-62 | 28.09 | 25.85 | 27.46 | 33.15 | 36.35 | 34.23 | 33.98 | 26.28 | 31.71 | 21.02 | 20.78 | 20.98 |
| | (9.48) | (14.91) | (8.76) | (18.02) | (2.28) | (2.69) | (16.28) | (21.07) | (12.96) | (8.52) | (11.43) | (7.65) |

TABLE 23
Fold change in biodistribution of virus particles comprising the indicated variant capsid polypeptide, relative to the biodistribution of virus particles comprising WT AAV9 for non-brain regions,
with the 95% confidence interval width shown in parentheses following each value. CBh indicates measurements from virus particles comprising genomes with the cap gene under the control of the
CBh promoter. hSyn indicates virus genomes with the cap gene under the control of the hSyn promoter. “Aggregated” in relation to a promoter is an average across the CBh and hSyn promoters.

ORGAN

| | | | | | | | | | | | |
|---|---|---|---|---|---|---|---|---|---|---|---|
| Dorsal Root | Dorsal Root | Dorsal Root | | | | | | | | | |
| Ganglion | Ganglion | Ganglion | Liver | Liver | Liver | Spinal Cord | Spinal Cord | Spinal Cord | Spleen | Spleen | Spleen |

PROMOTER

| VAR-# | CBh | hSyn | Aggregated | CBh | hSyn | Aggregated | CBh | hSyn | Aggregated | CBh | hSyn | Aggregated |
|---|---|---|---|---|---|---|---|---|---|---|---|---|
| VAR-1 | 6.91 | 0.94 | 5.64 | 0.24 | 0.24 | 0.22 | 22.19 | 21.22 | 19.91 | 0.54 | 0.58 | 0.51 |
| | (7.35) | (2.35) | (5.74) | (0.14) | (0.13) | (0.1) | (16.7) | (34.15) | (12.65) | (0.47) | (0.88) | (0.41) |
| VAR-2 | 8.94 | 6.57 | 8.63 | 0.25 | 0.24 | 0.26 | 19.85 | 15.59 | 20.02 | 0.5 | 0.7 | 0.65 |
| | (11.06) | (12.07) | (9.3) | (0.12) | (0.15) | (0.1) | (18.63) | (21.7) | (16.45) | (0.41) | (0.66) | (0.43) |
| VAR-3 | 4.27 | 10.28 | 5.5 | 0.31 | 0.24 | 0.28 | 29.52 | 14.04 | 24.4 | 0.36 | 1.09 | 0.52 |
| | (6.64) | (18.19) | (5.16) | (0.16) | (0.1) | (0.12) | (18.59) | (23.63) | (15.19) | (0.35) | (1.04) | (0.41) |
| VAR-4 | 7.65 | 7.21 | 7.38 | 0.25 | 0.2 | 0.23 | 14.24 | 23.28 | 16.18 | 0.95 | 0.96 | 0.93 |
| | (7.78) | (14.72) | (6.98) | (0.11) | (0.12) | (0.11) | (12.61) | (24.84) | (11.55) | (0.52) | (0.72) | (0.39) |
| VAR-5 | 9.26 | 5.3 | 8.19 | 0.21 | 0.19 | 0.21 | 13.51 | 14.54 | 14.23 | 0.66 | 0.69 | 0.69 |
| | (8.91) | (12.25) | (6.35) | (0.11) | (0.1) | (0.1) | (16.03) | (20.54) | (9.87) | (0.41) | (0.57) | (0.4) |
| VAR-6 | 7.55 | 9.49 | 8.3 | 0.21 | 0.2 | 0.21 | 16.18 | 22.72 | 19.02 | 0.51 | 0.42 | 0.49 |
| | (6.37) | (12.93) | (5.49) | (0.12) | (0.11) | (0.09) | (12.3) | (18.81) | (10.81) | (0.4) | (0.42) | (0.38) |
| VAR-7 | 8.88 | 6.63 | 8.18 | 0.31 | 0.3 | 0.3 | 18.76 | 19.72 | 18.44 | 0.59 | 0.66 | 0.59 |
| | (7.59) | (9.81) | (5.2) | (0.12) | (0.14) | (0.12) | (9.1) | (17.99) | (7.2) | (0.38) | (0.55) | (0.37) |
| VAR-8 | 8.05 | 12.87 | 9.82 | 0.16 | 0.12 | 0.15 | 42.24 | 27.44 | 38.05 | 0.31 | 0.1 | 0.25 |
| | (10.12) | (20.07) | (7.75) | (0.1) | (0.07) | (0.05) | (25.41) | (26.94) | (15.68) | (0.3) | (0.16) | (0.29) |
| VAR-9 | 3.51 | 2.32 | 3.21 | 0.35 | 0.35 | 0.36 | 20.31 | 12.79 | 18.14 | 0.77 | 0.56 | 0.72 |
| | (5.9) | (5.51) | (4.72) | (0.17) | (0.13) | (0.18) | (15.17) | (19.06) | (10.59) | (0.46) | (0.48) | (0.4) |
| VAR-10 | 4.72 | 3.6 | 4.58 | 0.26 | 0.22 | 0.26 | 32.3 | 9.43 | 24.2 | 0.44 | 0.72 | 0.6 |
| | (6.94) | (8) | (5.46) | (0.13) | (0.11) | (0.17) | (20.83) | (11.99) | (16.26) | (0.39) | (0.55) | (0.45) |
| VAR-11 | 7.73 | 0.35 | 6.32 | 0.37 | 0.29 | 0.34 | 16.74 | 21.87 | 15.43 | 0.65 | 0.67 | 0.59 |
| | (8.95) | (0.97) | (6.31) | (0.2) | (0.13) | (0.16) | (14.09) | (39.85) | (11.4) | (0.44) | (0.89) | (0.45) |
| VAR-12 | 7.89 | 11.85 | 9.02 | 0.29 | 0.22 | 0.27 | 17.62 | 10.38 | 15.3 | 1.79 | 2.1 | 1.89 |
| | (7.04) | (15.82) | (8.06) | (0.16) | (0.09) | (0.14) | (15.21) | (17.69) | (12.21) | (1.02) | (1.13) | (0.95) |
| VAR-13 | 7.06 | 9.49 | 8.66 | 0.35 | 0.31 | 0.36 | 22.68 | 14.2 | 20.84 | 0.55 | 0.61 | 0.63 |
| | (6.12) | (7.09) | (5.53) | (0.17) | (0.14) | (0.16) | (16.14) | (9.89) | (8.05) | (0.43) | (0.39) | (0.43) |
| VAR-14 | 14.27 | 13.73 | 15.97 | 0.27 | 0.23 | 0.28 | 33.83 | 13.34 | 26.97 | 0.64 | 1.16 | 1.06 |
| | (16.44) | (16.08) | (13.42) | (0.11) | (0.16) | (0.13) | (30.59) | (17.57) | (17.72) | (0.53) | (0.85) | (0.57) |
| VAR-15 | 10.25 | 8.03 | 9.6 | 0.26 | 0.22 | 0.25 | 31.41 | 29.65 | 30.57 | 0.79 | 0.96 | 0.83 |
| | (0.26) | (12.74) | (5.44) | (0.12) | (0.13) | (0.12) | (14.32) | (10.99) | (10.66) | (0.41) | (0.59) | (0.35) |
| VAR-16 | 6.38 | 9.02 | 7.6 | 0.17 | 0.12 | 0.16 | 13.68 | 12.72 | 14.1 | 0.25 | 0.15 | 0.22 |
| | (6.48) | (9) | (5.08) | (0.09) | (0.07) | (0.07) | (10.24) | (10.86) | (10.48) | (0.19) | (0.17) | (0.18) |
| VAR-17 | 4.92 | 1.96 | 4.11 | 0.22 | 0.18 | 0.21 | 25.06 | 20.87 | 23.61 | 0.5 | 0.38 | 0.46 |
| | (6.66) | (4.63) | (5.05) | (0.11) | (0.08) | (0.1) | (17.6) | (24.68) | (15.74) | (0.42) | (0.46) | (0.36) |
| VAR-18 | 9.94 | 8.27 | 10.18 | 0.3 | 0.26 | 0.31 | 32.87 | 27.39 | 34.78 | 0.54 | 0.59 | 0.64 |
| | (9.3) | (9.72) | (8.53) | (0.17) | (0.14) | (0.16) | (23.07) | (13.71) | (15.44) | (0.5) | (0.5) | (0.41) |
| VAR-19 | 8.37 | 9.21 | 8.19 | 0.25 | 0.26 | 0.24 | 31.09 | 19.93 | 26.78 | 0.63 | 0.52 | 0.58 |
| | (8.88) | (15.76) | (5.88) | (0.13) | (0.12) | (0.14) | (14.23) | (25.73) | (11.55) | (0.46) | (0.49) | (0.38) |
| VAR-20 | 6.86 | 8 | 7.17 | 0.32 | 0.34 | 0.32 | 25.31 | 16.05 | 22.31 | 0.77 | 0.45 | 0.67 |
| | (8) | (14.82) | (7.05) | (0.16) | (0.17) | (0.18) | (19.82) | (23.45) | (16.32) | (0.44) | (0.47) | (0.45) |
| VAR-21 | 4.3 | 2.86 | 3.98 | 0.18 | 0.08 | 0.15 | 16.18 | 9.94 | 14.64 | 0.55 | 0.38 | 0.52 |
| | (10.56) | (7.61) | (8.64) | (0.08) | (0.06) | (0.09) | (30.59) | (26.84) | (23.39) | (0.69) | (0.58) | (0.66) |
| VAR-22 | 3.95 | 5.19 | 4.16 | 0.3 | 0.26 | 0.29 | 23.55 | 10.68 | 19.34 | 0.61 | 0.35 | 0.53 |
| | (3.93) | (9.34) | (4.32) | (0.14) | (0.14) | (0.12) | (8.46) | (15.23) | (7.25) | (0.35) | (0.33) | (0.44) |
| VAR-23 | 11.9 | 5.12 | 9.96 | 0.39 | 0.32 | 0.38 | 43.31 | 26.01 | 38.35 | 1.32 | 0.93 | 1.23 |
| | (15.59) | (13) | (9.79) | (0.21) | (0.17) | (0.17) | (37.74) | (34.55) | (25.7) | (1.08) | (0.9) | (0.76) |
| VAR-24 | 7.54 | 4.61 | 6.82 | 0.28 | 0.25 | 0.29 | 23.86 | 26.12 | 26.91 | 0.63 | 0.79 | 0.74 |
| | (8.4) | (6.78) | (4.53) | (0.13) | (0.14) | (0.15) | (9.95) | (12.88) | (8.57) | (0.5) | (0.39) | (0.36) |
| VAR-25 | 5.97 | 4.02 | 5.54 | 0.31 | 0.25 | 0.3 | 31.05 | 23.72 | 30.28 | 0.47 | 0.56 | 0.54 |
| | (6.28) | (7.36) | (4.67) | (0.15) | (0.13) | (0.13) | (12.75) | (1.23) | (9.58) | (0.37) | (0.5 | (0.37) |
| VAR-26 | 16.75 | 15.75 | 16.61 | 0.29 | 0.31 | 0.3 | 36.46 | 27.43 | 33.82 | 0.43 | 0.65 | 0.5 |
| | (17.27) | (26.64) | (12.05) | (0.17) | (0.17) | (0.14) | (28.64) | (32.14) | (22.87) | (0.39) | (0.56) | (0.37) |
| VAR-27 | 8.25 | 1.82 | 5.96 | 0.18 | 0.11 | 0.16 | 28.37 | 12.38 | 23.35 | 0.39 | 0.18 | 0.33 |
| | (8.39) | (4.41) | (6.25) | (0.09) | (0.06) | (0.07) | (21.61) | (14.33) | (17.1) | (0.34) | (0.2) | (0.28) |
| VAR-28 | 9.92 | 6.25 | 9.29 | 0.25 | 0.22 | 0.27 | 27.43 | 16.94 | 26.38 | 0.63 | 0.51 | 0.67 |
| | (10.19) | (8.87) | (7.05) | (0.1) | (0.12) | (0.15) | (21.34) | (9.46) | (20.81) | (0.45) | (0.43) | (0.45) |
| VAR-29 | 4.55 | 3.25 | 4.34 | 0.15 | 0.11 | 0.14 | 17.02 | 8.98 | 14.79 | 0.44 | 0.38 | 0.45 |
| | (5.45) | (7.11) | (4.79) | (0.07) | (0.05) | (0.05) | (15.37) | (11.63) | (10.75) | (0.38) | (0.29) | (0.36) |
| VAR-30 | 7.44 | 7.91 | 8.41 | 0.25 | 0.22 | 0.26 | 45.59 | 21.35 | 38.36 | 0.62 | 0.77 | 0.77 |
| | (8.16) | (12.22) | (6.75) | (0.15) | (0.1) | (0.11) | (23.44) | (20.98) | (17.2) | (0.47) | (0.52) | (0.46) |
| VAR-31 | 5.46 | 5.12 | 5.44 | 0.36 | 0.37 | 0.37 | 21.42 | 29.85 | 24.91 | 0.88 | 0.88 | 0.89 |
| | (6.09) | (10.16) | (4.8) | (0.17) | (0.17) | (0.16) | (14.52) | (21.08) | (10.45) | (0.5) | (0.68) | (0.41) |
| VAR-32 | 3.51 | 6.76 | 4.47 | 0.41 | 0.36 | 0.4 | 9.49 | 16.09 | 11.81 | 0.61 | 1.11 | 0.77 |
| | (7.12) | (15.51) | (8.42) | (0.2) | (0.18) | (0.18) | (15.85) | (29.12) | (15.43) | (0.5) | (0.96) | (0.53) |
| VAR-33 | 9.83 | 6.02 | 8.91 | 0.24 | 0.16 | 0.22 | 37.41 | 16.82 | 30.97 | 0.38 | 0.44 | 0.44 |
| | (5.94) | (7.93) | (5.47) | (0.11) | (0.06) | (0.1) | (15.54) | (14.15) | (10.26) | (0.32) | (0.38) | (0.29) |
| VAR-34 | 10.51 | 12.59 | 11.04 | 0.25 | 0.21 | 0.24 | 28.17 | 15.19 | 23.94 | 1.02 | 1.3 | 1.09 |
| | (6.57) | (13.91) | (6.47) | (0.12) | (0.12) | (0.12) | (10.12) | (15.73) | (9.79) | (0.57) | (1.22) | (0.56) |
| VAR-35 | 4.65 | 1.53 | 3.68 | 0.3 | 0.23 | 0.29 | 12.03 | 9.39 | 11.82 | 0.73 | 0.77 | 0.8 |
| | (6.39) | (2.73) | (4.53) | (0.14) | (0.13) | (0.12) | (11.57) | (15.27) | (8.48) | (0.42) | (0.57) | (0.42) |
| VAR-36 | 3.65 | 5.45 | 4.03 | 0.39 | 0.29 | 0.36 | 15.46 | 12.34 | 14.21 | 0.64 | 0.47 | 0.58 |
| | (6.62) | (13.86) | (6.51) | (0.21) | (0.14) | (0.18) | (13.9) | (20.59) | (10.09) | (0.45) | (0.63) | (0.4) |
| VAR-37 | 7.03 | 9.58 | 7.89 | 0.27 | 0.26 | 0.27 | 17.36 | 21.34 | 19.09 | 0.59 | 0.48 | 0.56 |
| | (4.98) | (9.74) | (5.63) | (0.13) | (0.13) | (0.12) | (14.01) | (18.78) | (7.13) | (0.39) | (0.4) | (0.35) |
| VAR-38 | 7.25 | 10.2 | 9.48 | 0.25 | 0.24 | 0.27 | 23.13 | 26.9 | 28.84 | 0.58 | 0.42 | 0.57 |
| | (0.14) | (0.34) | (0.17) | (0.1) | (0.1) | (0.09) | (0.42) | (0.82) | (0.44) | (0.24) | (0.24) | (0.22) |
| VAR-39 | 6.51 | 14.51 | 10.9 | 0.27 | 0.22 | 0.27 | 26.74 | 25.56 | 29.71 | 0.75 | 0.54 | 0.73 |
| | (6.3) | (0.78) | (7.58) | (0.12) | (0.11) | (0.13) | (21.58) | (12.21) | (11.42) | (0.43) | (0.39) | (0.42) |
| VAR-40 | 9.37 | 8.8 | 9.66 | 0.39 | 0.34 | 0.39 | 38.44 | 23.66 | 34.69 | 0.95 | 0.76 | 0.93 |
| | (9.2) | (8.43) | (6.74) | (0.19) | (0.17) | (0.16) | (8.79) | (14) | (0.87) | (0.49) | (0.47) | (0.49) |
| VAR-41 | 10.64 | 14.6 | 12.74 | 0.16 | 0.11 | 0.15 | 32.35 | 25.38 | 31.67 | 0.34 | 0.44 | 0.41 |
| | (11.12) | (18.25) | (11.19) | (0.08) | (0.07) | (0.06) | (21.94) | (20.53) | (20.6) | (0.32) | (0.46) | (0.41) |
| VAR-42 | 8.16 | 7.69 | 8.05 | 0.36 | 0.42 | 0.37 | 21.18 | 27.67 | 23.4 | 0.75 | 0.67 | 0.73 |
| | (11.73) | (19.47) | (9.29) | (0.17) | (0.19) | (0.18) | (22.53) | (36.28) | (18.68) | (0.49) | (0.8) | (0.52) |
| VAR-43 | 12.48 | 9.33 | 11.68 | 0.41 | 0.46 | 0.43 | 27.76 | 27.89 | 28.3 | 0.82 | 0.79 | 0.82 |
| | (7.79) | (13.82) | (7.05) | (0.17) | (0.19) | (0.18) | (17.88) | (17.78) | (13.35) | (0.49) | (0.75) | (0.51) |
| VAR-44 | 6.85 | 7.47 | 7.54 | 0.25 | 0.24 | 0.26 | 28.07 | 19.72 | 26.63 | 0.4 | 0.4 | 0.43 |
| | (3.79) | (5.11) | (3.18) | (0.11) | (0.12) | (0.11) | (0.74) | (0.9) | (7.76) | (0.27) | (0.34) | (0.26) |
| VAR-45 | 9.48 | 7.37 | 8.76 | 0.33 | 0.21 | 0.29 | 64.8 | 34.01 | 54.35 | 0.89 | 0.48 | 0.77 |
| | (13) | (21.39) | (12.82) | (0.16) | (0.12) | (0.1) | (42.54) | (59.44) | (39.97) | (0.61) | (0.63) | (0.5) |
| VAR-46 | 6.96 | 8.46 | 7.53 | 0.17 | 0.16 | 0.17 | 14.12 | 25.58 | 18.58 | 0.65 | 0.85 | 0.73 |
| | (6.64) | (11.93) | (6.78) | (0.09) | (0.08) | (0.08) | (12.67) | (16.85) | (11.02) | (0.48) | (0.67) | (0.42) |
| VAR-47 | 5.18 | 3.83 | 4.98 | 0.23 | 0.14 | 0.2 | 20.8 | 18.86 | 22.01 | 0.54 | 0.4 | 0.52 |
| | (10.22) | (9.11) | (7.94) | (0.12) | (0.06) | (0.11) | (23.41) | (27.25) | (18.53) | (0.57) | (0.45) | (0.44) |
| VAR-48 | 15.7 | 8.99 | 13.87 | 0.34 | 0.26 | 0.32 | 27.62 | 14.19 | 23.34 | 0.49 | 0.36 | 0.46 |
| | (14.85) | (18.92) | (12.02) | (0.15) | (0.14) | (0.15) | (21.85) | (23.46) | (16.56) | (0.47) | (0.42) | (0.41) |
| VAR-49 | 4.33 | 1.41 | 3.55 | 0.31 | 0.32 | 0.31 | 10.46 | 13.46 | 11.18 | 1.04 | 1.29 | 1.09 |
| | (8.17) | (3.3) | (7.03) | (0.15) | (0.2) | (0.17) | (15.66) | (24.78) | (13.98) | (0.57) | (1.13) | (0.57) |
| VAR-50 | 5.55 | 5.1 | 6.05 | 0.37 | 0.32 | 0.39 | 31.45 | 18.53 | 29.12 | 0.55 | 0.46 | 0.59 |
| | (5.43) | (4.44) | (4.06) | (0.17) | (0.16) | (0.19) | (6.61) | (7.63) | (10.31) | (0.31) | (0.29) | (0.38) |
| VAR-51 | 6.87 | 9.07 | 7.94 | 0.29 | 0.27 | 0.29 | 26.9 | 24.75 | 27.57 | 0.84 | 0.96 | 0.93 |
| | (3.67) | (7.1) | (3.04) | (0.12) | (0.13) | (0.12) | (11.12) | (8.88) | (7.54) | (0.32) | (0.54) | (0.33) |
| VAR-52 | 5.16 | 3.25 | 4.58 | 0.36 | 0.35 | 0.34 | 16.65 | 5.82 | 13.27 | 0.95 | 0.86 | 0.88 |
| | (5.22) | (8.65) | (5.17) | (0.16) | (0.21) | (0.16) | (11.4) | (15.32) | (10.06) | (0.43) | (0.78) | (0.41) |
| VAR-53 | 6.05 | 2.34 | 4.82 | 0.21 | 0.13 | 0.19 | 28.11 | 13.21 | 23.76 | 0.79 | 0.78 | 0.9 |
| | (11.3) | (5.19) | (7.96) | (0.09) | (0.07) | (0.08) | (27.87) | (19.25) | (18.34) | (0.74) | (0.68) | (0.54) |
| VAR-54 | 5.57 | 2.68 | 4.72 | 0.08 | 0.05 | 0.07 | 21.28 | 15.03 | 21.08 | 0.06 | 0.19 | 0.14 |
| | (9.98) | (5.41) | (7.6) | (0.04) | (0.04) | (0.03) | (21.89) | (16.54) | (13.99) | (0.11) | (0.22) | (0.16) |
| VAR-55 | 3.58 | 7.35 | 5.07 | 0.44 | 0.48 | 0.47 | 26.06 | 12.47 | 21.83 | 0.8 | 0.85 | 0.86 |
| | (5.71) | (11.17) | (5.49) | (0.21) | (0.2) | (0.17) | (18.46) | (17.37) | (12.42) | (0.5) | (0.65) | (0.45) |
| VAR-56 | 12.73 | 8.21 | 11.62 | 0.22 | 0.19 | 0.22 | 17.01 | 15.51 | 17.33 | 0.6 | 0.53 | 0.6 |
| | (10.38) | (10.6) | (7.27) | (0.11) | (0.09) | (0.1) | (13.11) | (10.01) | (13.79) | (0.41) | (0.43) | (0.39) |
| VAR-57 | 5.27 | 10.49 | 7.86 | 0.21 | 0.18 | 0.21 | 11.77 | 8.88 | 11.54 | 1.15 | 1.56 | 1.43 |
| | (6.33) | (11.81) | (6.01) | (0.11) | (0.09) | (0.1) | (12.61) | (12.25) | (8.46) | (0.77) | (1.19) | (0.88) |
| VAR-58 | 13.21 | 11.38 | 14.42 | 0.13 | 0.09 | 0.13 | 4.04 | 11.25 | 10.66 | 0.67 | 0.46 | 0.66 |
| | (14.9) | (11.38) | (11.78) | (0.06) | (0.04) | (0.06) | (10.46) | (15.41) | (10.64) | (0.56) | (0.44) | (0.47) |
| VAR-59 | 6.27 | 9.07 | 7.26 | 0.58 | 0.48 | 0.56 | 20.24 | 17.95 | 19.93 | 0.58 | 0.55 | 0.58 |
| | (6.97) | (13.61) | (5.97) | (0.18) | (0.22) | (0.16) | (15.92) | (18.43) | (13.84) | (0.43) | (0.45) | (0.4) |
| VAR-60 | 2.84 | 6.05 | 3.2 | 0.45 | 0.36 | 0.42 | 12.87 | 14.94 | 12.23 | 0.8 | 0.83 | 0.75 |
| | (3.29) | (12.18) | (3.19) | (0.11) | (0.18) | (0.12) | (10.36) | (18.08) | (5.71) | (0.29) | (0.57) | (0.4) |
| VAR-61 | 4.27 | 5.87 | 5.09 | 0.36 | 0.26 | 0.34 | 9.77 | 9.16 | 10.2 | 1.64 | 1.79 | 1.8 |
| | (6.87) | (9.19) | (6) | (0.15) | (0.12) | (0.17) | (13.52) | (15.44) | (9.76) | (0.95) | (1.05) | (1.01) |
| VAR-62 | 5.07 | 6.97 | 5.6 | 0.65 | 0.57 | 0.63 | 15.3 | 9.42 | 13.4 | 0.88 | 0.73 | 0.84 |
| | (4.09) | (10.84) | (4.95) | (0.21) | (0.19) | (0.19) | (9.7) | (12.92) | (11.48) | (0.43) | (0.44) | (0.41) |

TABLE 24
1: Fold change in transduction of virus particles comprising the indicated variant capsid polypeptide, relative to the transduction of virus particles comprising wild-type AAV9
in brain tissues, with the 95% confidence interval width shown in parentheses following each value. CBh indicates measurements from virus particles comprising genomes with the
cap gene under the control of the CBh promoter. hSyn indicates virus particles comprising genomes with the cap gene under the control of the hSyn promoter. “Aggregated”
in relation to a tissue is an average across all measured areas in the specified organ. “Aggregated” in relation to a promoter is an average across the CBh and hSyn promoters.

| | PROPERTY | | | | | | | | | | | |
|---|---|---|---|---|---|---|---|---|---|---|---|---|
| | Trans- | Trans- | Trans- | Trans- | Trans- | Trans- | Trans- | Trans- | Trans- | Trans- | Trans- | Trans- |
| | duction | duction | duction | duction | duction | duction | duction | duction | duction | duction | duction | duction |
| | ORGAN | | | | | | | | | | | |
| | Brain | Brain | Brain | Brain | Brain | Brain | Brain | Brain | Brain | Brain | Brain | Brain |
| | TISSUE | | | | | | | | | | | |
| | | | | Basal | Basal | Basal | Brain | Brain | Brain | | | |
| | Aggregated | Aggregated | Aggregated | Ganglia | Ganglia | Ganglia | stem | stem | stem | Cerebellum | Cerebellum | Cerebellum |
| | PROMOTER | | | | | | | | | | | |
| VAR-# | CBh | hSyn | Aggregated | CBh | hSyn | Aggregated | CBh | hSyn | Aggregated | CBh | hSyn | Aggregated |
| VAR-1 | 156.27 | 174.09 | 142.08 | 281.67 | 297.11 | 261.58 | 107.56 | 139.37 | 99.81 | 149.97 | 132.12 | 142.18 |
| | (32.98) | (65.32) | (33.04) | (124.71) | (349.97) | (115.49) | (49.75) | (78.11) | (32.24) | (80.97) | (178.6) | (73.65) |
| VAR-2 | 199.03 | 167.76 | 209.46 | 405.17 | 310.99 | 400.58 | 132.97 | 89.15 | 127.61 | 211.5 | 178.41 | 209.5 |
| | (55.98) | (39.71) | (43.32) | (238.67) | (179.56) | (156.26) | (57.63) | (46.04) | (44.27) | (130.06) | (152.06) | (137.54) |
| VAR-3 | 149.03 | 121.63 | 135.09 | 254.05 | 264.2 | 248.85 | 128.51 | 52.16 | 99.17 | 120.97 | 215.38 | 136.68 |
| | (37.1) | (45.8) | (32.17) | (130.91) | (224.06) | (120.96) | (57.73) | (43.75) | (30.95) | (84.68) | (226.44) | (75.32) |
| VAR-4 | 171.02 | 137.27 | 154.8 | 266.84 | 247.3 | 254.73 | 97.62 | 81.55 | 88.35 | 213.5 | 137.94 | 196.39 |
| | (35.95) | (47.79) | (21.74) | (101.27) | (184.89) | (111.07) | (40.68) | (43.89) | (30.38) | (72.5) | (140.81) | (74.81) |
| VAR-5 | 180.68 | 134.36 | 167.66 | 380.98 | 246.95 | 343.91 | 109.55 | 75.08 | 98.55 | 159.73 | 140.92 | 156.46 |
| | (41.98) | (41.1) | (26.83) | (166.86) | (176.46) | (130.97) | (50.19) | (39.5) | (31.58) | (91.33) | (135.24) | (84.62) |
| VAR-6 | 134.82 | 122.9 | 134.2 | 214.14 | 216.9 | 219.76 | 97.3 | 79.94 | 93.29 | 134.78 | 129.22 | 134.76 |
| | (26.92) | (27.42) | (22.51) | (95.97) | (115.64) | (82.28) | (30.21) | (34.19) | (24.6) | (76.28) | (102.09) | (68.96) |
| VAR-7 | 136.33 | 125.59 | 128.33 | 229.67 | 232.82 | 224.55 | 110.54 | 71.5 | 93.75 | 111.58 | 133.56 | 114.4 |
| | (22.93) | (33.43) | (19.81) | (90.85) | (117.95) | (82.47) | (25.13) | (34.24) | (18.22) | (56.54) | (94.79) | (52.65) |
| VAR-8 | 164.02 | 111.66 | 149.07 | 273.62 | 277.91 | 283.07 | 115.31 | 51.21 | 92.18 | 128.17 | 101.95 | 122.86 |
| | (47.52) | (42.57) | (36.34) | (157.28) | (233.23) | (134.06) | (48.35) | (29.49) | (39.35) | (89.52) | (117.1) | (70.21) |
| VAR-9 | 144.53 | 117.55 | 139.27 | 282.05 | 276.91 | 288.46 | 98.28 | 65.61 | 88.33 | 126.62 | 112.87 | 124.68 |
| | (41.49) | (34.46) | (28.17) | (119.53) | (168.37) | (120.35) | (44.04) | (34.25) | (32.22) | (73.66) | (110.13) | (85.68) |
| VAR-10 | 147.16 | 118.6 | 149.35 | 256.73 | 225.61 | 264.46 | 126.16 | 69.85 | 110.81 | 109.61 | 147.14 | 127.43 |
| | (37.32) | (34.8) | (29.19) | (134.72) | (147.62) | (112.74) | (64.64) | (37.5) | (39.95) | (81.97) | (106.68) | (76.47) |
| VAR-11 | 116.36 | 110.8 | 99.99 | 180.03 | 165.46 | 160.99 | 72.47 | 53.36 | 58.52 | 134.39 | 134.89 | 128.14 |
| | (38.12) | (75.98) | (27.61) | (129.83) | (261.05) | (106.01) | (41.17) | (71.87) | (26.9) | (97.03) | (237.72) | (82.38) |
| VAR-12 | 125.99 | 134.52 | 129.59 | 265.38 | 212.69 | 250.31 | 95.13 | 89.28 | 93.39 | 105.49 | 115.59 | 108.02 |
| | (39.33) | (44.54) | (25.83) | (121.08) | (162.32) | (102.78) | (45.72) | (40.98) | (31.32) | (66.66) | (109.72) | (70.76) |
| VAR-13 | 138.29 | 124.75 | 148.37 | 185.23 | 183.43 | 201.25 | 104.05 | 59.15 | 92.6 | 126.15 | 200.93 | 158.74 |
| | (28.81) | (20.73) | (24.99) | (90.73) | (110.19) | (89.19) | (33.59) | (21.45) | (25.02) | (60.96) | (49.99) | (58.25) |
| VAR-14 | 151.6 | 134.39 | 174.55 | 301.23 | 250.51 | 320.6 | 92.27 | 69.24 | 99.13 | 122.52 | 139.24 | 140.36 |
| | (56.87) | (39.85) | (41.64) | (211.32) | (178.8) | (178.03) | (61.72) | (36.1) | (36.47) | (125.89) | (117.06) | (116.05) |
| VAR-15 | 173.43 | 119.06 | 153.56 | 300.78 | 175.8 | 263.22 | 113.05 | 70.68 | 96.52 | 178.16 | 175.89 | 176.98 |
| | (21.1) | (31.16) | (18.4) | (91.19) | (129.25) | (90.26) | (29.95) | (29.93) | (18.04) | (68.87) | (107.28) | (54.4) |
| VAR-16 | 125.89 | 98.27 | 122.02 | 198.63 | 152.07 | 190.4 | 87.23 | 51.56 | 76.59 | 118.2 | 84.3 | 110.84 |
| | (23.14) | (17.07) | (20.34) | (86.51) | (97.76) | (76.2) | (29.31) | (25.4) | (20.86) | (65.63) | (71.29) | (52.04) |
| VAR-17 | 149.75 | 100.18 | 132.08 | 277.13 | 231.21 | 262.7 | 97.5 | 50.13 | 79.54 | 140.03 | 110.88 | 133.25 |
| | (37.54) | (36.41) | (30.1) | (133.38) | (211.39) | (118.76) | (40.88) | (36.07) | (28.42) | (80.3) | (108.26) | (72.79) |
| VAR-18 | 189.11 | 123.18 | 181.4 | 380.17 | 224.24 | 345.12 | 138.72 | 69.73 | 120.27 | 155.4 | 148.53 | 162.3 |
| | (44.64) | (28.15) | (28.28) | (171.56) | (124.77) | (141.43) | (51.25) | (29.32) | (34.18) | (84.32) | (97.57) | (80.33) |
| VAR-19 | 143.94 | 107.63 | 125.51 | 257.23 | 146.12 | 221.99 | 97.08 | 53.85 | 78.34 | 182.32 | 190.59 | 179.36 |
| | (36.3) | (38.3) | (23.43) | (132.17) | (172.24) | (96.98) | (35.57) | (31.59) | (25.58) | (66.79) | (167.29) | (74.25) |
| VAR-20 | 129.77 | 110.78 | 123.12 | 268.71 | 163.57 | 237.52 | 85.31 | 63.02 | 76.92 | 147.11 | 96.98 | 135.64 |
| | (34.57) | (41.74) | (27.98) | (125.31) | (175.75) | (108.18) | (42.38) | (41.07) | (29.86) | (87.57) | (113.55) | (75.42) |
| VAR-21 | 128.58 | 115.76 | 134.83 | 309.85 | 76.83 | 231.56 | 61.01 | 77.87 | 77.13 | 88.96 | 93.95 | 93.68 |
| | (68.39) | (64.35) | (66.57) | (284.45) | (159.04) | (226.31) | (67.1) | (73.33) | (58.93) | (119.36) | (208.08) | (145.66) |
| VAR-22 | 127.32 | 98.2 | 114.63 | 251.81 | 163.7 | 223.74 | 96.23 | 60.33 | 81.22 | 97 | 88.97 | 94.36 |
| | (23.36) | (31.96) | (20.06) | (98.13) | (130.1) | (90.83) | (27.72) | (31.31) | (21.99) | (50.58) | (84.96) | (45.16) |
| VAR-23 | 163.35 | 126.14 | 156 | 397.49 | 305.77 | 378.95 | 132.82 | 76.47 | 114.69 | 103.74 | 53.41 | 91.34 |
| | (68.08) | (57.02) | (49.76) | (250.05) | (257.17) | (205.97) | (62.64) | (58.31) | (46.46) | (104.43) | (105.95) | (80.85) |
| VAR-24 | 169.55 | 112.19 | 157.14 | 335.01 | 257.34 | 325.03 | 114.15 | 50.38 | 92.34 | 135.06 | 109.26 | 131.09 |
| | (28.83) | (20.1) | (20.34) | (108.7) | (102.94) | (80.53) | (30.09) | (23.29) | (20.65) | (73.13) | (68.32) | (55.7) |
| VAR-25 | 157.52 | 101.75 | 145 | 260.27 | 187.01 | 247.32 | 116.99 | 53.85 | 95.92 | 167.56 | 119.1 | 157.24 |
| | (28.6) | (25.88) | (21.16) | (120.51) | (107.82) | (91.19) | (35.07) | (25.96) | (19.61) | (63.2) | (66.54) | (56.03) |
| VAR-26 | 199.77 | 117.75 | 172.29 | 383.76 | 237.5 | 341.58 | 105.13 | 60.6 | 89.1 | 196.4 | 163.83 | 189.47 |
| | (54.16) | (44.01) | (46.09) | (227.03) | (236.36) | (168.61) | (53.27) | (45) | (32.7) | (118.04) | (188.62) | (97.44) |
| VAR-27 | 130.16 | 97.73 | 131.85 | 213.64 | 122.9 | 191.78 | 84.13 | 58.03 | 82.92 | 145.67 | 100.19 | 135.78 |
| | (41.93) | (29.44) | (30.98) | (164.44) | (107.8) | (102.18) | (47.96) | (34.56) | (30.26) | (103.28) | (90.97) | (70.74) |
| VAR-28 | 160.75 | 117.76 | 171.99 | 344.81 | 217.79 | 328.6 | 98.57 | 71.59 | 107.46 | 153.99 | 115.16 | 148.8 |
| | (41.72) | (26.68) | (31.66) | (170.72) | (88.39) | (125.47) | (47.49) | (22.31) | (36.85) | (100.79) | (65.68) | (82.26) |
| VAR-29 | 126.76 | 92.95 | 124.41 | 216.63 | 155.85 | 208.06 | 93.06 | 61.03 | 87.76 | 93.29 | 84.7 | 94.36 |
| | (34.51) | (19.97) | (23.27) | (117.24) | (113.49) | (91.27) | (47.25) | (28.44) | (24.84) | (65.88) | (74.11) | (56.17) |
| VAR-30 | 131.2 | 116.36 | 144.01 | 273.58 | 222.08 | 279.49 | 79.39 | 71.24 | 89.23 | 124.02 | 93.87 | 119.07 |
| | (43.88) | (31.83) | (30.58) | (150.75) | (141.59) | (131.76) | (39.41) | (28.61) | (32.42) | (104.87) | (92.95) | (75.08) |
| VAR-31 | 147.8 | 106.12 | 135.3 | 208.77 | 178.27 | 202.39 | 114.6 | 65.7 | 97.43 | 160.74 | 153.41 | 160.25 |
| | (29.52) | (32.05) | (21.98) | (102.81) | (143.9) | (90.77) | (37.16) | (27.72) | (32.08) | (92.26) | (125.82) | (86.89) |
| VAR-32 | 141.29 | 98.26 | 127.09 | 225.68 | 138.1 | 200.17 | 88.87 | 63.62 | 80.02 | 182.59 | 56.29 | 152.97 |
| | (54.66) | (55.15) | (36.39) | (161.84) | (216.56) | (119.89) | (49.09) | (46.72) | (36.93) | (119.88) | (110.1) | (111.64) |
| VAR-33 | 154.99 | 103.45 | 145.15 | 230.59 | 127.68 | 202.98 | 105.94 | 60.23 | 93.6 | 151.02 | 154.04 | 157.89 |
| | (29.19) | (22.28) | (20.67) | (109.3) | (76.76) | (79.51) | (32.78) | (21.44) | (19.62) | (62.43) | (71.73) | (47.96) |
| VAR-34 | 138.97 | 110.91 | 128.75 | 254.83 | 255.86 | 254.24 | 99.37 | 57.25 | 83.37 | 135.56 | 141.54 | 136.67 |
| | (31.3) | (31.25) | (21.9) | (117.46) | (149.05) | (100.6) | (32.78) | (27.46) | (23.33) | (74.66) | (111.94) | (67.43) |
| VAR-35 | 127.21 | 98.8 | 125.39 | 280.52 | 184.16 | 258.93 | 89.85 | 50.9 | 78.83 | 112.75 | 129.66 | 122.22 |
| | (39.63) | (27.92) | (22.39) | (148.91) | (137.52) | (115.94) | (37.08) | (25.03) | (27.46) | (83.37) | (109.67) | (60.25) |
| VAR-36 | 98.7 | 97.15 | 95.53 | 205.67 | 185.02 | 196.27 | 63.53 | 65.09 | 62.04 | 72.94 | 109.7 | 79.6 |
| | (30.15) | (40.92) | (25.06) | (120.97) | (196.53) | (106.17) | (26.72) | (38.71) | (21.96) | (59.01) | (122.59) | (53.75) |
| VAR-37 | 144.41 | 99.18 | 130.24 | 275.04 | 129.5 | 231.91 | 89.86 | 54.89 | 77.67 | 138.63 | 142.52 | 140.64 |
| | (23.07) | (18.94) | (18.91) | (87.95) | (105.82) | (74.91) | (27.13) | (24.45) | (17.72) | (51.93) | (79.34) | (48.04) |
| VAR-38 | 160.51 | 102.33 | 152.17 | 297.73 | 183.2 | 274.06 | 108.14 | 55.22 | 94.15 | 169.19 | 135.39 | 165.58 |
| | (3.21) | (2.98) | (2.25) | (82.97) | (47.31) | (4.58) | (2.17) | (1.72) | (1.55) | (3.41) | (31.87) | (2.93) |
| VAR-39 | 166.95 | 105.41 | 156.35 | 283.91 | 230.5 | 288.66 | 99.38 | 56.98 | 89.97 | 211.93 | 160.41 | 203.26 |
| | (33.32) | (17.22) | (22.13) | (130.43) | (113.21) | (102.57) | (44.31) | (23.43) | (26.92) | (81.84) | (71.87) | (59.52) |
| VAR-40 | 162.23 | 102.46 | 147.46 | 382.78 | 272.75 | 361.46 | 97.11 | 44.13 | 79.1 | 133.36 | 130.16 | 136.49 |
| | (38.51) | (20.29) | (24.43) | (126.77) | (135.42) | (103.49) | (37.15) | (22.14) | (28.05) | (78.4) | (71.97) | (67.06) |
| VAR-41 | 120.08 | 96.8 | 119.35 | 261.53 | 159.93 | 235.87 | 91.55 | 74.81 | 92.07 | 104.66 | 40.12 | 87.13 |
| | (38.5) | (32.51) | (32.19) | (168.56) | (167.17) | (140.81) | (39.91) | (35.6) | (43.23) | (81) | (71.45) | (72.14) |
| VAR-42 | 125.52 | 101.21 | 117.45 | 260.85 | 251.91 | 258.97 | 91.31 | 64.99 | 81.7 | 112.34 | 61.61 | 100.67 |
| | (46.1) | (54.41) | (38.9) | (162.66) | (296.78) | (170.22) | (57.2) | (46.75) | (36.76) | (102.23) | (104.23) | (82.84) |
| VAR-43 | 124.17 | 103.89 | 119.02 | 291.69 | 210.56 | 269.7 | 94.36 | 59.22 | 82.18 | 104.68 | 162.62 | 119.95 |
| | (33.2) | (34.77) | (24.39) | (127.36) | (158.92) | (102.78) | (48.67) | (31.8) | (28.7) | (69.43) | (145.62) | (67.76) |
| VAR-44 | 137.89 | 87.4 | 126.41 | 263.47 | 181.1 | 247.25 | 98.52 | 43.31 | 79.77 | 128.36 | 113.08 | 127.9 |
| | (17.87) | (17.85) | (14.83) | (79.98) | (78.21) | (75.53) | (24.29) | (17.79) | (15.4) | (39.4) | (49.97) | (33.45) |
| VAR-45 | 114.38 | 98.27 | 104.71 | 164.28 | 183.44 | 163.36 | 77.02 | 54.09 | 66.16 | 148.23 | 59.82 | 129.87 |
| | (55.47) | (69.23) | (40.86) | (137.09) | (248.37) | (179.91) | (56.98) | (56.89) | (32.21) | (107.71) | (118.63) | (91.77) |
| VAR-46 | 135.47 | 94.84 | 123.17 | 317.21 | 141.63 | 264.9 | 85.84 | 52.2 | 74.28 | 95.27 | 98.96 | 97.08 |
| | (28.67) | (31.92) | (28.44) | (163.01) | (124.13) | (106.68) | (35.2) | (27.96) | (26.7) | (59.5) | (81.91) | (54.09) |
| VAR-47 | 88.44 | 94.91 | 102.68 | 195.64 | 159.45 | 196.03 | 82.18 | 68.56 | 85.71 | 59.91 | 72.21 | 66.83 |
| | (48.2) | (41.17) | (37.77) | (163.06) | (180.62) | (147.81) | (61.84) | (44.3) | (38.09) | (90.81) | (108.9) | (61) |
| VAR-48 | 129.62 | 92.94 | 118.03 | 315.98 | 158.62 | 269.56 | 95.72 | 48.71 | 78.57 | 102.35 | 129.31 | 109.56 |
| | (39.59) | (43.96) | (30.71) | (176.97) | (194.39) | (158.44) | (46.4) | (39.48) | (31.24) | (88.36) | (143.72) | (83.88) |
| VAR-49 | 90.58 | 88.7 | 88.28 | 130.85 | 115.18 | 124.84 | 54.97 | 46.7 | 50.94 | 85.15 | 112.68 | 90.35 |
| | (39.04) | (50.38) | (30.51) | (109.1) | (200.4) | (121.48) | (37.58) | (46.73) | (28.21) | (85.44) | (178.83) | (62.46) |
| VAR-50 | 148.32 | 88.22 | 139.36 | 239.09 | 199.22 | 253.93 | 101.89 | 40.86 | 81.81 | 161.16 | 102.16 | 146.39 |
| | (28.26) | (4.05) | (15.33) | (108.53) | (9.3) | (64.3) | (32.26) | (12.23) | (15.72) | (65.75) | (33.83) | (43.42) |
| VAR-51 | 120.89 | 94.56 | 117.09 | 263.1 | 194.92 | 249.44 | 79.33 | 57.96 | 75.01 | 113.68 | 115.56 | 116.93 |
| | (21.84) | (13.3) | (13.33) | (80.67) | (69.61) | (71.15) | (26.01) | (16.77) | (13.07) | (42.88) | (33.76) | (33.13) |
| VAR-52 | 93.67 | 88.45 | 85.35 | 160.37 | 176.11 | 154.63 | 66.93 | 53.11 | 57.69 | 85 | 143.57 | 91.48 |
| | (27.4) | (33.3) | (20.29) | (100.56) | (200.42) | (76.11) | (29.36) | (35.12) | (22.96) | (52.25) | (158.95) | (56.72) |
| VAR-53 | 157.89 | 89.37 | 143.86 | 280.11 | 147.65 | 245.21 | 141.2 | 60.7 | 115.86 | 154.41 | 74.07 | 129.96 |
| | (54.09) | (33.01) | (40.66) | (165.81) | (145.14) | (153.59) | (84.32) | (33.76) | (52.94) | (107.32) | (82.88) | (94.89) |
| VAR-54 | 93.86 | 63.77 | 92.53 | 209.02 | 142.21 | 201.06 | 62.72 | 28.08 | 52.24 | 97.16 | 82.81 | 97.62 |
| | (49.46) | (24.98) | (25.68) | (142.54) | (136.36) | (122.77) | (43.64) | (23.86) | (28.57) | (92.54) | (95.15) | (86.29) |
| VAR-55 | 108.99 | 81.98 | 103.92 | 141.07 | 201.95 | 171.17 | 92.59 | 53.96 | 80.73 | 104.63 | 82.31 | 100.38 |
| | (30.01) | (25.69) | (24.1) | (86.63) | (147.71) | (90.37) | (42.95) | (30.55) | (29.68) | (75.22) | (82.13) | (73.89) |
| VAR-56 | 76.13 | 63.97 | 75.57 | 108.62 | 151.41 | 130.08 | 60.32 | 37.2 | 53.57 | 88.76 | 41.14 | 76.64 |
| | (22.89) | (24.02) | (15.78) | (72.37) | (111.66) | (77.56) | (23.93) | (21.84) | (22.06) | (54.74) | (51.45) | (54.1) |
| VAR-57 | 98.22 | 66.86 | 93.04 | 154.51 | 152.96 | 166.94 | 66.52 | 42.28 | 61.56 | 74.52 | 41.57 | 65.97 |
| | (30.07) | (22.77) | (23.85) | (99.81) | (136.37) | (92.59) | (35) | (25.75) | (23.65) | (54.25) | (57.99) | (52.98) |
| VAR-58 | 71.99 | 64.48 | 87.57 | 128.87 | 50.79 | 101.72 | 58.44 | 42.12 | 64.18 | 60.54 | 48.31 | 60.25 |
| | (45.72) | (22.33) | (28.21) | (137.48) | (76.2) | (67.77) | (43.61) | (25.75) | (27.63) | (83.3) | (61.93) | (69.79) |
| VAR-59 | 80.78 | 55.7 | 73.3 | 142.95 | 64.4 | 119.55 | 63.94 | 35.6 | 54.06 | 88.16 | 96.25 | 91.2 |
| | (24.84) | (26.62) | (21.88) | (88.45) | (88.56) | (81.19) | (32.61) | (25.56) | (20.88) | (62.83) | (99.95) | (53.05) |
| VAR-60 | 80.78 | 51.73 | 67.5 | 124.11 | 39.24 | 101.32 | 63.42 | 37.96 | 51.19 | 71.26 | 55.98 | 66.95 |
| | (17.42) | (23.99) | (12.2) | (61.01) | (68.08) | (44.96) | (20.33) | (21.12) | (15.48) | (43.78) | (63.28) | (33.44) |
| VAR-61 | 97.77 | 56.16 | 85.95 | 185.84 | 50.83 | 141.68 | 65.61 | 33.72 | 55.39 | 95.67 | 66.3 | 89.17 |
| | (32.02) | (25.98) | (28.32) | (156.59) | (83.02) | (87.38) | (34.78) | (21.51) | (21.13) | (85.59) | (76.85) | (65.25) |
| VAR-62 | 66.93 | 51.07 | 61.49 | 129.88 | 55.47 | 107.81 | 41.85 | 28.11 | 36.74 | 72.88 | 62.17 | 70.46 |
| | (19.28) | (20.02) | (14.1) | (69.6) | (76.28) | (62.66) | (17.86) | (15.78) | (15.56) | (39.57) | (66.64) | (36.49) |

2: Fold change in transduction of virus particles comprising the indicated variant capsid polypeptide, relative to the transduction of virus particles comprising wild-type AAV9
in brain tissues, with the 95% confidence interval width shown in parentheses following each value. CBh indicates measurements from virus particles comprising genomes with the cap
gene under the control of the CBh promoter. hSyn indicates virus particles comprising genomes with the cap gene under the control of the hSyn promoter. “Aggregated”
in relation to a tissue is an average across all measured areas in the specified organ. “Aggregated” in relation to a promoter is an average across the CBh and hSyn promoters.
“N/D” indicates variant transduction was not detected in the indicated sample.

| | PROPERTY | | | | | | | | | | | |
|---|---|---|---|---|---|---|---|---|---|---|---|---|
| | Transduction | Transduction | Transduction | Transduction | Transduction | Transduction | Transduction | Transduction | Transduction | Transduction | Transduction | Transduction |
| | ORGAN | | | | | | | | | | | |
| | Brain | Brain | Brain | Brain | Brain | Brain | Brain | Brain | Brain | Brain | Brain | Brain |
| | TISSUE | | | | | | | | | | | |
| | | | | Hippo- | Hippo- | Hippo- | | | | Temporal | Temporal | Temporal |
| | Forebrain | Forebrain | Forebrain | campus | campus | campus | Midbrain | Midbrain | Midbrain | Cortex | Cortex | Cortex |
| | PROMOTER | | | | | | | | | | | |
| VAR-# | CBh | hSyn | Aggregated | CBh | hSyn | Aggregated | CBh | hSyn | Aggregated | CBh | hSyn | Aggregated |
| VAR-1 | 238.9 | 189.33 | 190.99 | 91.16 | 405.16 | 133.7 | 93.23 | 219 | 104.28 | 115.76 | 214.86 | 120.12 |
| | (99.37) | (137.65) | (57.32) | (184.57) | (813.6) | (137.47) | (105.43) | (353.29) | (97.58) | (115.75) | (283.72) | (95.13) |
| VAR-2 | 237.39 | 217.98 | 271.89 | 163.91 | N/D | 95.16 | 204.89 | 326.87 | 285.49 | 143.1 | 235.6 | 215.6 |
| | (123.86) | (73.86) | (100.98) | (329.43) | | (192.48) | (191.51) | (218.28) | (190.89) | (131.53) | (145.58) | (151.77) |
| VAR-3 | 190.16 | 158.73 | 168.07 | 110.3 | N/D | 76.31 | 162.94 | 153.01 | 155.68 | 78.78 | 128.67 | 89.43 |
| | (105.51) | (110.11) | (70.8) | (222.72) | | (153.76) | (150.38) | (269.23) | (114.23) | (96.75) | (191.86) | (90.72) |
| VAR-4 | 255.07 | 196.15 | 220.58 | N/D | N/D | N/D | 238.9 | 158.3 | 212.67 | 98.88 | 162.7 | 113.28 |
| | (83.7) | (82.73) | (58.35) | | | | (128.28) | (209.46) | (114.69) | (99.66) | (205.26) | (100.57) |
| VAR-5 | 248.69 | 199.06 | 235.03 | 107.64 | N/D | 69.89 | 244.64 | 125.14 | 209.66 | 136.69 | 168.37 | 152.88 |
| | (121.47) | (81.58) | (71.96) | (217.64) | | (141.1) | (188.28) | (172.39) | (123.62) | (135.7) | (156.06) | (120.82) |
| VAR-6 | 199.68 | 163.73 | 190.93 | N/D | N/D | N/D | 124.94 | 253.66 | 171.07 | 93.08 | 75.84 | 89.1 |
| | (76.9) | (56.61) | (50.26) | | | | (100.46) | (136.05) | (108.12) | (82.22) | (96.15) | (63.8) |
| VAR-7 | 189.76 | 175.28 | 174.99 | 52.41 | 129.34 | 72.14 | 125.07 | 227.23 | 147.17 | 95.67 | 107.79 | 95.81 |
| | (63.4) | (61.6) | (44.55) | (105.34) | (258.66) | (73.13) | (93.75) | (172.92) | (73.68) | (69.58) | (102.1) | (55.18) |
| VAR-8 | 234.24 | 150.97 | 205.8 | 128.17 | 208.43 | 164.64 | 233.03 | 169 | 217.32 | 152.58 | 142.11 | 154.35 |
| | (104.49) | (74.38) | (68.09) | (258.88) | (427.58) | (327.94) | (215.69) | (203.39) | (141.5) | (197.74) | (172.59) | (109.61) |
| VAR-9 | 185.71 | 159.44 | 183.19 | 273.84 | 445.03 | 351.72 | 197.08 | 120.28 | 175.86 | 101.42 | 101.14 | 105.33 |
| | (73.25) | (72.55) | (58.24) | (278.57) | (311.8) | (242.2) | (167.12) | (183.12) | (110.74) | (115.01) | (120.56) | (70.22) |
| VAR-10 | 177.78 | 154.98 | 192.91 | 351.52 | N/D | 209.08 | 213.04 | 109.76 | 183.99 | 65.1 | 153.83 | 119.79 |
| | (101.36) | (63.78) | (67.16) | (362.1) | | (282.65) | (182.2) | (140.96) | (118.38) | (112.12) | (139.42) | (84.26) |
| VAR-11 | 139.97 | 159.47 | 116.47 | N/D | 620.49 | 79.87 | 145.66 | 335.4 | 153.34 | 152.6 | 47.01 | 118.55 |
| | (79.22) | (163.39) | (69.18) | | (1273.33) | (161.06) | (137.84) | (500.76) | (126.14) | (144.68) | (147.06) | (97.73) |
| VAR-12 | 181.68 | 161.51 | 174.11 | 90.84 | N/D | 60.31 | 82.58 | 167.66 | 108.56 | 50.47 | 255.12 | 122.51 |
| | (75.88) | (75.09) | (55.87) | (183.49) | | (121.55) | (113.49) | (222.09) | (107.62) | (73.34) | (173.49) | (99.48) |
| VAR-13 | 211.52 | 176.78 | 226.29 | N/D | 85.98 | 45.26 | 209.12 | 220.76 | 233.53 | 79.11 | 136.79 | 120.22 |
| | (86.88) | (45.95) | (56.48) | | (174.89) | (91.03) | (128.9) | (116.27) | (98.69) | (67.18) | (96.69) | (78.16) |
| VAR-14 | 231.12 | 211.5 | 290.38 | 223.42 | N/D | 117.55 | 177.72 | 115.41 | 169.28 | 124.12 | 168.21 | 183.68 |
| | (183.08) | (79.8) | (120.15) | (447.12) | | (238.02) | (185.1) | (163.69) | (140.88) | (223.73) | (158.25) | (133.17) |
| VAR-15 | 225.43 | 145.63 | 189.73 | 225.43 | N/D | 151.78 | 224.15 | 145.78 | 200.35 | 147.6 | 181.61 | 157.12 |
| | (65.28) | (53.69) | (48.42) | (225.92) | | (114.9) | (111.38) | (146.63) | (84.71) | (75.1) | (162.75) | (71.11) |
| VAR-16 | 192.86 | 157.76 | 192.35 | 64.29 | 91.24 | 80.29 | 153.41 | 123.3 | 149.33 | 81.63 | 138.25 | 112.9 |
| | (61.71) | (29.81) | (42.44) | (129.27) | (184.56) | (82.53) | (98.34) | (124.72) | (89.87) | (78.31) | (103.87) | (68.42) |
| VAR-17 | 180.31 | 123.93 | 155.49 | 197.32 | 408.02 | 264.53 | 201.8 | 55.14 | 158.72 | 164.43 | 170.01 | 165.33 |
| | (87.37) | (79.9) | (58.14) | (395.51) | (427.2) | (267.64) | (158.74) | (140.72) | (105.23) | (119.64) | (200.5) | (108.57) |
| VAR-18 | 221.48 | 157.17 | 228.45 | 114.7 | 105.11 | 127.93 | 234.61 | 227.27 | 260.98 | 172.95 | 135.37 | 179.91 |
| | (108.45) | (68.27) | (70.49) | (231.72) | (214.5) | (191.4) | (203.78) | (189.51) | (140.44) | (151.55) | (105.87) | (108.89) |
| VAR-19 | 160.35 | 133.14 | 137.03 | N/D | N/D | N/D | 130.08 | 266.56 | 151.79 | 124.92 | 116.23 | 114.64 |
| | (76) | (85.35) | (49.01) | | | | (122.57) | (291.15) | (93.24) | (90.44) | (165.44) | (93.28) |
| VAR-20 | 153.42 | 151.13 | 152.35 | N/D | N/D | N/D | 176.96 | 150.71 | 169.15 | 52.97 | 219.67 | 110.12 |
| | (87.89) | (96.93) | (64.09) | | | | (154.76) | (189.91) | (142.65) | (99.31) | (215.88) | (105.75) |
| VAR-21 | 253.58 | 188.49 | 249.08 | N/D | N/D | N/D | 156.69 | 233.61 | 200.86 | 36.48 | 130.96 | 87.18 |
| | (211.34) | (168.62) | (154.48) | | | | (265.24) | (344.46) | (304.43) | (111.78) | (307.63) | (155.9) |
| VAR-22 | 176.08 | 137.06 | 154 | 56.74 | N/D | 38.91 | 128.95 | 129.05 | 126.05 | 85.55 | 134.35 | 97.26 |
| | (61.98) | (61.22) | (44.12) | (114.29) | | (78.03) | (89.87) | (160.65) | (71.44) | (76.71) | (132.25) | (73.45) |
| VAR-23 | 199.62 | 183.71 | 206.42 | N/D | 327.61 | 135.99 | 97.46 | 132.81 | 114.23 | 119.11 | 124.09 | 127.49 |
| | (140.93) | (118.7) | (105.66) | | (669.07) | (275.97) | (150.54) | (188.86) | (146.43) | (187.1) | (182.7) | (127.14) |
| VAR-24 | 253.75 | 163.47 | 233.25 | 126.88 | 160.84 | 154.43 | 180.22 | 206.48 | 203.84 | 120.83 | 109.66 | 126.68 |
| | (72.9) | (36.99) | (51.88) | (254.19) | (165.39) | (154.42) | (99.65) | (112.81) | (78.93) | (84.78) | (73.79) | (71.34) |
| VAR-25 | 198.48 | 133.13 | 186.22 | 140.39 | N/D | 85.13 | 175.48 | 177.69 | 188.99 | 100.28 | 139.46 | 129.69 |
| | (83.52) | (54.93) | (52.1) | (208.76) | | (126.01) | (112.02) | (98.4) | (97.42) | (98.86) | (96.85) | (68.35) |
| VAR-26 | 312.36 | 166.48 | 252.84 | N/D | 274.04 | 97.65 | 253.12 | 111.1 | 210.92 | 176.78 | 103.8 | 152.58 |
| | (124.04) | (107.98) | (96.31) | | (564.78) | (196.87) | (188.15) | (172.79) | (166.8) | (167.57) | (152.43) | (131.64) |
| VAR-27 | 213.09 | 145.36 | 212.6 | N/D | N/D | N/D | 148.55 | 155.69 | 169.82 | 28.29 | 130.93 | 94.77 |
| | (105.8) | (53.01) | (61.86) | | | | (145.39) | (139.87) | (105.83) | (57.41) | (116.5) | (89.57) |
| VAR-28 | 225.75 | 158.84 | 251.45 | 136.39 | N/D | 67.59 | 154.99 | 190.9 | 210.88 | 108.25 | 127.09 | 153.14 |
| | (113.01) | (33.25) | (70.12) | (274.66) | | (136.56) | (154.14) | (135.75) | (124.29) | (118.98) | (86.39) | (106.92) |
| VAR-29 | 196.42 | 138.83 | 193.53 | N/D | 292.22 | 154.82 | 169.3 | 131.63 | 167.21 | 83.46 | 44.28 | 72.57 |
| | (108.24) | (52.9) | (52.45) | | (202.25) | (206.57) | (138.61) | (116.62) | (100.97) | (70.97) | (67.75) | (54.69) |
| VAR-30 | 176.76 | 167.19 | 211.53 | 256.3 | N/D | 145.05 | 160.19 | 100.04 | 147.95 | 91.54 | 158.9 | 147.31 |
| | (113.04) | (52.14) | (75.2) | (294.37) | | (148.72) | (145.56) | (119.57) | (113.49) | (102.33) | (127.57) | (127.4) |
| VAR-31 | 195.13 | 121.49 | 167.14 | 84.46 | 148.56 | 110.12 | 143.96 | 220.84 | 171.78 | 107.25 | 90.04 | 103.24 |
| | (90.77) | (61.16) | (54.17) | (170.49) | (300.29) | (167.77) | (116.6) | (181.9) | (108.12) | (112.17) | (96.12) | (72.62) |
| VAR-32 | 182.59 | 161.33 | 175.84 | N/D | N/D | N/D | 228.24 | 139.97 | 202.57 | 57.97 | 104.62 | 75.36 |
| | (139.47) | (117.85) | (114.31) | | | | (231.75) | (283.23) | (176.64) | (101.47) | (198.08) | (96.44) |
| VAR-33 | 216.72 | 148.56 | 207.22 | 256.53 | 76.61 | 192.34 | 211.34 | 165.63 | 207.73 | 132.34 | 92.86 | 126.22 |
| | (72.53) | (58.95) | (44.11) | (256.33) | (155.5) | (230.07) | (115.11) | (126.25) | (81.61) | (101.49) | (75.06) | (76.58) |
| VAR-34 | 186.32 | 156.92 | 172.82 | 266.83 | N/D | 178.67 | 113.7 | 92.61 | 107.2 | 95.29 | 103.84 | 97.71 |
| | (65.05) | (63.61) | (40.57) | (264.46) | | (136.85) | (84.21) | (112.79) | (71.82) | (89.73) | (106.44) | (76.32) |
| VAR-35 | 161.85 | 149.62 | 176.92 | N/D | N/D | N/D | 136.18 | 84.84 | 123.74 | 71.33 | 104.64 | 94.78 |
| | (85.93) | (55.49) | (53.97) | | | | (126.54) | (99.99) | (96.01) | (85.92) | (105.71) | (66.32) |
| VAR-36 | 107.68 | 153.27 | 120.42 | N/D | 252.31 | 73.56 | 244.72 | 68.19 | 194.21 | 85.46 | N/D | 57.47 |
| | (87.47) | (95.36) | (54.33) | | (520.39) | (148.16) | (185.8) | (140.87) | (143.49) | (92.36) | | (58.7) |
| VAR-37 | 180.08 | 133.89 | 164.04 | 163.19 | 194.25 | 177.8 | 203.99 | 157.5 | 192.02 | 142.47 | 125.08 | 138.9 |
| | (53.58) | (45.65) | (41.92) | (213.75) | (201.31) | (215.82) | (109.7) | (147.81) | (63.87) | (85.39) | (95.06) | (71.95) |
| VAR-38 | 217.78 | 141.62 | 213.7 | 137.3 | 77.05 | 123.26 | 165.38 | 142.3 | 173.8 | 93.71 | 107.01 | 117.97 |
| | (4.48) | (4.01) | (3.69) | (2.69) | (52.26) | (1.84) | (3.32) | (4.2) | (2.92) | (51.78) | (3.17) | (34.84) |
| VAR-39 | 229.45 | 116.25 | 197.99 | 336.91 | 84.33 | 241.03 | 143.57 | 227.92 | 202.46 | 113.64 | 83.05 | 112.98 |
| | (85.88) | (35.26) | (62.29) | (260.45) | (171.19) | (289.72) | (107.84) | (111.29) | (95.88) | (108.74) | (83.5) | (80.5) |
| VAR-40 | 208.55 | 137.56 | 192.61 | 153.11 | 399.15 | 281.38 | 200.09 | 148.33 | 191.34 | 139.75 | 68.04 | 117.24 |
| | (77.79) | (43.9) | (51.07) | (304.4) | (407.15) | (236.15) | (127.16) | (124.44) | (97.25) | (96.77) | (84.08) | (90.32) |
| VAR-41 | 145.93 | 125.03 | 151.87 | 141.07 | N/D | 86.49 | 96.18 | 249.37 | 166.05 | 67.17 | 69.9 | 74.32 |
| | (118.81) | (67.54) | (63.43) | (286.27) | | (174.35) | (112.23) | (223.08) | (125.96) | (105.25) | (108.36) | (73.74) |
| VAR-42 | 113.41 | 123.6 | 118.53 | N/D | 377.87 | 128.47 | 109.92 | 153.19 | 123.33 | 199.61 | 85.88 | 160.59 |
| | (127.57) | (99.41) | (85.66) | | (778.06) | (259.5) | (153.44) | (251.74) | (129.57) | (142.9) | (180.49) | (164.53) |
| VAR-43 | 115.1 | 132.05 | 126.36 | N/D | 166.23 | 60.65 | 126.43 | 112.32 | 123.73 | 125.09 | 75.56 | 108.99 |
| | (71.77) | (67.5) | (53.84) | | (336.29) | (122.5) | (118.8) | (162.52) | (95.75) | (124.64) | (103.47) | (80.07) |
| VAR-44 | 181.32 | 109.38 | 163.22 | 94.74 | 57.8 | 85.69 | 139.96 | 171.83 | 164.53 | 97.75 | 100.71 | 109.35 |
| | (59.42) | (40.09) | (32.43) | (142.5) | (117.44) | (86.49) | (85.17) | (90.98) | (64.45) | (65.83) | (32.95) | (52.75) |
| VAR-45 | 115.24 | 154.3 | 120.85 | N/D | N/D | N/D | 284.83 | 74.37 | 226.23 | 49.73 | 166.77 | 79.31 |
| | (87.64) | (148.51) | (79.91) | | | | (278.8) | (226.04) | (208.83) | (99.01) | (314.79) | (114.34) |
| VAR-46 | 179.72 | 156 | 175.06 | 173.73 | 151.74 | 169.67 | 138.2 | 143.54 | 142.52 | 137.88 | 68.97 | 114.88 |
| | (85.13) | (59.84) | (55.77) | (346) | (307.95) | (242.07) | (105.51) | (173.41) | (98.86) | (106.29) | (103.47) | (100.57) |
| VAR-47 | 88.06 | 130.39 | 131.2 | N/D | 265.75 | 137.76 | 105.53 | 107.74 | 115.72 | 36.85 | 80.53 | 64.58 |
| | (96.74) | (98.46) | (83.2) | | (542.09) | (277.62) | (185.93) | (208.74) | (132.1) | (91.37) | (156.44) | (99.73) |
| VAR-48 | 109.41 | 148.24 | 129.68 | 288.44 | N/D | 189.44 | 213.05 | 178.62 | 204.59 | 91.57 | 20.03 | 66.6 |
| | (90.98) | (88.03) | (68.9) | (571.01) | | (195.55) | (195.73) | (217.5) | (168.64) | (125.52) | (60.9) | (74.33) |
| VAR-49 | 112.44 | 137.25 | 119.07 | 310.56 | N/D | 210.56 | 176.45 | 233.48 | 189.51 | 110.91 | 52.36 | 90.48 |
| | (101.12) | (116.43) | (67.73) | (336.66) | | (217.98) | (197.71) | (299.3) | (167.93) | (132.58) | (107.65) | (82.58) |
| VAR-50 | 193.54 | 113.74 | 186.82 | 123.36 | 240.99 | 228.86 | 175.22 | 130.26 | 176.55 | 117.48 | 116.84 | 143.04 |
| | (68.38) | (27.06) | (42.83) | (185.73) | (9.6) | (135.74) | (110.36) | (72.2) | (73.56) | (89.53) | (36.7) | (61.38) |
| VAR-51 | 148.7 | 107.12 | 139.9 | 92.74 | 66.45 | 87.05 | 131.73 | 98.77 | 125.35 | 92 | 135.92 | 117.88 |
| | (46.59) | (30.86) | (34.78) | (184.71) | (134.22) | (89.38) | (70.43) | (83.76) | (69.39) | (63.6) | (6.65) | (60.6) |
| VAR-52 | 131.25 | 78.56 | 102.46 | 219.59 | 240.15 | 208.65 | 99.81 | 129.81 | 100.15 | 63.9 | 127.35 | 73.35 |
| | (67.01) | (67.33) | (49.63) | (220.98) | (492.55) | (157.58) | (90.94) | (252.1) | (88.4) | (63.9) | (160.93) | (61.22) |
| VAR-53 | 151.3 | 131.36 | 178.16 | 199.44 | 340.73 | 326.52 | 181.31 | 115.11 | 169.79 | 79.14 | 51.63 | 76.53 |
| | (125.49) | (70.14) | (77.45) | (401.54) | (353.59) | (342.61) | (228.25) | (158.3) | (161.71) | (139.63) | (81.38) | (93.33) |
| VAR-54 | 116.08 | 81.88 | 121.12 | N/D | N/D | N/D | 60.4 | 61.77 | 69.76 | 70.31 | 115.43 | 113.55 |
| | (102.59) | (51.8) | (60.64) | | | | (102.09) | (105.17) | (104.28) | (125.52) | (106.79) | (108.82) |
| VAR-55 | 137.1 | 88.47 | 123.03 | 209.26 | N/D | 131.23 | 95.12 | 122.8 | 110.23 | 99.65 | 80.32 | 97.4 |
| | (77.65) | (60.08) | (45.77) | (417.99) | | (198.99) | (120.31) | (144.61) | (101.96) | (98.34) | (103.71) | (82.56) |
| VAR-56 | 92.31 | 68.23 | 87.53 | 148.73 | 108.15 | 140.04 | 59.15 | 146.15 | 95.23 | 53.12 | 98.32 | 76.59 |
| | (54.94) | (40.84) | (37.88) | (221.9) | (218.15) | (144.59) | (67.26) | (130.02) | (79.96) | (65.32) | (104.23) | (55.08) |
| VAR-57 | 140.36 | 92.32 | 132.53 | 220.02 | N/D | 130.97 | 162.51 | 120.58 | 157.16 | 96.04 | 48.28 | 81.85 |
| | (83.49) | (42.7) | (61.86) | (339.13) | | (194.97) | (154.11) | (153.8) | (110.23) | (97.3) | (87.27) | (82.86) |
| VAR-58 | 107.86 | 112.74 | 159.2 | 417.06 | N/D | 201.85 | 47.39 | 102.96 | 96.89 | N/D | 67.34 | 55.19 |
| | (87.44) | (55.71) | (81.75) | (811.7) | | (208.36) | (96.01) | (139.64) | (107.43) | | (88.59) | (80.38) |
| VAR-59 | 87.74 | 67.71 | 81.83 | 94.24 | N/D | 61.1 | 96.38 | 65.27 | 87.99 | 37.4 | 73.18 | 52.51 |
| | (53.59) | (53.81) | (41.67) | (190.18) | | (123.36) | (108.05) | (129.59) | (65.1) | (61.25) | (101.92) | (52.49) |
| VAR-60 | 108.82 | 75.64 | 86.46 | 78.9 | 147.16 | 85.45 | 103. | 99.43 | 95.7 | 56.36 | 33.44 | 46.73 |
| | (37.98) | (52.52) | (27.21) | (90.24) | (296.21) | (86.26) | (70.8) | (140.32) | (57.47) | (53.63) | (57.79) | (35.6) |
| VAR-61 | 145.54 | 99.76 | 135.97 | N/D | N/D | N/D | 103.7 | 41.21 | 84.34 | 45.27 | 57.76 | 54.91 |
| | (97.36) | (60.5) | (61.27) | | | | (144.91) | (101.72) | (92.92) | (72.84) | (80.39) | (61.11) |
| VAR-62 | 81.53 | 72.9 | 78.05 | 210.17 | 104 | 174.84 | 83.59 | 140.54 | 100.71 | 41.7 | 47.27 | 43.71 |
| | (51.53) | (43.81) | (32.23) | (160.57) | (210.06) | (210.24) | (76.59) | (127.55) | (69.37) | (46.47) | (78.81) | (36.02) |

TABLE 25
Fold change in transduction of virus particles comprising the indicated variant capsid polypeptide, relative to the
transduction of virus particles comprising wild-type AAV9 in non-brain tissues, with the 95% confidence interval
width shown in parentheses following each value. CBh indicates measurements from virus particles comprising genomes
with the cap gene under the control of the CBh promoter. hSyn indicates virus particles comprising genomes with
the cap gene under the control of the hSyn promoter. “Aggregated” in relation to a promoter is an average
across the CBh and hSyn promoters. “N/D” indicates variant transduction was not detected in the indicated sample.

| | PROPERTY | | | | | | | | | | | |
|---|---|---|---|---|---|---|---|---|---|---|---|---|
| | Trans- | Trans- | Trans- | Trans- | Trans- | Trans- | Trans- | Trans- | Trans- | Trans- | Trans- | Trans- |
| | duction | duction | duction | duction | duction | duction | duction | duction | duction | duction | duction | duction |
| | ORGAN | | | | | | | | | | | |
| | Dorsal | Dorsal | Dorsal | | | | | | | | | |
| | Root | Root | Root | | | | Spinal | Spinal | Spinal | | | |
| | Ganglion | Ganglion | Ganglion | Liver | Liver | Liver | Cord | Cord | Cord | Spleen | Spleen | Spleen |
| | PROMOTER | | | | | | | | | | | |
| | | | Aggre- | | | Aggre- | | | Aggre- | | | Aggre- |
| VAR-# | CBh | hSyn | gated | CBh | hSyn | gated | CBh | hSyn | gated | CBh | hSyn | gated |
| VAR-1 | N/D | N/D | N/D | 0.15 | 0.69 | 0.2 | 106.51 | 38.96 | 83 | N/D | N/D | N/D |
| | | | | (0.15) | (0.85) | (0.19) | (72.25) | (76.99) | (49.46) | | | |
| VAR-2 | N/D | N/D | N/D | 0.14 | 0.17 | 0.16 | 78.86 | 49.84 | 73.45 | N/D | N/D | N/D |
| | | | | (0.19) | (0.29) | (0.16) | (68.92) | (62.55) | (52.79) | | | |
| VAR-3 | 4.1 | N/D | 2.41 | 0.34 | 0.76 | 0.41 | 113.71 | 72.58 | 97.31 | N/D | N/D | N/D |
| | (12.46) | | (7.21) | (0.27) | (0.7) | (0.27) | (79.14) | (90.04) | (58.31) | | | |
| VAR-4 | N/D | N/D | N/D | 0.16 | 0.48 | 0.22 | 64.22 | 87.61 | 68.48 | N/D | N/D | N/D |
| | | | | (0.16) | (0.47) | (0.18) | (48.09) | (91.11) | (42.97) | | | |
| VAR-5 | N/D | N/D | N/D | 0.17 | 0.36 | 0.22 | 73.98 | 71.23 | 75.05 | N/D | N/D | N/D |
| | | | | (0.17) | (0.46) | (0.17) | (63.9) | (72.96) | (51.6) | | | |
| VAR-6 | 2.72 | N/D | 1.5 | 0.16 | 0.21 | 0.17 | 88.16 | 52.14 | 76.54 | N/D | N/D | N/D |
| | (8.19) | | (4.56) | (0.14) | (0.25) | (0.14) | (53.69) | (54.56) | (38.05) | | | |
| VAR-7 | 5.85 | N/D | 3.42 | 0.41 | 0.66 | 0.45 | 117.07 | 66.33 | 98.04 | N/D | N/D | N/D |
| | (9.8) | | (5.95) | (0.2) | (0.44) | (0.18) | (50) | (60.69) | (35.21) | | | |
| VAR-8 | N/D | N/D | N/D | 0.1 | 0.3 | 0.15 ( | 66.07 | 93.53 | 80.11 | N/D | N/D | N/D |
| | | | | (0.14) | (0.4) | 0.16) | (52.62) | (98.19) | (56.49) | | | |
| VAR-9 | N/D | N/D | N/D | 0.37 | 0.47 | 0.4 | 72.15 | 76.07 | 76.72 | N/D | N/D | N/D |
| | | | | (0.26) | (0.41) | (0.25) | (58.9) | (57.97) | (45.8) | | | |
| VAR-10 | N/D | 3.3 | 2.21 | 0.36 | 0.43 | 0.39 | 88.58 | 69.42 | 88.87 | N/D | N/D | N/D |
| | | (9.99) | (6.64) | (0.28) | (0.36) | (0.23) | (75.8) | (60.66) | (52.85) | | | |
| VAR-11 | N/D | N/D | N/D | 0.28 | 0.6 | 0.3 | 73.42 | 79.55 | 64.32 | N/D | N/D | N/D |
| | | | | (0.2) | (1.01) | (0.25) | (60.22) | (153.13) | (58.18) | | | |
| VAR-12 | N/D | 4.32 | 1.91 | 0.18 | 0.26 | 0.2 | 74.92 | 102.26 | 85 | N/D | N/D | N/D |
| | | (12.98) | (5.77) | (0.17) | (0.31) | (0.15) | (54.8) | (82.84) | (54.66) | | | |
| VAR-13 | 2.85 | N/D | 1.43 | 0.17 | 0.36 | 0.24 | 89.59 | 93.7 | 103.27 | N/D | N/D | N/D |
| | (8.6) | | (4.29) | (0.18) | (0.32) | (0.14) | (56.75) | (39.46) | (37.19) | | | |
| VAR-14 | 8.31 | N/D | 3.72 | 0.42 | 0.46 | 0.45 | 61.42 | 54.74 | 71 | N/D | N/D | N/D |
| | (24.92) | | (10.98) | (0.39) | (0.43) | (0.32) | (87.97) | (64.2) | (59.45) | | | |
| VAR-15 | 4.19 | N/D | 2.4 | 0.2 | 0.32 | 0.23 | 100.71 | 96.04 | 98.05 | 3.13 | N/D | 2.33 |
| | (10.37) | | (6.04) | (0.16) | (0.35) | (0.15) | (45.47) | (56.38) | (41.42) | (9.48) | | (7.01) |
| VAR-16 | N/D | 4.45 | 2.54 | 0.26 | 0.24 | 0.26 | 59.65 | 40.94 | 55.23 | N/D | N/D | N/D |
| | | (9.1) | (6.38) | (0.17) | (0.23) | (0.14) | (42.91) | (41.02) | (30.58) | | | |
| VAR-17 | N/D | N/D | N/D | 0.18 | 0.5 | 0.25 | 81.37 | 26.16 | 62.14 | N/D | N/D | N/D |
| | | | | (0.15) | (0.51) | (0.2) | (52.71) | (52.22) | (43.71) | | | |
| VAR-18 | 4.26 | 5.13 | 6.07 | 0.13 | 0.41 | 0.24 | 98.54 | 60.64 | 92.3 | N/D | N/D | N/D |
| | (12.79) | (12.64) | (12.16) | (0.16) | (0.32) | (0.21) | (69.73) | (52.34) | (53.16) | | | |
| VAR-19 | N/D | N/D | N/D | 0.17 | 0.48 | 0.21 | 100.8 | 70.25 | 86.59 | N/D | N/D | N/D |
| | | | | (0.14) | (0.46) | (0.14) | (48.69) | (72.63) | (42.38) | | | |
| VAR-20 | 4.14 | N/D | 2.35 | 0.28 | 0.54 | 0.33 ) | 103.21 | 64.34 | 89.62 | N/D | N/D | N/D |
| | (12.27) | | (7.13) | (0.23) | (0.56) | (0.25 | (76.12) | (77.89) | (59.19) | | | |
| VAR-21 | N/D | N/D | N/D | 0.29 | 0.42 | 0.33 | 15.79 | 18.47 | 18.72 | N/D | N/D | N/D |
| | | | | (0.45) | (0.87) | (0.41) | (48.14) | (55.78) | (47.03) | | | |
| VAR-22 | N/D | N/D | N/D | 0.28 | 0.5 | 0.31 | 56.54 | 52.47 | 53.53 | N/D | N/D | N/D |
| | | | | (0.17) | (0.45) | (0.17) | (37.15) | (49.74) | (31.37) | | | |
| VAR-23 | N/D | N/D | N/D | 0.32 | 0.4 | 0.34 | 73.68 | 63 | 73.02 | N/D | N/D | N/D |
| | | | | (0.32) | (0.65) | (0.32) | (67.11) | (96.62) | (68.42) | | | |
| VAR-24 | 2.36 | N/D | 1.22 | 0.28 | 0.37 | 0.32 | 80.66 | 61.86 | 79.03 | N/D | 6.28 | 2.37 |
| | (7.06) | | (3.63) | (0.18) | (0.26) | (0.16) | (44.74) | (41.84) | (31.78) | | (18.92) | (7.14) |
| VAR-25 | N/D | N/D | N/D | 0.34 | 0.32 | 0.34 | 101.31 | 75.86 | 98.56 | N/D | N/D | N/D |
| | | | | (0.19) | (0.25) | (0.2) | (54.06) | (36.25) | (32.73) | | | |
| VAR-26 | N/D | N/D | N/D | 0.13 | 0.53 | 0.22 | 86.75 | 52.7 | 75.37 | N/D | N/D | N/D |
| | | | | (0.2) | (0.55) | (0.2) | (83.71) | (78.74) | (52.31) | | | |
| VAR-27 | N/D | N/D | N/D | 0.15 | 0.08 | 0.13 | 57.17 | 36.93 | 54.27 | N/D | N/D | N/D |
| | | | | (0.16) | (0.17) | (0.14) | (52.22) | (40.11) | (45.37) | | | |
| VAR-28 | 10.14 | 2.15 | 6.42 | 0.22 | 0.34 | 0.29 | 89.05 | 45.28 | 79.39 | N/D | N/D | N/D |
| | (25.61) | (6.52) | (12.85) | (0.2) | (0.29) | (0.24) | (75.4) | (45.44) | (52.49) | | | |
| VAR-29 | N/D | 2.38 | 1.63 | 0.17 | 0.36 | 0.24 | 51.2 | 43.71 | 53.69 | N/D | N/D | N/D |
| | | (7.12) | (4.94) | (0.15) | (0.3) | (0.15) | (54.78) | (45.82) | (34.97) | | | |
| VAR-30 | N/D | N/D | N/D | 0.26 | 0.63 | 0.4 | 39.63 | 59.32 | 58.41 | N/D | N/D | N/D |
| | | | | (0.23) | (0.46) | (0.25) | (44.29) | (56.04) | (51.5) | | | |
| VAR-31 | 9.42 | N/D | 5.23 | 0.26 | 0.36 | 0.29 | 58.05 | 61.9 | 60.97 | N/D | N/D | N/D |
| | (18.89) | | (9.08) | (0.22) | (0.4) | (0.18) | (46.27) | (60.51) | (36.18) | | | |
| VAR-32 | N/D | N/D | N/D | 0.32 | 0.84 | 0.43 | 50.2 | 66.39 | 56.65 | N/D | N/D | N/D |
| | | | | (0.3) | (0.88) | (0.33) | (63.77) | (90.27) | (57.01) | | | |
| VAR-33 | N/D | N/D | N/D | 0.19 | 0.22 | 0.21 | 79.34 | 34.37 | 64.54 | N/D | N/D | N/D |
| | | | | (0.13) | (0.23) | (0.13) | (41.13) | (33.32) | (29.49) | | | |
| VAR-34 | N/D | N/D | N/D | 0.17 | 0.4 | 0.21 | 41.26 | 48.33 | 43.47 | N/D | N/D | N/D |
| | | | | (0.14) | (0.33) | (0.14) | (40.49) | (49.57) | (30.29) | | | |
| VAR-35 | N/D | N/D | N/D | 0.32 | 0.37 | 0.34 | 61.77 | 44.27 | 59.03 | N/D | N/D | N/D |
| | | | | (0.24) | (0.35) | (0.2) | (57.32) | (47.27) | (34.36) | | | |
| VAR-36 | 8.01 | N/D | 4.66 | 0.48 | 0.55 | 0.49 | 74 | 64.69 | 69.12 | N/D | N/D | N/D |
| | (20.01) | | (11.62) | (0.27) | (0.59) | (0.29) | (60.41) | (84.22) | (42.43) | | | |
| VAR-37 | 2.02 | N/D | 1.13 | 0.25 | 0.59 | 0.33 | 97.21 | 80.94 | 93.07 | N/D | N/D | N/D |
| | (6.09) | | (3.38) | (0.14) | (0.32) | (0.15) | (43.01) | (49.6) | (34.02) | | | |
| VAR-38 | 1.02 | 3.13 | 2.93 | 0.43 | 0.54 | 0.49 | 82.1 | 62.56 | 84.28 | 1.53 | N/D | 0.95 |
| | (3.07) | (4.35) | (4.43) | (0.14) | (0.17) | (0.14) | (1.37) | (1.92) | (1.41) | (4.62) | | (2.84) |
| VAR-39 | N/D | 2.06 | 1.53 | 0.26 | 0.49 | 0.35 | 83.94 | 102.71 | 108.38 | N/D | N/D | N/D |
| | | (6.25) | (4.58) | (0.19) | (0.35) | (0.17) | (55.72) | (5.65) | (44.44) | | | |
| VAR-40 | 2.85 | N/D | 1.48 | 0.35 | 0.51 | 0.4 | 78.92 | 60.77 | 77.11 | N/D | N/D | N/D |
| | (8.55) | | (4.47) | (0.2) | (0.37) | (0.2) | (55.19) | (50.82) | (38.07) | | | |
| VAR-41 | N/D | N/D | N/D | 0.07 | 0.13 | 0.09 | 33.93 | 35.49 | 37.73 | N/D | N/D | N/D |
| | | | | (0.13) | (0.23) | (0.11) | (52.49) | (49.14) | (40.54) | | | |
| VAR-42 | N/D | 9.22 | 4.07 | 0.31 | 0.46 | 0.34 | 59.83 | 60.56 | 60.35 | N/D | N/D | N/D |
| | | (27.43) | (12.31) | (0.28) | (0.75) | (0.27) | (67.36) | (101.86) | (70.3) | | | |
| VAR-43 | N/D | 4.05 | 1.92 | 0.39 | 0.69 | 0.46 | 70.09 | 37.3 | 59.02 | N/D | N/D | N/D |
| | | (11.99) | (5.74) | (0.22) | (0.47) | (0.24) | (61.12) | (52.15) | (39.75) | | | |
| VAR-44 | 5.28 | 1.41 | 3.62 | 0.35 | 0.34 | 0.36 | 100.93 | 77.8 | 99.69 | N/D | 4.52 | 1.76 |
| | (9.33) | (2.91) | (6.31) | (0.17) | (0.22) | (0.15) | (41.33) | (28.75) | (2.24) | | (13.4) | (5.3) |
| VAR-45 | N/D | N/D | N/D | 0.29 | 0.13 | 0.26 | 43.07 | 52.92 | 43.8 | N/D | N/D | N/D |
| | | | | (0.32) | (0.41) | (0.25) | (74.01) | (113.89) | (49.88) | | | |
| VAR-46 | N/D | 3.7 | 1.79 | 0.17 | 0.3 | 0.2 | 41.79 | 53.5 | 47.45 | N/D | N/D | N/D |
| | | (10.91) | (5.38) | (0.16) | (0.39) | (0.18) | (35.97) | (53.44) | (35.65) | | | |
| VAR-47 | N/D | N/D | N/D | 0.23 | 0.13 | 0.2 | 63.82 | 76.66 | 78.59 | N/D | N/D | N/D |
| | | | | (0.27) | (0.32) | (0.21) | (88.69) | (84.34) | (77.39) | | | |
| VAR-48 | N/D | N/D | N/D | 0.39 | 0.26 | 0.37 | 44.6 | 42.37 | 44.5 | N/D | N/D | N/D |
| | | | | (0.33) | (0.39) | (0.27) | (55.68) | (69.18) | (47.65) | | | |
| VAR-49 | 5.77 | 8.43 | 6.66 | 0.31 | 1.09 | 0.45 | 42.69 | 33.23 | 38.86 | N/D | N/D | N/D |
| | (17.42) | (25.14) | (13.68) | (0.31) | (0.93) | (0.31) | (54.76) | (76.04) | (38.52) | | | |
| VAR-50 | 2.29 | 1.18 | 2.07 | 0.32 | 0.52 | 0.42 | 112.33 | 49.43 | 93.26 | N/D | N/D | N/D |
| | (6.88) | (3.53) | (5.15) | (0.17) | (0.25) | (0.17) | (50.9) | (24.22) | (31.29) | | | |
| VAR-51 | N/D | N/D | N/D | 0.24 | 0.34 | 0.27 | 57.36 | 42.6 | 54.53 | 2.58 | N/D | 1.78 |
| | | | | (0.16) | (0.26) | (0.1) | (34.44) | (30.24) | (24.76) | (7.75) | | (5.38) |
| VAR-52 | N/D | N/D | N/D | 0.39 | 0.64 | 0.42 | 27.67 | N/D | 19.25 | N/D | N/D | N/D |
| | | | | (0.2) | (0.6) | (0.21) | (31.99) | | (21.08) | | | |
| VAR-53 | N/D | N/D | N/D | 0.2 | 0.29 | 0.24 | 20.56 | 27.3 | 29.22 | N/D | N/D | N/D |
| | | | | (0.27) | (0.38) | (0.22) | (42.51) | (53.39) | (43.73) | | | |
| VAR-54 | N/D | N/D | N/D | 0.04 | 0.07 | 0.06 | 36.53 | 29.3 | 39.02 | N/D | N/D | N/D |
| | | | | (0.11) | (0.18) | (0.1) | (62.16) | (44.44) | (43.09) | | | |
| VAR-55 | N/D | 3.69 | 2.08 | 0.4 | 0.59 | 0.46 | 61.12 | 29.13 | 50.64 | N/D | N/D | N/D |
| | | (10.99) | (6.23) | (0.29) | (0.46) | (0.22) | (57.16) | (45.29) | (33.4) | | | |
| VAR-56 | 2.76 | N/D | 1.48 | 0.12 | 0.16 | 0.13 | 30.67 | 10.4 | 23.5 | 4.13 | N/D | 2.87 |
| | (8.29) | | (2.99) | (0.12) | (0.22) | (0.11) | (31.21) | (18.56) | (24.7) | (12.46) | | (8.7) |
| VAR-57 | N/D | 3.11 | 2.07 | 0.23 | 0.19 | 0.22 | 37.8 | 40.85 | 43.95 | N/D | N/D | N/D |
| | | (9.33) | (6.22) | (0.21) | (0.25) | (0.18) | (44.44) | (44.46) | (31.85) | | | |
| VAR-58 | N/D | 6.19 | 6.39 | 0.05 | 0.15 | 0.11 | 28.66 | 36.63 | 44.03 | N/D | N/D | N/D |
| | | (12.73) | (16.06) | (0.1) | (0.22) | (0.13) | (49.68) | (46.34) | (46.91) | | | |
| VAR-59 | N/D | N/D | N/D | 0.38 | 0.63 | 0.44 | 38.86 | 46.44 | 43.06 | N/D | N/D | N/D |
| | | | | (0.26) | (0.44) | (0.24) | (41.55) | (47.2) | (31.64) | | | |
| VAR-60 | N/D | N/D | N/D | 0.33 | 0.86 | 0.39 | 27.11 | 23.58 | 23.89 | N/D | 11.5 | 1.75 |
| | | | | (0.13) | (0.52) | (0.13) | (22.98) | (34.31) | (19.26) | | (23.95) | (5.32) |
| VAR-61 | N/D | N/D | N/D | 0.28 | 0.45 | 0.33 | 47.04 | 19.55 | 37.74 | N/D | N/D | N/D |
| | | | | (0.24) | (0.43) | (0.22) | (46.22) | (32.76) | (31.13) | | | |
| VAR-62 | N/D | N/D | N/D | 0.33 | 0.66 | 0.4 | 25.28 | 20 | 23.47 | N/D | N/D | N/D |
| | | | | (0.2) | (0.4) | (0.16) | (22.92) | (27.12) | (20.01) | | | |

TABLE 26
Profile Scores

| VAR-# | Profile Score |
|---|---|
| VAR-1 | 5.22 |
| VAR-2 | 5.02 |
| VAR-3 | 4.85 |
| VAR-4 | 4.81 |
| VAR-5 | 4.8 |
| VAR-6 | 4.8 |
| VAR-7 | 4.79 |
| VAR-8 | 4.79 |
| VAR-9 | 4.78 |
| VAR-10 | 4.74 |
| VAR-11 | 4.73 |
| VAR-12 | 4.72 |
| VAR-13 | 4.71 |
| VAR-14 | 4.7 |
| VAR-15 | 4.7 |
| VAR-16 | 4.69 |
| VAR-17 | 4.69 |
| VAR-18 | 4.68 |
| VAR-19 | 4.68 |
| VAR-20 | 4.65 |
| VAR-21 | 4.64 |
| VAR-22 | 4.64 |
| VAR-23 | 4.63 |
| VAR-24 | 4.63 |
| VAR-25 | 4.62 |
| VAR-26 | 4.62 |
| VAR-27 | 4.62 |
| VAR-28 | 4.62 |
| VAR-29 | 4.61 |
| VAR-30 | 4.61 |
| VAR-31 | 4.59 |
| VAR-32 | 4.58 |
| VAR-33 | 4.58 |
| VAR-34 | 4.57 |
| VAR-35 | 4.57 |
| VAR-36 | 4.56 |
| VAR-37 | 4.56 |
| VAR-38 | 4.55 |
| VAR-39 | 4.51 |
| VAR-40 | 4.51 |
| VAR-41 | 4.5 |
| VAR-42 | 4.5 |
| VAR-43 | 4.49 |
| VAR-44 | 4.49 |
| VAR-45 | 4.49 |
| VAR-46 | 4.46 |
| VAR-47 | 4.44 |
| VAR-48 | 4.43 |
| VAR-49 | 4.43 |
| VAR-50 | 4.43 |
| VAR-51 | 4.43 |
| VAR-52 | 4.42 |
| VAR-53 | 4.42 |
| VAR-54 | 4.41 |
| VAR-55 | 4.3 |
| VAR-56 | 4.1 |
| VAR-57 | 4.08 |
| VAR-58 | 4.01 |
| VAR-59 | 3.94 |
| VAR-60 | 3.91 |
| VAR-61 | 3.87 |
| VAR-62 | 3.85 |

The sequences of VAR-i to VAR-62 were analyzed and patterns of mutations were identified which were associated with increased brain transduction and/or high profile scores. The mutation sets for each of VAR-i to VAR-62 are set forth in Table 27 below:

TABLE 27
mutation sets of VAR-1 to VAR-62

| VAR-# | Mutation set as compared to SEQ ID NO: 1 |
|---|---|
| VAR-1 | [‘Q579V’, ‘A589T’, ‘T593A’, ‘W595A’, ‘V596L’, ‘N598S’, ‘I601V’] |
| VAR-2 | [‘D551E’, ‘Q579T’, ‘Q592S’, ‘T593V’, ‘W595A’, ‘V596L’, ‘N598S’, ‘I601V’] |
| VAR-3 | [‘Q579V’, ‘Q588S’, ‘T593A’, ‘W595A’, ‘V596L’, ‘N598S’, ‘I601V’] |
| VAR-4 | [‘G549Y’, ‘Q579T’, ‘T593V’, ‘W595A’, ‘V596L’, ‘N598S’, ‘I601V’] |
| VAR-5 | [‘M559S’, ‘Q579T’, ‘H584I’, ‘Q592I’, ‘T593V’, ‘W595A’, ‘V596L’, ‘N598S’] |
| VAR-6 | [‘M559N’, ‘Q579V’, ‘T593S’, ‘W595A’, ‘V596L’, ‘N598S’, ‘I601V’] |
| VAR-7 | [‘Q579V’, ‘Q588G’, ‘T593S’, ‘W595A’, ‘V596L’, ‘N598S’, ‘I601V’] |
| VAR-8 | [‘Y577H’, ‘Q579V’, ‘A581C’, ‘T593V’, ‘W595A’, ‘V596L’, ‘N598S’, ‘I601V’] |
| VAR-9 | [‘I560L’, ‘Q579V’, ‘A589T’, ‘T593A’, ‘W595A’, ‘V596L’, ‘N598S’] |
| VAR-10 | [‘M559A’, ‘Q579V’, ‘H584M’, ‘Q592S’, ‘T593V’, ‘W595A’, ‘V596L’, ‘N598S’, ‘I601V’] |
| VAR-11 | [‘Q579V’, ‘Q588N’, ‘T593A’, ‘W595A’, ‘V596L’,’ N598S’] |
| VAR-12 | [‘M559V’, ‘Q579V’, ‘T593S’, ‘W595A’, ‘V596L’, ‘N598S’, ‘I601V’] |
| VAR-13 | [‘I560Q’, ‘Q579T’, ‘T593V’, ‘W595A’, ‘V596L’, ‘N598S’, ‘I601V’] |
| VAR-14 | [‘Q579T’, ‘Q588N’, ‘A589T’, ‘Q592S’, ‘T593V’, ‘W595A’, ‘V596L’, ‘N598S’, ‘I601V’] |
| VAR-15 | [‘M559Q’, ‘Q579V’, ‘H584Q’, ‘T593V’, ‘W595A’, ‘V596L’, ‘N598S’, ‘I601V’] |
| VAR-16 | [‘Q579V’, ‘Q592I’, ‘T593R’, ‘W595A’, ‘V596L’, ‘N598S’] |
| VAR-17 | [‘M559C’, ‘Q579V’, ‘T593A’, ‘W595A’, ‘V596L’, ‘N598S’] |
| VAR-18 | [‘G549T’, ‘Q579T’, ‘T593V’, ‘W595A’, ‘V596L’, ‘N598S’, ‘I601V’] |
| VAR-19 | [‘Q579V’, ‘T582I’, ‘H584N’, ‘T593V’, ‘W595A’, ‘V596L’, ‘N598S’] |
| VAR-20 | [‘G549A’, ‘Q579V’, ‘T593S’, ‘W595A’, ‘V596L’, ‘N598S’, ‘I601V’] |
| VAR-21 | [‘R550N’, ‘Y577T’, ‘Q579V’, ‘Q592S’, ‘T593V’, ‘W595A’, ‘V596L’, ‘N598S’, ‘I601V’] |
| VAR-22 | [‘R550K’, ‘Q579V’, ‘A589S’, ‘T593A’, ‘W595A’, ‘V596L’, ‘N598S’] |
| VAR-23 | [‘T561S’, ‘Q579V’, ‘H584I’, ‘Q592S’, ‘T593V’, ‘W595A’, ‘V596L’, ‘N598S’, ‘I601V’] |
| VAR-24 | [‘Q579V’, ‘H584l’, ‘Q592S’, ‘T593V’, ‘W595A’, ‘V596L’, ‘N598S’, ‘I601V’] |
| VAR-25 | [‘T561S’, ‘Q579V’, ‘T593A’, ‘W595A’, ‘V596L’, ‘N598S’, ‘I601V’] |
| VAR-26 | [‘M559A’, ‘Q579V’, ‘Q592S’, ‘T593V’, ‘W595A’, ‘V596L’, ‘N598T’, ‘I601V’] |
| VAR-27 | [‘Y577T’, ‘Q579V’, ‘Q592S’, ‘T593V’, ‘W595A’, ‘V596L’, ‘N598S’, ‘I601V’] |
| VAR-28 | [‘V553S’, ‘Q579T’, ‘T593V’, ‘W595A’, ‘V596L’, ‘N598S’, ‘I601V’] |
| VAR-29 | [‘Q579T’, ‘A587H’, ‘Q588N’, ‘A589T’, ‘Q592I’, ‘T593V’, ‘W595A’, ‘V596L’, ‘N598S’, ‘I601V’] |
| VAR-30 | [‘K557N’, ‘Q579V’, ‘H584L’, ‘A589T’, ‘Q592S’, ‘T593V’, ‘W595A’, ‘V596L’, ‘N598S’, ‘I601V’] |
| VAR-31 | [‘Q579T’, ‘H584L’, ‘T593V’, ‘W595A’, ‘V596L’, ‘N598S’] |
| VAR-32 | [‘Q579V’, ‘Q588T’, ‘T593A’, ‘W595A’, ‘V596L’, ‘N598S’] |
| VAR-33 | [‘Y577H’, ‘Q579V’, ‘T582M’, ‘Q592S’, ‘T593V’, ‘W595A’, ‘V596L’, ‘N598S’, ‘I601V’] |
| VAR-34 | [‘G549F’, ‘Q579T’, ‘T593V’, ‘W595A’, ‘V596L’, ‘N598S’, ‘I601V’] |
| VAR-35 | [‘S576W’, ‘Q579V’, ‘A587S’, ‘A589T’, ‘T593V’, ‘W595A’, ‘V596L’, ‘N598S’] |
| VAR-36 | [‘R550K’, ‘Q579V’, ‘A589S’, ‘T593S’, ‘W595A’, ‘V596L’, ‘N598S’] |
| VAR-37 | [‘G549I’, ‘Q579V’, ‘T593S’, ‘W595A’, ‘V596L’, ‘N598S’, ‘I601V’] |
| VAR-38 | [‘G549H’, ‘Q579T’, ‘T593V’, ‘W595A’, ‘V596L’, ‘N598S’, ‘I601V’] |
| VAR-39 | [‘M559Q’, ‘Q579V’, ‘T582I’, ‘T593V’, ‘W595A’, ‘V596L’, ‘N598S’] |
| VAR-40 | [‘Q579V’, ‘T593V’, ‘W595A’, ‘V596L’, ‘N598T’, ‘I601V’] |
| VAR-41 | [‘Y577T’, ‘Q579V’, ‘A589S’, ‘Q592A’, ‘T593V’, ‘W595A’, ‘V596L’, ‘N598S’, ‘I601V’] |
| VAR-42 | [‘Q579T’, ‘H584N’, ‘T593V’, ‘W595A’, ‘V596L’, ‘N598S’] |
| VAR-43 | [‘Q579V’, ‘Q588N’, ‘T593V’, ‘W595A’, ‘V596L’, ‘N598T’, ‘I601V’] |
| VAR-44 | [‘Q579V’, ‘Q588N’, ‘T593A’, ‘W595A’, ‘V596L’, ‘N598S’, ‘I601V’] |
| VAR-45 | [‘Q579T’, ‘Q588N’, ‘A589T’, ‘Q592N’, ‘T593V’, ‘W595A’, ‘V596L’, ‘N598S’, ‘I601V’] |
| VAR-46 | [‘R550L’, ‘Q579T’, ‘T593V’, ‘W595A’, ‘V596L’, ‘N598S’, ‘I601V’] |
| VAR-47 | [‘K557N’, ‘Q579V’, ‘Q588N’, ‘T593A’, ‘W595A’, ‘V596L’, ‘N598S’, ‘I601V’, ‘D609N’] |
| VAR-48 | [‘Q579T’, ‘S586Q’, ‘A587S’, ‘T593V’, ‘W595A’, ‘V596L’, ‘N598S’, ‘I601V’] |
| VAR-49 | [‘Q579T’, ‘Q588N’, ‘A589T’, ‘Q592A’, ‘T593V’, ‘W595A’, ‘V596L’, ‘N598S’] |
| VAR-50 | [‘Q579V’, ‘A587S’, ‘T593S’, ‘W595A’, ‘V596L’, ‘N598S’, ‘I601V’] |
| VAR-51 | [‘Q579T’, ‘Q588N’, ‘A589T’, ‘T593V’, ‘W595A’, ‘V596L’, ‘N598S’, ‘I601V’] |
| VAR-52 | [‘G549N’, ‘Q579T’, ‘A587S’, ‘A589T’, ‘T593V’, ‘W595A’, ‘V596L’, ‘N598S’] |
| VAR-53 | [‘K557L’, ‘M559Q’, ‘Q579V’, ‘T593A’, ‘W595A’, ‘V596L’, ‘N598S’] |
| VAR-54 | [‘S576W’, ‘Q579T’, ‘A581N’, ‘T593V’, ‘W595A’, ‘V596L’, ‘N598S’, ‘I601V’] |
| VAR-55 | [‘Q579V’, ‘H584N’, ‘T593V’, ‘W595A’, ‘V596L’, ‘N598S’, ‘I601V’] |
| VAR-56 | [‘Q579S’, ‘H584L’, ‘T593V’, ‘W595Y’, ‘V596L’, ‘N598S’, ‘I601V’] |
| VAR-57 | [‘M559I’, ‘Q579S’, ‘Q592A’, ‘V596L’, ‘N598S’, ‘I601A’] |
| VAR-58 | [‘Q579S’, ‘A591P’, ‘V596L’, ‘N598S’, ‘I601A’] |
| VAR-59 | [‘E575W’, ‘H584N’, ‘T593V’, ‘W595A’, ‘V596L’, ‘N598S’, ‘I601V’] |
| VAR-60 | [‘M559A’, ‘Q579T’, ‘S586Q’, ‘A587T’, ‘T593V’, ‘W595A’, ‘V596L’, ‘N598S’] |
| VAR-61 | [‘M559T’, ‘Q579V’, ‘A587S’, ‘Q588N’, ‘T593S’, ‘W595A’, ‘V596L’, ‘N598S’] |
| VAR-62 | [‘E575S’, ‘H584M’, ‘T593V’, ‘W595A’, ‘V596L’, ‘N598S’] |

The sequences of VAR-1 to VAR-62 were analyzed and patterns of mutations were identified which were associated with increased brain transduction and/or high profile scores.

One motif which emerged as associated with enhanced properties, for example as described above, was a motif comprising a leucine at a position corresponding to V596 of the VP1 capsid polypeptide of SEQ ID NO:1 and either a serine or threonine at a position corresponding to N598 of the VP1 capsid polypeptide of SEQ ID NO:1. This motif is present in each of VAR-1 to VAR-62. Aggregated brain transduction (in all brain regions, and across both CBH and hSyn promoters) as measured by quantifying cDNA of the uniquely barcoded VP1 capsid polypeptide-encoding transgenes from amongst this set ranged from 61-fold better than wtAAV9 (VAR-62) to over 200-fold better than wtAAV9 (VAR-2). Without being bound by theory, any suitable heterologous transgene included in the virus particles described herein which include one or more capsid proteins comprising this motif is expected to demonstrate the same improvements relative to an otherwise identical virus particle but comprising a fully wild-type (“wt”) AAV9 capsid. In addition, of the more than 5000 additional variants analyzed from this library which had better than wtAAV9 brain transduction and preserved at least 4 of the mutations of the mutation set of VAR-B1, 4817 also included this motif (all 4817 had leucine at a position corresponding to V596 of SEQ ID NO:1 and 4804 and 17 had a serine or threonine, respectively at a position corresponding to N598 of SEQ ID NO:1), with variants within this set having total edit distance from the wtAAV9 VP1 capsid polypeptide ranging from 4 to 15.

Another exemplary motif which emerged as associated with enhanced properties, for example as described above, was a motif comprising a leucine at a position corresponding to V596 of the VP1 capsid polypeptide of SEQ ID NO:1 and either a serine or threonine at a position corresponding to N598 of the VP1 capsid polypeptide of SEQ ID NO:1, wherein the capsid does not include a valine at a position corresponding to T593 of the VP1 capsid polypeptide of SEQ ID NO:1. This motif is present in 20 of the 62 variants described in Table 1. In addition, of the more than 5000 additional variants analyzed from this library which had better than wtAAV9 aggregated (in all brain regions, and across both CBH and hSyn promoters) brain transduction and preserved at least 4 of the mutations of the mutation set of VAR-B1, 777 unique capsid variants also included this motif, with total edit distance from the wtAAV9 VP1 capsid polypeptide ranging from 4 to 13, and with aggregated transduction (in all brain regions, and across both CBH and hSyn promoters) as measured by quantifying cDNA of the uniquely barcoded VP1 capsid polypeptide-encoding transgenes from amongst this set ranging from better than wtAAV9 to 144-fold the level of wtAAV9 and having an average fold-improvement over wtAAV9 in aggregated (in all brain regions, and across both CBH and hSyn promoters) brain transduction of 11.03-fold.

Another motif which emerged as associated with enhanced properties, for example as described above, was a motif comprising a leucine at a position corresponding to V596 of the VP1 capsid polypeptide of SEQ ID NO:1 and either a serine or threonine at a position corresponding to N598 of the VP1 capsid polypeptide of SEQ ID NO:1, and alanine, arginine, serine or threonine at a position corresponding to T593 of the VP1 capsid polypeptide of SEQ ID NO:1. In the 62 variants described herein in Table 1, alanine appeared at the position corresponding to T593 of SEQ ID NO:1 in 10 variants; arginine appeared at the position corresponding to T593 of SEQ ID NO:1 in 1 variant; serine appeared at the position corresponding to T593 of SEQ ID NO:1 in 8 variants; and threonine appeared at the position corresponding to T593 of SEQ ID NO:1 in 2 variants. In addition, of the more than 5000 additional variants analyzed from this library which had better than wtAAV9 aggregated (in all brain regions, and across both CBH and hSyn promoters) brain transduction and preserved at least 4 of the mutations of the mutation set of VAR-B1, 753 also exhibited this pattern of mutations (55 with S, 112 with A, 584 with T and 2 with R), with total edit distance from the wtAAV9 VP1 capsid polypeptide ranging from 4 to 13, and with aggregated transduction (in all brain regions, and across both CBH and hSyn promoters) as measured by quantifying cDNA of the uniquely barcoded VP1 capsid polypeptide-encoding transgenes from amongst this set ranging from better than wtAAV9 to 144-fold the level of wtAAV9 and an average fold-improvement over wtAAV9 in aggregated brain transduction of 11.17-fold.

Another motif which emerged as associated with enhanced properties, for example as described above, was a motif comprising an alanine, tryptophan, or tyrosine at a position corresponding to W595 of the VP1 capsid polypeptide of SEQ ID NO:1, a leucine at a position corresponding to V596 of the VP1 capsid polypeptide of SEQ ID NO:1, and either a serine or threonine at a position corresponding to N598 of the VP1 capsid polypeptide of SEQ ID NO:1. In the 62 variants described herein in Table 1, alanine appeared at the position corresponding to W595 of SEQ ID NO:1 in 59 variants; tryptophan appeared at the position corresponding to W595 of SEQ ID NO:1 in 2 variants (VAR-57 and VAR-58); and tyrosine appeared at the position corresponding to W595 of SEQ ID NO:1 in 1 variant (VAR-56). In addition, of the more than 5000 additional variants analyzed from this library which had better than wtAAV9 aggregated (in all brain regions, and across both CBH and hSyn promoters) brain transduction and preserved at least 4 of the mutations of the mutation set of VAR-B1, 4455 unique variants also comprised this motif (4427 variants with alanine, 27 variants with tryptophan, and 1 variant with tyrosine at the position corresponding to W595 of the VP1 capsid polypeptide of SEQ ID NO:1), with total edit distance from the wtAAV9 VP1 capsid polypeptide ranging from 4 to 15, and with aggregated transduction (in all brain regions, and across both CBH and hSyn promoters) as measured by quantifying cDNA of the uniquely barcoded VP1 capsid polypeptide-encoding transgenes from amongst this set ranging from better than wtAAV9 to 209-fold the level of wtAAV9 and an average fold-improvement over wtAAV9 in aggregated brain transduction of 12.46-fold.

Another motif which emerged as associated with enhanced properties, for example as described above, was a motif comprising an alanine, arginine, serine, threonine or valine at a position corresponding to T593 of the VP1 capsid polypeptide of SEQ ID NO:1, an alanine, tryptophan, or tyrosine at a position corresponding to W595 of the VP1 capsid polypeptide of SEQ ID NO:1, a leucine at a position corresponding to V596 of the VP1 capsid polypeptide of SEQ ID NO:1, and either a serine or threonine at a position corresponding to N598 of the VP1 capsid polypeptide of SEQ ID NO:1. In the 62 variants described herein in Table 1, 60 variants include this motif. In addition, of the more than 5000 additional variants analyzed from this library which had better than wtAAV9 aggregated (in all brain regions, and across both CBH and hSyn promoters) brain transduction and preserved at least 4 of the mutations of the mutation set of VAR-B1, 112 variants also included the motif comprising an alanine at a position corresponding to T593 of the VP1 capsid polypeptide of SEQ ID NO:1, an alanine at a position corresponding to W595 of the VP1 capsid polypeptide of SEQ ID NO:1, a leucine at a position corresponding to V596 of the VP1 capsid polypeptide of SEQ ID NO:1 and a serine at a position corresponding to N598 of the VP1 capsid polypeptide of SEQ ID NO:1, with total edit distance from the wtAAV9 VP1 capsid polypeptide ranging from 6 to 13, and with aggregated transduction (in all brain regions, and across both CBH and hSyn promoters) as measured by quantifying cDNA of the uniquely barcoded VP1 capsid polypeptide-encoding transgenes from amongst this set ranging from better than wtAAV9 to 145-fold the level of wtAAV9 and an average fold-improvement over wtAAV9 in aggregated brain transduction of 28.5-fold; 2 variants included the motif comprising an arginine at a position corresponding to T593 of the VP1 capsid polypeptide of SEQ ID NO:1, an alanine at a position corresponding to W595 of the VP1 capsid polypeptide of SEQ ID NO:1, a leucine at a position corresponding to V596 of the VP1 capsid polypeptide of SEQ ID NO:1 and a serine at a position corresponding to N598 of the VP1 capsid polypeptide of SEQ ID NO:1, with total edit distance from wtAAV9 ranging from 6 to 7, and with aggregated transduction (all brain regions, and across both CBH and hSyn promoters) as measured by quantifying cDNA of the uniquely barcoded VP1 capsid polypeptide-encoding transgenes from amongst this set ranging from better than wtAAV9 to 122-fold the level of wtAAV9 and an average fold-improvement over wtAAV9 in aggregated brain transduction of 61.7-fold; 54 variants included the motif comprising a serine at a position corresponding to T593 of the VP1 capsid polypeptide of SEQ ID NO:1, an alanine at a position corresponding to W595 of the VP1 capsid polypeptide of SEQ ID NO:1, a leucine at a position corresponding to V596 of the VP1 capsid polypeptide of SEQ ID NO:1 and a serine at a position corresponding to N598 of the VP1 capsid polypeptide of SEQ ID NO:1, with total edit distance from wtAAV9 ranging from 6 to 9, and with aggregated transduction (in all brain regions, and across both CBH and hSyn promoters) as measured by quantifying cDNA of the uniquely barcoded VP1 capsid polypeptide-encoding transgenes from amongst this set ranging from better than wtAAV9 to 139-fold the level of wtAAV9 and an average fold-improvement over wtAAV9 in aggregated brain transduction of 42.9-fold; 547 variants included the motif comprising a threonine at a position corresponding to T593 of the VP1 capsid polypeptide of SEQ ID NO:1, an alanine at a position corresponding to W595 of the VP1 capsid polypeptide of SEQ ID NO:1, a leucine at a position corresponding to V596 of the VP1 capsid polypeptide of SEQ ID NO:1 and a serine at a position corresponding to N598 of the VP1 capsid polypeptide of SEQ ID NO:1, with total edit distance from wtAAV9 ranging from 4 to 9, and with aggregated transduction (in all brain regions, and across both CBH and hSyn promoters) as measured by quantifying cDNA of the uniquely barcoded VP1 capsid polypeptide-encoding transgenes from amongst this set ranging from better than wtAAV9 to 71-fold the level of wtAAV9 and an average fold-improvement over wtAAV9 in aggregated brain transduction of 4.4-fold; 3685 variants included the motif comprising a valine at a position corresponding to T593 of the VP1 capsid polypeptide of SEQ ID NO:1, an alanine at a position corresponding to W595 of the VP1 capsid polypeptide of SEQ ID NO:1, a leucine at a position corresponding to V596 of the VP1 capsid polypeptide of SEQ ID NO:1 and a serine at a position corresponding to N598 of the VP1 capsid polypeptide of SEQ ID NO:1, with total edit distance from wtAAV9 ranging from 4 to 15, and with aggregated transduction (in all brain regions, and across both CBH and hSyn promoters) as measured by quantifying cDNA of the uniquely barcoded VP1 capsid polypeptide-encoding transgenes from amongst this set ranging from better than wtAAV9 to 209-fold the level of wtAAV9 and an average fold-improvement over wtAAV9 in aggregated brain transduction of 12.5-fold; 12 variants included the motif comprising a valine at a position corresponding to T593 of the VP1 capsid polypeptide of SEQ ID NO:1, an alanine at a position corresponding to W595 of the VP1 capsid polypeptide of SEQ ID NO:1, a leucine at a position corresponding to V596 of the VP1 capsid polypeptide of SEQ ID NO:1 and a threonine at a position corresponding to N598 of the VP1 capsid polypeptide of SEQ ID NO:1, with total edit distance from wtAAV9 ranging from 6 to 10, and with aggregated transduction (in all brain regions, and across both CBH and hSyn promoters) as measured by quantifying cDNA of the uniquely barcoded VP1 capsid polypeptide-encoding transgenes from amongst this set ranging from better than wtAAV9 to 172-fold the level of wtAAV9 and an average fold-improvement over wtAAV9 in aggregated brain transduction of 57.3-fold.

Another motif which emerged as associated with enhanced properties, for example as described above, was a motif comprising an alanine at a position corresponding to W595 of the VP1 capsid polypeptide of SEQ ID NO:1, a leucine at a position corresponding to V596 of the VP1 capsid polypeptide of SEQ ID NO:1, and either a serine or threonine at a position corresponding to N598 of the VP1 capsid polypeptide of SEQ ID NO:1, wherein the capsid polypeptide further includes an amino acid other than alanine at a position corresponding to 1601 of the VP1 capsid polypeptide of SEQ ID NO:1. In the 62 variants described herein in Table 1, valine appeared at the position corresponding to 1601 of the VP1 capsid polypeptide of SEQ ID NO:1 in 41 variants and isoleucine appeared in 19 variants. In addition, of the more than 5000 additional variants analyzed from this library which had better than wtAAV9 brain transduction and preserved at least 4 of the mutations of the mutation set of VAR-B1, 4368 variants also included this motif, with total edit distance from wtAAV9 ranging from 4 to 15, and with aggregated transduction (in all brain regions, and across both CBH and hSyn promoters) as measured by quantifying cDNA of the uniquely barcoded VP1 capsid polypeptide-encoding transgenes from amongst this set ranging from better than wtAAV9 to 209-fold the level of wtAAV9 and an average fold-improvement over wtAAV9 in aggregated brain transduction of 12.28-fold. Of these, 949 variants comprised an alanine at a position corresponding to W595 of the VP1 capsid polypeptide of SEQ ID NO:1, a leucine at a position corresponding to V596 of the VP1 capsid polypeptide of SEQ ID NO:1, a serine at a position corresponding to N598 of the VP1 capsid polypeptide of SEQ ID NO:1 and a valine at a position corresponding to 1601 of the VP1 capsid polypeptide of SEQ ID NO:1, with total edit distance from wtAAV9 ranging from 6 to 15, and with aggregated transduction (in all brain regions, and across both CBH and hSyn promoters) as measured by quantifying cDNA of the uniquely barcoded VP1 capsid polypeptide-encoding transgenes from amongst this set ranging from better than wtAAV9 to 209-fold the level of wtAAV9 and an average fold-improvement over wtAAV9 in aggregated brain transduction of 27.47-fold; 3412 variants included an alanine at a position corresponding to W595 of the VP1 capsid polypeptide of SEQ ID NO:1, a leucine at a position corresponding to V596 of the VP1 capsid polypeptide of SEQ ID NO:1, a serine at a position corresponding to N598 of the VP1 capsid polypeptide of SEQ ID NO:1 and an isoleucine at a position corresponding to 1601 of the VP1 capsid polypeptide of SEQ ID NO:1, with total edit distance from wtAAV9 ranging from 4 to 15, and with aggregated transduction (in all brain regions, and across both CBH and hSyn promoters) as measured by quantifying cDNA of the uniquely barcoded VP1 capsid polypeptide-encoding transgenes from amongst this set ranging from better than wtAAV9 to 167.7-fold the level of wtAAV9 and an average fold-improvement over wtAAV9 in aggregated brain transduction of 8.1-fold; 5 variants included an alanine at a position corresponding to W595 of the VP1 capsid polypeptide of SEQ ID NO:1, a leucine at a position corresponding to V596 of the VP1 capsid polypeptide of SEQ ID NO:1, a threonine at a position corresponding to N598 of the VP1 capsid polypeptide of SEQ ID NO:1 and a valine at a position corresponding to 1601 of the VP1 capsid polypeptide of SEQ ID NO:1, with total edit distance from wtAAV9 ranging from 6 to 8, and with aggregated transduction (in all brain regions, and across both CBH and hSyn promoters) as measured by quantifying cDNA of the uniquely barcoded VP1 capsid polypeptide-encoding transgenes from amongst this set ranging from 3.4-fold better than wtAAV9 to 172.3-fold the level of wtAAV9 and an average fold-improvement over wtAAV9 in aggregated brain transduction of 113.9-fold.

Another motif which emerged as associated with enhanced properties, for example as described above, is a motif comprising a valine at a position corresponding to 1601 of the VP1 capsid polypeptide of SEQ ID NO:1. In the 62 variants described herein in Table 1, 40 variants include this mutation. In addition, of the more than 5000 additional variants analyzed from this library which had better than wtAAV9 brain transduction and preserved at least 4 of the mutations of the mutation set of VAR-B1, 1127 also included the mutation, with total edit distance from wtAAV9 ranging from 6 to 15, and with aggregated transduction (in all brain regions, and across both CBH and hSyn promoters) as measured by quantifying cDNA of the uniquely barcoded VP1 capsid polypeptide-encoding transgenes from amongst this set ranging from better than wtAAV9 to 209-fold the level of wtAAV9 and an average fold-improvement over wtAAV9 in aggregated brain transduction of 23.6-fold.

Another motif which emerged as associated with enhanced properties, for example as described above, is a motif comprising a threonine at a position corresponding to Q579 of the VP1 capsid polypeptide of SEQ ID NO: 1. In the 62 variants described herein in Table 1, 20 variants include this mutation. In addition, of the more than 5000 additional variants analyzed from this library which have better than wtAAV9 brain transduction and preserved at least 4 of the mutations of the mutation set of VAR-B1, 97 also included the mutation, with total edit distance from wtAAV9 ranging from 7 to 11, and with aggregated transduction (in all brain regions, and across both CBH and hSyn promoters) as measured by quantifying cDNA of the uniquely barcoded VP1 capsid polypeptide-encoding transgenes from amongst this set ranging from better than wtAAV9 to 209-fold the level of wt AAV9 and an average fold-improvement over wtAAV9 in aggregated brain transduction of 73-fold.

Without being bound by theory, any of the motifs described herein, e.g., above, may be combined in a capsid polypeptide with one or more, e.g., 1, 2, 3, 4, 5, 6, 7, 8, 9, 10, 11, 12, 13, 14, 15, or more (e.g., 20, 30, 35, 36, 37, 38 or more) additional mutations relative to a reference capsid polypeptide. Of the more than 7000 variants exhibiting better than wild-type AAV9 aggregated (in all brain regions, and across both CBH and hSyn promoters) brain transduction and preserving at least 3 of the mutations of the mutation set of VAR-B1, these additional mutations (beyond the motifs described above) were present at positions corresponding to over 65 unique positions relative to SEQ ID NO:1, with a variety of permitted amino acids at each of these positions.

Without being bound by theory, these data support the finding that inclusion of these motifs into capsid polypeptides and virus particles comprising such capsid polypeptides results in, for example, increased transduction and expression of a heterologous transgene included in such virus particle, relative to an otherwise similar virus particle comprising the capsid polypeptides of wild-type AAV9 (e.g., the capsid polypeptides of SEQ ID NO:1). These data establish that virus particles comprising a capsid polypeptide comprising any one of the mutations or motifs described herein have unexpectedly marked improvements over otherwise similar virus particles comprising a capsid consisting of wild-type AAV9 capsid polypeptides, including in transduction of a heterologous transgene packaged therein in cells of the central nervous system (“CNS”), for example, neurons. Such capsid polypeptides are thus contemplated for use in gene therapies, e.g., virus particles and compositions comprising the same, for use in the treatment of a subject in need thereof, for example, a human, for example for the treatment of a disorder of the CNS.

Upon assessing the other properties of VAR-1 to VAR-62, variants emerged with transduction and biodistribution profiles which suggest the potential for use in gene therapy therapeutics for delivering transgenes to target cell types of relevance and/or for treating CNS disorders. Overall, virus particles comprising these variants exhibited the highest levels of transduction from any brain region in the basal ganglia and the lowest fold-improvement over wild-type AAV9 in the brainstem, but in nearly all regions these variants exhibited transduction rates significantly higher than wild-type AAV9.

Without being bound by theory, treatment of many CNS disorders would benefit from efficient delivery to multiple brain regions. These include diseases, such as, for example, progressive supranuclear palsy (PSP), Alzheimer's disease, Rett syndrome, Dravet syndrome, multiple systems atrophy (MSA), sanfilippo syndrome, fragile X disease and others. In addition, treatments for Huntington's disease would benefit from delivery to all brain regions but in particular the striatum and cerebellum. Many of the capsid variants described herein meet this profile, with aggregate brain transduction across all brain regions achieving more than 200-fold higher levels of transduction than wild-type AAV9 (VAR-2). Other capsid variants from amongst this set exhibiting greater than 150-fold improvement in aggregated brain transduction relative to wild-type AAV9 include VAR-2, VAR-4, VAR-5, VAR-15, VAR-18, VAR-14, VAR-23, VAR-24, VAR-26, VAR-28, VAR-38 and VAR-39. Without being bound by theory, capsids which also exhibit substantial improvement (>100-fold over wild-type AAV9) in transduction of the hippocampus, such as VAR-24, VAR-39, VAR-15 and VAR-10 are also of particular interest for treatment of CNS conditions presenting in the hippocampus, among other areas.

Without being bound by theory, treatment of other diseases of the CNS would benefit from virus particles capable of high efficiency transduction across multiple brain regions and the spinal cord. One example of such a disease is Tay-Sachs disease. Variants described herein exhibit these properties, with VAR-13 and VAR-39 exhibiting both 100-fold improved transduction efficiency across aggregated brain tissue and 100-fold improved transduction efficiency of spinal cord, relative to wild-type AAV9. VAR-3, VAR-7, VAR-15, VAR-18, VAR-23, VAR-25, VAR-37, VAR-44 and VAR-50 all have at least 90-fold improved transduction in both aggregated brain and spinal cord tissue. Without being bound by theory, virus particles described herein comprising the capsid polypeptides described herein, including those described above, are thus useful for the treatment of CNS disorders presenting across the brain and spinal cord, such as for example, Tay-Sachs disease.

Without being bound by theory, treatment of other diseases of the CNS would benefit from virus particles capable of high efficiency transduction specifically in the cerebellum and spinal cord. One example of such a disease is spinocerebellar ataxia. Variants described herein exhibit these properties, with VAR-13 and VAR-39 exhibiting both 100-fold improved transduction efficiency in cerebellum and spinal cord, relative to wild-type AAV9. In addition, VAR-3, VAR-7, VAR-15, VAR-18, VAR-25, VAR-37, VAR-44 and VAR-50 all have at least 90-fold improved transduction in both cerebellum and spinal cord tissue. Without being bound by theory, virus particles described herein comprising the capsid polypeptides described herein, including those described above, are thus useful for the treatment of CNS disorders presenting in the cerebellum and spinal cord, such as for example, spinocerebellar ataxia.

Without being bound by theory, treatment of other diseases of the CNS would benefit from virus particles capable of high efficiency transduction throughout all cortical regions. One example of such a disease is frontotemporal dementia (FTD). Variants described herein exhibit widespread increased transduction of the cortex, with VAR-2 exhibiting greater than 200-fold improved transduction efficiency in both the temporal cortex and frontal lobe of the cortex, relative to wild-type AAV9. In addition, VAR-5, VAR-8, VAR-15, VAR-17, VAR-18, VAR-26, VAR-28, and VAR-30 all have at least 150-fold improved transduction in both the temporal cortex and frontal lobe. Without being bound by theory, virus particles described herein comprising the capsid polypeptides described herein, including those described above, are thus useful for the treatment of CNS disorders presenting in the cortex, such as for example, FTD.

Without being bound by theory, treatment of other diseases of the CNS would benefit from virus particles capable of high efficiency transduction in the frontal cortex, brainstem and spinal cord. Examples of such diseases are ALS (all regions) and spinal muscular atrophy (brainstem and spinal cord). Variants described herein exhibit widespread increased transduction of the frontal cortex, brainstem and spinal cord, such as, for example, VAR-3, VAR-7, VAR-13, VAR-15, VAR-18, and VAR-25 all exhibiting at least 90-fold improved transduction in all of these regions. Other variants which exhibit transduction profiles applicable to such disorders include VAR-1 and VAR-8, which each have at least 80-fold increased transduction of the brainstem and spinal cord, with greater than 200-fold increased transduction in the forebrain. Without being bound by theory, virus particles described herein comprising the capsid polypeptides described herein, including those described above, are thus useful for the treatment of CNS disorders presenting in the frontal cortex, brainstem and/or spinal cord, such as for example, ALS or spinal muscular atrophy.

Without being bound by theory, treatment of other diseases of the CNS would benefit from virus particles capable of high efficiency transduction in the midbrain and frontal cortex. An example of such a disease is Parkinson's disease. Variants described herein exhibit widespread increased transduction of the frontal cortex and midbrain, such as, for example, VAR-2, VAR-4, VAR-5, VAR-8, VAR-13, VAR-18, VAR-24, VAR-28 and VAR-33, all exhibiting at least 200-fold improved transduction over wild-type AAV9 in both of these regions. Without being bound by theory, virus particles described herein comprising the capsid polypeptides described herein, including those described above, are thus useful for the treatment of CNS disorders presenting in the frontal cortex and midbrain, such as for example, Parkinson's disease.

Without being bound by theory, treatment of other diseases of the CNS would benefit from virus particles capable of high efficiency transduction in the basal ganglia and cerebellum. An example of such a disease is Huntington's disease. Variants described herein exhibit widespread increased transduction of both the basal ganglia and cerebellum, with VAR-2 and VAR-39 exhibiting at least 200-fold increased transduction of these areas relative to wild-type AAV9 (VAR-2 exhibits over 400-fold increased transduction in the basal ganglia). In addition, several other variants described herein exhibit over 300-fold improved basal ganglia transduction and 100-fold improved cerebellum transduction (e.g., VAR-2, VAR-5, VAR-14, VAR-18, VAR-24, VAR-26, VAR-28, and VAR-40). Without being bound by theory, virus particles described herein comprising the capsid polypeptides described herein, including those described above, are thus useful for the treatment of CNS disorders presenting in the basal ganglia and cerebellum, such as for example, Huntington's disease.

Other transduction, biodistribution and production profiles of interest associated with each of the variants described herein and as reported in the Tables also support the usefulness of the capsid polypeptides described herein in gene therapies targeting disorders of the CNS.

### 7.6. Example 6: Skeletal Muscle Biodistribution and Transduction Properties of Viral Particles with Variant Capsid Polypeptides

### 7.6.1. Overview

The skeletal muscle biodistribution and transduction properties viral particles containing the capsid polypeptides of VAR-1 to VAR-62 described in Example 5 in skeletal muscle using the procedures described in Section 7.5.2.3. The skeletal muscle biodistribution and transduction properties of virus particles containing the capsid polypeptides of VAR-1 to VAR-62 (described in Example 5) and VAR-B1 to VAR-B127 (described in Example 4) were compared and ranked.

### 7.6.2. Results: VAR-1 to VAR-62 Skeletal Muscle Biodistribution and Transduction

Almost all VAR-1 to VAR-62 increased biodistribution in skeletal muscle as compared to capsid polypeptides of SEQ ID NO:1. Results of this analysis are summarized in Table 28. Additionally, a large number of the VAR-1 to VAR-62 capsids increased transduction as compared to capsid polypeptides of SEQ ID NO:1. Results of this analysis are summarized in Table 29.

TABLE 28
Fold change in biodistribution of virus particles comprising the
indicated variant capsid polypeptide, relative to the biodistribution
of virus particles comprising WT AAV9 for muscle tissue, with the
95% confidence interval width shown in parentheses following each
value. CBh indicates measurements from virus particles comprising
genomes with the cap gene under the control of the CBh promoter.
hSyn indicates virus particles comprising genomes with the cap
gene under the control of the hSyn promoter. “Aggregated”
in relation to a promoter is an average across the CBh.

| | ORGAN | | |
|---|---|---|---|
| | Skeletal Muscle | Skeletal Muscle | Skeletal Muscle |
| | PROMOTER | | |
| VAR# | CBh | hSyn | Aggregated |
| VAR-1 | 5.17 (3.6) | 7.44 (9.53) | 5.11 (3.2) |
| VAR-2 | 3.73 (4.9) | 3.2 (4.17) | 3.83 (4.05) |
| VAR-3 | 6.66 (5.32) | 3.8 (5.77) | 5.82 (3.51) |
| VAR-4 | 4.67 (3.37) | 5.8 (6.66) | 4.82 (3.06) |
| VAR-5 | 4.4 (3.87) | 6.35 (6.42) | 5.11 (3.24) |
| VAR-6 | 6.24 (3.8) | 8.07 (5.22) | 6.96 (3.27) |
| VAR-7 | 6.35 (3.02) | 6.59 (5.6) | 6.26 (3.07) |
| VAR-8 | 6.99 (5.66) | 2.57 (4.25) | 5.68 (4.3) |
| VAR-9 | 5.29 (4.22) | 2.68 (3.79) | 4.55 (3.27) |
| VAR-10 | 4.27 (3.92) | 4.73 (5.64) | 4.81 (3.33) |
| VAR-11 | 4.06 (3.64) | 0.74 (1.88) | 3.34 (3.51) |
| VAR-12 | 6.35 (4.41) | 7.16 (6.39) | 6.6 (3.23) |
| VAR-13 | 5.45 (3.58) | 4.3 (3.45) | 5.38 (3.35) |
| VAR-14 | 3.37 (4.21) | 4 (4.72) | 4.25 (4.17) |
| VAR-15 | 6.84 (3.68) | 7.91 (5.2) | 7.08 (2.42) |
| VAR-16 | 5.25 (3.05) | 3.03 (3.43) | 4.65 (2.2) |
| VAR-17 | 6.92 (4.46) | 5.01 (6.63) | 6.37 (3.39) |
| VAR-18 | 9.1 (6.35) | 5.91 (5.64) | 8.52 (5.01) |
| VAR-19 | 7.19 (3.65) | 4.09 (5.34) | 6.26 (3.33) |
| VAR-20 | 4.7 (3.89) | 4.32 (5.18) | 4.59 (3.51) |
| VAR-21 | 1.87 (4.34) | 2.59 (6.27) | 2.29 (3.66) |
| VAR-22 | 3.83 (2.5) | 5.39 (5.07) | 4.13 (2.26) |
| VAR-23 | 4.4 (5.58) | 2.96 (6.16) | 4.04 (4.49) |
| VAR-24 | 5.55 (2.97) | 6.82 (4.22) | 6.42 (2.94) |
| VAR-25 | 6.03 (3.46) | 5.72 (3.39) | 6.3 (2.89) |
| VAR-26 | 7.08 (6.09) | 3.5 (6.48) | 6.06 (4.35) |
| VAR-27 | 5.43 (4.58) | 4.3 (3.9) | 5.45 (3.78) |
| VAR-28 | 3.29 (3.71) | 6.04 (5.61) | 5.7 (3.44) |
| VAR-29 | 3.09 (2.97) | 2.66 (3.28) | 3.16 (2.25) |
| VAR-30 | 6.14 (5.3) | 4.62 (5.26) | 6.05 (3.78) |
| VAR-31 | 5.52 (3.84) | 5.63 (4.9) | 5.64 (3.31) |
| VAR-32 | 5.46 (5.43) | 3.57 (6.36) | 4.94 (4.32) |
| VAR-33 | 4.2 (3.24) | 2.11 (2.37) | 3.61 (2.15) |
| VAR-34 | 4.52 (3.67) | 6.82 (5.9) | 5.14 (2.36) |
| VAR-35 | 2.02 (2.53) | 4.73 (4.64) | 3.27 (2.91) |
| VAR-36 | 2.87 (3.44) | 3.35 (4.93) | 2.94 (2.64) |
| VAR-37 | 6.05 (3.53) | 4.05 (3.25) | 5.51 (2.5) |
| VAR-38 | 6.53 (2.17) | 6.53 (0.19) | 7.28 (0.11) |
| VAR-39 | 6.91 (3.6) | 4.24 (3.79) | 6.31 (3.21) |
| VAR-40 | 6.87 (4.41) | 4.89 (3.66) | 6.48 (3.32) |
| VAR-41 | 4.38 (4.9) | 4.87 (6.15) | 4.83 (3.87) |
| VAR-42 | 7.25 (6.5) | 6.82 (8.49) | 7.15 (6) |
| VAR-43 | 8.84 (5.18) | 5.95 (5.84) | 8.06 (4.42) |
| VAR-44 | 5.73 (2.94) | 6.43 (2.56) | 6.42 (1.6) |
| VAR-45 | 4.85 (5.98) | 8.29 (10.66) | 5.48 (5.15) |
| VAR-46 | 5.02 (3.63) | 3.45 (4.89) | 4.61 (2.97) |
| VAR-47 | 4.89 (5.82) | 1.45 (2.94) | 3.76 (3.96) |
| VAR-48 | 5.44 (5.46) | 5.82 (7.04) | 5.62 (4.39) |
| VAR-49 | 2.29 (3.11) | 0.27 (0.58) | 1.74 (2.37) |
| VAR-50 | 7.89 (4.36) | 7.13 (0.29) | 8.62 (2.02) |
| VAR-51 | 5.92 (3.3) | 6.11 (3.03) | 6.25 (1.99) |
| VAR-52 | 3.53 (2.67) | 2.5 (4.28) | 3.16 (2.35) |
| VAR-53 | 2.73 (3.56) | 4.17 (5.57) | 3.87 (3.35) |
| VAR-54 | 4.53 (5.03) | 1.63 (2.97) | 3.54 (3.95) |
| VAR-55 | 5.25 (3.72) | 3.69 (4.28) | 4.9 (3.34) |
| VAR-56 | 6.03 (3.43) | 2.96 (3.96) | 5.15 (2.73) |
| VAR-57 | 3.45 (3.64) | 3.74 (4.01) | 3.84 (3.03) |
| VAR-58 | 2.69 (3.96) | 3.13 (3.89) | 3.53 (4.09) |
| VAR-59 | 9.76 (4.46) | 6.97 (6.15) | 9.05 (4.07) |
| VAR-60 | 3.23 (1.68) | 2.22 (3.13) | 2.87 (1.57) |
| VAR-61 | 4.75 (4.24) | 2.55 (3.78) | 4.14 (3.11) |
| VAR-62 | 5.72 (3.65) | 7.13 (6.56) | 6.13 (2.46) |

TABLE 29
Fold change in transduction of virus particles comprising the
indicated variant capsid polypeptide, relative to the transduction
of virus particles comprising wild-type AAV9 in muscle tissues,
with the 95% confidence interval width shown in parentheses
following each value. CBh indicates measurements from virus
particles comprising genomes with the cap gene under the control
of the CBh promoter. hSyn indicates virus particles comprising
genomes with the cap gene under the control of the hSyn promoter.
“Aggregated” in relation to a promoter is an average
across the CBh and hSyn promoters. “N/D” indicates variant
transduction was not detected in the indicated sample.

| | ORGAN | | |
|---|---|---|---|
| | Skeletal Muscle | Skeletal Muscle | Skeletal Muscle |
| | PROMOTER | | |
| VAR# | CBh | hSyn | Aggregated |
| VAR-1 | 2.39 (1.42) | 10 (10.22) | 2.86 (1.5) |
| VAR-2 | 1.13 (1.18) | N/D | 0.89 (1.03) |
| VAR-3 | 2.13 (1.26) | 1.75 (3.58) | 2.13 (1.32) |
| VAR-4 | 1.61 (1.08) | N/D | 1.5 (0.93) |
| VAR-5 | 2.53 (1.51) | 1.14 (2.33) | 2.34 (1.34) |
| VAR-6 | 1.72 (0.95) | 1.54 (2.3) | 1.68 (0.96) |
| VAR-7 | 2.46 (1.06) | 2.4 (3.11) | 2.49 (1.03) |
| VAR-8 | 1.42 (1.06) | 1.29 (2.64) | 1.38 (1.12) |
| VAR-9 | 2.52 (1.44) | 0.92 (1.87) | 2.29 (1.41) |
| VAR-10 | 1.29 (0.98) | 1.67 (2.56) | 1.3 (1.04) |
| VAR-11 | 2.06 (1.35) | N/D | 2.08 (1.32) |
| VAR-12 | 1.88 (1.03) | 1.09 (2.25) | 1.8 (1.15) |
| VAR-13 | 2.86 (1.67) | 2.12 (2.66) | 2.61 (1.3) |
| VAR-14 | 2.78 (2.03) | N/D | 1.97 (1.31) |
| VAR-15 | 2.02 (1.13) | 1.48 (2.33) | 1.98 (0.98) |
| VAR-16 | 2.66 (1.5) | 0.56 (1.14) | 2.32 (1.43) |
| VAR-17 | 1.5 (1.07) | N/D | 1.35 (1.12) |
| VAR-18 | 3.96 (2.03) | 1.95 (2.61) | 3.34 (1.74) |
| VAR-19 | 3.46 (1.53) | 2.71 (4.33) | 3.49 (1.48) |
| VAR-20 | 2.46 (1.55) | 1.38 (2.81) | 2.35 (1.28) |
| VAR-21 | 1.9 (2.57) | N/D | 1.56 (2.1) |
| VAR-22 | 1.49 (0.87) | 2.53 (3.34) | 1.59 (0.75) |
| VAR-23 | 2.37 (1.86) | 2.02 (4.18) | 2.28 (1.56) |
| VAR-24 | 1.66 (0.96) | 0.99 (1.53) | 1.51 (0.92) |
| VAR-25 | 1.94 (1.09) | 0.54 (1.1) | 1.66 (1.03) |
| VAR-26 | 1.03 (1.18) | N/D | 0.91 (0.93) |
| VAR-27 | 1.31 (1.03) | N/D | 1 (0.79) |
| VAR-28 | 1.88 (1.36) | 0.55 (1.1) | 1.38 (1.02) |
| VAR-29 | 0.24 (0.48) | N/D | 0.19 (0.38) |
| VAR-30 | 0.53 (0.71) | 2.28 (3.03) | 0.81 (0.82) |
| VAR-31 | 1.98 (1.37) | 0.92 (1.86) | 1.85 (1.14) |
| VAR-32 | 2.02 (1.6) | N/D | 1.8 (1.38) |
| VAR-33 | 1.24 (0.81) | 1.42 (1.87) | 1.22 (0.64) |
| VAR-34 | 1.01 (0.57) | 0.85 (1.71) | 1 (0.67) |
| VAR-35 | 2.62 (1.43) | 0.78 (1.58) | 2.26 (1.26) |
| VAR-36 | 1.64 (1.2) | 3.11 (4.59) | 1.78 (1.26) |
| VAR-37 | 2.33 (1.11) | 4.2 (3.76) | 2.52 (0.99) |
| VAR-38 | 2.35 (1.36) | 3.33 (0.67) | 2.38 (0.75) |
| VAR-39 | 2.91 (1.35) | 2.08 (2.19) | 2.6 (1.52) |
| VAR-40 | 2.22 (1.13) | 1.85 (2.51) | 2.1 (1.06) |
| VAR-41 | 0.39 (0.61) | N/D | 0.32 (0.48) |
| VAR-42 | 2.94 (2.12) | 4.67 (9.02) | 3.11 (2.21) |
| VAR-43 | 2.05 (1.19) | 1.03 (2.11) | 1.92 (1.02) |
| VAR-44 | 2.49 (1.16) | 1.43 (1.46) | 2.23 (0.95) |
| VAR-45 | 2.88 (2.28) | N/D | 2.7 (2.13) |
| VAR-46 | 1.32 (0.94) | N/D | 1.16 (0.74) |
| VAR-47 | 0.64 (1.28) | 3.28 (4.77) | 1.03 (1.05) |
| VAR-48 | 1.39 (1.2) | 3.26 (5.15) | 1.59 (1.1) |
| VAR-49 | 0.86 (0.9) | N/D | 0.78 (0.8) |
| VAR-50 | 2.64 (1.21) | 2.08 (1.79) | 2.31 (0.97) |
| VAR-51 | 2.82 (0.97) | 0.82 (1.65) | 2.49 (0.93) |
| VAR-52 | 0.91 (0.62) | 4.45 (5.88) | 1.17 (0.8) |
| VAR-53 | N/D | 2.1 (3.19) | 0.41 (0.8) |
| VAR-54 | 0.73 (0.98) | 0.94 (1.9) | 0.72 (0.73) |
| VAR-55 | 2.02 (1.32) | 1.87 (2.92) | 1.96 (1.35) |
| VAR-56 | 1.44 (0.83) | 1.34 (2.67) | 1.39 (0.86) |
| VAR-57 | 1.06 (0.92) | 2.36 (3.15) | 1.22 (0.87) |
| VAR-58 | 3.45 (2.61) | N/D | 2.26 (1.52) |
| VAR-59 | 4.55 (2.06) | 0.99 (2.02) | 4.1 (1.87) |
| VAR-60 | 1.47 (0.84) | 1.82 (1.93) | 1.54 (0.81) |
| VAR-61 | 0.63 (0.66) | 1.88 (2.69) | 0.79 (0.68) |
| VAR-62 | 2.9 (1.49) | 3.85 (3.81) | 3 (1.16) |

### 7.6.3. Results: Ranked Skeletal Muscle Biodistribution and Transduction Properties of Variant Capsid Polypeptides

The skeletal muscle biodistribution of viral particles containing capsids of VAR-Bi to VAR-B127 and VAR-i through VAR-62 (using CBh promoter for the variants VAR-Bi to VAR-B127 as set forth in Table 18 and as set forth in Table 28 for VAR-i through VAR-62) was ranked from highest to lowest biodistribution relative to viral particles containing capsid polypeptides of SEQ ID NO:11). results of this analysis are set forth in Table 30.

TABLE 30
Compiled Cbh biodistribution x mutation data (VAR-B1 through VAR-B127 and VAR1-VAR62)

| Var# | x-fold vs AAV9 | Mutations |
|---|---|---|
| VAR-59 | 9.76 | [‘E575W’, ‘H584N’, ‘T593V’, ‘W595A’, ‘V596L’, ‘N598S’, ‘I601V’] |
| VAR-18 | 9.1 | [‘G549T’, ‘Q579T’, ‘T593V’, ‘W595A’, ‘V596L’, ‘N598S’, ‘I601V’] |
| VAR-43 | 8.84 | [‘Q579V’, ‘Q588N’, ‘T593V’, ‘W595A’, ‘V596L’, ‘N598T’, ‘I601V’] |
| VAR-50 | 7.89 | [‘Q579V’, ‘A587S’, ‘T593S’, ‘W595A’, ‘V596L’, ‘N598S’, ‘I601V’] |
| VAR-42 | 7.25 | [‘Q579T’, ‘H584N’, ‘T593V’, ‘W595A’, ‘V596L’, ‘N598S’] |
| VAR-19 | 7.19 | [‘Q579V’, ‘T582I’, ‘H584N’, ‘T593V’, ‘W595A’, ‘V596L’, ‘N598S’] |
| VAR-26 | 7.08 | [‘M559A’, ‘Q579V’, ‘Q592S’, ‘T593V’, ‘W595A’, ‘V596L’, ‘N598T’, ‘I601V’] |
| VAR-8 | 6.99 | [‘Y577H’, ‘Q579V’, ‘A581C’, ‘T593V’, ‘W595A’, ‘V596L’, ‘N598S’, ‘I601V’] |
| VAR-17 | 6.92 | [‘M559C’, ‘Q579V’, ‘T593A’, ‘W595A’, ‘V596L’, ‘N598S’] |
| VAR-39 | 6.91 | [‘M559Q’, ‘Q579V’, ‘T582I’, ‘T593V’, ‘W595A’, ‘V596L’, ‘N598S’] |
| VAR-40 | 6.87 | [‘Q579V’, ‘T593V’, ‘W595A’, ‘V596L’, ‘N598T’, ‘I601V’] |
| VAR-15 | 6.84 | [‘M559Q’, ‘Q579V’, ‘H584Q’, ‘T593V’, ‘W595A’, ‘V596L’, ‘N598S’, ‘I601V’] |
| VAR-3 | 6.66 | [‘Q579V’, ‘Q588S’, ‘T593A’, ‘W595A’, ‘V596L’, ‘N598S’, ‘I601V’] |
| VAR-38 | 6.53 | [‘G549H’, ‘Q579T’, ‘T593V’, ‘W595A’, ‘V596L’, ‘N598S’, ‘I601V’] |
| VAR-12 | 6.35 | [‘M559V’, ‘Q579V’, ‘T593S’, ‘W595A’, ‘V596L’, ‘N598S’, ‘I601V’] |
| VAR-7 | 6.35 | [‘Q579V’, ‘Q588G’, ‘T593S’, ‘W595A’, ‘V596L’, ‘N598S’, ‘I601V’] |
| VAR-6 | 6.24 | [‘M559N’, ‘Q579V’, ‘T593S’, ‘W595A’, ‘V596L’, ‘N598S’, ‘I601V’] |
| VAR-30 | 6.14 | [‘K557N’, ‘Q579V’, ‘H584L’, ‘A589T’, ‘Q592S’, ‘T593V’, ‘W595A’, ‘V596L’, ‘N598S’, |
| | | ‘I601V’] |
| VAR-37 | 6.05 | [‘G549I’, ‘Q579V’, ‘T593S’, ‘W595A’, ‘V596L’, ‘N598S’, ‘I601V’] |
| VAR-25 | 6.03 | [‘T561S’, ‘Q579V’, ‘T593A’, ‘W595A’, ‘V596L’, ‘N598S’, ‘I601V’] |
| VAR-56 | 6.03 | [‘Q579S’, ‘H584L’, ‘T593V’, ‘W595Y’, ‘V596L’, ‘N598S’, ‘I601V’] |
| VAR-51 | 5.92 | [‘Q579T’, ‘Q588N’, ‘A589T’, ‘T593V’, ‘W595A’, ‘V596L’, ‘N598S’, ‘I601V’] |
| VAR-44 | 5.73 | [‘Q579V’, ‘Q588N’, ‘T593A’, ‘W595A’, ‘V596L’, ‘N598S’, ‘I601V’] |
| VAR-62 | 5.72 | [‘E575S’, ‘H584M’, ‘T593V’, ‘W595A’, ‘V596L’, ‘N598S’] |
| VAR-24 | 5.55 | [‘Q579V’, ‘H584I’, ‘Q592S’, ‘T593V’, ‘W595A’, ‘V596L’, ‘N598S’, ‘I601V’] |
| VAR-31 | 5.52 | [‘Q579T’, ‘H584L’, ‘T593V’, ‘W595A’, ‘V596L’, ‘N598S’] |
| VAR-32 | 5.46 | [‘Q579V’, ‘Q588T’, ‘T593A’, ‘W595A’, ‘V596L’, ‘N598S’] |
| VAR-13 | 5.45 | [‘I560Q’, ‘Q579T’, ‘T593V’, ‘W595A’, ‘V596L’, ‘N598S’, ‘I601V’] |
| VAR-48 | 5.44 | [‘Q579T’, ‘S586Q’, ‘A587S’, ‘T593V’, ‘W595A’, ‘V596L’, ‘N598S’, ‘I601V’] |
| VAR-27 | 5.43 | [‘Y577T’, ‘Q579V’, ‘Q592S’, ‘T593V’, ‘W595A’, ‘V596L’, ‘N598S’, ‘I601V’] |
| VAR-9 | 5.29 | [‘I560L’, ‘Q579V’, ‘A589T’, ‘T593A’, ‘W595A’, ‘V596L’, ‘N598S’] |
| VAR-55 | 5.25 | [‘Q579V’, ‘H584N’, ‘T593V’, ‘W595A’, ‘V596L’, ‘N598S’, ‘I601V’] |
| VAR-16 | 5.25 | [‘Q579V’, ‘Q592I’, ‘T593R’, ‘W595A’, ‘V596L’, ‘N598S’] |
| VAR-1 | 5.17 | [‘Q579V’, ‘A589T’, ‘T593A’, ‘W595A’, ‘V596L’, ‘N598S’, ‘I601V’] |
| VAR-B54 | 5.07 | [“Q579V”, “T593V”, “W595A”, “V596L”, “N598S”, “I601A”] |
| VAR-46 | 5.02 | [‘R550L’, ‘Q579T’, ‘T593V’, ‘W595A’, ‘V596L’, ‘N598S’, ‘I601V’] |
| VAR-47 | 4.89 | [‘K557N’, ‘Q579V’, ‘Q588N’, ‘T593A’, ‘W595A’, ‘V596L’, ‘N598S’, ‘I601V’, ‘D609N’] |
| VAR-45 | 4.85 | [‘Q579T’, ‘Q588N’, ‘A589T’, ‘Q592N’, ‘T593V’, ‘W595A’, ‘V596L’, ‘N598S’, ‘I601V’] |
| VAR-61 | 4.75 | [‘M559T’, ‘Q579V’, ‘A587S’, ‘Q588N’, ‘T593S’, ‘W595A’, ‘V596L’, ‘N598S’] |
| VAR-20 | 4.7 | [‘G549A’, ‘Q579V’, ‘T593S’, ‘W595A’, ‘V596L’, ‘N598S’, ‘I601V’] |
| VAR-4 | 4.67 | [‘G549Y’, ‘Q579T’, ‘T593V’, ‘W595A’, ‘V596L’, ‘N598S’, ‘I601V’] |
| VAR-B61 | 4.57 | [“Q592I”, “T593V”, “V596L”, “N598S”, “I601A”] |
| VAR-B94 | 4.55 | [“Q579V”, “Q592I”, “V596L”] |
| VAR-54 | 4.53 | [‘S576W’, ‘Q579T’, ‘A581N’, ‘T593V’, ‘W595A’, ‘V596L’, ‘N598S’, ‘I601V’] |
| VAR-34 | 4.52 | [‘G549F’, ‘Q579T’, ‘T593V’, ‘W595A’, ‘V596L’, ‘N598S’, ‘I601V’] |
| VAR-23 | 4.4 | [‘T561S’, ‘Q579V’, ‘H584I’, ‘Q592S’, ‘T593V’, ‘W595A’, ‘V596L’, ‘N598S’, ‘I601V’] |
| VAR-5 | 4.4 | [‘M559S’, ‘Q579T’, ‘H584I’, ‘Q592I’, ‘T593V’, ‘W595A’, ‘V596L’, ‘N598S’] |
| VAR-41 | 4.38 | [‘Y577T’, ‘Q579V’, ‘A589S’, ‘Q592A’, ‘T593V’, ‘W595A’, ‘V596L’, ‘N598S’, ‘I601V’] |
| VAR-10 | 4.27 | [‘M559A’, ‘Q579V’, ‘H584M’, ‘Q592S’, ‘T593V’, ‘W595A’, ‘V596L’, ‘N598S’, ‘I601V’] |
| VAR-B1 | 4.22 | [‘Q579V’, ‘Q592l’, ‘T593V’, ‘W595A’, ‘V596L’, ‘N598S’, ‘I601A’] |
| VAR-33 | 4.2 | [‘Y577H’, ‘Q579V’, ‘T582M’, ‘Q592S’, ‘T593V’, ‘W595A’, ‘V596L’, ‘N598S’, ‘I601V’] |
| VAR-11 | 4.06 | [‘Q579V’, ‘Q588N’, ‘T593A’, ‘W595A’, ‘V596L’, ‘N598S’] |
| VAR-B73 | 3.95 | [“T593V”, “V596L”, “I601A”] |
| VAR-22 | 3.83 | [‘R550K’, ‘Q579V’, ‘A589S’, ‘T593A’, ‘W595A’, ‘V596L’, ‘N598S’] |
| VAR-2 | 3.73 | [‘D551E’, ‘Q579T’, ‘Q592S’, ‘T593V’, ‘W595A’, ‘V596L’, ‘N598S’, ‘I601V’] |
| VAR-B87 | 3.57 | [“Q579V”, “W595A”, “V596L”, “N598S”, “I601A”] |
| VAR-52 | 3.53 | [‘G549N’, ‘Q579T’, ‘A587S’, ‘A589T’, ‘T593V’, ‘W595A’, ‘V596L’, ‘N598S’] |
| VAR-B46 | 3.5 | [“Q579V”, “Q592I”, “W595A”, “N598S”, “I601A”] |
| VAR-57 | 3.45 | [‘M559I’, ‘Q579S’, ‘Q592A’, ‘V596L’, ‘N598S’, ‘I601A’] |
| VAR-14 | 3.37 | [‘Q579T’, ‘Q588N’, ‘A589T’, ‘Q592S’, ‘T593V’, ‘W595A’, ‘V596L’, ‘N598S’, ‘I601V’] |
| VAR-B74 | 3.33 | [“Q579V”, “T593V”, “V596L”, “N598S”, “I601A”] |
| VAR-28 | 3.29 | [‘V553S’, ‘Q579T’, ‘T593V’, ‘W595A’, ‘V596L’, ‘N598S’, ‘I601V’] |
| VAR-60 | 3.23 | [‘M559A’, ‘Q579T’, ‘S586Q’, ‘A587T’, ‘T593V’, ‘W595A’, ‘V596L’, ‘N598S’] |
| VAR-29 | 3.09 | [‘Q579T’, ‘A587H’, ‘Q588N’, ‘A589T’, ‘Q592l’, ‘T593V’, ‘W595A’, ‘V596L’, ‘N598S’, |
| | | ‘I601V’] |
| VAR-36 | 2.87 | [‘R550K’, ‘Q579V’, ‘A589S’, ‘T593S’, ‘W595A’, ‘V596L’, ‘N598S’] |
| VAR-B104 | 2.83 | [“Q592I”, “T593V”, “W595A”, “V596L”] |
| VAR-53 | 2.73 | [‘K557L’, ‘M559Q’, ‘Q579V’, ‘T593A’, ‘W595A’, ‘V596L’, ‘N598S’] |
| VAR-B100 | 2.72 | [“Q592I”, “T593V”, “W595A”, “V596L”, “N598S”, “I601A”] |
| VAR-B83 | 2.71 | [“V596L”, “N598S”, “I601A”] |
| VAR-58 | 2.69 | [‘Q579S’, ‘A591P’, ‘V596L’, ‘N598S’, ‘I601A’] |
| VAR-B80 | 2.56 | [“T593V”, “V596L”, “N598S”, “I601A”] |
| VAR-B15 | 2.52 | [“W595A”, “N598S”] |
| VAR-B25 | 2.38 | [“Q579V”, “Q592I”, “T593V”, “W595A”, “N598S”] |
| VAR-B86 | 2.3 | [“Q579V”, “V596L”] |
| VAR-49 | 2.29 | [‘Q579T’, ‘Q588N’, ‘A589T’, ‘Q592A’, ‘T593V’, ‘W595A’, ‘V596L’, ‘N598S’] |
| VAR-B116 | 2.24 | [“Q579V”, “Q592I”, “W595A”, “V596L”, “N598S”, “I601A”] |
| VAR-B112 | 2.2 | [“W595A”, “V596L”, “N598S”] |
| VAR-B97 | 2.17 | [“Q579V”, “W595A”, “V596L”, “I601A”] |
| VAR-B66 | 2.14 | [“Q579V”, “Q592I”, “T593V”, “W595A”, “V596L”, “I601A”] |
| VAR-B43 | 2.04 | [“T593V”, “N598S”, “I601A”] |
| VAR-35 | 2.02 | [‘S576W’, ‘Q579V’, ‘A587S’, ‘A589T’, ‘T593V’, ‘W595A’, ‘V596L’, ‘N598S’] |
| VAR-B102 | 1.98 | [“Q579V”, “Q592I”, “T593V”, “V596L”, “N598S”, “I601A”] |
| VAR-B89 | 1.88 | [“T593V”, “W595A”, “V596L”, “N598S”, “I601A”] |
| VAR-21 | 1.87 | [‘R550N’, ‘Y577T’, ‘Q579V’, ‘Q592S’, ‘T593V’, ‘W595A’, ‘V596L’, ‘N598S’, ‘I601V’] |
| VAR-B110 | 1.74 | [“Q592I”, “T593V”, “V596L”] |
| VAR-B67 | 1.73 | [“Q579V”, “V596L”, “N598S”, “I601A”] |
| VAR-B77 | 1.7 | [“Q579V”, “T593V”, “W595A”, “V596L”, “I601A”] |
| VAR-B60 | 1.7 | [“T593V”, “V596L”] |
| VAR-B50 | 1.67 | [“Q579V”, “Q592I”, “N598S”] |
| VAR-B39 | 1.66 | [“Q579V”, “Q592I”, “T593V”, “N598S”, “I601A”] |
| VAR-B111 | 1.66 | [“Q579V”, “Q592I”, “T593V”, “V596L”, “I601A”] |
| VAR-B95 | 1.6 | [“T593V”, “V596L”, “N598S”] |
| VAR-B40 | 1.54 | [“T593V”, “W595A”, “N598S”, “I601A”] |
| VAR-B69 | 1.53 | [“Q579V”, “Q592I”, “W595A”, “V596L”, “I601A”] |
| VAR-B12 | 1.53 | [“Q592I”] |
| VAR-B42 | 1.53 | [“Q592I”, “T593V”, “W595A”, “N598S”] |
| VAR-B93 | 1.37 | [“Q592I”, “T593V”, “W595A”, “V596L”, “N598S”] |
| VAR-B90 | 1.31 | [“Q579V”, “Q592I”, “W595A”, “V596L”, “N598S”] |
| VAR-B20 | 1.31 | [“Q592I”, “W595A”, “N598S”, “I601A”] |
| VAR-B68 | 1.3 | [“Q579V”, “W595A”, “V596L”] |
| VAR-B38 | 1.3 | [“Q579V”, “Q592I”, “N598S”, “I601A”] |
| VAR-B85 | 1.29 | [“V596L”, “N598S”] |
| VAR-B51 | 1.29 | [“Q592I”, “T593V”, “W595A”] |
| VAR-B16 | 1.29 | [“W595A”, “I601A”] |
| VAR-B26 | 1.28 | [“Q579V”, “W595A”, “N598S”] |
| VAR-B53 | 1.24 | [“Q592I”, “T593V”, “I601A”] |
| VAR-B99 | 1.2 | [“Q579V”, “Q592I”, “V596L”, “N598S”] |
| VAR-B119 | 1.15 | [“Q592I”, “V596L”, “N598S”, “I601A”] |
| VAR-B57 | 1.08 | [“Q579V”, “Q592I”, “V596L”, “N598S”, “I601A”] |
| VAR-B7 | 1.08 | [“Q579V”, “W595A”] |
| VAR-B126 | 1.08 | [“Q592I”, “T593V”, “W595A”, “I601A”] |
| VAR-B71 | 1.07 | [“W595A”, “V596L”, “N598S”, “I601A”] |
| VAR-B3 | 1.06 | [“I601A”] |
| VAR-B6 | 1.06 | [“W595A”] |
| VAR-B32 | 1.05 | [“Q579V”, “Q592I”, “W595A”] |
| VAR-B121 | 1.04 | [“Q592I”, “W595A”, “V596L”, “N598S”] |
| VAR-B58 | 1.04 | [“Q579V”, “Q592I”, “W595A”, “V596L”] |
| VAR-B114 | 1.02 | [“T593V”, “W595A”, “V596L”, “N598S”] |
| VAR-B24 | 0.99 | [“Q592I”, “W595A”] |
| VAR-B98 | 0.98 | [“Q579V”, “V596L”, “I601A”] |
| VAR-B56 | 0.97 | [“T593V”, “W595A”, “N598S”] |
| VAR-B124 | 0.96 | [“Q579V”, “N598S”] |
| VAR-B48 | 0.96 | [“Q579V”, “N598S”, “I601A”] |
| VAR-B9 | 0.95 | [“W595A”, “V596L”] |
| VAR-B79 | 0.94 | [“Q592I”, “V596L”, “N598S”] |
| VAR-B8 | 0.93 | [“Q579V”, “I601A”] |
| VAR-B47 | 0.92 | [“Q592I”, “T593V”, “N598S”] |
| VAR-B96 | 0.88 | [“Q579V”, “T593V”, “V596L”, “N598S”] |
| VAR-B34 | 0.87 | [“Q579V”, “Q592I”, “T593V”, “W595A”] |
| VAR-B45 | 0.83 | [“Q579V”, “T593V”, “N598S”, “I601A”] |
| VAR-B23 | 0.81 | [“Q579V”, “Q592I”, “T593V”] |
| VAR-B75 | 0.79 | [“Q579V”, “T593V”, “W595A”, “V596L”, “N598S”] |
| VAR-B81 | 0.75 | [“Q579V”, “V596L”, “N598S”] |
| VAR-B62 | 0.75 | [“Q579V”, “Q592I”, “I601A”] |
| VAR-B123 | 0.74 | [“Q579V”, “T593V”, “W595A”, “V596L”] |
| VAR-B41 | 0.74 | [“Q579V”, “Q592I”, “W595A”, “N598S”] |
| VAR-B107 | 0.74 | [“Q579V”, “Q592I”, “T593V”, “W595A”, “V596L”] |
| VAR-B88 | 0.73 | [“Q579V”, “T593V”, “V596L”] |
| VAR-B103 | 0.72 | [“W595A”, “N598S”, “I601A”] |
| VAR-B65 | 0.72 | [“Q592I”, “T593V”, “W595A”, “V596L”, “I601A”] |
| VAR-B29 | 0.71 | [“T593V”, “N598S”] |
| VAR-B19 | 0.71 | [“T593V”, “I601A”] |
| VAR-B18 | 0.68 | [“T593V”, “W595A”, “I601A”] |
| VAR-B125 | 0.66 | [“Q592I”, “W595A”, “V596L”, “I601A”] |
| VAR-B33 | 0.63 | [“Q579V”, “T593V”, “W595A”, “N598S”] |
| VAR-B17 | 0.59 | [“T593V”, “W595A”] |
| VAR-B120 | 0.59 | [“Q592I”, “W595A”, “V596L”] |
| VAR-B10 | 0.55 | [“N598S”] |
| VAR-B59 | 0.54 | [“Q592I”, “T593V”, “V596L”, “N598S”] |
| VAR-B109 | 0.54 | [“Q579V”, “W595A”, “N598S”, “I601A”] |
| VAR-B5 | 0.52 | [“Q579V”, “T593V”] |
| VAR-B122 | 0.5 | [“Q579V”, “Q592I”, “T593V”, “W595A”, “V596L”, “N598S”] |
| VAR-B105 | 0.46 | [“Q579V”, “T593V”, “W595A”, “N598S”, “I601A”] |
| VAR-B2 | 0.45 | [“N598S”, “I601A”] |
| VAR-B72 | 0.42 | [“Q579V”, “Q592I”, “T593V”, “V596L”] |
| VAR-B27 | 0.42 | [“Q592I”, “W595A”, “N598S”] |
| VAR-B76 | 0.4 | [“Q579V”, “W595A”, “I601A”] |
| VAR-B101 | 0.39 | [“Q592I”, “V596L”] |
| VAR-B55 | 0.39 | [“T593V”, “W595A”, “V596L”] |
| VAR-B4 | 0.39 | [“T593V”] |
| VAR-B52 | 0.38 | [“Q592I”, “N598S”, “I601A”] |
| VAR-B82 | 0.34 | [“Q579V”, “W595A”, “V596L”, “N598S”] |
| VAR-B44 | 0.34 | [“Q592I”, “N598S”] |
| VAR-B28 | 0.33 | [“Q579V”, “T593V”, “W595A”] |
| VAR-B21 | 0.31 | [“Q579V”, “Q592I”] |
| VAR-B117 | 0.31 | [“Q592I”, “V596L”, “I601A”] |
| VAR-B106 | 0.25 | [“Q579V”, “T593V”, “I601A”] |
| VAR-B70 | 0.23 | [“V596L”, “I601A”] |
| VAR-B37 | 0.23 | [“Q579V”, “Q592I”, “W595A”, “I601A”] |
| VAR-B92 | 0.21 | [“Q579V”, “T593V”, “W595A”, “I601A”] |
| VAR-B63 | 0.2 | [“Q592I”, “W595A”, “V596L”, “N598S”, “I601A”] |
| VAR-B13 | 0.16 | [“Q579V”] |
| VAR-B31 | 0.11 | [“Q579V”, “T593V”, “N598S”] |
| VAR-B22 | 0.08 | [“Q592I”, “I601A”] |
| VAR-B113 | 0.07 | [“Q592I”, “T593V”, “W595A”, “N598S”, “I601A”] |
| VAR-B118 | 0.03 | [“W595A”, “V596L”, “I601A”] |
| VAR-B36 | 0.02 | [“Q579V”, “Q592I”, “T593V”, “N598S”] |
| VAR-B14 | 0.01 | [“V596L”] |
| VAR-B84 | 0.01 | [“Q579V”, “T593V”, “V596L”, “I601A”] |
| VAR-B35 | 0 | [“Q579V”, “Q592I”, “T593V”, “W595A”, “N598S”, “I601A”] |
| VAR-B108 | 0 | [“Q579V”, “Q592I”, “T593V”, “W595A”, “I601A”] |
| VAR-B127 | 0 | [“Q592I”, “W595A”, “I601A”] |
| VAR-B49 | 0 | [“Q579V”, “Q592I”, “T593V”, “I601A”] |
| VAR-B78 | 0 | [“T593V”, “W595A”, “V596L”, “I601A”] |
| VAR-B30 | 0 | [“Q592I”, “T593V”, “N598S”, “I601A”] |
| VAR-B115 | 0 | [“Q579V”, “Q592I”, “V596L”, “I601A”] |
| VAR-B11 | 0 | [“Q592I”, “T593V”] |
| VAR-B64 | 0 | [“Q592I”, “T593V”, “V596L”, “I601A”] |
| VAR-B91 | 0 | [“Q579V”, “Q592I”, “T593V”, “V596L”, “N598S”] |

The skeletal muscle transduction of viral particles containing capsids of VAR-1Bi to VAR-1B127 and VAR-i through VAR-62 (using CBh promoter for the variants VAR-1Bi to VAR-1B127 as set forth in Table 20 and as set forth in Table 29 for VAR-i through VAR-62) was ranked from highest to lowest transduction relative to viral particles containing capsid polypeptides of SEQ ID NO: 1). Results of this analysis are set forth in Table 31.

TABLE 31
Compiled Cbh transduction x mutation data (Var-B1 through Var-B127 and Var1-Var62)

| Var# | x-fold vs AAV9 | Mutations |
|---|---|---|
| VAR-B60 | 5.18 | [“T593V”, “V596L”] |
| VAR-59 | 4.55 | [‘E575W’, ‘H584N’, ‘T593V’, ‘W595A’, ‘V596L’, ‘N598S’, ‘I601V’] |
| VAR-18 | 3.96 | [‘G549T’, ‘Q579T’, ‘T593V’, ‘W595A’, ‘V596L’, ‘N598S’, ‘I601V’] |
| VAR-19 | 3.46 | [‘Q579V’, ‘T582I’, ‘H584N’, ‘T593V’, ‘W595A’, ‘V596L’, ‘N598S’] |
| VAR-58 | 3.45 | [‘Q579S’, ‘A591P’, ‘V596L’, ‘N598S’, ‘I601A’] |
| VAR-42 | 2.94 | [‘Q579T’, ‘H584N’, ‘T593V’, ‘W595A’, ‘V596L’, ‘N598S’] |
| VAR-39 | 2.91 | [‘M559Q’, ‘Q579V’, ‘T582I’, ‘T593V’, ‘W595A’, ‘V596L’, ‘N598S’] |
| VAR-62 | 2.90 | [‘E575S’, ‘H584M’, ‘T593V’, ‘W595A’, ‘V596L’, ‘N598S’] |
| VAR-B61 | 2.89 | [“Q592I”, “T593V”, “V596L”, “N598S”, “I601A”] |
| VAR-45 | 2.88 | [‘Q579T’, ‘Q588N’, ‘A589T’, ‘Q592N’, ‘T593V’, ‘W595A’, ‘V596L’, ‘N598S’, ‘I601V’] |
| VAR-13 | 2.86 | [‘I560Q’, ‘Q579T’, ‘T593V’, ‘W595A’, ‘V596L’, ‘N598S’, ‘I601V’] |
| VAR-51 | 2.82 | [‘Q579T’, ‘Q588N’, ‘A589T’, ‘T593V’, ‘W595A’, ‘V596L’, ‘N598S’, ‘I601V’] |
| VAR-14 | 2.78 | [‘Q579T’, ‘Q588N’, ‘A589T’, ‘Q592S’, ‘T593V’, ‘W595A’, ‘V596L’, ‘N598S’, ‘I601V’] |
| VAR-16 | 2.66 | [‘Q579V’, ‘Q592I’, ‘T593R’, ‘W595A’, ‘V596L’, ‘N598S’] |
| VAR-B54 | 2.65 | [“Q579V”, “T593V”, “W595A”, “V596L”, “N598S”, “I601A”] |
| VAR-50 | 2.64 | [‘Q579V’, ‘A587S’, ‘T593S’, ‘W595A’, ‘V596L’, ‘N598S’, ‘I601V’] |
| VAR-B108 | 2.63 | [“Q579V”, “Q592I”, “T593V”, “W595A”, “I601A”] |
| VAR-B42 | 2.63 | [“Q592I”, “T593V”, “W595A”, “N598S”] |
| VAR-35 | 2.62 | [‘S576W’, ‘Q579V’, ‘A587S’, ‘A589T’, ‘T593V’, ‘W595A’, ‘V596L’, ‘N598S’] |
| VAR-B116 | 2.61 | [“Q579V”, “Q592I”, “W595A”, “V596L”, “N598S”, “I601A”] |
| VAR-5 | 2.53 | [‘M559S’, ‘Q579T’, ‘H584I’, ‘Q592I’, ‘T593V’, ‘W595A’, ‘V596L’, ‘N598S’] |
| VAR-9 | 2.52 | [‘I560L’, ‘Q579V’, ‘A589T’, ‘T593A’, ‘W595A’, ‘V596L’, ‘N598S’] |
| VAR-44 | 2.49 | [‘Q579V’, ‘Q588N’, ‘T593A’, ‘W595A’, ‘V596L’, ‘N598S’, ‘I601V’] |
| VAR-7 | 2.46 | [‘Q579V’, ‘Q588G’, ‘T593S’, ‘W595A’, ‘V596L’, ‘N598S’, ‘I601V’] |
| VAR-20 | 2.46 | [‘G549A’, ‘Q579V’, ‘T593S’, ‘W595A’, ‘V596L’, ‘N598S’, ‘I601V’] |
| VAR-B13 | 2.44 | [“Q579V”] |
| VAR-1 | 2.39 | [‘Q579V’, ‘A589T’, ‘T593A’, ‘W595A’, ‘V596L’, ‘N598S’, ‘I601V’] |
| VAR-23 | 2.37 | [‘T561S’, ‘Q579V’, ‘H584I’, ‘Q592S’, ‘T593V’, ‘W595A’, ‘V596L’, ‘N598S’, ‘I601V’] |
| VAR-38 | 2.35 | [‘G549H’, ‘Q579T’, ‘T593V’, ‘W595A’, ‘V596L’, ‘N598S’, ‘I601V’] |
| VAR-B125 | 2.34 | [“Q592I”, “W595A”, “V596L”, “I601A”] |
| VAR-37 | 2.33 | [‘G549I’, ‘Q579V’, ‘T593S’, ‘W595A’, ‘V596L’, ‘N598S’, ‘I601V’] |
| VAR-40 | 2.22 | [‘Q579V’, ‘T593V’, ‘W595A’, ‘V596L’, ‘N598T’, ‘I601V’] |
| VAR-3 | 2.13 | [‘Q579V’, ‘Q588S’, ‘T593A’, ‘W595A’, ‘V596L’, ‘N598S’, ‘I601V’] |
| VAR-B95 | 2.13 | [“T593V”, “V596L”, “N598S”] |
| VAR-11 | 2.06 | [‘Q579V’, ‘Q588N’, ‘T593A’, ‘W595A’, ‘V596L’,’ N598S’] |
| VAR-B18 | 2.06 | [“T593V”, “W595A”, “I601A”] |
| VAR-43 | 2.05 | [‘Q579V’, ‘Q588N’, ‘T593V’, ‘W595A’, ‘V596L’, ‘N598T’, ‘I601V’] |
| VAR-B99 | 2.04 | [“Q579V”, “Q592I”, “V596L”, “N598S”] |
| VAR-15 | 2.02 | [‘M559Q’, ‘Q579V’, ‘H584Q’, ‘T593V’, ‘W595A’, ‘V596L’, ‘N598S’, ‘I601V’] |
| VAR-55 | 2.02 | [‘Q579V’, ‘H584N’, ‘T593V’, ‘W595A’, ‘V596L’, ‘N598S’, ‘I601V’] |
| VAR-32 | 2.02 | [‘Q579V’, ‘Q588T’, ‘T593A’, ‘W595A’, ‘V596L’, ‘N598S’] |
| VAR-31 | 1.98 | [‘Q579T’, ‘H584L’, ‘T593V’, ‘W595A’, ‘V596L’, ‘N598S’] |
| VAR-B78 | 1.94 | [“T593V”, “W595A”, “V596L”, “I601A”] |
| VAR-25 | 1.94 | [‘T561S’, ‘Q579V’, ‘T593A’, ‘W595A’, ‘V596L’, ‘N598S’, ‘I601V’] |
| VAR-B76 | 1.93 | [“Q579V”, “W595A”, “I601A”] |
| VAR-21 | 1.90 | [‘R550N’, ‘Y577T’, ‘Q579V’, ‘Q592S’, ‘T593V’, ‘W595A’, ‘V596L’, ‘N598S’, ‘I601V’] |
| VAR-28 | 1.88 | [‘V553S’, ‘Q579T’, ‘T593V’, ‘W595A’, ‘V596L’, ‘N598S’, ‘I601V’] |
| VAR-12 | 1.88 | [‘M559V’, ‘Q579V’, ‘T593S’, ‘W595A’, ‘V596L’, ‘N598S’, ‘I601V’] |
| VAR-B20 | 1.84 | [“Q592I”, “W595A”, “N598S”, “I601A”] |
| VAR-B17 | 1.81 | [“T593V”, “W595A”] |
| VAR-B65 | 1.81 | [“Q592I”, “T593V”, “W595A”, “V596L”, “I601A”] |
| VAR-B75 | 1.8 | [“Q579V”, “T593V”, “W595A”, “V596L”, “N598S”] |
| VAR-B80 | 1.79 | [“T593V”, “V596L”, “N598S”, “I601A”] |
| VAR-B106 | 1.78 | [“Q579V”, “T593V”, “I601A”] |
| VAR-B29 | 1.76 | [“T593V”, “N598S”] |
| VAR-B64 | 1.75 | [“Q592I”, “T593V”, “V596L”, “I601A”] |
| VAR-B21 | 1.73 | [“Q579V”, “Q592I”] |
| VAR-6 | 1.72 | [‘M559N’, ‘Q579V’, ‘T593S’, ‘W595A’, ‘V596L’, ‘N598S’, ‘I601V’] |
| VAR-B57 | 1.72 | [“Q579V”, “Q592I”, “V596L”, “N598S”, “I601A”] |
| VAR-24 | 1.66 | [‘Q579V’, ‘H584I’, ‘Q592S’, ‘T593V’, ‘W595A’, ‘V596L’, ‘N598S’, ‘I601V’] |
| VAR-B93 | 1.64 | [“Q592I”, “T593V”, “W595A”, “V596L”, “N598S”] |
| VAR-36 | 1.64 | [‘R550K’, ‘Q579V’, ‘A589S’, ‘T593S’, ‘W595A’, ‘V596L’, ‘N598S’] |
| VAR-4 | 1.61 | [‘G549Y’, ‘Q579T’, ‘T593V’, ‘W595A’, ‘V596L’, ‘N598S’, ‘I601V’] |
| VAR-B117 | 1.6 | [“Q592I”, “V596L”, “I601A”] |
| VAR-B67 | 1.57 | [“Q579V”, “V596L”, “N598S”, “I601A”] |
| VAR-B47 | 1.54 | [“Q592I”, “T593V”, “N598S”] |
| VAR-B100 | 1.5 | [“Q592I”, “T593V”, “W595A”, “V596L”, “N598S”, “I601A”] |
| VAR-B53 | 1.5 | [“Q592I”, “T593V”, “I601A”] |
| VAR-17 | 1.50 | [‘M559C’, ‘Q579V’, ‘T593A’, ‘W595A’, ‘V596L’, ‘N598S’] |
| VAR-22 | 1.49 | [‘R550K’, ‘Q579V’, ‘A589S’, ‘T593A’, ‘W595A’, ‘V596L’, ‘N598S’] |
| VAR-60 | 1.47 | [‘M559A’, ‘Q579T’, ‘S586Q’, ‘A587T’, ‘T593V’, ‘W595A’, ‘V596L’, ‘N598S’] |
| VAR-B23 | 1.46 | [“Q579V”, “Q592I”, “T593V”] |
| VAR-B96 | 1.45 | [“Q579V”, “T593V”, “V596L”, “N598S”] |
| VAR-B70 | 1.45 | [“V596L”, “I601A”] |
| VAR-B14 | 1.44 | [“V596L”] |
| VAR-B118 | 1.44 | [“W595A”, “V596L”, “I601A”] |
| VAR-56 | 1.44 | [‘Q579S’, ‘H584L’, ‘T593V’, ‘W595Y’, ‘V596L’, ‘N598S’, ‘I601V’] |
| VAR-B120 | 1.43 | [“Q592I”, “W595A”, “V596L”] |
| VAR-8 | 1.42 | [‘Y577H’, ‘Q579V’, ‘A581C’, ‘T593V’, ‘W595A’, ‘V596L’, ‘N598S’, ‘I601V’] |
| VAR-48 | 1.39 | [‘Q579T’, ‘S586Q’, ‘A587S’, ‘T593V’, ‘W595A’, ‘V596L’, ‘N598S’, ‘I601V’] |
| VAR-B85 | 1.36 | [“V596L”, “N598S”] |
| VAR-B74 | 1.33 | [“Q579V”, “T593V”, “V596L”, “N598S”, “I601A”] |
| VAR-46 | 1.32 | [‘R550L’, ‘Q579T’, ‘T593V’, ‘W595A’, ‘V596L’, ‘N598S’, ‘I601V’] |
| VAR-27 | 1.31 | [‘Y577T’, ‘Q579V’, ‘Q592S’, ‘T593V’, ‘W595A’, ‘V596L’, ‘N598S’, ‘I601V’] |
| VAR-B87 | 1.3 | [“Q579V”, “W595A”, “V596L”, “N598S”, “I601A”] |
| VAR-10 | 1.29 | [‘M559A’, ‘Q579V’, ‘H584M’, ‘Q592S’, ‘T593V’, ‘W595A’, ‘V596L’, ‘N598S’, ‘I601V’] |
| VAR-B115 | 1.29 | [“Q579V”, “Q592I”, “V596L”, “I601A”] |
| VAR-B9 | 1.29 | [“W595A”, “V596L”] |
| VAR-B86 | 1.28 | [“Q579V”, “V596L”] |
| VAR-B32 | 1.27 | [“Q579V”, “Q592I”, “W595A”] |
| VAR-B8 | 1.26 | [“Q579V”, “I601A”] |
| VAR-B6 | 1.26 | [“W595A”] |
| VAR-B111 | 1.26 | [“Q579V”, “Q592I”, “T593V”, “V596L”, “I601A”] |
| VAR-33 | 1.24 | [‘Y577H’, ‘Q579V’, ‘T582M’, ‘Q592S’, ‘T593V’, ‘W595A’, ‘V596L’, ‘N598S’, ‘I601V’] |
| VAR-B124 | 1.23 | [“Q579V”, “N598S”] |
| VAR-B81 | 1.22 | [“Q579V”, “V596L”, “N598S”] |
| VAR-B2 | 1.22 | [“N598S”, “I601A”] |
| VAR-B121 | 1.22 | [“Q592I”, “W595A”, “V596L”, “N598S”] |
| VAR-B1 | 1.19 | [‘Q579V’, ‘Q592I’, ‘T593V’, ‘W595A’, ‘V596L’, ‘N598S’, ‘I601A’] |
| VAR-B38 | 1.18 | [“Q579V”, “Q592I”, “N598S”, “I601A”] |
| VAR-B52 | 1.18 | [“Q592I”, “N598S”, “I601A”] |
| VAR-B89 | 1.16 | [“T593V”, “W595A”, “V596L”, “N598S”, “I601A”] |
| VAR-2 | 1.13 | [‘D551E’, ‘Q579T’, ‘Q592S’, ‘T593V’, ‘W595A’, ‘V596L’, ‘N598S’, ‘I601V’] |
| VAR-B33 | 1.12 | [“Q579V”, “T593V”, “W595A”, “N598S”] |
| VAR-B48 | 1.11 | [“Q579V”, “N598S”, “I601A”] |
| VAR-B66 | 1.11 | [“Q579V”, “Q592I”, “T593V”, “W595A”, “V596L”, “I601A”] |
| VAR-B68 | 1.1 | [“Q579V”, “W595A”, “V596L”] |
| VAR-B82 | 1.09 | [“Q579V”, “W595A”, “V596L”, “N598S”] |
| VAR-B45 | 1.07 | [“Q579V”, “T593V”, “N598S”, “I601A”] |
| VAR-57 | 1.06 | [‘M559I’, ‘Q579S’, ‘Q592A’, ‘V596L’, ‘N598S’, ‘I601A’] |
| VAR-B25 | 1.05 | [“Q579V”, “Q592I”, “T593V”, “W595A”, “N598S”] |
| VAR-B50 | 1.04 | [“Q579V”, “Q592I”, “N598S”] |
| VAR-B112 | 1.04 | [“W595A”, “V596L”, “N598S”] |
| VAR-B90 | 1.03 | [“Q579V”, “Q592I”, “W595A”, “V596L”, “N598S”] |
| VAR-B58 | 1.03 | [“Q579V”, “Q592I”, “W595A”, “V596L”] |
| VAR-B27 | 1.03 | [“Q592I”, “W595A”, “N598S”] |
| VAR-26 | 1.03 | [‘M559A’, ‘Q579V’, ‘Q592S’, ‘T593V’, ‘W595A’, ‘V596L’, ‘N598T’, ‘I601V’] |
| VAR-34 | 1.01 | [‘G549F’, ‘Q579T’, ‘T593V’, ‘W595A’, ‘V596L’, ‘N598S’, ‘I601V’] |
| VAR-B31 | 1 | [“Q579V”, “T593V”, “N598S”] |
| VAR-B46 | 0.99 | [“Q579V”, “Q592I”, “W595A”, “N598S”, “I601A”] |
| VAR-B19 | 0.99 | [“T593V”, “I601A”] |
| VAR-B59 | 0.97 | [“Q592I”, “T593V”, “V596L”, “N598S”] |
| VAR-B123 | 0.97 | [“Q579V”, “T593V”, “W595A”, “V596L”] |
| VAR-B105 | 0.94 | [“Q579V”, “T593V”, “W595A”, “N598S”, “I601A”] |
| VAR-52 | 0.91 | [‘G549N’, ‘Q579T’, ‘A587S’, ‘A589T’, ‘T593V’, ‘W595A’, ‘V596L’, ‘N598S’] |
| VAR-B10 | 0.86 | [“N598S”] |
| VAR-49 | 0.86 | [‘Q579T’, ‘Q588N’, ‘A589T’, ‘Q592A’, ‘T593V’, ‘W595A’, ‘V596L’, ‘N598S’] |
| VAR-B15 | 0.85 | [“W595A”, “N598S”] |
| VAR-B11 | 0.82 | [“Q592I”, “T593V”] |
| VAR-B119 | 0.81 | [“Q592I”, “V596L”, “N598S”, “I601A”] |
| VAR-B122 | 0.8 | [“Q579V”, “Q592I”, “T593V”, “W595A”, “V596L”, “N598S”] |
| VAR-B7 | 0.8 | [“Q579V”, “W595A”] |
| VAR-B16 | 0.8 | [“W595A”, “I601A”] |
| VAR-B69 | 0.79 | [“Q579V”, “Q592I”, “W595A”, “V596L”, “I601A”] |
| VAR-B36 | 0.79 | [“Q579V”, “Q592I”, “T593V”, “N598S”] |
| VAR-B43 | 0.77 | [“T593V”, “N598S”, “I601A”] |
| VAR-B5 | 0.75 | [“Q579V”, “T593V”] |
| VAR-B114 | 0.74 | [“T593V”, “W595A”, “V596L”, “N598S”] |
| VAR-54 | 0.73 | [‘S576W’, ‘Q579T’, ‘A581N’, ‘T593V’, ‘W595A’, ‘V596L’, ‘N598S’, ‘I601V’] |
| VAR-B101 | 0.71 | [“Q592I”, “V596L”] |
| VAR-B22 | 0.68 | [“Q592I”, “I601A”] |
| VAR-B91 | 0.67 | [“Q579V”, “Q592I”, “T593V”, “V596L”, “N598S”] |
| VAR-B56 | 0.66 | [“T593V”, “W595A”, “N598S”] |
| VAR-B92 | 0.65 | [“Q579V”, “T593V”, “W595A”, “I601A”] |
| VAR-47 | 0.64 | [‘K557N’, ‘Q579V’, ‘Q588N’, ‘T593A’, ‘W595A’, ‘V596L’, ‘N598S’, ‘I601V’, ‘D609N’] |
| VAR-B37 | 0.64 | [“Q579V”, “Q592I”, “W595A”, “I601A”] |
| VAR-61 | 0.63 | [‘M559T’, ‘Q579V’, ‘A587S’, ‘Q588N’, ‘T593S’, ‘W595A’, ‘V596L’, ‘N598S’] |
| VAR-B113 | 0.59 | [“Q592I”, “T593V”, “W595A”, “N598S”, “I601A”] |
| VAR-B83 | 0.56 | [“V596L”, “N598S”, “I601A”] |
| VAR-B71 | 0.56 | [“W595A”, “V596L”, “N598S”, “I601A”] |
| VAR-B98 | 0.55 | [“Q579V”, “V596L”, “I601A”] |
| VAR-B41 | 0.54 | [“Q579V”, “Q592I”, “W595A”, “N598S”] |
| VAR-B3 | 0.54 | [“I601A”] |
| VAR-30 | 0.53 | [‘K557N’, ‘Q579V’, ‘H584L’, ‘A589T’, ‘Q592S’, ‘T593V’, ‘W595A’, ‘V596L’, ‘N598S’, |
| | | ‘I601V’] |
| VAR-B107 | 0.53 | [“Q579V”, “Q592I”, “T593V”, “W595A”, “V596L”] |
| VAR-B12 | 0.53 | [“Q592I”] |
| VAR-B88 | 0.52 | [“Q579V”, “T593V”, “V596L”] |
| VAR-B109 | 0.52 | [“Q579V”, “W595A”, “N598S”, “I601A”] |
| VAR-B51 | 0.51 | [“Q592I”, “T593V”, “W595A”] |
| VAR-B63 | 0.51 | [“Q592I”, “W595A”, “V596L”, “N598S”, “I601A”] |
| VAR-B55 | 0.5 | [“T593V”, “W595A”, “V596L”] |
| VAR-B79 | 0.5 | [“Q592I”, “V596L”, “N598S”] |
| VAR-B40 | 0.46 | [“T593V”, “W595A”, “N598S”, “I601A”] |
| VAR-B39 | 0.44 | [“Q579V”, “Q592I”, “T593V”, “N598S”, “I601A”] |
| VAR-B34 | 0.43 | [“Q579V”, “Q592I”, “T593V”, “W595A”] |
| VAR-B26 | 0.43 | [“Q579V”, “W595A”, “N598S”] |
| VAR-41 | 0.39 | [‘Y577T’, ‘Q579V’, ‘A589S’, ‘Q592A’, ‘T593V’, ‘W595A’, ‘V596L’, ‘N598S’, ‘I601V’] |
| VAR-B44 | 0.35 | [“Q592I”, “N598S”] |
| VAR-B72 | 0.3 | [“Q579V”, “Q592I”, “T593V”, “V596L”] |
| VAR-B62 | 0.28 | [“Q579V”, “Q592I”, “I601A”] |
| VAR-B4 | 0.28 | [“T593V”] |
| VAR-B104 | 0.27 | [“Q592I”, “T593V”, “W595A”, “V596L”] |
| VAR-29 | 0.24 | [‘Q579T’, ‘A587H’, ‘Q588N’, ‘A589T’, ‘Q592I’, ‘T593V’, ‘W595A’, V596L’, ‘N598S’, |
| | | ‘I601V’] |
| VAR-53 | 0.02 | [‘K557L’, ‘M559Q’, ‘Q579V’, ‘T593A’, ‘W595A’, ‘V596L’, ‘N598S’] |
| VAR-B102 | 0 | [“Q579V”, “Q592I”, “T593V”, “V596L”, “N598S”, “I601A”] |
| VAR-B35 | 0 | [“Q579V”, “Q592I”, “T593V”, “W595A”, “N598S”, “I601A”] |
| VAR-B110 | 0 | [“Q592I”, “T593V”, “V596L”] |
| VAR-B127 | 0 | [“Q592I”, “W595A”, “I601A”] |
| VAR-B49 | 0 | [“Q579V”, “Q592I”, “T593V”, “I601A”] |
| VAR-B97 | 0 | [“Q579V”, “W595A”, “V596L”, “I601A”] |
| VAR-B77 | 0 | [“Q579V”, “T593V”, “W595A”, “V596L”, “I601A”] |
| VAR-B28 | 0 | [“Q579V”, “T593V”, “W595A”] |
| VAR-B30 | 0 | [“Q592I”, “T593V”, “N598S”, “I601A” |
| VAR-B103 | 0 | [“W595A”, “N598S”, “I601A”] |
| VAR-B73 | 0 | [“T593V”, “V596L”, “I601A”] |
| VAR-B24 | 0 | [“Q592I”, “W595A”] |
| VAR-B84 | 0 | [“Q579V”, “T593V”, “V596L”, “I601A”] |
| VAR-B94 | 0 | [“Q579V”, “Q592I”, “V596L”] |
| VAR-B126 | 0 | [“Q592I”, “T593V”, “W595A”, “I601A”] |

Without being bound by theory, these data support the finding that inclusion of the variant capsid polypeptides disclosed herein in viral particles results in, for example, increased transduction and expression of a heterologous transgene included in such virus particle, relative to an otherwise similar virus particle comprising the capsid polypeptides of wild-type AAV9 (e.g., the capsid polypeptides of SEQ ID NO:1). These data establish that virus particles comprising a capsid polypeptide comprising any one of the mutations or motifs described herein have unexpectedly marked improvements over otherwise similar virus particles comprising a capsid consisting of wild-type AAV9 capsid polypeptides, including in transduction of a heterologous transgene packaged therein in skeletal muscle cells. Such capsid polypeptides are thus contemplated for use in gene therapies, e.g., virus particles and compositions comprising the same, for use in the treatment of a subject in need thereof, for example, a human, for example for the treatment of a disorder of the skeletal muscle. Suitable disorders of the skeletal muscle that may be treated using such capsid polypeptides, e.g., using virus particles comprising such capsid polypeptides, are disclosed above.

Moreover, as outlined above, the data presented herein establish that virus particles comprising a capsid polypeptide comprising any one of the mutations or motifs described herein have unexpectedly marked improvements over otherwise similar virus particles comprising a capsid consisting of wild-type AAV9 capsid polypeptides, including in transduction of a heterologous transgene packaged therein in cells of the central nervous system (“CNS”), for example, neurons. Accordingly, such capsid polypeptides are contemplated for use in gene therapies, e.g., virus particles and compositions comprising the same, for use in the treatment of a subject in need thereof, for example, a human, for example for the treatment of a disorder of the CNS and the skeletal muscle.

### 7.7. Example 7: In vivo Evaluation of Medium-Throughput Library in Non-Human Primate

### 7.7.1. Methods

### 7.7.1.1. Variant Selection

A library of variants was created utilizing multiple sets of strategies of design and selection. The main objectives were to develop capsids capable of packaging into AAV particles efficiently, transducing central nervous system tissues effectively after intravenous administration, and detargeting the liver and other tissue types. The first set of variants were selected from internal data sets acquired from other non-human primate (NHP) experiments using either: (1) an algorithm that optimizes capsid performance while balancing sequence diversity and measurement uncertainty; (2) the reproducibility of capsid performance across brain regions; or (3) manual curation by examination of individual property performance and sequence diversity. The second set of variants were novel designs that utilized machine learning algorithms trained on extensive data from hundreds of thousands of capsid variants, focusing on optimizing capsid performance further while staying in distribution with respect to the original training set, as well as leveraging methods that exploit additivity observed in the fitness landscape. The study also included variants that contain stop codons in VP1 and VP2 as transduction negative controls (expected to produce virus but not transduce cells) and containing VP3 stop codons as production negative controls (not expected to produce virus). The study also included variants having WT-AAV9 capsid polypeptides as control.

### 7.7.1.2. Library Creation

The virus particles for the study, including those comprising a selection of the variant capsids provided in Table 27 were produced individually via separate transient triple transfection of adherent HEK293T cells followed by co-purification by iodixanol gradient. Each variant capsid was included in a virus particle that included a genome bearing identifying unique barcode sets of 8 as well as diverse random sequence IDs for quantification, providing a measure of biological replicates within the study. Each genome further contains a sequence encoding a fluorescent reporter gene under the control of a ubiquitous Cbh promoter.

The representation of each individual variant within the virus pool is measured via NGS via the unique barcode pools that are associated with each variant. Variants identified with low initial productivity yields were produced again individually in a separate production round and combined with virus from the previous productions to balance the representation of every variant to be within 10-fold range in the final test article. Final test article included each variant at an amount of 9e10-3e11 vg/kg in the IV test article, as measured by ddPCR for final titer and NGS analysis for variant representation. Production efficiencies for individual variants are calculated by NGS relative to production efficiency of wild-type AAV9 in each round of production. For variants that were produced in multiple rounds, production rates were calculated as a mean of production rates across rounds. These rates are provided in Table 33.

### 7.7.1.3. In-vivo Evaluation of Library in NHP

All NHP experiments were conducted in accordance with institutional policies and NIH guidelines. One young adult male and one adult female cynomolgus macaques (Macaca fascicularis) weighing 2.2-2.9 kg, seronegative for anti-AAV9 neutralizing antibodies (NAb) were selected for the study (seronegativity status being serum NAb titers <1:4 based on in vitro NAb assay). Prior to test article administration, samples of blood were collected. The animals were anesthetized with ketamine and xylazine and received an intravenous injection (doses: 1.22e13vg/kg. and 1.27e13vg/kg). During the in-life period the animals were monitored for signs of inflammation and were treated with weekly IM injections of steroids (methylprednisolone, 8 mg/kg), and dexamethasone as needed according to the animal facility's SOPs and recommendations from the veterinarian. Serum samples were collected at 1 h, 12 h and 24 h, 48 h, and weekly after the injections. The animals were sacrificed 4 weeks after the injections and tissues were collected for biodistribution and transduction analyses. The tissues collected are shown in Table 32A. Peripheral tissue samples were collected into RNAlater® (Sigma-Aldrich) and incubated overnight at 4° C., after which the RNAlater®, was drained and samples were frozen at −80° C. The entire brain was sliced into 4 mm coronal slabs using a chilled species-specific brain matrix. Slabs were arranged in sequential order (rostral to caudal). Each slab was bisected into left and right hemispheres along the sagittal midline. The slabs from the right hemisphere were immersed in RNAlater, drained and frozen as previously described. The left hemisphere slabs were sub-dissected for sub regions of the brain, placed into cryo tubes and immediately frozen on dry ice. Brain tissues collected are shown in Table 32B.

TABLE 32A
List of Non-Brain Tissues Collected

Adipose tissue
(abdominal, subcutaneous)
Aorta
Brain
Bone marrow, femur
Carotid artery (left)
Dorsal root ganglion: cervical
(bilateral)
Dorsal root ganglion: thoracic
(bilateral)
Dorsal root ganglion: lumbar
(bilateral)
Dorsal root ganglion: sacral
(bilateral)
Gland, adrenal
Gonad: testes or ovaries
Heart, atria (right)
Heart, apex
Heart, ventricle (left)
Kidney
Liver, right lateral lobe
Liver, left lateral lobe
Lung, cranial lobe (bilateral)
Lymph nodes, cervical
Nerve, sciatic (right)
Skeletal muscle, biceps brachii (bilateral)
Skeletal muscle, diaphragm
Skeletal muscle, gastrocnemius
Skeletal muscle, quadriceps (bilateral)
Spinal cord, cervical
Spinal cord, cranial thoracic
Spinal cord, caudal thoracic
Spinal cord, lumbar
Spleen

TABLE 32B
List of Brain Tissues Collected

Basal ganglia (caudate, globus pallidus, putamen)
Brainstem (medulla, pons)
Cerebellum
Forebrain (frontal cortex, motor cortex)
Hippocampus
Midbrain
Parietal cortex
Substantia nigra
Temporal cortex
Thalamus

### 7.7.1.4. Bulk Sequencing NGS Library Preparation

Brain slices were dissected to isolate regions including, but not limited to, frontal cortex, temporal cortex, motor cortex, hippocampus, basal ganglia, midbrain, brainstem, and cerebellum. For all biodistribution and transduction analyses, total DNA and RNA was extracted from tissue samples with Trizol/chloroform using DNeasy Blood & Tissue kit (Qiagen) and RNeasy kit respectively. RNA was extracted from the upper aqueous phase using a RNeasy (QIAGEN) kit according to the manufacturer's recommendations, and was further treated with Turbo™ DNase (Thermo Fisher Scientific) to remove any vector DNA contamination in the RNA sample. Reverse transcription was done with Protoscript II Reverse Transcriptase (New England Biolabs) utilizing primers that were specific to the vector transgene and included unique molecular identifiers (UMIs). Control reactions lacking the reverse transcriptase enzyme (-RT control) were also prepared. Finally, samples were prepared for next-generation sequencing by amplifying the transgene barcode regions with primers compatible with Illumina NGS platform and sequenced with NextSeq 550 (Illumina).

### 7.7.1.5. Bulk Tissue NGS Sample Parsing and Analysis

After sequencing, the barcode tags were extracted from reads with the expected amplicon structure, and the abundance (number of reads, number of UMIs, or number of unique id tags) of each barcode was recorded. Analyses were restricted to the set of barcodes that were present in the input virus sample, as measured by a separate sequencing assay of the input virus sample. To aggregate biodistribution samples, read counts from samples from the same tissue were summed. To aggregate brain transduction samples, the number of transduction events (measured by unique id tags detected) from samples from the same tissue were summed. To aggregate all other transduction samples, the number of UMIs from samples from the same tissue were summed.

Biodistribution and transduction of tissue were calculated by normalizing aggregated biodistribution or transduction counts with input virus abundance. The transduction and biodistribution rates were calculated as fold change relative to the wild-type (WT) AAV9. The measurements are reported as mean and standard deviation of barcode replicates (n=8-16).

### 7.7.1.6. Single-nuclei RNA Sequencing and Analysis

Single-cell RNA sequencing has been previously demonstrated to allow characterization of cell-type specific tropism of barcoded rAAVs (Brown et al., 2021, Front. Immunol. 12:730825, which is incorporated herein by reference in its entirety). However, obtaining single cell suspension from certain tissue types and/or flash frozen samples from externally-sourced NHP studies can be extremely challenging. An approach was developed that combines single-nuclei RNA sequencing (snRNA-Seq) with targeted amplicon sequencing to reliably detect cell-type specific transduction from up to 50-100 barcoded rAAVs with minimal sequencing depth, with an initial focus on tissues of the eye, and applied these methods to brain tissues collected from the experiment described in this example. To implement this approach: 1) protocols for isolation of high quality single nuclei suspensions from flash frozen NHP brain tissues (e.g. prefrontal cortex, motor cortex, caudate, putamen) were developed, 2) the 10× Genomics Chromium X platform was used to encapsulate these nuclei and generate gene expression libraries for reliable identification of cell types, 3) barcoded viral transcripts that were captured using the 10× oligo dT capture probes were selectively amplified (for sequencing), and 4) viral transcripts identified from the targeted libraries were computationally mapped to cells identified using the gene expression libraries. Using this approach, cell-type specific tropism of multiple rAAVs in Cynomolgus macaque cortex (prefrontal+motor cortex), and basal ganglia regions (caudate+putamen) were investigated. The snRNA-seq gene expression analysis focused on therapeutically relevant neuron cell populations. Overall, it was demonstrated that relative transduction between multiple rAAVs in neuronal cells can be quantitated. Details of the single nuclear experimental workflow that was applied to the medium throughput study described in this example are below:

### 7.7.1.6.1. Materials

Materials included the following:
EZ lysis buffer+0.2 U/μl of murine RNAse inhibitor
1×PBS+20% BSA+0.2U/μl of murine RNase inhibitor
1×PBS+5% BSA+0.2 U/μl of murine RNAse inhibitor
1×PBS+2% BSA+0.2 U/μl of murine RNAse inhibitor
Eight 15 ml tubes pre-coated with 1×PBS+2% BSA+0.2 U/μl RNAse inhibitor
Two 50 ml tubes pre-coated with 1×PBS+2% BSA+0.2 U/μl RNAse inhibitor
Four polypropylene FACS tubes pre-coated with 1×PBS+2% BSA+0.2 U/μl RNAse inhibitor, one with 100 uL 1×PBS+5% BSA+0.2 U/μl RNAse inhibitor in the bottom for collection
Four 7 ml dounce homogenizer+Pestle A and Pestle B
2×Dissection Scissors
2×0.4 pm filters
2×0.7 pm filters
1 ml wide bore pipette tips

### Hemocytometer

Six 0.5 ml protein lobind tube with 15 μl of 1×PBS+5% BSA+0.2U/μl murine RNase inhibitor+1:100 Propidium Iodide
Two 0.5 ml protein lobind tubes with 1 uL Propidium Iodide
Sony MA900 Cell Sorter

### 7.7.1.6.2. Single nuclei dissociation from Prefrontal Cortex, Motor Cortex, Caudate and Putamen

Mincing: All CNS tissue pieces were cut into four ~25 mg pieces on a cryostat and weighed. The 25 mg tissue samples were placed in a tube on ice and 200 μl of EZ lysis buffer+RNAse inhibitor was added. The tissue was minced with a pair of microscissors for about 1 min and transferred to a 7 ml dounce homogenizer using a wide bore pipette. Each ~25 mg tissue piece was homogenized in a separate 7 ml dounce.

Dounce homogenization: EZ lysis buffer+RNAse inhibitor was added to the sample in the dounce homogenizer for a total volume of 7 ml. A loose-fitting pestle (Pestle A) was used to dounce the sample with steady strokes (about 1 stroke per second) followed by a tight-fitting pestle (Pestle B). The number of dounces varies by tissue type and is indicated in the table below.

| Tissue | Dounce A | Dounce B |
|---|---|---|
| Frontal Cortex | 10x | 5x, wait 20 seconds on ice, 5x |
| Motor Cortex | 10x | 5x, wait 20 seconds on ice, 5x |
| Caudate | 10x | 5x, wait 20 seconds on ice, 5x |
| Putamen | 10x | 5x, wait 20 seconds on ice, 5x |

Filtration and clean up: Post dounce, a 70 pm filter was stacked on top of a 40 pm filter over a 50 mL conical centrifuge tube pre-coated with 1×PBS+2% BSA+0.2 U/μl of murine RNAse inhibitor. Using a funnel, the sample (7 ml) was passed through the filters by pouring. Next, 7 ml of 1×PBS+20% BSA+0.2 U/μl of murine RNAse inhibitor was passed through the funnel and filters to rinse. Mincing, douncing and filtration was repeated with a second 25 mg tissue piece and both pieces of tissue were combined during the filtering step. A small (10 pl) aliquot of the filtered sample was transferred to a 0.5 ml protein lobind tube with 1ul Propidium Iodide for counting. The filtered sample was divided into 4 pre-coated 15 ml conical centrifuge tubes with 7 ml sample each. The sample tubes were centrifuged at 200 RCF for 10 mins at 4° C. The supernatant was discarded and the pellet was resuspended in appropriate volume of 1×PBS+5% BSA+RNAse-Inhibitor to obtain ~3 million nuclei/ml. A small (5 pl) aliquot of the sample was transferred to a 0.5 ml protein lobind tube with 15 μl of 1×PBS+5% BSA+0.2U/μl murine RNase inhibitor+1:100 Propidium Iodide for counting. The remaining sample was stained with 1:100 Draq7.

FACS cleanup: The nuclei were sorted on a Sony MA900 cell sorter by gating for intact nuclei that were positively stained for Draq7 and discarding any doublets. The FACS cleaned nuclei were centrifuged at 200 RCF for 10 mins at 4° C. in a final concentration of 10% BSA. The pellet was resuspended in 1×PBS+2% BSA+RNAse-inhibitor and counted. Final nuclei concentration was adjusted as needed for 10× encapsulation.

10× Encapsulation and library preparation: The 10× Chromium X platform (10× Genomics) was used for single cell encapsulation as per the manufacturer's standard instructions. Reverse transcription was performed as per 10× protocols. cDNA amplification was performed using the 10×cDNA amplification kit which allows for amplification of oligo-dT captured transcripts. A viral transcript specific primer was added during cDNA amplification to enable early amplification and purification of viral transcripts. Post cDNA amplification, a portion of the cDNA library was used to generate a gene expression library as per 10× standard protocol and the library was quality controlled and sequenced as per standard 10× protocols. A small portion of the cDNA library was used to generate targeted libraries by PCR amplifying the Dyno barcode region. For targeted amplification of viral transcripts captured by oligo-dT, primers binding to the TruSeq Handle in combination with a viral transcript specific primer were used. Once the targeted product was amplified we performed pre-indexing and indexing PCRs and sequenced the libraries using an Illumina NextSeq2000 sequencer.

### 7.7.1.6.3. Single Nuclei RNA Sequencing Data Analysis

Gene expression data processing: 10× gene expression libraries were sequenced at a depth of 5000 reads per nuclei. Gene expression sequencing data was demultiplexed using Illumina bcl-convert with default settings, then aligned to Macaca fascicularis reference genome (v6.0, assembly GCA_011100615.1) and quantified using the CellRanger pipeline v7.1.0 with intron mode activated. Doublet detection and filtering was performed using Scrublet package v0.2.3. Dimensionality reduction, batch effect removal, clustering, and identification of marker genes were carried out using Scanpy v1.9.3.

Cell type annotation: 10× gene expression RNA transcript data was plotted on a UMAP plot (Leiden clustering) to reveal the cell clusters. Identification of cell types was performed with an in-house algorithm that projects cell type labels from reference datasets, which were curated from published literature (Siletti et al., 2023, Science, 382(6667):eadd7046, which is incorporated herein by reference in its entirety). This allowed us to annotate major cell types in the CNS tissues, including neurons, oligodendrocytes, oligodendrocyte precursors, astrocytes, microglia, and vascular cells. To validate the annotations, curated cell type specific markers from the literature were used (Khrameeva et al., 2020, Genome Res. 30(5):776-789; Han et al., 2022, Nature 604(7907):723-731; He et al., 2021, Current Biology 31(24):5473-5486.e6; Agarwal et al., 2020, Nature Comms. 11:4183, each of which is incorporated herein by reference in its entirety) and it was confirmed that these markers follow the expected expression patterns in the data.

Targeted library data processing: Targeted libraries were processed using an in-house pipeline to obtain the identities of transducing variants and the 1OX feature barcode. To completely remove chimeric molecules, transcript per transcript (TPT) filtering (Dixit, 2021, bioRxiv 093237, which is incorporated herein by reference in its entirety) was performed with a threshold of 0.5 and 0.02 for forward and reverse molecules, respectively. Targeted libraries were then filtered against gene expression libraries to associate cell type information and limit the analysis to valid cell barcodes. The data was further filtered with cut-offs of 250-1000 reads per molecule to remove any remaining sequencing artifacts. Finally, nuclei with more than 3 observed transduction events, which likely represent clumping artifacts, were excluded from downstream analysis.

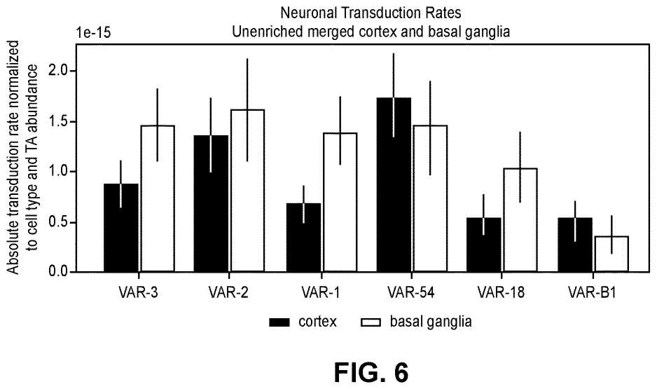

Determination of transduction rates: To calculate the normalized transduction rate of variant ‘i’ in cell type ‘j’, the number of transduction events for variant ‘i’ observed in cell type ‘j’ was divided by the population count of cell type ‘j’. To generate the data shown in FIG. 6, this value was further normalized by the amount of vector genome (vg) dosed, which is defined as the fraction of reads (RNAseq) belonging to variant ‘i’ in the test article, multiplied by total vg dosed into the brain [transduction efficiency of variant ‘i’ in cell type ‘j’=(transduction events ‘i’/number of cells ‘j’)/#of dose vector genomes for variant ‘i’]. The error bars were computed by bootstrapping cell barcodes (N=2000), calculating the resulting resampled rates, and identifying the 5th and 95th percentiles of the sampled distributions.

### 7.7.2. Results

The production, biodistribution and transduction results for the selection of variants are summarized in Table 33 (production), Table 34 (spinal cord and periphery biodistribution), Tables 35-1 and 35-2 (CNS transduction) and Table 36 (spinal cord and periphery transduction).

TABLE 33
Production efficiency of variant virus particles relative to production
efficiency of WT AAV9 virus particles, in HEK293 cells in vitro.

| VAR-# | Fold Change Relative to wtAAV9 |
|---|---|
| VAR-29 | 0.54 |
| VAR-9 | 0.71 |
| VAR-55 | 0.84 |
| VAR-60 | 0.61 |
| VAR-57 | 0.45 |
| VAR-61 | 0.6 |
| VAR-54 | 0.95 |
| VAR-16 | 0.84 |
| VAR-2 | 0.76 |
| VAR-18 | 1.28 |
| VAR-33 | 0.65 |
| VAR-26 | 1.15 |
| VAR-5 | 0.95 |
| VAR-1 | 1.62 |
| VAR-3 | 1.54 |

Almost all 15 variant capsids evaluated in this assessment increased biodistribution in all skeletal muscle tissue samples and the spinal cord, but not in other non-brain tissues, as compared to capsid polypeptides of SEQ ID NO:1. Results of this analysis are summarized in Table 34.

All 15 variant capsids evaluated in this assessment increased transduction in all brain samples as compared to capsid polypeptides of SEQ ID NO:1. Results of this analysis are summarized in Tables 35-1 and 35-2. Similar transduction increases were observed with spinal cord samples as well (Table 36). Variant capsids evaluated in this assessment also increased transduction in muscle samples, even though these transduction increases were not as large as the transduction increases in brain and spinal cord samples (Table 36).

FIG. 6 shows neuronal transduction rates of VAR-1, VAR-2, VAR-3, VAR-18, VAR-54, and VAR-B1 for cortex and basal ganglia tissue samples. As shown in FIG. 6, VAR-1, VAR-2, VAR-3, VAR-18, and VAR-54 each have improved neuronal transduction relative to VAR-B1 in the cortex and/or basal ganglia.

Without being bound by theory, these data support the finding that inclusion of the variant capsid polypeptides disclosed herein in viral particles results in, for example, increased transduction and expression of a heterologous transgene included in such virus particle, relative to an otherwise similar virus particle comprising the capsid polypeptides of wild-type AAV9 (e.g., the capsid polypeptides of SEQ ID NO:1). These data establish that virus particles comprising a capsid polypeptide comprising any one of the mutations or motifs described herein have unexpectedly marked improvements over otherwise similar virus particles comprising a capsid consisting of wild-type AAV9 capsid polypeptides, including in transduction of a heterologous transgene packaged therein in skeletal muscle cells and in cells of the central nervous system (“CONS”), for example, cells in the spinal cord and brain. Such capsid polypeptides are contemplated for use in gene therapies, e.g., virus particles and compositions comprising the same, for use in the treatment of a subject in need thereof, for example, a human, for example for the treatment of a disorder of the CNS and the skeletal muscle.

TABLE 34
Fold change in biodistribution of virus particles comprising the indicated variant capsid polypeptide, relative to the biodistribution
of virus particles comprising WT AAV9 for non-brain tissue, with the standard deviation shown in parentheses following each
value. “Aggregated” in relation to a tissue is an average across all measured areas in the specified organ.

| | Organ | | | | | | | |
|---|---|---|---|---|---|---|---|---|
| | Dorsal | | | | | | | |
| | Root | | Skeletal | Skeletal | Skeletal | Skeletal | | |
| | Ganglia | Liver | Muscle | Muscle | Muscle | Muscle | Spinal Cord | Spleen |
| | Tissue | | | | | | | |
| Variant # | aggregated | aggregated | aggregated | biceps | gastrocnemius | quadriceps | aggregated | aggregated |
| VAR-29 | 1.49 (1.18) | 0.26 (1.05) | 2.07 (1.09) | 1.91 (1.11) | 2.37 (1.16) | 1.89 (1.07) | 5.23 (1.78) | 0.49 (1.09) |
| VAR-9 | 0.99 (1.49) | 0.46 (1.08) | 0.96 (1.21) | 0.83 (1.62) | 1.15 (1.18) | 0.81 (1.35) | 3.31 (2.98) | 0.57 (1.12) |
| VAR-55 | 1.23 (1.38) | 0.75 (1.13) | 2 (1.16) | 1.78 (1.23) | 2.19 (1.44) | 1.85 (1.22) | 2.94 (2.14) | 0.95 (1.15) |
| VAR-60 | 0.69 (1.67) | 0.28 (1.1) | 0.62 (1.29) | 0.71 (1.51) | 0.63 (1.58) | 0.46 (1.8) | 0.58 (2.91) | 0.49 (1.1) |
| VAR-57 | 0.97 (1.47) | 0.25 (1.05) | 1.15 (1.14) | 1.12 (1.37) | 1.13 (1.22) | 1.15 (1.19) | 2.06 (2.87) | 0.59 (1.11) |
| VAR-61 | 0.31 (1.62) | 0.12 (1.11) | 0.31 (1.34) | 0.31 (1.62) | 0.3 (1.35) | 0.28 (2.11) | 0.51 (6.34) | 0.35 (1.11) |
| VAR-54 | 1.53 (1.16) | 0.12 (1.1) | 3.86 (1.11) | 3.66 (1.18) | 4.33 (1.2) | 3.49 (1.08) | 8.42 (1.65) | 0.2 (1.09) |
| VAR-16 | 0.79 (1.37) | 0.17 (1.11) | 1.76 (1.19) | 1.73 (1.37) | 2.3 (1.17) | 1.2 (1.31) | 1.56 (1.5) | 0.17 (1.25) |
| VAR-2 | 1.53 (1.25) | 0.35 (1.05) | 1.81 (1.16) | 1.61 (1.27) | 2.07 (1.24) | 1.63 (1.26) | 6.04 (1.61) | 0.93 (1.07) |
| VAR-18 | 1.36 (1.37) | 0.35 (1.08) | 2.24 (1.04) | 1.92 (1.21) | 2.84 (1.15) | 1.77 (1.33) | 5.98 (2.72) | 0.73 (1.12) |
| VAR-33 | 0.83 (1.4) | 0.18 (1.05) | 1.08 (1.16) | 1.09 (1.64) | 1.07 (1.27) | 1 (1.24) | 3.34 (1.93) | 0.3 (1.12) |
| VAR-26 | 0.85 (1.59) | 0.22 (1.11) | 1.03 (1.17) | 0.88 (1.21) | 1.21 (1.3) | 0.94 (1.12) | 1.65 (1.65) | 0.65 (1.08) |
| VAR-5 | 0.84 (1.78) | 0.14 (1.08) | 1.07 (1.16) | 0.91 (1.42) | 1.23 (1.26) | 0.97 (1.29) | 1.58 (2.01) | 0.63 (1.13) |
| VAR-1 | 1.36 (1.26) | 0.38 (1.07) | 2.43 (1.1) | 2.29 (1.23) | 2.75 (1.2) | 2.14 (1.14) | 5.22 (1.7) | 0.66 (1.08) |
| VAR-3 | 1.56 (1.23) | 0.43 (1.05) | 2.95 (1.13) | 2.76 (1.22) | 3.57 (1.15) | 2.43 (1.18) | 7.05 (1.5) | 0.58 (1.07) |

TABLE 35
1. Fold change in transduction of virus particles comprising the indicated variant capsid polypeptide, relative to the biodistribution
of virus particles comprising WT AAV9 for brain tissue, with the standard deviation shown in parentheses following each
value. “Aggregated” in relation to a tissue is an average across all measured areas in the specified organ.

| | Organ | | | | | | | |
|---|---|---|---|---|---|---|---|---|
| | Brain | Brain | Brain | Brain | Brain | Brain | Brain | Brain |
| | Region | | | | | | | |
| | | Basal | Basal | Basal | Basal | | | |
| | aggregated | Ganglia | Ganglia | Ganglia | Ganglia | Brainstem | Cerebellum | Forebrain |
| | Tissue | | | | | | | |
| | | | | Globus | | | | |
| Variant # | aggregated | aggregated | Caudate | Pallidus | Putamen | aggregated | aggregated | aggregated |
| VAR-29 | 41.84 (1.21) | 40.87 (1.18) | 38.03 (1.14) | 34.54 (1.24) | 49.36 (1.22) | 27.32 (1.2) | 56.5 (1.14) | 47.77 (1.28) |
| VAR-9 | 26.87 (1.22) | 22.68 (1.18) | 23.12 (1.22) | 19.76 (1.31) | 22.8 (1.32) | 18.51 (1.35) | 33.51 (1.27) | 32.37 (1.21) |
| VAR-55 | 47.18 (1.15) | 42.18 (1.13) | 40.6 (1.1) | 39.75 (1.38) | 45.29 (1.21) | 35.55 (1.23) | 66.33 (1.16) | 47.81 (1.14) |
| VAR-60 | 8.37 (1.09) | 7.75 (1.14) | 8.09 (1.16) | 2.87 (8.44) | 8.46 (1.24) | 5.98 (1.15) | 10.58 (1.2) | 8.86 (1.19) |
| VAR-57 | 19.04 (1.3) | 18.11 (1.29) | 16.87 (1.3) | 17.77 (1.34) | 20.26 (1.29) | 12.06 (1.32) | 26.69 (1.32) | 21.6 (1.35) |
| VAR-61 | 6.07 (1.13) | 5.81 (1.2) | 5.73 (1.3) | 5.32 (1.72) | 5.66 (1.34) | 4.11 (1.23) | 6.56 (1.17) | 7.21 (1.19) |
| VAR-54 | 58.24 (1.07) | 55.8 (1.06) | 51.35 (1.06) | 47.7 (1.1) | 68.05 (1.07) | 37.75 (1.08) | 73.8 (1.06) | 70.85 (1.09) |
| VAR-16 | 27.63 (1.11) | 22.51 (1.09) | 18.72 (1.16) | 20.16 (1.14) | 29.95 (1.09) | 13.52 (1.07) | 36.76 (1.11) | 40.57 (1.18) |
| VAR-2 | 69.78 (1.06) | 67.61 (1.04) | 61.52 (1.06) | 60.16 (1.07) | 82.07 (1.08) | 40.3 (1.12) | 84.38 (1.09) | 86.11 (1.08) |
| VAR-18 | 54.58 (1.08) | 48.77 (1.07) | 44.96 (1.12) | 44.38 (1.14) | 57.32 (1.09) | 35.21 (1.13) | 73.26 (1.09) | 64.21 (1.1) |
| VAR-33 | 22.76 (1.11) | 26.25 (1.15) | 24.45 (1.19) | 23.03 (1.2) | 30.85 (1.18) | 13.27 (1.18) | 25.63 (1.14) | 26.24 (1.14) |
| VAR-26 | 15.45 (1.08) | 17.25 (1.08) | 16.28 (1.15) | 14.23 (1.16) | 20.39 (1.15) | 9.49 (1.11) | 18.7 (1.13) | 17.26 (1.13) |
| VAR-5 | 22.89 (1.14) | 21.43 (1.18) | 19.85 (1.25) | 16.26 (1.34) | 26.69 (1.17) | 12.73 (1.19) | 26.91 (1.14) | 29.67 (1.17) |
| VAR-1 | 67.93 (1.05) | 61.06 (1.04) | 56.63 (1.05) | 55.49 (1.08) | 71.65 (1.07) | 45.48 (1.08) | 86.56 (1.05) | 78.26 (1.06) |
| VAR-3 | 78.58 (1.07) | 70.39 (1.06) | 65.83 (1.07) | 65.67 (1.08) | 80.77 (1.05) | 55.91 (1.07) | 105.09 (1.06) | 86.87 (1.08) |

2. Fold change in transduction of virus particles comprising the indicated variant capsid polypeptide, relative to the biodistribution
of virus particles comprising WT AAV9 for brain tissue, with the standard deviation shown in parentheses following each
value. “Aggregated” in relation to a tissue is an average across all measured areas in the specific organ.

| | Organ | | | | | | | |
|---|---|---|---|---|---|---|---|---|
| | Brain | Brain | Brain | Brain | Brain | Brain | Brain | Brain |
| | Region | | | | | | | |
| | | | | | Parietal | Substantia | Temporal | |
| | Forebrain | Forebrain | Hippocampus | Midbrain | Cortex | Nigra | Cortex | Thalamus |
| | Tissue | | | | | | | |
| | Frontal | Motor | | | | | | |
| Variant # | Cortex | Cortex | Aggregated | Aggregated | Aggregated | Aggregated | Aggregated | Aggregated |
| VAR-29 | 42.2 (1.31) | 49.51 (1.28) | 62.46 (1.27) | 35.19 (1.2) | 53.75 (1.27) | 22.1 (1.17) | 83.67 (1.31) | 62.04 (1.24) |
| VAR-9 | 26.3 (1.44) | 34.09 (1.2) | 36.26 (1.45) | 23.9 (1.19) | 34 (1.22) | 15.03 (1.33) | 51.46 (1.18) | 43.2 (1.22) |
| VAR-55 | 40.22 (1.18) | 50.13 (1.15) | 67.55 (1.2) | 43.23 (1.2) | 58.28 (1.2) | 30.67 (1.2) | 86.04 (1.18) | 65.34 (1.15) |
| VAR-60 | 6.53 (1.14) | 9.52 (1.27) | 10.42 (1.46) | 6.84 (1.31) | 10.69 (1.53) | 5.21 (1.21) | 16.53 (1.34) | 13.52 (1.21) |
| VAR-57 | 17.73 (1.56) | 22.68 (1.32) | 21.1 (1.33) | 15.9 (1.32) | 23.38 (1.4) | 10.5 (1.36) | 32.37 (1.37) | 33.7 (1.43) |
| VAR-61 | 6.36 (1.27) | 7.43 (1.23) | 6.56 (1.39) | 5.16 (1.2) | 9.01 (1.23) | 3.51 (1.34) | 10.02 (1.53) | 9.91 (1.31) |
| VAR-54 | 75.85 (1.08) | 69.17 (1.1) | 107.78 (1.09) | 46.34 (1.09) | 80.27 (1.1) | 27.07 (1.12) | 143.8 (1.11) | 74.71 (1.1) |
| VAR-16 | 28.83 (1.2) | 44.35 (1.19) | 50.33 (1.14) | 24.63 (1.09) | 38.92 (1.17) | 10.97 (1.44) | 54.51 (1.14) | 38.58 (1.16) |
| VAR-2 | 70.96 (1.17) | 90.87 (1.07) | 105.48 (1.12) | 57.05 (1.07) | 100.26 (1.13) | 32.99 (1.15) | 148.8 (1.07) | 119.13 (1.06) |
| VAR-18 | 54.68 (1.16) | 67.23 (1.1) | 82.36 (1.09) | 46.57 (1.08) | 71.33 (1.07) | 29.15 (1.11) | 108.53 (1.1) | 87.64 (1.16) |
| VAR-33 | 21.48 (1.4) | 27.55 (1.13) | 30.07 (1.23) | 18.84 (1.17) | 28.12 (1.29) | 12.7 (1.2) | 39.32 (1.31) | 38.66 (1.16) |
| VAR-26 | 18.94 (1.16) | 16.66 (1.16) | 28.2 (1.17) | 12.12 (1.1) | 18.53 (1.15) | 7.26 (1.13) | 40.68 (1.06) | 21.73 (1.13) |
| VAR-5 | 22.24 (1.25) | 32.03 (1.17) | 34.7 (1.15) | 17.38 (1.11) | 29.6 (1.2) | 11.21 (1.23) | 46.15 (1.34) | 45.84 (1.18) |
| VAR-1 | 71.65 (1.08) | 80.38 (1.06) | 114.43 (1.1) | 59.96 (1.06) | 93.37 (1.05) | 36.93 (1.11) | 146.15 (1.11) | 95.97 (1.05) |
| VAR-3 | 81.35 (1.1) | 88.62 (1.08) | 129.05 (1.07) | 68.82 (1.08) | 103.82 (1.07) | 44.12 (1.13) | 165.44 (1.12) | 108.3 (1.11) |

TABLE 36
Fold change in transduction of virus particles comprising the indicated variant capsid polypeptide, relative to the biodistribution
of virus particles comprising WT AAV9 for non-brain tissue, with the standard deviation shown in parentheses following each
value. “Aggregated” in relation to a tissue is an average across all measured areas in the specified organ.

| | Organ | | | | | | | |
|---|---|---|---|---|---|---|---|---|
| | Dorsal Root | | Skeletal | Skeletal | Skeletal | Skeletal | | |
| | Ganglia | Liver | Muscle | Muscle | Muscle | Muscle | Spinal Cord | Spleen |
| | Tissue | | | | | | | |
| Variant # | aggregated | aggregated | aggregated | Biceps | Gastrocnemius | Quadriceps | aggregated | aggregated |
| VAR-29 | 2.78 (1.33) | 0.25 (1.12) | 5.22 (1.07) | 4.83 (1.07) | 5.33 (1.08) | 5.36 (1.07) | 37.09 (1.59) | 0.48 (1.3) |
| VAR-9 | 1.94 (1.49) | 0.45 (1.06) | 2.08 (1.43) | 1.97 (1.4) | 2.1 (1.45) | 2.16 (1.42) | 18.98 (1.32) | 0.56 (1.48) |
| VAR-55 | 2.38 (1.43) | 0.7 (1.22) | 5.07 (1.15) | 4.69 (1.18) | 5.15 (1.14) | 5.28 (1.16) | 33.99 (1.38) | 1.14 (1.42) |
| VAR-60 | 1.13 (1.33) | 0.28 (1.29) | 0.97 (1.11) | 0.96 (1.14) | 0.98 (1.09) | 0.97 (1.18) | 4.41 (1.74) | 0.4 (1.45) |
| VAR-57 | 1.65 (1.44) | 0.28 (1.04) | 2.08 (1.16) | 1.98 (1.18) | 2.1 (1.15) | 2.16 (1.19) | 12.72 (1.32) | 0.44 (1.27) |
| VAR-61 | 0.49 (2.08) | 0.1 (1.11) | 0.44 (1.15) | 0.38 (1.18) | 0.43 (1.17) | 0.52 (1.18) | 3.15 (1.72) | 0.39 (1.54) |
| VAR-54 | 1.56 (1.73) | 0.08 (1.23) | 8.59 (1.16) | 8.02 (1.14) | 8.7 (1.17) | 8.93 (1.16) | 72.17 (1.16) | 0.22 (1.87) |
| VAR-16 | 0.75 (1.36) | 0.15 (1.05) | 7.66 (1.06) | 7.34 (1.06) | 8.18 (1.06) | 6.8 (1.06) | 25.42 (1.32) | 0.19 (1.4) |
| VAR-2 | 3.43 (1.24) | 0.36 (1.08) | 5.11 (1.08) | 4.83 (1.08) | 5.28 (1.08) | 5.01 (1.08) | 51.94 (1.14) | 0.86 (1.27) |
| VAR-18 | 1.81 (1.2) | 0.34 (1.09) | 4.91 (1.11) | 4.66 (1.11) | 5.1 (1.12) | 4.75 (1.11) | 45.38 (1.19) | 0.81 (1.38) |
| VAR-33 | 0.43 (1.99) | 0.18 (1.08) | 0.94 (1.13) | 0.91 (1.14) | 0.99 (1.14) | 0.85 (1.14) | 16.39 (1.42) | 0.28 (1.87) |
| VAR-26 | 1 (1.2) | 0.22 (1.05) | 1.3 (1.08) | 1.21 (1.08) | 1.35 (1.09) | 1.26 (1.12) | 12.69 (1.21) | 0.5 (1.28) |
| VAR-5 | 1.17 (1.8) | 0.15 (1.07) | 1.47 (1.12) | 1.36 (1.15) | 1.51 (1.13) | 1.51 (1.14) | 16.02 (1.26) | 0.36 (1.68) |
| VAR-1 | 2.3 (1.24) | 0.36 (1.06) | 6.13 (1.11) | 5.54 (1.11) | 6.33 (1.12) | 6.29 (1.11) | 60.08 (1.2) | 0.63 (1.28) |
| VAR-3 | 2.45 (1.37) | 0.41 (1.1) | 7.76 (1.12) | 7.02 (1.11) | 7.98 (1.12) | 8.04 (1.15) | 74.11 (1.21) | 0.78 (1.28) |

### 8. SEQUENCE LISTING

Exemplary sequences of the present disclosure are provided in Table 37 below, where SEQ=SEQ ID NO.

TABLE 37

| Description | Sequence | SEQ |
|---|---|---|
| AAV9 VP1 | MAADGYLPDWLEDNLSEGIREWWALKPGAPQPKANQQHQDNARGL | 1 |
| wild-type | VLPGYKYLGPGNGLDKGEPVNAADAAALEHDKAYDQQLKAGDNPY | |
| reference | LKYNHADAEFQERLKEDTSFGGNLGRAVFQAKKRLLEPLGLVEEA | |
| sequence | AKTAPGKKRPVEQSPQEPDSSAGIGKSGAQPAKKRLNFGQTGDTE | |
| (amino acid) | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQTGWVQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| AAV9 VP1 | ATGGCTGCCGATGGTTATCTTCCAGATTGGCTCGAGGACAACCTT | 2 |
| wild-type | AGTGAAGGTATTCGCGAGTGGTGGGCTTTGAAACCTGGAGCCCCT | |
| reference | CAACCCAAGGCAAATCAACAACATCAAGACAACGCTCGAGGTCTT | |
| sequence | GTGCTTCCGGGTTACAAATACCTTGGACCCGGCAACGGACTCGAC | |
| (nucleotide) | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCTATGGACAAGTGGCCACAAACCACCAG | |
| | AGTGCCCAAGCACAGGCGCAGACCGGCTGGGTTCAAAACCAAGGA | |
| | ATACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| AAV2 VP1 | MAADGYLPDWLEDTLSEGIRQWWKLKPGPPPPKPAERHKDDSRGL | 3 |
| wild-type | VLPGYKYLGPFNGLDKGEPVNEADAAALEHDKAYDRQLDSGDNPY | |
| reference | LKYNHADAEFQERLKEDTSFGGNLGRAVFQAKKRVLEPLGLVEEP | |
| sequence | VKTAPGKKRPVEHSPVEPDSSSGTGKAGQQPARKRLNFGQTGDAD | |
| (amino acid) | SVPDPQPLGQPPAAPSGLGTNTMATGSGAPMADNNEGADGVGNSS | |
| | GNWHCDSTWMGDRVITTSTRTWALPTYNNHLYKQISSQSGASNDN | |
| | HYFGYSTPWGYFDFNRFHCHFSPRDWQRLINNNWGFRPKRLNFKL | |
| | FNIQVKEVTQNDGTTTIANNLTSTVQVFTDSEYQLPYVLGSAHQG | |
| | CLPPFPADVFMVPQYGYLTLNNGSQAVGRSSFYCLEYFPSQMLRT | |
| | GNNFTFSYTFEDVPFHSSYAHSQSLDRLMNPLIDQYLYYLSRTNT | |
| | PSGTTTQSRLQFSQAGASDIRDQSRNWLPGPCYRQQRVSKTSADN | |
| | NNSEYSWTGATKYHLNGRDSLVNPGPAMASHKDDEEKFFPQSGVL | |
| | IFGKQGSEKTNVDIEKVMITDEEEIRTTNPVATEQYGSVSTNLQR | |
| | GNRQAATADVNTQGVLPGMVWQDRDVYLQGPIWAKIPHTDGHFHP | |
| | SPLMGGFGLKHPPPQILIKNTPVPANPSTTFSAAKFASFITQYST | |
| | GQVSVEIEWELQKENSKRWNPEIQYTSNYNKSVNVDFTVDTNGVY | |
| | SEPRPIGTRYLTRNL | |
| AAV2 VP1 | ATGGCTGCCGATGGTTATCTTCCAGATTGGCTCGAGGACACTCTC | 4 |
| wild-type | TCTGAAGGAATAAGACAGTGGTGGAAGCTCAAACCTGGCCCACCA | |
| reference | CCACCAAAGCCCGCAGAGCGGCATAAGGACGACAGCAGGGGTCTT | |
| sequence | GTGCTTCCTGGGTACAAGTACCTCGGACCCTTCAACGGACTCGAC | |
| (nucleotide) | AAGGGAGAGCCGGTCAACGAGGCAGACGCCGCGGCCCTCGAGCAC | |
| | GACAAAGCCTACGACCGGCAGCTCGACAGCGGAGACAACCCGTAC | |
| | CTCAAGTACAACCACGCCGACGCGGAGTTTCAGGAGCGCCTTAAA | |
| | GAAGATACGTCTTTTGGGGGCAACCTCGGACGAGCAGTCTTCCAG | |
| | GCGAAAAAGAGGGTTCTTGAACCTCTGGGCCTGGTTGAGGAACCT | |
| | GTTAAGACGGCTCCGGGAAAAAAGAGGCCGGTAGAGCACTCTCCT | |
| | GTGGAGCCAGACTCCTCCTCGGGAACCGGAAAGGCGGGCCAGCAG | |
| | CCTGCAAGAAAAAGATTGAATTTTGGTCAGACTGGAGACGCAGAC | |
| | TCAGTACCTGACCCCCAGCCTCTCGGACAGCCACCAGCAGCCCCC | |
| | TCTGGTCTGGGAACTAATACGATGGCTACAGGCAGTGGCGCACCA | |
| | ATGGCAGACAATAACGAGGGCGCCGACGGAGTGGGTAATTCCTCG | |
| | GGAAATTGGCATTGCGATTCCACATGGATGGGCGACAGAGTCATC | |
| | ACCACCAGCACCCGAACCTGGGCCCTGCCCACCTACAACAACCAC | |
| | CTCTACAAACAAATTTCCAGCCAATCAGGAGCCTCGAACGACAAT | |
| | CACTACTTTGGCTACAGCACCCCTTGGGGGTATTTTGACTTCAAC | |
| | AGATTCCACTGCCACTTTTCACCACGTGACTGGCAAAGACTCATC | |
| | AACAACAACTGGGGATTCCGACCCAAGAGACTCAACTTCAAGCTC | |
| | TTTAACATTCAAGTCAAAGAGGTCACGCAGAATGACGGTACGACG | |
| | ACGATTGCCAATAACCTTACCAGCACGGTTCAGGTGTTTACTGAC | |
| | TCGGAGTACCAGCTCCCGTACGTCCTCGGCTCGGCGCATCAAGGA | |
| | TGCCTCCCGCCGTTCCCAGCAGACGTCTTCATGGTGCCACAGTAT | |
| | GGATACCTCACCCTGAACAACGGGAGTCAGGCAGTAGGACGCTCT | |
| | TCATTTTACTGCCTGGAGTACTTTCCTTCTCAGATGCTGCGTACC | |
| | GGAAACAACTTTACCTTCAGCTACACTTTTGAGGACGTTCCTTTC | |
| | CACAGCAGCTACGCTCACAGCCAGAGTCTGGACCGTCTCATGAAT | |
| | CCTCTCATCGACCAGTACCTGTATTACTTGAGCAGAACAAACACT | |
| | CCAAGTGGAACCACCACGCAGTCAAGGCTTCAGTTTTCTCAGGCC | |
| | GGAGCGAGTGACATTCGGGACCAGTCTAGGAACTGGCTTCCTGGA | |
| | CCCTGTTACCGCCAGCAGCGAGTATCAAAGACATCTGCGGATAAC | |
| | AACAACAGTGAATACTCGTGGACTGGAGCTACCAAGTACCACCTC | |
| | AATGGCAGAGACTCTCTGGTGAATCCGGGCCCGGCCATGGCAAGC | |
| | CACAAGGACGATGAAGAAAAGTTTTTTCCTCAGAGCGGGGTTCTC | |
| | ATCTTTGGGAAGCAAGGCTCAGAGAAAACAAATGTGGACATTGAA | |
| | AAGGTCATGATTACAGACGAAGAGGAAATCAGGACAACCAATCCC | |
| | GTGGCTACGGAGCAGTATGGTTCTGTATCTACCAACCTCCAGAGA | |
| | GGCAACAGACAAGCAGCTACCGCAGATGTCAACACACAAGGCGTT | |
| | CTTCCAGGCATGGTCTGGCAGGACAGAGATGTGTACCTTCAGGGG | |
| | CCCATCTGGGCAAAGATTCCACACACGGACGGACATTTTCACCCC | |
| | TCTCCCCTCATGGGTGGATTCGGACTTAAACACCCTCCTCCACAG | |
| | ATTCTCATCAAGAACACCCCGGTACCTGCGAATCCTTCGACCACC | |
| | TTCAGTGCGGCAAAGTTTGCTTCCTTCATCACACAGTACTCCACG | |
| | GGACAGGTCAGCGTGGAGATCGAGTGGGAGCTGCAGAAGGAAAAC | |
| | AGCAAACGCTGGAATCCCGAAATTCAGTACACTTCCAACTACAAC | |
| | AAGTCTGTTAATGTGGACTTTACTGTGGACACTAATGGCGTGTAT | |
| | TCAGAGCCTCGCCCCATTGGCACCAGATACCTGACTCGTAATCTG | |
| | TAA | |
| AAV5 VP1 | MSFVDHPPDWLEEVGEGLREFLGLEAGPPKPKPNQQHQDQARGLV | 5 |
| wild-type | LPGYNYLGPGNGLDRGEPVNRADEVAREHDISYNEQLEAGDNPYL | |
| reference | KYNHADAEFQEKLADDTSFGGNLGKAVFQAKKRVLEPFGLVEEGA | |
| sequence | KTAPTGKRIDDHFPKRKKARTEEDSKPSTSSDAEAGPSGSQQLQI | |
| (amino acid) | PAQPASSLGADTMSAGGGGPLGDNNQGADGVGNASGDWHCDSTWM | |
| | GDRVVTKSTRTWVLPSYNNHQYREIKSGSVDGSNANAYFGYSTPW | |
| | GYFDFNRFHSHWSPRDWQRLINNYWGFRPRSLRVKIFNIQVKEVT | |
| | VQDSTTTIANNLTSTVQVFTDDDYQLPYVVGNGTEGCLPAFPPQV | |
| | FTLPQYGYATLNRDNTENPTERSSFFCLEYFPSKMLRTGNNFEFT | |
| | YNFEEVPFHSSFAPSQNLFKLANPLVDQYLYRFVSTNNTGGVQFN | |
| | KNLAGRYANTYKNWFPGPMGRTQGWNLGSGVNRASVSAFATTNRM | |
| | ELEGASYQVPPQPNGMTNNLQGSNTYALENTMIFNSQPANPGTTA | |
| | TYLEGNMLITSESETQPVNRVAYNVGGQMATNNQSSTTAPATGTY | |
| | NLQEIVPGSVWMERDVYLQGPIWAKIPETGAHFHPSPAMGGFGLK | |
| | HPPPMMLIKNTPVPGNITSFSDVPVSSFITQYSTGQVTVEMEWEL | |
| | KKENSKRWNPEIQYTNNYNDPQFVDFAPDSTGEYRTTRPIGTRYL | |
| | TRPL | |
| AAV5 VP1 | ATGTCTTTTGTTGATCACCCTCCAGATTGGTTGGAAGAAGTTGGT | 6 |
| wild-type | GAAGGTCTTCGCGAGTTTTTGGGCCTTGAAGCGGGCCCACCGAAA | |
| reference | CCAAAACCCAATCAGCAGCATCAAGATCAAGCCCGTGGTCTTGTG | |
| sequence | CTGCCTGGTTATAACTATCTCGGACCCGGAAACGGGCTCGATCGA | |
| (nucleotide) | GGAGAGCCTGTCAACAGGGCAGACGAGGTCGCGCGAGAGCACGAC | |
| | ATCTCGTACAACGAGCAGCTTGAGGCGGGAGACAACCCCTACCTC | |
| | AAGTACAACCACGCGGACGCCGAGTTTCAGGAGAAGCTCGCCGAC | |
| | GACACATCCTTCGGGGGAAACCTCGGAAAGGCAGTCTTTCAGGCC | |
| | AAGAAAAGGGTTCTCGAACCTTTTGGCCTGGTTGAAGAGGGTGCT | |
| | AAGACGGCCCCTACCGGAAAGCGGATAGACGACCACTTTCCAAAA | |
| | AGAAAGAAGGCTCGGACCGAAGAGGACTCCAAGCCTTCCACCTCG | |
| | TCAGACGCCGAAGCTGGACCCAGCGGATCCCAGCAGCTGCAAATC | |
| | CCAGCCCAACCAGCCTCAAGTTTGGGAGCTGATACAATGTCTGCG | |
| | GGAGGTGGCGGCCCATTGGGCGACAATAACCAAGGTGCCGATGGA | |
| | GTGGGCAATGCCTCGGGAGATTGGCATTGCGATTCCACGTGGATG | |
| | GGGGACAGAGTCGTCACCAAGTCCACCCGAACCTGGGTGCTGCCC | |
| | AGCTACAACAACCACCAGTACCGAGAGATCAAAAGCGGCTCCGTC | |
| | GACGGAAGCAACGCCAACGCCTACTTTGGATACAGCACCCCCTGG | |
| | GGGTACTTTGACTTTAACCGCTTCCACAGCCACTGGAGCCCCCGA | |
| | GACTGGCAAAGACTCATCAACAACTACTGGGGCTTCAGACCCCGG | |
| | TCCCTCAGAGTCAAAATCTTCAACATTCAAGTCAAAGAGGTCACG | |
| | GTGCAGGACTCCACCACCACCATCGCCAACAACCTCACCTCCACC | |
| | GTCCAAGTGTTTACGGACGACGACTACCAGCTGCCCTACGTCGTC | |
| | GGCAACGGGACCGAGGGATGCCTGCCGGCCTTCCCTCCGCAGGTC | |
| | TTTACGCTGCCGCAGTACGGTTACGCGACGCTGAACCGCGACAAC | |
| | ACAGAAAATCCCACCGAGAGGAGCAGCTTCTTCTGCCTAGAGTAC | |
| | TTTCCCAGCAAGATGCTGAGAACGGGCAACAACTTTGAGTTTACC | |
| | TACAACTTTGAGGAGGTGCCCTTCCACTCCAGCTTCGCTCCCAGT | |
| | CAGAACCTGTTCAAGCTGGCCAACCCGCTGGTGGACCAGTACTTG | |
| | TACCGCTTCGTGAGCACAAATAACACTGGCGGAGTCCAGTTCAAC | |
| | AAGAACCTGGCCGGGAGATACGCCAACACCTACAAAAACTGGTTC | |
| | CCGGGGCCCATGGGCCGAACCCAGGGCTGGAACCTGGGCTCCGGG | |
| | GTCAACCGCGCCAGTGTCAGCGCCTTCGCCACGACCAATAGGATG | |
| | GAGCTCGAGGGCGCGAGTTACCAGGTGCCCCCGCAGCCGAACGGC | |
| | ATGACCAACAACCTCCAGGGCAGCAACACCTATGCCCTGGAGAAC | |
| | ACTATGATCTTCAACAGCCAGCCGGCGAACCCGGGCACCACCGCC | |
| | ACGTACCTCGAGGGCAACATGCTCATCACCAGCGAGAGCGAGACG | |
| | CAGCCGGTGAACCGCGTGGCGTACAACGTCGGCGGGCAGATGGCC | |
| | ACCAACAACCAGAGCTCCACCACTGCCCCCGCGACCGGCACGTAC | |
| | AACCTCCAGGAAATCGTGCCCGGCAGCGTGTGGATGGAGAGGGAC | |
| | GTGTACCTCCAAGGACCCATCTGGGCCAAGATCCCAGAGACGGGG | |
| | GCGCACTTTCACCCCTCTCCGGCCATGGGGGGATTCGGACTCAAA | |
| | CACCCACCGCCCATGATGCTCATCAAGAACACGCCTGTGCCCGGA | |
| | AATATCACCAGCTTCTCGGACGTGCCCGTCAGCAGCTTCATCACC | |
| | CAGTACAGCACCGGGCAGGTCACCGTGGAGATGGAGTGGGAGCTC | |
| | AAGAAGGAAAACTCCAAGAGGTGGAACCCAGAGATCCAGTACACA | |
| | AACAACTACAACGACCCCCAGTTTGTGGACTTTGCCCCGGACAGC | |
| | ACCGGGGAATACAGAACCACCAGACCTATCGGAACCCGATACCTT | |
| | ACCCGACCCCTTTAA | |
| AAV8 VP1 | MAADGYLPDWLEDNLSEGIREWWALKPGAPKPKANQQKQDDGRGL | 7 |
| wild-type | VLPGYKYLGPFNGLDKGEPVNAADAAALEHDKAYDQQLQAGDNPY | |
| reference | LRYNHADAEFQERLQEDTSFGGNLGRAVFQAKKRVLEPLGLVEEG | |
| sequence | AKTAPGKKRPVEPSPQRSPDSSTGIGKKGQQPARKRLNFGQTGDS | |
| (amino acid) | ESVPDPQPLGEPPAAPSGVGPNTMAAGGGAPMADNNEGADGVGSS | |
| | SGNWHCDSTWLGDRVITTSTRTWALPTYNNHLYKQISNGTSGGAT | |
| | NDNTYFGYSTPWGYFDFNRFHCHFSPRDWQRLINNNWGFRPKRLS | |
| | FKLFNIQVKEVTQNEGTKTIANNLTSTIQVFTDSEYQLPYVLGSA | |
| | HQGCLPPFPADVFMIPQYGYLTLNNGSQAVGRSSFYCLEYFPSQM | |
| | LRTGNNFQFTYTFEDVPFHSSYAHSQSLDRLMNPLIDQYLYYLSR | |
| | TQTTGGTANTQTLGFSQGGPNTMANQAKNWLPGPCYRQQRVSTTT | |
| | GQNNNSNFAWTAGTKYHLNGRNSLANPGIAMATHKDDEERFFPSN | |
| | GILIFGKQNAARDNADYSDVMLTSEEEIKTTNPVATEEYGIVADN | |
| | LQQQNTAPQIGTVNSQGALPGMVWQNRDVYLQGPIWAKIPHTDGN | |
| | FHPSPLMGGFGLKHPPPQILIKNTPVPADPPTTFNQSKLNSFITQ | |
| | YSTGQVSVEIEWELQKENSKRWNPEIQYTSNYYKSTSVDFAVNTE | |
| | GVYSEPRPIGTRYLTRNL | |
| AAV8 VP1 | ATGGCTGCCGATGGTTATCTTCCAGATTGGCTCGAGGACAACCTC | 8 |
| wild-type | TCTGAGGGCATTCGCGAGTGGTGGGCGCTGAAACCTGGAGCCCCG | |
| reference | AAGCCCAAAGCCAACCAGCAAAAGCAGGACGACGGCCGGGGTCTG | |
| sequence | GTGCTTCCTGGCTACAAGTACCTCGGACCCTTCAACGGACTCGAC | |
| (nucleotide) | AAGGGGGAGCCCGTCAACGCGGCGGACGCAGCGGCCCTCGAGCAC | |
| | GACAAGGCCTACGACCAGCAGCTGCAGGGGGTGACAATCCGTACC | |
| | TGCGGTATAACCACGCCGACGCCGAGTTTCAGGAGCGTCTGCAAG | |
| | AAGATACGTCTTTTGGGGGCAACCTCGGGCGAGCAGTCTTCCAGG | |
| | CCAAGAAGCGGGTTCTCGAACCTCTCGGTCTGGTTGAGGAAGGCG | |
| | CTAAGACGGCTCCTGGAAAGAAGAGACCGGTAGAGCCATCACCCC | |
| | AGCGTTCTCCAGACTCCTCTACGGGCATCGGCAAGAAAGGCCAAC | |
| | AGCCCGCCAGAAAAAGACTCAATTTTGGTCAGACTGGCGACTCAG | |
| | AGTCAGTTCCAGACCCTCAACCTCTCGGAGAACCTCCAGCAGCGC | |
| | CCTCTGGTGTGGGACCTAATACAATGGCTGCAGGCGGTGGCGCAC | |
| | CAATGGCAGACAATAACGAAGGCGCCGACGGAGTGGGTAGTTCCT | |
| | CGGGAAATTGGCATTGCGATTCCACATGGCTGGGCGACAGAGTCA | |
| | TCACCACCAGCACCCGAACCTGGGCCCTGCCCACCTACAACAACC | |
| | ACCTCTACAAGCAAATCTCCAACGGGACATCGGGAGGAGCCACCA | |
| | ACGACAACACCTACTTCGGCTACAGCACCCCCTGGGGGTATTTTG | |
| | ACTTTAACAGATTCCACTGCCACTTTTCACCACGTGACTGGCAGC | |
| | GACTCATCAACAACAACTGGGGATTCCGGCCCAAGAGACTCAGCT | |
| | TCAAGCTCTTCAACATCCAGGTCAAGGAGGTCACGCAGAATGAAG | |
| | GCACCAAGACCATCGCCAATAACCTCACCAGCACCATCCAGGTGT | |
| | TTACGGACTCGGAGTACCAGCTGCCGTACGTTCTCGGCTCTGCCC | |
| | ACCAGGGCTGCCTGCCTCCGTTCCCGGCGGACGTGTTCATGATTC | |
| | CCCAGTACGGCTACCTAACACTCAACAACGGTAGTCAGGCCGTGG | |
| | GACGCTCCTCCTTCTACTGCCTGGAATACTTTCCTTCGCAGATGC | |
| | TGAGAACCGGCAACAACTTCCAGTTTACTTACACCTTCGAGGACG | |
| | TGCCTTTCCACAGCAGCTACGCCCACAGCCAGAGCTTGGACCGGC | |
| | TGATGAATCCTCTGATTGACCAGTACCTGTACTACTTGTCTCGGA | |
| | CTCAAACAACAGGAGGCACGGCAAATACGCAGACTCTGGGCTTCA | |
| | GCCAAGGTGGGCCTAATACAATGGCCAATCAGGCAAAGAACTGGC | |
| | TGCCAGGACCCTGTTACCGCCAACAACGCGTCTCAACGACAACCG | |
| | GGCAAAACAACAATAGCAACTTTGCCTGGACTGCTGGGACCAAAT | |
| | ACCATCTGAATGGAAGAAATTCATTGGCTAATCCTGGCATCGCTA | |
| | TGGCAACACACAAAGACGACGAGGAGCGTTTTTTTCCCAGTAACG | |
| | GGATCCTGATTTTTGGCAAACAAAATGCTGCCAGAGACAATGCGG | |
| | ATTACAGCGATGTCATGCTCACCAGCGAGGAAGAAATCAAAACCA | |
| | CTAACCCTGTGGCTACAGAGGAATACGGTATCGTGGCAGATAACT | |
| | TGCAGCAGCAAAACACGGCTCCTCAAATTGGAACTGTCAACAGCC | |
| | AGGGGGCCTTACCCGGTATGGTCTGGCAGAACCGGGACGTGTACC | |
| | TGCAGGGTCCCATCTGGGCCAAGATTCCTCACACGGACGGCAACT | |
| | TCCACCCGTCTCCGCTGATGGGCGGCTTTGGCCTGAAACATCCTC | |
| | CGCCTCAGATCCTGATCAAGAACACGCCTGTACCTGCGGATCCTC | |
| | CGACCACCTTCAACCAGTCAAAGCTGAACTCTTTCATCACGCAAT | |
| | ACAGCACCGGACAGGTCAGCGTGGAAATTGAATGGGAGCTGCAGA | |
| | AGGAAAACAGCAAGCGCTGGAACCCCGAGATCCAGTACACCTCCA | |
| | ACTACTACAAATCTACAAGTGTGGACTTTGCTGTTAATACAGAAG | |
| | GCGTGTACTCTGAACCCCGCCCCATTGGCACCCGTTACCTCACCC | |
| | GTAATCTGTAA | |
| AAVrh74 VP1 | MAADGYLPDWLEDNLSEGIREWWDLKPGAPKPKANQQKQDNGRGL | 9 |
| wild-type | VLPGYKYLGPFNGLDKGEPVNAADAAALEHDKAYDQQLQAGDNPY | |
| reference | LRYNHADAEFQERLQEDTSFGGNLGRAVFQAKKRVLEPLGLVESP | |
| sequence | VKTAPGKKRPVEPSPQRSPDSSTGIGKKGQQPAKKRLNFGQTGDS | |
| (amino acid) | ESVPDPQPIGEPPAGPSGLGSGTMAAGGGAPMADNNEGADGVGSS | |
| | SGNWHCDSTWLGDRVITTSTRTWALPTYNNHLYKQISNGTSGGST | |
| | NDNTYFGYSTPWGYFDFNRFHCHFSPRDWQRLINNNWGFRPKRLN | |
| | FKLFNIQVKEVTQNEGTKTIANNLTSTIQVFTDSEYQLPYVLGSA | |
| | HQGCLPPFPADVFMIPQYGYLTLNNGSQAVGRSSFYCLEYFPSQM | |
| | LRTGNNFEFSYNFEDVPFHSSYAHSQSLDRLMNPLIDQYLYYLSR | |
| | TQSTGGTAGTQQLLFSQAGPNNMSAQAKNWLPGPCYRQQRVSTTL | |
| | SQNNNSNFAWTGATKYHLNGRDSLVNPGVAMATHKDDEERFFPSS | |
| | GVLMFGKQGAGKDNVDYSSVMLTSEEEIKTTNPVATEQYGVVADN | |
| | LQQQNAAPIVGAVNSQGALPGMVWQNRDVYLQGPIWAKIPHTDGN | |
| | FHPSPLMGGFGLKHPPPQILIKNTPVPADPPTTFNQAKLASFITQ | |
| | YSTGQVSVEIEWELQKENSKRWNPEIQYTSNYYKSTNVDFAVNTE | |
| | GTYSEPRPIGTRYLTRNL | |
| AAVrh74 VP1 | ATGGCTGCCGATGGTTATCTTCCAGATTGGCTCGAGGACAACCTC | 10 |
| wild-type | TCTGAGGGCATTCGCGAGTGGTGGGACCTGAAACCTGGAGCCCCG | |
| reference | AAACCCAAAGCCAACCAGCAAAAGCAGGACAACGGCCGGGGTCTG | |
| sequence | GTGCTTCCTGGCTACAAGTACCTCGGACCCTTCAACGGACTCGAC | |
| (nucleotide) | AAGGGGGAGCCCGTCAACGCGGCGGACGCAGCGGCCCTCGAGCAC | |
| | GACAAGGCCTACGACCAGCAGCTCCAAGCGGGTGACAATCCGTAC | |
| | CTGCGGTATAATCACGCCGACGCCGAGTTTCAGGAGCGTCTGCAA | |
| | GAAGATACGTCTTTTGGGGGCAACCTCGGGCGCGCAGTCTTCCAG | |
| | GCCAAAAAGCGGGTTCTCGAACCTCTGGGCCTGGTTGAATCGCCG | |
| | GTTAAGACGGCTCCTGGAAAGAAGAGGCCGGTAGAGCCATCACCC | |
| | CAGCGCTCTCCAGACTCCTCTACGGGCATCGGCAAGAAAGGCCAG | |
| | CAGCCCGCAAAAAAGAGACTCAATTTTGGGCAGACTGGCGACTCA | |
| | GAGTCAGTCCCCGACCCTCAACCAATCGGAGAACCACCAGCAGGC | |
| | CCCTCTGGTCTGGGATCTGGTACAATGGCTGCAGGCGGTGGCGCT | |
| | CCAATGGCAGACAATAACGAAGGCGCCGACGGAGTGGGTAGTTCC | |
| | TCAGGAAATTGGCATTGCGATTCCACATGGCTGGGCGACAGAGTC | |
| | ATCACCACCAGCACCCGCACCTGGGCCCTGCCCACCTACAACAAC | |
| | CACCTCTACAAGCAAATCTCCAACGGGACCTCGGGAGGAAGCACC | |
| | AACGACAACACCTACTTCGGCTACAGCACCCCCTGGGGGTATTTT | |
| | GACTTCAACAGATTCCACTGCCACTTTTCACCACGTGACTGGCAG | |
| | CGACTCATCAACAACAACTGGGGATTCCGGCCCAAGAGGCTCAAC | |
| | TTCAAGCTCTTCAACATCCAAGTCAAGGAGGTCACGCAGAATGAA | |
| | GGCACCAAGACCATCGCCAATAACCTTACCAGCACGATTCAGGTC | |
| | TTTACGGACTCGGAATACCAGCTCCCGTACGTGCTCGGCTCGGCG | |
| | CACCAGGGCTGCCTGCCTCCGTTCCCGGCGGACGTCTTCATGATT | |
| | CCTCAGTACGGGTACCTGACTCTGAACAATGGCAGTCAGGCTGTG | |
| | GGCCGGTCGTCCTTCTACTGCCTGGAGTACTTTCCTTCTCAAATG | |
| | CTGAGAACGGGCAACAACTTTGAATTCAGCTACAACTTCGAGGAC | |
| | GTGCCCTTCCACAGCAGCTACGCGCACAGCCAGAGCCTGGACCGG | |
| | CTGATGAACCCTCTCATCGACCAGTACTTGTACTACCTGTCCCGG | |
| | ACTCAAAGCACGGGGGGTACTGCAGGAACTCAGCAGTTGCTATTT | |
| | TCTCAGGCCGGGCCTAACAACATGTCGGCTCAGGCCAAGAACTGG | |
| | CTACCCGGTCCCTGCTACCGGCAGCAACGTGTCTCCACGACACTG | |
| | TCGCAGAACAACAACAGCAACTTTGCCTGGACGGGTGCCACCAAG | |
| | TATCATCTGAATGGCAGAGACTCTCTGGTGAATCCTGGCGTTGCC | |
| | ATGGCTACCCACAAGGACGACGAAGAGCGATTTTTTCCATCCAGC | |
| | GGAGTCTTAATGTTTGGGAAACAGGGAGCTGGAAAAGACAACGTG | |
| | GACTATAGCAGCGTGATGCTAACCAGCGAGGAAGAAATAAAGACC | |
| | ACCAACCCAGTGGCCACAGAACAGTACGGCGTGGTGGCCGATAAC | |
| | CTGCAACAGCAAAACGCCGCTCCTATTGTAGGGGCCGTCAATAGT | |
| | CAAGGAGCCTTACCTGGCATGGTGTGGCAGAACCGGGACGTGTAC | |
| | CTGCAGGGTCCCATCTGGGCCAAGATTCCTCATACGGACGGCAAC | |
| | TTTCATCCCTCGCCGCTGATGGGAGGCTTTGGACTGAAGCATCCG | |
| | CCTCCTCAGATCCTGATTAAAAACACACCTGTTCCCGCGGATCCT | |
| | CCGACCACCTTCAATCAGGCCAAGCTGGCTTCTTTCATCACGCAG | |
| | TACAGTACCGGCCAGGTCAGCGTGGAGATCGAGTGGGAGCTGCAG | |
| | AAGGAGAACAGCAAACGCTGGAACCCAGAGATTCAGTACACTTCC | |
| | AACTACTACAAATCTACAAATGTGGACTTTGCTGTCAATACTGAG | |
| | GGTACTTATTCCGAGCCTCGCCCCATTGGCACCCGTTACCTCACC | |
| | CGTAATCTGTAA | |
| AAVrh74 VP1 | MAADGYLPDWLEDNLSEGIREWWDLKPGAPKPKANQQKQDNGRGL | |
| wild-type | VLPGYKYLGPFNGLDKGEPVNAADAAALEHDKAYDQQLQAGDNPY | |
| alternative | LRYNHADAEFQERLQEDTSFGGNLGRAVFQAKKRVLEPLGLVESP | |
| reference | VKTAPGKKRPVEPSPQRSPDSSTGIGKKGQQPAKKRLNFGQTGDS | |
| sequence | ESVPDPQPIGEPPAGPSGLGSGTMAAGGGAPMADNNEGADGVGSS | |
| (amino acid) | SGNWHCDSTWLGDRVITTSTRTWALPTYNNHLYKQISNGTSGGST | |
| | NDNTYFGYSTPWGYFDFNRFHCHFSPRDWQRLINNNWGFRPKRLN | |
| | FKLFNIQVKEVTQNEGTKTIANNLTSTIQVFTDSEYQLPYVLGSA | |
| | HQGCLPPFPADVFMIPQYGYLTLNNGSQAVGRSSFYCLEYFPSQM | |
| | LRTGNNFEFSYNFEDVPFHSSYAHSQSLDRLMNPLIDQYLYYLSR | |
| | TQSTGGTAGTQQLLFSQAGPNNMSAQAKNWLPGPCYRQQRVSTTL | |
| | SQNNNSNFAWTGATKYHLNGRDSLVNPGVAMATHKDDEERFFPSS | |
| | GVLMFGKQGAGKDNVDYSSVMLTSEEEIKTTNPVATEQYGVVADN | |
| | LQQQNAAPIVGAVNSQGALPGMVWQNRDVYLQGPIWAKIPHTDGN | |
| | FHPSPLMGGFGLKHPPPQILIKNTPVPADPPTTFTKAKLASFITQ | |
| | YSTGQVSVEIEWELQKENSKRWNPEIQYTSNYYKSTNVDFAVNTE | |
| | GTYSEPRPIGTRYLTRNL | 11 |
| VAR-1 | MAADGYLPDWLEDNLSEGIREWWALKPGAPQPKANQQHQDNARGL | 12 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQTQAQAGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-2 | MAADGYLPDWLEDNLSEGIREWWALKPGAPQPKANQQHQDNARGL | 13 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRENVDADKVMITNEEEIKTTNPVATESYGTVATNHQ | |
| | SAQAQASVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-3 | MAADGYLPDWLEDNLSEGIREWWALKPGAPQPKANQQHQDNARGL | 14 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SASAQAQAGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-4 | MAADGYLPDWLEDNLSEGIREWWALKPGAPQPKANQQHQDNARGL | 15 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTYRDNVDADKVMITNEEEIKTTNPVATESYGTVATNHQ | |
| | SAQAQAQVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-5 | MAADGYLPDWLEDNLSEGIREWWALKPGAPQPKANQQHQDNARGL | 16 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVSITNEEEIKTTNPVATESYGTVATNIQ | |
| | SAQAQAIVGALQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-6 | MAADGYLPDWLEDNLSEGIREWWALKPGAPQPKANQQHQDNARGL | 17 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVNITNEEEIKTTNPVATESYGVVATNHQ | |
| | SAQAQAQSGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-7 | MAADGYLPDWLEDNLSEGIREWWALKPGAPQPKANQQHQDNARGL | 18 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAGAQAQSGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-8 | MAADGYLPDWLEDNLSEGIREWWALKPGAPQPKANQQHQDNARGL | 19 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESHGVVCTNHQ | |
| | SAQAQAQVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-9 | MAADGYLPDWLEDNLSEGIREWWALKPGAPQPKANQQHQDNARGL | 20 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMLTNEEEIKTTNPVATESYGVVATNHQ | |
| | SAQTQAQAGALQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-10 | MAADGYLPDWLEDNLSEGIREWWALKPGAPQPKANQQHQDNARGL | 21 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVAITNEEEIKTTNPVATESYGVVATNMQ | |
| | SAQAQASVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-11 | MAADGYLPDWLEDNLSEGIREWWALKPGAPQPKANQQHQDNARGL | 22 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SANAQAQAGALQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-12 | MAADGYLPDWLEDNLSEGIREWWALKPGAPQPKANQQHQDNARGL | 23 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVVITNEEEIKTTNPVATESYGVVATNHQ | |
| | SAQAQAQSGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-13 | MAADGYLPDWLEDNLSEGIREWWALKPGAPQPKANQQHQDNARGL | 24 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMQTNEEEIKTTNPVATESYGTVATNHQ | |
| | SAQAQAQVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-14 | MAADGYLPDWLEDNLSEGIREWWALKPGAPQPKANQQHQDNARGL | 25 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGTVATNHQ | |
| | SANTQASVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-15 | MAADGYLPDWLEDNLSEGIREWWALKPGAPQPKANQQHQDNARGL | 26 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVQITNEEEIKTTNPVATESYGVVATNQQ | |
| | SAQAQAQVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-16 | MAADGYLPDWLEDNLSEGIREWWALKPGAPQPKANQQHQDNARGL | 27 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAIRGALQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-17 | MAADGYLPDWLEDNLSEGIREWWALKPGAPQPKANQQHQDNARGL | 28 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVCITNEEEIKTTNPVATESYGVVATNHQ | |
| | SAQAQAQAGALQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-18 | MAADGYLPDWLEDNLSEGIREWWALKPGAPQPKANQQHQDNARGL | 29 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTTRDNVDADKVMITNEEEIKTTNPVATESYGTVATNHQ | |
| | SAQAQAQVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-19 | MAADGYLPDWLEDNLSEGIREWWALKPGAPQPKANQQHQDNARGL | 30 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVAINNQ | |
| | SAQAQAQVGALQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-20 | MAADGYLPDWLEDNLSEGIREWWALKPGAPQPKANQQHQDNARGL | 31 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTARDNVDADKVMITNEEEIKTTNPVATESYGVVATNHQ | |
| | SAQAQAQSGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-21 | MAADGYLPDWLEDNLSEGIREWWALKPGAPQPKANQQHQDNARGL | 32 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGNDNVDADKVMITNEEEIKTTNPVATESTGVVATNHQ | |
| | SAQAQASVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-22 | MAADGYLPDWLEDNLSEGIREWWALKPGAPQPKANQQHQDNARGL | 33 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGKDNVDADKVMITNEEEIKTTNPVATESYGVVATNHQ | |
| | SAQSQAQAGALQSQGILPGMWWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-23 | MAADGYLPDWLEDNLSEGIREWWALKPGAPQPKANQQHQDNARGL | 34 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMISNEEEIKTTNPVATESYGVVATNIQ | |
| | SAQAQASVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-24 | MAADGYLPDWLEDNLSEGIREWWALKPGAPQPKANQQHQDNARGL | 35 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNIQ | |
| | SAQAQASVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-25 | MAADGYLPDWLEDNLSEGIREWWALKPGAPQPKANQQHQDNARGL | 36 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMISNEEEIKTTNPVATESYGVVATNHQ | |
| | SAQAQAQAGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-26 | MAADGYLPDWLEDNLSEGIREWWALKPGAPQPKANQQHQDNARGL | 37 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVAITNEEEIKTTNPVATESYGVVATNHQ | |
| | SAQAQASVGALQTQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-27 | MAADGYLPDWLEDNLSEGIREWWALKPGAPQPKANQQHQDNARGL | 38 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESTGVVATNHQ | |
| | SAQAQASVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-28 | MAADGYLPDWLEDNLSEGIREWWALKPGAPQPKANQQHQDNARGL | 39 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNSDADKVMITNEEEIKTTNPVATESYGTVATNHQ | |
| | SAQAQAQVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-29 | MAADGYLPDWLEDNLSEGIREWWALKPGAPQPKANQQHQDNARGL | 40 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGTVATNHQ | |
| | SHNTQAIVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-30 | MAADGYLPDWLEDNLSEGIREWWALKPGAPQPKANQQHQDNARGL | 41 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADNVMITNEEEIKTTNPVATESYGVVATNLQ | |
| | SAQTQASVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-31 | MAADGYLPDWLEDNLSEGIREWWALKPGAPQPKANQQHQDNARGL | 42 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGTVATNLQ | |
| | SAQAQAQVGALQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-32 | MAADGYLPDWLEDNLSEGIREWWALKPGAPQPKANQQHQDNARGL | 43 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SATAQAQAGALQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-33 | MAADGYLPDWLEDNLSEGIREWWALKPGAPQPKANQQHQDNARGL | 44 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESHGVVAMNHQ | |
| | SAQAQASVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-34 | MAADGYLPDWLEDNLSEGIREWWALKPGAPQPKANQQHQDNARGL | 45 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTFRDNVDADKVMITNEEEIKTTNPVATESYGTVATNHQ | |
| | SAQAQAQVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-35 | MAADGYLPDWLEDNLSEGIREWWALKPGAPQPKANQQHQDNARGL | 46 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATEWYGVVATNHQ | |
| | SSQTQAQVGALQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-36 | MAADGYLPDWLEDNLSEGIREWWALKPGAPQPKANQQHQDNARGL | 47 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGKDNVDADKVMITNEEEIKTTNPVATESYGVVATNHQ | |
| | SAQSQAQSGALQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-37 | MAADGYLPDWLEDNLSEGIREWWALKPGAPQPKANQQHQDNARGL | 48 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTIRDNVDADKVMITNEEEIKTTNPVATESYGVVATNHQ | |
| | SAQAQAQSGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-38 | MAADGYLPDWLEDNLSEGIREWWALKPGAPQPKANQQHQDNARGL | 49 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTHRDNVDADKVMITNEEEIKTTNPVATESYGTVATNHQ | |
| | SAQAQAQVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-39 | MAADGYLPDWLEDNLSEGIREWWALKPGAPQPKANQQHQDNARGL | 50 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVQITNEEEIKTTNPVATESYGVVAINHQ | |
| | SAQAQAQVGALQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-40 | MAADGYLPDWLEDNLSEGIREWWALKPGAPQPKANQQHQDNARGL | 51 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQVGALQTQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-41 | MAADGYLPDWLEDNLSEGIREWWALKPGAPQPKANQQHQDNARGL | 52 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESTGVVATNHQ | |
| | SAQSQAAVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-42 | MAADGYLPDWLEDNLSEGIREWWALKPGAPQPKANQQHQDNARGL | 53 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGTVATNNQ | |
| | SAQAQAQVGALQSQGILPGMWWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-43 | MAADGYLPDWLEDNLSEGIREWWALKPGAPQPKANQQHQDNARGL | 54 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SANAQAQVGALQTQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-44 | MAADGYLPDWLEDNLSEGIREWWALKPGAPQPKANQQHQDNARGL | 55 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SANAQAQAGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-45 | MAADGYLPDWLEDNLSEGIREWWALKPGAPQPKANQQHQDNARGL | 56 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGTVATNHQ | |
| | SANTQANVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-46 | MAADGYLPDWLEDNLSEGIREWWALKPGAPQPKANQQHQDNARGL | 57 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGLDNVDADKVMITNEEEIKTTNPVATESYGTVATNHQ | |
| | SAQAQAQVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-47 | MAADGYLPDWLEDNLSEGIREWWALKPGAPQPKANQQHQDNARGL | 58 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADNVMITNEEEIKTTNPVATESYGVVATNHQ | |
| | SANAQAQAGALQSQGVLPGMVWQNRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-48 | MAADGYLPDWLEDNLSEGIREWWALKPGAPQPKANQQHQDNARGL | 59 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGTVATNHQ | |
| | QSQAQAQVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-49 | MAADGYLPDWLEDNLSEGIREWWALKPGAPQPKANQQHQDNARGL | 60 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGTVATNHQ | |
| | SANTQAAVGALQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-50 | MAADGYLPDWLEDNLSEGIREWWALKPGAPQPKANQQHQDNARGL | 61 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SSQAQAQSGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-51 | MAADGYLPDWLEDNLSEGIREWWALKPGAPQPKANQQHQDNARGL | 62 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGTVATNHQ | |
| | SANTQAQVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-52 | MAADGYLPDWLEDNLSEGIREWWALKPGAPQPKANQQHQDNARGL | 63 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTNRDNVDADKVMITNEEEIKTTNPVATESYGTVATNHQ | |
| | SSQTQAQVGALQSQGILPGMWWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-53 | MAADGYLPDWLEDNLSEGIREWWALKPGAPQPKANQQHQDNARGL | 64 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADLVQITNEEEIKTTNPVATESYGVVATNHQ | |
| | SAQAQAQAGALQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-54 | MAADGYLPDWLEDNLSEGIREWWALKPGAPQPKANQQHQDNARGL | 65 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATEWYGTVNTNHQ | |
| | SAQAQAQVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-55 | MAADGYLPDWLEDNLSEGIREWWALKPGAPQPKANQQHQDNARGL | 66 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNNQ | |
| | SAQAQAQVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-60 | MAADGYLPDWLEDNLSEGIREWWALKPGAPQPKANQQHQDNARGL | 67 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVAITNEEEIKTTNPVATESYGTVATNHQ | |
| | QTQAQAQVGALQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-61 | MAADGYLPDWLEDNLSEGIREWWALKPGAPQPKANQQHQDNARGL | 68 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVTITNEEEIKTTNPVATESYGVVATNHQ | |
| | SSNAQAQSGALQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-57 | MAADGYLPDWLEDNLSEGIREWWALKPGAPQPKANQQHQDNARGL | 69 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVIITNEEEIKTTNPVATESYGSVATNHQ | |
| | SAQAQAATGWLQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-56 | MAADGYLPDWLEDNLSEGIREWWALKPGAPQPKANQQHQDNARGL | 70 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGSVATNLQ | |
| | SAQAQAQVGYLQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-58 | MAADGYLPDWLEDNLSEGIREWWALKPGAPQPKANQQHQDNARGL | 71 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGSVATNHQ | |
| | SAQAQPQTGWLQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-59 | MAADGYLPDWLEDNLSEGIREWWALKPGAPQPKANQQHQDNARGL | 72 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATWSYGQVATNNQ | |
| | SAQAQAQVGALQSQGVLPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-62 | MAADGYLPDWLEDNLSEGIREWWALKPGAPQPKANQQHQDNARGL | 73 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATSSYGQVATNMQ | |
| | SAQAQAQVGALQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-1 | ATGGCTGCCGATGGTTATCTTCCAGATTGGCTCGAGGACAACCTT | 74 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCTATGGAGTTGTGGCCACAAACCACCAG | |
| | AGTGCCCAAACTCAGGCGCAGGCCGGCGCTTTGCAAAGCCAAGGA | |
| | GTACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-2 | ATGGCTGCCGATGGTTATCTTCCAGATTGGCTCGAGGACAACCTT | 75 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGAAAACGTGGATGCG | |
| | GACAAAGTCATGATAACCAACGAAGAAGAAATTAAAACTACTAAC | |
| | CCGGTAGCAACGGAGTCCTATGGAACTGTGGCCACAAACCACCAG | |
| | AGTGCCCAAGCACAGGCGTCTGTTGGCGCTCTTCAATCCCAAGGA | |
| | GTCCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-3 | ATGGCTGCCGATGGTTATCTTCCAGATTGGCTCGAGGACAACCTT | 76 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCTATGGAGTCGTGGCCACAAACCACCAG | |
| | AGTGCCTCGGCACAGGCGCAGGCAGGCGCCTTACAATCGCAAGGA | |
| | GTTCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-4 | ATGGCTGCCGATGGTTATCTTCCAGATTGGCTCGAGGACAACCTT | 77 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTTATAGAGACAACGTGGATGCG | |
| | GACAAAGTCATGATAACCAACGAAGAAGAAATTAAAACTACTAAC | |
| | CCGGTAGCAACGGAGTCCTATGGAACGGTGGCCACAAACCACCAG | |
| | AGTGCCCAAGCACAGGCGCAGGTCGGCGCCCTGCAAAGCCAAGGA | |
| | GTTCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-5 | ATGGCTGCCGATGGTTATCTTCCAGATTGGCTCGAGGACAACCTT | 78 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCAGTATAACCAACGAAGAAGAAATTAAAACTACTAAC | |
| | CCGGTAGCAACGGAGTCCTATGGAACCGTGGCCACAAACATTCAG | |
| | AGTGCCCAAGCACAGGCGATAGTAGGCGCTCTACAAAGCCAAGGA | |
| | ATACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-6 | ATGGCTGCCGATGGTTATCTTCCAGATTGGCTCGAGGACAACCTT | 79 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCAATATAACCAACGAAGAAGAAATTAAAACTACTAAC | |
| | CCGGTAGCAACGGAGTCCTATGGAGTGGTGGCCACAAACCACCAG | |
| | AGTGCCCAAGCACAGGCGCAGTCGGGCGCCTTGCAATCCCAAGGA | |
| | GTTCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-7 | ATGGCTGCCGATGGTTATCTTCCAGATTGGCTCGAGGACAACCTT | 80 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCTATGGAGTTGTGGCCACAAACCACCAG | |
| | AGTGCCGGTGCACAGGCGCAGAGCGGCGCTCTCCAAAGTCAAGGA | |
| | GTCCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-8 | ATGGCTGCCGATGGTTATCTTCCAGATTGGCTCGAGGACAACCTT | 81 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCCATGGAGTTGTGTGCACAAACCACCAG | |
| | AGTGCCCAAGCACAGGCGCAGGTGGGCGCCTTGCAATCTCAAGGA | |
| | GTACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-9 | ATGGCTGCCGATGGTTATCTTCCAGATTGGCTCGAGGACAACCTT | 82 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGCTGACCAACGAAGAAGAAATTAAAACTACTAAC | |
| | CCGGTAGCAACGGAGTCCTATGGAGTTGTGGCCACAAACCACCAG | |
| | AGTGCCCAAACCCAGGCGCAGGCGGGCGCCTTACAATCCCAAGGA | |
| | ATACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-10 | ATGGCTGCCGATGGTTATCTTCCAGATTGGCTCGAGGACAACCTT | 83 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCGCTATAACCAACGAAGAAGAAATTAAAACTACTAAC | |
| | CCGGTAGCAACGGAGTCCTATGGAGTCGTGGCCACAAACATGCAG | |
| | AGTGCCCAAGCACAGGCGTCCGTTGGCGCGCTGCAAAGCCAAGGA | |
| | GTGCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-11 | ATGGCTGCCGATGGTTATCTTCCAGATTGGCTCGAGGACAACCTT | 84 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCTATGGAGTTGTGGCCACAAACCACCAG | |
| | AGTGCCAACGCACAGGCGCAGGCGGGCGCATTGCAATCACAAGGA | |
| | ATACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-12 | ATGGCTGCCGATGGTTATCTTCCAGATTGGCTCGAGGACAACCTT | 85 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCGTAATAACCAACGAAGAAGAAATTAAAACTACTAAC | |
| | CCGGTAGCAACGGAGTCCTATGGAGTGGTGGCCACAAACCACCAG | |
| | AGTGCCCAAGCACAGGCGCAGAGTGGCGCTCTGCAATCACAAGGA | |
| | GTCCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-13 | ATGGCTGCCGATGGTTATCTTCCAGATTGGCTCGAGGACAACCTT | 86 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGCAGACCAACGAAGAAGAAATTAAAACTACTAAC | |
| | CCGGTAGCAACGGAGTCCTATGGAACTGTGGCCACAAACCACCAG | |
| | AGTGCCCAAGCACAGGCGCAGGTGGGCGCCCTACAAAGTCAAGGA | |
| | GTTCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-14 | ATGGCTGCCGATGGTTATCTTCCAGATTGGCTCGAGGACAACCTT | 87 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCTATGGAACCGTGGCCACAAACCACCAG | |
| | AGTGCCAATACGCAGGCGTCCGTAGGCGCACTGCAAAGTCAAGGA | |
| | GTACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-15 | ATGGCTGCCGATGGTTATCTTCCAGATTGGCTCGAGGACAACCTT | 88 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCCAGATAACCAACGAAGAAGAAATTAAAACTACTAAC | |
| | CCGGTAGCAACGGAGTCCTATGGAGTCGTGGCCACAAACCAGCAG | |
| | AGTGCCCAAGCACAGGCGCAGGTCGGCGCTCTTCAAAGCCAAGGA | |
| | GTACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-16 | ATGGCTGCCGATGGTTATCTTCCAGATTGGCTCGAGGACAACCTT | 89 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCTATGGAGTCGTGGCCACAAACCACCAG | |
| | AGTGCCCAAGCACAGGCGATTAGGGGCGCCTTACAATCGCAAGGA | |
| | ATACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-17 | ATGGCTGCCGATGGTTATCTTCCAGATTGGCTCGAGGACAACCTT | 90 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCTGCATAACCAACGAAGAAGAAATTAAAACTACTAAC | |
| | CCGGTAGCAACGGAGTCCTATGGAGTGGTGGCCACAAACCACCAG | |
| | AGTGCCCAAGCACAGGCGCAGGCGGGCGCTCTACAAAGTCAAGGA | |
| | ATACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-18 | ATGGCTGCCGATGGTTATCTTCCAGATTGGCTCGAGGACAACCTT | 91 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTACAAGAGACAACGTGGATGCG | |
| | GACAAAGTCATGATAACCAACGAAGAAGAAATTAAAACTACTAAC | |
| | CCGGTAGCAACGGAGTCCTATGGAACGGTGGCCACAAACCACCAG | |
| | AGTGCCCAAGCACAGGCGCAGGTTGGCGCCCTTCAATCACAAGGA | |
| | GTTCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-19 | ATGGCTGCCGATGGTTATCTTCCAGATTGGCTCGAGGACAACCTT | 92 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCTATGGAGTCGTGGCCATTAACAATCAG | |
| | AGTGCCCAAGCACAGGCGCAGGTCGGCGCGCTCCAAAGTCAAGGA | |
| | ATACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-20 | ATGGCTGCCGATGGTTATCTTCCAGATTGGCTCGAGGACAACCTT | 93 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGCGAGAGACAACGTGGATGCG | |
| | GACAAAGTCATGATAACCAACGAAGAAGAAATTAAAACTACTAAC | |
| | CCGGTAGCAACGGAGTCCTATGGAGTTGTGGCCACAAACCACCAG | |
| | AGTGCCCAAGCACAGGCGCAGAGTGGCGCTCTACAATCGCAAGGA | |
| | GTCCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-21 | ATGGCTGCCGATGGTTATCTTCCAGATTGGCTCGAGGACAACCTT | 94 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAATGACAACGTGGATGCG | |
| | GACAAAGTCATGATAACCAACGAAGAAGAAATTAAAACTACTAAC | |
| | CCGGTAGCAACGGAGTCCACCGGAGTTGTGGCCACAAACCACCAG | |
| | AGTGCCCAAGCACAGGCGTCAGTTGGCGCATTACAAAGTCAAGGA | |
| | GTTCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-22 | ATGGCTGCCGATGGTTATCTTCCAGATTGGCTCGAGGACAACCTT | 95 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAAAGACAACGTGGATGCG | |
| | GACAAAGTCATGATAACCAACGAAGAAGAAATTAAAACTACTAAC | |
| | CCGGTAGCAACGGAGTCCTATGGAGTTGTGGCCACAAACCACCAG | |
| | AGTGCCCAAAGCCAGGCGCAGGCTGGCGCGCTCCAATCTCAAGGA | |
| | ATACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-23 | ATGGCTGCCGATGGTTATCTTCCAGATTGGCTCGAGGACAACCTT | 96 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAAGTAACGAAGAAGAAATTAAAACTACTAAC | |
| | CCGGTAGCAACGGAGTCCTATGGAGTCGTGGCCACAAACATCCAG | |
| | AGTGCCCAAGCACAGGCGAGTGTGGGCGCGTTGCAATCACAAGGA | |
| | GTACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-24 | ATGGCTGCCGATGGTTATCTTCCAGATTGGCTCGAGGACAACCTT | 97 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCTATGGAGTTGTGGCCACAAACATTCAG | |
| | AGTGCCCAAGCACAGGCGTCCGTCGGCGCACTTCAATCTCAAGGA | |
| | GTTCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-25 | ATGGCTGCCGATGGTTATCTTCCAGATTGGCTCGAGGACAACCTT | 98 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATTTCCAACGAAGAAGAAATTAAAACTACTAAC | |
| | CCGGTAGCAACGGAGTCCTATGGAGTAGTGGCCACAAACCACCAG | |
| | AGTGCCCAAGCACAGGCGCAGGCGGGCGCGTTGCAAAGTCAAGGA | |
| | GTGCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-26 | ATGGCTGCCGATGGTTATCTTCCAGATTGGCTCGAGGACAACCTT | 99 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCGCGATAACCAACGAAGAAGAAATTAAAACTACTAAC | |
| | CCGGTAGCAACGGAGTCCTATGGAGTCGTGGCCACAAACCACCAG | |
| | AGTGCCCAAGCACAGGCGTCAGTGGGCGCACTACAAACACAAGGA | |
| | GTGCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-27 | ATGGCTGCCGATGGTTATCTTCCAGATTGGCTCGAGGACAACCTT | 100 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCACTGGAGTAGTGGCCACAAACCACCAG | |
| | AGTGCCCAAGCACAGGCGTCCGTAGGCGCTTTACAATCACAAGGA | |
| | GTTCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-28 | ATGGCTGCCGATGGTTATCTTCCAGATTGGCTCGAGGACAACCTT | 101 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACAGCGATGCG | |
| | GACAAAGTCATGATAACCAACGAAGAAGAAATTAAAACTACTAAC | |
| | CCGGTAGCAACGGAGTCCTATGGAACGGTGGCCACAAACCACCAG | |
| | AGTGCCCAAGCACAGGCGCAGGTCGGCGCACTGCAATCTCAAGGA | |
| | GTCCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-29 | ATGGCTGCCGATGGTTATCTTCCAGATTGGCTCGAGGACAACCTT | 102 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCTATGGAACTGTGGCCACAAACCACCAG | |
| | AGTCACAACACTCAGGCGATCGTAGGCGCACTGCAAAGCCAAGGA | |
| | GTGCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-30 | ATGGCTGCCGATGGTTATCTTCCAGATTGGCTCGAGGACAACCTT | 103 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAATGTCATGATAACCAACGAAGAAGAAATTAAAACTACTAAC | |
| | CCGGTAGCAACGGAGTCCTATGGAGTGGTGGCCACAAACTTACAG | |
| | AGTGCCCAAACCCAGGCGTCAGTAGGCGCGCTCCAATCACAAGGA | |
| | GTACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-31 | ATGGCTGCCGATGGTTATCTTCCAGATTGGCTCGAGGACAACCTT | 104 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCTATGGAACCGTGGCCACAAACCTTCAG | |
| | AGTGCCCAAGCACAGGCGCAGGTTGGCGCTCTACAAAGTCAAGGA | |
| | ATACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-32 | ATGGCTGCCGATGGTTATCTTCCAGATTGGCTCGAGGACAACCTT | 105 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCTATGGAGTGGTGGCCACAAACCACCAG | |
| | AGTGCCACAGCACAGGCGCAGGCCGGCGCCTTGCAATCACAAGGA | |
| | ATACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-33 | ATGGCTGCCGATGGTTATCTTCCAGATTGGCTCGAGGACAACCTT | 106 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCCATGGAGTAGTGGCCATGAACCACCAG | |
| | AGTGCCCAAGCACAGGCGAGCGTAGGCGCCCTTCAATCTCAAGGA | |
| | GTCCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-34 | ATGGCTGCCGATGGTTATCTTCCAGATTGGCTCGAGGACAACCTT | 107 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTTTTAGAGACAACGTGGATGCG | |
| | GACAAAGTCATGATAACCAACGAAGAAGAAATTAAAACTACTAAC | |
| | CCGGTAGCAACGGAGTCCTATGGAACCGTGGCCACAAACCACCAG | |
| | AGTGCCCAAGCACAGGCGCAGGTAGGCGCGCTGCAAAGCCAAGGA | |
| | GTACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-35 | ATGGCTGCCGATGGTTATCTTCCAGATTGGCTCGAGGACAACCTT | 108 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTGGTATGGAGTGGTGGCCACAAACCACCAG | |
| | AGTTCCCAAACACAGGCGCAGGTTGGCGCTTTGCAAAGCCAAGGA | |
| | ATACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-36 | ATGGCTGCCGATGGTTATCTTCCAGATTGGCTCGAGGACAACCTT | 109 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAAAGACAACGTGGATGCG | |
| | GACAAAGTCATGATAACCAACGAAGAAGAAATTAAAACTACTAAC | |
| | CCGGTAGCAACGGAGTCCTATGGAGTAGTGGCCACAAACCACCAG | |
| | AGTGCCCAAAGTCAGGCGCAGAGCGGCGCCCTGCAATCCCAAGGA | |
| | ATACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-37 | ATGGCTGCCGATGGTTATCTTCCAGATTGGCTCGAGGACAACCTT | 110 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTATAAGAGACAACGTGGATGCG | |
| | GACAAAGTCATGATAACCAACGAAGAAGAAATTAAAACTACTAAC | |
| | CCGGTAGCAACGGAGTCCTATGGAGTGGTGGCCACAAACCACCAG | |
| | AGTGCCCAAGCACAGGCGCAGAGTGGCGCGCTGCAAAGCCAAGGA | |
| | GTCCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-38 | ATGGCTGCCGATGGTTATCTTCCAGATTGGCTCGAGGACAACCTT | 111 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTCATAGAGACAACGTGGATGCG | |
| | GACAAAGTCATGATAACCAACGAAGAAGAAATTAAAACTACTAAC | |
| | CCGGTAGCAACGGAGTCCTATGGAACCGTGGCCACAAACCACCAG | |
| | AGTGCCCAAGCACAGGCGCAGGTAGGCGCGCTTCAAAGCCAAGGA | |
| | GTACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-39 | ATGGCTGCCGATGGTTATCTTCCAGATTGGCTCGAGGACAACCTT | 112 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCCAAATAACCAACGAAGAAGAAATTAAAACTACTAAC | |
| | CCGGTAGCAACGGAGTCCTATGGAGTCGTGGCCATAAACCACCAG | |
| | AGTGCCCAAGCACAGGCGCAGGTTGGCGCCCTGCAATCACAAGGA | |
| | ATACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-40 | ATGGCTGCCGATGGTTATCTTCCAGATTGGCTCGAGGACAACCTT | 113 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCTATGGAGTAGTGGCCACAAACCACCAG | |
| | AGTGCCCAAGCACAGGCGCAGGTTGGCGCGTTGCAAACCCAAGGA | |
| | GTACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-41 | ATGGCTGCCGATGGTTATCTTCCAGATTGGCTCGAGGACAACCTT | 114 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCACTGGAGTCGTGGCCACAAACCACCAG | |
| | AGTGCCCAATCCCAGGCGGCGGTAGGCGCCCTACAAAGTCAAGGA | |
| | GTTCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-42 | ATGGCTGCCGATGGTTATCTTCCAGATTGGCTCGAGGACAACCTT | 115 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCTATGGAACTGTGGCCACAAACAATCAG | |
| | AGTGCCCAAGCACAGGCGCAGGTAGGCGCGTTACAATCTCAAGGA | |
| | ATACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-43 | ATGGCTGCCGATGGTTATCTTCCAGATTGGCTCGAGGACAACCTT | 116 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCTATGGAGTGGTGGCCACAAACCACCAG | |
| | AGTGCCAATGCACAGGCGCAGGTCGGCGCATTGCAAACTCAAGGA | |
| | GTCCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-44 | ATGGCTGCCGATGGTTATCTTCCAGATTGGCTCGAGGACAACCTT | 117 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCTATGGAGTCGTGGCCACAAACCACCAG | |
| | AGTGCCAACGCACAGGCGCAGGCGGGCGCCCTGCAATCCCAAGGA | |
| | GTGCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-45 | ATGGCTGCCGATGGTTATCTTCCAGATTGGCTCGAGGACAACCTT | 118 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCTATGGAACTGTGGCCACAAACCACCAG | |
| | AGTGCCAACACCCAGGCGAATGTGGGCGCCTTGCAATCCCAAGGA | |
| | GTCCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-46 | ATGGCTGCCGATGGTTATCTTCCAGATTGGCTCGAGGACAACCTT | 119 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGATTAGACAACGTGGATGCG | |
| | GACAAAGTCATGATAACCAACGAAGAAGAAATTAAAACTACTAAC | |
| | CCGGTAGCAACGGAGTCCTATGGAACCGTGGCCACAAACCACCAG | |
| | AGTGCCCAAGCACAGGCGCAGGTGGGCGCGCTGCAATCGCAAGGA | |
| | GTTCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-47 | ATGGCTGCCGATGGTTATCTTCCAGATTGGCTCGAGGACAACCTT | 120 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAACGTCATGATAACCAACGAAGAAGAAATTAAAACTACTAAC | |
| | CCGGTAGCAACGGAGTCCTATGGAGTAGTGGCCACAAACCACCAG | |
| | AGTGCCAATGCACAGGCGCAGGCCGGCGCGCTCCAATCCCAAGGA | |
| | GTGCTTCCGGGTATGGTTTGGCAGAACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-48 | ATGGCTGCCGATGGTTATCTTCCAGATTGGCTCGAGGACAACCTT | 121 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCTATGGAACAGTGGCCACAAACCACCAG | |
| | CAGTCACAAGCACAGGCGCAGGTGGGCGCTCTTCAATCTCAAGGA | |
| | GTGCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-49 | ATGGCTGCCGATGGTTATCTTCCAGATTGGCTCGAGGACAACCTT | 122 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCTATGGAACCGTGGCCACAAACCACCAG | |
| | AGTGCCAATACGCAGGCGGCGGTGGGCGCTCTCCAATCCCAAGGA | |
| | ATACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-50 | ATGGCTGCCGATGGTTATCTTCCAGATTGGCTCGAGGACAACCTT | 123 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCTATGGAGTTGTGGCCACAAACCACCAG | |
| | AGTAGCCAAGCACAGGCGCAGAGCGGCGCACTACAATCACAAGGA | |
| | GTTCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-51 | ATGGCTGCCGATGGTTATCTTCCAGATTGGCTCGAGGACAACCTT | 124 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCTATGGAACGGTGGCCACAAACCACCAG | |
| | AGTGCCAATACACAGGCGCAGGTTGGCGCGCTTCAATCTCAAGGA | |
| | GTACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-52 | ATGGCTGCCGATGGTTATCTTCCAGATTGGCTCGAGGACAACCTT | 125 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTAATAGAGACAACGTGGATGCG | |
| | GACAAAGTCATGATAACCAACGAAGAAGAAATTAAAACTACTAAC | |
| | CCGGTAGCAACGGAGTCCTATGGAACAGTGGCCACAAACCACCAG | |
| | AGTAGTCAAACACAGGCGCAGGTCGGCGCGCTACAAAGTCAAGGA | |
| | ATACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-53 | ATGGCTGCCGATGGTTATCTTCCAGATTGGCTCGAGGACAACCTT | 126 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACCTTGTCCAGATAACCAACGAAGAAGAAATTAAAACTACTAAC | |
| | CCGGTAGCAACGGAGTCCTATGGAGTGGTGGCCACAAACCACCAG | |
| | AGTGCCCAAGCACAGGCGCAGGCGGGCGCACTTCAAAGCCAAGGA | |
| | ATACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-54 | ATGGCTGCCGATGGTTATCTTCCAGATTGGCTCGAGGACAACCTT | 127 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTGGTATGGAACTGTGAATACAAACCACCAG | |
| | AGTGCCCAAGCACAGGCGCAGGTCGGCGCGCTGCAATCACAAGGA | |
| | GTACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-55 | ATGGCTGCCGATGGTTATCTTCCAGATTGGCTCGAGGACAACCTT | 128 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCTATGGAGTGGTGGCCACAAACAATCAG | |
| | AGTGCCCAAGCACAGGCGCAGGTAGGCGCTCTACAAAGTCAAGGA | |
| | GTCCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-60 | ATGGCTGCCGATGGTTATCTTCCAGATTGGCTCGAGGACAACCTT | 129 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCGCCATAACCAACGAAGAAGAAATTAAAACTACTAAC | |
| | CCGGTAGCAACGGAGTCCTATGGAACTGTGGCCACAAACCACCAG | |
| | CAGACCCAAGCACAGGCGCAGGTGGGCGCTCTGCAATCACAAGGA | |
| | ATACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-61 | ATGGCTGCCGATGGTTATCTTCCAGATTGGCTCGAGGACAACCTT | 130 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCACTATAACCAACGAAGAAGAAATTAAAACTACTAAC | |
| | CCGGTAGCAACGGAGTCCTATGGAGTAGTGGCCACAAACCACCAG | |
| | AGTTCGAACGCACAGGCGCAGAGCGGCGCCCTCCAATCACAAGGA | |
| | ATACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-57 | ATGGCTGCCGATGGTTATCTTCCAGATTGGCTCGAGGACAACCTT | 131 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATTATAACCAACGAAGAAGAAATTAAAACTACTAAC | |
| | CCGGTAGCAACGGAGTCCTATGGAAGCGTGGCCACAAACCACCAG | |
| | AGTGCCCAAGCACAGGCGGCCACCGGCTGGCTACAATCCCAAGGA | |
| | GCTCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-56 | ATGGCTGCCGATGGTTATCTTCCAGATTGGCTCGAGGACAACCTT | 132 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCTATGGATCAGTGGCCACAAACCTTCAG | |
| | AGTGCCCAAGCACAGGCGCAGGTTGGCTACCTGCAAAGCCAAGGA | |
| | GTCCTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-58 | ATGGCTGCCGATGGTTATCTTCCAGATTGGCTCGAGGACAACCTT | 133 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCTATGGATCGGTGGCCACAAACCACCAG | |
| | AGTGCCCAAGCACAGCCTCAGACCGGCTGGTTACAATCCCAAGGA | |
| | GCACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-59 | ATGGCTGCCGATGGTTATCTTCCAGATTGGCTCGAGGACAACCTT | 134 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGTGGTCCTATGGACAAGTGGCCACAAACAACCAG | |
| | AGTGCCCAAGCACAGGCGCAGGTAGGCGCACTACAATCTCAAGGA | |
| | GTACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-62 | ATGGCTGCCGATGGTTATCTTCCAGATTGGCTCGAGGACAACCTT | 135 |
| | AGTGAAGGTATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGTCTTCCTATGGACAAGTGGCCACAAACATGCAG | |
| | AGTGCCCAAGCACAGGCGCAGGTTGGCGCCTTACAATCTCAAGGA | |
| | ATACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGTAA | |
| VAR-B1 | MAADGYLPDWLEDNLSEGIREWWALKPGAPQPKANQQHQDNARGL | 136 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAIVGALQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B2 | MAADGYLPDWLEDNLSEGIREWWALKPGAPQPKANQQHQDNARGL | 137 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQVGALQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B3 | MAADGYLPDWLEDNLSEGIREWWALKPGAPQPKANQQHQDNARGL | 138 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQVGWLQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B4 | MAADGYLPDWLEDNLSEGIREWWALKPGAPQPKANQQHQDNARGL | 139 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQTGALQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B5 | MAADGYLPDWLEDNLSEGIREWWALKPGAPQPKANQQHQDNARGL | 140 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAIVGWLQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B6 | MAADGYLPDWLEDNLSEGIREWWALKPGAPQPKANQQHQDNARGL | 141 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAITGALQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B7 | MAADGYLPDWLEDNLSEGIREWWALKPGAPQPKANQQHQDNARGL | 142 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAIVGALQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B8 | MAADGYLPDWLEDNLSEGIREWWALKPGAPQPKANQQHQDNARGL | 143 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQVGWLQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B9 | MAADGYLPDWLEDNLSEGIREWWALKPGAPQPKANQQHQDNARGL | 144 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQVGALQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B10 | MAADGYLPDWLEDNLSEGIREWWALKPGAPQPKANQQHQDNARGL | 145 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAIVGWLQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B11 | MAADGYLPDWLEDNLSEGIREWWALKPGAPQPKANQQHQDNARGL | 146 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAIVGALQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B12 | MAADGYLPDWLEDNLSEGIREWWALKPGAPQPKANQQHQDNARGL | 147 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQVGALQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B13 | MAADGYLPDWLEDNLSEGIREWWALKPGAPQPKANQQHQDNARGL | 148 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQVGWLQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B14 | MAADGYLPDWLEDNLSEGIREWWALKPGAPQPKANQQHQDNARGL | 149 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQTGWLQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B15 | MAADGYLPDWLEDNLSEGIREWWALKPGAPQPKANQQHQDNARGL | 150 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAITGWLQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B16 | MAADGYLPDWLEDNLSEGIREWWALKPGAPQPKANQQHQDNARGL | 151 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAIVGWLQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B17 | MAADGYLPDWLEDNLSEGIREWWALKPGAPQPKANQQHQDNARGL | 152 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAIVGAVQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B18 | MAADGYLPDWLEDNLSEGIREWWALKPGAPQPKANQQHQDNARGL | 153 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAIVGWLQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B19 | MAADGYLPDWLEDNLSEGIREWWALKPGAPQPKANQQHQDNARGL | 154 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAIVGALQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B20 | MAADGYLPDWLEDNLSEGIREWWALKPGAPQPKANQQHQDNARGL | 155 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQTGAVQNQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B21 | MAADGYLPDWLEDNLSEGIREWWALKPGAPQPKANQQHQDNARGL | 156 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAITGWLQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B22 | MAADGYLPDWLEDNLSEGIREWWALKPGAPQPKANQQHQDNARGL | 157 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQTGALQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B23 | MAADGYLPDWLEDNLSEGIREWWALKPGAPQPKANQQHQDNARGL | 158 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQTGWLQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B24 | MAADGYLPDWLEDNLSEGIREWWALKPGAPQPKANQQHQDNARGL | 159 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAIVGAVQNQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B25 | MAADGYLPDWLEDNLSEGIREWWALKPGAPQPKANQQHQDNARGL | 160 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQTGALQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B26 | MAADGYLPDWLEDNLSEGIREWWALKPGAPQPKANQQHQDNARGL | 161 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAITGALQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B27 | MAADGYLPDWLEDNLSEGIREWWALKPGAPQPKANQQHQDNARGL | 162 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQVGALQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B28 | MAADGYLPDWLEDNLSEGIREWWALKPGAPQPKANQQHQDNARGL | 163 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAITGWVQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B29 | MAADGYLPDWLEDNLSEGIREWWALKPGAPQPKANQQHQDNARGL | 164 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQTGWLQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B30 | MAADGYLPDWLEDNLSEGIREWWALKPGAPQPKANQQHQDNARGL | 165 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQTGWLQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B31 | MAADGYLPDWLEDNLSEGIREWWALKPGAPQPKANQQHQDNARGL | 166 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQVGWVQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B32 | MAADGYLPDWLEDNLSEGIREWWALKPGAPQPKANQQHQDNARGL | 167 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAITGAVQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B33 | MAADGYLPDWLEDNLSEGIREWWALKPGAPQPKANQQHQDNARGL | 168 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQVGWLQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B34 | MAADGYLPDWLEDNLSEGIREWWALKPGAPQPKANQQHQDNARGL | 169 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAITGAVQNQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B35 | MAADGYLPDWLEDNLSEGIREWWALKPGAPQPKANQQHQDNARGL | 170 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQTGWLQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B36 | MAADGYLPDWLEDNLSEGIREWWALKPGAPQPKANQQHQDNARGL | 171 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAIVGAVQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B37 | MAADGYLPDWLEDNLSEGIREWWALKPGAPQPKANQQHQDNARGL | 172 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAITGWLQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B38 | MAADGYLPDWLEDNLSEGIREWWALKPGAPQPKANQQHQDNARGL | 173 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAITGWLQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B39 | MAADGYLPDWLEDNLSEGIREWWALKPGAPQPKANQQHQDNARGL | 174 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAIVGALQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B40 | MAADGYLPDWLEDNLSEGIREWWALKPGAPQPKANQQHQDNARGL | 175 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQVGWVQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B41 | MAADGYLPDWLEDNLSEGIREWWALKPGAPQPKANQQHQDNARGL | 176 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQVGAVQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B42 | MAADGYLPDWLEDNLSEGIREWWALKPGAPQPKANQQHQDNARGL | 177 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQTGWVQNQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B43 | MAADGYLPDWLEDNLSEGIREWWALKPGAPQPKANQQHQDNARGL | 178 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAIVGAVQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B44 | MAADGYLPDWLEDNLSEGIREWWALKPGAPQPKANQQHQDNARGL | 179 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAIVGWVQNQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B45 | MAADGYLPDWLEDNLSEGIREWWALKPGAPQPKANQQHQDNARGL | 180 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQTGALQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B46 | MAADGYLPDWLEDNLSEGIREWWALKPGAPQPKANQQHQDNARGL | 181 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQVGAVQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B47 | MAADGYLPDWLEDNLSEGIREWWALKPGAPQPKANQQHQDNARGL | 182 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQTGALQNQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B48 | MAADGYLPDWLEDNLSEGIREWWALKPGAPQPKANQQHQDNARGL | 183 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQTGWVQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B49 | MAADGYLPDWLEDNLSEGIREWWALKPGAPQPKANQQHQDNARGL | 184 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQVGAVQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B50 | MAADGYLPDWLEDNLSEGIREWWALKPGAPQPKANQQHQDNARGL | 185 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQVGALQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B51 | MAADGYLPDWLEDNLSEGIREWWALKPGAPQPKANQQHQDNARGL | 186 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAITGALQNQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B52 | MAADGYLPDWLEDNLSEGIREWWALKPGAPQPKANQQHQDNARGL | 187 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQTGWLQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B53 | MAADGYLPDWLEDNLSEGIREWWALKPGAPQPKANQQHQDNARGL | 188 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQTGWLQNQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B54 | MAADGYLPDWLEDNLSEGIREWWALKPGAPQPKANQQHQDNARGL | 189 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQVGALQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B55 | MAADGYLPDWLEDNLSEGIREWWALKPGAPQPKANQQHQDNARGL | 190 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQVGALQNQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B56 | MAADGYLPDWLEDNLSEGIREWWALKPGAPQPKANQQHQDNARGL | 191 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAITGWVQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B57 | MAADGYLPDWLEDNLSEGIREWWALKPGAPQPKANQQHQDNARGL | 192 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAIVGWVQNQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B58 | MAADGYLPDWLEDNLSEGIREWWALKPGAPQPKANQQHQDNARGL | 193 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQVGALQNQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B59 | MAADGYLPDWLEDNLSEGIREWWALKPGAPQPKANQQHQDNARGL | 194 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQTGAVQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B60 | MAADGYLPDWLEDNLSEGIREWWALKPGAPQPKANQQHQDNARGL | 195 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQTGALQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B61 | MAADGYLPDWLEDNLSEGIREWWALKPGAPQPKANQQHQDNARGL | 196 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQTGWVQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B62 | MAADGYLPDWLEDNLSEGIREWWALKPGAPQPKANQQHQDNARGL | 197 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAITGAVQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B63 | MAADGYLPDWLEDNLSEGIREWWALKPGAPQPKANQQHQDNARGL | 198 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQVGAVQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B64 | MAADGYLPDWLEDNLSEGIREWWALKPGAPQPKANQQHQDNARGL | 199 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQVGAVQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B65 | MAADGYLPDWLEDNLSEGIREWWALKPGAPQPKANQQHQDNARGL | 200 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAIVGWVQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B66 | MAADGYLPDWLEDNLSEGIREWWALKPGAPQPKANQQHQDNARGL | 201 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAITGWVQNQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B67 | MAADGYLPDWLEDNLSEGIREWWALKPGAPQPKANQQHQDNARGL | 202 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQTGWVQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B68 | MAADGYLPDWLEDNLSEGIREWWALKPGAPQPKANQQHQDNARGL | 203 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAIVGWVQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B69 | MAADGYLPDWLEDNLSEGIREWWALKPGAPQPKANQQHQDNARGL | 204 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQTGAVQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B70 | MAADGYLPDWLEDNLSEGIREWWALKPGAPQPKANQQHQDNARGL | 205 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAIVGWLQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B71 | MAADGYLPDWLEDNLSEGIREWWALKPGAPQPKANQQHQDNARGL | 206 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAIVGWVQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B72 | MAADGYLPDWLEDNLSEGIREWWALKPGAPQPKANQQHQDNARGL | 207 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQVGWLQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B73 | MAADGYLPDWLEDNLSEGIREWWALKPGAPQPKANQQHQDNARGL | 208 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQVGWLQNQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B74 | MAADGYLPDWLEDNLSEGIREWWALKPGAPQPKANQQHQDNARGL | 209 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAITGWLQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B75 | MAADGYLPDWLEDNLSEGIREWWALKPGAPQPKANQQHQDNARGL | 210 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAITGWLQNQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B76 | MAADGYLPDWLEDNLSEGIREWWALKPGAPQPKANQQHQDNARGL | 211 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQTGWLQNQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B77 | MAADGYLPDWLEDNLSEGIREWWALKPGAPQPKANQQHQDNARGL | 212 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAITGALQNQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B78 | MAADGYLPDWLEDNLSEGIREWWALKPGAPQPKANQQHQDNARGL | 213 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAIVGALQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B79 | MAADGYLPDWLEDNLSEGIREWWALKPGAPQPKANQQHQDNARGL | 214 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQVGWVQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B80 | MAADGYLPDWLEDNLSEGIREWWALKPGAPQPKANQQHQDNARGL | 215 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAITGAVQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B81 | MAADGYLPDWLEDNLSEGIREWWALKPGAPQPKANQQHQDNARGL | 216 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAIVGWVQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B82 | MAADGYLPDWLEDNLSEGIREWWALKPGAPQPKANQQHQDNARGL | 217 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQTGWVQNQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B83 | MAADGYLPDWLEDNLSEGIREWWALKPGAPQPKANQQHQDNARGL | 218 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAIVGWVQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B84 | MAADGYLPDWLEDNLSEGIREWWALKPGAPQPKANQQHQDNARGL | 219 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAITGAVQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B85 | MAADGYLPDWLEDNLSEGIREWWALKPGAPQPKANQQHQDNARGL | 220 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQTGAVQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B86 | MAADGYLPDWLEDNLSEGIREWWALKPGAPQPKANQQHQDNARGL | 221 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAITGALQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B87 | MAADGYLPDWLEDNLSEGIREWWALKPGAPQPKANQQHQDNARGL | 222 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQTGALQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B88 | MAADGYLPDWLEDNLSEGIREWWALKPGAPQPKANQQHQDNARGL | 223 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAIVGALQNQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B89 | MAADGYLPDWLEDNLSEGIREWWALKPGAPQPKANQQHQDNARGL | 224 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAITGWVQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B99 | MAADGYLPDWLEDNLSEGIREWWALKPGAPQPKANQQHQDNARGL | 225 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAITGALQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B100 | MAADGYLPDWLEDNLSEGIREWWALKPGAPQPKANQQHQDNARGL | 226 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAIVGAVQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B101 | MAADGYLPDWLEDNLSEGIREWWALKPGAPQPKANQQHQDNARGL | 227 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAITGALQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B102 | MAADGYLPDWLEDNLSEGIREWWALKPGAPQPKANQQHQDNARGL | 228 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQTGALQNQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B103 | MAADGYLPDWLEDNLSEGIREWWALKPGAPQPKANQQHQDNARGL | 229 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAITGAVQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B104 | MAADGYLPDWLEDNLSEGIREWWALKPGAPQPKANQQHQDNARGL | 230 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQVGWVQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B105 | MAADGYLPDWLEDNLSEGIREWWALKPGAPQPKANQQHQDNARGL | 231 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQVGWVQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B106 | MAADGYLPDWLEDNLSEGIREWWALKPGAPQPKANQQHQDNARGL | 232 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQTGAVQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B107 | MAADGYLPDWLEDNLSEGIREWWALKPGAPQPKANQQHQDNARGL | 233 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAITGWVQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B108 | MAADGYLPDWLEDNLSEGIREWWALKPGAPQPKANQQHQDNARGL | 234 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQTGWVQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B109 | MAADGYLPDWLEDNLSEGIREWWALKPGAPQPKANQQHQDNARGL | 235 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQTGAVQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B110 | MAADGYLPDWLEDNLSEGIREWWALKPGAPQPKANQQHQDNARGL | 236 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQTGAVQNQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B111 | MAADGYLPDWLEDNLSEGIREWWALKPGAPQPKANQQHQDNARGL | 237 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQVGAVQNQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B112 | MAADGYLPDWLEDNLSEGIREWWALKPGAPQPKANQQHQDNARGL | 238 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQVGWVQNQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B113 | MAADGYLPDWLEDNLSEGIREWWALKPGAPQPKANQQHQDNARGL | 239 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAITGAVQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B114 | MAADGYLPDWLEDNLSEGIREWWALKPGAPQPKANQQHQDNARGL | 240 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAITGWVQNQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B115 | MAADGYLPDWLEDNLSEGIREWWALKPGAPQPKANQQHQDNARGL | 241 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQTGAVQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B116 | MAADGYLPDWLEDNLSEGIREWWALKPGAPQPKANQQHQDNARGL | 242 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAIVGWVQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B117 | MAADGYLPDWLEDNLSEGIREWWALKPGAPQPKANQQHQDNARGL | 243 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAITGAVQNQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B118 | MAADGYLPDWLEDNLSEGIREWWALKPGAPQPKANQQHQDNARGL | 244 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAITGWVQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B119 | MAADGYLPDWLEDNLSEGIREWWALKPGAPQPKANQQHQDNARGL | 245 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAIVGAVQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B120 | MAADGYLPDWLEDNLSEGIREWWALKPGAPQPKANQQHQDNARGL | 246 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQVGWVQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B121 | MAADGYLPDWLEDNLSEGIREWWALKPGAPQPKANQQHQDNARGL | 247 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGVVATNHQ | |
| | SAQAQAQTGWVQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B122 | MAADGYLPDWLEDNLSEGIREWWALKPGAPQPKANQQHQDNARGL | 248 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAITGWVQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B123 | MAADGYLPDWLEDNLSEGIREWWALKPGAPQPKANQQHQDNARGL | 249 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQVGAVQSQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B124 | MAADGYLPDWLEDNLSEGIREWWALKPGAPQPKANQQHQDNARGL | 250 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQVGWLQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B125 | MAADGYLPDWLEDNLSEGIREWWALKPGAPQPKANQQHQDNARGL | 251 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAITGALQSQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| VAR-B126 | MAADGYLPDWLEDNLSEGIREWWALKPGAPQPKANQQHQDNARGL | 252 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAIVGWLQNQGALPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| CBh promoter | CCAACCTGAAAAAAAGTGATTTCAGGCAGGTGCTCCAGGTAATTA | 253 |
| | AACATTAATACCCCACCAACCAACCATCCCTTAAACCCTTACCTC | |
| | TTGCTCAGCTAATTACAGCCCGGAGGAGAAGGGCCGTCCCGCCCG | |
| | CTCACCTGTGGGAGTAACGCGGTCAGTCAGAGCCGGGGGGGGCGG | |
| | CGCGAGGCGGCGGCGGAGCGGGGCACGGGGCGAAGGCAGCGCGCA | |
| | GCGACTCCCGCCCGCCGCGCGCTTCGCTTTTTATAGGGCCGCCGC | |
| | CGCCGCCGCCTCGCCATAAAAGGAAACTTTCGGAGCGCGCCGCTC | |
| | TGATTGGCTGCCGCCGCACCTCTCCGCCTCGCCCCGCCCCGCCCC | |
| | TCGCCCCGCCCCGCCCCGCCTGGCGCGCGCCCCCCCCCCCCCCCC | |
| | GCCCCCATCGCTGCACAAAATAATTAAAAAATAAATAAATACAAA | |
| | ATTGGGGGTGGGGAGGGGGGGGAGATGGGGAGAGTGAAGCAGAAC | |
| | GTGGGGCTCACCTCGACCATGGTAATAGCGATGACTAATACGTAG | |
| | ATGTACTGCCAAGTAGGAAAGTCCCATAAGGTCATGTACTGGGCA | |
| | CAATGCCAGGCGGGCCATTTACCGTCATTGACGTCAATAGGGGGC | |
| | GTACTTGGCATATGATACACTTGATGTACTGCCAAGTGGGCAGTT | |
| | TACCGTAAATACTCCACCCATTGACGTCAATGGAAAGTCCCTATT | |
| | GGCGTTACTATTGACGTCAATGGGGGGGGGTCGTTGGGCGGTCAG | |
| | CCAGGGGGGCCATTTACCGTAAGTTATGTAACG | |
| hSYN promoter | AGTGCAAGTGGGTTTTAGGACCAGGATGAGGCGGGGGGGGGTGCC | 254 |
| | TACCTGACGACCGACCCCGACCCACTGGACAAGCACCCAACCCCC | |
| | ATTCCCCAAATTGCGCATCCCCTATCAGAGAGGGGGAGGGGAAAC | |
| | AGGATGCGGCGAGGCGCGTGCGCACTGCCAGCTTCAGCACCGCGG | |
| | ACAGTGCCTTCGCCCCCGCCTGGCGGCGCGCGCCACCGCCGCCTC | |
| | AGCACTGAAGGCGCGCTGACGTCACTCGCCGGTCCCCCGCAAACT | |
| | CCCCTTCCCGGCCACCTTGGTCGCGTCCGCGCCGCCGCCGGCCCA | |
| | GCCGGACCGCACCACGCGAGGCGCGAGATAGGGGGGCACGGGCGC | |
| | GACCATCTGCGCTGCGGCGCCGGCGACTCAGCGCTGCCTCAGTCT | |
| | GCGGTGGGCAGCGGAGGAGTCGTGTCGTGCCTGAGAGCGCAG | |
| Targeting | PLNGAVHLY | 255 |
| peptide | | |
| Targeting | IVMNSLK | 256 |
| peptide | | |
| Targeting | RDSPKGW | 257 |
| peptide | | |
| Targeting | YSTDVRM | 258 |
| peptide | | |
| Targeting | RESPRGL | 259 |
| peptide | | |
| Targeting | GNNTRSV | 260 |
| peptide | | |
| Targeting | GNNTRDT | 261 |
| peptide | | |
| Targeting | TNSTRPV | 262 |
| peptide | | |
| Targeting | ASSLNIA | 263 |
| peptide | | |
| Targeting | SLEEEWAQVECEVYGRGCPSGSLDESFYDWFERQL | 264 |
| peptide | | |
| Targeting | TPFYPVGYTVKQPGTCGDGVLQPGEECDDGNPDVSDGCIDCHRAY | 265 |
| peptide | CGDGYRHQGVEDCDGSDFGYLTCETYLPGSYGDLRCTQYCSIDST | |
| | PCRYFT | |
| Targeting | RGDY | 266 |
| peptide | | |
| Targeting | RGDF | 26 |
| peptide | | |
| Targeting | RGDX₁X₂X₃X₄, | N/A |
| peptide | with X₁ to X₄ each being any amino acid. | |
| | In various aspects, X₁, X₂, and X₃ are | |
| | each independently selected from L, | |
| | G, V, and A and/or X₄ is S, V, A, G, or L. | |
| | In some embodiments, at least | |
| | one of X₂ and X₃ is G. | |
| Targeting | RGDLGLS | 269 |
| peptide | | |
| Targeting | RGDLSTP | 270 |
| peptide | | |
| Targeting | SNSRGDYNSL | 271 |
| peptide | | |
| Targeting | ENRRGDFNNT | 272 |
| peptide | | |
| Targeting | SRGDYNSL | 273 |
| peptide | | |
| Targeting | RGDYNSL | 274 |
| peptide | | |
| Targeting | RGDLST | 275 |
| peptide | | |
| Targeting | RGDYVGL | 276 |
| peptide | | |
| Targeting | RGDAVGV | 277 |
| peptide | | |
| Linker | GGGS | 278 |

### 9. CITATION OF REFERENCES

All publications, patent applications, patents, and other publications and references (e.g., sequence database reference numbers) cited herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Aug. 21, 2020. When one gene or protein references a plurality of sequence accession numbers, all of the sequence variants are encompassed.

What is claimed is:

1. A capsid polypeptide comprising a valine at a position corresponding to 1601 of the VP1 capsid polypeptide of SEQ ID NO:1, and optionally one, two or three or all four of:
(a) an alanine, arginine, serine, threonine, valine at a position corresponding to T593 of the VP1 capsid polypeptide of SEQ ID NO:1;
(b) an alanine, tryptophan, or tyrosine at a position corresponding to W595 of the VP1 capsid polypeptide of SEQ ID NO: 1
(c) a leucine at a position corresponding to V596 of the VP1 capsid polypeptide of SEQ ID NO:1; and
(d) a serine or threonine at a position corresponding to N598 of the VP1 capsid polypeptide of SEQ ID NO:1.

2. A capsid polypeptide comprising:
(a) alanine or serine at a position corresponding to T593 of the VP1 capsid polypeptide of SEQ ID NO:1;
(b) a leucine at a position corresponding to V596 of the VP1 capsid polypeptide of SEQ ID NO:1; and
(c) a serine or threonine at a position corresponding to N598 of the VP1 capsid polypeptide of SEQ ID NO:1.

3. The capsid polypeptide of claim 2, which comprises a serine at a position corresponding to N598 of the VP1 capsid polypeptide of SEQ ID NO:1.

4. The capsid polypeptide of claim 2, which comprises a threonine at a position corresponding to N598 of the VP1 capsid polypeptide of SEQ ID NO:1.

5. A capsid polypeptide comprising:
(a) an alanine, tryptophan, or tyrosine at a position corresponding to W595 of the VP1 capsid polypeptide of SEQ ID NO. 1
(b) a leucine at a position corresponding to V596 of the VP1 capsid polypeptide of SEQ ID NO:1; and
(c) a threonine at a position corresponding to N598 of the VP1 capsid polypeptide of SEQ ID NO:1.

6. The capsid polypeptide of claim 5, which comprises an alanine at a position corresponding to W595 of the VP1 capsid polypeptide of SEQ ID NO:1.

7. A capsid polypeptide comprising:
(a) an alanine or serine at a position corresponding to T593 of the VP1 capsid polypeptide of SEQ ID NO:1;
(b) an alanine, tryptophan, or tyrosine at a position corresponding to W595 of the VP1 capsid polypeptide of SEQ ID NO: 1
(c) a leucine at a position corresponding to V596 of the VP1 capsid polypeptide of SEQ ID NO:1, and
(d) a serine or threonine at a position corresponding to N598 of the VP1 capsid polypeptide of SEQ ID NO:1.

8. A capsid polypeptide comprising:
(a) an alanine, arginine, serine, threonine, valine at a position corresponding to T593 of the VP1 capsid polypeptide of SEQ ID NO:1;
(b) an alanine, tryptophan, or tyrosine at a position corresponding to W595 of the VP1 capsid polypeptide of SEQ ID NO: 1
(c) a leucine at a position corresponding to V596 of the VP1 capsid polypeptide of SEQ ID NO:1; and
(d) a threonine at a position corresponding to N598 of the VP1 capsid polypeptide of SEQ ID NO:1.

9. The capsid polypeptide of any one of claims 1 to 8, which comprises a glutamic acid at a position corresponding to D551 of the VP1 capsid polypeptide of SEQ ID NO:1.

10. The capsid polypeptide of any one of claims 1 to 9, which comprises a threonine at a position corresponding to Q579 of the VP1 capsid polypeptide of SEQ ID NO:1.

11. The capsid polypeptide of any one of claims 1 to 9, which comprises a valine at a position corresponding to Q579 of the VP1 capsid polypeptide of SEQ ID NO:1.

12. The capsid polypeptide of any one of claims 1 to 11, which comprises an alanine at a position corresponding to Q592 of the VP1 capsid polypeptide of SEQ ID NO:1.

13. The capsid polypeptide of any one of claims 1 to 11, which comprises an asparagine at a position corresponding to Q592 of the VP1 capsid polypeptide of SEQ ID NO:1.

14. The capsid polypeptide of any one of claims 1 to 11, which comprises a glutamine at a position corresponding to Q592 of the VP1 capsid polypeptide of SEQ ID NO:1.

15. A capsid polypeptide comprising 6, 7 or all 8 mutations of the mutation set of VAR-2 as compared to the VP1 capsid polypeptide of SEQ ID NO:1, as set forth in Table 24, preferably wherein the mutations comprise the V596 and N598 mutations and optionally a mutation at Q579 and/or T593 as compared to the VP1 capsid polypeptide of SEQ ID NO:1.

16. A capsid polypeptide comprising 5, 6 or all 7 mutations of the mutation set of VAR-3 as compared to the VP1 capsid polypeptide of SEQ ID NO:1, as set forth in Table 24, preferably wherein the mutations comprise the V596 and N598 mutations and optionally a mutation at Q579 and/or T593 as compared to the VP1 capsid polypeptide of SEQ ID NO:1.

17. A capsid polypeptide comprising 4, 5 or all 6 mutations of the mutation set of VAR-11 as compared to the VP1 capsid polypeptide of SEQ ID NO:1, as set forth in Table 24, preferably wherein the mutations comprise the V596 and N598 mutations and optionally a mutation at Q579 and/or T593 as compared to the VP1 capsid polypeptide of SEQ ID NO:1.

18. A capsid polypeptide comprising 4, 5 or all 6 mutations of the mutation set of VAR-40 as compared to the VP1 capsid polypeptide of SEQ ID NO:1, as set forth in Table 24, preferably wherein the mutations comprise the V596 and N598 mutations and optionally a mutation at Q579 and/or T593 as compared to the VP1 capsid polypeptide of SEQ ID NO:1.

19. A capsid polypeptide comprising 4, 5 or all 6 mutations of the mutation set of VAR-42 as compared to the VP1 capsid polypeptide of SEQ ID NO:1, as set forth in Table 24, preferably wherein the mutations comprise the V596 and N598 mutations and optionally a mutation at Q579 and/or T593 as compared to the VP1 capsid polypeptide of SEQ ID NO:1.

20. A capsid polypeptide comprising 6, 7 or all 8 mutations of the mutation set of VAR-54 as compared to the VP1 capsid polypeptide of SEQ ID NO:1, as set forth in Table 24, preferably wherein the mutations comprise the V596 and N598 mutations and optionally a mutation at Q579 and/or T593 as compared to the VP1 capsid polypeptide of SEQ ID NO:1.

21. The capsid polypeptide of any one of claims 1 to 20, whose sequence has an edit distance of (a) 12 or lower or (b) 10 or lower to a VP1 capsid polypeptide of SEQ ID NO:1 or the VP2 or VP3 portion thereof.

22. The capsid polypeptide of any one of claims 1 to 20, which comprises an amino acid sequence having at least 95% sequence identity to a VP1 capsid polypeptide of SEQ ID NO 1 or the VP2 or VP3 portion thereof.

23. The capsid polypeptide of any one of claims 1 to 22, which is a VP1 capsid polypeptide.

24. The capsid polypeptide of any one of claims 1 to 22, which is a VP2 capsid polypeptide.

25. The capsid polypeptide of any one of claims 1 to 22, which is a VP3 capsid polypeptide.

26. A nucleic acid comprising a nucleotide sequence encoding a capsid polypeptide of any one of claims 1 to 25.

27. A virus particle comprising a capsid polypeptide of any one of claims 1 to 25.

28. The virus particle of claim 27, comprising a nucleic acid comprising a payload (e.g., a heterologous transgene) and one or more regulatory elements.

29. The virus particle of claim 28, wherein the one or more regulatory elements comprise a promoter.

30. The virus particle of claim 29, wherein the promoter is a constitutive promoter.

31. The virus particle of claim 29, wherein the promoter is a CNS-specific promoter.

32. The virus particle of claim 29, wherein the promoter is a muscle-specific promoter.

33. A host cell engineered comprising a nucleic acid encoding a capsid polypeptide according to any one of claims 1 to 25.

34. The host cell of claim 33, which is a packaging cell line.

35. The host cell of claim 33 or claim 34, which comprises a nucleic acid encoding a rep protein.

36. The host cell of any one of claims 33 to 35, which comprises a nucleic acid comprising one or more helper sequences.

37. The host cell of any one of claims 33 to 36, which is configured to package a virus particle according to any one of claims 27 to 32.

38. A method of producing a virus particle comprising a capsid polypeptide, said method comprising introducing a nucleic acid molecule according to claim 26 into a cell and harvesting said virus particles therefrom.

39. A method of producing a virus particle comprising a capsid polypeptide, comprising culturing a cell engineered to express a capsid polypeptide of any one of claims 1 to 25 and harvesting virus particles therefrom.

40. The method of claim 38 or claim 39, wherein the cell is a host cell according to any one of claims 33 to 37.

41. A method of delivering a payload (e.g., a nucleic acid) to a cell, comprising (a) contacting the cell with a virus particle comprising the capsid polypeptide of any one of claims 1 to 25 and a payload; or
(b) contacting the cell with the virus particle of any one of claims 27 to 32.

42. The method of claim 42, wherein the cell is a CNS cell.

43. A method of delivering a payload (e.g., a nucleic acid) to a subject, comprising:
(a) administering to the subject a virus particle comprising the capsid polypeptide of any one of claims 1 to 25 and the payload; or
(b) (b) administering to the subject the virus particle of any one of claims 27 to 32.

44. The method of claim 43, wherein the virus particle delivers the payload to the CNS.

45. A method of treating a disease or condition in a subject, comprising administering to the subject in an amount effective to treat the disease or condition:
(a) a virus particle comprising the capsid polypeptide of any one of claims 1 to 25 and a heterologous nucleic acid sequence encoding a therapeutic product suitable for treating the disease or condition;
(b) a virus particle comprising a capsid polypeptide encoded by the nucleic acid molecule of claim 26 and a heterologous nucleic acid sequence encoding a therapeutic product suitable for treating the disease or condition, or
(c) the virus particle of any one of claims 27 to 32; or
(d) a composition, e.g., a pharmaceutical composition, comprising the virus particle of (a) and, optionally, a pharmaceutically acceptable carrier.

46. The method of claim 45, wherein the disease or condition is a disease or condition of the CNS.
